(19) United States
(12) Patent Application Publication
(10) Pub. No.: US 2008/0004271 A1
(43) Pub. Date: Jan. 3, 2008

(54) Inhibitors of TNFalpha, PDE4 and B-RAF, compositions thereof and methods of use therewith

(76) Inventors: Jeffrey M. McKenna, Horsham (GB); Patrick W. Papa, Carlsbad, CA (US); Steven T. Sakata, San Diego, CA (US); Paul E. Erdman, San Diego, CA (US); Garrick K. Packard, San Diego, CA (US)

Correspondence Address: JONES DAY, 222 EAST 41ST ST, NEW YORK, NY, 10017, US

(21) Appl. No.: 11/654,344

(22) Filed: Jan. 16, 2007

Related U.S. Application Data
(60) Provisional application No. 60/759,819, filed on Jan. 17, 2006.
(60) Provisional application No. 60/814,862, filed on Jun. 19, 2006.
(60) Provisional application No. 60/818,246, filed on Jun. 30, 2006.
(60) Provisional application No. 60/854,637, filed on Oct. 25, 2006.

(51) Int. Cl. A61K 31/519; A61K 31/5377; A61P 35/00; C07D 413/14; C07D 495/04; C12N 5/00

(52) U.S. Cl. 514/234.2; 435/375; 514/260.1; 544/117; 544/278

(57) ABSTRACT

Provided herein are compounds having TNFα and/or PDE4 and/or B-RAF inhibitory activity, and compositions thereof. In particular, provided herein are compounds of the formula I: 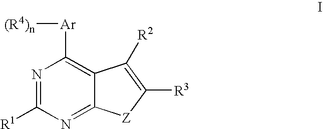
and pharmaceutically acceptable salts, solvates, hydrates, clathrates, stereoisomers, polymorphs and prodrugs thereof, wherein Ar, R¹, R², R³, R⁴, n and Z are as described herein. Further provided herein are methods for treating or preventing various diseases and disorders by administering to a patient one or more TNFα and/or PDE4 and/or B-RAF inhibitors. In particular, provided herein are methods for preventing or treating cancer, inflammatory disorders, cognition and memory disorders and autoimmune disorders, or one or more symptoms thereof by administering to a patient one or more TNFα and/or PDE4 and/or B-RAF inhibitors.

This application claims the benefit of U.S. provisional application No. 60/759,819, filed Jan. 17, 2006, U.S. provisional application No. 60/814,862, filed Jun. 19, 2006, U.S. provisional application No. 60/818,246, filed Jun. 30, 2006, and U.S. provisional application No. 60/854,637, filed Oct. 25, 2006, the disclosures of which are incorporated by reference herein in their entireties.

### 1 FIELD

Provided herein are compounds that have activity as TNFα inhibitors, PDE4 and/or B-RAF inhibitors, and compositions thereof. Also provided herein are methods for treating, preventing and/or managing various diseases and disorders by administering to a patient in need thereof one or more compounds described herein. In particular, provided herein are methods for treating, preventing or managing cancer, inflammatory disorders, cognition and memory disorders and autoimmune disorders by administering one or more compounds provided herein.

### 2 BACKGROUND

2.1 TNFα

Tumor necrosis factor alpha, (TNFα) is a cytokine which is released primarily by mono-nuclear phagocytes in response to immunostimulators. When administered to mammals, TNFα can cause inflammation, fever, cardiovascular effects, hemorrhage, coagulation, and acute phase responses similar to those seen during acute infections and shock states.

The nuclear factor κB (NFκB) is a pleiotropic transcriptional activator {Lenardo, et al., Cell 58, 227-29 (1989)} which has been implicated in a variety of disease and inflammatory states. NFκB is thought to regulate cytokine levels including, but not limited to, TNFα and to be an activator of HIV transcription {Dbaibo et al., J. Biol. Chem. 17762-66 (1993); Duh et al., Proc. Natl. Acad. Sci. 86, 5974-78 (1989); Bachelerie et al., Nature 350, 709-12 (1991); Boswas et al., J. Acquired Immune Deficiency Syndrome 6, 778-786 (1993); Suzuki et al., Biochem. And Biophys. Res. Comm. 193, 277-83 (1993); Suzuki et al., Biochem. And Biophys. Res. Comm. 189, 1709-15 (1992); Suzuki et al., Biochem. Mol. Bio. Int. 31(4), 693-700 (1993); Shakhov et al. 171, 35-47 (1990); and Staal et al., Proc. Natl. Acad. Sci. USA 87, 9943-47 (1990)}. Thus, inhibition of NFκB binding can regulate transcription of cytokine gene(s) and through this modulation and other mechanisms be useful in the treatment or prevention of a multitude of disease states.

Excessive or unregulated TNFα production has been implicated in a number of disease conditions. These include endotoxemia and/or toxic shock syndrome {Tracey et al., Nature 330, 662-664 (1987) and Hinshaw et al., Circ. Shock 30, 279-292 (1990)}; cachexia {Dezube et al., Lancet, 335 (8690), 662 (1990)}; and Adult Respiratory Distress Syndrome (ARDS) where TNFα concentrations in excess of 12,000 pg/milliliters have been detected in pulmonary aspirates from ARDS patients {Millar et al., Lancet 2 (8665), 712-714 (1989)}. Systemic infusion of recombinant TNFα. also resulted in changes typically seen in ARDS {Ferrai-Baliviera et al., Arch. Surg. 124(12), 1400-1405 (1989)}.

TNFα also appears to be involved in bone resorption diseases, including arthritis, where it has been determined that when activated, leukocytes will produce a bone-resorbing activity, and data suggests that TNFα contributes to this activity {Bertolini et al., Nature 319, 516-518 (1986) and Johnson et al., Endocrinology 124(3), 1424-1427 (1989)}. It has been determined that TNFα stimulates bone resorption and inhibits bone formation in vitro and in vivo through stimulation of osteoblast formation and activation in combination with inhibition of osteoblast function. Although TNFα may be involved in many bone resorption diseases, including arthritis, the most compelling link with disease is the association between production of TNFα by tumor or host tissues and malignancy associated hypercalcemia {Calci. Tissue Int. (US) 46 (Suppl.), S3-10 (1990)}. In Graft versus Host disease, increased serum TNFα levels have been associated with major complications following acute allogenic bone marrow transplants {Holler et al., Blood, 75(4), 1011-1016 (1990)}.

TNFα also appears to play a role in the area of chronic pulmonary inflammatory diseases. The deposition of silica particles leads to silicosis, a disease of progressive respiratory failure caused by a fibrotic reaction. Antibodies to TNFα completely blocked the silica-induced lung fibrosis in mice {Pignet et al., Nature, 344:245-247 (1990)}. High levels of TNFα production (in the serum and in isolated macrophages) have been demonstrated in animal models of silica and asbestos induced fibrosis {Bissonnette et al., Inflammation 13(3), 329-339 (1989)}. Alveolar macrophages from pulmonary sarcoidosis patients have also been found to spontaneously release massive quantities of TNFα as compared with macrophages from normal donors {Baughman et al., J. Lab. Clin. Med. 115 (1), 36-42 (1990)}.

TNFα is also implicated in the inflammatory response which follows reperfusion, called reperfusion injury, and is a major cause of tissue damage after loss of blood flow {Vedder et al., PNAS 87, 2643-2646 (1990)}. TNFα also alters the properties of endothelial cells and has various pro-coagulant activities, such as producing an increase in tissue factor pro-coagulant activity and suppression of the anticoagulant protein C pathway as well as down-regulating the expression of thrombomodulin {Sherry et al., J. Cell Biol. 107, 1269-1277 (1988)}. TNFα has pro-inflammatory activities which together with its early production (during the initial stage of an inflammatory event) make it a likely mediator of tissue injury in several important disorders including but not limited to, myocardial infarction, stroke and circulatory shock. Of specific importance may be TNFα-induced expression of adhesion molecules, such as intercellular adhesion molecule (ICAM) or endothelial leukocyte adhesion molecule (ELAM) on endothelial cells {Munro et al., Am. J. Path. 135 (1), 121-132 (1989)}.

High levels of TNFα are associated with Crohn's disease {von Dullemen et al., Gastroenterology, 1995 109(1), 129-135} and clinical benefit has been achieved with TNFα antibody treatment, thus confirming the importance of TNFα in the disease. TNFα has also been implicated in other viral infections, such as the cytomegalia virus (CMV), influenza virus, adenovirus, and the herpes family of viruses for similar reasons as those noted.

Adenosine 3′,5′-cyclic monophosphate (cAMP) is an enzyme that plays a role in many diseases and conditions, such as, but not limited to asthma and inflammation {Lowe and Cheng, Drugs of the Future, 17(9), 799-807, 1992)}. The elevation of cAMP in inflammatory leukocytes reportedly inhibits their activation and the subsequent release of inflammatory mediators, including TNF-α and nuclear factor κB (NF-κB). Increased levels of cAMP also lead to the relaxation of airway smooth muscle.

2.2 PDE4

It is believed that a primary cellular mechanism for the inactivation of cAMP is the breakdown of cAMP by a family of isoenzymes referred to as cyclic nucleotide phosphodiesterases (PDE) {Beavo and Reitsnyder, Trends in Pharm., 11, 150-155 (1990)}. There are twelve known members of the family of PDEs. It is recognized that the inhibition of PDE type IV (PDE4) is particularly effective in both the inhibition of inflammatory mediated release and the relaxation of airway smooth muscle {Verghese, et al., Journal of Pharmacology and Experimental Therapeutics, 272(3), 1313-1320 (1995)}. Thus, compounds that specifically inhibit PDE4 may inhibit inflammation and aid the relaxation of airway smooth muscle with a minimum of unwanted side effects, such as cardiovascular or anti-platelet effects.

The PDE 4 family that is specific for cAMP is currently the largest, and is composed of at least 4 isozymes (a-d), and multiple splice variants {Houslay, M. D. et al. in Advances in Pharmacology 44, eds. J. August et al., p. 225 (1998)}. There may be over 20 PDE4 isoforms expressed in a cell specific pattern regulated by a number of different promoters. Disease states for which selective PDE4 inhibitors have been sought include: asthma, atopic dermatitis, depression, reperfusion injury, septic shock, toxic shock, endotoxic shock, adult respiratory distress syndrome, autoimmune diabetes, diabetes insipidus, multi-infarct dementia, AIDS, cancer, Crohn's disease, multiple sclerosis, cerebral ischemia, psoriasis, allograft rejection, restenosis, ulcerative colitis, cachexia, cerebral malaria, allergic rhino-conjunctivitis, osteoarthritis, rheumatoid arthritis, chronic obstructive pulmonary disease (COPD), chronic bronchitis, cosinophilic granuloma, and autoimmune encephalomyelitis {Id.}. PDE4 is present in the brain and major inflammatory cells and has been found in abnormally elevated levels in a number of diseases including atopic dermatitis or eczema, asthma, and hay fever among others {Grewe et al., J. of Allergy and Clinical Immunology, 70: 452-457 (1982)}. In individuals suffering from atopic diseases elevated PDE-4 activity is found in their peripheral blood mononuclear leukocytes, T cells, mast cells, neutrophils and basophils. This increased PDE activity decreases cAMP levels and results in a breakdown of cAMP control in these cells. This results in increased immune responses in the blood and tissues of those that are affected.

The activation of cAMP was proposed as a promising strategy for inducing neurons to overcome inhibitory signals after SCI. {Damien D Pearse et al., Nature Medicine; 10(6):610-616 (2004)}. The authors showed that inhibition of cAMP hydrolysis by rolipram, a PDE4 inhibitor, prevented the decrease of cAMP levels after spinal cord contusion, and that when combined with Schwann cell grafts, it promoted significant supraspinal and proprioceptive axon sparing and myelination. Further, it was shown that combination of rolipram with db-cAMP, a cAMP analog, increased cAMP levels above those in uninjured control subjects, enhances axonal sparing, myelination and growth of serotonergic fibers, and improved locomotion. Id.

PDE4 inhibitors have also been shown to influence eosinophil responses, decrease basophil histamine release, decrease IgE, PGE2, IL10 synthesis, and decrease anti-CD3 stimulated Il-4 production. Similarly, PDE4 inhibitors have been shown to block neutrophil functions. Neutrophils play a major role in asthma, COPD and other allergic disorders. PDE4 inhibitors have been shown to inhibit the release of adhesion molecules, reactive oxygen species, interleukin (IL)-8 and neutrophil elastase, associated with neutrophils which disrupt the architecture of the lung and therefore airway function. PDE4 inhibitors influence multiple functional pathways, act on multiple immune and inflammatory pathways, and influence synthesis or release of numerous immune mediators. {J. M. Hanifin and S. C. Chan, “Atopic Dermatitis-Therapeutic Implication for New Phosphodiesterase Inhibitors,” Monocyte Dysregulation of T Cells in AACI News, 7/2 (1995); J. M. Hanifin et al., “Type 4 Phosphodiesterase Inhibitors Have clinical and In Vitro Anti-inflammatory Effects in Atopic Dermatitis,” Journal of Investigative Dermatology 107:51-56 (1996)}.

Some of the first generation of PDE4 inhibitors are effective in inhibiting PDE4 activity and alleviating a number of the inflammatory problems caused by over expression of this enzyme. However, their effectiveness is limited by side effects, particularly when used systemically, such as nausea and vomiting. {Huang et al., Curr. Opin. In Chem. Biol. 5:432-438 (2001)}. Indeed, many of the PDE4 inhibitors developed to date have been small molecule compounds with central nervous system and gastrointestinal side effects, e.g., headache, nausea/emesis, and gastric secretion.

2.3 B-RAF

B-RAF belongs to the RAF family of serine/threonine kinases. B-RAF is part of a conserved signal transduction pathway that regulates cellular responses to extracellular signals. {Wellbrock et al., Mol. Cell. Biol. 5:875-885 (2004)}. B-RAF is normally activated downstream of receptors in the cell membrane and is involved in phosphorylating and activating the protein kinase MEK, which subsequently activates the protein kinase ERK. {Niculescu-Duvas et al., J. Med. Chem. 49:407-416 (2006)}. ERK phosphorylates transcription factors such as ELK-1, regulating gene expression and controlling how cells respond to extracellular signals. Id.

B-RAF is mutated in approximately 7% of human cancers, such as melanoma (50-70%), ovarian (about 35%), thyroid (about 30%) and colorectal (about 10%) cancers. {Davies et al., Cancer Cell 2:95-98 (2003)}. The most common mutation (about 90%) is a glutamic acid for valine substitution at position 600 (V600E). {Niculescu-Duvas et al., J. Med. Chem. 49:407-416 (2006)}. The kinase activity of ^{V600E}B-RAF is elevated about 500-fold, providing cancer cells with both proliferation and survival signals and allowing them to grow as tumors in model systems. {Garnett et al., Cancer Cell 4:313-319 (2004)}. Indeed, activation of B-RAF has emerged as the most prevalent oncogenic mutation in thyroid cancer. {Salvatore et al., Clin. Can. Res. 12(5):1623-1629 (2006)}. Thus, B-RAF is an important factor in both tumor induction and maintenance and presents a new therapeutic target for human cancers. Thus, there is a need in the art for effective inhibitors of B-RAF for use as anticancer and antitumor agents.

Accordingly, there is a need in the art for effective inhibitors of TNFα, PDE4 and B-RAF.

### 3 SUMMARY

Provided herein are compounds that have activity as TNFα inhibitors, PDE4 inhibitors and/or B-RAF inhibitors, and compositions thereof. Also provided herein are methods for treating, preventing or managing certain diseases or disorders or symptoms thereof comprising administering (e.g., to a patient in need thereof) one or more compounds described herein. In particular, provided herein are methods for preventing or treating or ameliorating one or more symptoms of cancer, an inflammatory disorder, a cognition and memory disorder or an autoimmune disorder comprising administering one or more compounds described herein to a patient in need thereof.

Provided herein are compounds of formula I:

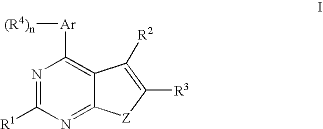

and pharmaceutically acceptable salts, solvates, hydrates, clathrates, stereoisomers, polymorphs and prodrugs thereof, wherein Ar, R¹, R², R³, R⁴, n and Z are as described herein (referred to herein as “Compound(s)”).

Also provided herein are pharmaceutical compositions comprising one or more Compounds, including pharmaceutically acceptable salts, solvates, hydrates, clathrates, stereoisomers, polymorphs and prodrugs thereof.

Also provided herein are pharmaceutical compositions comprising one or more Compounds, including pharmaceutically acceptable salts, solvates, hydrates, clathrates, stereoisomers, polymorphs and prodrugs thereof, and a pharmaceutically acceptable carrier, excipient or diluent.

Also provided herein are pharmaceutical compositions comprising one or more Compounds, or a pharmaceutically acceptable salt, solvate, hydrate, clathrate, stereoisomer, polymorph or prodrug thereof, and one or more other prophylactic or therapeutic agents, said prophylactic or therapeutic agents useful for, or having been or currently being used in the prevention, treatment or amelioration of one or more symptoms of a disease or disorder associated with or characterized by aberrant TNFα and/or PDE4 and/or B-RAF activity.

In another embodiment, provided herein are methods of preventing, treating, managing, or ameliorating septic shock, sepsis, endotoxic shock, hemodynamic shock and sepsis syndrome, post ischemic reperfusion injury, malaria, mycobacterial infection, meningitis, psoriasis, congestive heart failure, fibrotic disease, cachexia, graft rejection, cancer, auto-immune disease, opportunistic infections in AIDS, rheumatoid arthritis, rheumatoid spondylitis, osteoarthritis and other arthritic conditions, Crohn's disease, ulcerative colitis, multiple sclerosis, systemic lupus erythrematosis, ENL in leprosy, radiation damage, asthma, hyperoxic alveolar injury, stroke related memory loss, Alzheimer's disease, mild cognitive impairment, age-related cognitive decline, age-associated memory impairment, pre-senile dementia, vascular (stroke-related) dementia, Lewy body disease, fronto-temporal lobar degeneration, Pick's disease, primary progressive aphasia, corticobasal degeneration, traumatic brain injury, dementia associated with Parkinsonism, memory impairment secondary to sleep disorders, and dementia in mentally retarded adults, or one or more symptoms thereof, said methods comprising administering a prophylactically or therapeutically effective amount of a Compound, alone or in combination with a prophylactically or therapeutically effective amount of one or more other therapies.

In another embodiment, provided herein are methods for inhibiting TNFα activity in a cell expressing TNFα, comprising contacting the cell with an effective amount of a Compound or a composition thereof.

In another embodiment, provided herein are methods for inhibiting PDE4 activity in a cell expressing PDE4, comprising contacting the cell with an effective amount of a Compound or a composition thereof.

In another embodiment, provided herein are methods for inhibiting B-RAF activity in a cell expressing B-RAF, comprising contacting the cell with an effective amount of a Compound or a composition thereof.

In another embodiment, provided herein are single unit dosage forms comprising an effective amount of a Compound.

3.1 Terminology and Abbreviations

As used herein, the term “C₁₋₈alkyl” refers to a straight chain or branched hydrocarbon having from 1 to 8 carbon atoms. Representative straight-chain alkyl groups include, but are not limited to, -methyl, -ethyl, -n-propyl, -n-butyl, -n-pentyl, -n-hexyl, -n-heptyl and -n-octyl; while branched alkyl groups include, but are not limited to, -isopropyl, -sec-butyl, -isobutyl, -tert-butyl. A C₁₋₈alkyl group can be substituted or unsubstituted.

Representative O—C₁₋₈alkyl groups include, but are not limited to, —O-methyl, —O-ethyl, —O-n-propyl, —O-n-butyl, —O-isopropyl, —O-sec-butyl, —O-isobutyl and —O-tert-butyl. A O—C₁₋₈alkyl group can be substituted or unsubstituted.

Representative C₁₋₈alkylene groups include, but are not limited to, —CH₂—, —(CH₂)₂—, —(CH₂)₃—, —(CH₂)₄—, —(CH₂)₅—, —(CH₂)₆—, —(CH₂)₇— and —(CH₂)₈—. A C₁₋₈alkylene group can be substituted or unsubstituted.

Representative C₁₋₈alkylene-O—C₁₋₈alkyl groups include, but are not limited to, —CH₂—O-methyl, —CH₂—O-ethyl, —CH₂—O-n-propyl, —CH₂—O-n-butyl, —CH₂—O-isopropyl, —CH₂—O-sec-butyl, —CH₂—O-isobutyl, —CH₂—O-tert-butyl, —(CH₂)₂—O-methyl, —(CH₂)₂—O-ethyl, —(CH₂)₂—O-n-propyl, —(CH₂)₂—O-n-butyl, —(CH₂)₂—O-isopropyl, —(CH₂)₂—O-sec-butyl, —(CH₂)₂—O-isobutyl and —(CH₂)₂—O-tert-butyl. A C₁₋₈alkylene-O—C₁₋₈alkyl group can be substituted or unsubstituted.

As used herein, the term “aryl” refers to a carbocyclic aromatic group. Examples of aryl groups include, but are not limited to, phenyl, naphthyl and anthracenyl. An aryl group can be substituted or unsubstituted.

As used herein, the term “C₃₋₈cycloalkyl” refers to a 3-, 4-, 5-, 6-, 7- or 8-membered saturated or unsaturated non-aromatic carbocyclic ring. Representative C₃-C₈ cycloalkyl groups include, but are not limited to, cyclopropyl, cyclobutyl, cyclopentyl, cyclopentadienyl, cyclohexyl, cyclohexenyl, 1,3-cyclohexadienyl, 1,4-cyclohexadienyl, cycloheptyl, 1,3-cycloheptadienyl, 1,3,5-cycloheptatrienyl, cyclooctyl, and cyclooctadienyl. A C₃₋₈cycloalkyl group can be substituted or unsubstituted.

As used herein, the term “halo” means chloro, iodo, bromo, or fluoro.

As used herein, the term “heterocyclyl” refers to an aromatic or non-aromatic cycloalkyl in which one to four of the ring carbon atoms are independently replaced with a heteroatom from the group consisting of O, S and N. Representative examples of a heterocyclyl include, but are not limited to, benzofuranyl, benzothienyl, indolyl, benzopyrazolyl, coumarinyl, isoquinolinyl, morpholinyl, pyrrolyl, pyrrolidinyl, thienyl, furanyl, thiazolyl, imidazolyl, pyrazolyl, triazolyl, quinolinyl, pyrimidinyl, pyridinyl, piperizinyl, pyridonyl, pyrazinyl, pyridazinyl, isothiazolyl, isoxazolyl, (1,4)-dioxane, (1,3)-dioxolane, 4,5-dihydro-1H-imidazolyl and tetrazolyl. Heterocyclyls can also be bonded at any ring atom (i.e., at any carbon atom or heteroatom of the heterocyclic ring). A heterocyclyl group can be substituted or unsubstituted. In one embodiment, the heterocyclyl is a 3-7 membered heterocyclyl.

As used herein, the term “C₁₋₈hydroxyalkyl” refers to a C₁₋₈alkyl group as described above substituted with one or more —OH groups. Representative C₁₋₈hydroxyalkyl groups include, but are not limited to, —CH₂OH, —CH₂CH₂OH, —(CH₂)₂CH₂OH, —(CH₂)₃CH₂OH, —(CH₂)₄—CH₂OH, —(CH₂)₅CH₂OH, —CH(OH)—CH₃ and —CH₂CH(OH)CH₃.

As used herein, the term “Compound(s)” refers to any compound, including pharmaceutically acceptable salts, solvates, hydrates, clathrates, stereoisomers, polymorphs or prodrugs thereof, disclosed herein specifically or generically. In one embodiment, the Compounds are compounds of formulas I, II, IIa, IIb, IIc and those of Table 1, and pharmaceutically acceptable salts, solvates, hydrates, clathrates, stereoisomers, polymorphs or prodrugs thereof.

As used herein, the terms “therapeutically effective amount” or “effective amount” refers to the amount of a Compound which is sufficient to reduce or ameliorate the severity or duration of a disorder (e.g., a disorder associated with aberrant TNFα, PDE4 or B-RAF activity or one or more symptoms thereof), prevent the advancement of a disorder, cause regression of a disorder, prevent the recurrence, development, or onset of one or more symptoms associated with a disorder, or enhance or improve the prophylactic or therapeutic effect(s) of another therapy.

As used herein, the term “isolated” in the context of a Compound, refers to a Compound that is substantially free of chemical precursors, other chemicals when chemically synthesized or other isomers. In a specific embodiment, the Compound is 60%, 65%, 75%, 80%, 85%, 90%, 95%, or 99% free of other, different compounds (e.g., other isomers). In one embodiment, a Compound is isolated.

When the groups described herein are said to be “substituted or unsubstituted,” when substituted, they may be substituted with one or more of any substituent described herein or represented in a Compound described herein. Examples of substituents are those found in the exemplary Compounds and embodiments disclosed herein, as well as halo (e.g., chloro, iodo, bromo, or fluoro); C₁₋₈ alkyl; C₂₋₈ alkenyl; C₂₋₈ alkynyl; hydroxyl; C₁₋₈ alkoxyl; amino; nitro; thiol; thioether; imine; cyano; amido; phosphonato; phosphine; carboxyl; thiocarbonyl; sulfonyl; sulfonamide; ketone; aldehyde; ester; acetyl; acetoxy; carbamoyl; oxygen (═O); haloalkyl (e.g., trifluoromethyl); substituted aminoacyl and aminoalkyl; carbocyclic cycloalkyl, which may be monocyclic or fused or non-fused polycyclic (e.g., cyclopropyl, cyclobutyl, cyclopentyl, or cyclohexyl), or a heterocycloalkyl, which may be monocyclic or fused or non-fused polycyclic (e.g., pyrrolidinyl, piperidinyl, piperazinyl, morpholinyl, furanyl, or thiazinyl); carbocyclic or heterocyclic, monocyclic or fused or non-fused polycyclic aryl (e.g., phenyl, naphthyl, pyrrolyl, indolyl, furanyl, thienyl, imidazolyl, oxazolyl, isoxazolyl, thiazolyl, triazolyl, tetrazolyl, pyrazolyl, pyridinyl, quinolinyl, isoquinolinyl, acridinyl, pyrazinyl, pyridazinyl, pyrimidinyl, benzimidazolyl, benzothienyl, or benzofuranyl); amino (primary, secondary, or tertiary); —O-lower alkyl; —O-aryl; aryl; aryl-lower alkyl; CO₂CH₃; CONH₂; OCH₂CONH₂; NH₂; N(C₁₋₄alkyl)₂; NHC(O)C₁₋₄alkyl; SO₂NH₂; SO₂C₁₋₄alkyl; OCHF₂; CF₃; OCF₃; and such moieties may also be optionally substituted by a fused-ring structure or bridge, for example —OCH₂O— or —O-lower alkylene-O—. These substituents may optionally be further substituted with a substituent selected from such groups.

Various Compounds contain one or more chiral centers, and can exist as racemic mixtures of enantiomers, mixtures of diastereomers or enantiomerically or optically pure Compounds. Provided herein are methods comprising the use of stereomerically pure forms of such Compounds, as well as the use of mixtures of those forms. For example, mixtures comprising equal or unequal amounts of the enantiomers of a particular Compound can be used in methods and compositions provided herein. These isomers can be asymmetrically synthesized or resolved using standard techniques such as chiral columns or chiral resolving agents. {See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, Ind., 1972)}.

In a particular embodiment, a Compound is substantially free of other Compounds or active agents, such as containing less than about 0.1%, 0.5%, 1%, 2%, 3%, 4%, 5%, 10%, 15%, 20%, 25%, 30%, 35%, 40%, 45% or 50% of one or more other Compounds or active agents on a weight basis

It should be noted that if the stereochemistry of a structure or a portion of a structure is not indicated with, for example, bold or dashed lines, the structure or portion of the structure is to be interpreted as encompassing all stereoisomers of it.

As used herein, the terms “manage,” “managing,” and “management” refer to the beneficial effects that a patient derives from a Compound which does not result in a cure of the disease. In certain embodiments, a patient is administered a Compound to “manage” a disease or a symptom thereof so as to prevent the progression or worsening of the disease or symptom thereof.

As used herein, the phrase “pharmaceutically acceptable salt” refers to pharmaceutically acceptable organic or inorganic salts of a Compound. Illustrative salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. A pharmaceutically acceptable salt can involve the inclusion of another molecule such as an acetate ion, a succinate ion or other counterion. The counterion may be any organic or inorganic moiety that stabilizes the charge on the parent compound. Furthermore, a pharmaceutically acceptable salt can have more than one charged atom in its structure. Instances where multiple charged atoms are part of the pharmaceutically acceptable salt can have multiple counterions. Hence, a pharmaceutically acceptable salt can have one or more charged atoms and/or one or more counterion.

As used herein, the term “solvate” refers to an association of one or more solvent molecules and a Compound. Examples of solvents that form pharmaceutically acceptable solvates include, but are not limited to, water (i.e., hydrate), isopropanol, ethanol, methanol, DMSO, ethyl acetate, acetic acid, and ethanolamine.

As used herein, the term “pharmaceutically acceptable hydrate” refers to a Compound, or a salt thereof, that further includes a stoichiometric or non-stoichiometric amount of water bound by non-covalent intermolecular forces.

As used herein, the term “polymorph” means a particular crystalline arrangement of a Compound. Polymorphs can be obtained through the use of different work-up conditions and/or solvents. In particular, polymorphs can be prepared by recrystallization of a Compound in a particular solvent.

As used herein, the term “prodrug” means a derivative of a Compound that can hydrolyze, oxidize, or otherwise react under biological conditions (in vitro or in vivo) to provide an active compound. Examples of prodrugs include, but are not limited to, derivatives and metabolites of a Compound that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. In one embodiment, prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6^{th} ed. (Donald J. Abraham ed., 2001, Wiley) and Design and Application of Prodrugs (H. Bundgaard ed., 1985, Harwood Academic Publishers Gmfh).

As used herein, the term “clathrate” means a Compound, or a salt thereof, in the form of a crystal lattice that contains spaces (e.g., channels) that have a guest molecule (e.g., a solvent or water) trapped within, or a crystal lattice that contains one or more Compounds.

As used herein, the terms “prevent,” “preventing” and “prevention” refer to a reduction of the risk of the recurrence, onset, or development of a disorder or one or more symptoms of a disorder in a patient resulting from the administration of a Compound. In one embodiment, a disorder or symptom thereof is prevented in a patient who previously had or currently has the disorder.

As used herein, the phrase “prophylactically effective amount” refers to the amount of a therapy (e.g., prophylactic agent) which is sufficient to result in the prevention of the development, recurrence or onset of a disorder or one or more symptoms associated with a disorder (e.g., a disorder associated with aberrant TNFα, PDE4 or B-RAF activity or one or more symptoms thereof), or to enhance or improve the prophylactic effect(s) of another therapy (e.g., another prophylactic agent).

As used herein, the term “single unit dosage form” includes tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration of an effective amount of a Compound to a patient, including suspensions (e.g., aqueous or non-aqueous liquid suspensions, oil-in-water liquid emulsions, or water-in-oil liquid emulsions), solutions, and elixirs; liquid dosage forms suitable for parenteral administration of an effective amount of a Compound to a patient; and sterile solids (e.g., crystalline or amorphous solids) that can be reconstituted to provide liquid dosage forms suitable for parenteral administration of an effective amount of a Compound to a patient. Single unit dosage forms provided herein are suitable for oral, mucosal (e.g., nasal, sublingual, vaginal, buccal, or rectal), parenteral (e.g., subcutaneous, intravenous, bolus injection, intramuscular, or intraarterial), or transdermal administration of an effective amount of a Compound to a patient.

As used herein, the term “patient” refers to an animal, in one embodiment a mammal including a non-primate (e.g., a cow, pig, horse, cat, dog, rat, and mouse) and a primate (e.g., a monkey such as a cynomolgous monkey, a chimpanzee and a human), and, in one embodiment, a human.

As used herein, the terms “treat,” “treatment” and “treating” refer to the reduction or amelioration of the progression, severity and/or duration of a disorder (e.g., a disorder associated with TNFα, PDE4 or B-RAF), or the amelioration of one or more symptoms thereof resulting from the administration of a Compound.

### 4 DETAILED DESCRIPTION

Provided herein are Compounds and uses of said Compounds. Provided herein are uses of Compounds to inhibit TNFα and/or PDE4 and/or B-RAF and to treat, prevent or manage a disease, or a symptom thereof, associated with TNFα and/or PDE4 and/or B-RAF.

Also provided herein are compositions (e.g., pharmaceutical compositions) comprising an amount of a Compound effective for inhibiting TNFα and/or PDE4 and/or B-RAF or treating, preventing or managing a disease or disorder, or symptom thereof, associated with TNFα and/or PDE4 and/or B-RAF.

4.1 The Compounds

Provided herein are compounds of formula I:

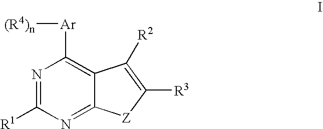

and pharmaceutically acceptable salts, solvates, hydrates, clathrates, stereoisomers, polymorphs and prodrugs thereof, wherein:

Ar is phenyl, naphthyl, pyridine or dihydrobenzofuran;

R¹ is H, C(O)R⁵R⁶, NR⁵R⁶, substituted or unsubstituted O—C₁₋₈alkyl, CN, or substituted or unsubstituted C₁₋₈alkyl;

R² is H, NH₂, substituted or unsubstituted C₁₋₈alkyl or substituted or unsubstituted aryl;

R³ is C₁₋₈hydroxyalkyl or C(O)NR⁵R⁶;

R⁴ is at each occurrence independently halo, OH, NH₂, CN, NHCN, NO₂, substituted or unsubstituted C₁₋₈alkyl, substituted or unsubstituted O—C₁₋₈alkyl, substituted or unsubstituted O—C₁₋₈alkylene-aryl, substituted or unsubstituted aryl, substituted or unsubstituted O-aryl, substituted or unsubstituted heterocyclyl, NHS(O)₂—C₁₋₈alkyl, NHC(O)—C₁₋₈alkyl, NHC(O)O—C₁₋₈alkyl, NHC(O)NHC₁₋₈alkyl, NHC(O)NHC₀₋₈alkylene-substituted or unsubstituted phenyl, NR⁵R⁶, NHC(O)NHR⁵, NHC(O)R⁵, C(O)OC₁₋₈alkyl, or C(O)NR⁵R⁶; or two adjacent R₄ groups taken together represent —OCH₂O—, —OCH₂CH₂O—, or —NH—N—; or two adjacent R₄ groups taken together with Ar form naphthalene;

R⁵ and R⁶ are at each occurrence independently H, substituted or unsubstituted C₁₋₈alkyl, substituted or unsubstituted aryl, substituted or unsubstituted heterocyclyl, substituted or unsubstituted C₁₋₈ alkylene-O—C₁₋₈alkyl, C₁₋₈alkylene-C(O)NH₂, C₁₋₈alkylene-C(O)OH, substituted or unsubstituted C₃₋₈cycloalkyl; or R⁵ and R⁶ together with the nitrogen atom to which they are attached form a 3-7 membered heterocyclyl;

Z is S, NH or O; and

n is an integer ranging from 0 to 5.

In one embodiment, compounds of formula I are those wherein Ar is phenyl.

In another embodiment, compounds of formula I are those wherein Ar is pyridine.

In one embodiment, compounds of formula I are those wherein Ar is phenyl or dihydrobenzofuran.

In another embodiment, compounds of formula I are those wherein Ar is pyridine or dihydrobenzofuran.

In another embodiment, compounds of formula I are those wherein R¹ is NR⁵R⁶, O—C₁₋₈alkyl, CN, or substituted or unsubstituted C₁₋₈alkyl.

In another embodiment, compounds of formula I are those wherein R¹ is NR⁵R⁶.

In another embodiment, compounds of formula I are those wherein R¹ is substituted O—C₁₋₈alkyl.

In another embodiment, compounds of formula I are those wherein R¹ is unsubstituted O—C₁₋₈alkyl.

In another embodiment, compounds of formula I are those wherein R¹ is C₁₋₄alkyl substituted with C₃₋₈cycloalkyl, in one embodiment cyclopropyl.

In another embodiment, compounds of formula I are those wherein R² is NH₂.

In another embodiment, compounds of formula I are those wherein R³ is C(O)NR⁵R⁶.

In another embodiment, compounds of formula I are those wherein R³ is C(O)NH₂.

In another embodiment, compounds of formula I are those wherein R³ is C(O)(3-7 membered heterocyclyl).

In another embodiment, compounds of formula I are those wherein R⁴ is at each occurrence independently halo, OH, NH₂, CN, NHCN, NO₂, substituted or unsubstituted C₁₋₈alkyl, substituted or unsubstituted aryl, substituted or unsubstituted heterocyclyl, NHS(O)₂—C₁₋₈alkyl, NHC(O)—C₁₋₈alkyl, NHC(O)O—C₁₋₈alkyl, NC(O)NC₁₋₈alkyl, NC(O)NC₀₋₈alkylene-substituted or unsubstituted phenyl, C(O)OC₁₋₈alkyl, or C(O)NR⁵R⁶; or two adjacent R₄ groups taken together represent —OCH₂O—, —OCH₂CH₂O—, or —NH—N—; or two adjacent R₄ groups taken together with Ar form naphthalene.

In another embodiment, compounds of formula I are those wherein at least one of R⁵ and R⁶ is C₁₋₄hydroxyalkyl.

In another embodiment, compounds of formula I are those wherein Z is S and R⁵ and R⁶ are at each occurrence independently substituted or unsubstituted C₁₋₈alkyl, substituted or unsubstituted aryl, substituted or unsubstituted heterocyclyl, substituted or unsubstituted C₁₋₈alkylene-O—C₁₋₈alkyl, substituted or unsubstituted C₃₋₈cycloalkyl; or R⁵ and R⁶ together with the nitrogen atom to which they are attached form a 3-7 membered heterocyclyl.

In another embodiment, compounds of formula I are those wherein Z is S and R² is NH₂.

In another embodiment, compounds of formula I are those wherein Z is S and R⁵ and R⁶ together with the nitrogen atom to which they are attached form a 3-7 membered heterocyclyl.

In another embodiment, compounds of formula I are those wherein n is an integer ranging from 1 to 5.

In another embodiment, provided herein are compounds of formula Ia:

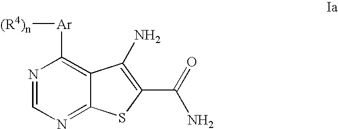

and pharmaceutically acceptable salts, solvates, hydrates, clathrates, stereoisomers, polymorphs and prodrugs thereof, wherein:

Ar is phenyl, pyridine or dihydrobenzofuran;

R⁴ is at each occurrence independently halo, OH, NO₂, substituted or unsubstituted O—C₁₋₈alkyl, substituted or unsubstituted O—C₁₋₈alkylene-aryl, NHC(O)NHC₀₋₈alkylene-substituted or unsubstituted phenyl, NR⁵R⁶, NHC(O)NHR⁵, NHC(O)R⁵;

R⁵ and R⁶ are at each occurrence independently H, substituted or unsubstituted C₁₋₈alkyl, substituted or unsubstituted aryl, substituted or unsubstituted heterocyclyl, substituted or unsubstituted C₁₋₈alkylene-O—C₁₋₈alkyl, C₁₋₈alkylene-C(O)NH₂, C₁₋₈alkylene-C(O)OH, substituted or unsubstituted C₃₋₈cycloalkyl; or R⁵ and R⁶ together with the nitrogen atom to which they are attached form a 3-7 membered heterocyclyl; and

n is an integer ranging from 0 to 5.

In another embodiment, provided herein are compounds of formula II:

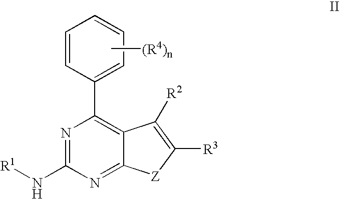

and pharmaceutically acceptable salts, solvates, hydrates, clathrates, stereoisomers, polymorphs and prodrugs thereof, wherein:

R¹ is H, C(O)NR⁵R⁶, substituted or unsubstituted C₁₋₈alkyl, substituted or unsubstituted C₁₋₈alkylene-O—C₁₋₈alkyl or substituted or unsubstituted C₃₋₈cycloalkyl;

R² is H, NH₂, substituted or unsubstituted C₁₋₈alkyl or substituted or unsubstituted aryl;

R³ is C₁₋₈hydroxyalkyl or C(O)NR⁵R⁶;

R⁴ is at each occurrence independently halo, substituted or unsubstituted C₁₋₈alkyl, substituted or unsubstituted O—C₁₋₈alkyl, substituted or unsubstituted O—C₁₋₈alkylene-aryl, substituted or unsubstituted aryl, substituted or unsubstituted O-aryl, substituted or unsubstituted heterocyclyl or C(O)NR⁵R⁶; or two adjacent R₄ groups taken together represent —OCH₂O— or —OCH₂CH₂O—;

R⁵ and R⁶ are at each occurrence independently H, substituted or unsubstituted C₁₋₈alkyl, C₁₋₈alkylene-C(O)NH₂, C₁₋₈alkylene-C(O)OH, substituted or unsubstituted aryl or substituted or unsubstituted heterocyclyl; or R⁵ and R⁶ together with the nitrogen atom to which they are attached form a 3-7 membered heterocyclyl;

Z is S, NH or O; and

n is an integer ranging from 0 to 5.

In one embodiment, compounds of formula II are those wherein R¹ is H.

In another embodiment, compounds of formula II are those wherein R¹ is C₁₋₄alkyl, for example methyl, ethyl, isopropyl or methyl substituted with cyclopropyl.

In another embodiment, compounds of formula II are those wherein R¹ is C₃₋₈cycloalkyl, for example cyclopropyl.

In another embodiment, compounds of formula II are those wherein R¹ is C₁₋₄alkylene-O—C₁₋₄alkyl, for example —CH₂CH₂—O—CH₃.

In another embodiment, compounds of formula II are those wherein R¹ is C₁₋₄hydroxyalkyl.

In another embodiment, compounds of formula II are those wherein R² is NH₂.

In another embodiment, compounds of formula II are those wherein R² is H.

In another embodiment, compounds of formula II are those wherein R³ is C(O)NR⁵R⁶, for example C(O)NH₂, C(O)NHCH₃, C(O)N(CH₃)₂, C(O)NHC(CH₃)₃, C(O)-morpholine, C(O)-methylpiperazine or C(O)NH-phenyl.

In another embodiment, compounds of formula II are those wherein R³ is C(O)(3-7 membered heterocyclyl).

In another embodiment, compounds of formula II are those wherein R³ is C₁₋₄hydroxyalkyl, for example CH₂OH or C(CH₃)₂OH.

In another embodiment, compounds of formula II are those wherein R⁴ is halo, for example chloro or fluoro.

In another embodiment, compounds of formula II are those wherein R⁴ is C₁₋₄alkyl, for example methyl or trifluoromethyl.

In another embodiment, compounds of formula II are those wherein R⁴ is aryl, for example phenyl.

In another embodiment, compounds of formula II are those wherein R⁴ is heterocyclyl, for example pyridine, pyrrolidine or morpholine.

In another embodiment, compounds of formula II are those wherein R⁴ is C(O)NR⁵R⁶, for example C(O)NHCH₃.

In another embodiment, compounds of formula II are those wherein two adjacent R₄ groups taken together represent —OCH₂O—.

In another embodiment, compounds of formula II are those wherein R⁴ is at each occurrence independently halo, OH, NH₂, CN, NHCN, NO₂, substituted or unsubstituted C₁₋₈alkyl, substituted or unsubstituted aryl, substituted or unsubstituted heterocyclyl, NHS(O)₂—C₁₋₈alkyl, NHC(O)—C₁₋₈alkyl, NHC(O)O—C₁₋₈alkyl, NC(O)NC₁₋₈alkyl, NC(O)NC₀₋₈alkylene-substituted or unsubstituted phenyl, C(O)OC₁₋₈alkyl, or C(O)NR⁵R⁶; or two adjacent R₄ groups taken together represent —OCH₂O—, —OCH₂CH₂O—, or —NH [symbol] N—; or two adjacent R₄ groups taken together with Ar form naphthalene.

In another embodiment, compounds of formula II are those wherein R⁵ and R⁶ are both H.

In another embodiment, compounds of formula II are those wherein R⁵ and R⁶ are both methyl.

In another embodiment, compounds of formula II are those wherein one of R⁵ and R⁶ is H and the other is C₁₋₄alkyl, for example methyl or tert-butyl.

In another embodiment, compounds of formula II are those wherein one of R⁵ and R⁶ is H and the other is aryl, for example phenyl.

In another embodiment, compounds of formula II are those wherein R⁵ and R⁶ together with the nitrogen atom to which they are attached form a 3-7 membered heterocyclyl, for example morpholine or methylpiperazine.

In another embodiment, compounds of formula II are those wherein n is 0.

In another embodiment, compounds of formula II are those wherein n is 1.

In another embodiment, compounds of formula II are those wherein n is 2.

In another embodiment, compounds of formula II are those wherein n is 3.

In another embodiment, compounds of formula II are those wherein n is 4.

In another embodiment, compounds of formula II are those wherein n is 5.

In another embodiment, compounds of formula II are those wherein n is an integer ranging from 1 to 5.

In another embodiment, compounds of formula II are those wherein n is 1 and R₄ is halo, for example chloro or fluoro.

In another embodiment, compounds of formula II are those wherein n is 1 and R₄ is C₁₋₄alkyl, for example trifluoromethyl.

In another embodiment, compounds of formula II are those wherein n is 1 and R₄ is aryl, for example phenyl.

In another embodiment, compounds of formula II are those wherein n is 1 and R₄ is heterocyclyl, for example pyridine, pyrrolidine or morpholine.

In another embodiment, compounds of formula II are those wherein n is 1 and R₄ is C(O)NR⁵R⁶, for example C(O)NHCH₃.

In another embodiment, compounds of formula II are those wherein n is 2 and R₄ is halo, for example both occurrences of R₄ are chloro.

In another embodiment, compounds of formula II are those wherein n is 2 and R₄ is C₁₋₄alkyl, for example both occurrences of R₄ are methyl or trifluoromethyl.

In another embodiment, compounds of formula II are those wherein n is 2 wherein one R₄ is halo, for example fluoro, and the other R₄ is C₁₋₄alkyl, for example methyl.

In another embodiment, compounds of formula II are those wherein n is 2 wherein two adjacent R₄ groups taken together represent —OCH₂O—.

In another embodiment, Z is S or NH.

In another embodiment, Z is S or O.

In another embodiment, Z is O or NH.

In one embodiment, provided herein are compounds of formula IIa:

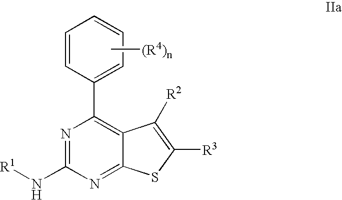

and pharmaceutically acceptable salts, solvates, hydrates, clathrates, stereoisomers, polymorphs and prodrugs thereof, wherein:

R¹ is H, C(O)NR⁵R⁶, substituted or unsubstituted C₁₋₈alkyl, substituted or unsubstituted C₁₋₈alkylene-O—C₁₋₈alkyl or substituted or unsubstituted C₃₋₈cycloalkyl;

R² is H, NH₂, substituted or unsubstituted C₁₋₈alkyl or substituted or unsubstituted aryl;

R³ is C₁₋₈hydroxyalkyl or C(O)NR⁵R⁶;

R⁴ is at each occurrence independently halo, substituted or unsubstituted C₁₋₈alkyl, substituted or unsubstituted O—C₁₋₈alkyl, substituted or unsubstituted O—C₁₋₈alkylene-aryl, substituted or unsubstituted aryl, substituted or unsubstituted O-aryl, substituted or unsubstituted heterocyclyl or C(O)NR⁵R⁶; or two adjacent R₄ groups taken together represent —OCH₂O— or —OCH₂CH₂O—;

R⁵ and R⁶ are at each occurrence independently H, substituted or unsubstituted C₁₋₈alkyl, C₁₋₈alkylene-C(O)NH₂, C₁₋₈alkylene-C(O)OH, substituted or unsubstituted aryl or substituted or unsubstituted heterocyclyl; or R⁵ and R⁶ together with the nitrogen atom to which they are attached form a 3-7 membered heterocyclyl; and

n is an integer ranging from 0 to 5.

In a particular embodiment, R¹ is substituted or unsubstituted C₁₋₄alkyl, substituted or unsubstituted C₁₋₄alkylene-O—C₁₋₄alkyl or substituted or unsubstituted C₃₋₈cycloalkyl.

In another embodiment, R² is NH₂, substituted or unsubstituted C₁₋₄alkyl or substituted or unsubstituted aryl.

In another embodiment, R² is NH₂.

In another embodiment, R⁴ is at each occurrence independently halo, OH, NH₂, CN, NHCN, NO₂, substituted or unsubstituted C₁₋₈alkyl, substituted or unsubstituted aryl, substituted or unsubstituted heterocyclyl, NHS(O)₂—C₁₋₈alkyl, NHC(O)—C₁₋₈alkyl, NHC(O)O—C₁₋₈alkyl, NC(O)NC₁₋₈alkyl, NC(O)NC₀₋₈alkylene-substituted or unsubstituted phenyl, C(O)OC₁₋₈alkyl, or C(O)NR⁵R⁶; or two adjacent R₄ groups taken together represent —OCH₂O, —OCH₂CH₂O—, or —NH [symbol] N—; or two adjacent R₄ groups taken together with Ar form naphthalene.

In another embodiment, R⁵ and R⁶ together with the nitrogen atom to which they are attached form a benzofuranyl, benzothienyl, indolyl, benzopyrazolyl, coumarinyl, isoquinolinyl, pyrrolyl, thienyl, furanyl, thiazolyl, imidazolyl, pyrazolyl, triazolyl, quinolinyl, pyrimidinyl, pyridinyl, piperizinyl, pyridonyl, pyrazinyl, pyridazinyl, isothiazolyl, isoxazolyl, (1,4)-dioxane, (1,3)-dioxolane, 4,5-dihydro-1H-imidazolyl or tetrazolyl ring.

In another embodiment, n is an integer ranging from 1 to 5.

In one embodiment, provided herein are compounds of formula IIb:

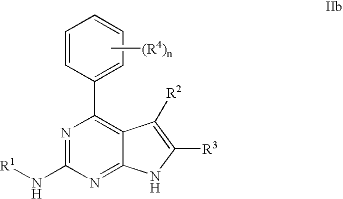

and pharmaceutically acceptable salts, solvates, hydrates, clathrates, stereoisomers, polymorphs and prodrugs thereof, wherein:

R¹ is H, C(O)NR⁵R⁶, substituted or unsubstituted C₁₋₈alkyl, substituted or unsubstituted C₁₋₈alkylene-O—C₁₋₈alkyl or substituted or unsubstituted C₃₋₈cycloalkyl;

R² is H, NH₂, substituted or unsubstituted C₁₋₈alkyl or substituted or unsubstituted aryl;

R³ is C₁₋₈hydroxyalkyl or C(O)NR⁵R⁶;

R⁴ is at each occurrence independently halo, substituted or unsubstituted C₁₋₈alkyl, substituted or unsubstituted O—C₁₋₈alkyl, substituted or unsubstituted O—C₁₋₈alkylene-aryl, substituted or unsubstituted aryl, substituted or unsubstituted O-aryl, substituted or unsubstituted heterocyclyl or C(O)NR⁵R⁶; or two adjacent R₄ groups taken together represent —OCH₂O— or —OCH₂CH₂O—;

R⁵ and R⁶ are at each occurrence independently H, substituted or unsubstituted C₁₋₄alkyl, C₁₋₈alkylene-C(O)NH₂, C₁₋₈alkylene-C(O)OH, substituted or unsubstituted aryl or substituted or unsubstituted heterocyclyl; or R⁵ and R⁶ together with the nitrogen atom to which they are attached form a 3-7 membered heterocyclyl; and

n is an integer ranging from 0 to 5.

In a particular embodiment, R¹ is substituted or unsubstituted C₁₋₄alkyl, substituted or unsubstituted C₁₋₄alkylene-O—C₁₋₄alkyl or substituted or unsubstituted C₃₋₈cycloalkyl.

In another embodiment, R² is NH₂, substituted or unsubstituted C₁₋₄alkyl or substituted or unsubstituted aryl.

In another embodiment, R² is NH₂.

In another embodiment, R⁴ is at each occurrence independently halo, OH, NH₂, CN, NHCN, NO₂, substituted or unsubstituted C₁₋₈alkyl, substituted or unsubstituted aryl, substituted or unsubstituted heterocyclyl, NHS(O)₂—C₁₋₈alkyl, NHC(O)—C₁₋₈alkyl, NHC(O)O—C₁₋₈alkyl, NC(O)NC₁₋₈alkyl, NC(O)NC₀₋₈alkylene-substituted or unsubstituted phenyl, NR⁵R⁶, NHC(O)NHR⁵, NHC(O)R⁵, C(O)OC₁₋₈alkyl, or C(O)NR⁵R⁶; or two adjacent R₄ groups taken together represent —OCH₂O, —OCH₂CH₂O—, or —NH [symbol] N—; or two adjacent R₄ groups taken together with Ar form naphthalene.

In another embodiment, R⁵ and R⁶ together with the nitrogen atom to which they are attached form a benzofuranyl, benzothienyl, indolyl, benzopyrazolyl, coumarinyl, isoquinolinyl, pyrrolyl, thienyl, furanyl, thiazolyl, imidazolyl, pyrazolyl, triazolyl, quinolinyl, pyrimidinyl, pyridinyl, piperizinyl, pyridonyl, pyrazinyl, pyridazinyl, isothiazolyl, isoxazolyl, (1,4)-dioxane, (1,3)-dioxolane, 4,5-dihydro-1H-imidazolyl or tetrazolyl ring.

In another embodiment, n is an integer ranging from 1 to 5.

In one embodiment, provided herein are compounds of formula IIc:

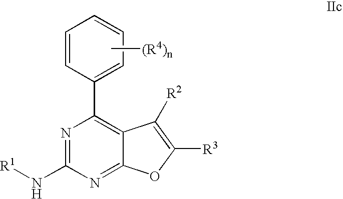

and pharmaceutically acceptable salts, solvates, hydrates, clathrates, stereoisomers, polymorphs and prodrugs thereof, wherein:

R¹ is H, C(O)NR⁵R⁶, substituted or unsubstituted C₁₋₈alkyl, substituted or unsubstituted C₁₋₈alkylene-O—C₁₋₈alkyl or substituted or unsubstituted C₃₋₈cycloalkyl;

R² is H, NH₂, substituted or unsubstituted C₁₋₈alkyl or substituted or unsubstituted aryl;

R³ is C₁₋₈hydroxyalkyl or C(O)NR⁵R⁶;

R⁴ is at each occurrence independently halo, substituted or unsubstituted C₁₋₈alkyl, substituted or unsubstituted O—C₁₋₈alkyl, substituted or unsubstituted O—C₁₋₈alkylene-aryl, substituted or unsubstituted aryl, substituted or unsubstituted O-aryl, substituted or unsubstituted heterocyclyl or C(O)NR⁵R⁶; or two adjacent R₄ groups taken together represent —OCH₂O— or —OCH₂CH₂O—;

R⁵ and R⁶ are at each occurrence independently H, substituted or unsubstituted C₁₋₄alkyl, C₁₋₈alkylene-C(O)NH₂, C₁₋₈alkylene-C(O)OH, substituted or unsubstituted aryl or substituted or unsubstituted heterocyclyl; or R⁵ and R⁶ together with the nitrogen atom to which they are attached form a 3-7 membered heterocyclyl; and

n is an integer ranging from 0 to 5.

In a particular embodiment, R¹ is substituted or unsubstituted C₁₋₄alkyl, substituted or unsubstituted C₁₋₄alkylene-O—C₁₋₄alkyl or substituted or unsubstituted C₃₋₈cycloalkyl.

In another embodiment, R² is NH₂, substituted or unsubstituted C₁₋₄alkyl or substituted or unsubstituted aryl.

In another embodiment, R² is NH₂.

In another embodiment, R⁴ is at each occurrence independently halo, OH, NH₂, CN, NHCN, NO₂, substituted or unsubstituted C₁₋₈alkyl, substituted or unsubstituted aryl, substituted or unsubstituted heterocyclyl, NHS(O)₂—C₁₋₈alkyl, NHC(O)—C₁₋₈alkyl, NHC(O)O—C₁₋₈alkyl, NHC(O)NHC₁₋₈alkyl, NHC(O)NHC₀₋₈alkylene-substituted or unsubstituted phenyl, NR⁵R⁶, NHC(O)NHR⁵, NHC(O)R⁵, C(O)OC₁₋₈alkyl, or C(O)NR⁵R⁶; or two adjacent R₄ groups taken together represent —OCH₂O, —OCH₂CH₂O—, or —NH—N—; or two adjacent R₄ groups taken together with Ar form naphthalene.

In another embodiment, R⁵ and R⁶ together with the nitrogen atom to which they are attached form a benzofuranyl, benzothienyl, indolyl, benzopyrazolyl, coumarinyl, isoquinolinyl, pyrrolyl, thienyl, furanyl, thiazolyl, imidazolyl, pyrazolyl, triazolyl, quinolinyl, pyrimidinyl, pyridinyl, piperizinyl, pyridonyl, pyrazinyl, pyridazinyl, isothiazolyl, isoxazolyl, (1,4)-dioxane, (1,3)-dioxolane, 4,5-dihydro-1H-imidazolyl or tetrazolyl ring.

In another embodiment, n is an integer ranging from 1 to 5.

Illustrative examples of the Compounds include those set forth in Table 1, below, and pharmaceutically acceptable salts, solvates or hydrates thereof.

TABLE 1

| Comp. | Structure |
|---|---|
| 1 | 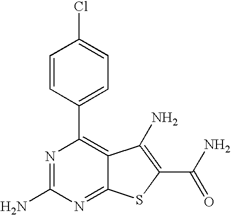 |
| 2 | 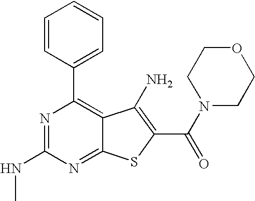 |
| 3 | 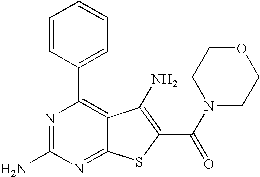 |
| 4 | 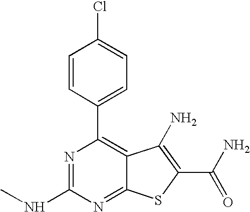 |
| 5 | 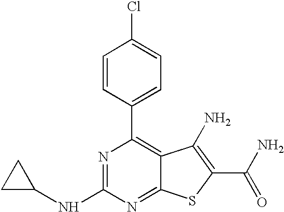 |
| 6 | 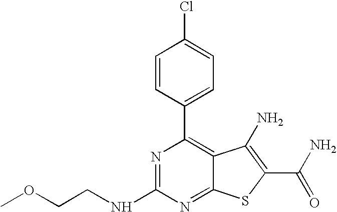 |
| 7 | 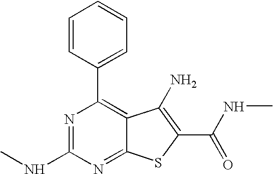 |
| 8 | 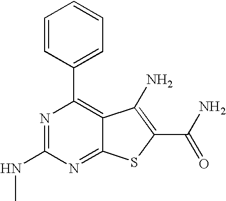 |
| 9 | 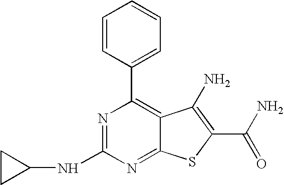 |
| 10 | 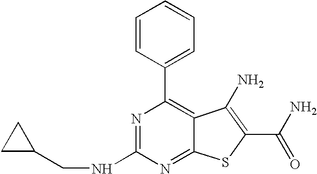 |
| 11 | 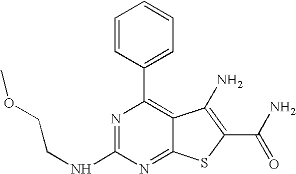 |
| 12 | 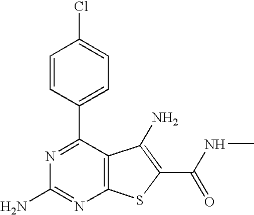 |
| 13 | 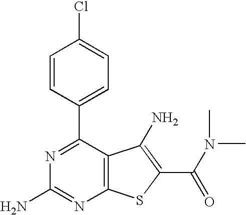 |
| 14 | 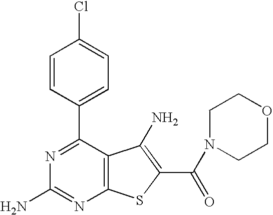 |
| 15 | 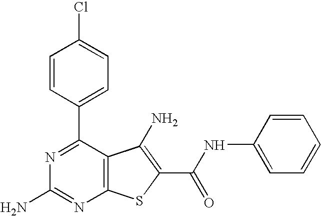 |
| 16 | 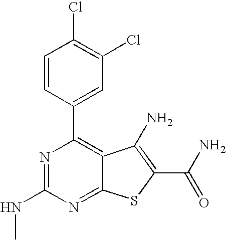 |
| 17 | 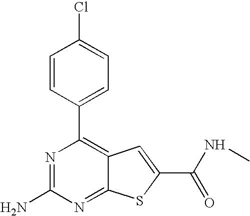 |
| 18 | 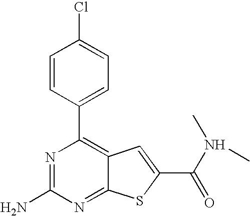 |
| 19 | 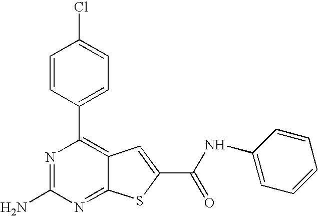 |
| 20 | 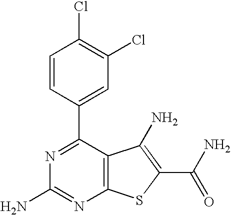 |
| 21 | 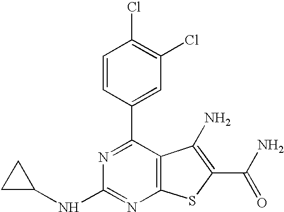 |
| 22 | 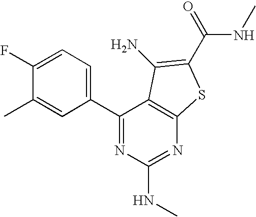 |
| 23 | 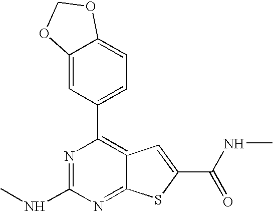 |
| 24 | 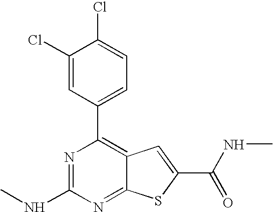 |
| 25 | 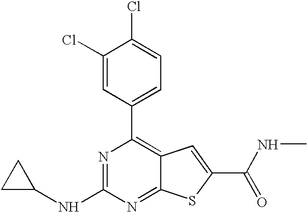 |
| 26 | 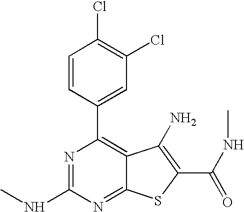 |
| 27 | 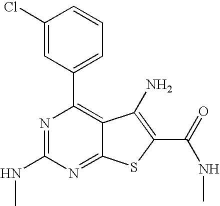 |
| 28 | 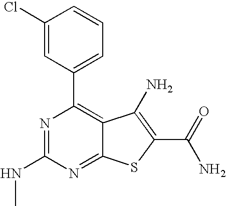 |
| 29 | 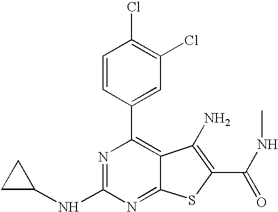 |
| 30 | 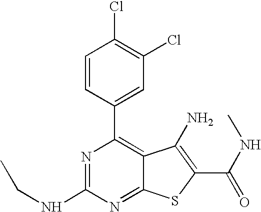 |
| 31 | 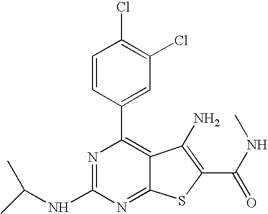 |
| 32 | 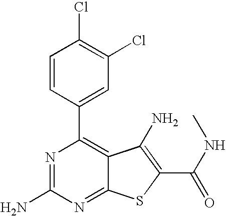 |
| 33 | 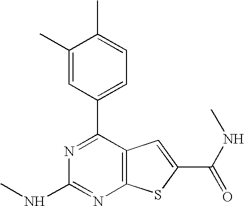 |
| 34 | 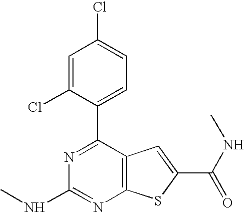 |
| 35 | 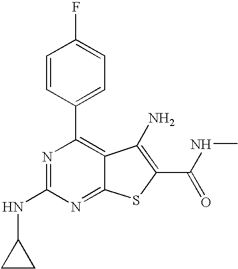 |
| 36 | 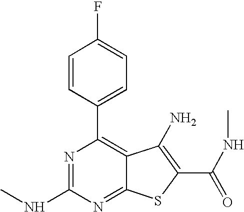 |
| 37 | 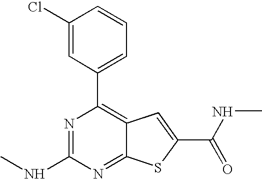 |
| 38 | 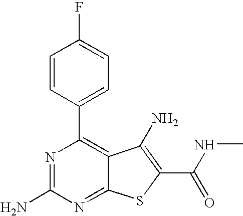 |
| 39 | 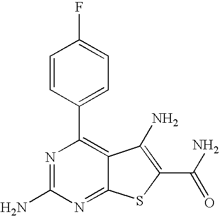 |
| 40 | 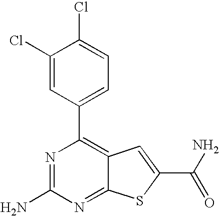 |
| 41 | 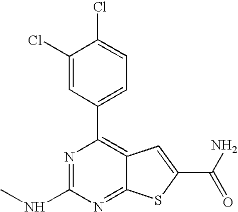 |
| 42 | 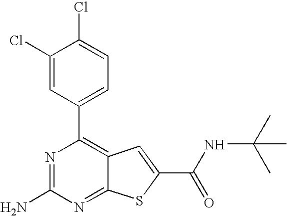 |
| 43 | 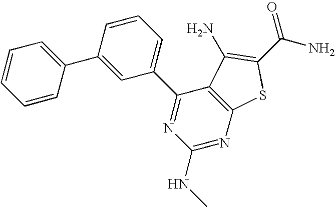 |
| 44 | 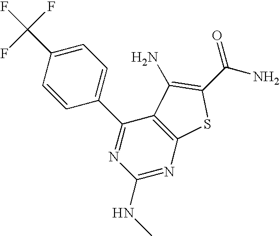 |
| 45 | 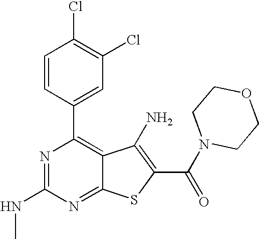 |
| 46 | 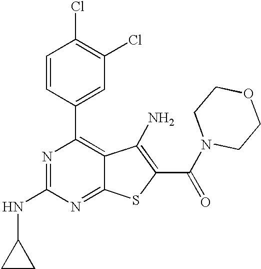 |
| 47 | 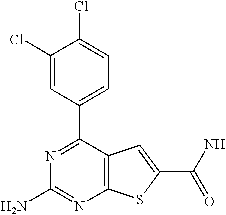 |
| 48 | 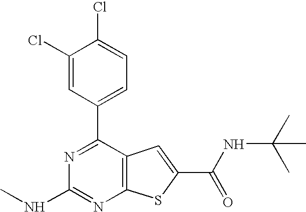 |
| 49 | 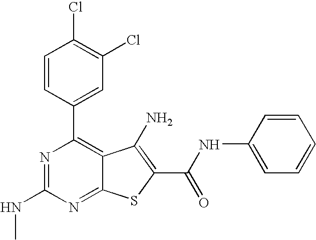 |
| 50 | 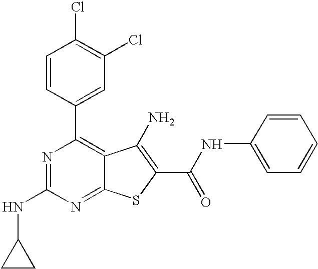 |
| 51 | 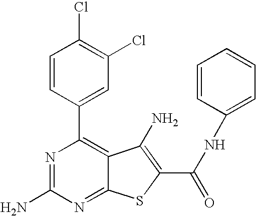 |
| 52 | 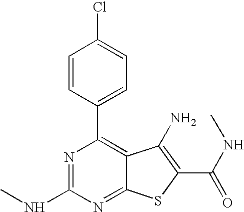 |
| 53 | 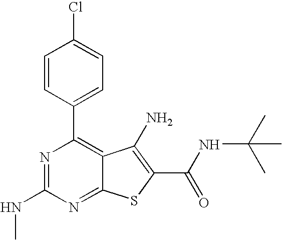 |
| 54 | 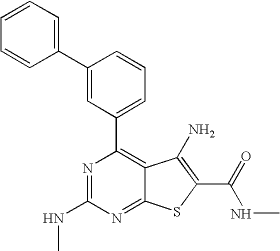 |
| 55 | 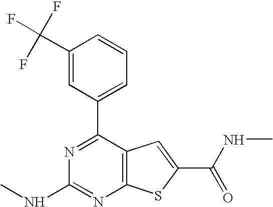 |
| 56 | 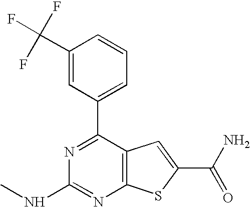 |
| 57 | 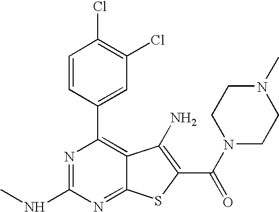 |
| 58 | 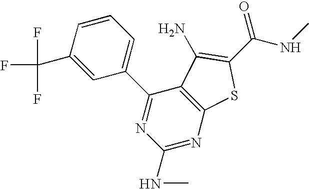 |
| 59 | 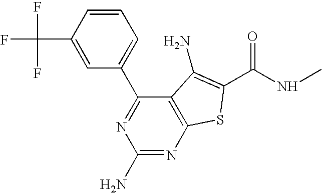 |
| 60 | 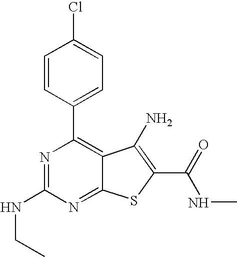 |
| 61 | 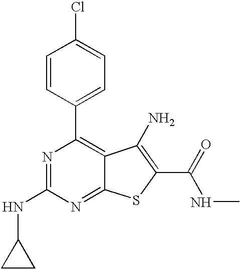 |
| 62 | 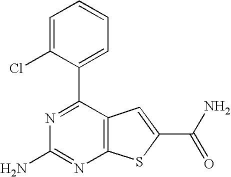 |
| 63 | 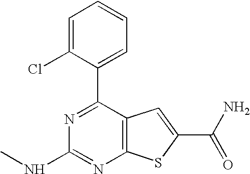 |
| 64 | 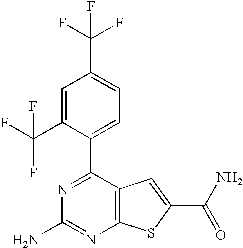 |
| 65 | 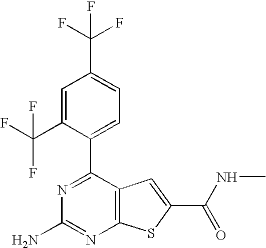 |
| 66 | 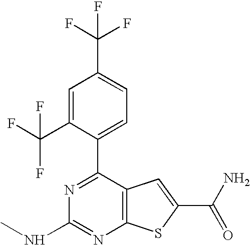 |
| 67 | 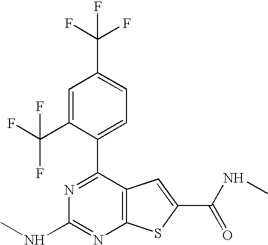 |
| 68 | 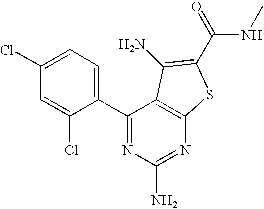 |
| 69 | 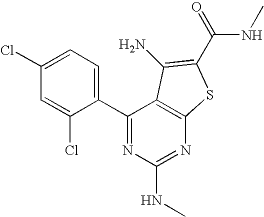 |
| 70 | 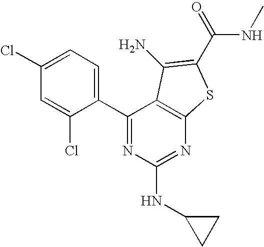 |
| 71 | 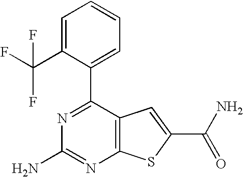 |
| 72 | 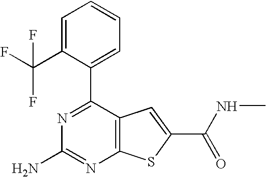 |
| 73 | 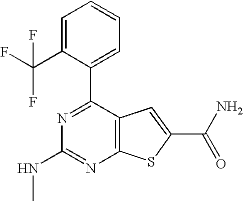 |
| 74 | 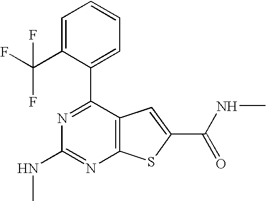 |
| 75 | 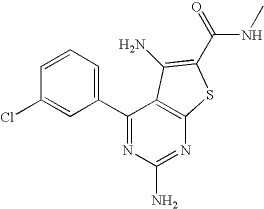 |
| 76 | 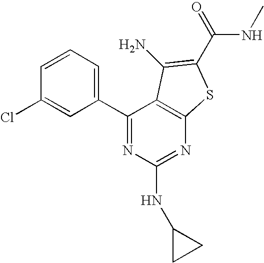 |
| 77 | 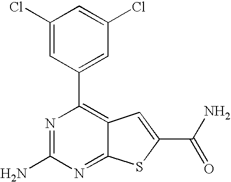 |
| 78 | 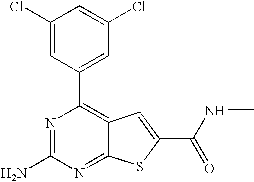 |
| 79 | 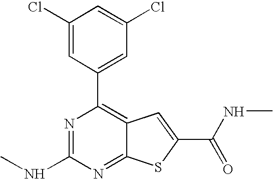 |
| 80 | 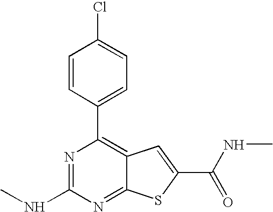 |
| 81 | 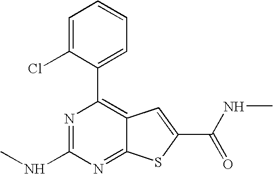 |
| 82 | 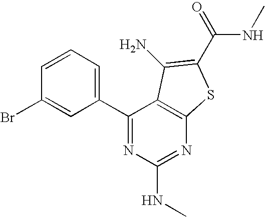 |
| 83 | 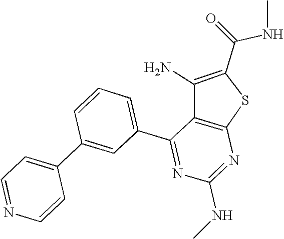 |
| 84 | 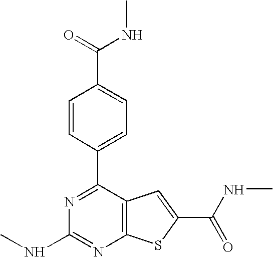 |
| 85 | 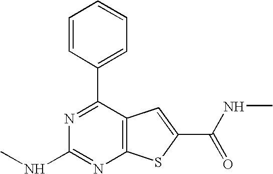 |
| 86 | 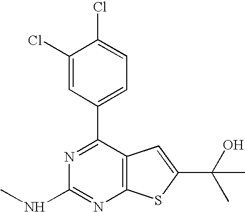 |
| 87 | 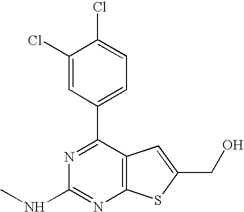 |
| 88 | 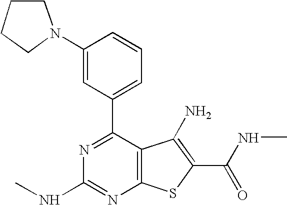 |
| 89 | 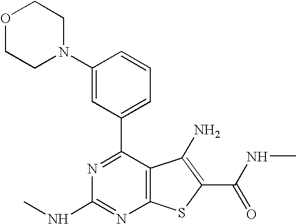 |
| 90 | 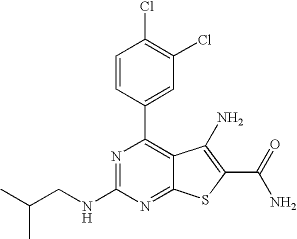 |
| 91 | 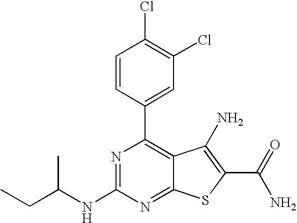 |
| 92 | 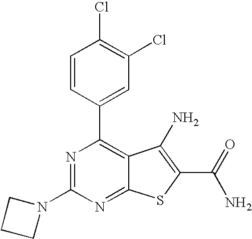 |
| 93 | 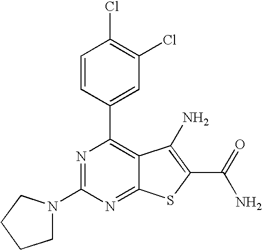 |
| 94 | 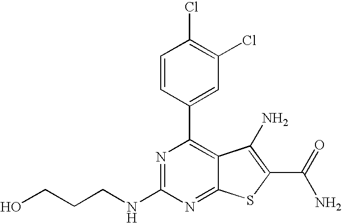 |
| 95 | 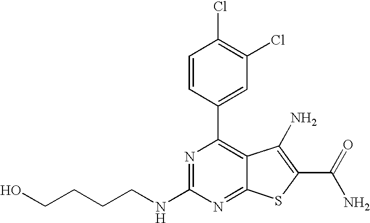 |
| 96 | 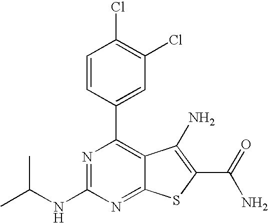 |
| 97 | 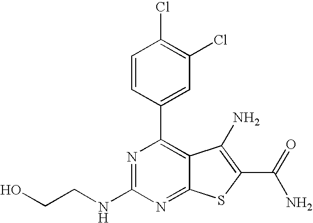 |
| 98 | 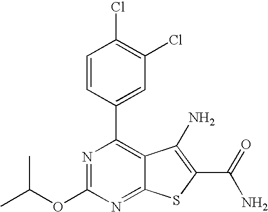 |
| 99 | 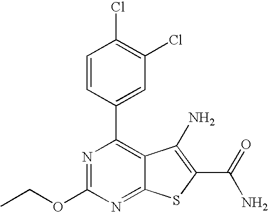 |
| 100 | 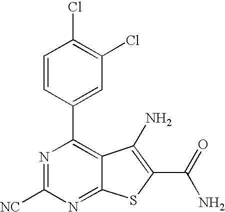 |
| 101 | 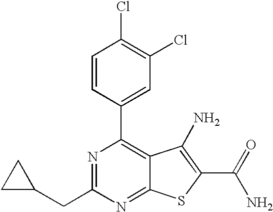 |
| 102 | 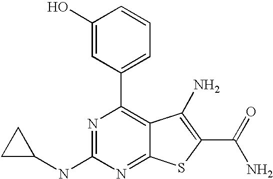 |
| 103 | 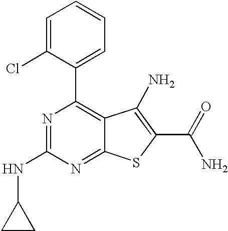 |
| 104 | 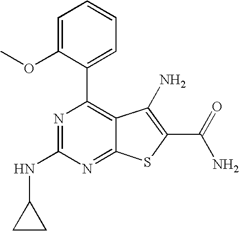 |
| 105 | 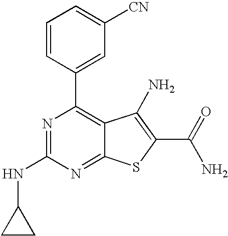 |
| 106 | 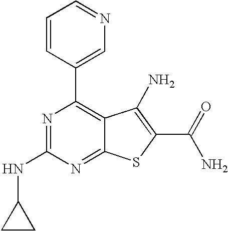 |
| 107 | 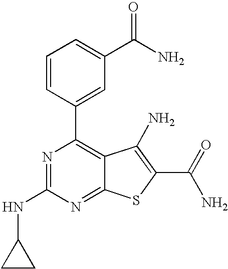 |
| 108 | 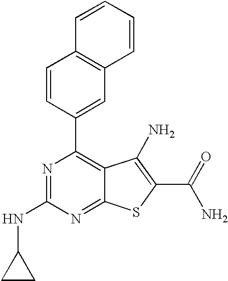 |
| 109 | 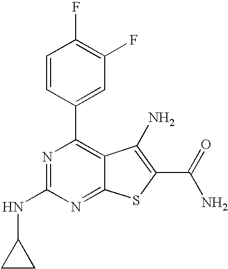 |
| 110 | 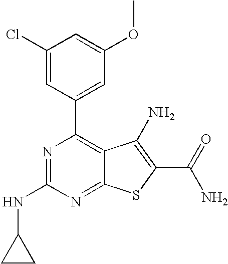 |
| 111 | 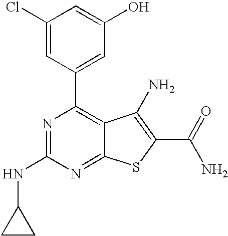 |
| 112 | 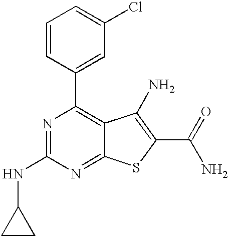 |
| 113 | 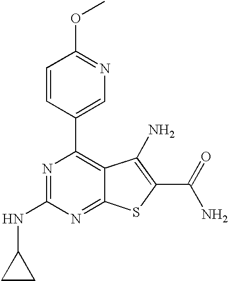 |
| 114 | 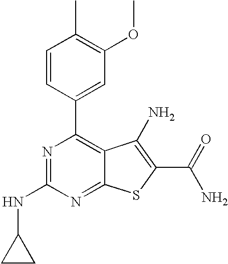 |
| 115 | 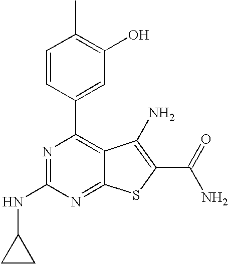 |
| 116 | 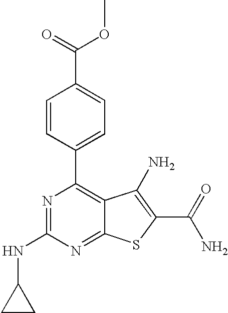 |
| 117 | 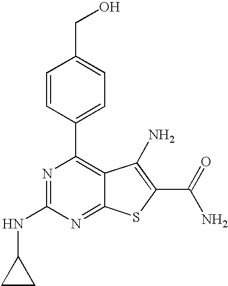 |
| 118 | 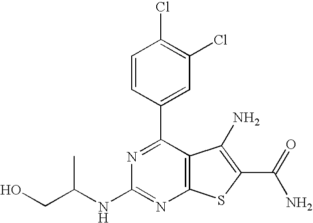 |
| 119 | 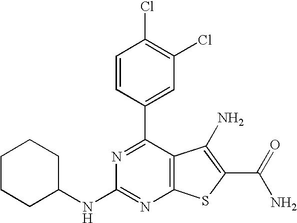 |
| 120 | 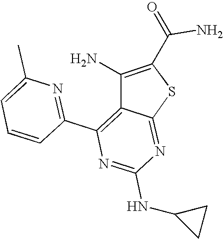 |
| 121 | 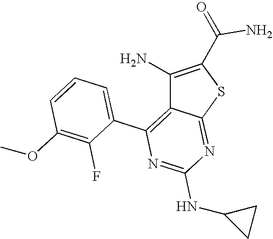 |
| 122 | 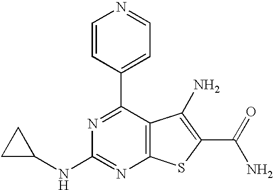 |
| 123 | 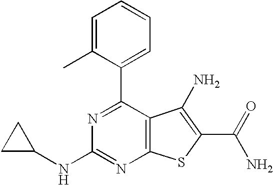 |
| 124 | 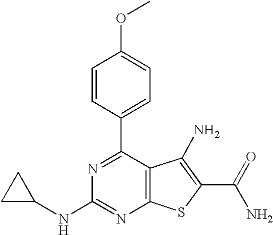 |
| 125 | 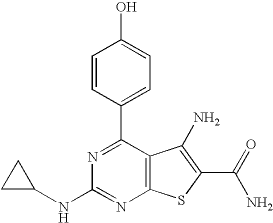 |
| 126 | 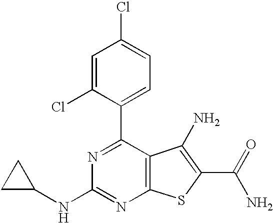 |
| 127 | 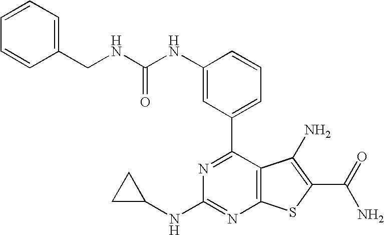 |
| 128 | 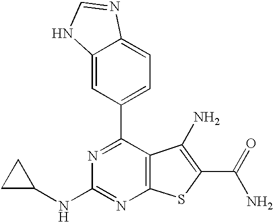 |
| 129 | 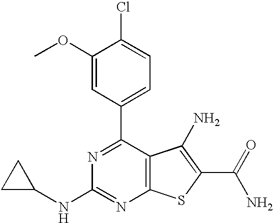 |
| 130 | 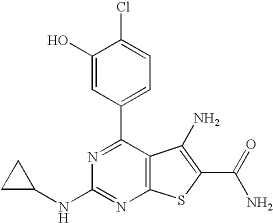 |
| 131 | 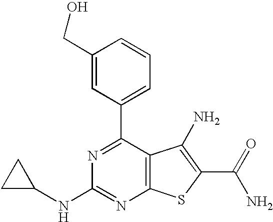 |
| 132 | 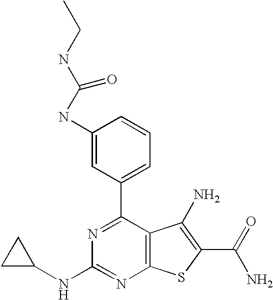 |
| 133 | 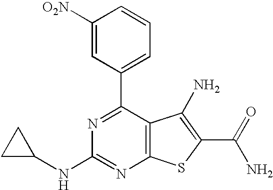 |
| 134 | 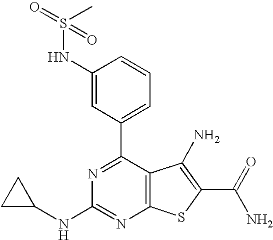 |
| 135 | 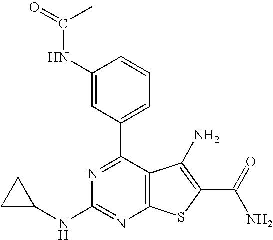 |
| 136 | 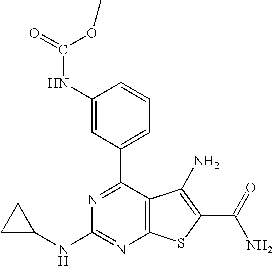 |
| 137 | 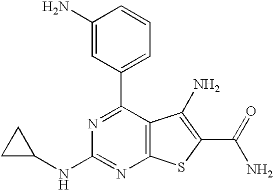 |
| 138 | 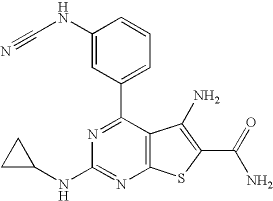 |
| 139 | 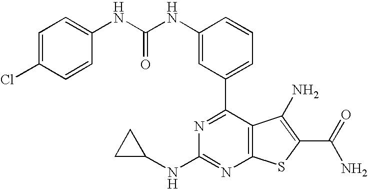 |
| 140 | 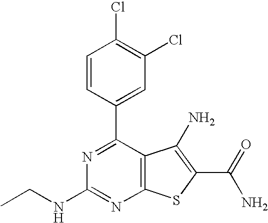 |
| 141 | 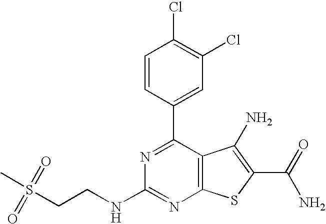 |
| 142 | 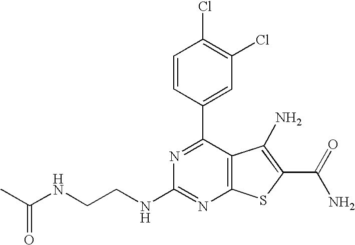 |
| 143 | 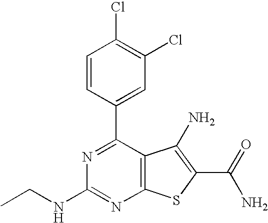 |
| 144 | 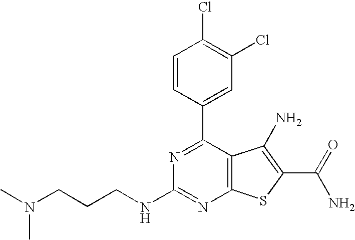 |
| 145 | 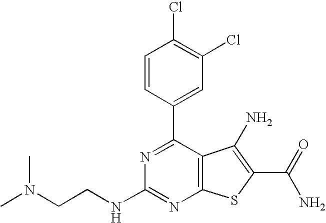 |
| 146 | 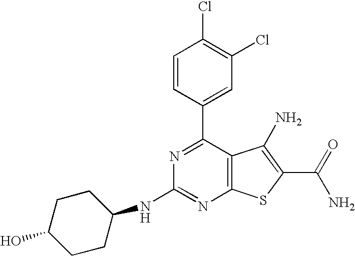 |
| 147 | 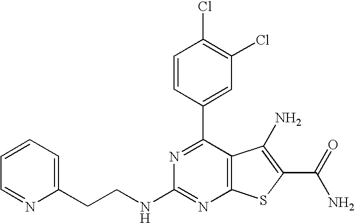 |
| 148 | 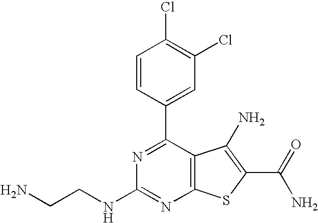 |
| 149 | 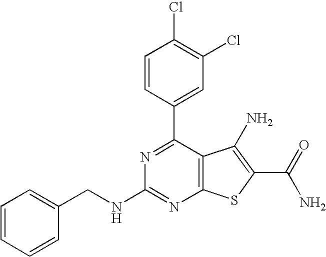 |
| 150 | 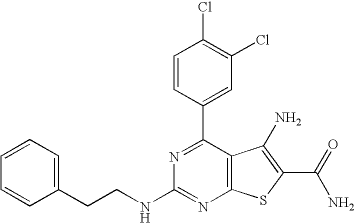 |
| 151 | 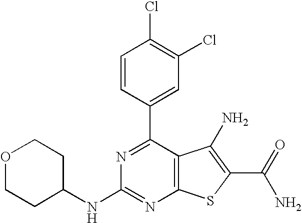 |
| 152 | 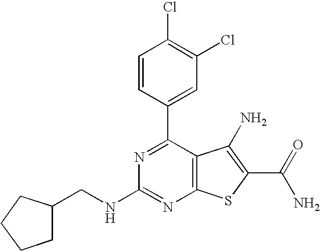 |
| 153 | 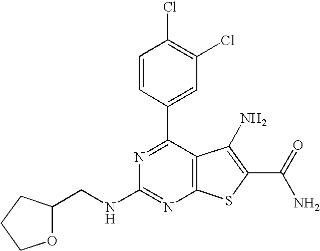 |
| 154 | 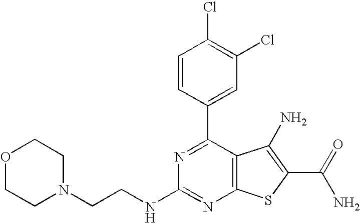 |
| 155 | 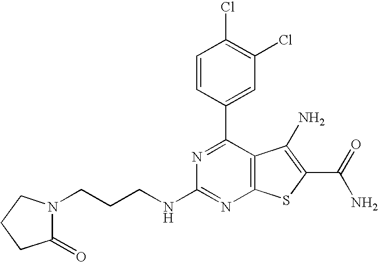 |
| 156 | 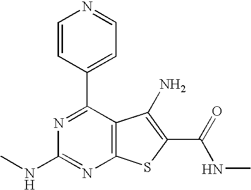 |
| 157 | 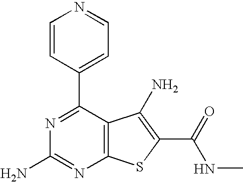 |
| 158 | 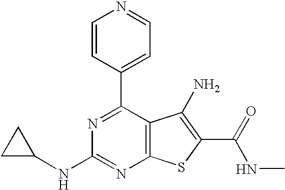 |
| 159 | 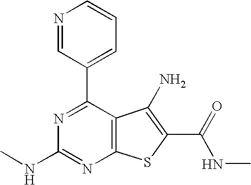 |
| 160 | 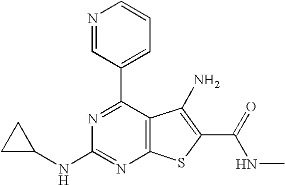 |
| 161 | 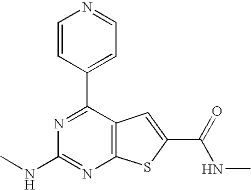 |
| 162 | 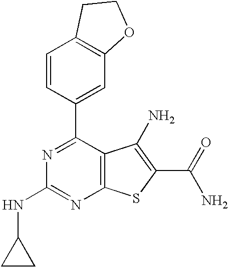 |
| 163 | 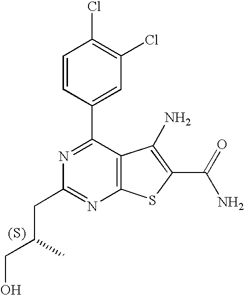 |
| 164 | 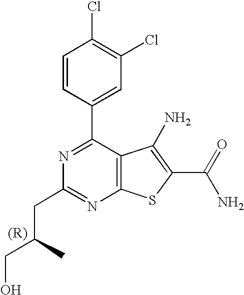 |
| 165 | 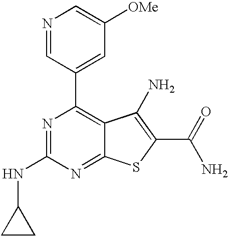 |
| 166 | 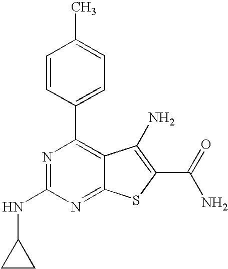 |
| 167 | 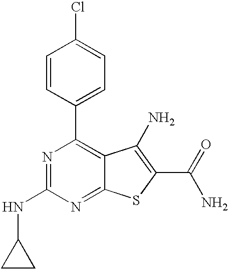 |
| 168 | 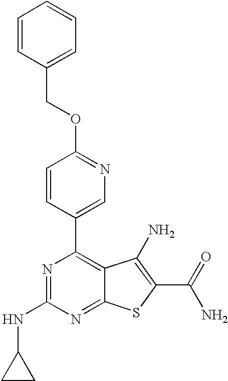 |
| 169 | 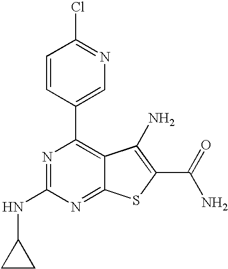 |
| 170 | 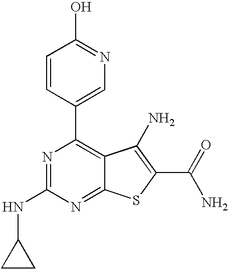 |
| 171 | 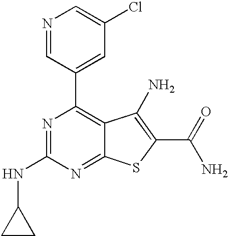 |
| 172 | 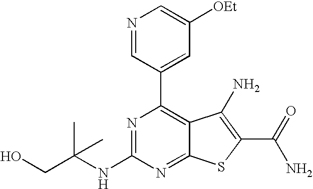 |
| 173 | 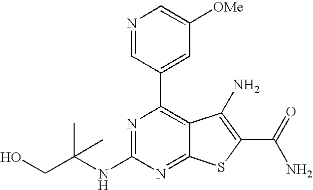 |
| 174 | 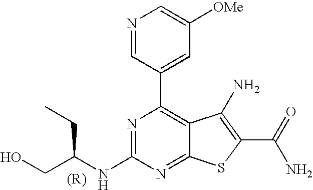 |
| 175 | 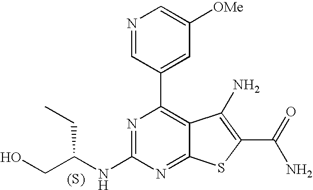 |
| 176 | 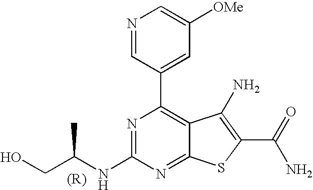 |
| 177 | 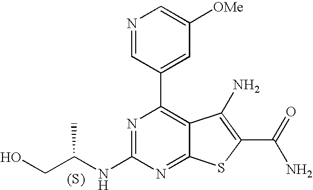 |
| 178 | 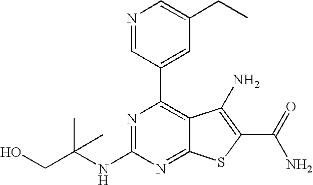 |
| 179 | 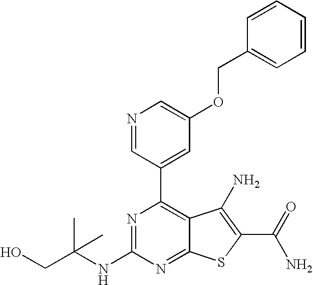 |
| 180 | 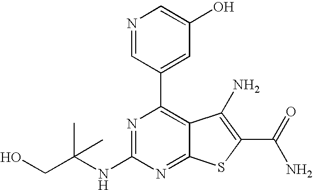 |
| 181 | 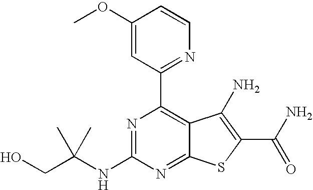 |
| 182 | 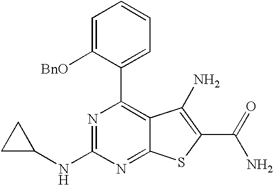 |
| 183 | 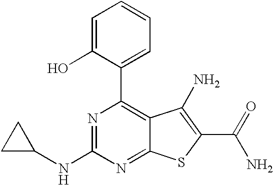 |
| 184 | 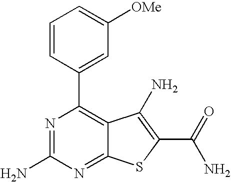 |
| 185 | 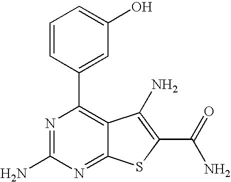 |
| 186 | 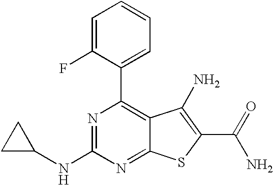 |
| 187 | 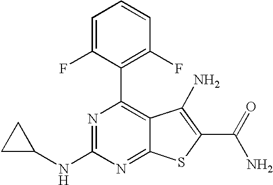 |
| 188 | 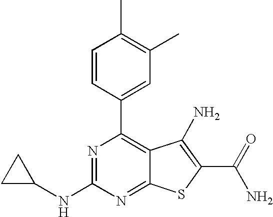 |
| 189 | 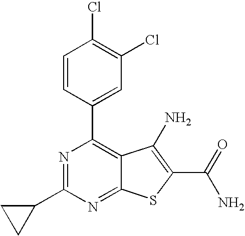 |
| 190 | 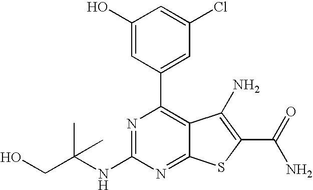 |
| 191 | 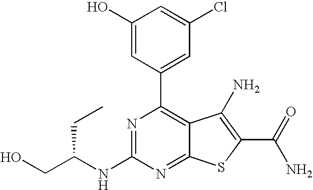 |
| 192 | 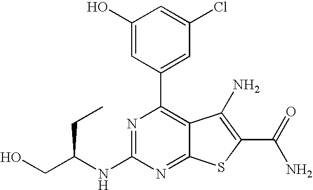 |
| 193 | 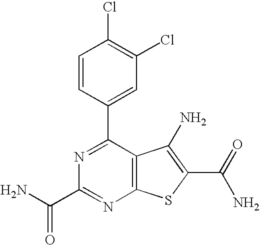 |
| 194 | 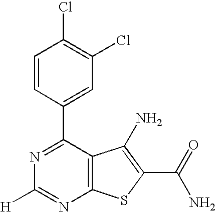 |
| 195 | 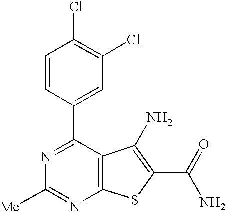 |
| 196 | 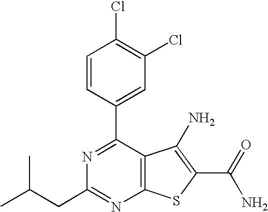 |
| 197 | 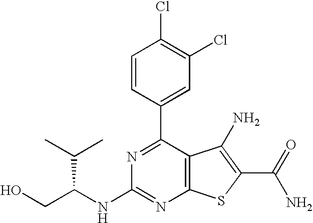 |
| 198 | 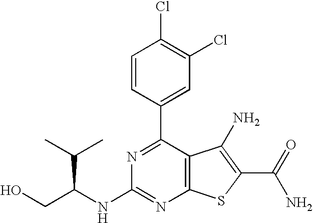 |
| 199 | 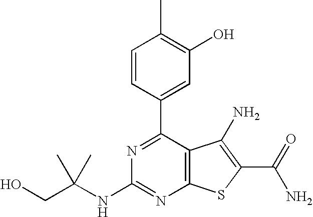 |
| 200 | 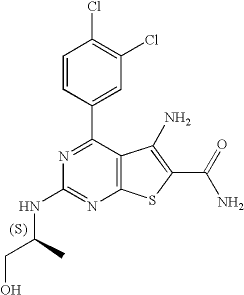 |
| 201 | 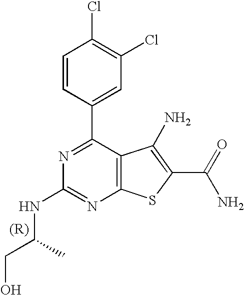 |
| 202 | 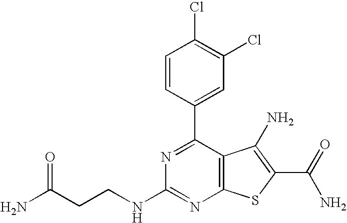 |
| 203 | 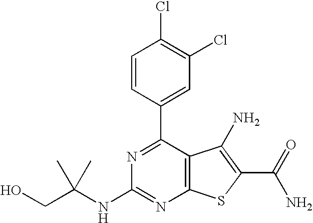 |
| 204 | 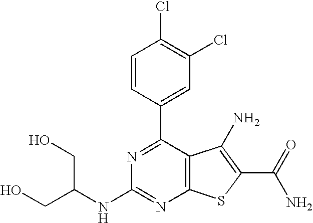 |
| 205 | 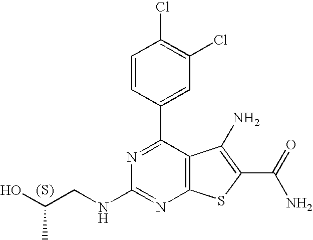 |
| 206 | 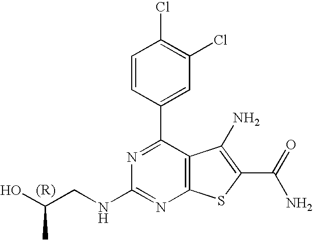 |
| 207 | 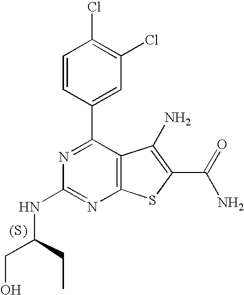 |
| 208 | 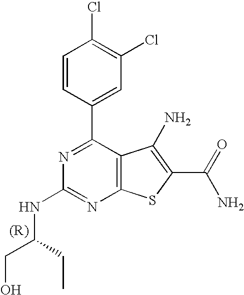 |
| 209 | 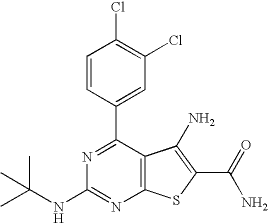 |
| 210 | 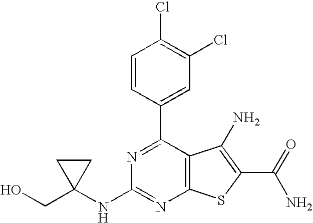 |
| 211 | 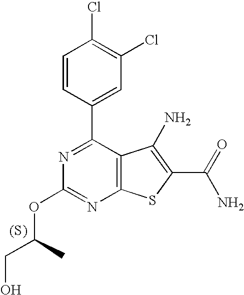 |
| 212 | 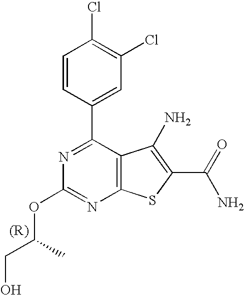 |
| 213 | 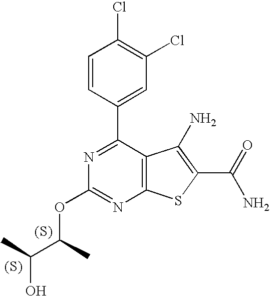 |
| 214 | 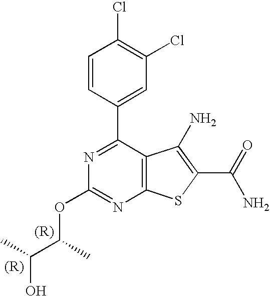 |
| 215 | 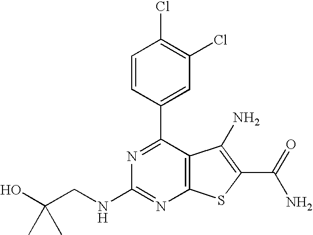 |
| 216 | 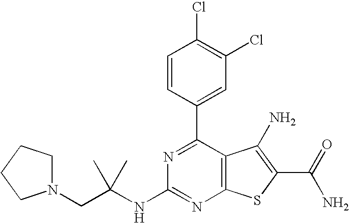 |
| 217 | 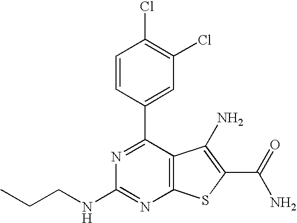 |
| 218 | 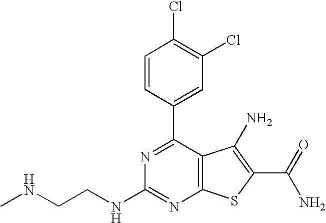 |
| 219 | 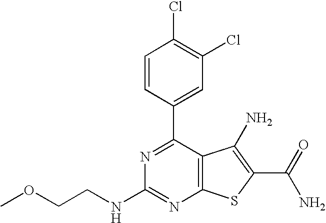 |
| 220 | 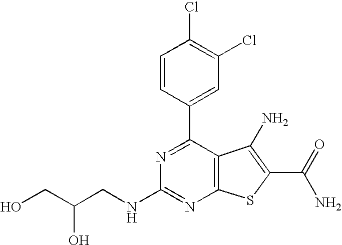 |
| 221 | 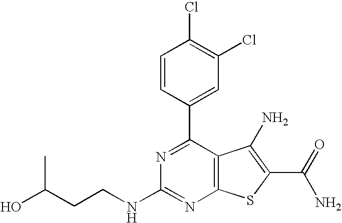 |
| 222 | 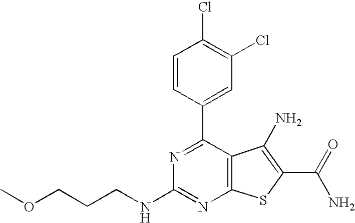 |
| 223 | 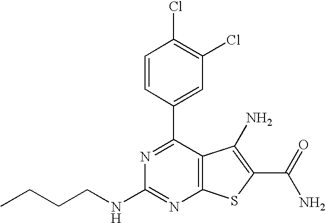 |
| 224 | 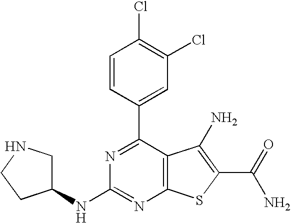 |
| 225 | 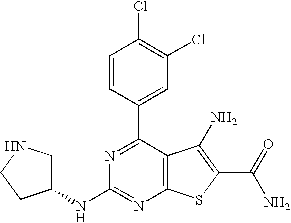 |
| 226 | 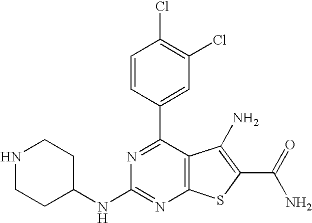 |
| 227 | 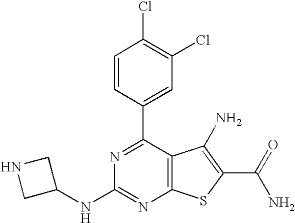 |
| 228 | 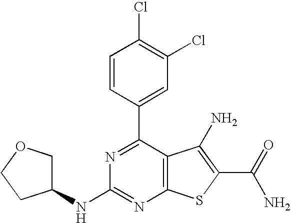 |
| 229 | 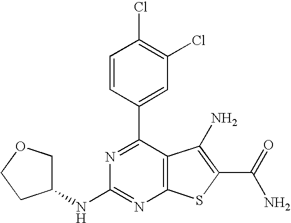 |
| 230 | 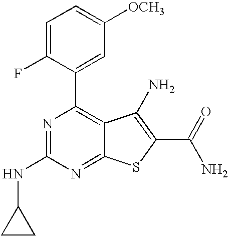 |
| 231 | 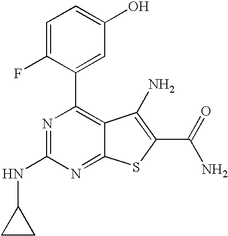 |
| 232 | 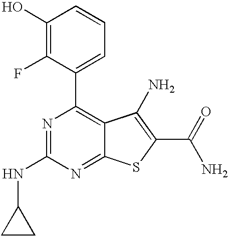 |
| 233 | 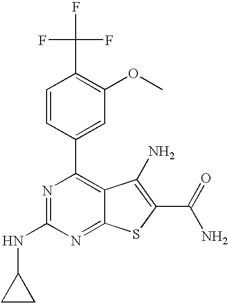 |
| 234 | 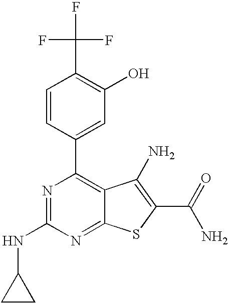 |
| 235 | 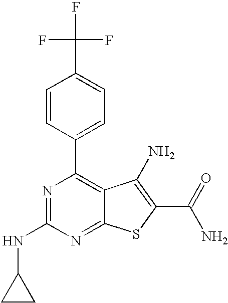 |
| 236 | 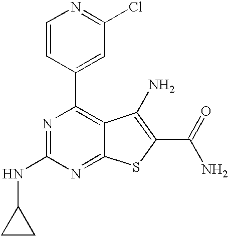 |
| 237 | 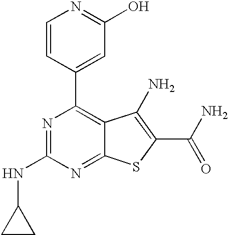 |
| 238 | 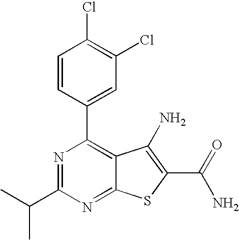 |
| 239 | 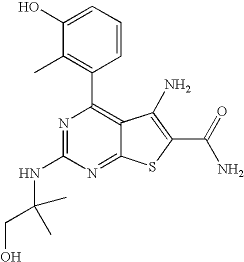 |
| 240 | 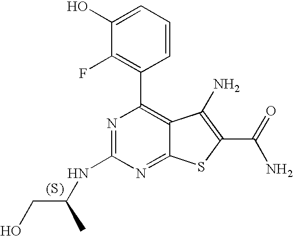 |
| 241 | 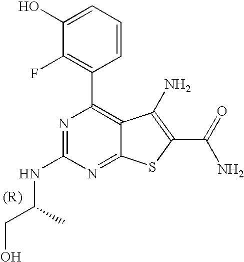 |
| 242 | 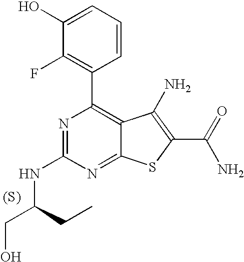 |
| 243 | 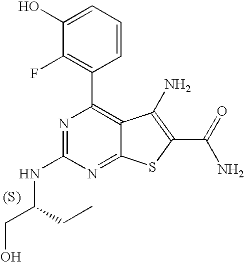 |
| 244 | 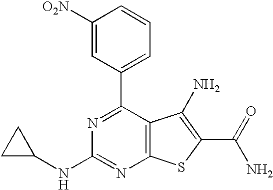 |
| 245 | 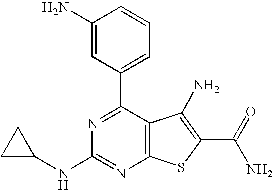 |
| 246 | 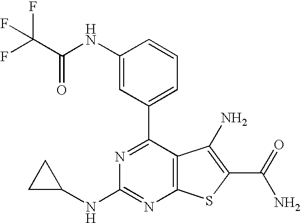 |
| 247 | 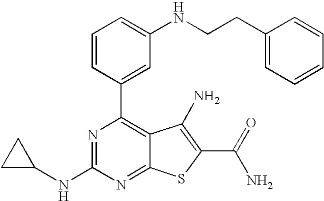 |
| 248 | 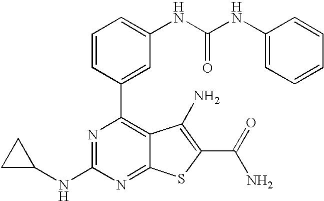 |
| 249 | 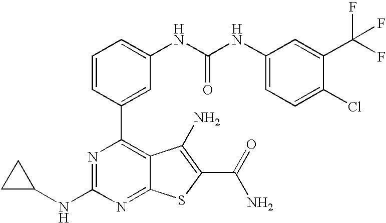 |
| 250 | 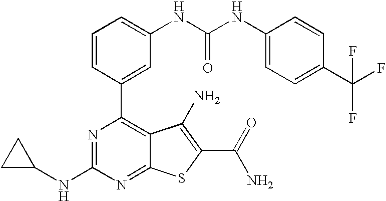 |
| 251 | 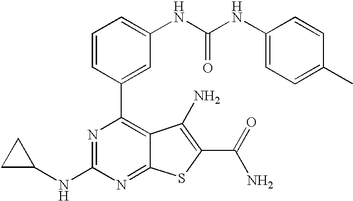 |
| 252 | 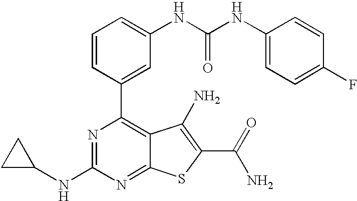 |
| 253 | 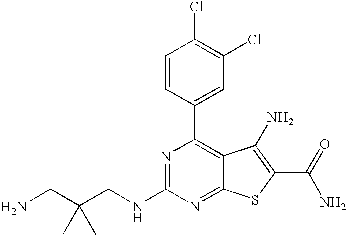 |
| 254 | 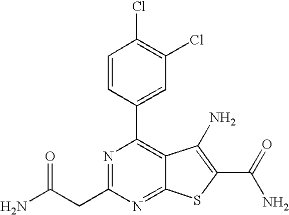 |
| 255 | 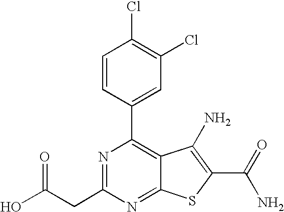 |
| 256 | 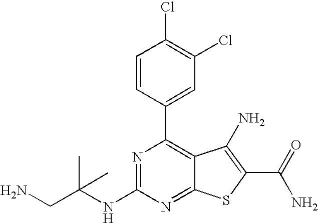 |
| 257 | 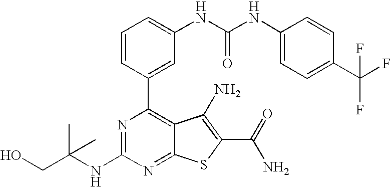 |
| 258 | 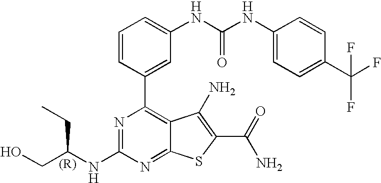 |
| 259 | 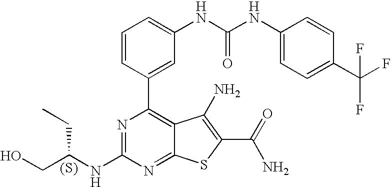 |
| 260 | 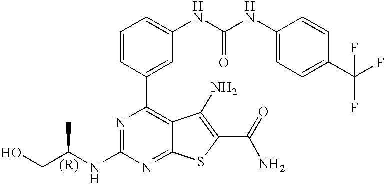 |
| 261 | 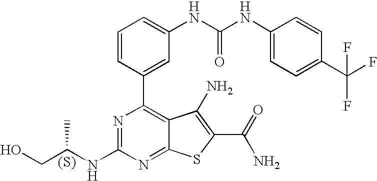 |
| 262 | 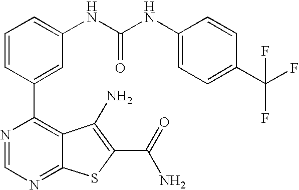 |
| 263 | 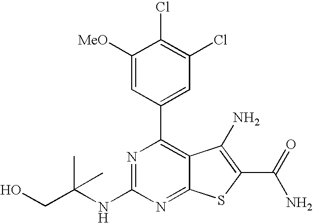 |
| 264 | 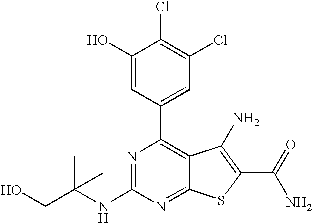 |
| 265 | 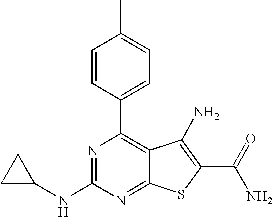 |
| 266 | 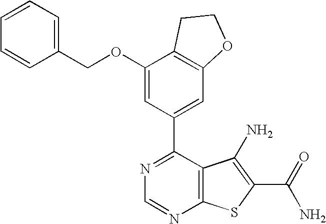 |
| 267 | 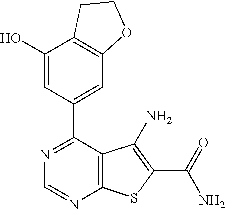 |
| 268 | 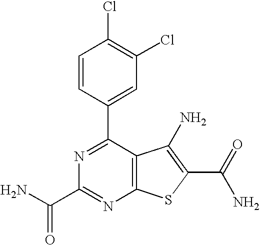 |
| 269 | 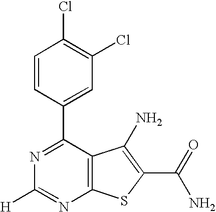 |
| 270 | 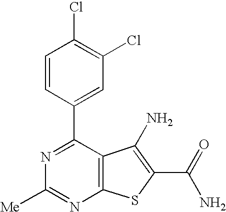 |
| 271 | 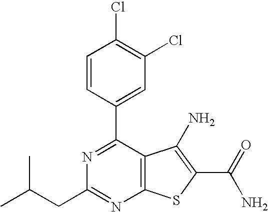 |
| 272 | 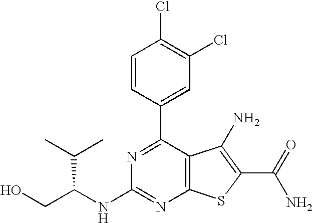 |
| 273 | 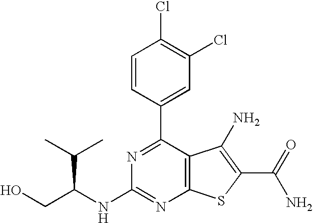 |
| 274 | 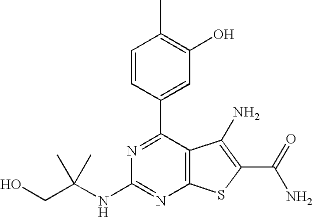 |
| 275 | 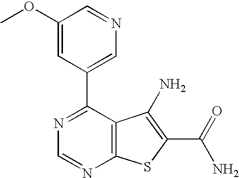 |
| 276 | 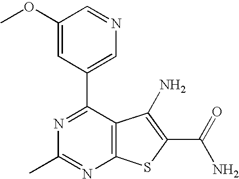 |
| 277 | 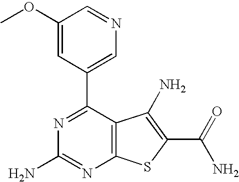 |
| 278 | 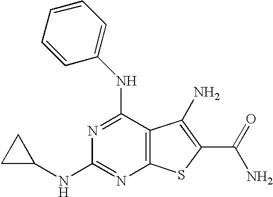 |
| 279 | 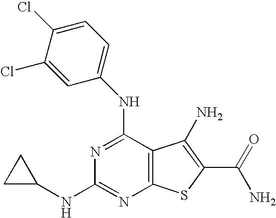 |
| 280 | 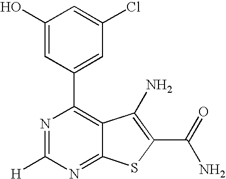 |
| 281 | 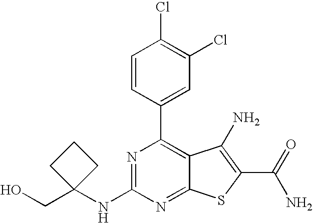 |
| 282 | 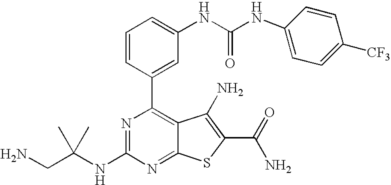 |
| 283 | 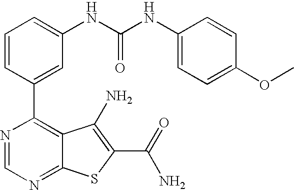 |
| 284 | 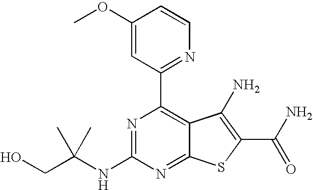 |
| 285 | 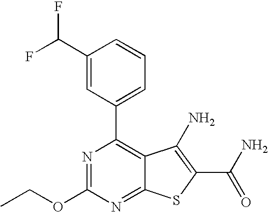 |
| 286 | 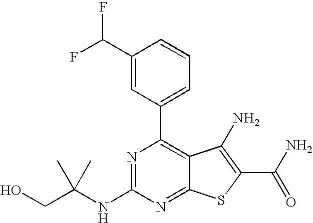 |
| 287 | 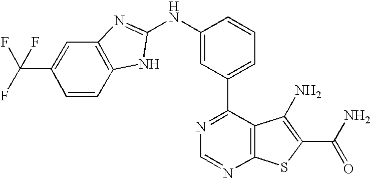 |
| 288 | 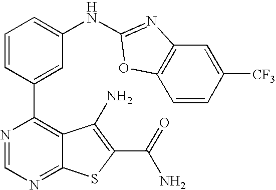 |
| 289 | 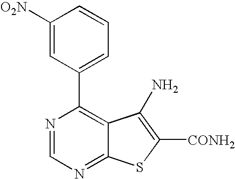 |
| 290 | 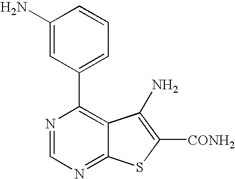 |
| 291 | 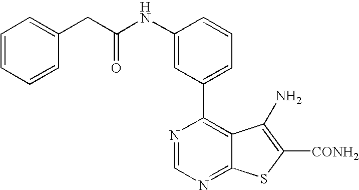 |
| 292 | 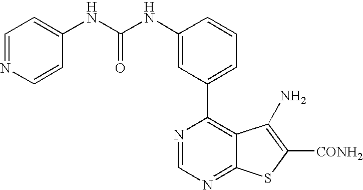 |
| 293 | 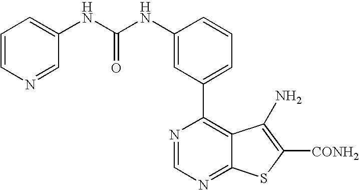 |
| 294 | 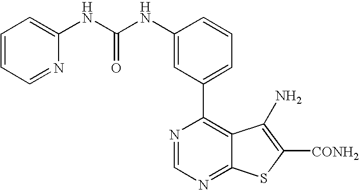 |
| 295 | 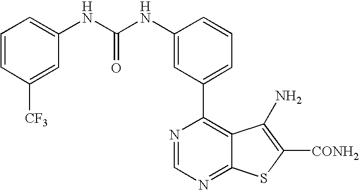 |
| 296 | 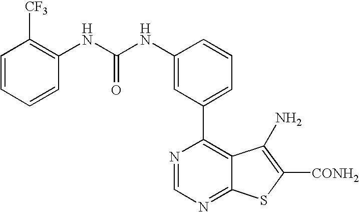 |

4.2 Methods for Making the Compounds

Illustrative Compounds can be made using conventional organic syntheses and using commercially available starting materials. By way of example and not limitation, Compounds having the formula I can be prepared as outlined in Schemes 1-10, below.

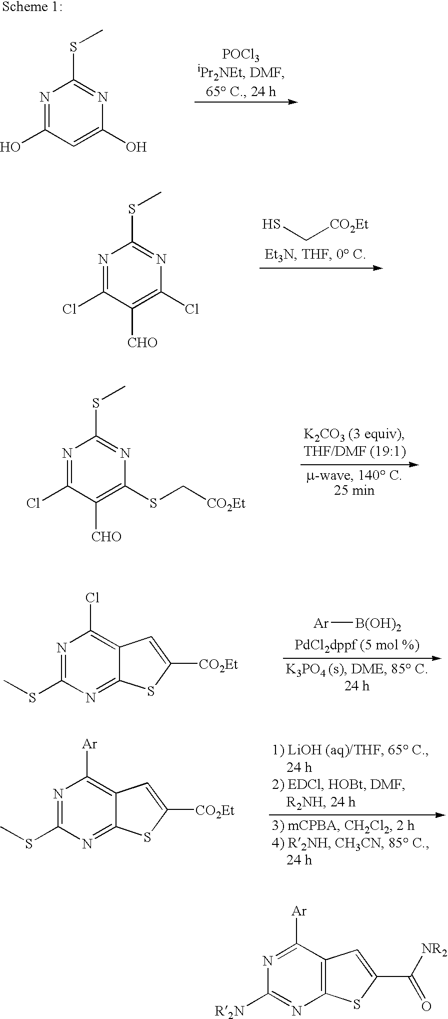

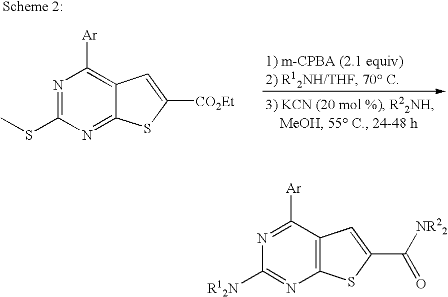

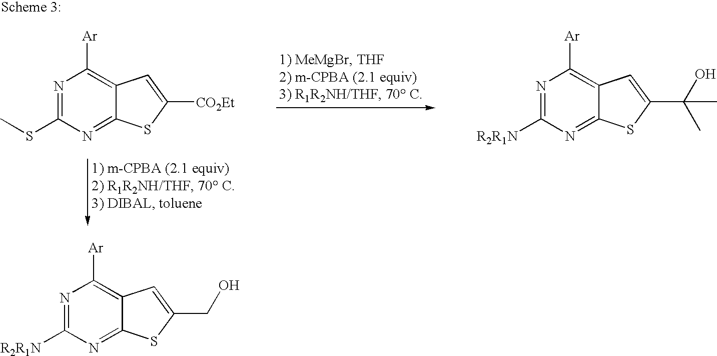

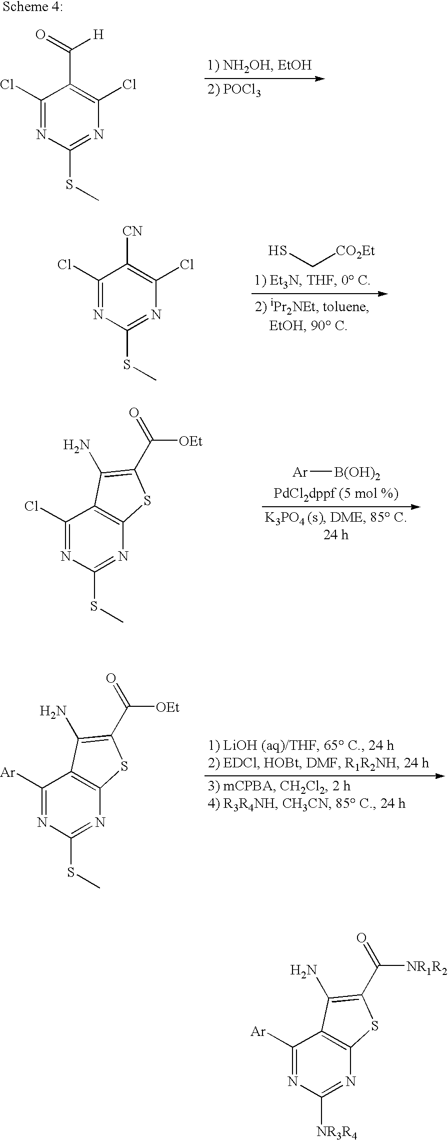

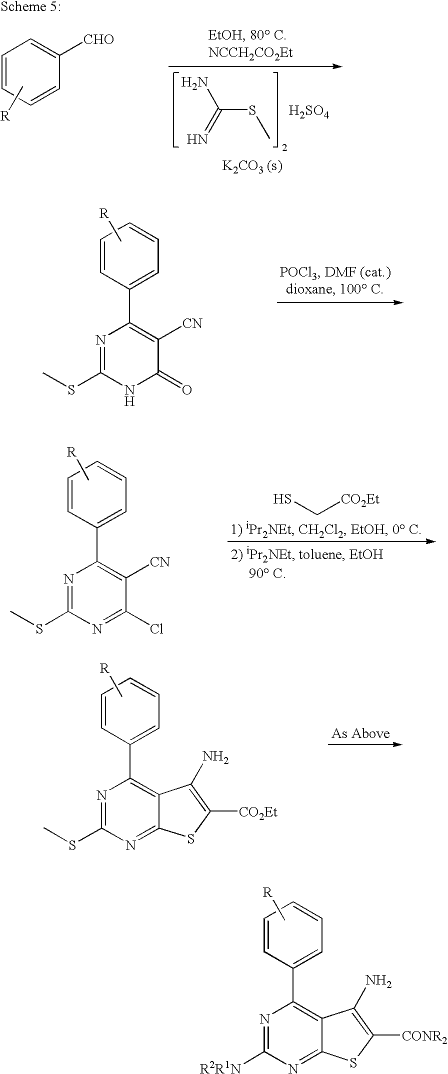

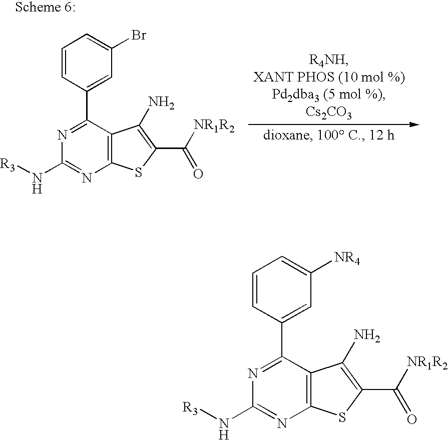

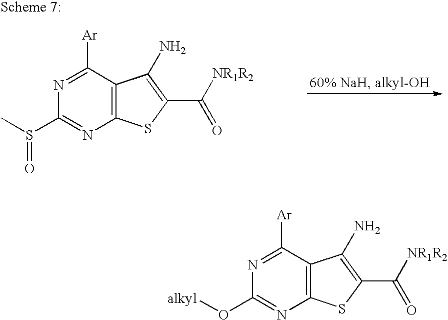

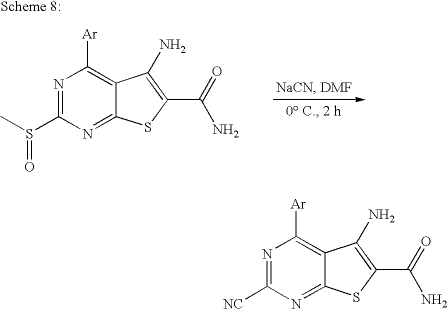

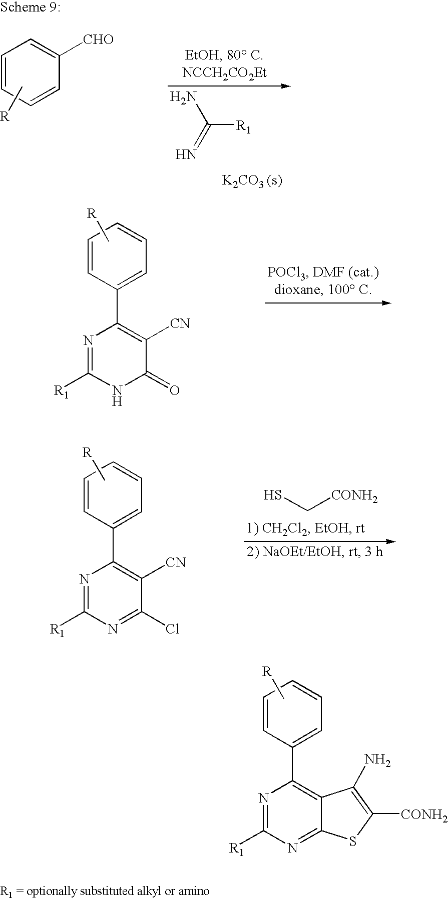

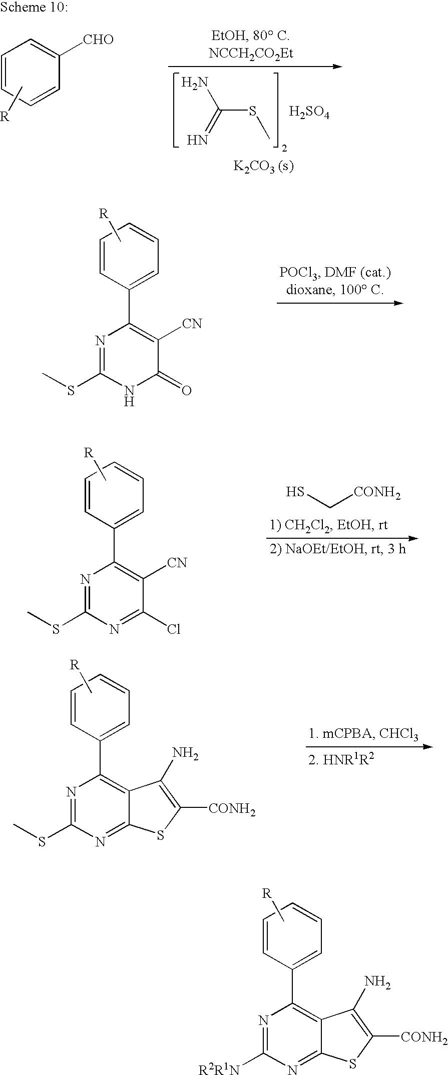

Once synthesized, a Compound can be isolated from chemical precursors or other chemicals using standard purification techniques such as, for example, chromatography (e.g., flash column chromatography and HPLC), asymmetric methods of synthesis, recrystallization and differential solubility.

4.3 Uses of the Compounds

Provided herein are therapies which involve administering an effective amount of one or more Compounds, or a composition thereof, to a patient (e.g., a patient in need thereof), such as a human patient, for treating, preventing, managing, and/or ameliorating a disease or disorder described herein, or one or more symptoms thereof, such as a disease or disorder associated with TNFα and/or PDE4 and/or B-RAF (e.g., a disease or disorder associated with aberrant TNFα and/or PDE4 and/or B-RAF activity).

In one embodiment, provided herein are methods for treating, preventing or managing cancer, inflammatory disorders, cognition and memory disorders or autoimmune disorders by administering to a patient (e.g., a patient in need thereof) an effective amount of one or more Compounds.

Also provided herein are methods for treating, preventing, managing, or ameliorating a disease or disorder described herein, or one or more symptoms thereof, such as a disease or disorder associated with TNFα and/or PDE4 and/or B-RAF, said methods comprising administering to a patient (e.g., a patient in need thereof) an effective amount of one or more Compounds and one or more other therapies (e.g., one or more prophylactic or therapeutic agents) that are currently being used, have been used, or are known to be useful in the treatment, prevention, management or amelioration of a disease or disorder associated with TNFα and/or PDE4 and/or B-RAF. The prophylactic or therapeutic agents of the combination therapies provided herein can be administered sequentially or concurrently.

The prophylactic or therapeutic agents of the combination therapies can be administered to a patient, in one embodiment a human patient, in the same pharmaceutical composition. In alternative embodiments, the prophylactic or therapeutic agents of the combination therapies can be administered concurrently to a patient in separate pharmaceutical compositions. The prophylactic or therapeutic agents can be administered to a patient by the same or different routes of administration.

In one embodiment, provided herein are methods for treating, preventing, managing and/or ameliorating septic shock, sepsis, endotoxic shock, endotoxemia, hemodynamic shock and sepsis syndrome, post ischemic reperfusion injury, chronic pulmonary inflammatory disease, COPD, chronic bronchitis, cosinophilic granuloma, myocardial infarction, stroke, circulatory shock, malaria, mycobacterial infection, meningitis, psoriasis, congestive heart failure, fibrotic disease, cachexia, graft rejection, graft versus host disease, cancer, auto-immune disease, viral infections (including, but not limited to, viral infections associated with the cytomegalia virus, influenza virus, or herpes virus), opportunistic infections in AIDS, hypercalcemia, rheumatoid arthritis, rheumatoid spondylitis, osteoarthritis and other arthritic conditions or bone resorption diseases, Crohn's disease, ulcerative colitis, multiple sclerosis, systemic lupus erythrematosis, allergic rhinoconjunctivitis, ENL in leprosy, radiation damage, asthma, atopic dermatitis, autoimmune diabetes, diabetes insipidus, autoimmune encephalomyelitis, adult respiratory system, silica- or asbestos-induced lung fibrosis, pulmonary sarcoidosis, depression, dementia (including, but not limited to, multi-infarct dementia, and hyperoxic alveolar injury, stroke related memory loss, Alzheimer's disease, mild cognitive impairment, age-related cognitive decline, age-associated memory impairment, pre-senile dementia, vascular (stroke-related) dementia, Lewy body disease, fronto-temporal lobar degeneration, Pick's disease, primary progressive aphasia, corticobasal degeneration, traumatic brain injury, dementia associated with Parkinsonism, memory impairment secondary to sleep disorders, and dementia in mentally retarded adults, or a symptom thereof, comprising administering to a patient (e.g., a patient in need thereof) an effective amount of one of more Compounds.

In a particular embodiment, provided herein are methods for treating, preventing, managing or ameliorating one or more cancers of the head, neck, eye, skin, mouth, throat, esophagus, chest, bone, lung, colon, sigmoid, rectum, stomach, prostate, breast, ovary, testicle, kidney, liver, pancreas, brain, intestine, heart or adrenals, or a symptom thereof, comprising administering to a patient (e.g., a patient in need thereof) an effective amount of one of more Compounds. In specific embodiments, cancer, malignancy or dysproliferative changes (such as metaplasias and dysplasias), or hyperproliferative disorders, are treatable or preventable in the ovary, breast, colon, lung, skin, pancreas, prostate, bladder, kidney or uterus. In other specific embodiments, sarcoma, melanoma, or leukemia is treatable or preventable.

In one embodiment, the Compounds are useful for treating or preventing cancers including prostate (such as hormone-insensitive), Neuroblastoma, Lymphoma (such as follicular or Diffuse Large B-cell), Breast (such as Estrogen-receptor positive), Colorectal, Endometrial, Ovarian, Lymphoma (such as non-Hodgkin's), Lung (such as Small cell), Testicular (such as germ cell), Thyroid (such as papillary thyroid carcinoma and anaplastic thyroid carcinoma) and Melanoma.

More particularly, cancers and related disorders that can be treated or prevented by methods and compositions provided herein include but are not limited to the following: Leukemias such as but not limited to, acute leukemia, acute lymphocytic leukemia, acute myelocytic leukemias such as myeloblastic, promyelocytic, myelomonocytic, monocytic, erythroleukemia leukemias and myelodysplastic syndrome (or a symptom thereof such as anemia, thrombocytopenia, neutropenia, bicytopenia or pancytopenia), refractory anemia (RA), RA with ringed sideroblasts (RARS), RA with excess blasts (RAEB), RAEB in transformation (RAEB-T), preleukemia and chronic myelomonocytic leukemia (CMML), chronic leukemias such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenström's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, metastatic cancers, neurilemmoma, rhabdomyosarcoma, synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer, including, but not limited to, adenocarcinoma, lobular (small cell) carcinoma, intraductal carcinoma, medullary breast cancer, mucinous breast cancer, tubular breast cancer, papillary breast cancer, primary cancers, Paget's disease, and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; esophageal cancers such as but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma, gallbladder cancers such as adenocarcinoma; cholangiocarcinomas such as but not limited to pappillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell cancer, adenocarcinoma, hypemephroma, fibrosarcoma, transitional cell cancer (renal pelvis and/or uterer); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangio-endotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J.B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

Accordingly, the methods and compositions provided herein are also useful in the treatment or prevention of a variety of cancers or other abnormal proliferative diseases, including (but not limited to) the following: carcinoma, including that of the bladder, breast, colon, kidney, liver, lung, ovary, pancreas, stomach, cervix, thyroid and skin; including squamous cell carcinoma; hematopoietic tumors of lymphoid lineage, including leukemia, acute lymphocytic leukemia, acute lymphoblastic leukemia, B-cell lymphoma, T-cell lymphoma, Berketts lymphoma; hematopoietic tumors of myeloid lineage, including acute and chronic myelogenous leukemias and promyelocytic leukemia; tumors of mesenchymal orignin, including fibrosarcoma and rhabdomyoscarcoma; other tumors, including melanoma, seminoma, tetratocarcinoma, neuroblastoma and glioma; tumors of the central and peripheral nervous system, including astrocytoma, glioblastoma multiforme, neuroblastoma, glioma, and schwannomas; solid and blood born tumors; tumors of mesenchymal origin, including fibrosafcoma, rhabdomyoscarama, and osteosarcoma; and other tumors, including melanoma, xenoderma pegmentosum, keratoactanthoma, seminoma, thyroid follicular cancer and teratocarcinoma. It is also contemplated that cancers caused by aberrations in apoptosis would also be treated by the methods and compositions disclosed herein. Such cancers may include but not be limited to follicular lymphomas, carcinomas with p53 mutations, hormone dependent tumors of the breast, prostate and ovary, and precancerous lesions such as familial adenomatous polyposis, and myelodysplastic syndromes. In specific embodiments, malignancy or dysproliferative changes (such as metaplasias and dysplasias), or hyperproliferative disorders, are treated or prevented in the ovary, bladder, breast, colon, lung, skin, pancreas, kidney or uterus. In other specific embodiments, sarcoma, melanoma, or leukemia is treated or prevented.

In a particular embodiment, the methods and compositions provided herein are also useful for treating, preventing or managing various types of lymphomas (i.e., a heterogenous group of neoplasms arising in the reticuloendothelial and lymphatic systems), such as Non-Hodgkin's lymphoma (NHL) (i.e., a malignant monoclonal proliferation of lymphoid cells in sites of the immune system, including lymph nodes, bone marrow, spleen, liver and gastrointestinal tract). NHLs that Compounds are useful for treating or preventing include, but are not limited to, mantle cell lymphoma, MCL, lymphocytic lymphoma of intermediate differentiation, intermediate lymphocytic lymphoma, ILL, diffuse poorly differentiated lymphocytic lymphoma, PDL, centrocytic lymphoma, diffuse small-cleaved cell lymphoma, DSCCL, follicular lymphoma, and any type of the mantle cell lymphomas that can be seen under the microscope (nodular, diffuse, blastic and mentle zone lymphoma).

In another embodiment, the methods and compositions provided herein are also useful for administration to patients in need of a bone marrow transplant to treat a malignant disease (e.g., patients suffering from acute lymphocytic leukemia, acute myelogenous leukemia, chronic myelogenous leukemia, chronic lymphocytic leukemia, myelodysplastic syndrome (“preleukemia”), monosomy 7 syndrome, non-Hodgkin's lymphoma, neuroblastoma, brain tumors, multiple myeloma, testicular germ cell tumors, breast cancer, lung cancer, ovarian cancer, melanoma, glioma, sarcoma or other solid tumors), those in need of a bone marrow transplant to treat a non-malignant disease (e.g., patients suffering from hematologic disorders, congenital immunodeficiencies, mucopolysaccharidoses, lipidoses, osteoporosis, Langerhan's cell histiocytosis, Lesch-Nyhan syndrome or glycogen storage diseases), those undergoing chemotherapy or radiation therapy, those preparing to undergo chemotherapy or radiation therapy and those who have previously undergone chemotherapy or radiation therapy.

In another embodiment, provided herein are methods for the treatment of myeloproliferative disorders or myelodysplastic syndromes, comprising administering to a patient in need thereof an effective amount of a Compound or a composition thereof. In certain embodiments, the myeloproliferative disorder is polycythemia rubra vera; primary thrombocythemia; chronic myelogenous leukemia; acute or chronic granulocytic leukemia; acute or chronic myelomonocytic leukemia; myelofibro-erythroleukemia; or agnogenic myeloid metaplasia.

In another embodiment, provided herein are methods for the treatment of cancer or tumors resistant to other kinase inhibitors such as imatinib mesylate (STI-571 or Gleevec™) treatment, comprising administering to a patient in need thereof an effective amount of a Compound or a composition thereof. In a particular embodiment, provided herein are methods for the treatment of leukemias, including, but not limited to, gastrointestinal stromal tumor (GIST), acute lymphocytic leukemia or chronic myelocytic leukemia resistant to imatinib mesylate (STI-571 or Gleevec™) treatment, comprising administering to a patient in need thereof an effective amount of a Compound or a composition thereof.

In a specific embodiment, provided herein are methods for treating or preventing leukemia (i.e., malignant neoplasms of the blood-forming tissues) including, but not limited to, chronic lymphocytic leukemia, chronic myelocytic leukemia, acute lymphoblastic leukemia, acute myelogenous leukemia and acute myeloblastic leukemia. The leukemia can be relapsed, refractory or resistant to conventional therapy. The term “relapsed” refers to a situation where patients who have had a remission of leukemia after therapy have a return of leukemia cells in the marrow and a decrease in normal blood cells. The term “refractory or resistant” refers to a circumstance where patients, even after intensive treatment, have residual leukemia cells in their marrow.

The various types of the cancers are described in U.S. provisional application No. 60/380,842, filed May 17, 2002, the entireties of which are incorporated herein by reference (see, e.g., Section 2.2. Types of Cancers). Specific cancers include, but are not limited to, leukemias such as chronic lymphocytic leukemia, chronic myelocytic leukemia, acute lymphoblastic leukemia, acute myelogenous leukemia, and acute myeloblastic leukemia; advanced malignancy, amyloidosis, neuroblastoma, meningioma, hemangiopericytoma, multiple brain metastase, glioblastoma multiforms, glioblastoma, brain stem glioma, poor prognosis malignant brain tumor, malignant glioma, recurrent malignant giolma, anaplastic astrocytoma, anaplastic oligodendroglioma, neuroendocrine tumor, rectal adenocarcinoma, Dukes C & D colorectal cancer, unresectable colorectal carcinoma, metastatic hepatocellular carcinoma, Kaposi's sarcoma, karotype acute myeloblastic leukemia, Hodgkin's lymphoma, non-Hodgkin's lymphoma, cutaneous T-Cell lymphoma, cutaneous B-Cell lymphoma, diffuse large B-Cell lymphoma, low grade follicular lymphoma, malignant melanoma, malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, leiomyosarcoma, fibrodysplasia ossificans progressive, hormone refractory prostate cancer, resected high-risk soft tissue sarcoma, unrescectable hepatocellular carcinoma, Waldenstrom's macroglobulinemia, smoldering myeloma, indolent myeloma, fallopian tube cancer, androgen independent prostate cancer, androgen dependent stage 1V non-metastatic prostate cancer, hormone-insensitive prostate cancer, chemotherapy-insensitive prostate cancer, papillary thyroid carcinoma, follicular thyroid carcinoma, medullary thyroid carcinoma, and leiomyoma. In one embodiment, the cancer is primary or metastatic. In another embodiment, the cancer is relapsed, refractory or resistance to chemotherapy or radiation; in particular, refractory to thalidomide.

Further provide herein are methods for treating patients who have been previously treated for cancer, but are non-responsive to standard therapies, as well as those who have not previously been treated. Also provided herein are methods for treating patients regardless of patient's age, although some cancers are more common in certain age groups. Still further provided herein are methods for treating patients who have undergone surgery in an attempt to treat the cancer at issue, as well as those who have not. Because patients with cancer have heterogenous clinical manifestations and varying clinical outcomes, the treatment given to a patient may vary, depending on his/her prognosis. The skilled clinician will be able to readily determine without undue experimentation specific secondary agents, types of surgery, and types of non-drug based standard therapy that can be effectively used to treat an individual patient with cancer.

In another embodiment, provided herein are methods of inhibiting the growth of a cell, said cell being derived from a cancer or neoplasm, comprising contacting a cell with an effective amount of one or more Compounds or a composition thereof.

In another embodiment, provided herein are methods of inhibiting TNFα in a cell comprising contacting a cell expressing TNFα with an effective amount of one or more Compounds or a composition thereof.

In another embodiment, provided herein are methods of inhibiting PDE4 in a cell comprising contacting a cell expressing PDE4 with an effective amount of one or more Compounds or a composition thereof.

In another embodiment, provided herein are methods of inhibiting B-RAF in a cell comprising contacting a cell expressing B-RAF with an effective amount of one or more Compounds or a composition thereof.

Further provided herein are methods of reducing, treating and/or preventing adverse or undesired effects associated with conventional therapy including, but not limited to, surgery, chemotherapy, radiation therapy, hormonal therapy, biological therapy and immunotherapy. Compounds and second active agents can be administered to a patient prior to, during, or after the occurrence of the adverse effect associated with conventional therapy.

4.4 Compositions and Methods of Administration

Provided herein are compositions for the treatment, prevention, management or amelioration of a disease or disorder, or a symptom thereof, characterized by or associated with TNFα and/or PDE4 and/or B-RAF activity (e.g., a disease or disorder associated with aberrant TNFα and/or PDE4 and/or B-RAF activity). In one embodiment, a composition comprises an effective amount of one or more Compounds. In another embodiment, a composition comprises an effective amount of one or more Compounds and an effective amount of one or more other agents useful for the treatment, prevention, management or amelioration of a disease or disorder characterized by or associated with TNFα and/or PDE4 and/or B-RAF activity (e.g., a disease or disorder associated with aberrant TNFα and/or PDE4 and/or B-RAF activity).

In one embodiment, a composition provided herein consists of an effective amount of one or more Compounds.

In another embodiment, a composition provided herein is a pharmaceutical composition or a single unit dosage form. Pharmaceutical compositions and single unit dosage forms provided herein can comprise an effective amount of a Compound and one or more pharmaceutically acceptable carriers. In a specific embodiment and in this context, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete)), excipient, or vehicle with which a composition provided herein is administered. Such pharmaceutically acceptable carriers can be sterile liquids, such as water and oils, including those of petroleum, animal, vegetable or synthetic origin, such as peanut oil, soybean oil, mineral oil, sesame oil and the like. Water can be a carrier when the pharmaceutical composition is administered intravenously. Saline solutions and aqueous dextrose and glycerol solutions can also be employed as liquid carriers, particularly for injectable solutions. Examples of suitable pharmaceutical carriers are described in “Remington's Pharmaceutical Sciences” by E. W. Martin.

Typical pharmaceutical compositions and dosage forms comprise one or more excipients. Suitable excipients are well-known to those skilled in the art of pharmacy, and non-limiting examples of suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Whether a particular excipient is suitable for incorporation into a pharmaceutical composition or dosage form depends on a variety of factors well known in the art including, but not limited to, the way in which the dosage form will be administered to a patient and the specific active ingredients in the dosage form. The composition or single unit dosage form, if desired, can also contain minor amounts of wetting or emulsifying agents, or pH buffering agents.

Lactose-free compositions can comprise excipients that are well known in the art and are listed, for example, in the U.S. Pharmocopeia (USP) SP (XXI)/NF (XVI). In general, lactose-free compositions comprise an active ingredient, a binder/filler, and a lubricant in pharmaceutically compatible and pharmaceutically acceptable amounts. Illustrative lactose-free dosage forms comprise an active ingredient, microcrystalline cellulose, pre-gelatinized starch, and magnesium stearate.

Further provided herein are anhydrous pharmaceutical compositions and dosage forms comprising a Compound, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine can be anhydrous if substantial contact with moisture and/or humidity during manufacturing, packaging, and/or storage is expected.

An anhydrous pharmaceutical composition can be prepared and stored such that its anhydrous nature is maintained. Accordingly, anhydrous compositions can be packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Further provided herein are pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

The pharmaceutical compositions and single unit dosage forms can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Such compositions and dosage forms will contain a prophylactically or therapeutically effective amount of a prophylactic or therapeutic agent, in one embodiment in purified form, together with a suitable amount of carrier so as to provide the form for proper administration to the patient. The formulation should suit the mode of administration. In one embodiment, the pharmaceutical compositions or single unit dosage forms are sterile and in suitable form for administration to a patient (e.g., a patient in need thereof), in one embodiment an animal patient, in another embodiment a mammalian patient, and in a further embodiment a human patient.

A pharmaceutical composition provided herein can be formulated to be compatible with its intended route of administration. Examples of routes of administration include, but are not limited to, parenteral, e.g., intravenous, intradermal, subcutaneous, oral (e.g., inhalation), intranasal, transdermal (topical), transmucosal, intra-tumoral, intra-synovial and rectal administration. In a specific embodiment, the composition is formulated in accordance with routine procedures as a pharmaceutical composition adapted for intravenous, subcutaneous, intramuscular, oral, intranasal or topical administration to human beings. In one embodiment, a pharmaceutical composition is formulated in accordance with routine procedures for subcutaneous administration to human beings. Typically, compositions for intravenous administration are solutions in sterile isotonic aqueous buffer. Where necessary, the composition may also include a solubilizing agent and a local anesthetic such as lignocane to ease pain at the site of the injection. Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms provided herein will typically vary depending on their use. For example, a dosage form used in the acute treatment of inflammation or a related disorder may contain larger amounts of one or more of the active ingredients it comprises than a dosage form used in the chronic treatment of the same disease. Also, the therapeutically effective dosage form may vary among different types of cancer. Similarly, a parenteral dosage form may contain smaller amounts of one or more of the active ingredients it comprises than an oral dosage form used to treat the same disease or disorder. These and other ways in which specific dosage forms provided herein will vary from one another will be readily apparent to those skilled in the art. See, e.g., Remington's Pharmaceutical Sciences, 18th ed., Mack Publishing, Easton Pa. (1990).

Generally, the ingredients of compositions provided herein are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration. Typical dosage forms provided herein comprise a Compound, or a pharmaceutically acceptable salt, solvate or hydrate thereof lie within the range of from about 1 mg to about 1000 mg per day, given as a single once-a-day dose in the morning but can be divided doses throughout the day taken with food.

In one embodiment, a Compound is administered alone (i.e., without a pharmaceutically acceptable carrier) to a patient (e.g., a patient in need thereof). In a particular embodiment, a Compound is administered alone as a solid or as a solid in a capsule without a pharmaceutically acceptable carrier.

4.4.1 Oral Dosage Forms

Pharmaceutical compositions provided herein that are suitable for oral administration can be presented as discrete dosage forms including, but not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990).

Typical oral dosage forms provided herein are prepared by combining a Compound in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms provided herein include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Examples of fillers suitable for use in the pharmaceutical compositions and dosage forms provided herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in certain pharmaceutical compositions provided herein is typically present in from about 50 to about 99 weight percent of the pharmaceutical composition or dosage form.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. A specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Disintegrants are used in the compositions provided herein to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms provided herein. The amount of disintegrant used varies based upon the type of formulation, and is readily discernible to those of ordinary skill in the art. Typical pharmaceutical compositions comprise from about 0.5 to about 15 weight percent of disintegrant, specifically from about 1 to about 5 weight percent of disintegrant.

Disintegrants that can be used in pharmaceutical compositions and dosage forms provided herein include, but are not limited to, agar-agar, alginic acid, calcium carbonate, microcrystalline cellulose, croscarmellose sodium, crospovidone, polacrilin potassium, sodium starch glycolate, potato or tapioca starch, pre-gelatinized starch, other starches, clays, other algins, other celluloses, gums, and mixtures thereof.

Lubricants that can be used in pharmaceutical compositions and dosage forms provided herein include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

4.4.2 Sustained or Controlled Release Dosage Forms

Compounds can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with a Compound. Accordingly, provided herein are single unit dosage forms suitable for oral administration such as, but not limited to, tablets, capsules, gelcaps, and caplets that are adapted for controlled-release.

Certain controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

4.4.3 Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms can be sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms are well known to those skilled in the art. Examples include, but are not limited to: Water for Injection USP; aqueous vehicles including, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the Compounds can also be incorporated into the parenteral dosage forms provided herein.

4.4.4 Transdermal, Topical & Mucosal Dosage Forms

Transdermal, topical, and mucosal dosage forms provided herein include, but are not limited to, ophthalmic solutions, sprays, aerosols, creams, lotions, ointments, gels, solutions, emulsions, suspensions, or other forms known to one of skill in the art. See, e.g., Remington's Pharmaceutical Sciences, 16th and 18th eds., Mack Publishing, Easton Pa. (1980 & 1990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels. Further, transdermal dosage forms include “reservoir type” or “matrix type” patches, which can be applied to the skin and worn for a specific period of time to permit the penetration of a desired amount of a Compound.

Suitable excipients (e.g., carriers and diluents) and other materials that can be used to provide transdermal, topical, and mucosal dosage forms provided herein are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof to form lotions, tinctures, creams, emulsions, gels or ointments, which are non-toxic and pharmaceutically acceptable. Moisturizers or humectants can also be added to pharmaceutical compositions and dosage forms if desired. Examples of such additional ingredients are well known in the art. See, e.g., Remington's Pharmaceutical Sciences, 16th and 18th eds., Mack Publishing, Easton Pa. (1980 & 1990).

Depending on the specific tissue to be treated, additional components can be used prior to, in conjunction with, or subsequent to treatment with a Compound. For example, penetration enhancers can be used to assist in delivering a Compound to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 (polysorbate 80) and Span 60 (sorbitan monostearate).

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

4.4.5 Dosage and Frequency of Administration

The amount of a Compound, or composition thereof, which will be effective in the prevention, treatment, management, or amelioration of a disorder (e.g., a disorder characterized by or associated with aberrant PDE4 and/or TNFα and/or B-RAF activity), or one or more symptoms thereof, will vary with the nature and severity of the disease or condition, and the route by which the Compound or composition thereof is administered. The frequency and dosage will also vary according to factors specific for each patient depending on the specific therapy (e.g., therapeutic or prophylactic agents) administered, as well as age, body, weight, response, and the past medical history of the patient. Effective doses may be extrapolated from dose-response curves derived from in vitro or animal model test systems. Suitable regiments can be selected by one skilled in the art by considering such factors and by following, for example, dosages reported in the literature and recommended in the Physician's Desk Reference (59^{th} ed., 2005).

Exemplary doses of a Compound include milligram or microgram amounts of the Compound per kilogram of patient or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram).

In general, the recommended daily dose range of a Compound for the conditions described herein lie within the range of from about 0.01 mg to about 5000 mg per day, given as a single once-a-day dose which can be administered as divided doses throughout a day. In one embodiment, the daily dose is administered twice daily in equally divided doses. Specifically, a daily dose range should be from about 5 mg to about 1000 mg per day, more specifically, between about 10 mg and about 500 mg per day, and more specifically, between about 25 mg and about 250 mg per day. In managing the patient, the therapy can be initiated at a lower dose, such as about 1 mg to about 25 mg, and increased if necessary up to about 200 mg to about 1000 mg or 5000 mg per day as either a single dose or divided doses, depending on the patient's global response. It may be necessary to use dosages of the Compound outside the ranges disclosed herein in some cases, as will be apparent to those of ordinary skill in the art. Furthermore, it is noted that the clinician or treating physician will know how and when to interrupt, adjust, or terminate therapy in conjunction with individual patient response.

Different therapeutically effective amounts may be applicable for different diseases and conditions, as will be readily known by those skilled in the art. Further, when a patient is administered multiple dosages of a Compound, not all of the dosages need be the same. For example, the dosage administered to the patient can be increased to improve the prophylactic or therapeutic effect of the Compound or it may be decreased to reduce one or more side effects that a particular patient is experiencing.

In a specific embodiment, the dosage of a Compound administered to prevent, treat, manage, or ameliorate a disorder (e.g., a disorder characterized by or associated with aberrant TNFα or PDE4 activity), or one or more symptoms thereof, in a patient is 150 μg/kg, 250 μg/kg, 500 μg/kg, 1 mg/kg, 5 mg/kg, 10 mg/kg, 25 mg/kg, 50 mg/kg, 75 mg/kg, 100 mg/kg, 125 mg/kg, 150 mg/kg, or 200 mg/kg or more of a patient's body weight. In another embodiment, the dosage of a composition provided herein or a Compound administered to prevent, treat, manage, or ameliorate a disorder (e.g., a disorder characterized by or associated with aberrant TNFα and/or PDE4 and/or B-RAF activity), or one or more symptoms thereof, in a patient (e.g., a patient in need thereof) is a unit dose of 0.1 mg to 20 mg, 0.1 mg to 15 mg, 0.1 mg to 12 mg, 0.1 mg to 10 mg, 0.1 mg to 8 mg, 0.1 mg to 7 mg, 0.1 mg to 5 mg, 0.1 mg to 2.5 mg, 0.25 mg to 20 mg, 0.25 mg to 15 mg, 0.25 mg to 12 mg, 0.25 mg to 10 mg, 0.25 mg to 8 mg, 0.25 mg to 7 mg, 0.25 mg to 5 mg, 0.5 mg to 2.5 mg, 1 mg to 20 mg, 1 mg to 15 mg, 1 mg to 12 mg, 1 mg to 10 mg, 1 mg to 8 mg, 1 mg to 7 mg, 1 mg to 5 mg, or 1 mg to 2.5 mg. The dosages of prophylactic or therapeutic agents other than Compounds, which have been or are currently being used to prevent, treat, manage, or ameliorate a disorder (e.g., a disorder characterized by or associated with aberrant TNFα and/or PDE4 and/or B-RAF activity), or one or more symptoms thereof, can be used in the combination therapies provided herein. In one embodiment, dosages lower than those which have been or are currently being used to prevent, treat, manage, or ameliorate a disorder (e.g., a disorder characterized by or associated with aberrant TNFα and/or PDE4 and/or B-RAF activity), or one or more symptoms thereof, are used in the combination therapies provided herein. The recommended dosages of agents currently used for the prevention, treatment, management, or amelioration of a disorder (e.g., a disorder characterized by or associated with aberrant TNFα and/or PDE4 and/or B-RAF activity), or one or more symptoms thereof, can obtained from any reference in the art including, but not limited to, Hardman et al., eds., 1996, Goodman & Gilman's The Pharmacological Basis Of Basis Of Therapeutics 9^{th} Ed, Mc-Graw-Hill, New York; Physician's Desk Reference (PDR) 59^{th} ed., 2005, Medical Economics Co., Inc., Montvale, N.J., which are incorporated herein by reference in its entirety.

In certain embodiments, one or more Compounds and one or more other the therapies (e.g., prophylactic or therapeutic agents) are cyclically administered. Cycling therapy involves the administration of a first therapy (e.g., a first prophylactic or therapeutic agents) for a period of time, followed by the administration of a second therapy (e.g., a second prophylactic or therapeutic agents) for a period of time, followed by the administration of a third therapy (e.g., a third prophylactic or therapeutic agents) for a period of time and so forth, and repeating this sequential administration, i.e., the cycle in order to reduce the development of resistance to one of the agents, to avoid or reduce the side effects of one of the agents, and/or to improve the efficacy of the treatment.

In certain embodiments, administration of the same Compound can be repeated and the administrations can be separated by at least 1 day, 2 days, 3 days, 5 days, 10 days, 15 days, 30 days, 45 days, 2 months, 75 days, 3 months, or 6 months. In other embodiments, administration of the same prophylactic or therapeutic agent can be repeated and the administration can be separated by at least at least 1 day, 2 days, 3 days, 5 days, 10 days, 15 days, 30 days, 45 days, 2 months, 75 days, 3 months, or 6 months.

In a specific embodiment, provided herein are methods for preventing, treating, managing, or ameliorating a disorder (e.g., a disorder characterized by or associated with aberrant TNFα and/or PDE4 and/or B-RAF activity), or one or more symptoms thereof, said methods comprising administering to a patient in need thereof a dose of at least 150 μg/kg, in one embodiment at least 250 μg/kg, at least 500 μg/kg, at least 1 mg/kg, at least 5 mg/kg, at least 10 mg/kg, at least 25 mg/kg, at least 50 mg/kg, at least 75 mg/kg, at least 100 mg/kg, at least 125 mg/kg, at least 150 mg/kg, or at least 200 mg/kg or more of one or more Compounds once every 3 days, once every 4 days, once every 5 days, once every 6 days, once every 7 days, once every 8 days, once every 10 days, once every two weeks, once every three weeks, or once a month.

In a specific embodiment, provided herein are methods for preventing, treating, managing, or ameliorating a disorder (e.g., a disorder characterized by or associated with aberrant TNFα and/or PDE4 and/or B-RAF activity), or one or more symptoms thereof, said method comprising: (a) administering to a patient (e.g., a patient in need thereof) a dose of at least 150 μg/kg, in one embodiment at least 250 μg/kg, at least 500 μg/kg, at least 1 mg/kg, at least 5 mg/kg, at least 10 mg/kg, at least 25 mg/kg, at least 50 mg/kg, at least 75 mg/kg, at least 100 mg/kg, at least 125 mg/kg, at least 150 mg/kg, or at least 200 mg/kg or more of one or more Compounds; and (b) administering one or more subsequent doses to said patient when the mean absolute lymphocyte count in said patient is at least approximately 500 cells/mm³, in one embodiment at least approximately 600 cells/mm³, at least approximately 700 cells/mm³, at least approximately 750 cells/mm³, at least approximately 800 cells/mm³, at least approximately 850 cells/mm³, or at least approximately 900 cells/mm³.

4.5 Agents Useful in Combination with the Compounds

Provided herein are methods for preventing, managing, treating, or ameliorating disorders, or a symptom thereof, associated with TNFα and/or PDE4 and/or B-RAF comprising administering to a patient (e.g., a patient in need thereof) an effective amount of one or more Compounds in combination with one or more other TNFα and/or PDE4 and/or B-RAF therapies (e.g., one or more prophylactic or therapeutic agents).

Any TNFα antagonist well-known to one of skill in the art can be used in the compositions and methods provided herein. Non-limiting examples of TNFα antagonists include proteins, polypeptides, peptides, fusion proteins, antibodies (e.g., human, humanized, chimeric, monoclonal, polyclonal, Fvs, ScFvs, Fab fragments, F(ab)₂ fragments, and antigen-binding fragments thereof) such as antibodies that immunospecifically bind to TNFα, nucleic acid molecules (e.g., antisense molecules or triple helices), organic molecules, inorganic molecules, and small molecules that block, reduce, inhibit or neutralize a function, an activity and/or the expression of TNFα. In various embodiments, a TNFα antagonist reduces the function, activity and/or expression of TNFα by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% relative to a control such as phosphate buffered saline (PBS).

Examples of antibodies that immunospecifically bind to TNFα include, but are not limited to, infliximab (REMICADE®; Centacor), D2E7 (Abbott Laboratories/Knoll Pharmaceuticals Co., Mt. Olive, N.J.), CDP571 which is also known as HUMICADE™ and CDP-870 (both of Celltech/Pharmacia, Slough, U.K.), and TN3-19.12 (Williams et al., 1994, Proc. Natl. Acad. Sci. USA 91: 2762-2766; Thorbecke et al., 1992, Proc. Natl. Acad. Sci. USA 89:7375-7379). Further provided in the compositions and methods described herein are uses of antibodies that immunospecifically bind to TNFα, including those disclosed in the following U.S. patents: U.S. Pat. Nos. 5,136,021; 5,147,638; 5,223,395; 5,231,024; 5,334,380; 5,360,716; 5,426,181; 5,436,154; 5,610,279; 5,644,034; 5,656,272; 5,658,746; 5,698,195; 5,736,138; 5,741,488; 5,808,029; 5,919,452; 5,958,412; 5,959,087; 5,968,741; 5,994,510; 6,036,978; 6,114,517; and 6,171,787; each of which are herein incorporated by reference in their entirety. Examples of soluble TNFα receptors include, but are not limited to, sTNF-R1 (Amgen), etanercept (ENBREL™; Immunex) and its rat homolog RENBREL™, soluble inhibitors of TNFα derived from TNFrI, TNFrII (Kohno et al., 1990, Proc. Natl. Acad. Sci. USA 87:8331-8335), and TNFα Inh (Seckinger et al., 1990, Proc. Natl. Acad. Sci. USA 87:5188-5192).

In one embodiment, a TNFα antagonist used in the compositions and methods provided herein is a soluble TNFα receptor. In a specific embodiment, a TNFα antagonist used in the compositions and methods provided herein is etanercept (ENBREL™; Immunex) or a fragment, derivative or analog thereof. In another embodiment, a TNFα antagonist used in the compositions and methods provided herein is an antibody that immunospecifically binds to TNFα. In a specific embodiment, a TNFα antagonist used in the compositions and methods provided herein is infliximab (REMICADE®; Centacor) a derivative, analog or antigen-binding fragment thereof.

TNFα antagonists and their dosages, routes of administration and recommended usage are known in the art and have been described in such literature as the Physician's Desk Reference (59^{th} ed., 2005).

Any PDE4 inhibitor well-known to one of skill in the art can be used in the compositions and methods provided herein. Examples of PDE4 inhibitors that can be used in combination with a Compound include, but are not limited to, Cilomilast, Roflumilast, BAY 19-8004 (see Surton and Fitzgerald, Chest. 121(5 Suppl):192S-196S (2002)) and AWD 12-281 (see Gutke et al., Curr. Opin. Investig. Drugs 6(11): 1149-1158 (2005)).

Any B-RAF inhibitor well-known to one of skill in the art can be used in the compositions and methods provided herein. Examples of B-RAF inhibitors that can be used in combination with a Compound include, but are not limited to, sorafenib, PLX3331 and PLX4032.

In one embodiment, provided herein are compositions (e.g., pharmaceutical compositions) comprising an effective amount of one or more Compounds and an effective amount of Tykerb (lapatinib), further optionally comprising a carrier, diluent or excipient.

In another embodiment, provided herein are methods for treating cancer, in one embodiment brain cancer, in another embodiment breast cancer that has metastasized to the brain, comprising administering an effective amount of one or more Compounds in combination with an effective amount of Tykerb (lapatinib).

A Compound can also be combined with other pharmacologically active compounds (“second active agents”) in the methods and compositions described herein. It is believed that certain combinations may work synergistically in the treatment of particular types diseases or disorders, and conditions and symptoms associated with such diseases or disorders. A Compound can also work to alleviate adverse effects associated with certain second active agents, and vice versa.

One or more second active ingredients or agents can be used in the methods and compositions described herein. Second active agents can be large molecules (e.g., proteins) or small molecules (e.g., synthetic inorganic, organometallic, or organic molecules).

Examples of large molecule second active agents include, but are not limited to, hematopoietic growth factors, cytokines, and monoclonal and polyclonal antibodies. Specific examples of the active agents are anti-CD40 monoclonal antibodies (such as, for example, SGN-40); histone deacetylyase inhibitors (such as, for example, SAHA and LAQ 824); heat-shock protein-90 inhibitors (such as, for example, 17-AAG); insulin-like growth factor-1 receptor kinase inhibitors; vascular endothelial growth factor receptor kinase inhibitors (such as, for example, PTK787); insulin growth factor receptor inhibitors; lysophosphatidic acid acyltransrerase inhibitors; IkB kinase inhibitors; p38MAPK inhibitors; EGFR inhibitors (such as, for example, gefitinib and erlotinib HCL); HER-2 antibodies (such as, for example, trastuzumab (Herceptin®) and pertuzumab (Omnitarg™)); VEGFR antibodies (such as, for example, bevacizumab (Avastin™)); VEGFR inhibitors (such as, for example, flk-1 specific kinase inhibitors, SU5416 and ptk787/zk222584); P13K inhibitors (such as, for example, wortmannin); C-Met inhibitors (such as, for example, PHA-665752); monoclonal antibodies (such as, for example, rituximab (Rituxan®), tositumomab (Bexxar®), edrecolomab (Panorex®) and G250); and anti-TNF-α antibodies. Examples of small molecule active agents include, but are not limited to, small molecule anti-cancer agents and antibiotics (e.g., clarithromycin).

Specific second active compounds that can be combined with a Compound vary depending on the specific indication to be treated, prevented or managed.

For instance, for the treatment, prevention or management of cancer, second active agents include, but are not limited to: semaxanib; cyclosporin; etanercept; doxycycline; bortezomib; acivicin; aclarubicin; acodazole hydrochloride; acronine; adozelesin; aldesleukin; altretamine; ambomycin; ametantrone acetate; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; celecoxib; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; iproplatin; irinotecan; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; taxotere; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

Other second agents include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clathromycin; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; doxorubicin; droloxifene; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imatinib (Gleevec®), imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mitoguazone; mitolactol; mitomycin analogues; mitonafide; mitotoxin fibroblast growth factor-saporin; mitoxantrone; mofarotene; molgramostim; Erbitux, human chorionic gonadotrophin; monophosphoryl lipid A+myobacterium cell wall sk; mopidamol; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; nilutamide; nisamycin; nitric oxide modulators; nitroxide antioxidant; nitrullyn; oblimersen (Genasense®); O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stipiamide; stromelysin inhibitors; sulfinosine; superactive vasoactive intestinal peptide antagonist; suradista; suramin; swainsonine; tallimustine; tamoxifen methiodide; tauromustine; tazarotene; tecogalan sodium; tegafuir; tellurapyrylium; telomerase inhibitors; temoporfin; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; velaresol; veramine; verdins; verteporfin; vinorelbine; vinxaltine; vitaxin; vorozole; zanoterone; zeniplatin; zilascorb; and zinostatin stimalamer.

Specific second active agents include, but are not limited to, 2-methoxyestradiol, telomestatin, inducers of apoptosis in mutiple myeloma cells (such as, for example, TRAIL), bortezomib, statins, semaxanib, cyclosporin, etanercept, doxycycline, bortezomib, oblimersen (Genasense®), remicade, docetaxel, celecoxib, melphalan, dexamethasone (Decadron®), steroids, gemcitabine, cisplatinum, temozolomide, etoposide, cyclophosphamide, temodar, carboplatin, procarbazine, gliadel, tamoxifen, topotecan, methotrexate, Arisa®, taxol, taxotere, fluorouracil, leucovorin, irinotecan, xeloda, CPT-11, interferon alpha, pegylated interferon alpha (e.g., PEG INTRON-A), capecitabine, cisplatin, thiotepa, fludarabine, carboplatin, liposomal daunorubicin, cytarabine, doxetaxol, pacilitaxel, vinblastine, IL-2, GM-CSF, dacarbazine, vinorelbine, zoledronic acid, palmitronate, biaxin, busulphan, prednisone, bisphosphonate, arsenic trioxide, vincristine, doxorubicin (Doxil®), paclitaxel, ganciclovir, adriamycin, estramustine sodium phosphate (Emcyt®), sulindac, and etoposide.

Similarly, examples of specific second agents according to the indications to be treated, prevented, or managed can be found in the following references, all of which are incorporated herein in their entireties: U.S. Pat. Nos. 6,281,230 and 5,635,517; U.S. application Ser. Nos. 10/411,649, 10/483,213, 10/411,656, 10/693,794, 10/699,154, and 10/981,189; and U.S. provisional application Nos. 60/554,923, 60/565,172, 60/626,975, 60/630,599, 60/631,870, and 60/533,862.

Examples of additional second active agents include, but are not limited to, conventional therapeutics used to treat or prevent pain such as antidepressants, anticonvulsants, antihypertensives, anxiolytics, calcium channel blockers, muscle relaxants, non-narcotic analgesics, opioid analgesics, anti-inflammatories, cox-2 inhibitors, immunomodulatory agents, alpha-adrenergic receptor agonists or antagonists, immunosuppressive agents, corticosteroids, hyperbaric oxygen, ketamine, other anesthetic agents, NMDA antagonists, and other therapeutics found, for example, in the Physician's Desk Reference 2003. Specific examples include, but are not limited to, salicylic acid acetate (Aspirin®), celecoxib (Celebrex®), Enbrel®, ketamine, gabapentin (Neurontin®), phenyloin (Dilantin®), carbamazepine (Tegretol®), oxcarbazepine (Trileptal®), valproic acid (Depakene®), morphine sulfate, hydromorphone, prednisone, griseofulvin, penthonium, alendronate, dyphenhydramide, guanethidine, ketorolac (Acular®), thyrocalcitonin, dimethylsulfoxide (DMSO), clonidine (Catapress®), bretylium, ketanserin, reserpine, droperidol, atropine, phentolamine, bupivacaine, lidocaine, acetaminophen, nortriptyline (Pamelor®), amitriptyline (Elavil®), imipramine (Tofranil®), doxepin (Sinequan®), clomipramine (Anafranil®), fluoxetine (Prozac®), sertraline (Zoloft®), nefazodone (Serzone®), venlafaxine (Effexor®), trazodone (Desyrel®), bupropion (Wellbutrin®), mexiletine, nifedipine, propranolol, tramadol, lamotrigine, ziconotide, ketamine, dextromethorphan, benzodiazepines, baclofen, tizanidine and phenoxybenzamine.

Examples of additional second active agents include, but are not limited to, a steroid, a light sensitizer, an integrin, an antioxidant, an interferon, a xanthine derivative, a growth hormone, a neutrotrophic factor, a regulator of neovascularization, an anti-VEGF antibody, a prostaglandin, an antibiotic, a phytoestrogen, an anti-inflammatory compound or an antiangiogenesis compound, or a combination thereof. Specific examples include, but are not limited to, verteporfin, purlytin, an angiostatic steroid, rhuFab, interferon-2ÿ, pentoxifylline, tin etiopurpurin, motexafin lutetium, 9-fluoro-11,21-dihydroxy-16,17-1-methylethylidinebis(oxy)pregna-1,4-diene-3,20-dione, latanoprost (see U.S. Pat. No. 6,225,348), tetracycline and its derivatives, rifamycin and its derivatives, macrolides, metronidazole (U.S. Pat. Nos. 6,218,369 and 6,015,803), genistein, genistin, 6′-O-Mal genistin, 6′-O—Ac genistin, daidzein, daidzin, 6′-O-Mal daidzin, 6′-O—Ac daidzin, glycitein, glycitin, 6′-O-Mal glycitin, biochanin A, formononetin (U.S. Pat. No. 6,001,368), triamcinolone acetomide, dexamethasone (U.S. Pat. No. 5,770,589), thalidomide, glutathione (U.S. Pat. No. 5,632,984), basic fibroblast growth factor (bFGF), transforming growth factor b (TGF-b), brain-derived neurotrophic factor (BDNF), plasminogen activator factor type 2 (PAI-2), EYE101 (Eyetech Pharmaceuticals), LY333531 (Eli Lilly), Miravant, and RETISERT implant (Bausch & Lomb). All of the references cited above are incorporated herein in their entireties by reference.

Examples of additional second active agents include, but are not limited to, keratolytics, retinoids, α-hydroxy acids, antibiotics, collagen, botulinum toxin, interferon, and immunomodulatory agents. Specific examples include, but are not limited to, 5-fluorouracil, masoprocol, trichloroacetic acid, salicylic acid, lactic acid, ammonium lactate, urea, tretinoin, isotretinoin, antibiotics, collagen, botulinum toxin, interferon, corticosteroid, transretinoic acid and collagens such as human placental collagen, animal placental collagen, Dermalogen, AlloDerm, Fascia, Cymetra, Autologen, Zyderm, Zyplast, Resoplast, and Isolagen.

Examples of additional second active agents include, but are not limited to, anticoagulants, diuretics, cardiac glycosides, calcium channel blockers, vasodilators, prostacyclin analogues, endothelin antagonists, phosphodiesterase inhibitors (e.g., PDE V inhibitors), endopeptidase inhibitors, lipid lowering agents, thromboxane inhibitors, and other therapeutics known to reduce pulmonary artery pressure. Specific examples include, but are not limited to, warfarin (Coumadin®), a diuretic, a cardiac glycoside, digoxin-oxygen, diltiazem, nifedipine, a vasodilator such as prostacyclin (e.g., prostaglandin I2 (PGI2), epoprostenol (EPO, Floran®), treprostinil (Remodulin®), nitric oxide (NO), bosentan (Tracleer®), amlodipine, epoprostenol (Floran®), treprostinil (Remodulin®), prostacyclin, tadalafil (Clalis®), simvastatin (Zocor®), omapatrilat (Vanlev®), irbesartan (Avapro®), pravastatin (Pravachol®), digoxin, L-arginine, iloprost, betaprost, and sildenafil (Viagra®).

Examples of additional second active agents include, but are not limited to, anthracycline, platinum, alkylating agent, oblimersen (Genasense®), cisplatinum, cyclophosphamide, temodar, carboplatin, procarbazine, gliadel, tamoxifen, topotecan, methotrexate, taxotere, irinotecan, capecitabine, cisplatin, thiotepa, fludarabine, carboplatin, liposomal daunorubicin, cytarabine, doxetaxol, pacilitaxel, vinblastine, IL-2, GM-CSF, dacarbazine, vinorelbine, zoledronic acid, palmitronate, biaxin, busulphan, prednisone, bisphosphonate, arsenic trioxide, vincristine, doxorubicin (Doxil®), paclitaxel, ganciclovir, adriamycin, bleomycin, hyaluronidase, mitomycin C, mepacrine, thiotepa, tetracycline and gemcitabine.

Examples of additional second active agents include, but are not limited to, chloroquine, quinine, quinidine, pyrimethamine, sulfadiazine, doxycycline, clindamycin, mefloquine, halofantrine, primaquine, hydroxychloroquine, proguanil, atovaquone, azithromycin, suramin, pentamidine, melarsoprol, nifirtimox, benznidazole, amphotericin B, pentavalent antimony compounds (e.g., sodium stiboglucuronate), interfereon gamma, itraconazole, a combination of dead promastigotes and BCG, leucovorin, corticosteroids, sulfonamide, spiramycin, IgG (serology), trimethoprim, and sulfamethoxazole.

Examples of additional second active agents include, but are not limited to: antibiotics (therapeutic or prophylactic) such as, but not limited to, ampicillin, clarithromycin, tetracycline, penicillin, cephalosporins, streptomycin, kanamycin, and erythromycin; antivirals such as, but not limited to, amantadine, rimantadine, acyclovir, and ribavirin; immunoglobulin; plasma; immunologic enhancing drugs such as, but not limited to, levami sole and isoprinosine; biologics such as, but not limited to, gammaglobulin, transfer factor, interleukins, and interferons; hormones such as, but not limited to, thymic; and other immunologic agents such as, but not limited to, B cell stimulators (e.g., BAFF/BlyS), cytokines (e.g., IL-2, IL-4, and IL-5), growth factors (e.g., TGF-ÿ), antibodies (e.g., anti-CD40 and IgM), oligonucleotides containing unmethylated CpG motifs, and vaccines (e.g., viral and tumor peptide vaccines).

Examples of additional second active agents include, but are not limited to: a dopamine agonist or antagonist, such as, but not limited to, Levodopa, L-DOPA, cocaine, α-methyl-tyrosine, reserpine, tetrabenazine, benzotropine, pargyline, fenodolpam mesylate, cabergoline, pramipexole dihydrochloride, ropinorole, amantadine hydrochloride, selegiline hydrochloride, carbidopa, pergolide mesylate, Sinemet CR, and Symmetrel; a MAO inhibitor, such as, but not limited to, iproniazid, clorgyline, phenelzine and isocarboxazid; a COMT inhibitor, such as, but not limited to, tolcapone and entacapone; a cholinesterase inhibitor, such as, but not limited to, physostigmine saliclate, physostigmine sulfate, physostigmine bromide, meostigmine bromide, neostigmine methylsulfate, ambenonim chloride, edrophonium chloride, tacrine, pralidoxime chloride, obidoxime chloride, trimedoxime bromide, diacetyl monoxim, endrophonium, pyridostigmine, and demecarium; an anti-inflammatory agent, such as, but not limited to, naproxen sodium, diclofenac sodium, diclofenac potassium, celecoxib, sulindac, oxaprozin, diflunisal, etodolac, meloxicam, ibuprofen, ketoprofen, nabumetone, refecoxib, methotrexate, leflunomide, sulfasalazine, gold salts, Rho-D Immune Globulin, mycophenylate mofetil, cyclosporine, azathioprine, tacrolimus, basiliximab, daclizumab, salicylic acid, acetylsalicylic acid, methyl salicylate, diflunisal, salsalate, olsalazine, sulfasalazine, acetaminophen, indomethacin, sulindac, mefenamic acid, meclofenamate sodium, tolmetin, ketorolac, dichlofenac, flurbinprofen, oxaprozin, piroxicam, meloxicam, ampiroxicam, droxicam, pivoxicam, tenoxicam, phenylbutazone, oxyphenbutazone, antipyrine, aminopyrine, apazone, zileuton, aurothioglucose, gold sodium thiomalate, auranofin, methotrexate, colchicine, allopurinol, probenecid, sulfinpyrazone and benzbromarone or betamethasone and other glucocorticoids; and an antiemetic agent, such as, but not limited to, metoclopromide, domperidone, prochlorperazine, promethazine, chlorpromazine, trimethobenzamide, ondansetron, granisetron, hydroxyzine, acetylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxypemdyl, pipamazine, scopolamine, sulpiride, tetrahydrocannabinol, thiethylperazine, thioproperazine, tropisetron, and a mixture thereof.

Examples of additional second active agents include, but are not limited to, immunomodulatory agents, immunosuppressive agents, antihypertensives, anticonvulsants, fibrinolytic agents, antiplatelet agents, antipsychotics, antidepressants, benzodiazepines, buspirone, amantadine, and other known or conventional agents used in patients with CNS injury/damage and related syndromes. Specific examples include, but are not limited to: steroids (e.g., glucocorticoids, such as, but not limited to, methylprednisolone, dexamethasone and betamethasone); an anti-inflammatory agent, including, but not limited to, naproxen sodium, diclofenac sodium, diclofenac potassium, celecoxib, sulindac, oxaprozin, diflunisal, etodolac, meloxicam, ibuprofen, ketoprofen, nabumetone, refecoxib, methotrexate, leflunomide, sulfasalazine, gold salts, RHo-D Immune Globulin, mycophenylate mofetil, cyclosporine, azathioprine, tacrolimus, basiliximab, daclizumab, salicylic acid, acetylsalicylic acid, methyl salicylate, diflunisal, salsalate, olsalazine, sulfasalazine, acetaminophen, indomethacin, sulindac, mefenamic acid, meclofenamate sodium, tolmetin, ketorolac, dichlofenac, flurbinprofen, oxaprozin, piroxicam, meloxicam, ampiroxicam, droxicam, pivoxicam, tenoxicam, phenylbutazone, oxyphenbutazone, antipyrine, aminopyrine, apazone, zileuton, aurothioglucose, gold sodium thiomalate, auranofin, methotrexate, colchicine, allopurinol, probenecid, sulfinpyrazone and benzbromarone; a cAMP analog including, but not limited to, db-cAMP; an agent comprising a methylphenidate drug, which comprises 1-threo-methylphenidate, d-threo-methylphenidate, dl-threo-methylphenidate, 1-erythro-methylphenidate, d-erythro-methylphenidate, dl-erythro-methylphenidate, and a mixture thereof; and a diuretic agent such as, but not limited to, mannitol, fuirosemide, glycerol, and urea.

Examples of additional second active agents include, but are not limited to, a tricyclic antidepressant agent, a selective serotonin reuptake inhibitor, an antiepileptic agent (gabapentin, pregabalin, carbamazepine, oxcarbazepine, levitiracetam, topiramate), an antiaryhthmic agent, a sodium channel blocking agent, a selective inflammatory mediator inhibitor, an opioid agent, a second immunomodulatory compound, a combination agent, and other known or conventional agents used in sleep therapy. Specific examples include, but are not limited to, Neurontin, oxycontin, morphine, topiramate, amitryptiline, nortryptiline, carbamazepine, Levodopa, L-DOPA, cocaine, α-methyl-tyrosine, reserpine, tetrabenazine, benzotropine, pargyline, fenodolpam mesylate, cabergoline, pramipexole dihydrochloride, ropinorole, amantadine hydrochloride, selegiline hydrochloride, carbidopa, pergolide mesylate, Sinemet CR, Symmetrel, iproniazid, clorgyline, phenelzine, isocarboxazid, tolcapone, entacapone, physostigmine saliclate, physostigmine sulfate, physostigmine bromide, meostigmine bromide, neostigmine methylsulfate, ambenonim chloride, edrophonium chloride, tacrine, pralidoxime chloride, obidoxime chloride, trimedoxime bromide, diacetyl monoxim, endrophonium, pyridostigmine, demecarium, naproxen sodium, diclofenac sodium, diclofenac potassium, celecoxib, sulindac, oxaprozin, diflunisal, etodolac, meloxicam, ibuprofen, ketoprofen, nabumetone, refecoxib, methotrexate, leflunomide, sulfasalazine, gold salts, RHo-D Immune Globulin, mycophenylate mofetil, cyclosporine, azathioprine, tacrolimus, basiliximab, daclizumab, salicylic acid, acetylsalicylic acid, methyl salicylate, diflunisal, salsalate, olsalazine, sulfasalazine, acetaminophen, indomethacin, sulindac, mefenamic acid, meclofenamate sodium, tolmetin, ketorolac, dichlofenac, flurbinprofen, oxaprozin, piroxicam, meloxicam, ampiroxicam, droxicam, pivoxicam, tenoxicam, phenylbutazone, oxyphenbutazone, antipyrine, aminopyrine, apazone, zileuton, aurothioglucose, gold sodium thiomalate, auranofin, methotrexate, colchicine, allopurinol, probenecid, sulfinpyrazone, benzbromarone, betamethasone and other glucocorticoids, metoclopromide, domperidone, prochlorperazine, promethazine, chlorpromazine, trimethobenzamide, ondansetron, granisetron, hydroxyzine, acetylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxypemdyl, pipamazine, scopolamine, sulpiride, tetrahydrocannabinol, thiethylperazine, thioproperazine, tropisetron, and a mixture thereof.

Examples of additional second active agents include, but are not limited to: interleukins, such as IL-2 (including recombinant IL-II (“rIL2”) and canarypox IL-2), IL-10, IL-12, and IL-18; interferons, such as interferon alfa-2a, interferon alfa-2b, interferon alfa-n1, interferon alfa-n3, interferon beta-I a, and interferon gamma-I b; and G-CSF; hydroxyurea; butyrates or butyrate derivatives; nitrous oxide; HEMOXIN™ (NIPRISAN™; see U.S. Pat. No. 5,800,819, incorporated by reference herein in its entirety); Gardos channel antagonists such as clotrimazole and triaryl methane derivatives; Deferoxamine; protein C; and transfusions of blood, or of a blood substitute such as Hemospan™ or Hemospan™ PS (Sangart).

Administration of a Compound and a second active agent to a patient can occur simultaneously or sequentially by the same or different routes of administration. The suitability of a particular route of administration employed for a particular active agent will depend on the active agent itself (e.g., whether it can be administered orally without decomposing prior to entering the blood stream) and the disease being treated. A preferred route of administration for Compounds is oral. Preferred routes of administration for the second active agents or ingredients of the invention are known to those skilled in the art. See, e.g., Physicians' Desk Reference, 1755-1760 (56th ed., 2002).

In one embodiment, the second active agent is administered intravenously or subcutaneously and once or twice daily in an amount of from about 1 to about 1000 mg, from about 5 to about 500 mg, from about 10 to about 350 mg, or from about 50 to about 200 mg. The specific amount of the second active agent will depend on the specific agent used, the type of disease being treated or managed, the severity and stage of disease, and the amount(s) of a Compound and any optional additional active agents concurrently administered to the patient.

### 5 EXAMPLES

### 5.1 Biological Assays

### 5.1.1 PBMC Culture and ELISA for TNF-α

Peripheral blood mononuclear cells (PBMC) are prepared by density centrifugation on Ficoll-Hypaque. PBMC, re-suspended at 1×106/ml in complete RPMI-1640 medium/10% fetal calf serum, are stimulated with LPS (1 μg/ml; Escherichia coli serotype 0127:B8; Sigma) in 24-well plates by incubation at 37° C. in 5% CO₂ for 24 h± with Compounds (0.1-100 μM). Cell-free supernatants are collected and stored in aliquots at −70° C. until assayed by ELISA. Supernatants are assayed for TNFα using an assay procedure and reagents provided by R&D Systems (Minneapolis, Minn.).

### 5.1.2 TNF-α inhibition

Rat and Human PBMC LPS-Induced TNF-α Assay

Lipopolysaccharide (LPS) is an endotoxin produced by gram-negative bacteria such as E. coli which induces production of many pro-inflammatory cytokines, including TNF-α. In PBMC, the TNF-α production in response to LPS is derived from monocytes, which comprise approximately 5-20% of the total PBMC. Compounds are tested for their ability to inhibit LPS-induced TNF-α production from either rat or human PBMC as previously described (Muller et al. J. Med. Chem. 39:3238 (1996)). PBMC from normal donors are obtained by Ficol Hypaque (Pharmacia, Piscataway, MJ, USA) density centrifugation. Cells are cultured in RPMI (Life Technologies, Grand Island, N.Y., USA) supplemented with 10% AB±human serum (Gemini Bio-products, Woodland, Calif., USA), 2 mM L-glutamine, 100 U/ml penicillin and 100 μg/ml streptomycin (Life Technologies).

PBMC (2×10⁵ cells) are plated in 96-well flat-bottomed Costar tissue culture plates (Corning, N.Y., USA) in triplicate. Cells are stimulated with LPS (Sigma, St. Louis, Mo., USA) at 100 ng/ml in the absence or presence of compounds. Compounds are dissolved in DMSO (Sigma) and further dilutions are done in culture medium immediately before use. The final DMSO concentration in all samples is 0.25%. Compounds are added to cells 1 hour before LPS stimulation. Cells are incubated for 18-20 hours at 37° C. in 5% CO₂ and supernatants are then collected, diluted with culture medium and assayed for TNF-α levels by ELISA (Endogen, Boston, Mass., USA). In one embodiment, LPS-induced TNF-α IC₅₀ of Compounds ranges between about 1 nM to about 1 μM.

Rat LPS-Induced Serum TNF-α Inhibition

Compounds are tested in the Rat LPS/TNF assay that is performed as follows: Female CD-IGS rats (200 g from Charles River Laboratories, Hollister, Calif.) receive LPS in saline via i.v. injection (20 μg; Escherichia coli 055:B5). At 90 minutes, a terminal bleed is obtained from the abdominal vena cava, and the serum is recovered. Samples are analyzed for mouse TNF-α by using an ELISA (BioSource International, Camarillo, Calif.). Compounds are dosed (p.o. in 0.5% CMC/0.25% Tween vehicle) prior to LPS challenge. In one embodiment, LPS-induced TNF-α ED₅₀ of Compounds ranges between about 0.3 mg/kg to about 30 mg/kg.

### 5.1.3 Cytokine Profiling

Human PBMC isolated as described above are stimulated with either 100 ng/ml LPS to activate the monocyte fraction of 1 μg/ml SEB and 2 μg/ml Anti-CD28 to activate the T-lymphocyte fraction. Cultures are incubated 18-24 h and media removed and analyzed for expression of IL-1β, IL-6, IL-8, IL-12, MIP1α, RANTES and TNFα for LPS stimulated samples, and IL-1β, IL-2, IL-6, IL-8, IL-10, IFNγ, MCP-1 for SEB/CD28 stimulated samples. Cytokines are quantified by multiplex analysis using the Biorad Bioplex analyzer based on Luminex technology. In one embodiment, inhibitory activity of Compounds is limited to inhibition of TNF-α and IL-12 in LPS stimulated PBMC.

### 5.1.4 PDE4 Inhibition

PDE4 (U937 Cell-Derived) Enzyme Assay

PDE4 enzyme is purified from U937 human monocytic cells by gel filtration chromatography as previously described (Muller et al. Bioorg. & Med Chem Lett 8:2669-2674 (1998)). Phosphodiesterase reactions are carried out in 50 mM Tris HCl pH 7.5, 5 mM MgCl₂, 1 μM cAMP, 10 nM [³H]-cAMP for 30 minutes at 30° C., terminated by boiling, treated with 1 mg/ml snake venom and separated using AG-1XS ion exchange resin (BioRad) as described (Id). Reactions consumed less than 15% of available substrate. In one embodiment, PDE4 IC₅₀ of compounds ranges between about 4 nM to about 1 μM.

### 5.1.5 PDE Selectivity

PDE1, 2, 3, 5 and 6 Enzyme Assays

The phosphodiesterase selectivity of 2,5-Diamino-4-(4-chloro-phenyl)-thieno[2,3-d]pyrimidine-6-carboxylic acid amide was assessed by testing against bovine PDE1, human PDE2, PDE3 and PDE5 from human platelets and PDE6 from bovine retinal rod outer segments (MDS Pharma Services). Results for the representative compound are set forth below.

| PDE inhibition | |
|---|---|
| PDE4 IC₅₀ (from U937 cells) | 120 nM |
| PDE1 (% inhibit at 10 μM) | 18% |
| PDE2 (% inhibit at 10 μM) | 8% |
| PDE3 (% inhibit at 10 μM) | 14% |
| PDE5 (% inhibit at 10 μM) | −2% |
| PDE6 (% inhibit at 10 μM) | 8% |

### 5.1.6 Wild Type B-RAF ³³P Assay

Wild-type B-RAF is obtained from Upstate. Substrate (HIS-MEK1 K97M) is prepared by Celgene Corp. as follows. Human MEK1 is cloned into a pET151 vector. Lysine-97 of the MEK1 sequence is mutated to methionine to generate a kinase-dead version of the MEK1 protein, to ensure that the phosphorylation of the MEK1 substrate is a result of B-RAF kinase activity and not MEK1 auto-phosphorylation. The MEK1 K97M clone is transformed into a BL21 (DE3) cell line and tested for expression. The culture is grown at 30° C. up to an optical density (OD) of 0.8 and induced with 0.1 mM IPTG at 18° C. overnight. Cell pellets are re-suspended in lysis buffer (20 mM Tris-HCl, 500 mM NaCl, 10 mM Imidazole, 0.1% Brij-35, 10% Glycerol, Roche protease inhibitor tablets, 5 mM BME, pH 7.5) and lysed using a homogenizer. Purification is performed using a nickel charged fast flow column on an AKTA purifier. All MEK1 protein is pooled and dialyzed into a storage buffer (50 mM Tris pH 7.5, 150 mM NaCl, 0.1 mM EGTA, 0.03% Brij-35, protease inhibitor tablets, 0.1% BME, 10% glycerol) and concentrated down to approximately 3.0 mg/ml.

Test compound is diluted in 100% DMSO (600 μM). Enzyme mixture is prepared as 5.71 nM B-RAF in assay buffer (50 mM Tris-HCl, 0.1 mg/ml BSA, 0.03% Brij 35, 1 mM DTT, pH 7.6). Substrate mixture is prepared as 7.5 μM HIS-MEK1 K97M in assay buffer. ATP mixture is prepared as 300 μM ATP, 75 mM MgCl₂, 0.05 μCi/μl ³³P ATP in assay buffer.

In a 96 well assay plate (Falcon Polylpropylene well U-bottom), the following are added to each well: 2.5 μl of test compound solution (serial dilutions), 20 μl of substrate mixture and 17.5 μl of enzyme mixture. The plate is incubated for at least 15 minutes. 10 μl of ATP mixture is added and the plate is incubated at room temperature for about 60 minutes. 80 μl of 12.5% TCA is added to each well, and the plate is incubated for at least 20 minutes. The contents of the plate is transferred to a glass fiber filter plate (Millipore) using a Packard Harvester (washing 10× with 1×PBS). 50 μl of Scintillation-20 fluid is added to each well. The plate is read using a Packard TopCount. This assay demonstrates the ability of a Compound to inhibit wild-type B-RAF.

### 5.1.7 V600E Mutant B-RAF ³³P Assay

V600E mutant B-RAF is obtained from Upstate. Substrate (HIS-MEK1 K97M) is prepared by Celgene Corp. as described above.

Test compound is diluted in 100% DMSO (600 μM). Enzyme mixture is prepared as 5.71 nM V600E mutant B-RAF in assay buffer (50 mM Tris-HCl, 0.1 mg/ml BSA, 0.03% Brij 35, 1 mM DTT, pH 7.6). Substrate mixture is prepared as 7.5 μM HIS-MEK1 K97M in assay buffer. ATP mixture is prepared as 300 μM ATP, 75 mM MgCl₂, 0.05 μCi/μl ³³P ATP in assay buffer.

In a 96 well assay plate (Falcon Polylpropylene well U-bottom), the following are added to each well: 2.5 μl of test compound solution (serial dilutions), 20 μl of substrate mixture and 17.5 μl of enzyme mixture. The plate is incubated for at least 15 minutes. 10 μl of ATP mixture is added and the plate is incubated at room temperature for about 60 minutes. 80 μl of 12.5% TCA is added to each well, and the plate is incubated for at least 20 minutes. The contents of the plate is transferred to a glass fiber filter plate (Millipore) using a Packard Harvester (washing 10× with 1×PBS). 50 μl of Scintillation-20 fluid is added to each well. The plate is read using a Packard TopCount. This assay demonstrates the ability of a Compound to inhibit V600E mutant B-RAF.

### 5.1.8 [³H] Rolipram Binding Assay

The ability of compounds to displace Rolipram from the high affinity binding site in mouse brain is assessed using the Rolipram radioligand binding assay (MSD Pharma Services) as described previously (Duplantier et al., J. Med. Chem. 39(1): 120-125 (1996)). In one embodiment, rolipram binding IC₅₀ of Compounds ranges between about 5 nM to about 0.1 μM.

### 5.1.9 Animal Disease Models

Efficacy of Compounds can be demonstrated in both the murine collagen-induced arthritis model (methods described previously in Gerlag et al., J. Immunol. 165(3):1652-1658 (2000)) of rheumatoid arthritis and the murine ovalbumin-induced asthma model (methods previously described using cockroach allergen by Campbell et al., J. Immunol. 161(12):7047-7053 (1998)). Inhibition of clinical paw scoring in the CIA model of Compounds can be assayed and compared to the positive control, Indomethacin (2 mg/kg). Inhibition of airway hyper-responsiveness to Methacholine challenge (3 mg/kg; i.v.) by Compounds can be assayed using the OVA asthma model.

### 5.1.10 7 Day Toxicological Assessment

Toxicity of a Compound is assessed over 7 days oral dosing (q.d.) in female CD-IGS rats (200 g; Charles River). Gross pathological, clinical or histological toxicity are screened for in organs (mesentery tissue, liver and kidney) following 7 day oral dosing with a Compound. In one embodiment, therapeutic index vs. ED₅₀ in TNF-α induced LPS release in rats of Compounds is >20.

### 5.1.11 A375 pMEK1/2 Meso Scale Assay

On the afternoon of Day 1, cells are plated in a 96-well flat bottom cell culture plate at about 50,000 cells/well in a volume of 100 μl. On the morning of Day 2, serial dilutions of test compound(s) are added to the plate. The plate is incubated for about two hours at 37° C., 0.5% CO₂. The plate is blocked about five minutes before the compound incubation step is completed by adding 150 μl/well of MSD (Meso Scale Discovery) blocking solution-A (prepared with 20 ml 1× Tris wash buffer (25 ml of 10× Tris buffer+225 ml deionized water) and 600 mg MSD blocker A) and incubating with vigorous shaking at room temperature for one hour. The plate is washed 4× with 1× Tris wash buffer.

Cells are harvested and lysates prepared by removing medium, washing 1× with ice-cold PBS (calcium and magnesium free), adding 50 μl/well Complete Tris buffer and incubating with shaking at 4° C. for one hour. Lysate samples (25 μl) are added to an MSD Multi-Spot plate and incubated with vigorous shaking at room temperature for two hours. The plate is washed 1× with Tris wash buffer.

25 μl/well of 10 mM detection antibody (Anti-pMEK1/2 antibody; SULFO-TAG labeled, light sensitive) in 3 ml of cold antibody dilution buffer (1 ml blocking solution-A, 1.82 ml 1× Tris wash buffer, 150 μl 2% MSD blocker D-M and 30 μl 10% MSD blocker D-R) is added and the plate is incubated with vigorous shaking at room temperature in the dark for one hour. The plate is washed 1× with Tris wash buffer.

150 μl/well of 1× MSD Read buffer T (with surfactant) is added to the plate and the plate is read using a MSD Sector instrument.

### 5.1.12 Alamar Blue Viability Assay

A375 cells (ATCC # CRL-1619; Passage 4˜15; doubling time is roughly 18 hours) are used. Culture medium is DMEM (high glucose, with L-glutamine)+10% FBS+Pen/Strep. Cells are passed every three days at 1:20.

200 μl of medium only is added into all wells along the edges of a 96-well flat bottom plate and 180 μl of cell suspension (containing 1,000 cells) per well is added into the plate with a Low Evaporation Lid (Costar Cat # 3595). The cells are cultured overnight. Compounds and controls are diluted according to standard compound dilution protocol described below. From 30 mM stock solution, 10 mM, 3 mM, 1 mM, and 0.3 mM solutions in 100% DMSO are prepared using the following serial dilution protocol:

| Starting | | | | Final | |
|---|---|---|---|---|---|
| Concentration | | Dilution | | Concentration | |
| 30 | mM | 1:3 | (7.5 μl + 15 μl DMSO) | 10 | mM |
| 10 | mM | 1:3.3 | (7.5 μl + 17.5 μl DMSO) | 3 | mM |
| 3 | mM | 1:3 | (7.5 μl + 15 μl DMSO) | 1 | mM |
| 1 | mM | 1:3.3 | (7.5 μl + 17.5 μl DMSO) | 0.3 | mM |

The 30, 10, 3, 1 and 0.3 mM solutions are diluted 1:50 (DMSO:medium) by adding 4 μl of each to 196 μl of culture medium. This yields 600 μM, 200 μM, 60 μM, 20 μM, and 6 μM secondary stock solutions. 4 μl of DMSO are added to 196 μl medium for the DMSO control as well. 20 μl of compound dilutions, including the DMSO control locations (Row G, Columns 2-10 are DMSO control wells), are added in triplicate to the cells in the 96-well plate using a repeat pipettor (see plate layout below). This yields final concentrations of 60 μM, 20 μM, 6 μM, 2 μM, and 0.6 μM containing 0.2% DMSO in each well. The plate is gently tapped to evenly distribute compound within the well. The plate is incubated at 5% CO₂ at 37° C. for 3 days.

| Compound #1 | | | Compound #2 | | | Compound #3 | | |
|---|---|---|---|---|---|---|---|---|
| 60 uM | 60 uM | 60 uM | 60 uM | 60 uM | 60 uM | 60 uM | 60 uM | 60 uM |
| 20 uM | 20 uM | 20 uM | 20 uM | 20 uM | 20 uM | 20 uM | 20 uM | 20 uM |
| 6 uM | 6 uM | 6 uM | 6 uM | 6 uM | 6 uM | 6 uM | 6 uM | 6 uM |
| 2 uM | 2 uM | 2 uM | 2 uM | 2 uM | 2 uM | 2 uM | 2 uM | 2 uM |
| .6 uM | .6 uM | .6 uM | .6 uM | .6 uM | .6 uM | .6 uM | .6 uM | .6 uM |
| DMSO | DMSO | DMSO | DMSO | DMSO | DMSO | DMSO | DMSO | DMSO |

20 μl of Alamar Blue (Biosource #DAL-1100) is added to each well. The plate is incubated in 5% CO2 incubator for 4 hours. Fluorescence is measured with excitation wavelength at 530 nm and emission wavelength at 590 nm using a Victor 2 multilabel plate reader (Perkin Elmer). Data is saved with plate format as a Text file and entered into ActivityBase (Cell-based SAR; Prolif-Alamar; MTT 5 plates unfixed fit) to calculate the percent of DMSO values for each compound set of triplicate wells and IC₅₀ curves are plotted.

Compounds of Table 1 were found to have the following values in the V600E Mutant B-RAF ³³P, the A375 pMEK1/2 Meso Scale and the Alamar Blue Viability screening assays.

| | V600E Mutant | Meso Scale | Alamar Blue |
|---|---|---|---|
| Compound | IC₅₀ (μM) | IC₅₀ (μM) | IC₅₀ (μM) |
| 5 | ***** | ***** | **** |
| 162 | ***** | ***** | **** |
| 163 | ***** | **** | ** |
| 164 | ***** | **** | *** |
| 165 | ***** | ***** | ***** |
| 168 | ***** | ND | *** |
| 169 | ***** | ***** | *** |
| 170 | ** | ND | * |
| 171 | ***** | ***** | *** |
| 172 | ** | * | * |
| 173 | ***** | ***** | ***** |
| 174 | ***** | ***** | ***** |
| 175 | ***** | ***** | **** |
| 176 | ***** | ***** | ***** |
| 177 | ***** | ND | **** |
| 178 | ***** | ND | **** |
| 179 | ** | ND | **** |
| 180 | ***** | ND | **** |
| 181 | ** | ND | **** |
| 182 | ** | ND | ** |
| 183 | ***** | ND | ** |
| 184 | ***** | ND | *** |
| 185 | ***** | ***** | ***** |
| 186 | ***** | ND | *** |
| 187 | ***** | ND | *** |
| 188 | ***** | ***** | **** |
| 189 | ***** | *** | ***** |
| 190 | ***** | ***** | ***** |
| 191 | ***** | ***** | ***** |
| 192 | ***** | ***** | ***** |
| 193 | ***** | ND | *** |
| 194 | ***** | * | * |
| 195 | ***** | *** | ***** |
| 196 | ***** | ** | **** |
| 197 | ***** | ND | **** |
| 198 | ***** | ***** | **** |
| 199 | ***** | ***** | ***** |
| 200 | ***** | ***** | **** |
| 201 | ***** | ***** | **** |
| 202 | ***** | *** | **** |
| 203 | ***** | ***** | **** |
| 204 | ***** | **** | *** |
| 205 | ***** | *** | *** |
| 206 | ***** | *** | *** |
| 207 | ***** | **** | **** |
| 208 | ***** | ***** | **** |
| 209 | ***** | ND | **** |
| 210 | ***** | ***** | ***** |
| 211 | ***** | ND | **** |
| 212 | ***** | ***** | **** |
| 213 | ***** | ND | * |
| 214 | ***** | ***** | ***** |
| 215 | ***** | ND | **** |
| 216 | ***** | ND | **** |
| 217 | ***** | **** | ***** |
| 218 | ***** | ** | **** |
| 219 | ***** | * | * |
| 220 | ***** | **** | *** |
| 221 | ***** | ND | **** |
| 222 | ***** | *** | *** |
| 223 | ***** | ND | ND |
| 224 | ***** | ***** | **** |
| 225 | ***** | ***** | ***** |
| 226 | ***** | ***** | ***** |
| 227 | ***** | ND | ***** |
| 228 | ***** | ***** | **** |
| 229 | ***** | ND | **** |
| 230 | ***** | **** | *** |
| 231 | ***** | ***** | ***** |
| 232 | ***** | ***** | ***** |
| 233 | ***** | ND | **** |
| 234 | ***** | ***** | ** |
| 235 | ***** | ND | *** |
| 236 | ***** | ND | ** |
| 237 | **** | ND | * |
| 238 | ***** | **** | ND |
| 239 | ***** | ***** | ***** |
| 240 | ***** | ***** | *** |
| 241 | ***** | ***** | **** |
| 242 | ***** | ***** | **** |
| 243 | ***** | ***** | ***** |
| 244 | ***** | ND | ** |
| 245 | ***** | ND | * |
| 246 | **** | ND | * |
| 247 | ***** | ***** | ***** |
| 248 | ***** | **** | **** |
| 249 | ***** | ***** | ***** |
| 250 | ***** | ***** | ***** |
| 251 | ***** | ***** | ***** |
| 252 | ***** | ***** | ***** |
| 253 | ***** | ***** | ***** |
| 254 | ***** | ND | * |
| 255 | ***** | ND | * |
| 256 | ***** | ***** | ***** |
| 257 | ***** | ***** | ***** |
| 258 | ***** | ***** | ***** |
| 259 | ***** | ***** | ***** |
| 261 | ***** | ***** | **** |
| 262 | ***** | ***** | ***** |
| 263 | ND | ND | **** |
| 264 | ***** | ***** | ***** |
| 265 | ***** | *** | *** |
| 266 | * | ND | **** |
| 267 | ***** | ND | **** |
| 268 | ***** | ND | ** |
| 269 | ***** | * | * |
| 270 | ***** | *** | ***** |
| 271 | ***** | ** | **** |
| 272 | ***** | ND | **** |
| 273 | ***** | ***** | **** |
| 274 | ***** | ***** | ***** |
| 275 | ***** | **** | **** |
| 276 | ***** | * | **** |
| 277 | ND | ND | ***** |
| 278 | **** | ND | ** |
| 279 | ** | ND | ***** |
| 280 | ***** | ***** | ***** |
| 281 | ***** | ND | **** |
| 282 | ***** | **** | **** |
| 283 | ND | ND | **** |
| 284 | ** | ND | **** |
| 285 | ***** | ND | **** |
| 286 | ***** | ND | **** |
| 287 | ND | ND | ***** |
| 288 | ***** | ND | **** |
| 289 | ND | ND | **** |
| 290 | ND | ND | **** |
| 291 | ***** | ***** | **** |
| 292 | ***** | * | **** |
| 293 | ***** | ND | **** |
| 294 | ND | ND | **** |
| 295 | ***** | ***** | ***** |
| 296 | ***** | ** | **** |

In the table set forth above, the following system is used:
***** = 0.1-10 μM,
**** = 10.01-20 μM,
*** = 20.01-30 μM,
** = 30.01-60 μM,
* = >60 μM.
“ND” means that the compound was not tested in that particular assay.

### 5.2 Synthesis of Illustrative Examples of the Compounds

### 5.2.1 2-Amino-4-(4-chlorophenyl)-thieno[2,3-d]pyrimidine-6-carboxylic acid methyl amide

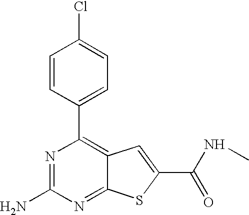

Step a: 4,6-Dichloro-2-methylsulfanyl-pyrimidine-5-carbaldehyde. POCl₃ (70 mL, 0.75 mol) was cooled to −5° C. and DMF (23 mL, 0.3 mol) was added slowly. The resultant mixture was allowed to stand at 20° C. for 1 hour. 4,6-dihydroxy-2-methyl-mercaptopyrimidine (15.8 g., 0.1 mol) was added using a solid addition funnel. The reaction mixture was first stirred at room temperature for 30 minutes and then heated at reflux for 3 hours. Following removal of excess POCl₃ and DMF in vacuo, the residue was poured into ice. The solid obtained was filtered and washed with cold water. After treating the solid with hexanes (sonication). the desired product was obtained as a white solid (9.3 g, 42%). ¹H NMR (CDCl₃, 300 MHz) δ 10.34 (s, 1H), 2.64 (s, 3H); CHN Calc'd for C₆H₄Cl₂N₂OS: C, 32.30; H, 1.81; N, 12.56. Found: C, 32.44; H, 1.69; N, 12.51.

Step b: (6-Chloro-5-formyl-2-methylsulfanyl-pyrimidin-4-ylsulfanyl)-acetic acid ethyl ester. To a solution of pyrimidine (1.55 g, 7.0 mmol) from step (a) in THF (20 mL) was added Et₃N (1.5 equiv, 10.5 mmol, 1.5 mL). The solution was then cooled to 0° C., followed by dropwise addition of ethyl-2-mercapto-acetate (1 equiv, 0.77 mL). The mixture was stirred for 1 h at 0° C. After this time, the reaction was concentrated, triturated with hexanes and filtered. The mother liquor was concentrated and subjected to silica gel:chromatography (88:12 hexanes/ethyl acetate). Concentration of the desired fractions afforded 1.6 g of the desired compound as a white solid (5.2 mmol, 75%). ¹H NMR (CDCl₃, 300 MHz) δ 10.43 (s, 1H), 4.22 (q, 2H, J=7.2 Hz), 3.90 (s, 2H), 2.60 (s, 3H), 1.28 (t, 3H, J=7.2 Hz). CHN Calc'd for C₁₀H₁₁ClN₂O₃S₂: C, 39.15; H, 3.61; N, 9.13. Found: C, 39.29; H, 3.50; N, 9.05.

Step c: 4-Chloro-2-methylsulfanyl-thieno[2,3-d]pyrimidine-6-carboxylic acid ethyl ester. To a solution of chloropyrimidine (1.0 g, 3.3 mmol) from step (b) in THF/DMF (19:1 v/v; 15 mL) was added K₂CO₃ (2 equiv, 9.80 mmol, 1.35 g). The mixture was heated in a microwave reactor at 140° C. for 25 minutes. The slurry was then filtered through a coarse-frit glass funnel and then concentrated. The resulting oil was subjected to column:chromatography (95:5 hexanes/ethyl acetate). Concentration of the desired fractions afforded 0.5 g (33%) of the desired compound as a white solid. ¹H NMR (CDCl₃, 300 MHz) δ 8.00 (s, 1H), 4.45 (q, 2H, J=6.3 Hz), 2.66 (s, 3H), 1.44 (t, 3H, J=6.3 Hz). CHN Calc'd for C₁₀H₉ClN₂O₂S₂: C, 41.59; H, 3.14; N, 9.70. Found: C, 41.78; H, 2.89; N, 9.56.

Step d: 4-(4-Chlorophenyl)-2-methylsulfanyl-thieno[2,3-d]pyrimidine-6-carboxylic acid ethyl ester. Chloropyrimidine (0.5 g, 1.75 mmol) from step (c) was dissolved in ethylene glycol dimethyl ether (5 mL). To this solution was added 4-chlorophenylboronic acid (1.2 equiv, 2.1 mmol, 0.33 g), PdCl₂dppf (5 mol %, 71 mg), and K₃PO₄ (4 equiv, 7.0 mmol, 1.48 g). The mixture was heated in a sealed flask for 24 h at 85° C. The reaction mixture was then cooled to room temperature, filtered through Celite®, and then concentrated. The residue was subjected to column chromatography (95:5 hexanes/ethyl acetate). Concentration of the desired fractions afforded 0.44 g of the desired compound as a white solid (69%). ¹H NMR (CDCl₃, 300 MHz) δ 8.12 (s, 1H), 7.90 (d, 2H, J=8.4 Hz), 7.55 (d, 2H, J=8.4 Hz), 4.30 (q, 2H, J=7.2 Hz), 2.69 (s, 3H), 1.43 (t, 3H, J=7.2 Hz).

Step e: 2-Amino-4-(4-chlorophenyl)-thieno[2,3-d]pyrimidine-6-carboxylic acid methyl amide. Thiomethylpyrimidine (0.44 g, 1.21 mmol) from step (d) was dissolved in THF (10 mL). To this solution was added LiOH (4 equiv, 4.84 mmol, 0.2 g) in H₂O (10 mL). The mixture was stirred at 65° C. for 24 hours. After this time, the mixture was acidified with 5% HCl (aq, 10 mL) and extracted with CH₂Cl₂ (3×10 mL). The organic extracts were combined, dried (Na₂SO₄), filtered, and then concentrated. The crude acid (0.1 g, 0.3 mmol) was dissolved in DMF (2 mL), followed by addition of EDCI (1.4 equiv, 80 mg), HOBt (1 equiv, 40 mg), and methylamine (2M in THF, 0.3 mL). The mixture was stirred 24 hours. After this time, the reaction mixture was partitioned between 1% NaOH (aq, 10 mL) and CH₂Cl₂ (10 mL). The mixture was shaken and separated. The organic layer was dried (Na₂SO₄) and then filtered. To the crude amide in dichloromethane was added m-CPBA (2 equiv, 0.11 g). The solution was stirred at room temperature for 2 hours. The reaction mixture was then washed with 5% NaHSO₃ (aq, 20 mL) and then with 10% NaHCO₃ (aq, 20 mL). The organic layer was dried (Na₂SO₄), filtered, and then concentrated. The residue was dissolved in CH₃CN (10 mL) and NH₄OH (5 mL). This mixture was stirred at 60° C. for 24 hours. The solution was then concentrated to remove CH₃CN and then diluted with CH₂Cl₂ (10 mL) and H₂O (10 mL). The mixture was shaken and separated. The organic layer was dried (Na₂SO₄), filtered, and then concentrated. The residue was purified using preparatory reverse-phase HPLC using a 20%-70% CH₃CN/H₂O gradient. The desired fractions were combined, made alkaline with 10% NaHCO₃ (aq), and washed with CH₂Cl₂ (10 mL). The organic layer was dried (Na₂SO₄), filtered, and then concentrated to afford the title compound as a white solid. ¹H NMR (CDCl₃, 400 MHz) δ 7.80 (d, 2H, J=8.4 Hz), 7.73 (s, 1H), 7.50 (d, 2H, J=8.4 Hz), 5.95 (br s, 1H), 5.26 (br s, 2H), 3.02 (d, 3H, J=4.8 Hz). LCMS (M+H=318.9 (6.74 minutes).

### 5.2.2 4-(3,4-Dichlorophenyl)-2-amino-thieno[2,3-d]pyrimidine-6-carboxylic acid amide

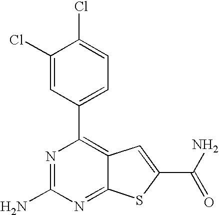

Step a: 4-(3,4-Dichlorophenyl)-2-methylsulfonyl-thieno[2,3-d]pyrimidine-6-carboxylic acid ethyl ester. 4-(3,4-Dichlorophenyl)-2-methylsulfanyl-thieno[2,3-d]pyrimidine-6-carboxylic acid ethyl ester (0.43 g, 1.5 mmol; prepared according to Example 5.2.1, Step d) was dissolved in CHCl₃ (20 mL) and m-CPBA (2 equiv, 0.34 g) was added. The mixture was stirred for 2 hours. The solution was then washed with NaHSO₃ (saturated aqueous solution) followed by NaHCO₃ (10% aq). The organic layer was dried (Na₂SO₄), filtered, and then concentrated.

Step b: 4-(3,4-Dichlorophenyl)-2-amino-thieno[2,3-d]pyrimidine-6-carboxylic acid ethyl ester. The crude sulfone from step (a) was then dissolved in THF (10 mL) and to this solution was added NH₃ (aq, 2 mL). The mixture was stirred in a sealed flask at 65° C. for 12 hours. On cooling, the mixture was concentrated and the resulting residue then triturated with MeOH (3 mL) to afford 4-(3,4-Dichlorophenyl)-2-amino-thieno[2,3-d]pyrimidine-6-carboxylic acid ethyl ester as a white powder.

Step c: 4-(3,4-Dichlorophenyl)-2-amino-thieno[2,3-d]pyrimidine-6-carboxylic acid amide. The material from step (b) was suspended in MeOH (10 mL) and KCN (10 mol %) added. The reaction mixture was then cooled in an ice bath while NH₃ (g) was bubbled through the solution until saturation was achieved. The mixture was then stirred in a sealed flask at 50° C. for 24 hours. The reaction mixture was concentrated to leave a residue which was subjected to chromatography (SiO₂) eluting with CHCl₃/iPrOH (2%). Concentration of the desired fractions afforded the title compound as a white powder (0.195 g, 0.58 mmol, 38% over 3 steps). ¹H NMR (DMSO, 400 MHz) δ 8.20 (br s, 1H), 8.07 (d, 2H, J=10 Hz), 7.86 (br s, 2H), 7.54 (br s, 1H), 7.22 (br s, 2H). LCMS (M+H=339.09® 6.00 minutes).

### 5.2.3 2-[4-(3,4-dichlorophenyl)-2-methylamino-thieno[2,3-d]pyrimidine-6-yl]-methanol

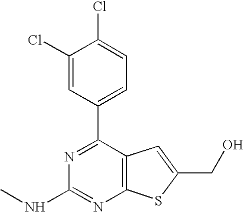

Step a: 2-[4-(3,4-dichlorophenyl)-2-methylsulphonyl-thieno[2,3-d]pyrimidine-6-carboxylic acid ethyl ester. 4-(3,4-Dichlorophenyl)-2-methylsulfanyl-thieno[2,3-d]pyrimidine-6-carboxylic acid ethyl ester (0.25 g, 0.63 mmol) was dissolved in CHCl₃ (10 mL). To this solution was added m-CPBA (2.2 equiv, 1.26 mmol, 0.28 g). The mixture was stirred for 1 hour. The mixture was then washed with NaHSO₃ (10% aq, 10 mL) and NaHCO₃ (10% aq, 10 mL). The chloroform solution was dried (Na₂SO₄), filtered, and concentrated.

Step b: 2-[4-(3,4-dichlorophenyl)-2-methylamino-thieno[2,3-d]pyrimidine-6-carboxylic acid ethyl ester. The crude sulfone from step (a) was dissolved in THF (5 mL) and MeNH₂ (2M in THF, 5 mL) was added. The mixture was stirred in a sealed tube at 70° C. for 12 hours. The mixture was cooled, concentrated, and then dissolved in toluene/CH₂Cl₂ (1:1 v/v, 5 mL).

Step c: 2-[4-(3,4-dichlorophenyl)-2-methylamino-thieno[2,3-d]pyrimidine-6-yl]-methanol To the solution from step (b) was added DIBAL (3.3 equiv, 2.1 mmol) in toluene (2.1 mL, 1M solution) at 0° C. The mixture was stirred 1 hour before being quenched with HCl (5% aq, 5 mL) and then extracted with NaOH (5% aq, 20 mL). The organic extracts were dried (Na₂SO₄), filtered, and concentrated. The crude product was subjected to chromatography (SiO₂, 20% EtOAc/hexanes). Concentration of the desired fractions afforded the title compound as a white solid (0.030 g, 17%). ¹H NMR (DMSO, 400 MHz) δ 8.06 (s, 1H), 7.84 (s, 2H), 7.40 (br s, 1H), 7.21 (s, 1H), 5.62 (t, 1H, J=5.6 Hz), 4.65 (dd, 2H, J₁=5.6 Hz, J₂=0.8 Hz), 2.89 (d, 3H, J=4.8 Hz). LCMS (M+H=340.1® 6.53 minutes).

### 5.2.4 2-[4-(3,4-dichlorophenyl)-2-methylamino-thieno[2,3-d]pyrimidine-6-yl]-propan-2-ol

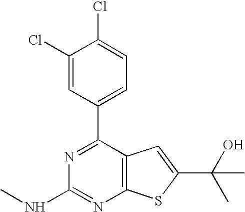

Step a: 2-[4-(3,4-dichlorophenyl)-2-methylsulfanyl-thieno[2,3-d]pyrimidine-6-yl]-propan-2-ol. 4-(3,4-Dichlorophenyl)-2-methylsulfanyl-thieno[2,3-d]pyrimidine-6-carboxylic acid ethyl ester (0.18 g, 0.45 mmol) was dissolved in THF (10 mL). To this solution was added MeMgBr (2.2 equiv, 1.0 mmol, 0.33 mL, 3M in diethyl ether) at 0° C. The mixture was allowed to come to room temperature over 2 hours. The reaction mixture was quenched with NH₄Cl (sat'd aq, 10 mL) and extracted with diethyl ether (3×10 mL). The organic extracts were dried (MgSO₄). After filtration, the ethereal extracts were concentrated to afford the crude dimethyl carbinol.

Step b: 2-[4-(3,4-dichlorophenyl)-2-methylsulfonyl-thieno[2,3-d]pyrimidine-6-yl]-propan-2-ol. The crude product from step (a) was dissolved in CHCl₃ (10 mL). To this solution was added m-CPBA (2.2 equiv, 1.0 mmol, 0.22 g). The mixture was stirred for 1 hour before being washed with NaHSO₃ (10% aq, 10 mL) and NaHCO₃ (10% aq, 10 mL). The chloroform solution was dried (Na₂SO₄), filtered, and concentrated.

Step c: 2-[4-(3,4-dichlorophenyl)-2-methylamino-thieno[2,3-d]pyrimidine-6-yl]-propan-2-ol. The crude sulfone from step (b) was dissolved in THF (5 mL). To this solution was added MeNH₂ (2M in THF, 5 mL). The mixture was stirred in a sealed tube at 70° C. for 12 hours. The reaction mixture was then cooled, concentrated, and subjected to preparatory HPLC for purification (30-80% CH₃CN/H₂O gradient over 30 minutes). The desired fractions were concentrated and then subjected to extraction with CHCl₃ (20 mL) and NaOH (5% aq, 20 mL). The chloroform solution was dried (Na₂SO₄), filtered, and concentrated to afford the title compound as a white solid (0.055 g, 33% over 3 steps). ¹H NMR (CDCl₃, 400 MHz) δ 7.94 (d, 1H, J=2.0 Hz), 7.66 (dd, 1H, J₁=8.4 Hz, J₂=2.0 Hz), 7.56 (d, 1H, J=8.4 Hz), 7.01 (s, 1H), 5.37 (q, 1H, J=4.8 Hz), 3.07 (d, 3H, J=4.8 Hz), 2.50 (br s, 1H), 1.67 (s, 6H). LCMS (M+H=368.1 @ 7.37 minutes).

### 5.2.5 5-Amino-4-(4-fluoro-3-methyl-phenyl)-2-methylamino-thieno[2,3-d]pyrimidine-6-carboxylic acid methylamide

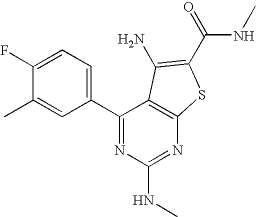

Step a: 4,6-Dichloro-2-methylsulfanyl-pyrimidine-5-carbaldehyde oxime. 4,6-Dichloro-2-methylsulfanyl-pyrimidine-5-carbaldehyde (9.3 g., 42 mmol; prepared according to 5.2.1, Step a) was suspended in 95% EtOH (100 mL) was warmed until a solution was obtained. Hydroxylamine hydrochloride (3.5 g, 105 mmol) dissolved in water (10 mL) was added to this solution followed by an addition of NaOH (2.5 g., 63 mmol) dissolved in water (25 mL). The reaction was stirred overnight. Concentration of the crude reaction mixture caused a precipitation, which was allowed to stand for 1 hr before being filtered off. The precipitate was then dried under high vacuum to yield the crude product (LC-MS: MH+ 238/240).

Step b: 4,6-Dichloro-2-methylsulfanyl-pyrimidine-5-carbonitrile. The crude 4,6-Dichloro-2-methylsulfanyl-pyrimidine-5-carboxaldehyde oxime (6 g., 33.6 mmol) from step (a) and POCl₃ (30 mL, 0.34 mmol) were mixed and warmed gradually to reflux. Heating was continued for 3 hours. After removal of excess POCl₃, the dense oil was poured into ice (with stirring). The solid obtained was purified using silica gel chromatography eluting with hexanes:EtOAc (9:1) to yield the desired material (3.5 g, 47% yield): ¹H-NMR (CDCl₃) δ 2.63 (s, 3H).

Step c: (6-Chloro-5-cyano-2-methylsulfanyl-pyrimidin-4-ylsulfanyl)-acetic acid ethyl ester. 4,6-Dichloro-2-methylsulfanyl-pyrimidine-5-carbonitrile (0.27 g., 1.2 mmol) from step (b) was dissolved in anhydrous THF (10 mL) was cooled to −5° C. Triethylamine (0.2 mL, 1.46 mmol) was added followed by a slow addition via syringe of a cold solution of Ethyl-2-mercapto-acetate (0.12 mL, 1.1 mmol) in THF (5 mL). After stirring at −5° C. for 30 minutes the reaction mixture was allowed to warm to room temperature. The solvents were removed in vacuo and the residue extracted with water and EtOAc. The combined organic layer was dried (MgSO₄) and concentrated to yield the desired product. (0.36 g, 97% yield) (LC-MS: MH+ 304).

Step d: 5-Amino-4-chloro-2-methylsulfanyl-thieno[2,3-d]pyrimidine-6-carboxylic acid ethyl ester. (6-Chloro-5-cyano-2-methylsulfanyl-pyrimidin-4-ylsulfanyl)-acetic acid ethyl ester, step (c), (0.36 g., 1.18 mmol) and di-isopropylethylamine (0.5 mL) in toluene (10 mL) were heated in a sealed tube at reflux overnight. The solvents were removed, and the solid obtained was sonicated with a small amount of ethyl acetate and filtered to yield the desired product (0.21 g, 60% yield) ¹H-NMR (CDCl₃) δ6.66 (br s, 2H), 4.35 (q, 2H), 2.63 (s, 3H), 1.38 (t, 3H); LC-MS: MH+=304.

Step e: 5-Amino-4-(4-fluoro-3-methyl-phenyl)-2-methylsulfanyl-thieno[2,3-d]pyrimidine-6-carboxylic acid ethyl ester. The title compound was prepared following standard Suzuki coupling conditions (as detailed in 5.2.1, Step d) from 5-Amino-4-chloro-2-methylsulfanyl-thieno[2,3-d]pyrimidine-6-carboxylic acid ethyl ester (0.215 g, 0.7 mmol), step (d) above, to yield the title compound (0.2 g, 75% yield) ¹H-NMR (CDCl₃) δ 7.49 (d, 1H), 7.43 (m, 1H), 7.18 (t, 1H), 5.84 (br s, 2H), 4.33 (q, 2H), 2.65 (s, 3H), 2.37 (d, 3H), 1.38 (t, 3H); LC-MS: MH+=378.

Step f: 5-Amino-4(4-fluoro-3-methyl-phenyl)-2-methylsulfanyl-thieno[2,3-d]pyrimidine-6-carboxylic acid. 5-Amino-4-(4-fluoro-3-methyl-phenyl)-2-methylsulfanyl-thieno[2,3-d]pyrimidine-6-carboxylic acid ethyl ester (0.2 g, 0.53 mmol) from step (e) was treated with LiOH (0.125 g, 53 mmol) in water:THF (1:1 v/v; 10 mL) and heated at reflux overnight. The excess THF was removed and the residue neutralized to pH 4 with dilute HOAc. The solid obtained is filtered and washed with water, dried under high vacuum to yield the title compound (0.163 g, 88% yield); LC-MS: MH+=350.

Step g: 5-Amino-4-(4-fluoro-3-methyl-phenyl)-2-methylsulfanyl-thieno[2,3-d]pyrimidine-6-carboxylic acid methylamide. The title compound was prepared from 5-Amino-4-(4-fluoro-3-methyl-phenyl)-2-methylsulfanyl-thieno[2,3-d]pyrimidine-6-carboxylic acid (0.16 g, 0.45 mmol) and Methylamine (2M in THF, 2 mL) using standard coupling conditions (HOBT/EDAC). The crude reaction mixture was treated with water and extracted with ethyl ether to yield the title compound (0.15 g, 90% yield); LC-MS: MH+=363.

Step h: 5-Amino-4-(4-fluoro-3-methyl-phenyl)-2-methylamino-thieno[2,3-d]pyrimidine-6-carboxylic acid methylamide. The title compound was prepared by treatment of 5-Amino-4(4-fluoro-3-methyl-phenyl)-2-methylsulfanyl-thieno[2,3-d]pyrimidine-6-carboxylic acid-methylamide (0.15 g., 0.4 mmol) with m-CPBA (0.12 g., 0.7 mmol) in dichloromethane at 0° C. for 1 hour. The reaction mixture was partitioned between saturated NaHSO₃ solution (5 mL) and dichloromethane. The organic layer was separated, concentrated to a small volume and treated with excess methylamine in THF (2M solution; 2 mL) for 2 hours. The solvent was removed and the crude product subjected to silica gel chromatography eluting with hexanes:EtOAc (3:1) to give the desired title compound (25 mg., 18% yield): 1H-NMR (CDCl3) δ: 7.43 (m, 2H), 7.16 (t, 1H), 5.92 (br s, 2H), 5.35 (br s, 1H), 5.28 (br s, 1H), 3.09 (d, 3H), 2.95 (d, 3H), 2.36 (s, 3H); LC-MS MH+=346; m.p.: 240-242° C. (dec.).

### 5.2.6 5-Amino-4-(3,4-dichlorophenyl)-2-methylamino-thieno[2,3-d]pyrimidine-6-carboxylic acid methylamide

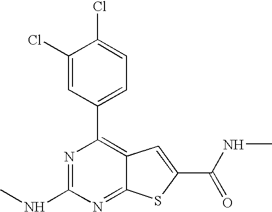

Step a: 5-cyano-4-(3,4-dichlorophenyl)-2-methylsulfanyl-pyrimidin-6-one A solution of 3,4-dichlorobenzaldehyde (4.34 g, 24.8 mmol), potassium carbonate (3.63 g, 26.3 mmol), ethyl cyanoacetate (2.80 mL, 26.3 mmol), S-methylisothiourea sulfate (3.45 g, 12.4 mmol) in EtOH (150 mL) were heated at 70° C. for 60 hours. The precipitate was filtered off, washed with EtOH and then dissolved in boiling water (500 mL). On cooling, the precipitate was isolated and dried by repeated addition and evaporation of toluene to yield the desired compound (3.90 g, 50%): ¹H-NMR (DMSO) δ 7.96 (m, 1H), 7.76 (m, 2H), 2.35 (s, 3H).

Step b: 6-chloro-5-cyano-4-(3,4-dichlorophenyl)-2-methylsulfanylpyrimidine The material (3.90 g, 12.5 mmol) from step (a) was suspended in POCl₃ (10 mL) and 1,4-dioxane (40 mL) and heated at reflux for 16 hours. On cooling the reaction mixture was poured into ice-water and adjusted to pH 7 by addition of solid potassium carbonate. The solution was extracted with EtOAc, the combined organic layers washed with brine, dried (Na₂SO₄) and evaporated to yield the desired compound (3.75 g, 90%): ¹H-NMR (DMSO) δ: 8.20 (m, 1H), 7.96 (m, 2H), 2.66 (s, 3H).

Step c: [6-(3,4-dichlorophenyl)-5-cyano-2-methylsulfanyl-pyrimidin-4-ylsulfanyl]-acetic acid ethyl ester. The product (3.72 g, 11.25 mmol) from step (b) was suspended in EtOH—CH₂Cl₂ (60 mL; 1:1 v/v) at 0° C., ethyl mercaptoacetate (1.23 mL, 11.25 mmol) was added followed by Hunigs base (2.10 mL, 12.0 mmol). The reaction mixture was stirred at that temperature for 15 minutes, then at ambient temperature for a further 15 minutes. The reaction mixture was partitioned between EtOAc: NH₄Cl (aq.), and the combined organic phases were washed with brine, dried (Na₂SO₄) and evaporated to yield the desired compound (3.85 g, 83%).

Step d: 5-Amino-4-(3,4-dichlorophenyl)-2-methylsulfanyl-thieno[2,3-d]pyrimidine-6-carboxylic acid ethyl ester. The material (3.85 g, 9.30 mmol) from step (c) was dissolved in toluene: EtOH (20 mL; 1:1 v/v), Hunigs base (4.0 mL, 22.9 mmol) added and the reaction mixture then heated at 100° C. in a sealed tube for 16 hours. The precipitate was filtered off, washed with EtOH to yield the desired product as a solid (3.58 g, 93%).

Step e: 5-Amino-4-(3,4-dichlorophenyl)-2-methylsulfanyl-thieno[2,3-d]pyrimidine-6-carboxylic acid. The material (3.57 g, 8.82 mmol) from step (d) was treated with LiOH (1.08 g, 25.7 mmol) in water:THF (1:2 v/v; 30 mL) and heated at reflux overnight. The reaction mixture was poured into water, this then adjusted to pH 2 with dilute HCl. The reaction mixture was extracted with EtOAc, the combined organic phases were washed with brine, dried (Na₂SO₄) and evaporated to yield the title compound (2.90 g, 88%): ¹H-NMR (DMSO) δ 7.95 (d, 1H), 7.75 (dd, 1H), 7.66 (dd, 1H), 6.16 (br s, 2H), 2.61 (s, 3H).

The material from step (e) was then carried through the reactions outlined in 5.2.5, Steps g-h to yield the desired material 5-Amino-4-(3,4-dichlorophenyl)-2-methylamino-thieno[2,3-d]pyrimidine-6-carboxylic acid methylamide: ¹H-NMR (CDCl₃) δ 7.96 (s, 1H), 7.74 (s, 1H), 7.70 (d, 1H, J=8.4 Hz), 7.60 (d, 1H, J=8.4 Hz), 7.02 (br s, 1H), 2.98 (d, 3H, J=5.2 Hz), 2.58 (s, 3H); MH+ 367; Calcd for C₁₅H₁₂Cl₂N₄OS.H₂O: C, 46.76; H, 3.66; N, 14.54. Found: C, 46.76; H, 3.14; N, 14.14.

### 5.2.7 5-Amino-2-methylamino-4-(3-pyrrolidin-1-yl-phenyl)-thieno[2,3-d]pyrimidine-6-carboxylic acid methyl amide

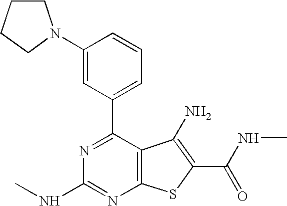

Aryl bromide (0.18 g, 0.46 mmol; prepared according to the reactions outlined in 5.2.6), pyrrolidine (3 equiv, 0.13 mL), Cs₂CO₃ (2 equiv, 0.33 g), Pd₂ dba₃ (5 mol %, 21 mg), and 4,5-bis(diphenylphosphino)-9,9-dimethylxanthene (XANT PHOS, 10 mol %, 27 mg) were combined together in dioxane (2 mL). The mixture was stirred under an inert atmosphere at 100° C. for 48 hours. The mixture was filtered and then concentrated. The crude residue was subjected to preparatory HPLC (20-70% CH₃CN/H₂O gradient over 30 minutes). The desired fractions were concentrated and then extracted with NaOH (5% aq, 20 mL) and CHCl₃ (20 mL). The chloroform layer was dried (Na₂SO₄), filtered, and concentrated to afford the title compound as a pale yellow powder (0.019 g, 11%). ¹H NMR (CDCl₃, 400 MHz) δ7.34 (t, 1H, J=7.6 Hz), 6.75 (d, 1H, J=7.6 Hz), 6.68 (dt, 1H, J1=8.4 Hz, J2=2.0 Hz), 6.63 (br s, 1H), 6.11 (br s, 2H), 5.48 (br s, 1H), 5.26 (q, 1H, J=4.8 Hz), 3.31 (t, 4H, J=6.4 Hz), 3.06 (d, 3H, J=5.2 Hz), 2.93 (d, 3H, J=5.2 Hz), 2.01 (m, 4H). LCMS (M+H=383.1 @ 6.56 minutes).

### 5.2.8 5-amino-4-(3,4-dichlorophenyl)-2-[(2-methylpropyl)amino]thiopheno[2,3-d]pyrimidine-6-carboxamide

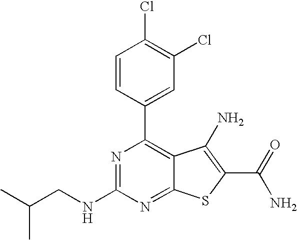

Step a: 6-(3,4-dichlorophenyl)-4-hydroxy-2-methylthiopyrimidine-5-carbonitrile. 3,4-dichlorobenzaldehyde (9.46 g, 54.0 mmol), ethylcyanoacetate (6.35 mL, 59.4 mmol), methylisothiourea sulfate (7.51 g, 27 mmol), potassium carbonate (8.20 g, 59.4 mmol) and anhydrous ethanol (300 mL) were placed in a round-bottomed flask equipped with a condenser. The reaction was heated to 80° C. for about three days. The reaction mixture was filtered and the precipitate was dried. The dried precipiate was suspended in water (100 mL) and filtered to give 12.1 g of crude material as a white solid. MS m/z calculated for (M+H)⁺ 312, found 312.

Step b: 6-(3,4-dichlorophenyl)-4-chloro-2-methylthiopyrimidine-5-carbonitrile. 6-(3,4-dichlorophenyl)-4-hydroxy-2-methylthiopyrimidine-5-carbonitrile was dissolved in anhydrous dioxane (80 mL), POCl₃ (20 mL) was added, and the mixture was heated at 105° C. overnight. Volatiles were removed in vacuo and the crude mixture was poured into ice water. The mixture was neutralized using solid potassium carbonate. Extraction was carried out with EtOAc (3×200 mL), the organic layers were combined and dried with sodium sulfate, and dried in vacuo to give 8.0 g as a yellow solid. ¹H NMR (400 MHz, D6-DMSO) δ ppm 2.62 (s, 3H) 7.92 (m, 2H) 8.11 (m, 2H).

Step c: 2-[6-(3,4-dichlorophenyl)-5-cyano-2-methylthiopyrimidin-4-ylthio]acetamide. Crude 6-(3,4-dichlorophenyl)-4-chloro-2-methylthiopyrimidine-5-carbonitrile (12.93 g, 39.10 mmol), Hunnigs base (10.2 mL, 58.70 mmol), 2-mercaptoacetamide (3.6 g, 39.10 mmol), dichloromethane (150 mL), and Ethanol (150 mL) were place in a round-bottomed flask at 0° C. for 30 minutes. The reaction was allowed to warm to room temperature and stirred for 1 hour. The precipitate was filtered to give 12.0 g as a white solid. MS m/z calculated for (M+H)⁺ 385, found 385. ¹H NMR (400 MHz, D6-DMSO) δ ppm 2.63 (s, 3H) 4.08 (s, 2H), 7.31 (s, 1H) 7.71 (s, 1H) 7.90 (m, 2H) 8.15 (m, 1H).

Step d: 5-amino-4-(3,4-dichlorophenyl)-2-methylthiothiopheno[2,3-d]pyrimidine-6-carboxamide. Crude 2-[6-(3,4-dichlorophenyl)-5-cyano-2-methylthiopyrimidin-4-ylthio]acetamide (11.65 g, 30.25 mmol) was placed in a freshly prepared solution of NaOEt/HOEt (1.4 g Na, 60.50 mmol, 250 ml anhydrous ethanol) and stirred at room temperature for 1 hour. The precipitate was filtered to give 9.8 g as a light yellow solid. MS m/z calculated for (M+H)⁺ 385, found 385. ¹H NMR (400 MHz, D6-DMSO) δ ppm 2.61 (s, 3H) 6.30 (s, 2H), 7.34 (s, 2H) 7.66 (dd, J=2, 8 Hz, 1H) 7.83 (d, J=2 Hz, 1H) 7.93 (d, J=2 Hz, 1H).

Step e: 5-amino-4-(3,4-dichlorophenyl)-2-(methylsulfinyl)thiopheno[2,3-d]pyrimidine-6-carboxamide. 5-amino-4-(3,4-dichlorophenyl)-2-methylthiothiopheno[2,3-d]pyrimidine-6-carboxamide (1.5 g, 3.89 mmol) was dissolved in chloroform (75 mL), meta-chloroperbenzoic acid (1.48 g, 8.56 mmol) was added and the mixture was stirred at room temperature for 2 hours. The reaction was quenched with 10% aqueous HSO₃ (75 mL) and 10% aqueous NaHCO₃ (75 mL). Layers were separated, extracted with chloroform (3×100 mL), dried with MgSO₄, and concentrated in vacuo to give 1.3 g as an orange solid. MS m/z calculated for (M+H)⁺ 401, found 401.

Step f: 5-amino-4-(3,4-dichlorophenyl)-2-[(2-methylpropyl)amino]thiopheno[2,3-d]pyrimidine-6-carboxamide. 5-amino-4-(3,4-dichlorophenyl)-2-(methylsulfinyl)thiopheno[2,3-d]pyrimidine-6-carboxamide (200 mg, 0.50 mmol), iso-butylamine (0.50 mL, 5.0 mmol), and anhydrous THF (6 mL) were placed in a round-bottomed flask and heated at 70° C. overnight. The volatiles were removed in vacuo, the residue was suspended in ethanol. The precipitate was collected using vacuum filtration to give 60 mg (29%) as a yellow solid. ¹H NMR (300 MHz, D6-DMSO) δ ppm 0.90 (d, J=6 Hz, 6H) 1.87 (m, 1H) 3.16 (t, J=6 Hz, 2H) 6.13 (s, 2H) 7.00 (s, 2H) 7.56 (dd, J=2, 8 Hz, 1H) 7.79 (d, J=8 Hz, 1H) 7.85 (d, J=2 Hz, 1H). MS m/z calculated for (M+H)⁺ 410, found 410. Analytical HPLC retention time: 20.53 minutes (5-70% MeCN/water 20 minutes, ODS).

### 5.2.9 5-amino-4-(3, 4-dichlorophenyl)-2-[(methylpropyl)amino]thiopheno[2,3-d]pyrimidine-6-carboxamide

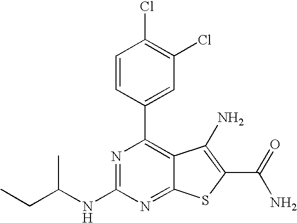

5-amino-4-(3,4-dichlorophenyl)-2-(methylsulfinyl)thiopheno[2,3-d]pyrimidine-6-carboxamide (200 mg, 0.50 mmol), sec-butylamine (0.50 mL, 5.0 mmol), and anhydrous THF (6 mL) were placed in a round-bottomed flask and heated at 70° C. overnight. The volatiles were removed in vacuo, the residue suspended in Ethanol, and the precipitate was collected using vacuum filtration to give 45 mg (22%) as a yellow solid. ¹H NMR (300 MHz, D6-DMSO) δ ppm 0.88 (t, J=6 Hz, 3H) 1.13 (d, J=9 Hz, 3H) 1.51 (m, 2H) 3.94 (m, 1H) 6.13 (s, 2H) 7.00 (s, 2H) 7.58 (dd, J=2, 8 Hz, 1H) 7.79 (d, J=8 Hz, 1H) 7.86 (d, J=2 Hz, 1H). MS m/z calculated for (M+H)⁺ 410, found 410. Analytical HPLC retention time: 20.56 minutes (5-70% MeCN/water 20 minutes, ODS).

### 5.2.10 5-amino-2-azetidinyl-4-(3,4-dichlorophenyl)thiopheno[2,3-d]pyrimidine-6-carboxamide

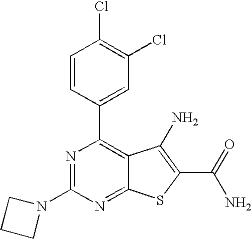

5-amino-4-(3,4-dichlorophenyl)-2-(methylsulfinyl)thiopheno[2,3-d]pyrimidine-6-carboxamide (60 mg, 0.15 mmol), azetidine (0.100 mg, 1.75 mmol), and anhydrous THF (4 mL) were placed in a round-bottomed flask and heated at 70° C. overnight. The volatiles were removed in vacuo, the residue suspended in Ethanol, and the precipitate was collected using vacuum filtration to give 10 mg (17 as a yellow solid. ¹H NMR (300 MHz, D6-DMSO) δ ppm 2.34 (m, 2H) 4.13 (m, 4H) 6.16 (s, 2H) 7.05 (s, 2H) 7.57 (dd, J=2, 8 Hz, 1H) 7.79 (d, J=8 Hz, 1H) 7.84 (d, J=2 Hz, 1H). MS m/z calculated for (M+H)⁺ 394, found 394. Analytical HPLC retention time: 10.60 min (20-100% MeCN/water 20 minutes, ODS).

### 5.2.11 5-amino-4-(3,4-dichlorophenyl)-2-pyrrolidinylthiopheno[2,3-d]pyrimidine-6-carboxamide

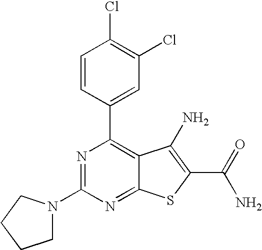

5-amino-4-(3,4-dichlorophenyl)-2-(methylsulfinyl)thiopheno[2,3-d]pyrimidine-6-carboxamide (187 mg, 0.46 mmol), pyrrolidine (0.38 mL, 4.65 mmol), and anhydrous THF (6 mL) were placed in a round-bottomed flask and heated at 70° C. overnight. The volatiles were removed in vacuo, the residue suspended in Ethanol, and the precipitate was collected using vacuum filtration to give 58 mg (31%) as a yellow solid. ¹H NMR (300 MHz, D6-DMSO) δ ppm 1.94 (m, 4H) 3.56 (m, 4H) 6.18 (s, 2H) 7.02 (s, 2H) 7.59 (dd, J=2, 8 Hz, 1H) 7.79 (d, J=8 Hz, 1H) 7.86 (d, J=2 Hz, 1H). MS m/z calculated for (M+H)⁺ 408, found 408. Analytical HPLC retention time: 11.58 minutes (20-100% MeCN/water 20 minutes, ODS).

### 5.2.12 5-amino-4-(3,4-dichlorophenyl)-2-[(3-hydroxypropyl)amino]thiopheno[2,3-d]pyrimidine-6-carboxamide

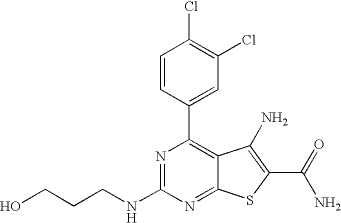

5-amino-4-(3,4-dichlorophenyl)-2-(methylsulfinyl)thiopheno[2,3-d]pyrimidine-6-carboxamide (100 mg, 0.25 mmol), 3-aminopropanol (0.06 mL, 0.78 mmol), triethylamine (0.20 mL, 1.43 mmol), and anhydrous THF (2 mL) were placed in a microwave tube and heated at 130° C. for 10 minutes. The volatiles were removed in vacuo, the residue suspended in Ethanol, and the precipitate was collected using vacuum filtration to give 39 mg (38%) as a yellow solid. ¹H NMR (400 MHz, D6-DMSO) δ ppm 1.71 (m, 2H) 3.38 (m, 2H) 3.48 (m, 2H) 4.48 (bs, 1H) 6.13 (s, 2H) 7.01 (s, 2H) 7.58 (dd, J=2, 8 Hz, 1H) 7.79 (d, J=8 Hz, 1H) 7.86 (d, J=2 Hz, 1H). MS m/z calculated for (M+H)⁺ 412, found 412. Analytical HPLC retention time: 4.91 minutes

### 5.2.13 5-amino-4-(3,4-dichlorophenyl)-2-[(4-hydroxybutyl)amino]thiopheno[2,3-d]pyrimidine-6-carboxamide

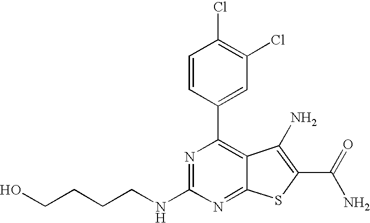

5-amino-4-(3,4-dichlorophenyl)-2-(methylsulfinyl)thiopheno[2,3-d]pyrimidine-6-carboxamide (200 mg, 0.50 mmol), 4-aminobutanol (0.10 mL, 1.08 mmol), triethylamine (0.20 mL, 1.43 mmol), and anhydrous THF (2 mL) were placed in a microwave tube and heated at 130° C. for 10 minutes. The volatiles were removed in vacuo, the residue suspended in Ethanol, and the precipitate was collected using vacuum filtration to give 105 mg (49%) as a yellow solid. ¹H NMR (400 MHz, D6-DMSO) δ ppm 1.47 (m, 2H) 1.58 (m, 2H) 3.34 (m, 2H) 3.41 (m, 2H) 4.38 (bs, 1H) 6.13 (s, 2H) 6.99 (s, 2H) 7.58 (dd, J=2, 8 Hz, 1H) 7.79 (d, J=8 Hz, 1H) 7.85 (d, J=2 Hz, 1H). MS m/z calculated for (M+H)⁺ 426, found 426. Analytical HPLC retention time: 6.05 minutes

### 5.2.14 5-amino-4-(3,4-dichlorophenyl)-2-[(methylethyl)amino]thiopheno[2,3-d]pyrimidine-6-carboxamide

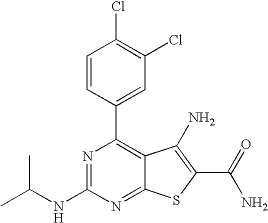

5-amino-4-(3,4-dichlorophenyl)-2-(methylsulfinyl)thiopheno[2,3-d]pyrimidine-6-carboxamide (150 mg, 0.37 mmol), isopropylamine (0.16 mL, 1.86 mmol), and anhydrous THF (2 mL) were placed in a microwave tube and heated at 130° C. for 10 minutes. The volatiles were removed in vacuo, the residue suspended in Ethanol, and the precipitate was collected using vacuum filtration to give 82 mg (56%) as a yellow solid. ¹H NMR (400 MHz, D6-DMSO) δ ppm 1.17 (d, J=4 Hz, 6H) 4.1 (m, 1H) 6.14 (s, 2H) 6.99 (s, 2H) 7.56 (dd, J=2, 8 Hz, 1H) 7.79 (d, J=8 Hz, 1H) 7.86 (d, J=2 Hz, 1H). MS m/z calculated for (M+H)⁺ 396, found 396. Analytical HPLC retention time: 6.16 minutes

### 5.2.15 5-amino-4-(3,4-dichlorophenyl)-2-[(2-hydroxyethyl)amino]thiopheno[2,3-d]pyrimidine-6-carboxamide

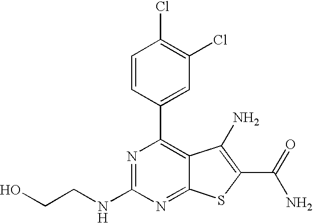

5-amino-4-(3,4-dichlorophenyl)-2-(methylsulfinyl)thiopheno[2,3-d]pyrimidine-6-carboxamide (200 mg, 0.25 mmol), 2-aminoethanol (0.1 mL, 1.65 mmol), triethylamine (0.20 mL, 1.43 mmol), and anhydrous THF (2 mL) were placed in a microwave tube and heated at 130° C. for 10 minutes. The volatiles were removed in vacuo, the residue suspended in Ethanol, and the precipitate was collected using vacuum filtration to give 90 mg (90%) as a yellow solid. ¹H NMR (400 MHz, D6-DMSO) δ ppm 1.76 (bs, 1H) 3.41 (t, J=4 Hz, 2H) 3.54 (t, J=4 Hz, 2H) 4.69 (s, 1H), 6.14 (s, 2H) 7.01 (s, 2H) 7.57 (dd, J=2, 8 Hz, 1H) 7.79 (d, J=8 Hz, 1H) 7.85 (d, J=2 Hz, 1H). MS m/z calculated for (M+H)⁺ 398, found 398. Analytical HPLC retention time: 4.79 minutes

### 5.2.16 5-amino-4-(3,4-dichlorophenyl)-2-(methylethoxy)thiopheno[2,3-d]pyrimidine-6-carboxamide

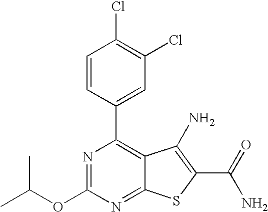

60% NaH (0.42 g, 16.6 mmol) was placed in anhydrous isopropanol and allowed to react. 5-amino-4-(3,4-dichlorophenyl)-2-(methylsulfinyl)thiopheno[2,3-d]pyrimidine-6-carboxamide (710 mg, 1.77 mmol) was added and allowed to stir at room temperature for 2 hours. The reaction was poured into a mixture of 1.0N HCl (20 mL) and water (150 mL), and the precipitate was filtered. The reaction was purified using Prep (20-100 MeCN, water), and volatiles were removed in vacuo. The reaction was dissolved in EtOAc, washed with sat. NaHCO₃ (50 mL) and the organic layer was dried with Na₂SO₄. Volatiles were removed in vacuo to give 150 mg (21%) as a yellow solid. ¹H NMR (300 MHz, D6-DMSO) δ ppm 1.36 (d, J=6 Hz, 6H) 5.3 (m, 1H) 6.30 (s, 2H) 7.27 (s, 2H) 7.65 (dd, J=3.9 Hz, 1H) 7.83 (d, J=9 Hz, 1H) 7.93 (d, J=2 Hz, 1H). MS m/z calculated for (M+H)⁺ 397, found 397. Analytical HPLC retention time: 6.20 minutes

### 5.2.17 5-amino-4-(3,4-dichlorophenyl)-2-ethoxythiopheno[2,3-d]pyrimidine-6-carboxamide

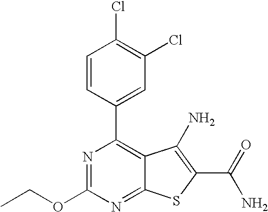

Sodium metal (35 mg, 1.55 mmol) was placed in anhydrous ethanol and allowed to react. 5-amino-4-(3,4-dichlorophenyl)-2-(methylsulfinyl)thiopheno[2,3-d]pyrimidine-6-carboxamide (125 mg, 0.31 mmol) was added and allowed to stir at room temperature for 1 hour. The reaction was poured into a mixture of 1.0N HCl (20 mL) and water (150 mL), and the precipitate was filtered. The reaction was purified using Prep (20-100 MeCN, water), and volatiles were removed in vacuo. The reaction was dissolved in EtOAc, washed with sat. NaHCO₃ (50 mL) and the organic layer was dried with Na₂SO₄. Volatiles were removed in vacuo to give 15 mg (13%) as a yellow solid. ¹H NMR (300 MHz, D6-DMSO) δ ppm 1.37 (t, J=6 Hz, 3H) 4.45 (q, J=6 Hz, 2H) 6.31 (s, 2H) 7.27 (s, 2H) 7.65 (dd, J=3, 9 Hz, 1H) 7.83 (d, J=9 Hz, 1H) 7.93 (d, J=2 Hz, 1H). MS m/z calculated for (M+H)⁺ 383, found 383. Analytical HPLC retention time: 6.01 minutes

### 5.2.18 5-amino-4-(3,4-dichlorophenyl)-2-cyanothiopheno[2,3-d]pyrimidine-6-carboxamide

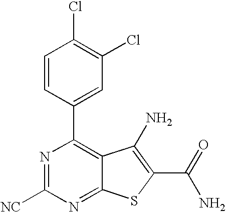

5-amino-4-(3,4-dichlorophenyl)-2-(methylsulfinyl)thiopheno[2,3-d]pyrimidine-6-carboxamide (500 mg, 1.25 mmol) and sodium cyanide (92 mg, 1.88 mmol) were dissolved in anhydrous DMF (10 mL) at 0° C. and allowed to stir for 2 hours. The reaction mixture was poured into water and extracted with EtOAc (3×50 mL). The combined organic layers were dried with sodium sulfate and volatiles were removed in vacuo. The reaction was purified using preparative HPLC (20-100% MeCN/H₂O 30 minutes) to give 75 mg (17%) as a yellow solid. ¹H NMR (400 MHz, D6-DMSO) δ ppm 6.44 (s, 2H) 7.39 (s, 2H) 7.67 (dd, J=2, 8 Hz, 1H) 7.82 (d, J=8 Hz, 1H) 7.93 (d, J=2 Hz, 1H). MS m/z calculated for (M+H)⁺ 364, found 364. Analytical HPLC retention time: 6.05 minutes

### 5.2.19 5-amino-4-(3,4-dichlorophenyl)-2-(cyclopropylmethyl)thiopheno[2,3-d]pyrimidine-6-carboxamide

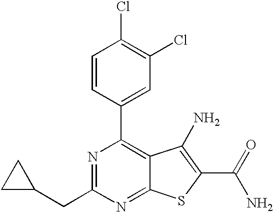

Step a: 6-(3,4-dichlorophenyl)-2-(cyclopropylmethyl)-4-hydroxypyrimidine-5-carbonitrile. 3,4-Dichlorobenzaldehyde (2.0 g, 11.42 mmol), ethyl-cyanoacetate (1.3 mL, 12.56 mmol), 2-cyclopropylethanamidine (1.7 g, 12.56 mmol), potassium carbonate (1.57 g, 13.70 mmol), and EtOH (40 mL) were placed in a round-bottomed flask and heated at 80° C. for 14 hours. Solids were filtered, volatiles were removed in vacuo, and the resulting residue was dissolved residue in ethyl acetate (100 mL) and washed with water (2×100 mL). The combined organic layers were dried with sodium sulfate and concentrated in vacuo to give 2.1 g as a yellow solid. MS m/z calculated for (M+H)⁺ 320, found 320.

Step b: 6-(3,4-dichlorophenyl)-4-chloro-2-(cyclopropylmethyl)pyrimidine-5-carbonitrile. 6-(3,4-dichlorophenyl)-2-(cyclopropylmethyl)-4-hydroxypyrimidine-5-carbonitrile (2.1 g, 6.6 mmol) was dissolved in anhydrous dioxane (20 mL) and POCl₃ (5 mL) was added via syringe. The reaction was heated at 100° C. overnight. The reaction was quenched with ice water and neutralized with potassium carbonate. The aqueous layer was extracted with ethyl acetate (3×100 mL), the combined organic layers were dried with sodium sulfate, and concentrated in vacuo to give 1.66 g as a light brown solid. MS m/z calculated for (M+H)⁺ 338, found 338.

Step c: 2-[6-(3,4-dichlorophenyl)-5-cyano-2-(cyclopropylmethyl)pyrimidin-4-ylthio]acetamide. 6-(3,4-dichlorophenyl)-4-chloro-2-(cyclopropylmethyl)pyrimidine-5-carbonitrile (1.66 g, 4.90 mmol) was dissolved in dichloromethane (10 mL) and ethanol (10 mL). 2-mercaptoacetamide (490 mg, 5.39 mmol) was added and stirred at room temp for 1 hour. The precipitate was collected and washed with cold ethanol to give 1.4 g as a white solid. MS m/z calculated for (M+H)⁺ 393, found 393.

Step d: 5-amino-4-(3,4-dichlorophenyl)-2-(cyclopropylmethyl)thiopheno[2,3-d]pyrimidine-6-carboxamide. 2-[6-(3,4-dichlorophenyl)-5-cyano-2-(cyclopropylmethyl)pyrimidin-4-ylthio]acetamide (500 mg, 1.27 mmol) was added to a solution of NaOEt/EtOH (87 mg Na, 3.81 mmol, 5 mL EtOH) and allowed to stir at room temperature for 3 hours. The precipitate was collected and washed with cold EtOH (50 mL). The reaction was purified using preparative HPLC (20-100% MeCN/H₂O 30 minutes) to give 210 mg (43%) as a yellow solid. ¹H NMR (400 MHz, D6-DMSO) δ ppm 0.27 (m, 2H) 0.49 (m, 2H) 1.25 (m, 1H) 2.88 (d, J=7 Hz, 2H) 6.32 (s, 2H) 7.38 (s, 2H) 7.67 (dd, J=2, 8 Hz, 1H) 7.82 (d, J=8 Hz, 1H) 7.93 (d, J=2 Hz, 1H). MS m/z calculated for (M+H)⁺ 393, found 393. Analytical HPLC retention time: 6.34 minutes

### 5.2.20 5-amino-2-(cyclopropylamino)-4-(3-hydroxyphenyl)thiopheno[2,3-d]pyrimidine-6-carboxamide

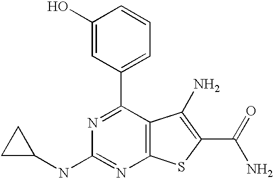

Step a: N-Cyclopropylguanidine hydrochloride. 2-methyl-2-thiopseudourea sulfate (20 g, 72 mmol), cyclopropyl amine (15 mL, 216 mmol), and water (40 mL) were heated together with stirring at 45° C. for 16 hours. The reaction mixture was cooled and concentrated in vacuo. Water was added (100 mL) and amberlite IRA 400 (Cl⁻) resin was added. The mixture was stirred for 1 hour and then the resin was removed by filtration. The solution was concentrated in vacuo and azeotroped with methanol. The residue was crystallized from methanol-acetone to give 15 g (86%) of N-Cyclopropylguanidine hydrochloride as a white solid. ¹H NMR (400 MHz, D₂O): δ ppm 2.34-2.39 (m, 1H) 0.65-0.70 (m, 2H) 0.47-0.52 (m, 2H).

Step b: 2-(cyclopropylamino)-4-hydroxy-6-(3-methoxyphenyl)pyrimidine-5-carbonitrile. m-anisaldehyde (1.0 g, 7.3 mmol), ethylcyanoacetate (1.2 g, 12.0 mmol), potassium carbonate (2.0 g, 14.6 mmol), in EtOH (20 mL) were heated together with stirring at 60° C. for 10 minutes, followed by addition of N-cyclopropylguanidine hydrochloride (1.0 g, 12.0 mmol). The reaction mixture was heated with stirring at 60° C. for 16 h. The reaction mixture was cooled and the resulting precipitate was filtered off, washed with EtOH and dissolved in hot water. The solution was cooled to room temperature, acidified with 2N HCl to pH 2 and cooled to 0° C. in an ice bath. The resulting precipitate was filtered off, washed with ice water and dried by coevaporation with EtOH to give 1.63 g (80%) of 2-(cyclopropylamino)-4-hydroxy-6-(3-methoxyphenyl)pyrimidine-5-carbonitrile as a yellow solid. MS m/z calculated for (M+H)⁺ 283, found 283.

Step c: 4-chloro-2-(cyclopropylamino)-6-(3-methoxyphenyl)pyrimidine-5-carbonitrile. POCl₃ (10 mL) was added to a stirred solution of 2-(cyclopropylamino)-4-hydroxy-6-(3-methoxyphenyl)pyrimidine-5-carbonitrile (1.6 g, 5.6 mmol) in dry 1,4-dioxane (30 mL) and heated together with stirring at 90° C. for 3 hours. The mixture was cooled to room temperature and concentrated under reduced pressure. The residue was dissolved in 1,4-dioxane (5 mL) and ice water (50 mL) was added. The resulting precipitate was filtered off and dried by coevaporation with EtOH to give 0.9 g (60%) of 4-chloro-2-(cyclopropylamino)-6-(3-methoxyphenyl)pyrimidine-5-carbonitrile as a yellow solid. MS m/z calculated for (M+H)⁺ 301, found 301.

Step d: 5-amino-2-(cyclopropylamino)-4-(3-methoxyphenyl)thiopheno[2,3-d]pyrimidine-6-carboxamide. 4-chloro-2-(cyclopropylamino)-6-(3-methoxyphenyl)pyrimidine-5-carbonitrile (0.7 g, 2.3 mmol), mercaptoacetamide (0.3 g, 3.0 mmol), potassium carbonate (2.0 g, 14.6 mmol), in EtOH (20 mL) were heated together with stirring at 70° C. for 16 hours. The reaction mixture was cooled and 1.0M NaOC₂H₅ (10 mL) was added and heated at 70° C. for 1 hour. The mixture was cooled to room temperature and concentrated under reduced pressure and followed by dilution with EtOAc (100 mL). The mixture was washed with water (100 mL) and brine, dried over Na₂SO₄, filtered, and concentrated under reduced pressure. The residue was purified using HPLC (20-80% acetonitrile/water) to give 0.36 g (43%) of 5-amino-2-(cyclopropylamino)-4-(3-methoxyphenyl)thiopheno[2,3-d]pyrimidine-6-carboxamide as a yellow solid. ¹H NMR (400 MHz, DMSO-d₆): δ ppm 0.50-0.54 (m, 2H) 0.70-0.72 (m, 2H) 2.80-2.83 (m, 1H) 3.8 (s, 3H) 6.00 (s, 2H) 6.99 (s, 2H) 7.11-7.13 (m, 3H) 7.44-7.48 (m, 1H) 7.96 (s, 1H). MS m/z calculated for (M+H)⁺ 356, found 356. Analytical HPLC retention time: 4.96 minutes (0%-75% Acetonitrile/H₂O with 1% formic acid over 7 minutes; referred to herein as “method I”).

Step e: 5-amino-2-(cyclopropylamino)-4-(3-hydroxyphenyl)thiopheno[2,3-d]pyrimidine-6-carboxamide. Phosphorus tribromide (1.5 mL of 1.0M in dichloromethane, 1.56 mmol) was added via syringe over a period of 10 minutes to a solution of 5-amino-2-(cyclopropylamino)-4-(3-methoxyphenyl)thiopheno[2,3-d]pyrimidine-6-carboxamide (0.26 g, 0.73 mmol) in dry dichloromethane (20 mL) under nitrogen with stirring at 0° C. The reaction mixture was allowed to warm slowly and stirred at room temperature for 2 hours. The mixture was concentrated under reduced pressure and purified using HPLC (20-80% acetonitrile/water) to give 0.15 g (62%) of 5-amino-2-(cyclopropylamino)-4-(3-hydroxyphenyl)thiopheno[2,3-d]pyrimidine-6-carboxamide as a yellow solid. ¹H NMR (400 MHz, DMSO-d₆): δ ppm 0.50-0.54 (m, 2H) 0.70-0.72 (m, 2H) 2.80-2.83 (m, 1H) 6.00 (s, 2H) 6.91-6.99 (m, 5H) 7.31-7.35 (m, 1H) 7.93 (s, 1H) 9.98 (sb, 1H). MS m/z calculated for (M+H)⁺ 342, found 342. Analytical HPLC retention time: 4.32 minutes.

### 5.2.21 5-Amino-4-(2-chlorophenyl)-2-cyclopropylamino-thieno[2,3-d]pyrimidine-6-carboxamide

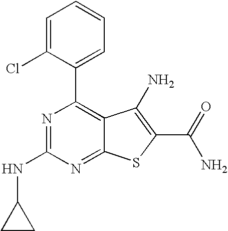

Step a: 5-Cyano-4-(2-chlorophenyl)-2-cyclopropylamino-6-oxopyrimidine. 2-Chlorobenzaldehyde (2.08 g, 14.8 mmol), ethylcyanoacetate (1.67 g, 14.8 mmol), N-cyclopropylguanidine.HCl (2.00 g, 14.8 mmol), and potassium carbonate (2.25 g, 16.3 mmol) were stirred in ethanol (70 mL) at 75° C. for 18 hours. The reaction mixture was partitioned between ethyl acetate and water. The organic layer was separated and concentrated to give a residue which was purified using flash chromatography (100% EtOAc) to give 5-cyano-4-(2-chlorophenyl)-2-cyclopropylamino-6-oxopyrimidine (2.33 g, 55%). MS m/z calculated for (M+H)⁺ 287, found 287.

Step b: 6-Chloro-5-cyano-4-(2-chlorophenyl)-2-cyclopropylaminopyrimidine. 5-Cyano-4-(2-chlorophenyl)-2-cyclopropylamino-6-oxopyrimidine (1.50 g, 5.24 mmol) was stirred in dioxane (25 mL) along with POCl₃ (12 mL). The mixture was heated at 90° C. for 2 hours and volatiles were then concentrated off. The residue was then partitioned between ethyl acetate and sat. NaHCO₃. The organic layer was separated and concentrated to give a residue which was recrystallized from ethanol to give 6-chloro-5-cyano-4-(2-chlorophenyl)-2-cyclopropylaminopyrimidine (0.60 g, 38%) as a yellow solid. MS m/z calculated for (M+H)⁺ 305, found 305.

Step c: 5-Amino-4-(2-chlorophenyl)-2-cyclopropylamino-thieno[2,3-d]pyrimidine-6-carboxamide. 6-Chloro-5-cyano-4-(2-chlorophenyl)-2-cyclopropylaminopyrimidine (0.60 g, 1.97 mmol), 2-mercaptoacetamide (0.18 g, 1.97 mmol), and sodium carbonate (0.21 g, 1.97 mmol) were stirred in ethanol (10 mL) at 60° C. for 4 hours. The reaction mixture was partitioned between ethyl acetate and water. The organic layer was separated and concentrated to give a residue. Excess sodium ethoxide in ethanol was added and the mixture was heated at 75° C. for 2 hours. The reaction mixture was partitioned between ethyl acetate and water. The organic layer was separated and concentrated to give a residue which was purified using preparative HPLC to give the title compound 5-amino-4-(2-chlorophenyl)-2-cyclopropylamino-thieno[2,3-d]pyrimidine-6-carboxamide (0.21 g, 29%) as a yellow solid. NMR (DMSO-d₆): δ 8.12 (s, 1H), 7.52-7.66 (m, 4H), 7.02 (s, 2H), 5.64 (s, 2H), 2.80 (m, 1H), 0.70 (t, 2H), 0.51 (t, 2H). MS m/z calculated for (M+H)⁺ 360, found 360.

### 5.2.22 5-Amino-4-(2-methoxyphenyl)-2-cyclopropylamino-thieno[2,3-d]pyrimidine-6-carboxamide

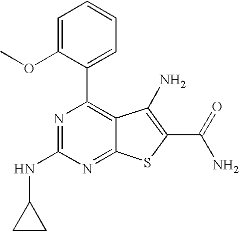

Step a: 5-Cyano-4-(2-methoxyphenyl)-2-cyclopropylamino-6-oxopyrimidine. 2-Methoxybenzaldehyde (1.51 g, 11.1 mmol), ethylcyanoacetate (1.25 g, 11.11 mmol), N-cyclopropylguanidine.HCl (1.50 g, 11.1 mmol), and potassium carbonate (1.69 g, 12.2 mmol) were stirred in ethanol (50 mL) at 75° C. for 18 hours. The reaction mixture was partitioned between ethyl acetate and water. The organic layer was separated and concentrated to give a residue which was purified using flash chromatography (100% EtOAc) to give 5-cyano-4-(2-methoxyphenyl)-2-cyclopropylamino-6-oxopyrimidine (1.31 g, 42%). MS m/z calculated for (M+H)⁺ 283, found 283.

Step b: 6-Chloro-5-cyano-4-(2-methoxyphenyl)-2-cyclopropylaminopyrimidine. 5-Cyano-4-(2-chlorophenyl)-2-cyclopropylamino-6-oxopyrimidine (0.80 g, 2.84 mmol) was stirred in POCl₃ (5 mL). The mixture was heated at 90° C. for 2 hours. then the volatiles were concentrated off and the residue was partitioned between ethyl acetate and sat. NaHCO₃. The organic layer was separated and concentrated to give a residue which was recrystallized from ethanol to give 6-chloro-5-cyano-4-(2-methoxyphenyl)-2-cyclopropylaminopyrimidine (0.53 g, 62%) as a yellow solid. MS m/z calculated for (M+H)⁺ 301, found 301.

Step c: 5-Amino-4-(2-methoxyphenyl)-2-cyclopropylamino-thieno[2,3-d]pyrimidine-6-carboxamide. 6-Chloro-5-cyano-4-(2-methoxyphenyl)-2-cyclopropylaminopyrimidine (0.30 g, 1.00 mmol), 2-mercaptoacetamide (0.09 g, 1.00 mmol), and sodium carbonate (0.11 g, 1.00 mmol) were stirred in ethanol (8 mL) at 60° C. for 4 hours. The reaction mixture was partitioned between ethyl acetate and water. The organic layer was separated and concentrated to give a residue. Excess sodium ethoxide in ethanol was added and the mixture was heated at 75° C. for 2 hours. The reaction mixture was partitioned between ethyl acetate and water. The organic layer was separated and concentrated to give a residue which was purified using preparative HPLC to give the title compound 5-amino-4-(2-methoxyphenyl)-2-cyclopropylamino-thieno[2,3-d]pyrimidine-6-carboxamide (0.07 g, 20%) as a yellow solid. NMR (DMSO-d₆): δ 7.95 (s, 1H), 7.11-7.60 (m, 4H), 6.95 (s, 2H), 5.71 (s, 2H), 3.72 (s, 3H), 2.79 (m, 1H), 0.70 (t, 2H), 0.50 (t, 2H). MS m/z calculated for (M+H)⁺ 356, found 356.

### 5.2.23 5-Amino-4-(3-cyanophenyl)-2-cyclopropylamino-thieno[2,3-d]pyrimidine-6-carboxamide

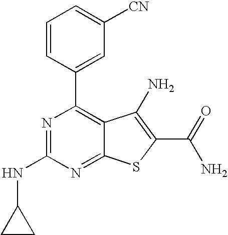

Step a: 5-Cyano-4-(3-cyanophenyl)-2-cyclopropylamino-6-oxopyrimidine. 3-Cyanobenzaldehyde (1.94 g, 14.8 mmol), ethylcyanoacetate (1.67 g, 14.8 mmol), N-cyclopropylguanidine.HCl (2.00 g, 14.8 mmol), and potassium carbonate (2.25 g, 16.3 mmol) were stirred in ethanol (70 mL) at 75° C. for 18 hours. The reaction mixture was partitioned between ethyl acetate and water. The organic layer was separated and concentrated to give a residue which was purified using flash chromatography (100% EtOAc) to give 5-cyano-4-(3-cyanophenyl)-2-cyclopropylamino-6-oxopyrimidine (0.52 g, 12%). MS m/z calculated for (M+H)⁺ 278, found 278.

Step b: 6-Chloro-5-cyano-4-(3-cyanophenyl)-2-cyclopropylaminopyrimidine. 5-Cyano-4-(3-cyanophenyl)-2-cyclopropylamino-6-oxopyrimidine (0.52 g, 1.88 mmol) was stirred in POCl₃ (5 mL). The mixture was heated at 90° C. for 2 hours. and then volatiles were concentrated off and the residue was partitioned between ethyl acetate and sat. NaHCO₃. The organic layer was separated and concentrated to give a residue which was recrystallized from ethanol to give 6-chloro-5-cyano-4-(3-cyanophenyl)-2-cyclopropylaminopyrimidine (0.36 g, 65%) as a yellow solid. MS m/z calculated for (M+H)⁺ 296, found 296.

Step c: 5-Amino-4-(3-cyanophenyl)-2-cyclopropylamino-thieno[2,3-d]pyrimidine-6-carboxamide. 6-Chloro-5-cyano-4-(3-cyanophenyl)-2-cyclopropylaminopyrimidine (0.36 g, 1.22 mmol), 2-mercaptoacetamide (0.11 g, 1.22 mmol), and sodium carbonate (0.13 g, 1.22 mmol) were stirred in ethanol (10 mL) at 60° C. for 4 hours. The reaction mixture was partitioned between ethyl acetate and water. The organic layer was separated and concentrated to give a residue. Excess sodium ethoxide in ethanol was added and the mixture was heated at 75° C. for 2 hours. The reaction mixture was partitioned between ethyl acetate and water. The organic layer was separated and concentrated to give a residue which was purified using preparative HPLC to give the title compound 5-amino-4-(3-cyanophenyl)-2-cyclopropylamino-thieno[2,3-d]pyrimidine-6-carboxamide (0.18 g, 43%) as a yellow solid. NMR (DMSO-d₆): δ 7.73-8.01 (m, 5H), 7.03 (s, 2H), 6.05 (s, 2H), 2.80 (m, 1H), 0.71 (t, 2H), 0.51 (t, 2H). MS m/z calculated for (M+H)⁺ 351, found 351.

### 5.2.24 5-Amino-4-(3-pyridyl)-2-cyclopropylamino-thieno[2,3-d]pyrimidine-6-carboxamide

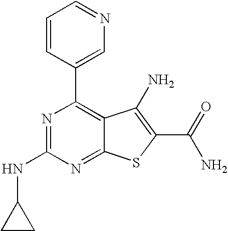

Step a: 5-Cyano-4-(3-pyridyl)-2-cyclopropylamino-6-oxopyrimidine. 3-Pyridinecarboxaldehyde (1.34 g, 12.6 mmol), ethylcyanoacetate (1.42 g, 12.6 mmol), N-cyclopropylguanidine.HCl (1.70 g, 12.6 mmol), and potassium carbonate (1.92 g, 13.9 mmol) were stirred in ethanol (50 mL) at 75° C. for 18 hours. The reaction mixture was partitioned between ethyl acetate and water. The organic layer was separated and concentrated to give a residue which was purified using flash chromatography (100% EtOAc) to give 5-cyano-4-(3-pyridyl)-2-cyclopropylamino-6-oxopyrimidine (1.12 g, 35%). MS m/z calculated for (M+H)⁺ 254, found 254.

Step b: 6-Chloro-5-cyano-4-(3-pyridyl)-2-cyclopropylaminopyrimidine. 5-Cyano-4-(3-pyridyl)-2-cyclopropylamino-6-oxopyrimidine (1.12 g, 4.43 mmol) was stirred in POCl₃ (10 mL). The mixture was heated at 90° C. for 2 hours. and then volatiles were concentrated off and the residue was partitioned between ethyl acetate and sat. NaHCO₃. The organic layer was separated and concentrated to give a residue which was recrystallized from ethanol to give 6-chloro-5-cyano-4-(3-pyridyl)-2-cyclopropylaminopyrimidine (0.73 g, 61%) as a yellow solid. MS m/z calculated for (M+H)⁺ 272, found 272.

Step c: 5-Amino-4-(3-pyridyl)-2-cyclopropylamino-thieno[2,3-d]pyrimidine-6-carboxamide. 6-Chloro-5-cyano-4-(3-pyridyl)-2-cyclopropylaminopyrimidine (0.73 g, 2.82 mmol), 2-mercaptoacetamide (0.26 g, 2.82 mmol), and sodium carbonate (0.30 g, 2.82 mmol) were stirred in ethanol (20 mL) at 60° C. for 4 hours. The reaction mixture was partitioned between ethyl acetate and water. The organic layer was separated and concentrated to give a residue. Excess sodium ethoxide in ethanol was added and the mixture was heated at 75° C. for 2 hours. The reaction mixture was partitioned between ethyl acetate and water. The organic layer was separated and concentrated to give a residue which was purified using preparative HPLC to give the title compound 5-amino-4-(3-pyridyl)-2-cyclopropylamino-thieno[2,3-d]pyrimidine-6-carboxamide (0.22 g, 24%) as a yellow solid. NMR (DMSO-d₆): δ 8.74 (m, 2H), 8.00 (m, 2H), 7.55 (m, 2H), 7.04 (s, 2H), 6.03 (s, 2H), 2.82 (m, 1H), 0.72 (t, 2H), 0.53 (t, 2H). MS m/z calculated for (M+H)⁺ 327, found 327.

### 5.2.25 5-Amino-4-(3-benzamide)-2-cyclopropylamino-thieno[2,3-d]pyrimidine-6-carboxamide

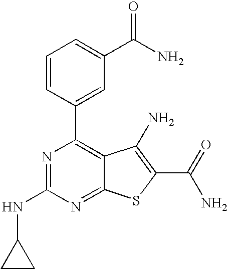

5-Amino-4-(3-cyanophenyl)-2-cyclopropylamino-thieno[2,3-d]pyrimidine-6-carboxamide (0.30 g, 0.86 mmol) was stirred in conc. sulfuric acid (1.2 mL) at 90° C. for 2 hours. The reaction was cooled, diluted with cold water, and partitioned between ethyl acetate and sat. NaHCO₃ to give a residue which was purified using preparative HPLC to give the title compound 5-Amino-4-(3-benzamide)-2-cyclopropylamino-thieno[2,3-d]pyrimidine-6-carboxamide (0.17 g, 54%) as a yellow solid. NMR (DMSO-d₆): δ 8.00-8.10 (m, 4H), 7.72 (d, 1H), 7.65 (t, 1H), 7.48 (s, 1H), 7.00 (s, 2H), 5.96 (s, 2H), 2.82 (m, 1H), 0.72 (m, 2H), 0.52 (t, 2H). MS m/z calculated for (M+H)⁺ 369, found 369.

### 5.2.26 5-Amino-4-(2-napthyl)-2-cyclopropylamino-thieno[2,3-d]pyrimidine-6-carboxamide

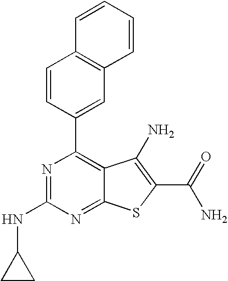

Step a: 5-Cyano-4-(2-napthyl)-2-cyclopropylamino-6-oxopyrimidine. 2-Napthaldehyde (2.03 g, 13.0 mmol), ethylcyanoacetate (1.47 g, 13.0 mmol), N-cyclopropylguanidine.HCl (1.76 g, 13.0 mmol), and potassium carbonate (1.98 g, 14.3 mmol) were stirred in ethanol (70 mL) at 75° C. for 18 hours. The reaction mixture was partitioned between ethyl acetate and water. The organic layer was separated and concentrated to give a residue which was triturated with ethyl acetate-hexanes to give 5-cyano-4-(2-napthyl)-2-cyclopropylamino-6-oxopyrimidine (2.00 g, 51%). MS m/z calculated for (M+H)⁺ 303, found 303.

Step b: 6-Chloro-5-cyano-4-(2-napthyl)-2-cyclopropylaminopyrimidine. 5-Cyano-4-(2-napthyl)-2-cyclopropylamino-6-oxopyrimidine (2.00 g, 6.62 mmol) was stirred in POCl₃ (20 mL). The mixture was heated at 90° C. for 2 hours. and then volatiles were concentrated off and the residue was partitioned between ethyl acetate and sat. NaHCO₃. The organic layer was separated and concentrated to give a residue which was recrystallized from ethanol to give 6-chloro-5-cyano-4-(2-napthyl)-2-cyclopropylaminopyrimidine (1.17 g, 55%) as a yellow solid. MS m/z calculated for (M+H)⁺ 321, found 321.

Step c: 5-Amino-4-(2-napthyl)-2-cyclopropylamino-thieno[2,3-d]pyrimidine-6-carboxamide. 6-Chloro-5-cyano-4-(2-napthyl)-2-cyclopropylaminopyrimidine (0.70 g, 2.19 mmol), 2-mercaptoacetamide (0.20 g, 2.19 mmol), and sodium carbonate (0.23 g, 2.19 mmol) were stirred in ethanol (25 mL) at 60° C. for 4 hours. The reaction mixture was partitioned between ethyl acetate and water. The organic layer was separated and concentrated to give a residue. Excess sodium ethoxide in ethanol was added and the mixture was heated at 75° C. for 2 hours. The reaction mixture was partitioned between ethyl acetate and water. The organic layer was separated and concentrated to give a residue which was purified using preparative HPLC to give the title compound 5-amino-4-(2-napthyl)-2-cyclopropylamino-thieno[2,3-d]pyrimidine-6-carboxamide (0.39 g, 48%) as a yellow solid. NMR (DMSO-d₆): δ 7.61-8.16 (m, 8H), 6.99 (s, 2H), 6.02 (s, 2H), 2.85 (m, 1H), 0.72 (d, 2H), 0.54 (t, 2H). MS m/z calculated for (M+H)⁺ 376, found 376.

### 5.2.27 5-Amino-4-(3,4-difluorophenyl)-2-cyclopropylamino-thieno[2,3-d]pyrimidine-6-carboxamide

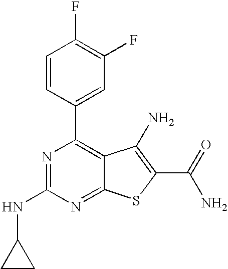

Step a: 5-Cyano-4-(3,4-difluorophenyl)-2-cyclopropylamino-6-oxopyrimidine. 3,4-Difluorobenzaldehyde (1.60 g, 11.3 mmol), ethylcyanoacetate (1.28 g, 11.3 mmol), N-cyclopropylguanidine.HCl (1.52 g, 11.3 mmol), and potassium carbonate (1.72 g, 12.4 mmol) were stirred in ethanol (75 mL) at 75° C. for 18 hours. The reaction mixture was partitioned between ethyl acetate and water. The organic layer was separated and concentrated to give a residue which was purified using flash chromatography (100% EtOAc) to give 5-cyano-4-(3,4-difluorophenyl)-2-cyclopropylamino-6-oxopyrimidine (1.13 g, 35%). MS m/z calculated for (M+H)⁺ 289, found 289.

Step b: 6-Chloro-5-cyano-4-(3,4-difluorophenyl)-2-cyclopropylaminopyrimidine. 5-Cyano-4-(3,4-difluorophenyl)-2-cyclopropylamino-6-oxopyrimidine (1.13 g, 3.92 mmol) was stirred in POCl₃ (15 mL). The mixture was heated at 90° C. for 2 hours. and then volatiles were concentrated off and the residue was partitioned between ethyl acetate and sat. NaHCO₃. The organic layer was separated and concentrated to give a residue which was purified using flash chromatography (25% EtOAc/hexanes) to give 6-chloro-5-cyano-4-(3,4-difluoro)-2-cyclopropylaminopyrimidine (1.03 g, 86%) as a yellow solid. MS m/z calculated for (M+H)⁺ 307, found 307.

Step c: 5-Amino-4-(3,4-difluorophenyl)-2-cyclopropylamino-thieno[2,3-d]pyrimidine-6-carboxamide. 6-Chloro-5-cyano-4-(3,4-difluorophenyl)-2-cyclopropylaminopyrimidine (1.03 g, 3.37 mmol), 2-mercaptoacetamide (0.31 g, 3.37 mmol), and sodium carbonate (0.36 g, 3.37 mmol) were stirred in ethanol (15 mL) at 60° C. for 4 hours. The reaction mixture was partitioned between ethyl acetate and water. The organic layer was separated and concentrated to give a residue. Excess sodium ethoxide in ethanol was added and the mixture was heated at 75° C. for 2 hours. The reaction mixture was partitioned between ethyl acetate and water. The organic layer was separated and concentrated to give a residue which was purified using preparative HPLC to give the title compound 5-amino-4-(3,4-difluorophenyl)-2-cyclopropylamino-thieno[2,3-d]pyrimidine-6-carboxamide (0.24 g, 20%) as a yellow solid. NMR (DMSO-d₆): δ 7.40-8.00 (m, 4H), 7.02 (s, 2H), 6.11 (s, 2H), 2.80 (m, 1H), 0.72 (t, 2H), 0.52 (t, 2H). MS m/z calculated for (M+H)⁺ 362, found 362.

### 5.2.28 5-Amino-4-(3-chloro-5-methoxyphenyl)-2-cyclopropylamino-thieno[2,3-d]pyrimidine-6-carboxamide

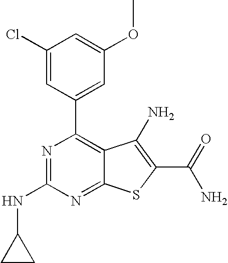

Step a: 5-Cyano-4-(3-chloro-5-methoxyphenyl)-2-cyclopropylamino-6-oxopyrimidine. 3-chloro-5-methoxybenzaldehyde (3.04 g, 17.9 mmol), ethylcyanoacetate (2.02 g, 17.9 mmol), N-cyclopropylguanidine.HCl (2.42 g, 17.9 mmol), and potassium carbonate (2.72 g, 19.7 mmol) were stirred in ethanol (100 mL) at 75° C. for 18 hours. The reaction mixture was partitioned between ethyl acetate and water. The organic layer was separated and concentrated to give a residue which was purified using flash chromatography (100% EtOAc) to give 5-cyano-4-(3-chloro-5-methoxyphenyl)-2-cyclopropylamino-6-oxopyrimidine (1.95 g, 35%). MS m/z calculated for (M+H)⁺ 317, found 317.

Step b: 6-Chloro-5-cyano-4-(3-chloro-5-methoxyphenyl)-2-cyclopropylaminopyrimidine. 5-Cyano-4-(3-chloro-5-methoxyphenyl)-2-cyclopropylamino-6-oxopyrimidine (1.30 g, 4.11 mmol) was stirred in POCl₃ (10 mL). The mixture was heated at 90° C. for 2 hours. and then volatiles were concentrated off and the residue was partitioned between ethyl acetate and sat. NaHCO₃. The organic layer was separated and concentrated to give a residue which was purified using flash chromatography (50% EtOAc/hexanes) to give 6-chloro-5-cyano-4-(3-chloro-5-methoxyphenyl)-2-cyclopropylaminopyrimidine (0.85 g, 62%). MS m/z calculated for (M+H)⁺ 335, found 335.

Step c: 5-Amino-4-(3-chloro-5-methoxyphenyl)-2-cyclopropylamino-thieno[2,3-d]pyrimidine-6-carboxamide. 6-Chloro-5-cyano-4-(3-chloro-5-methoxyphenyl)-2-cyclopropylaminopyrimidine (0.80 g, 2.40 mmol), 2-mercaptoacetamide (0.21 g, 2.40 mmol), and sodium carbonate (0.25 g, 2.40 mmol) were stirred in ethanol (20 mL) at 60° C. for 4 hours. The reaction mixture was partitioned between ethyl acetate and water. The organic layer was separated and concentrated to give a residue. Excess sodium ethoxide in ethanol was added and the mixture was heated at 75° C. for 2 hours. The reaction mixture was partitioned between ethyl acetate and water. The organic layer was separated and concentrated to give a residue which was triturated with ethyl acetate-hexanes to give the title compound 5-amino-4-(3-chloro-5-methoxyphenyl)-2-cyclopropylamino-thieno[2,3-d]pyrimidine-6-carboxamide (0.35 g, 36%) as a yellow solid. NMR (DMSO-d₆): δ 7.97 (s, 1H), 7.21 (m, 2H), 7.10 (s, 1H), 6.99 (s, 2H), 6.07 (s, 2H), 3.82 (s, 3H), 2.81 (m, 1H), 0.71 (d, 2H), 0.51 (t, 2H). MS m/z calculated for (M+H)⁺ 390, found 390.

### 5.2.29 5-Amino-4-(3-chloro-5-hydroxyphenyl)-2-cyclopropylamino-thieno[2,3-d]pyrimidine-6-carboxamide

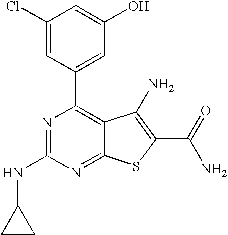

5-amino-4-(3-chloro-5-methoxyphenyl)-2-cyclopropylamino-thieno[2,3-d]pyrimidine-6-carboxamide (0.33 g, 0.85 mmol) was stirred in dichloromethane (15 mL) at 0° C. Boron tribromide (6 mL, 1M in dichloromethane) was slowly added and the reaction was warmed to room temperature over 3 hours. The mixture was quenched with ice and partitioned between ethyl acetate and sat. NaHCO₃. The organic layer was separated and concentrated to give a residue which was purified using preparative HPLC to give the title compound 5-Amino-4-(3-chloro-5-hydroxyphenyl)-2-cyclopropylamino-thieno[2,3-d]pyrimidine-6-carboxamide (0.16 g, 52%) as a yellow solid. NMR (DMSO-d₆): δ 10.3 (s, 1H), 7.95 (s, 1H), 6.97 (m, 4H), 6.87 (s, 1H), 6.08 (s, 2H), 2.85 (m, 1H), 0.71 (d, 2H), 0.51 (t, 2H). MS m/z calculated for (M+H)⁺ 376, found 376.

### 5.2.30 5-Amino-4-(3-chlorophenyl)-2-cyclopropylamino-thieno[2,3-d]pyrimidine-6-carboxamide

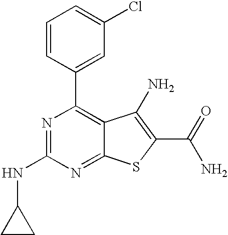

Step a: 5-Cyano-4-(3-chlorophenyl)-2-cyclopropylamino-6-oxopyrimidine. 3-Chlorobenzaldehyde (2.60 g, 18.5 mmol), ethylcyanoacetate (2.09 g, 18.5 mmol), N-cyclopropylguanidine.HCl (2.50 g, 18.5 mmol), and potassium carbonate (2.81 g, 20.3 mmol) were stirred in ethanol (80 mL) at 75° C. for 18 hours. The reaction mixture was partitioned between ethyl acetate and water. The organic layer was separated and concentrated to give a residue which was purified using flash chromatography (100% EtOAc) to give 5-cyano-4-(3-chlorophenyl)-2-cyclopropylamino-6-oxopyrimidine (2.91 g, 55%). MS m/z calculated for (M+H)⁺ 287, found 287.

Step b: 6-Chloro-5-cyano-4-(3-chlorophenyl)-2-cyclopropylaminopyrimidine. 5-Cyano-4-(3-chlorophenyl)-2-cyclopropylamino-6-oxopyrimidine (2.91 g, 10.1 mmol) was stirred in POCl₃ (7 mL). The mixture was heated at 90° C. for 2 hours. and then volatiles were concentrated off and the residue was partitioned between ethyl acetate and sat. NaHCO₃. The organic layer was separated and concentrated to give a residue which was recrystallized from ethanol to give 6-chloro-5-cyano-4-(3-chlorophenyl)-2-cyclopropylaminopyrimidine (2.16 g, 70%) as a yellow solid. MS m/z calculated for (M+H)⁺ 305, found 305.

Step c: 5-Amino-4-(3-chlorophenyl)-2-cyclopropylamino-thieno[2,3-d]pyrimidine-6-carboxamide. 6-Chloro-5-cyano-4-(3-chlorophenyl)-2-cyclopropylaminopyrimidine (2.16 g, 7.10 mmol), 2-mercaptoacetamide (0.64 g, 7.10 mmol), and sodium carbonate (0.75 g, 7.10 mmol) were stirred in ethanol (30 mL) at 60° C. for 4 hours. The reaction mixture was partitioned between ethyl acetate and water. The organic layer was separated and concentrated to give a residue. Excess sodium ethoxide in ethanol was added and the mixture was heated at 75° C. for 2 hours. The reaction mixture was partitioned between ethyl acetate and water. The organic layer was separated and concentrated to give a residue which was purified using preparative HPLC to give the title compound 5-amino-4-(3-chlorophenyl)-2-cyclopropylamino-thieno[2,3-d]pyrimidine-6-carboxamide (0.45 g, 18%) as a yellow solid. NMR (DMSO-d₆): δ 7.95 (s, 1H), 7.54-7.65 (m, 4H), 7.01 (s, 2H), 6.02 (s, 2H), 2.83 (m, 1H), 0.71 (t, 2H), 0.51 (t, 2H). MS m/z calculated for (M+H)⁺ 360, found 360.

### 5.2.31 5-Amino-4-(3-pyridyl-4-methoxy)-2-cyclopropylamino-thieno[2,3-d]pyrimidine-6-carboxamide

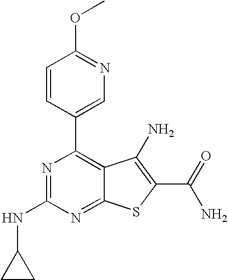

Step a: 5-Cyano-4-(3-pyridyl-4-methoxy)-2-cyclopropylamino-6-oxopyrimidine. 6-Methoxy-3-pyridinecarboxaldehyde (2.53 g, 18.5 mmol), ethylcyanoacetate (2.09 g, 18.5 mmol), N-cyclopropylguanidine.HCl (2.50 g, 18.5 mmol), and potassium carbonate (2.81 g, 20.4 mmol) were stirred in ethanol (85 mL) at 75° C. for 18 hours. The reaction mixture was partitioned between ethyl acetate and water. The organic layer was separated and concentrated to give a residue which was triturated with ethyl acetate-hexanes to give 5-cyano-4-(3-pyridyl-4-methoxy)-2-cyclopropylamino-6-oxopyrimidine (1.71 g, 33%). MS m/z calculated for (M+H)⁺ 284, found 284.

Step b: 6-Chloro-5-cyano-4-(3-pyridyl-4-methoxy)-2-cyclopropylaminopyrimidine. 5-Cyano-4-(3-pyridyl-4-methoxy)-2-cyclopropylamino-6-oxopyrimidine (1.70 g, 6.00 mmol) was stirred in POCl₃ (10 mL). The mixture was heated at 90° C. for 2 hours and then volatiles were concentrated off and the residue was partitioned between ethyl acetate and sat. NaHCO₃. The organic layer was separated and concentrated to give 6-chloro-5-cyano-4-(3-pyridyl-4-methoxy)-2-cyclopropylaminopyrimidine (1.72 g, 95%) as a yellow solid. MS m/z calculated for (M+H)⁺ 302, found 302.

Step c: 5-Amino-4-(3-pyridyl-4-methoxy)-2-cyclopropylamino-thieno[2,3-d]pyrimidine-6-carboxamide. 6-Chloro-5-cyano-4-(3-pyridyl-4-methoxy)-2-cyclopropylaminopyrimidine (1.72 g, 5.71 mmol), 2-mercaptoacetamide (0.52 g, 5.71 mmol), and sodium carbonate (0.60 g, 5.71 mmol) were stirred in ethanol (15 mL) at 60° C. for 4 hours. The reaction mixture was partitioned between ethyl acetate and water. The organic layer was separated and concentrated to give a residue. Excess sodium ethoxide in ethanol was added and the mixture was heated at 75° C. for 2 hours. The reaction mixture was partitioned between ethyl acetate and water. The organic layer was separated and concentrated to give a residue which was purified using preparative HPLC to give the title compound 5-amino-4-(3-pyridyl-4-methoxy)-2-cyclopropylamino-thieno[2,3-d]pyrimidine-6-carboxamide (0.61 g, 30%) as a yellow solid. NMR (DMSO-d₆): δ 8.50 (s, 1H), 8.00 (m, 2H), 7.00-7.09 (m, 3H), 6.30 (s, 2H), 4.02 (s, 3H), 2.90 (m, 1H), 0.72 (t, 2H), 0.53 (t, 2H). MS m/z calculated for (M+H)⁺ 357, found 357.

### 5.2.32 5-Amino-4-(3-methoxy-4-methylphenyl)-2-cyclopropylamino-thieno[2,3-d]pyrimidine-6-carboxamide

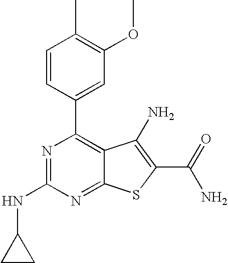

Step a: 5-Cyano-4-(3-methoxy-4-methylphenyl)-2-cyclopropylamino-6-oxopyrimidine. 3-Methoxy-4-methylbenzaldehyde (3.20 g, 21.3 mmol), ethylcyanoacetate (2.41 g, 21.3 mmol), N-cyclopropylguanidine.HCl (2.87 g, 21.3 mmol), and potassium carbonate (3.24 g, 23.4 mmol) were stirred in ethanol (100 mL) at 75° C. for 18 hours. The reaction mixture was partitioned between ethyl acetate and water. The organic layer was separated and concentrated to give a residue which was purified using flash chromatography (100% EtOAc) to give 5-cyano-4-(3-methoxy-4-methylphenyl)-2-cyclopropylamino-6-oxopyrimidine (1.82 g, 29%). MS m/z calculated for (M+H)⁺ 297, found 297.

Step b: 6-Chloro-5-cyano-4-(3-methoxy-4-methylphenyl)-2-cyclopropylaminopyrimidine. 5-Cyano-4-(3-methoxy-4-methylphenyl)-2-cyclopropylamino-6-oxopyrimidine (1.60 g, 5.40 mmol) was stirred in POCl₃ (5 mL). The mixture was heated at 90° C. for 2 hours. and then volatiles were concentrated off and the residue was partitioned between ethyl acetate and sat. NaHCO₃. The organic layer was separated and concentrated to give quantitative 6-chloro-5-cyano-4-(3-methoxy-4-methylphenyl)-2-cyclopropylaminopyrimidine (1.70 g) as a yellow solid. MS m/z calculated for (M+H)⁺ 315, found 315.

Step c: 5-Amino-4-(3-methoxy-4-methylphenyl)-2-cyclopropylamino-thieno[2,3-d]pyrimidine-6-carboxamide. 6-Chloro-5-cyano-4-(3-methoxy-4-methylphenyl)-2-cyclopropylaminopyrimidine (1.70 g, 5.40 mmol), 2-mercaptoacetamide (0.49 g, 5.40 mmol), and sodium carbonate (0.57 g, 5.40 mmol) were stirred in ethanol (20 mL) at 60° C. for 4 hours. The reaction mixture was partitioned between ethyl acetate and water. The organic layer was separated and concentrated to give a residue. Excess sodium ethoxide in ethanol was added and the mixture was heated at 75° C. for 2 hours. The reaction mixture was partitioned between ethyl acetate and water. The organic layer was separated and concentrated to give a residue which was purified using preparative HPLC to give the title compound 5-amino-4-(3-methoxy-4-methylphenyl)-2-cyclopropylamino-thieno[2,3-d]pyrimidine-6-carboxamide (1.10 g, 55%) as a yellow solid. NMR (DMSO-d₆): δ 7.90 (s, 1H), 7.35 (d, 1H), 6.95-7.20 (m, 4H), 6.05 (s, 2H), 3.82 (s, 3H), 2.80 (m, 1H), 2.23 (s, 3H), 0.70 (t, 2H), 0.50 (t, 2H). MS m/z calculated for (M+H)⁺ 370, found 370.

### 5.2.33 5-Amino-4-(3-hydroxy-4-methylphenyl)-2-cyclopropylamino-thieno[2,3-d]pyrimidine-6-carboxamide

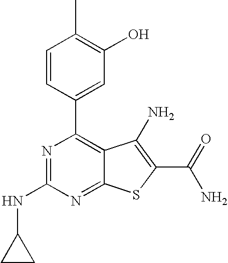

5-amino-4-(3-methoxy-4-methylphenyl)-2-cyclopropylamino-thieno[2,3-d]pyrimidine-6-carboxamide (0.70 g, 1.90 mmol) was stirred in dichloromethane (40 mL) at 0° C. Boron tribromide (14 mL, 1M in dichloromethane) was slowly added and the reaction was warmed to room temperature over 3 hours. The mixture was quenched with ice and partitioned between ethyl acetate and sat. NaHCO₃. The organic layer was separated and concentrated to give a residue which was purified using preparative HPLC to give the title compound 5-Amino-4-(3-hydroxy-4-methylphenyl)-2-cyclopropylamino-thieno[2,3-d]pyrimidine-6-carboxamide (0.17 g, 25%) as a yellow solid. NMR (DMSO-d₆): δ 9.73 (s, 1H), 7.90 (s, 1H), 7.25 (d, 1H), 6.80-7.00 (m, 4H), 6.15 (s, 2H), 2.80 (m, 1H), 2.20 (s, 3H), 0.70 (t, 2H), 0.50 (t, 2H). MS m/z calculated for (M+H)⁺ 356, found 356.

### 5.2.34 5-Amino-4-(methyl-4-benzoate)-2-cyclopropylamino-thieno[2,3-d]pyrimidine-6-carboxamide

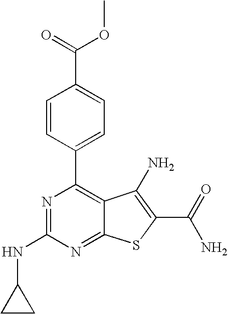

Step a: 5-Cyano-4-(methyl-4-benzoate)-2-cyclopropylamino-6-oxopyrimidine. Methyl-4-formylbenzoate (2.43 g, 14.8 mmol), ethylcyanoacetate (1.67 g, 14.8 mmol), N-cyclopropylguanidine.HCl (2.00 g, 14.8 mmol), and potassium carbonate (2.25 g, 16.3 mmol) were stirred in ethanol (70 mL) at 75° C. for 18 hours. The reaction mixture was partitioned between ethyl acetate and water. The organic layer was separated and concentrated to give a residue which was purified using flash chromatography (100% EtOAc) to give 5-cyano-4-(ethyl-4-benzoate)-2-cyclopropylamino-6-oxopyrimidine (0.61 g, 13%). MS m/z calculated for (M+H)⁺ 312, found 312.

Step b: 6-Chloro-5-cyano-4-(2-methoxyphenyl)-2-cyclopropylaminopyrimidine. 5-Cyano-4-(ethyl-4-benzoate)-2-cyclopropylamino-6-oxopyrimidine (0.61 g, 1.96 mmol) was stirred in POCl₃ (7 mL). The mixture was heated at 90° C. for 2 hours and then volatiles were concentrated off and the residue was partitioned between ethyl acetate and sat. NaHCO₃. The organic layer was separated and concentrated to give quantitative 6-chloro-5-cyano-4-(ethyl-4-benzoate)-2-cyclopropylaminopyrimidine (0.65 g) as a yellow solid. MS m/z calculated for (M+H)⁺ 330, found 330.

Step c: 5-Amino-4-(methyl-4-benzoate)-2-cyclopropylamino-thieno[2,3-d]pyrimidine-6-carboxamide. 6-Chloro-5-cyano-4-(ethyl-4-benzoate)-2-cyclopropylaminopyrimidine (0.65 g, 1.97 mmol), 2-mercaptoacetamide (0.18 g, 1.97 mmol), and sodium carbonate (0.21 g, 1.97 mmol) were stirred in ethanol (15 mL) at 60° C. for 4 hours. The reaction mixture was partitioned between ethyl acetate and water. The organic layer was separated and concentrated to give a residue. Excess sodium ethoxide in ethanol was added and the mixture was heated at 75° C. for 2 hours. The reaction mixture was diluted with water and acidified to pH 3 with 2 N HCl. The resulting crude solid was filtered, dried, and then taken up in methanol (20 mL) with a few drops of conc. sulfuric acid. The mixture was refluxed overnight, cooled, and partitioned between ethyl acetate and sat. NaHCO₃. The organic layer was separated and concentrated to give a residue which was purified using preparative HPLC to give the title compound 5-amino-4-(methyl-4-benzoate)-2-cyclopropylamino-thieno[2,3-d]pyrimidine-6-carboxamide (0.35 g, 48%) as a yellow solid. NMR (DMSO-d₆): δ 7.71-8.12 (m, 5H), 7.01 (s, 2H), 5.97 (s, 2H), 3.91 (s, 3H), 2.80 (m, 1H), 0.71 (t, 2H), 0.52 (t, 2H). MS m/z calculated for (M+H)⁺ 384, found 384.

### 5.2.35 5-Amino-4-(4-hydroxymethylphenyl)-2-cyclopropylamino-thieno[2,3-d]pyrimidine-6-carboxamide

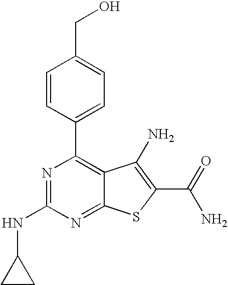

5-amino-4-(methyl-4-benzoate)-2-cyclopropylamino-thieno[2,3-d]pyrimidine-6-carboxamide (0.20 g, 0.54 mmol) was stirred in DME (4 mL) at 50° C. Sodium borohydride (0.11 g, 2.91 mmol) was added followed by the addition of methanol (2 mL). The reaction mixture was heated at 60° C. for 4 hours, then cooled and partitioned between ethyl acetate and water. The organic layer was separated and concentrated to give a residue which was purified using preparative HPLC to give the title compound 5-amino-4-(4-hydroxymethylphenyl)-2-cyclopropylamino-thieno[2,3-d]pyrimidine-6-carboxamide (0.04 g, 21%) as a yellow solid. NMR (DMSO-d₆): δ 7.95 (s, 1H), 7.47-7.52 (m, 4H), 6.97 (s, 2H), 5.98 (s, 2H), 5.33 (t, 1H), 4.61 (d, 2H), 2.80 (m, 1H), 0.71 (t, 2H), 0.52 (t, 2H). MS m/z calculated for (M+H)⁺ 356, found 356.

### 5.2.36 5-Amino-4-(3,4-dichloro-phenyl)-2-(2-hydroxy-1-methyl-ethylamino)-thieno[2,3-d]pyrimidine-6-carboxylic acid amide

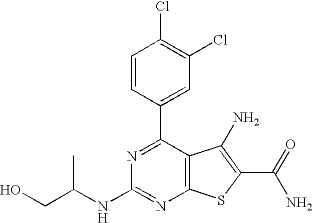

5-amino-4-(3,4-dichlorophenyl)-2-(methylsulfinyl)thiopheno[2,3-d]pyrimidine-6-carboxamide (125 mg, 0.31 mmol) and 2-amino-1-propanol (68.3 mg, 0.93 mmol) were placed in a 4 mL glass vial and heated at 90° C. overnight. The residue was suspended in DMSO and purified using preparative HPLC (20-100 MeCN, water) without buffer. The product was dried under vacuum to give 23 mg (18%) of the desired product as a yellow solid. ¹H NMR (400 MHz, D6-DMSO) δ ppm 7.85 (d, 1H, J=2), 7.79 (d, 1H, J=8), 7.58 (d, 1H, J=8), 7.00 (s, 2H), 6.14 (bs, 2H), 4.69 (m, 1H), 4.08 (m, 1H), 3.44 (m, 1H), 3.32 (m, 1H), 1.15 (d, 3H, J=7). MS m/z calculated for (M+H)⁺ 412, found 412.

### 5.2.37 5-Amino-2-cyclohexylamino-4-(3,4-dichloro-phenyl)-thieno[2,3-d]pyrimidine-6-carboxylic acid amide

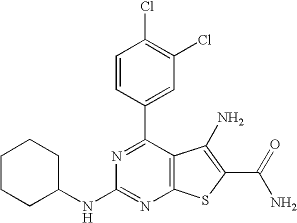

5-amino-4-(3,4-dichlorophenyl)-2-(methylsulfinyl)thiopheno[2,3-d]pyrimidine-6-carboxamide (125 mg, 0.31 mmol) and cyclohexyl amine (92 mg, 0.93 mmol) were placed in a 4 mL glass vial and heated at 90° C. overnight. The residue was suspended in Ethanol, and the precipitate was collected via vacuum filtration to give 20 mg (15%) of as a yellow solid. ¹H NMR (400 MHz, D6-DMSO) δ ppm 7.85 (d, 1H, J=2), 7.78 (m, 1H), 7.57 (d, 1H, J=8), 6.99 (s, 2H), 6.12 (bs, 2H), 3.78 (m, 1H), 1.89 (m, 2H), 1.71 (m, 2H), 1.59 (m, 1H), 1.28 (m, 4H), 1.11 (m, 1H). MS m/z calculated for (M+H)⁺ 436, found 436.

### 5.2.38 5-amino-2-(cyclopropylamino)-4-(2,3-dihydrobenzofuran-6-yl)thieno[2,3-d]pyrimidine-6-carboxamide

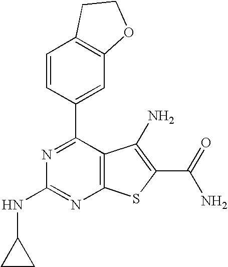

Step a: 2-(2,5-dibromophenoxy)ethanol. 1,4-dibromo-2-fluorobenzene (5.00 g, 19.7 mmol) and ethylene glycol (25 mL, 453 mmol) were dissolved in NMP (2.6 mL) with stirring at room temperature under N₂. KOtBu (7.73 g, 68.9 mmol) was added slowly. The resulting mixture was heated at 100° C. under a reflux condenser under N₂ for 6 hours and then cooled to room temperature overnight. H₂O (10 mL) was added slowly with vigorous stirring. The resulting mixture was filtered and the solids washed once with ethylene glycol (4 mL). H₂O (87 mL) was added to the filtrate and the resulting mixture cooled to 5° C. for 15 minutes. The solids were collected by vacuum filtration and washed 4 times with H₂O. The solids were dried on the filter with suction under N₂ for 20 hours and then dried further under high vacuum to yield 4.65 g (80%) of 2-(2,5-dibromophenoxy)ethanol as a light orange solid. ¹H NMR (300 MHz, DMSO-D6) δ ppm 3.73 (q, J=4.94 Hz, 2H) 4.07-4.14 (m, 2H) 4.90 (t, J=5.22 Hz, 1H) 7.08 (dd, J=8.24, 2.20 Hz, 1H) 7.33 (d, J=2.20 Hz, 1H) 7.52 (d, J=8.52 Hz, 1H). MS m/z calculated for (M+H)⁺ 296.89, found 297.

Step b: 6-bromo-2,3-dihydrobenzofuran. 2-(2,5-dibromophenoxy)ethanol (4.65 g, 15.7 mmol) was dissolved in toluene (33 mL) with stirring under N₂. PBr₃ (0.67 mL, 7.07 mmol) was added and the resulting mixture heated at 90° C. under reflux condenser under N₂ for 2.5 hours. The mixture was cooled to room temperature with a H₂O bath and 1N NaOH (28 mL) was added. The mixture was diluted with Et₂O, shaken, and the layers separated. The organics were washed with H₂O, dried over MgSO₄, filtered, and concentrated on a rotary evaporator. The resulting oil was dissolved in THF (15 mL) with stirring under N₂. The solution was cooled to −75° C. and n-BuLi (9.82 mL of a 1.6 M solution in hexane, 15.7 mmol) was added dropwise. The resulting clear, colorless solution was stirred 30 minutes at −75° C., and then HOAc (1 mL) was added. The resulting mixture was warmed to room temperature and partitioned between H₂O and Et₂O. The organics were washed once with 0.5 M NaOH, once with H₂O, and concentrated on a rotary evaporator. Flash chromatography (5-10% EtOAc/hexane) gave 1.38 g (90% purity) (40% yield) of 6-bromo-2,3-dihydrobenzofuran as a white solid. R_{f}=0.56 (30% EtOAc/hexane). ¹H NMR (300 MHz, DMSO-D6) δ ppm 3.13 (t, J=8.52 Hz, 2H) 4.56 (t, J=8.79 Hz, 2H) 6.95-7.01 (m, 2H) 7.14-7.20 (m, 1H).

Step c: 2,3-dihydrobenzofuran-6-carbaldehyde. 6-bromo-2,3-dihydrobenzofuran (1.38 g, 6.93 mmol) was dissolved in THF (9 mL) with stirring under N₂. The resulting solution was cooled to −75° C. and n-BuLi (4.8 mL of a 1.6 M solution in hexane, 7.63 mmol) was added dropwise. The resulting mixture was stirred 30 minutes at −75° C. (white solids form). DMF (1.1 mL, 13.9 mmol) was added dropwise (solids dissolve). The cold bath was removed and the reaction mixture allowed to warm to room temperature over 45 minutes. Saturated, aqueous NH₄Cl was added and the mixture extracted with EtOAc. The organics were washed once with brine, dried over MgSO₄, filtered, and concentrated on a rotary evaporator. Flash chromatography (5-10-20% EtOAc/hexane) gave 806 mg (79%) of 2,3-dihydrobenzofuran-6-carbaldehyde as a white solid. R_{f}=0.35 (30% EtOAc/hexane). ¹H NMR (300 MHz, DMSO-D6) δ ppm 3.27 (t, J=8.79 Hz, 2H) 4.60 (t, J=8.79 Hz, 2H) 7.19 (s, 1H) 7.40-7.50 (m, 2H) 9.90 (s, 1H). MS m/z calculated for (M+H)⁺ 149, found 149.

Step d: 2-(cyclopropylamino)-4-(2,3-dihydrobenzofuran-6-yl)-6-hydroxypyrimidine-5-carbonitrile. 2,3-dihydrobenzofuran-6-carbaldehyde (802 mg, 5.41 mmol), ethyl cyanoacetate (612 mg, 5.41 mmol), and piperidine (1 drop) were refluxed in toluene (7 mL) with azeotropic removal of H₂O under N₂. After 1.5 hours, ethyl cyanoacetate (2 drops) and piperidine (1 drop) were added and refluxing continued for another 30 minutes. The stirbar was removed and the volatiles removed on a rotary evaporator. To the resulting material was added sequentially: a stirbar, 1-cyclopropylguanidine hydrochloride (807 mg, 5.95 mmol), K₂CO₃ (1.65 g, 11.9 mmol), and ethanol (8 mL). The resulting mixture was stirred vigorously and heated at 75° C. under a reflux condenser under N₂ for 1.5 hours. Methanol and silica gel were added and the volatiles removed on a rotary evaporator. Flash chromatography (50-90% EtOAc/hexane-2-5-10% MeOH/CH₂Cl₂) gave a solid which was triturated with Et₂O and dried under high vacuum to give 605 mg (38%) of 2-(cyclopropylamino)-4-(2,3-dihydrobenzofuran-6-yl)-6-hydroxypyrimidine-5-carbonitrile as a light yellow solid. ¹H NMR (300 MHz, DMSO-D6) δ ppm 0.56-0.65 (m, 2H) 0.72-0.84 (m, 2H) 2.80-3.00 (m, 1H) 3.24 (t, J=8.79 Hz, 2H) 4.58 (t, J=8.79 Hz, 2H) 7.09-7.20 (m, 1H) 7.29-7.38 (m, 2H) 8.92 (s, 1H). MS m/z calculated for (M+H)⁺ 295, found 295.

Step e: 4-chloro-2-(cyclopropylamino)-6-(2,3-dihydrobenzofuran-6-yl)pyrimidine-5-carbonitrile. To 2-(cyclopropylamino)-4-(2,3-dihydrobenzofuran-6-yl)-6-hydroxypyrimidine-5-carbonitrile (602 mg, 2.05 mmol) in dioxane (10 mL) was added POCl₃ (5 mL). The resulting reaction mixture was heated at 90° C. under a reflux condenser under N₂ for 40 minutes and then cooled to room temperature. The volatiles were removed on a rotary evaporator. The resulting solids were triturated with dilute aqueous NaHCO₃ and collected by vacuum filtration. The solids were washed with H₂O and then 20% Et₂O in hexane and then dried under high vacuum to give 598 mg (93%) of 4-chloro-2-(cyclopropylamino)-6-(2,3-dihydrobenzofuran-6-yl)pyrimidine-5-carbonitrile as a yellow solid. ¹H NMR (300 MHz, DMSO-D6) δ ppm 0.53-0.63 (m, 2H) 0.70-0.81 (m, 2H) 2.80-3.00 (m, 1H) 3.21-3.33 (m, 2H) 4.55-4.66 (m, 2H) 7.26-7.35 (m, 1H) 7.36-7.48 (m, 2H) 8.94 (dd, J=15.52, 3.98 Hz, 1H). MS m/z calculated for (M+H)⁺ 313, found 313.

Step f: 2-(5-cyano-2-(cyclopropylamino)-6-(2,3-dihydrobenzofuran-6-yl)pyrimidin-4-ylthio)acetamide. 4-chloro-2-(cyclopropylamino)-6-(2,3-dihydrobenzofuran-6-yl)pyrimidine-5-carbonitrile (594 mg, 1.90 mmol), 2-mercaptoacetamide (182 mg, 1.99 mmol), K₂CO₃ (276 mg, 1.99 mmol), and ethanol (12 mL) were combined in a flask, stirred vigorously, and heated at 50° C. under a reflux condenser under N₂ for 16 hours. The reaction mixture was cooled to room temperature and 40 mL of ice water was added. The resulting solids were collected by vacuum filtration, washed once with H₂O, twice with 30% Et₂O/hexane, and dried under high vacuum at 50° C. to give 608 mg (100%) of 2-(5-cyano-2-(cyclopropylamino)-6-(2,3-dihydrobenzofuran-6-yl)pyrimidin-4-ylthio)acetamide as a light yellow solid. ¹H NMR (300 MHz, DMSO-D6) δ ppm 0.50-0.62 (m, 2H) 0.69-0.82 (m, 2H) 2.86-3.01 (m, 1H) 3.19-3.30 (m, 2H) 3.98 (s, 2H) 4.54-4.65 (m, 2H) 7.13 (s, 1H) 7.20 (s, 1H) 7.23-7.30 (m, 1H) 7.34-7.42 (m, 1H) 7.57 (s, 1H) 8.55 (d, J=4.12 Hz, 1H). MS m/z/z calculated for (M+H)⁺ 368, found 368.

Step g: 5-amino-2-(cyclopropylamino)-4-(2,3-dihydrobenzofuran-6-yl)thieno[2,3-d]pyrimidine-6-carboxamide. A stirred suspension of 2-(5-cyano-2-(cyclopropylamino)-6-(2,3-dihydrobenzofuran-6-yl)pyrimidin-4-ylthio)acetamide (605 mg, 1.65 mmol) in ethanol (6 mL) was cooled to 0° C. under N₂. Freshly prepared NaOEt (8.27 mmol) in ethanol (3 mL) was added and the resulting mixture stirred 2 hours while being slowly warmed to room temperature. The mixture was then heated at 50° C. for 18.5 hours. HCl (8.27 mmol) in 20 mL of ice water was added with vigorous stirring. The resulting solids were collected by vacuum filtration, washed once with H₂O, once with 20% Et₂O/hexane, and then dried under high vacuum. The resulting solids were triturated with boiling ethanol, collected by vacuum filtration, washed with ethanol, then Et₂O, and dried under high vacuum to give 417 mg (69%) of 5-amino-2-(cyclopropylamino)-4-(2,3-dihydrobenzofuran-6-yl)thieno[2,3-d]pyrimidine-6-carboxamide as a yellow solid. ¹H NMR (400 MHz, DMSO-D6) δ ppm 0.47-0.55 (m, 2H) 0.65-0.75 (m, 2H) 2.75-2.85 (m, 1H) 3.27 (t, J=8.79 Hz, 2H) 4.61 (t, J=8.79 Hz, 2H) 6.05 (s, 2H) 6.87-7.03 (m, 4H) 7.38 (d, J=7.42 Hz, 1H) 7.88 (s, 1H). MS m/z calculated for (M+H)⁺ 368, found 368.

### 5.2.39 (S)-5-amino-4-(3,4-dichlorophenyl)-2-(3-hydroxy-2-methylpropyl)thieno[2,3-d]pyrimidine-6-carboxamide

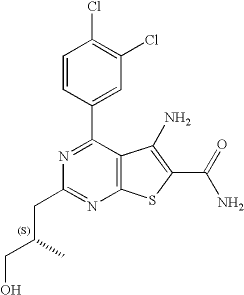

Step a: (R)-methyl3-(benzyloxy)-2-methylpropanoate. A stirred solution of (R)-methyl 3-hydroxy-2-methylpropanoate (9.57 g, 81.0 mmol) and benzyl 2,2,2-trichloroacetimidate (22.50 g, 89.1 mmol) in CH₂Cl₂ (10 mL) was cooled to 0° C. under N₂. Triflic acid (0.1 mL) in CH₂Cl₂ (1 mL) was added dropwise. After 10 minutes the reaction became a solid mass. After 1 hour, saturated aqueous NaHCO₃ was added and the mixture extracted with Et₂O. The organics were dried over MgSO₄, filtered and concentrated on a rotary evaporator. Hexane was added and the resulting solids collected by vacuum filtration. The filtrate was concentrated on a rotary evaporator and purified using flash chromatography (5-10% EtOAc/hexane) to give 10.39 g (62%) of (R)-methyl3-(benzyloxy)-2-methylpropanoate as a colorless oil. R_{f}=0.20 (10% EtOAc/hexane). ¹H NMR (300 MHz, DMSO-D6) δ ppm 1.08 (d, J=7.14 Hz, 3H) 2.75 (ddd, J=14.01, 12.64, 7.14 Hz, 1H) 3.44-3.59 (m, 2H) 3.60 (s, 3H) 4.46 (s, 2H) 7.23-7.40 (m, 5H).

Step b: (S)-3-(benzyloxy)-2-methylpropan-1-ol. A vigorously stirred suspension of LiAlH₄ (3.59 g, 94.5 mmol) in THF (30 mL) was cooled to −75° C. under N₂. (R)-methyl 3-(benzyloxy)-2-methylpropanoate (9.37 g, (45.0 mmol) in THF (10 mL) was added dropwise via pipette. The resulting reaction mixture was stirred 30 minutes at −75° C. Saturated aqueous Na₂SO₄ was added dropwise via addition funnel until all LiAlH₄ was quenched (salts turn white). The resulting mixture was filtered and the filter cake washed twice with Et₂O. The filtrate was concentrated on a rotary evaporator and purified using flash chromatography (10-30% EtOAc/hexane) to give 6.49 g (80%) of (S)-3-(benzyloxy)-2-methylpropan-1-ol as a colorless oil. R_{f}=0.23 (30% EtOAc/hexane). ¹H NMR (300 MHz, DMSO-D6) δ ppm 0.86 (d, J=6.87 Hz, 3H) 1.74-1.88 (m, 1H) 3.20-3.45 (m, 4H) 4.38-4.47 (m, 3H) 7.22-7.40 (m, 5H). MS m/z calculated for (M+Na)⁺ 203, found 203.

Step c: (S)-4-(benzyloxy)-3-methylbutanenitrile. (S)-3-(benzyloxy)-2-methylpropan-1-ol (1.25 g, 6.94 mmol) and triethylamine (1.93 mL, 13.9 mmol) were dissolved in CH₂Cl₂ (10 mL) with stirring under N₂ and cooled to 0° C. Methanesulfonyl chloride (0.59 mL, 7.63 mmol) in CH₂Cl₂ (2 mL) was added dropwise. The resulting mixture (cloudy light yellow) was stirred 40 minutes at 0° C. and then all volatiles were removed on a rotary evaporator. DMSO (10 mL), NaCN (1.02 g, 20.8 mmol), and dimethylaminopyridine (0.01 g, 0.07 mmol) were added and the resulting mixture stirred vigorously and heated at 50° C. for 24 hours. H₂O was added and the mixture extracted with Et₂O. The organics were washed twice with H₂O, once with brine, and concentrated on a rotary evaporator. Flash chromatography (10-20% EtOAc/hexane) gave 993 mg (76%) of (S)-4-(benzyloxy)-3-methylbutanenitrile as a colorless oil. R_{f}=0.39 (30% EtOAc/hexane). ¹H NMR (300 MHz, DMSO-D6) δ ppm 0.98 (d, J=6.59 Hz, 3H) 2.01-2.19 (m, 1H) 2.41-2.64 (m, 2H) 3.23-3.41 (m, 2H) 4.48 (s, 2H) 7.20-7.44 (m, 5H). MS m/z calculated for (M+H)⁺ 190, found 190.

Step d: (S)-4-(benzyloxy)-3-methylbutanimidamide. (S)-4-(benzyloxy)-3-methylbutanenitrile (0.988 g, 5.22 mmol) was dissolved in ethanol (10 mL) with stirring and cooled to 0° C. HCl gas was bubbled in for 30 minutes and then the flask was capped and stirred at 0° C. for 4 hours. All volatiles were removed on a rotary evaporator for 30 minutes. Meanwhile NH₃ gas was bubbled into ethanol (19 mL) at 0° C. for 20 minutes. The resulting NH₃/ethanol solution was poured onto the above-prepared crude imidate at 0° C. The resulting mixture was stirred vigorously while being slowly warmed to room temperature for 16 hours. All volatiles were removed on a rotary evaporator. Toluene (10 mL) was added and then removed on a rotary evaporator at 65° C. for 30 minutes. This material was used in the next step without purification. MS m/z calculated for (M+H)⁺ 207, found 207.

Step e: (S)-2-(3-(benzyloxy)-2-methylpropyl)-4-(3,4-dichlorophenyl)-6-hydroxypyrimidine-5-carbonitrile. 3,4-dichlorobenzaldehyde (914 mg, 5.22 mmol), ethyl cyanoacetate (591 mg, 5.22 mmol), and piperidine (1 drop) were refluxed in toluene (9 mL) with azeotropic removal of H₂O under N₂ for 30 minutes. The stirbar was removed and the volatiles removed on a rotary evaporator. To the resulting material was added sequentially: a stirbar, (S)-4-(benzyloxy)-3-methylbutanimidamide (5.22 mmol crude), K₂CO₃ (1.44 g, 10.4 mmol), and ethanol (19 mL). The resulting mixture was stirred vigorously and heated at 75° C. under a reflux condenser under N₂ for 4.5 hours. Heating was discontinued and the resulting mixture partitioned between ice water and EtOAc. The organics were washed with brine, dried over MgSO₄, filtered, and concentrated on a rotary evaporator. The resulting crude material was dissolved in MeOH and purified using reverse phase preparatory HPLC (20-100% CH₃CN/H₂O). The fractions containing product were basified with 1 M K₂CO₃ and the CH₃CN removed on a rotary evaporator. The resulting aqueous mixture was extracted with EtOAc and the organic extract washed three times with 1 M K₂CO₃, once with brine, dried over MgSO₄, and filtered. The filtrate was concentrated on a rotary evaporator and dried under high vacuum to give 801 mg (36%) of (S)-2-(3-(benzyloxy)-2-methylpropyl)-4-(3,4-dichlorophenyl)-6-hydroxypyrimidine-5-carbonitrile as a yellow solid. ¹H NMR (300 MHz, DMSO-D6) δ ppm 0.90 (d, J=6.32 Hz, 3H) 2.24-2.43 (m, 2H) 2.55-2.64 (m, 1H) 3.24-3.40 (m, 3H) 4.45 (s, 2H) 7.21-7.37 (m, 5H) 7.71-7.80 (m, 2H) 7.95 (d, J=1.65 Hz, 1H). MS m/z calculated for (M+H)⁺ 428, found 428.

Step f: (S)-2-(3-(benzyloxy)-2-methylpropyl)-4-chloro-6-(3,4-dichlorophenyl)pyrimidine-5-carbonitrile. (S)-2-(3-(benzyloxy)-2-methylpropyl)-4-(3,4-dichlorophenyl)-6-hydroxypyrimidine-5-carbonitrile (795 mg, 1.86 mmol) was dissolved in 1,4-dioxane (12 mL) with stirring under N₂. POCl₃ (1.7 mL, 18.6 mmol) was added and the resulting mixture heated at 90° C. under a reflux condenser under N₂ for 3 hours. All volatiles were removed on a rotary evaporator at 70° C. for 1 hours. Dilute aqueous NaHCO₃ was added and the resulting mixture extracted with EtOAc. The organics were washed with brine and concentrated on a rotary evaporator. Flash chromatography (5-10% EtOAc/hexane) gave 480 mg (58%) of (S)-2-(3-(benzyloxy)-2-methylpropyl)-4-chloro-6-(3,4-dichlorophenyl)pyrimidine-5-carbonitrile as a light yellow oil. R_{f}=0.18 (10% EtOAc/hexane). ¹H NMR (300 MHz, DMSO-D6) δ ppm 0.97 (d, J=6.87 Hz, 3H) 2.87-2.97 (m, 1H) 3.01-3.11 (m, 1H) 3.28-3.48 (m, 3H) 4.31-4.43 (m, 2H) 7.14-7.21 (m, 2H) 7.23-7.34 (m, 3H) 7.84-7.90 (m, 1H) 7.91-7.96 (m, 1H) 8.08 (d, J=1.65 Hz, 1H).

Step g: (S)-5-amino-2-(3-(benzyloxy)-2-methylpropyl)-4-(3,4-dichlorophenyl)thieno[2,3-d]pyrimidine-6-carboxamide. 2-mercaptoacetamide (112 mg, 1.23 mmol) in ethanol (12 mL) was added to (S)-2-(3-(benzyloxy)-2-methylpropyl)-4-chloro-6-(3,4-dichlorophenyl)pyrimidine-5-carbonitrile (478 mg, 1.07 mmol) in a 100 mL flask with a stirbar. K₂CO₃ (170 mg, 1.23 mmol) was added and the resulting mixture stirred vigorously and heated at 50° C. under a reflux condenser under N₂ for 16 hours. Volatiles were removed on a rotary evaporator and the residue partitioned between H₂O and EtOAC. The organics were washed with brine and concentrated on a rotary evaporator. The residue was dissolved in DMSO/MeOH and purified using reverse phase preparatory HPLC (20-100% CH₃CN/H₂O). CH₃CN was removed on a rotary evaporator. The resulting aqueous mixture was extracted with EtOAc and the organic extract washed twice with 1 M K₂CO₃, once with brine, dried over MgSO₄, and filtered. The filtrate was concentrated on a rotary evaporator and dried under high vacuum to give 419 mg (78%) of (S)-5-amino-2-(3-(benzyloxy)-2-methylpropyl)-4-(3,4-dichlorophenyl)thieno[2,3-d]pyrimidine-6-carboxamide as a bright yellow solid. ¹H NMR (300 MHz, DMSO-D6) δ ppm 0.95 (d, J=6.87 Hz, 3H) 2.43-2.49 (m, 1H) 2.84 (dd, J=13.87, 7.55 Hz, 1H) 3.10 (dd, J=13.74, 6.59 Hz, 1H) 3.38 (d, J=6.32 Hz, 2H) 4.42 (s, 2H) 6.31 (s, 2H) 7.13-7.33 (m, 5H) 7.39 (s, 2H) 7.61 (dd, J=8.38, 2.06 Hz, 1H) 7.82 (d, J=8.24 Hz, 1H) 7.85 (d, J=1.92 Hz, 1H). MS m/z calculated for (M+H)⁺ 501, found 501.

Step h: (S)-5-amino-4-(3,4-dichlorophenyl)-2-(3-hydroxy-2-methylpropyl)thieno[2,3-d]pyrimidine-6-carboxamide. (S)-5-amino-2-(3-(benzyloxy)-2-methylpropyl)-4-(3,4-dichlorophenyl)thieno[2,3-d]pyrimidine-6-carboxamide (415 mg, 0.83 mmol) was dissolved in CH₂Cl₂ (40 mL) with stirring under N₂. The resulting solution was cooled to −65° C. and BBr₃ (4.14 mL of a 1 M solution in CH₂Cl₂, 4.14 mmol) was added dropwise. The resulting dark orange-brown mixture was stirred 40 minutes while being slowly warmed to −20° C. MeOH (10 mL) was added and the CH₂Cl₂ removed on a rotary evaporator. The residue was purified using reverse phase preparatory HPLC (20-100% CH₃CN/H₂O). CH₃CN was removed on a rotary evaporator. The resulting aqueous mixture was filtered. The collected solids were dissolved in hot EtOAc, washed three times with 1 M K₂CO₃, once with brine, dried over MgSO₄, filtered, and the filtrate concentrated on a rotary evaporator. The resulting residue was triturated with 50% Et₂O/hexane and dried under high vacuum to give 218 mg (64%) of (S)-5-amino-4-(3,4-dichlorophenyl)-2-(3-hydroxy-2-methylpropyl)thieno[2,3-d]pyrimidine-6-carboxamide as a yellow solid. ¹H NMR (400 MHz, DMSO-D6) δ ppm 0.85 (d, J=6.64 Hz, 3H) 2.18-2.30 (m, 1H) 2.69 (dd, J=13.67, 8.59 Hz, 1H) 3.12 (dd, J=13.67, 5.47 Hz, 1H) 3.29-3.37 (m, 2H) 4.57 (t, J=5.27 Hz, 1H) 6.32 (s, 2H) 7.38 (s, 2H) 7.66 (dd, J=8.20, 1.95 Hz, 1H) 7.83 (d, J=8.20 Hz, 1H) 7.93 (d, J=1.95 Hz, 1H). MS m/z calculated for (M+H)⁺ 411, found 411.

### 5.2.40 (R)-5-amino-4-(3,4-dichlorophenyl)-2-(3-hydroxy-2-methylpropyl)thieno[2,3-d]pyrimidine-6-carboxamide

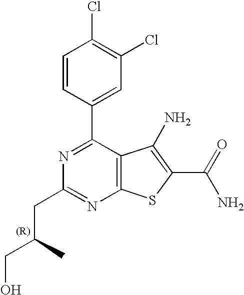

Step a: (S)-methyl3-(benzyloxy)-2-methylpropanoate. A stirred solution of (S)-methyl 3-hydroxy-2-methylpropanoate (9.60 g, 81.3 mmol) and benzyl 2,2,2-trichloroacetimidate (22.57 g, 89.4 mmol) in CH₂Cl₂ (14 mL) was cooled to 0° C. under N₂. Triflic acid (0.11 mL) in CH₂Cl₂ (1 mL) was added dropwise. After 10 minutes the reaction became a solid mass. After 1 hour, saturated aqueous NaHCO₃ was added and the mixture extracted with Et₂O. The organics were dried over MgSO₄, filtered and concentrated on a rotary evaporator. Hexane was added and the resulting solids collected by vacuum filtration. The filtrate was concentrated on a rotary evaporator and purified using flash chromatography (5-10% EtOAc/hexane) to give 11.82 g (70%) of (S)-methyl3-(benzyloxy)-2-methylpropanoate as a colorless oil. R_{f}=0.20 (10% EtOAc/hexane). ¹H NMR (300 MHz, DMSO-D6) δ ppm 1.08 (d, J=7.14 Hz, 3H) 2.75 (ddd, J=14.01, 12.64, 7.14 Hz, 1H) 3.44-3.59 (m, 2H) 3.60 (s, 3H) 4.46 (s, 2H) 7.23-7.40 (m, 5H).

Step b: (R)-3-(benzyloxy)-2-methylpropan-1-ol. A vigorously stirred suspension of LiAlH₄ (4.52 g, 119 mmol) in THF (40 mL) was cooled to −75° C. under N₂. (S)-methyl3-(benzyloxy)-2-methylpropanoate (11.82 g, (56.8 mmol) in THF (10 mL) was added dropwise via pipette. The resulting reaction mixture was stirred 30 minutes at −75° C. Saturated aqueous Na₂SO₄ was added dropwise via addition funnel until all LiAlH₄ was quenched (salts turn white). The resulting mixture was filtered and the filter cake washed twice with Et₂O. The filtrate was concentrated on a rotary evaporator and purified using flash chromatography (10-30% EtOAc/hexane) to give 7.73 g (76%) of (R)-3-(benzyloxy)-2-methylpropan-1-ol as a colorless oil. R_{f}=0.23 (30% EtOAc/hexane). ¹H NMR (300 MHz, DMSO-D6) δ ppm 0.86 (d, J=6.87 Hz, 3H) 1.74-1.88 (m, 1H) 3.20-3.45 (m, 4H) 4.38-4.47 (m, 3H) 7.22-7.40 (m, 5H). MS m/z calculated for (M+H)⁺ 181, found 181.

Step c: (R)-4-(benzyloxy)-3-methylbutanenitrile. (R)-3-(benzyloxy)-2-methylpropan-1-ol (1.00 g, 5.55 mmol) and triethylamine (1.55 mL, 11.1 mmol) were dissolved in CH₂Cl₂ (8 mL) with stirring under N₂ and cooled to 0° C. Methanesulfonyl chloride (0.47 mL, 6.10 mmol) in CH₂Cl₂ (1 mL) was added dropwise. The resulting mixture (cloudy light yellow) was stirred 40 minutes at 0° C. and then all volatiles were removed on a rotary evaporator. DMSO (8 mL), NaCN (0.82 g, 16.6 mmol), and dimethylaminopyridine (0.01 g, 0.07 mmol) were added and the resulting mixture stirred vigorously and heated at 50° C. for 24 hours. H₂O was added and the mixture extracted with Et₂O. The organics were washed twice with H₂O, once with brine, and concentrated on a rotary evaporator. Flash chromatography (10-20% EtOAc/hexane) gave 778 mg (74%) of (R)-4-(benzyloxy)-3-methylbutanenitrile as a colorless oil. R_{f}=0.39 (30% EtOAc/hexane). ¹H NMR (300 MHz, DMSO-D6) δ ppm 0.98 (d, J=6.59 Hz, 3H) 2.01-2.19 (m, 1H) 2.41-2.64 (m, 2H) 3.23-3.41 (m, 2H) 4.48 (s, 2H) 7.20-7.44 (m, 5H). MS m/z calculated for (M+H)⁺ 190, found 190.

Step d: (R)-4-(benzyloxy)-3-methylbutanimidamide. (R)-4-(benzyloxy)-3-methylbutanenitrile (0.773 g, 4.08 mmol) was dissolved in ethanol (10 mL) with stirring and cooled to 0° C. HCl gas was bubbled in for 30 minutes and then the flask was capped and stirred at 0° C. for 4 hours. All volatiles were removed on a rotary evaporator for 30 minutes. Meanwhile NH₃ gas was bubbled into ethanol (15 mL) at 0° C. for 20 minutes. The resulting NH₃/ethanol solution was poured onto the above-prepared crude imidate at 0° C. The resulting mixture was stirred vigorously while being slowly warmed to room temperature for 16 hours. All volatiles were removed on a rotary evaporator. Toluene (10 mL) was added and then removed on a rotary evaporator at 65° C. for 30 minutes. This material was used in the next step without purification. MS m/z calculated for (M+H)⁺ 207, found 207.

Step e: (R)-2-(3-(benzyloxy)-2-methylpropyl)-4-(3,4-dichlorophenyl)-6-hydroxypyrimidine-5-carbonitrile. 3,4-dichlorobenzaldehyde (715 mg, 4.08 mmol), ethyl cyanoacetate (462 mg, 4.08 mmol), and piperidine (1 drop) were refluxed in toluene (7 mL) with azeotropic removal of H₂O under N₂ for 30 minutes. The stirbar was removed and the volatiles removed on a rotary evaporator. To the resulting material was added sequentially: a stirbar, (R)-4-(benzyloxy)-3-methylbutanimidamide (4.08 mmol crude), K₂CO₃ (1.13 g, 8.17 mmol), and ethanol (15 mL). The resulting mixture was stirred vigorously and heated at 75° C. under a reflux condenser under N₂ for 4.5 hours. Heating was discontinued and the resulting mixture partitioned between ice water and EtOAc. The organics were washed with brine, dried over MgSO₄, filtered, and concentrated on a rotary evaporator. The resulting crude material was dissolved in MeOH and purified using reverse phase preparatory HPLC (20-100% CH₃CN/H₂O). The fractions containing product were basified with 1 M K₂CO₃ and the CH₃CN removed on a rotary evaporator. The resulting aqueous mixture was extracted with EtOAc and the organic extract washed three times with 1 M K₂CO₃, once with brine, dried over MgSO₄, and filtered. The filtrate was concentrated on a rotary evaporator and dried under high vacuum to give 578 mg (33%) of (R)-2-(3-(benzyloxy)-2-methylpropyl)-4-(3,4-dichlorophenyl)-6-hydroxypyrimidine-5-carbonitrile as a yellow solid. ¹H NMR (300 MHz, DMSO-D6) δ ppm 0.90 (d, J=6.32 Hz, 3H) 2.24-2.43 (m, 2H) 2.55-2.64 (m, 1H) 3.24-3.40 (m, 3H) 4.45 (s, 2H) 7.21-7.37 (m, 5H) 7.71-7.80 (m, 2H) 7.95 (d, J=1.65 Hz, 1H). MS m/z calculated for (M+H)⁺ 428, found 428.

Step f: (R)-2-(3-(benzyloxy)-2-methylpropyl)-4-chloro-6-(3,4-dichlorophenyl)pyrimidine-5-carbonitrile. (R)-2-(3-(benzyloxy)-2-methylpropyl)-4-(3,4-dichlorophenyl)-6-hydroxypyrimidine-5-carbonitrile (573 mg, 1.34 mmol) was dissolved in 1,4-dioxane (12 mL) with stirring under N₂. POCl₃ (1.7 mL, 18.6 mmol) was added and the resulting mixture heated at 90° C. under a reflux condenser under N₂ for 3 hours. All volatiles were removed on a rotary evaporator at 70° C. for 1 hour. Dilute aqueous NaHCO₃ was added and the resulting mixture extracted with EtOAc. The organics were washed with brine and concentrated on a rotary evaporator. Flash chromatography (5-10% EtOAc/hexane) gave 292 mg (49%) of (R)-2-(3-(benzyloxy)-2-methylpropyl)-4-chloro-6-(3,4-dichlorophenyl)pyrimidine-5-carbonitrile as a light yellow oil. R_{f}=0.18 (10% EtOAc/hexane). ¹H NMR (300 MHz, DMSO-D6) δ ppm 0.97 (d, J=6.87 Hz, 3H) 2.87-2.97 (m, 1H) 3.01-3.11 (m, 1H) 3.28-3.48 (m, 3H) 4.31-4.43 (m, 2H) 7.14-7.21 (m, 2H) 7.23-7.34 (m, 3H) 7.84-7.90 (m, 1H) 7.91-7.96 (m, 1H) 8.08 (d, J=1.65 Hz, 1H).

Step g: (R)-5-amino-2-(3-(benzyloxy)-2-methylpropyl)-4-(3,4-dichlorophenyl)thieno[2,3-d]pyrimidine-6-carboxamide. 2-mercaptoacetamide (60 mg, 0.66 mmol) in ethanol (7 mL) was added to (R)-2-(3-(benzyloxy)-2-methylpropyl)-4-chloro-6-(3,4-dichlorophenyl)pyrimidine-5-carbonitrile (282 mg, 0.63 mmol) in a 100 mL flask with a stirbar. K₂CO₃ (92 mg, 0.66 mmol) was added and the resulting mixture stirred vigorously and heated at 50° C. under a reflux condenser under N₂ for 16 hours. Volatiles were removed on a rotary evaporator and the residue partitioned between H₂O and EtOAC. The organics were washed with brine and concentrated on a rotary evaporator. The residue was dissolved in DMSO/MeOH and purified using reverse phase preparatory HPLC (20-100% CH₃CN/H₂O). CH₃CN was removed on a rotary evaporator. The resulting aqueous mixture was extracted with EtOAc and the organic extract washed twice with 1 M K₂CO₃, once with brine, dried over MgSO₄, and filtered. The filtrate was concentrated on a rotary evaporator and dried under high vacuum to give 151 mg (48%) of (R)-5-amino-2-(3-(benzyloxy)-2-methylpropyl)-4-(3,4-dichlorophenyl)thieno[2,3-d]pyrimidine-6-carboxamide as a yellow solid. ¹H NMR (300 MHz, DMSO-D6) δ ppm 0.95 (d, J=6.87 Hz, 3H) 2.43-2.49 (m, 1H) 2.84 (dd, J=13.87, 7.55 Hz, 1H) 3.10 (dd, J=13.74, 6.59 Hz, 1H) 3.38 (d, J=6.32 Hz, 2H) 4.42 (s, 2H) 6.31 (s, 2H) 7.13-7.33 (m, 5H) 7.39 (s, 2H) 7.61 (dd, J=8.38, 2.06 Hz, 1H) 7.82 (d, J=8.24 Hz, 1H) 7.85 (d, J=1.92 Hz, 1H). MS m/z calculated for (M+H)⁺ 501, found 501.

Step h: (R)-5-amino-4-(3,4-dichlorophenyl)-2-(3-hydroxy-2-methylpropyl)thieno[2,3-d]pyrimidine-6-carboxamide. (R)-5-amino-2-(3-(benzyloxy)-2-methylpropyl)-4-(3,4-dichlorophenyl)thieno[2,3-d]pyrimidine-6-carboxamide (135 mg, 0.27 mmol) was dissolved in CH₂Cl₂ (18 mL) with stirring under N₂. The resulting solution was cooled to −65° C. and BBr₃ (1.35 mL of a 1 M solution in CH₂Cl₂, 1.35 mmol) was added dropwise. The resulting dark orange-brown mixture was stirred 40 minutes while being slowly warmed to −20° C. MeOH (8 mL) was added and the CH₂Cl₂ removed on a rotary evaporator. The residue was purified using reverse phase preparatory HPLC (20-100% CH₃CN/H₂O). CH₃CN was removed on a rotary evaporator. The resulting aqueous mixture was filtered. The collected solids were dissolved in hot EtOAc, washed three times with 1 M K₂CO₃, once with brine, dried over MgSO₄, filtered, and the filtrate concentrated on a rotary evaporator. The resulting residue was triturated with 50% Et₂O/hexane and dried under high vacuum to give 65 mg (59%) of (R)-5-amino-4-(3,4-dichlorophenyl)-2-(3-hydroxy-2-methylpropyl)thieno[2,3-d]pyrimidine-6-carboxamide as a yellow solid. ¹H NMR (400 MHz, DMSO-D6) δ ppm 0.85 (d, J=6.64 Hz, 3H) 2.18-2.30 (m, 1H) 2.69 (dd, J=13.67, 8.59 Hz, 1H) 3.12 (dd, J=13.67, 5.47 Hz, 1H) 3.29-3.37 (m, 2H) 4.57 (t, J=5.27 Hz, 1H) 6.32 (s, 2H) 7.38 (s, 2H) 7.66 (dd, J=8.20, 1.95 Hz, 1H) 7.83 (d, J=8.20 Hz, 1H) 7.93 (d, J=1.95 Hz, 1H). MS m/z calculated for (M+H)⁺ 411, found 411.

### 5.2.41 5-Amino-4-(3-methoxy-5-pyridyl)-2-cyclopropylamino-thieno[2,3-d]pyrimidine-6-carboxamide

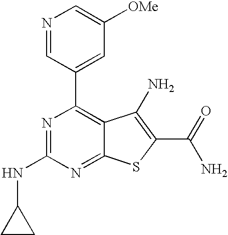

Step a: 5-Cyano-4-(3-methoxy-5-pyridyl)-2-cyclopropylamino-6-oxopyrimidine. 3-Methoxy-5-formylpyridine (2.01 g, 14.8 mmol), ethylcyanoacetate (1.67 g, 14.8 mmol), N-cyclopropylguanidine.HCl (1.99 g, 14.8 mmol), and potassium carbonate (2.65 g, 19.2 mmol) was stirred in ethanol (70 mL) at 75° C. for 18 hours. The reaction mixture was partitioned between ethyl acetate and water. The organic layer was separated and concentrated to give a residue which was purified using flash chromatography (100% EtOAc) to give 5-cyano-4-(3-methoxy-5-pyridyl)-2-cyclopropylamino-6-oxopyrimidine (1.46 g, 35%). MS m/z calculated for (M+H)⁺ 284, found 284.

Step b: 6-Chloro-5-cyano-4-(3-methoxy-5-pyridyl)-2-cyclopropylaminopyrimidine. 5-Cyano-4-(3-methoxy-5-pyridyl)-2-cyclopropylamino-6-oxopyrimidine (1.46 g, 5.15 mmol) was stirred in POCl₃ (10 mL). The mixture was heated to 90° C. for 2 hours then volatiles were removed and the residue was partitioned between ethyl acetate and sat. NaHCO₃. The organic layer was separated and concentrated to give a residue which was recrystallized from ethanol to give 6-chloro-5-cyano-4-(3-methoxy-5-pyridyl)-2-cyclopropylaminopyrimidine (0.25 g, 16%) as a yellow solid. MS m/z calculated for (M+H)⁺ 302, found 302.

Step c: 5-Amino-4-(3-methoxy-5-pyridyl)-2-cyclopropylamino-thieno[2,3-d]pyrimidine-6-carboxamide. 6-Chloro-5-cyano-4-(3-methoxy-5-pyridyl)-2-cyclopropylaminopyrimidine (0.25 g, 0.83 mmol), 2-mercaptoacetamide (0.075 g, 0.83 mmol), and sodium carbonate (0.105 g, 0.99 mmol) was stirred in ethanol (10 mL) at 60° C. for 4 hours. The reaction mixture was partitioned between ethyl acetate and water. The organic layer was separated and concentrated to give a residue. Excess sodium ethoxide in ethanol was added and the mixture was heated at 75° C. for 2 hours. The reaction mixture was partitioned between ethyl acetate and water. The organic layer was separated and concentrated to give a residue which was purified by prep HPLC to give the title compound 5-amino-4-(3-methoxy-5-pyridyl)-2-cyclopropylamino-thieno[2,3-d]pyrimidine-6-carboxamide (0.12 g, 42%) as a yellow solid. ¹H NMR (DMSO-d₆): δ 8.55 (m, 2H), 8.05 (s, 1H), 7.60 (s, 1H), 7.04 (s, 2H), 6.03 (s, 2H), 3.88 (s, 3H), 2.82 (m, 1H), 0.72 (t, 2H), 0.53 (t, 2H). MS m/z/Z calculated for (M+H)⁺ 357, found 357.

### 5.2.42 5-Amino-4-(4-methylphenyl)-2-cyclopropylamino-thieno[2,3-d]pyrimidine-6-carboxamide

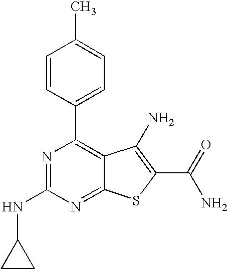

Step a: 5-Cyano-4-(4-methylphenyl)-2-cyclopropylamino-6-oxopyrimidine. Para-Tolualdehyde (1.28 g, 14.8 mmol), ethylcyanoacetate (1.67 g, 14.8 mmol), N-cyclopropylguanidine.HCl (1.99 g, 14.8 mmol), and potassium carbonate (2.65 g, 19.2 mmol) were stirred in ethanol (50 mL) at 75° C. for 18 hours. The reaction mixture was partitioned between ethyl acetate and water. The organic layer was separated and concentrated to give 5-cyano-4-(4-methylphenyl)-2-cyclopropylamino-6-oxopyrimidine (0.65 g, 17%). MS m/z calculated for (M+H)⁺ 254, found 254.

Step b: 6-Chloro-5-cyano-4-(4-methylphenyl)-2-cyclopropylaminopyrimidine. 5-Cyano-4-(4-methylphenyl)-2-cyclopropylamino-6-oxopyrimidine (0.64 g, 2.52 mmol) was stirred in POCl₃ (10 mL). The mixture was heated to 90° C. for 2 hours then volatiles were removed and the residue was partitioned between ethyl acetate and sat. NaHCO₃. The organic layer was separated and concentrated to give a residue which was recrystallized from ethanol to give 6-chloro-5-cyano-4-(4-methylphenyl)-2-cyclopropylaminopyrimidine (0.40 g, 58%) as a yellow solid. MS m/z calculated for (M+H)⁺ 272, found 272.

Step c: 5-Amino-4-(4-methylphenyl)-2-cyclopropylamino-thieno[2,3-d]pyrimidine-6-carboxamide. 6-Chloro-5-cyano-4-(4-methylphenyl)-2-cyclopropylaminopyrimidine (0.40 g, 1.47 mmol), 2-mercaptoacetamide (0.134 g, 1.74 mmol), and sodium carbonate (0.187 g, 1.77 mmol) was stirred in ethanol (10 mL) at 60° C. for 4 hours. The reaction mixture was partitioned between ethyl acetate and water. The organic layer was separated and concentrated to give a residue. Excess sodium ethoxide in ethanol was added and the mixture was heated at 75° C. for 2 hours. The reaction mixture was partitioned between ethyl acetate and water. The organic layer was separated and concentrated to give a residue which was purified by prep HPLC to give the title compound 5-amino-4-(4-methylphenyl)-2-cyclopropylamino-thieno[2,3-d]pyrimidine-6-carboxamide (0.16 g, 35%) as a yellow solid. ¹H NMR (DMSO-d₆): δ 7.95 (s, 1H), 7.37 (m, 4H), 6.98 (s, 2H), 6.00 (s, 2H), 2.82 (m, 1H), 2.51 (s, 3H), 0.72 (t, 2H), 0.52 (t, 2H). MS m/z calculated for (M+H)⁺ 327, found 327.

### 5.2.43 5-Amino-4-(4-chlorophenyl)-2-cyclopropylamino-thieno[2,3-d]pyrimidine-6-carboxamide

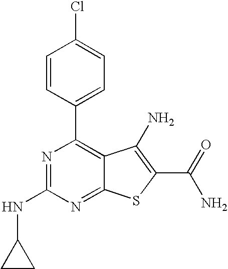

Step a: 5-Cyano-4-(4-chlorophenyl)-2-cyclopropylamino-6-oxopyrimidine. 4-Chlorobenzaldehyde (2.08 g, 14.8 mmol), ethylcyanoacetate (1.67 g, 14.8 mmol), N-cyclopropylguanidine.HCl (1.99 g, 14.8 mmol), and potassium carbonate (2.65 g, 19.2 mmol) was stirred in ethanol (75 mL) at 75° C. for 22 hours. The reaction mixture was partitioned between ethyl acetate and water. The organic layer was separated and concentrated to give 5-cyano-4-(4-chlorophenyl)-2-cyclopropylamino-6-oxopyrimidine (1.40 g, 33%). MS m/z calculated for (M+H)⁺ 274, found 274.

Step b: 6-Chloro-5-cyano-4-(4-methylphenyl)-2-cyclopropylaminopyrimidine. 5-Cyano-4-(4-chlorophenyl)-2-cyclopropylamino-6-oxopyrimidine (1.30 g, 4.76 mmol) was stirred in POCl₃ (25 mL). The mixture was heated to 90° C. for 2 hours. then volatiles were removed and the residue was partitioned between ethyl acetate and sat. NaHCO₃. The organic layer was separated and concentrated to give 6-chloro-5-cyano-4-(4-chlorophenyl)-2-cyclopropylaminopyrimidine (0.99 g, 72%) as a yellow solid. MS m/z calculated for (M+H)⁺ 292, found 292.

Step c: 5-Amino-4-(4-chlorophenyl)-2-cyclopropylamino-thieno[2,3-d]pyrimidine-6-carboxamide. 6-Chloro-5-cyano-4-(4-chlorophenyl)-2-cyclopropylaminopyrimidine (0.85 g, 2.92 mmol), 2-mercaptoacetamide (0.26 g, 2.92 mmol), and sodium carbonate (0.37 g, 3.50 mmol) was stirred in ethanol (30 mL) at 60° C. for 4 hours. The reaction mixture was partitioned between ethyl acetate and water. The organic layer was separated and concentrated to give a residue. Excess sodium ethoxide in ethanol was added and the mixture was heated at 75° C. for 2 hours. The reaction mixture was partitioned between ethyl acetate and water. The organic layer was separated and concentrated to give a residue which was purified using preparative HPLC to give the title compound 5-amino-4-(4-chlorophenyl)-2-cyclopropylamino-thieno[2,3-d]pyrimidine-6-carboxamide (0.31 g, 30%) as a yellow solid. ¹H NMR (DMSO-d₆): δ 7.97 (s, 1H), 7.60 (s, 4H), 6.99 (s, 2H), 6.03 (s, 2H), 2.80 (m, 1H), 0.70 (t, 2H), 0.51 (t, 2H). MS m/z calculated for (M+H)⁺ 360, found 360.

### 5.2.44 5-Amino-4-(3-pyridyl-4-benzyloxy)-2-cyclopropylamino-thieno[2,3-d]pyrimidine-6-carboxamide

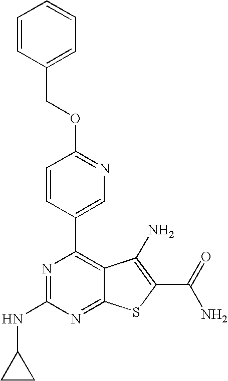

Step a: 3-pyridyl-4-benzyloxy-aldehyde. 3-Bromopyridone (5.00 g, 28.7 mmol), silver carbonate (5.30 g, 19.2 mmol) and benzyl bromide (5.89 g, 34.4 mmol) was stirred in benzene (50 mL) at 50° C. for 18 hours. The reaction mixture was filtered through Celite and partitioned between ethyl acetate and water. The organic layer was separated and concentrated to give a residue which was treated with n-butyl lithium and DMF by literature method to give 3-pyridyl-4-benzyloxy-aldehyde (3.00 g, 49%). MS m/z calculated for (M+H)⁺ 213, found 213.

Step b: 5-Cyano-4-(3-pyridyl-4-benzyloxy)-2-cyclopropylamino-6-oxopyrimidine. 3-pyridyl-4-benzyloxy-aldehyde (3.00 g, 14.1 mmol), ethylcyanoacetate (1.59 g, 14.1 mmol), N-cyclopropylguanidine.HCl (1.91 g, 14.1 mmol), and potassium carbonate (1.95 g, 14.1 mmol) was stirred in ethanol (70 mL) at 75° C. for 18 hours. The reaction mixture was partitioned between ethyl acetate and water. The organic layer was separated and concentrated to give 5-cyano-4-(3-pyridyl-4-benzyloxy)-2-cyclopropylamino-6-oxopyrimidine (1.40 g, 30%). MS m/z calculated for (M+H)⁺ 360, found 360.

Step c: 6-Chloro-5-cyano-4-(3-pyridyl-4-benzyloxy)-2-cyclopropylaminopyrimidine. 5-Cyano-4-(3-pyridyl-4-benzyloxy)-2-cyclopropylamino-6-oxopyrimidine (0.70 g, 1.95 mmol) was stirred in dioxane (10 mL) and POCl₃ (10 mL). The mixture was heated to 90° C. for 1.5 hours then volatiles were removed and the residue was partitioned between ethyl acetate and sat. NaHCO₃. The organic layer was separated and concentrated to give, after flash chromatography, 6-chloro-5-cyano-4-(3-pyridyl-4-benzyloxy)-2-cyclopropylaminopyrimidine (0.20 g, 27%) as a yellow solid. MS m/z calculated for (M+H)⁺ 378, found 378.

Step d: 5-Amino-4-(3-pyridyl-4-benzyloxy)-2-cyclopropylamino-thieno[2,3-d]pyrimidine-6-carboxamide. 6-Chloro-5-cyano-4-(3-pyridyl-4-benzyloxy)-2-cyclopropylaminopyrimidine (0.20 g, 0.53 mmol), 2-mercaptoacetamide (0.048 g, 0.53 mmol) was stirred in ethanol (10 mL) followed by the addition of at excess sodium ethoxide in ethanol. The mixture was heated at 75° C. for 2 hours. The reaction mixture was partitioned between ethyl acetate and water. The organic layer was separated and concentrated to give a residue which was purified using preparative HPLC to give the title compound 5-amino-4-(3-pyridyl-4-benzyloxy)-2-cyclopropylamino-thieno[2,3-d]pyrimidine-6-carboxamide (0.13 g, 55%) as a yellow solid. ¹H NMR (DMSO-d₆): δ 8.40 (s, 1H), 7.97 (m, 2H), 7.30-7.50 (m, 5H), 7.00-7.03 (m, 3H), 6.20 (s, 2H), 5.43 (s, 2H), 2.90 (m, 1H), 0.72 (t, 2H), 0.53 (t, 2H). MS m/z calculated for (M+H)⁺ 433, found 433.

### 5.2.45 5-Amino-4-(3-pyridyl-4-chloro)-2-cyclopropylamino-thieno[2,3-d]pyrimidine-6-carboxamide

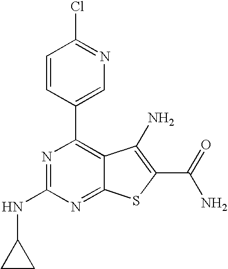

Step a: 6-Chloro-5-cyano-4-(3-pyridyl-4-chloro)-2-cyclopropylaminopyrimidine. 5-Cyano-4-(3-pyridyl-4-benzyloxy)-2-cyclopropylamino-6-oxopyrimidine (0.70 g, 1.95 mmol) was stirred in dioxane (10 mL) and POCl₃ (10 mL). The mixture was heated to 90° C. for 1.5 hours then volatiles were removed and the residue was partitioned between ethyl acetate and sat. NaHCO₃. The organic layer was separated and concentrated to give, after flash chromatography, 6-chloro-5-cyano-4-(3-pyridyl-4-chloro)-2-cyclopropyl-aminopyrimidine (0.20 g, 34%) as a yellow solid. MS m/z calculated for (M+H)⁺ 306, found 306.

Step b: 5-Amino-4-(3-pyridyl-4-chloro)-2-cyclopropylamino-thieno[2,3-d]pyrimidine-6-carboxamide. 6-Chloro-5-cyano-4-(3-pyridyl-4-chloro)-2-cyclopropylaminopyrimidine (0.20 g, 0.65 mmol), 2-mercaptoacetamide (0.059 g, 0.65 mmol) was stirred in ethanol (10 mL) followed by the addition of at excess sodium ethoxide in ethanol. The mixture was heated at 75° C. for 2 hours. The reaction mixture was partitioned between ethyl acetate and water. The organic layer was separated and concentrated to give a residue which was purified by prep HPLC to give the title compound 5-amino-4-(3-pyridyl-4-chloro)-2-cyclopropylamino-thieno[2,3-d]pyrimidine-6-carboxamide (0.085 g, 36%) as a yellow solid. ¹H NMR (DMSO-d₆): δ 8.60 (s, 1H), 8.05 (m, 2H), 7.68 (d, 2H), 7.05 (s, 2H), 6.20 (s, 2H), 2.95 (m, 1H), 0.71 (t, 2H), 0.51 (t, 2H). MS m/z calculated for (M+H)⁺ 361, found 361.

### 5.2.46 5-Amino-4-(3-pyridyl-4-hydroxy)-2-cyclopropylamino-thieno[2,3-d]pyrimidine-6-carboxamide

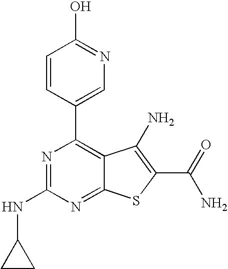

5-amino-4-(3-pyridyl-4-chloro)-2-cyclopropylamino-thieno[2,3-d]pyrimidine-6-carboxamide (0.045 g, 0.12 mmol) was added to a microwave tube along with trifluoroacetic acid (2 mL) and heated to 70° C. for 15 minutes. HPLC purification gave 6-chloro-5-cyano-4-(3-pyridyl-4-chloro)-2-cyclopropylaminopyrimidine (0.008 g, 19%) as a yellow solid. ¹H NMR (DMSO-d₆): δ 11.9 (s, 1H), 8.73 (s, 1H), 8.50 (s, 1H), 7.20-8.10 (m, 5H), 6.70 (d, 1H), 2.75 (m, 1H), 0.75 (t, 2H), 0.55 (t, 2H). MS m/z calculated for (M+H)⁺ 343, found 343.

### 5.2.47 5-Amino-4-(3-chloro-5-pyridyl)-2-cyclopropylamino-thieno[2,3-d]pyrimidine-6-carboxamide

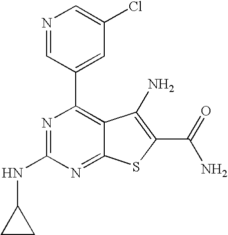

Step a: 5-Cyano-4-(3-chloro-5-pyridyl)-2-cyclopropylamino-6-oxopyrimidine. 3-Chloro-5-formylpyridine (0.50 g, 3.57 mmol), ethylcyanoacetate (0.40 g, 3.57 mmol), N-cyclopropylguanidine.HCl (0.48 g, 3.57 mmol), and potassium carbonate (0.54 g, 3.92 mmol) was stirred in ethanol (20 mL) at 75° C. for 18 hours. The reaction mixture was partitioned between ethyl acetate and water. The organic layer was separated and concentrated to give a residue which was purified using flash chromatography (100% EtOAc) to give 5-cyano-4-(3-chloro-5-pyridyl)-2-cyclopropylamino-6-oxopyrimidine (0.26 g, 26%). MS m/z calculated for (M+H)⁺ 287, found 287.

Step b: 6-Chloro-5-cyano-4-(3-chloro-5-pyridyl)-2-cyclopropylaminopyrimidine. 5-Cyano-4-(3-chloro-5-pyridyl)-2-cyclopropylamino-6-oxopyrimidine (0.26 g, 0.90 mmol) was stirred in POCl₃ (5 mL). The mixture was heated to 90° C. for 1 hour then volatiles were removed and the residue was partitioned between ethyl acetate and sat. NaHCO₃. The organic layer was separated and concentrated to give 6-chloro-5-cyano-4-(3-chloro-5-pyridyl)-2-cyclopropylaminopyrimidine (0.19 g, 70%) as a yellow solid. MS m/z calculated for (M+H)⁺ 305, found 305.

Step c: 5-Amino-4-(3-chloro-5-pyridyl)-2-cyclopropylamino-thieno[2,3-d]pyrimidine-6-carboxamide. 6-Chloro-5-cyano-4-(3-chloro-5-pyridyl)-2-cyclopropylaminopyrimidine (0.19 g, 0.62 mmol), 2-mercaptoacetamide (0.058 g, 0.63 mmol),) was stirred in ethanol (10 mL) followed by the addition of at excess sodium ethoxide in ethanol. The mixture was heated at 75° C. for 2 hours. The reaction mixture was partitioned between ethyl acetate and water. The organic layer was separated and concentrated to give a residue which was purified by prep HPLC to give the title compound 5-amino-4-(3-chloro-5-pyridyl)-2-cyclopropylamino-thieno[2,3-d]pyrimidine-6-carboxamide (0.029 g, 13%) as a yellow solid. ¹H NMR (DMSO-d₆): δ 8.73-8.78 (m, 2H), 8.14 (s, 1H), 8.03 (s, 1H), 7.06 (s, 2H), 6.19 (s, 2H), 2.82 (m, 1H), 0.71 (t, 2H), 0.52 (t, 2H). MS m/z calculated for (M+H)⁺ 360, found 360.

### 5.2.48 5-Amino-4-(3-ethoxy-5-pyridyl)-2-(2-amino-2-methyl-1-propanol)-thieno[2,3-d]pyrimidine-6-carboxamide

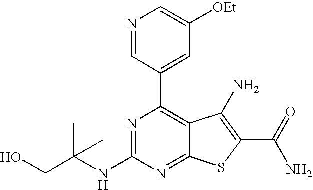

Step a: 3-Ethoxy-5-formylpyridine. 3-Bromo-5-ethoxypyridine (6.50 g, 32.1 mmol) was dissolved in tetrahydrofuran (40 mL) and cooled to 0° C. Isopropylmagnesiumchloride (17 mL, 2.0 M in THF) was syringed in and the reaction mixture was stirred for 2 hours at room temperature. N,N-dimethylformamide (5 mL) in tetrahydrofuran (12 mL) was added and stirring was continued for an additional hour. The solution was quenched with 2 N HCl to pH of 3 then partitioned between ethyl acetate and water. The organic layer was separated and concentrated to give a residue which was purified using flash chromatography to give 3-ethoxy-5-formylpyridine (3.00 g, 62%) as a yellow oil. ¹H NMR (DMSO-d₆): δ 10.1 (s, 1H), 8.65 (d, 1H), 8.54 (d, 1H), 7.59 (d, 1H), 4.15 (q, 2H), 1.47 (t, 3H). MS m/z/z calculated for (M+H)⁺ 152, found 152.

Step b: 5-Cyano-4-(3-ethoxy-5-pyridyl)-2-methylthio-6-oxopyrimidine. 3-Ethoxy-5-formylpyridine (1.50 g, 9.93 mmol), ethylcyanoacetate (1.12 g, 9.93 mmol), S-methylisothiouronium sulfate (1.38 g, 4.95 mmol), and potassium carbonate (1.65 g, 11.9 mmol) was stirred in ethanol (50 mL) at 75° C. for 18 hours. The reaction mixture was cooled to 0° C. and the resulting solid was filtered, washed with ethanol, then transferred to water (200 mL). The solution was acidified to pH 3 with 2 N HCl and the solid produced was filtered and dried to give 5-cyano-4-(3-ethoxy-5-pyridyl)-2-methylthio-6-oxopyrimidine (1.22 g, 43%) as a white solid. MS m/z calculated for (M+H)⁺ 289, found 289.

Step c: 6-Chloro-5-cyano-4-(3-ethoxy-5-pyridyl)-2-methylthiopyrimidine. 5-Cyano-4-(3-ethoxy-5-pyridyl)-2-methylthio-6-oxopyrimidine (1.20 g, 4.16 mmol) was stirred in dioxane (20 mL) and POCl₃ (20 mL). The mixture was heated to 90° C. for 3 hours then volatiles were removed and the residue was partitioned between ethyl acetate and sat. NaHCO₃. The organic layer was separated and concentrated to give 6-chloro-5-cyano-4-(3-ethoxy-5-pyridyl)-2-methylthiopyrimidine (1.20 g, 94%) as a yellow solid. MS m/z calculated for (M+H)⁺ 307, found 307.

Step d: 5-Amino-4-(3-ethoxy-5-pyridyl)-2-methylthio-thieno[2,3-d]pyrimidine-6-carboxamide. 6-chloro-5-cyano-4-(3-ethoxy-5-pyridyl)-2-methylthiopyrimidine (1.15 g, 3.76 mmol), 2-mercaptoacetamide (0.36 g, 3.95 mmol), and sodium carbonate (0.42 g, 3.94 mmol) was stirred in ethanol (30 mL) at 60° C. for 3 hours. The reaction mixture was partitioned between ethyl acetate and water. The organic layer was separated and concentrated to give a residue. Excess sodium ethoxide in ethanol was added and the mixture was heated at 75° C. for 2 hours. The reaction mixture was partitioned between ethyl acetate and water. The organic layer was separated and concentrated to give 5-Amino-4-(3-ethoxy-5-pyridyl)-2-methylthio-thieno[2,3-d]pyrimidine-6-carboxamide (0.61 g, 45%) as a yellow solid. MS m/z calculated for (M+H)⁺ 362, found 362.

Step e: 5-Amino-4-(3-ethoxy-5-pyridyl)-2-methylsulfoxide-thieno[2,3-d]pyrimidine-6-carboxamide. 5-Amino-4-(3-ethoxy-5-pyridyl)-2-methylthio-thieno[2,3-d]pyrimidine-6-carboxamide (0.57 g, 1.57 mmol) was stirred in chloroform (90 mL) at −5° C. mCPBA (0.49 g, 2.83 mmol) was slowly added and stirring was continued for 40 minutes at −5° C. The reaction mixture was partitioned between chloroform and aqueous sodium bisulfite and sodium bicarbonate. The organic layer was separated and concentrated to give 5-Amino-4-(3-ethoxy-5-pyridyl)-2-methylsulfoxide-thieno[2,3-d]pyrimidine-6-carboxamide (0.43 g, 72%) as a yellow solid. MS m/z calculated for (M+H)⁺ 378, found 378.

Step f: 5-Amino-4-(3-ethoxy-5-pyridyl)-2-(2-amino-2-methyl-1-propanol)-thieno[2,3-d]pyrimidine-6-carboxamide. 5-Amino-4-(3-ethoxy-5-pyridyl)-2-methylsulfoxide-thieno[2,3-d]pyrimidine-6-carboxamide (0.41 g, 1.08 mmol), 2-amino-2-methyl-1-propanol (0.15 g, 1.63 mmol) and diisopropylethylamine (0.21, 1.63 mmol) was stirred in DMF (50 mL) at 90° C. for 3 hours. Volatiles were removed and the residue was purified using preparative HPLC to give the title compound 5-Amino-4-(3-ethoxy-5-pyridyl)-2-(2-amino-2-methyl-1-propanol)-thieno[2,3-d]pyrimidine-6-carboxamide (0.045 g, 10%) as a yellow solid. ¹H NMR (DMSO-d₆): δ 8.42 (d, 1H), 8.35 (s, 1H), 7.56 (m, 1H), 7.12 (s, 1H), 7.03 (s, 2H), 6.11 (s, 2H), 4.85 (t, 1H), 4.16 (q, 2H), 3.56 (d, 2H), 1.35 (m, 9H). MS m/z calculated for (M+H)⁺ 403, found 403.

### 5.2.49 5-Amino-4-(3-methoxy-5-pyridyl)-2-(2-amino-2-methyl-1-propanol)-thieno[2,3-d]pyrimidine-6-carboxamide

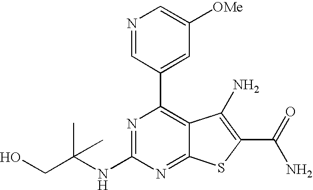

Step a: 3-Methoxy-5-formylpyridine. 3-Bromo-5-methoxypyridine (10.5 g, 55.9 mmol) was dissolved in tetrahydrofuran (70 mL) and cooled to 0° C. Isopropylmagnesiumchloride (31 mL, 2.0 M in THF) was syringed in and the reaction mixture was stirred for 2 hours at room temperature. N,N-dimethylformamide (8.5 mL) in tetrahydrofuran (12 mL) was added and stirring was continued for an additional hour. The solution was quenched with 2 N HCl to pH of 3 then partitioned between ethyl acetate and water. The organic layer was separated and concentrated to give a residue which was purified using flash chromatography to give 3-methoxy-5-formylpyridine (4.30 g, 56%) as an oil. ¹H NMR (DMSO-d₆): δ 10.1 (s, 1H), 8.71 (d, 1H), 8.59 (d, 1H), 7.74 (m, 1H), 3.92 (s, 3H).

Step b: 5-Cyano-4-(3-methoxy-5-pyridyl)-2-methylthio-6-oxopyrimidine. 3-Methoxy-5-formylpyridine (4.24 g, 30.9 mmol), ethylcyanoacetate (3.50 g, 30.9 mmol), S-methylisothiouronium sulfate (4.31 g, 15.4 mmol), and potassium carbonate (5.10 g, 36.9 mmol) was stirred in ethanol (200 mL) at 75° C. for 24 hours. The reaction mixture was cooled to 0° C. and the resulting solid was filtered, washed with ethanol, then transferred to water (500 mL). The solution was acidified to pH 3 with 2 N HCl and the solid produced was filtered and dried to give 5-cyano-4-(3-methoxy-5-pyridyl)-2-methylthio-6-oxopyrimidine (3.89 g, 45%) as a white solid. MS m/z calculated for (M+H)⁺ 275, found 275.

Step c: 6-Chloro-5-cyano-4-(3-methoxy-5-pyridyl)-2-methylthiopyrimidine. 5-Cyano-4-(3-methoxy-5-pyridyl)-2-methylthio-6-oxopyrimidine (3.80 g, 13.8 mmol) was stirred in dioxane (50 mL) and POCl₃ (50 mL). The mixture was heated to 90° C. for 3 hours then volatiles were removed and the residue was partitioned between ethyl acetate and sat. NaHCO₃. The organic layer was separated and concentrated to give 6-chloro-5-cyano-4-(3-methoxy-5-pyridyl)-2-methylthiopyrimidine (3.61 g, 90%) as a yellow solid. MS m/z calculated for (M+H)⁺ 293, found 293.

Step d: 5-Amino-4-(3-methoxy-5-pyridyl)-2-methylthio-thieno[2,3-d]pyrimidine-6-carboxamide. 6-chloro-5-cyano-4-(3-methoxy-5-pyridyl)-2-methylthiopyrimidine (3.60 g, 12.3 mmol), 2-mercaptoacetamide (1.20 g, 13.2 mmol), and sodium carbonate (1.57 g, 14.8 mmol) was stirred in ethanol (85 mL) at 75° C. for 16 hours. The reaction mixture was partitioned between ethyl acetate and water. The organic layer was separated and concentrated to give a residue. Excess sodium ethoxide in ethanol was added and the mixture was heated at 75° C. for 30 minutes. The reaction mixture was partitioned between ethyl acetate and water. The organic layer was separated and concentrated to give 5-Amino-4-(3-methoxy-5-pyridyl)-2-methylthio-thieno[2,3-d]pyrimidine-6-carboxamide (2.88 g, 67%) as a yellow solid. MS m/z calculated for (M+H)⁺ 348, found 348.

Step e: 5-Amino-4-(3-methoxy-5-pyridyl)-2-methylsulfoxide-thieno[2,3-d]pyrimidine-6-carboxamide. 5-Amino-4-(3-methoxy-5-pyridyl)-2-methylthio-thieno[2,3-d]pyrimidine-6-carboxamide (2.88 g, 8.30 mmol) was stirred in chloroform (475 mL) at −5° C. mCPBA (2.59 g, 14.9 mmol) was slowly added and stirring was continued for 60 minutes at −5° C. The reaction mixture was partitioned between chloroform and aqueous sodium bisulfite and sodium bicarbonate. The organic layer was separated and concentrated to give 5-Amino-4-(3-methoxy-5-pyridyl)-2-methylsulfoxide-thieno[2,3-d]pyrimidine-6-carboxamide (0.95 g, 32%) as a yellow solid. MS m/z calculated for (M+H)⁺ 364, found 364.

Step f: 5-Amino-4-(3-methoxy-5-pyridyl)-2-(2-amino-2-methyl-1-propanol)-thieno[2,3-d]pyrimidine-6-carboxamide. 5-Amino-4-(3-methoxy-5-pyridyl)-2-methylsulfoxide-thieno[2,3-d]pyrimidine-6-carboxamide (0.30 g, 0.82 mmol) and 2-amino-2-methyl-1-propanol (0.73 g, 8.26 mmol) was stirred in DMF (5 mL) at 160° C. for 45 minutes in a microwave. Volatiles were removed and the residue was purified using preparative HPLC to give the title compound 5-Amino-4-(3-methoxy-5-pyridyl)-2-(2-amino-2-methyl-1-propanol)-thieno[2,3-d]pyrimidine-6-carboxamide (0.035 g, 11%) as a yellow solid. ¹H NMR (DMSO-d₆): δ 8.42 (d, 1H), 8.34 (s, 1H), 7.56 (m, 1H), 7.09 (s, 1H), 7.00 (s, 2H), 6.10 (s, 2H), 4.82 (t, 1H), 3.85 (s, 3H), 3.53 (d, 2H), 1.35 (s, 6H). MS m/z calculated for (M+H)⁺ 389, found 389.

### 5.2.50 5-Amino-4-(3-methoxy-5-pyridyl)-2-(R-2-amino-1-butanol)-thieno[2,3-d]pyrimidine-6-carboxamide

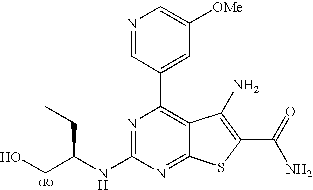

5-Amino-4-(3-methoxy-5-pyridyl)-2-methylsulfoxide-thieno[2,3-d]pyrimidine-6-carboxamide (0.20 g, 0.55 mmol) and (R)-(−)-2-amino-1-butanol (0.19 g, 2.20 mmol) was stirred in DMF (5 mL) at 75° C. for 60 minutes. The reaction mixture was partitioned between ethyl acetate and water. The organic layer was separated and concentrated to give a residue which was purified using flash chromatography (95% EtOAc, 5% ETOH) to give the title compound 5-amino-4-(3-methoxy-5-pyridyl)-2-(R-2-amino-1-butanol)-thieno[2,3-d]pyrimidine-6-carboxamide (0.13 g, 61%) as a yellow solid. ¹H NMR (DMSO-d₆): δ 8.34-8.44 (m, 2H), 7.53-7.59 (m, 2H), 7.01 (s, 2H), 6.05 (d, 2H), 4.67 (m, 1H), 3.87-4.00 (b, 4H), 3.39 (m, 2H), 1.45-1.69 (m, 2H), 0.89 (t, 3H). MS m/z calculated for (M+H)⁺ 389, found 389.

### 5.2.51 5-Amino-4-(3-methoxy-5-pyridyl)-2-(S-2-amino-1-butanol)-thieno[2,3-d]pyrimidine-6-carboxamide

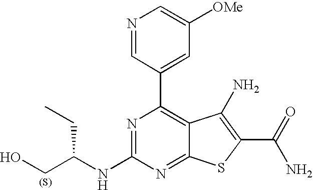

5-Amino-4-(3-methoxy-5-pyridyl)-2-methylsulfoxide-thieno[2,3-d]pyrimidine-6-carboxamide (0.10 g, 0.27 mmol) and (S)-(+)-2-amino-1-butanol (0.09 g, 1.10 mmol) was stirred in DMF (3 mL) at 75° C. for 60 minutes. The reaction mixture was partitioned between ethyl acetate and water. The organic layer was separated and concentrated to give a residue which was purified using flash chromatography (95% EtOAc, 5% ETOH) to give the title compound 5-amino-4-(3-methoxy-5-pyridyl)-2-(S-2-amino-1-butanol)-thieno[2,3-d]pyrimidine-6-carboxamide (0.03 g, 28%) as a yellow solid. ¹H NMR (DMSO-d₆): δ 8.34-8.44 (m, 2H), 7.53-7.60 (m, 2H), 7.01 (s, 2H), 6.05 (d, 2H), 4.67 (m, 1H), 3.87-4.00 (b, 4H), 3.39 (m, 2H), 1.45-1.69 (m, 2H), 0.89 (t, 3H). MS m/z calculated for (M+H)⁺ 389, found 389.

### 5.2.52 5-Amino-4-(3-methoxy-5-pyridyl)-2-(R-2-amino-1-propanol)-thieno[2,3-d]pyrimidine-6-carboxamide

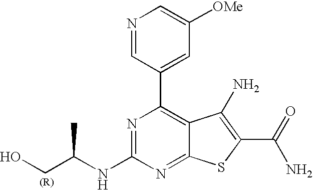

5-Amino-4-(3-methoxy-5-pyridyl)-2-methylsulfoxide-thieno[2,3-d]pyrimidine-6-carboxamide (0.23 g, 0.63 mmol) and (R)-(−)-2-amino-1-propanol (0.19 g, 2.51 mmol) was stirred in DMF (5 mL) at 75° C. for 60 minutes. The reaction mixture was partitioned between ethyl acetate and water. The organic layer was separated and concentrated to give a residue which was purified using flash chromatography (95% EtOAc, 5% ETOH) to give the title compound 5-amino-4-(3-methoxy-5-pyridyl)-2-(R-2-amino-1-propanol)-thieno[2,3-d]pyrimidine-6-carboxamide (0.11 g, 48%) as a yellow solid. ¹H NMR (DMSO-d₆): δ 8.34-8.45 (m, 2H), 7.43-7.59 (m, 2H), 7.01 (s, 2H), 6.06 (d, 2H), 4.70 (m, 1H), 4.05 (m, 1H), 3.87 (s, 3H), 3.39 (m, 2H), 1.15 (d, 3H). MS m/z calculated for (M+H)⁺ 375, found 375.

### 5.2.53 5-Amino-4-(3-methoxy-5-pyridyl)-2-(S-2-amino-1-propanol)-thieno[2,3-d]pyrimidine-6-carboxamide

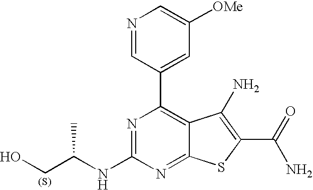

5-Amino-4-(3-methoxy-5-pyridyl)-2-methylsulfoxide-thieno[2,3-d]pyrimidine-6-carboxamide (0.11 g, 0.30 mmol) and (S)-(+)-2-amino-1-propanol (0.09 g, 1.21 mmol) was stirred in DMF (3 mL) at 75° C. for 60 minutes. The reaction mixture was partitioned between ethyl acetate and water. The organic layer was separated and concentrated to give a residue which was purified using flash chromatography (95% EtOAc, 5% ETOH) to give the title compound 5-amino-4-(3-methoxy-5-pyridyl)-2-(S-2-amino-1-propanol)-thieno[2,3-d]pyrimidine-6-carboxamide (0.014 g, 12%) as a yellow solid. ¹H NMR (DMSO-d₆): δ 8.34-8.45 (m, 2H), 7.46-7.59 (m, 2H), 7.01 (s, 2H), 6.06 (d, 2H), 4.70 (m, 1H), 4.05 (m, 1H), 3.87 (s, 3H), 3.39 (m, 2H), 1.15 (d, 3H). MS m/z calculated for (M+H)⁺ 375, found 375.

### 5.2.54 5-Amino-4-(3-ethyl-5-pyridyl)-2-(2-amino-2-methyl-1-propanol)-thieno[2,3-d]pyrimidine-6-carboxamide

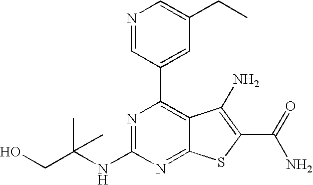

Step a: 3-Ethyl-5-formylpyridine. Commercially available 3-acetyl-5-bromopyridine (6.53 g, 32.8 mmol), solid NaOH (13.1 g, 326 mmol), and hydrazine hydrate (13 mL) was heated in diethylene glycol (25 mL) at 140° C. for 4 hours. The reaction mixture was partitioned between ether and water. The organic layer was separated and concentrated to give a residue which was purified using flash chromatography to give an oil. This was dissolved in tetrahydrofuran (40 μL) and cooled to 0° C. Isopropylmagnesiumchloride (20 mL, 2.0 M in THF) was syringed in and the reaction mixture was stirred for 2 hours at room temperature. N,N-dimethylformamide (7 mL) in tetrahydrofuran (15 mL) was added and stirring was continued for an additional hour. The solution was quenched with 2 N HCl to pH of 3 then partitioned between ethyl acetate and water. The organic layer was separated and concentrated to give a residue which was purified using flash chromatography to give 3-ethyl-5-formylpyridine (0.78 g, 18%) as a solid. ¹H NMR (DMSO-d₆): δ 10.1 (s, 1H), 8.91 (s, 1H), 8.71 (s, 1H), 8.00 (s, 1H), 2.75 (q, 2H), 1.31 (t, 3H). MS m/z calculated for (M+H)⁺ 152, found 152.

Step b: 5-Cyano-4-(3-ethyl-5-pyridyl)-2-methylthio-6-oxopyrimidine. 3-Ethyl-5-formylpyridine (0.78 g, 5.77 mmol), ethylcyanoacetate (0.65 g, 5.77 mmol), S-methylisothiouronium sulfate (0.80 g, 2.88 mmol), and potassium carbonate (0.95 g, 6.92 mmol) was stirred in ethanol (30 mL) at 75° C. for 24 hours. The reaction mixture was cooled to 0° C. and the resulting solid was filtered, washed with ethanol, then transferred to water (100 mL). The solution was acidified to pH 3 with 2 N HCl and the solid produced was filtered and dried to give 5-cyano-4-(3-ethyl-5-pyridyl)-2-methylthio-6-oxopyrimidine (0.85 g, 54%) as a white solid. MS m/z calculated for (M+H)⁺ 273, found 273.

Step c: 6-Chloro-5-cyano-4-(3-ethyl-5-pyridyl)-2-methylthiopyrimidine. 5-Cyano-4-(3-ethyl-5-pyridyl)-2-methylthio-6-oxopyrimidine (0.84 g, 3.10 mmol) was stirred in dioxane (15 mL) and POCl₃ (15 mL). The mixture was heated to 90° C. for 6 hours then volatiles were removed and the residue was partitioned between ethyl acetate and sat. NaHCO₃. The organic layer was separated and concentrated to give 6-chloro-5-cyano-4-(3-ethyl-5-pyridyl)-2-methylthiopyrimidine (0.54 g, 60%) as a yellow solid. MS m/z calculated for (M+H)⁺ 291, found 291.

Step d: 5-Amino-4-(3-ethyl-5-pyridyl)-2-methylthio-thieno[2,3-d]pyrimidine-6-carboxamide. 6-chloro-5-cyano-4-(3-ethyl-5-pyridyl)-2-methylthiopyrimidine (0.53 g, 1.83 mmol), 2-mercaptoacetamide (0.18 g, 2.01 mmol), was stirred in ethanol (15 mL) followed by the addition of at excess sodium ethoxide in ethanol. The mixture was heated at 75° C. for 1 hour. The reaction mixture was partitioned between ethyl acetate and water. The organic layer was separated and concentrated to give a residue which was purified using flash chromatography to give the title compound 5-Amino-4-(3-ethyl-5-pyridyl)-2-methylthio-thieno[2,3-d]pyrimidine-6-carboxamide (0.123 g, 20%) as a yellow solid. MS m/z calculated for (M+H)⁺ 346, found 346.

Step e: 5-Amino-4-(3-ethyl-5-pyridyl)-2-(2-amino-2-methyl-1-propanol)-thieno[2,3-d]pyrimidine-6-carboxamide. 5-Amino-4-(3-ethyl-5-pyridyl)-2-methylthio-thieno[2,3-d]pyrimidine-6-carboxamide (0.11 g, 0.31 mmol) was stirred in chloroform (20 mL) at 0° C. mCPBA (0.099 g, 0.57 mmol) was slowly added and stirring was continued for 60 minutes at 0° C. The reaction mixture was partitioned between chloroform and aqueous sodium bisulfite and sodium bicarbonate. The organic layer was separated and concentrated to give a residue which was taken up in DMF (5 mL). 2-amino-2-methyl-1-propanol (0.13 g, 1.40 mmol) was added and the reaction mixture heated at 65° C. for 24 hours. Volatiles were removed and the residue was purified using flash chromatography to give the title compound 5-Amino-4-(3-ethyl-5-pyridyl)-2-(2-amino-2-methyl-1-propanol)-thieno[2,3-d]pyrimidine-6-carboxamide (0.010 g, 8%) as a yellow solid. ¹H NMR (DMSO-d₆): δ 8.61 (d, 1H), 7.86 (s, 1H), 7.11 (s, 1H), 7.03 (s, 2H), 6.05 (s, 2H), 4.84 (m, 1H), 3.55 (s, 2H), 2.72 (q, 2H), 1.34 (s, 6H), 1.24 (t, 3H). MS m/z calculated for (M+H)⁺ 387, found 387.

### 5.2.55 5-Amino-4-(3-benzyloxy-5-pyridyl)-2-(2-amino-2-methyl-1-propanol)-thieno[2,3-d]pyrimidine-6-carboxamide

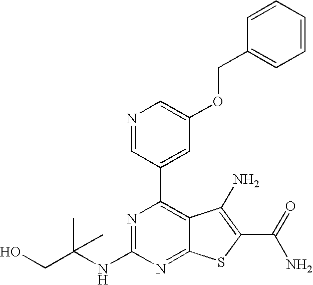

Step a: 3-Benzyloxy-5-bromopyridine. Benzyl alcohol (4.60 g, 42.6 mmol) was slowly added to a stirred solution of NaH (60% disp., 1.70 g, 42.5 mmol) in DMF (50 mL). The reaction was heated to 60° C. for 1 hours. 3,5-Dibromopyridine (10.0 g, 42.2 mmol) in DMF (20 mL) was added and the mixture was heated at 80° C. for 2 hours. The reaction mixture was partitioned between ethyl acetate and water. The organic layer was separated and concentrated to give a residue which was purified using flash chromatography to give 3-Benzyloxy-5-bromopyridine (4.75 g, 43%). MS m/z calculated for (M+H)⁺ 265, found 265.

Step b: 3-Benzyloxy-5-formylpyridine. 3-Benzyloxy-5-bromopyridine (4.73 g, 18.0 mmol) was dissolved in tetrahydrofuran (25 mL) and cooled to 0° C. Isopropylmagnesiumchloride (10 mL, 2.0 M in THF) was syringed in and the reaction mixture was stirred for 2 hrs at room temperature. N,N-dimethylformamide (3 mL) in tetrahydrofuran (10 mL) was added and stirring was continued for an additional hour. The solution was quenched with 2 N HCl to pH of 3 then partitioned between ethyl acetate and water. The organic layer was separated and concentrated to give a residue which was purified using flash chromatography to give 3-benzyloxy-5-formylpyridine (2.00 g, 53%) as white solid. ¹H NMR (DMSO-d₆): δ 10.1 (s, 1H), 8.72 (d, 1H), 8.66 (d, 1H), 7.84 (m, 1H), 7.42-7.48 (m, 5H), 5.28 (s, 2H).

Step c: 5-Cyano-4-(3-benzyloxy-5-pyridyl)-2-methylthio-6-oxopyrimidine. 3-Benzyloxy-5-formylpyridine (2.00 g, 9.39 mmol), ethylcyanoacetate (1.06 g, 9.39 mmol), S-methylisothiouronium sulfate (1.31 g, 4.69 mmol), and potassium carbonate (1.55 g, 11.3 mmol) was stirred in ethanol (60 mL) at 75° C. for 24 hours. The reaction mixture was cooled then transferred to water (300 mL). The solution was acidified to pH 3 with 2 N HCl and the solid produced was filtered and dried to give 5-cyano-4-(3-benzyloxy-5-pyridyl)-2-methylthio-6-oxopyrimidine (2.30 g, 70%) as a white solid. MS m/z calculated for (M+H)⁺ 351, found 351.

Step d: 6-Chloro-5-cyano-4-(3-benzyloxy-5-pyridyl)-2-methylthiopyrimidine. 5-Cyano-4-(3-benzyloxy-5-pyridyl)-2-methylthio-6-oxopyrimidine (2.30 g, 6.57 mmol) was stirred in dioxane (20 mL) and POCl₃ (20 mL). The mixture was heated to 80° C. for 2 hours then volatiles were removed and the residue was partitioned between ethyl acetate and sat. NaHCO₃. The organic layer was separated and concentrated to give 6-chloro-5-cyano-4-(3-benzyloxy-5-pyridyl)-2-methylthiopyrimidine (1.23 g, 51%). MS m/z calculated for (M+H)⁺ 369, found 369.

Step e: 5-Amino-4-(3-benzyloxy-5-pyridyl)-2-methylthio-thieno[2,3-d]pyrimidine-6-carboxamide. 6-chloro-5-cyano-4-(3-benzyloxy-5-pyridyl)-2-methylthiopyrimidine (1.20 g, 3.26 mmol), 2-mercaptoacetamide (0.31 g, 3.42 mmol), and sodium carbonate (0.36 g, 3.42 mmol) was stirred in ethanol (35 mL) at 60° C. for 18 hours. The reaction mixture was partitioned between ethyl acetate and water. The organic layer was separated and concentrated to give a residue. Excess sodium ethoxide in ethanol was added and the mixture was heated at 75° C. for 2 hours. The reaction mixture was cooled followed by filtration of the resulting solid to give 5-Amino-4-(3-benzyloxy-5-pyridyl)-2-methylthio-thieno[2,3-d]pyrimidine-6-carboxamide (0.94 g, 68%) as a yellow solid. MS m/z calculated for (M+H)⁺ 424, found 424.

Step f: 5-Amino-4-(3-benzyloxy-5-pyridyl)-2-methylsulfoxide-thieno[2,3-d]pyrimidine-6-carboxamide. 5-Amino-4-(3-benzyloxy-5-pyridyl)-2-methylthio-thieno[2,3-d]pyrimidine-6-carboxamide (0.93 g, 2.19 mmol) was stirred in chloroform (130 mL) at 0° C. mCPBA (0.71 g, 4.10 mmol) was slowly added and stirring was continued for 40 minutes at 0° C. The reaction mixture was partitioned between chloroform and aqueous sodium bisulfite and sodium bicarbonate. The organic layer was separated and concentrated to give 5-Amino-4-(3-benzyloxy-5-pyridyl)-2-methylsulfoxide-thieno[2,3-d]pyrimidine-6-carboxamide (0.62 g, 64%). MS m/z calculated for (M+H)⁺ 440, found 440.

Step g: 5-Amino-4-(3-benzyloxy-5-pyridyl)-2-(2-amino-2-methyl-1-propanol)-thieno[2,3-d]pyrimidine-6-carboxamide. 5-Amino-4-(3-benzyloxy-5-pyridyl)-2-methylsulfoxide-thieno[2,3-d]pyrimidine-6-carboxamide (0.61 g, 1.39 mmol) and 2-amino-2-methyl-1-propanol (1.00 g, 11.2 mmol) was stirred in DMF (15 mL) at 75° C. for 18 hours. The reaction mixture was partitioned between ethyl acetate and water. The organic layer was separated and concentrated to give a residue which was purified using flash chromatography to give the title compound 5-Amino-4-(3-benzyloxy-5-pyridyl)-2-(2-amino-2-methyl-1-propanol)-thieno[2,3-d]pyrimidine-6-carboxamide (0.19 g, 29%) as a yellow solid. ¹H NMR (DMSO-d₆): δ 8.52 (d, 1H), 8.38 (s, 1H), 7.69 (m, 1H), 7.35-7.49 (m, 5H), 7.11 (s, 1H), 7.03 (s, 2H), 6.07 (s, 2H), 5.23 (s, 2H), 4.84 (t, 1H), 3.55 (d, 2H), 1.33 (s, 6H). MS m/z calculated for (M+H)⁺ 465, found 465.

### 5.2.56 5-Amino-4-(3-hydroxy-5-pyridyl)-2-(2-amino-2-methyl-1-propanol)-thieno[2,3-d]pyrimidine-6-carboxamide

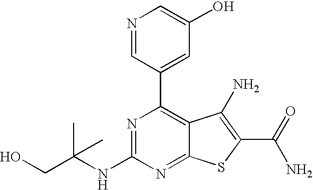

5-Amino-4-(3-benzyloxy-5-pyridyl)-2-(2-amino-2-methyl-1-propanol)-thieno[2,3-d]pyrimidine-6-carboxamide (0.15 g, 0.32 mmol) was stirred in MeOH (15 mL) and acetic acid (15 mL) with Pd/C under a balloon of hydrogen for 18 hours. The reaction mixture was filtered through a pad of Celite followed by prep HPLC purification to give 5-Amino-4-(3-hydroxy-5-pyridyl)-2-(2-amino-2-methyl-1-propanol)-thieno[2,3-d]pyrimidine-6-carboxamide (0.015 g, 13%) as a yellow solid. ¹H NMR (DMSO-d₆): δ 10.3 (s, 1H), 8.28 (d, 1H), 8.22 (s, 1H), 7.33 (s, 1H), 7.10 (s, 1H), 7.01 (s, 2H), 6.10 (s, 2H), 4.84 (t, 1H), 3.55 (d, 2H), 1.34 (s, 6H). MS m/z calculated for (M+H)⁺ 375, found 375.

### 5.2.57 5-Amino-2-(1-hydroxy-2-methylpropan-2-ylamino)-4-(4-methoxypyridin-2-yl)thieno[2,3-d]pyrimidine-6-carboxamide

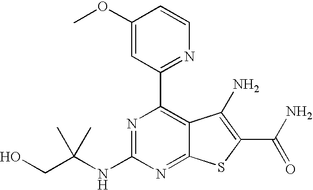

Step a: 4-Hydroxy-6-(4-methoxypyridin-2-yl)-2-(methylthio)pyrimidine-5-carbonitrile. 4-Methoxypicolinaldehyde (1.9 g, 13.85 mmol), methyl carbamimidothioate hemisulfate (1.93 g, 6.93 mmol), ethyl 2-cyanoacetate (1.57 g, 13.85 mmol) and potassium carbonate (2.30 g, 16.63 mmol) were mixed in 95 ml dry EtOH under N₂ and heated at 75° C. for two hours. The suspension was cooled to room temperature and solids were filtered. The solids were suspended in 50 ml H₂O and stirred for 30 minutes, filtered, and dried overnight to give the desired product as a red solid (1.04 g, 27%). ¹H NMR (400 MHz, DMSO-D6) δ ppm 2.37 (s, 3H), 3.88 (s, 3H), 7.05 (m, 1H), 7.43 (d, J=2.54 Hz, 1H), 8.45 (d, J=5.66 Hz, 1H). MS m/z calculated for (M+H)⁺ 275.31, found 275.

Step b: 4-Chloro-6-(4-methoxypyridin-2-yl)-2-(methylthio)pyrimidine-5-carbonitrile. 4-Hydroxy-6-(4-methoxypyridin-2-yl)-2-(methylthio)pyrimidine-5-carbonitrile (0.992 g, 3.62 mmol) was dissolved in 60 ml dry 1,4-dioxane under N₂ and phosphorus oxychloride (15.7 ml, 168.95 mmol) and a few drops of dry DMF were added. The reaction mixture was stirred overnight and the solvent evaporated. The reaction was quenched by adding ice/water and the aqueous layer was extracted with EtOAc. The organic phases were dried over MgSO₄ and evaporated to give the product as a red solid (0.976 g, 92%). ¹H NMR (400 MHz, DMSO-D6) δ ppm 2.50 (s, 3H), 3.95 (s, 3H), 7.31 (d, J=2.54 Hz, 1H), 7.82 (s, 1H), 8.63 (d, J=5.66 Hz, 1H). MS m/z calculated for (M+H)⁺ 293.75, found 293.

Step c: 5-Amino-4-(4-methoxypyridin-2-yl)-2-(methylthio)thieno[2,3-d]pyrimidine-6-carboxamide. 4-Chloro-6-(4-methoxypyridin-2-yl)-2-(methylthio)pyrimidine-5-carbonitrile (0.976 g, 3.33 mmol), 2-mercaptoacetamide (0.334, 3.67 mmol) and sodium carbonate (0.389 g, 3.67 mmol) were suspended in 30 ml dry EtOH under N₂ and stirred overnight. Solids were filtered and washed with a small amount of water. The solids were added to a solution of sodium (383 mg 16.65 mmol) in 10 ml dry EtOH under N₂ and heated at 70° C. for two hours. The reaction mixture was poured into water and extracted with EtOAc. The combined organic phases were dried over MgSO₄ and the solvent evaporated to give the product as a red solid (0.266 g, 23%). ¹H NMR (400 MHz, DMSO-D6) δ ppm 2.51 (s, 3H), 3.97 (s, 3H), 7.18 (m, 1H), 7.28 (m, 2H), 7.88 (m, 1H), 8.35 (m, 2H), 8.65 (m, 1H). MS m/z calculated for (M+H)⁺ 348.42, found 348.

Step d: 5-Amino-4-(4-methoxypyridin-2-yl)-2-(methylsulfinyl)thieno[2,3-d]pyrimidine-6-carboxamide. 5-Amino-4-(4-methoxypyridin-2-yl)-2-(methylthio)thieno[2,3-d]pyrimidine-6-carboxamide (236 mg, 0.679 mmol) was dissolved in dry CHCl₃ under N₂ and cooled to 0° C. 3-Chlorobenzoperoxoic acid (212 mg, 1.227 mmol) was added and the mixture was stirred at 0° C. for 75 minutes. The reaction was quenched with 10% aq. NaHSO₃. The phases were separated and the organic phase was washed with 10% aq. NaHCO₃. The organic phase was dried over MgSO₄ and the solvent evaporated to give the product as a red solid (0.198 g, 80%). MS m/z calculated for (M+H)⁺ 364.42, found 364. The compound was used directly for the next step.

Step e: 5-Amino-2-(1-hydroxy-2-methylpropan-2-ylamino)-4-(4-methoxypyridin-2-yl)thieno[2,3-d]pyrimidine-6-carboxamide. 5-Amino-4-(4-methoxypyridin-2-yl)-2-(methylsulfinyl)thieno[2,3-d]pyrimidine-6-carboxamide (198 mg, 0.545 mmol) and 2-amino-2-methylpropan-1-ol (486 mg, 5.45 mmol) were heated in dry DMF under N₂ at 90° C. for 18 hours. The solvent was evaporated and the residue was suspended in CH₂Cl₂. The solid was filtered and purified using HPLC (Method 10-100%, 30 minutes, 20 ml/min) to give the product as an orange solid (28 mg, 13%). ¹H NMR (400 MHz, DMSO-D6) δ ppm 1.23 (m, 2H), 1.37 (s, 3H), 3.58 (d, J=6.05 Hz, 2H), 3.95 (s, 3H), 4/86 (m, 1H), 6.92 (s, 2H), 7.07 (s, 1H), 7.24 (m, 1H), 7.82 (s, 1H), 8.59 (d, J=5.86 Hz, 1H). MS m/z calculated for (M+H)⁺ 389.45, found 389.

### 5.2.58 5-amino-2-(cyclopropylamino)-4-[2-(phenylmethoxy)phenyl]thiopheno[2,3-d]pyrimidine-6-carboxamide

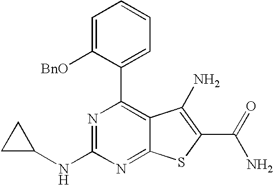

Step a: 2-(cyclopropylamino)-4-hydroxy-6-[2-(phenylmethoxy)phenyl]pyrimidine-5-carbonitrile. 2-(phenylmethoxy)benzaldehyde (2.0 g, 9.42 mmol), ethyl cyanoacetate (1.06 g, 9.42 mmol), cyclopropanecarboxamidine hydrochloride (1.27 g, 9.42 mmol), and potassium carbonate (1.3 g, 9.42 mmol) were refluxed in ethanol (60 mL) overnight. The stirbar was removed and water was added. The volatiles were removed and the aqueous layer was extracted with ethyl acetate, dried with sodium sulfate, and concentrated to give 2.78 g (82%) of the title compound as a yellow oil. MS m/z calculated for (M+H)⁺ 359, found 359.

Step b: 4-chloro-2-(cyclopropylamino)-6-[2-(phenylmethoxy)phenyl]pyrimidine-5-carbonitrile. 2-(cyclopropylamino)-4-hydroxy-6-[2-(phenylmethoxy)phenyl]pyrimidine-5-carbonitrile (2.78 g, 7.76 mmol) was dissolved in dioxane (50 mL) and POCl₃ (10 mL) was added. The resulting reaction mixture was heated at 90° C. under a reflux condenser for 2 h and then cooled to room temperature. The reaction mixture was added to ice and neutralized with saturated NaHCO₃. The aqueous layer was extracted with ethyl acetate, dried with sodium sulfate, and concentrated to give 2.67 g (92%) of the title compound as a yellow oil. MS m/z calculated for (M+H)⁺ 377, found 377.

Step c: 5-amino-2-(cyclopropylamino)-4-[2-(phenylmethoxy)phenyl]thiopheno[2,3-d]pyrimidine-6-carboxamide. 4-chloro-2-(cyclopropylamino)-6-[2-(phenylmethoxy)phenyl]pyrimidine-5-carbonitrile (2.67 g, 7.10 mmol), 2-mercaptoacetamide (646 mg, 7.10 mmol), Na₂CO₃ (753 mg, 7.10 mmol), and ethanol (35 mL) were combined in a flask, stirred vigorously, and heated at 70° C. under a reflux condenser for 18 hours. The reaction mixture was cooled to room temperature and water was added. The resulting precipitate was filtered and washed with Et₂O. The yellow solid was used directly in the next step without purification. 2-{5-cyano-2-(cyclopropylamino)-6-[2-(phenylmethoxy)phenyl]pyrimidin-4-ylthio}acetamide was dissolved in ethanol (15 mL). Freshly prepared NaOEt (35 mmol) in ethanol (15 mL) was added and the resulting mixture stirred at 70° C. for 1 hour. The reaction mixture was cooled to room temperature and quenched with saturated NaHCO₃. The aqueous layer was extracted with ethyl acetate, dried with sodium sulfate, and concentrated to give 1.5 g (49%) of the title compound as a yellow solid. ¹H NMR (300 MHz, DMSO-D6) δ ppm 0.43-0.56 (m, 2H) 0.65-0.76 (m, 2H) 2.78-2.88 (m, 1H) 5.15 (s, 2H) 5.87 (s, 2H) 6.95-7.04 (s, 2H) 7.05-7.31 (m, 3H) 7.24 (d, J=7.69 Hz, 1H) 7.36 (t, J=6.87 Hz, 1H) 7.94 (s, 2H). MS m/z calculated for (M+H)⁺ 432, found 432.

### 5.2.59 5-amino-2-(cyclopropylamino)-4-(2-hydroxyphenyl)thiopheno[2,3-d]pyrimidine-6-carboxamide

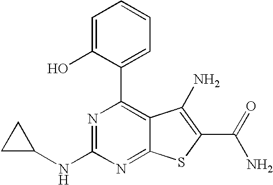

5-amino-2-(cyclopropylamino)-4-(2-hydroxyphenyl)thiopheno[2,3-d]pyrimidine-6-carboxamide. 5-amino-2-(cyclopropylamino)-4-[2-(phenylmethoxy)phenyl]thiopheno[2,3-d]pyrimidine-6-carboxamide (1.2 g, 2.77 mmol) was dissolved in MeOH (20 mL) and a catalytic amount of Pd(OH)₂ was added. The resulting reaction mixture was placed under 50 psi of H₂ for 16 hours. The reaction mixture was filtered through celite and concentrated. The residue was purified using reverse phase preparatory HPLC (30-100% CH₃CN/H₂O). CH₃CN was removed on a rotary evaporator. The resulting aqueous mixture was extracted with EtOAc and the organic layer was washed 5 times with saturated NaHCO₃ to remove TFA. The organic layer was dried with sodium sulfate and concentrated on a rotary evaporator. The resulting residue was triturated with EtOAc/hexane and dried under high vacuum to give 16 mg (2%) of the title compound as a yellow solid. ¹H NMR (300 MHz, DMSO-D6) δ ppm 0.43-0.56 (m, 2H) 0.65-0.76 (m, 2H) 2.73-2.85 (m, 1H) 5.87 (s, 2H) 6.85-7.04 (m, 3H) 7.24 (d, J=7.69 Hz, 1H) 7.36 (t, J=6.87 Hz, 1H) 7.90 (s, 2H). MS m/z calculated for (M+H)⁺ 342, found 342.

### 5.2.60 2,5-diamino-4-(3-methoxyphenyl)thiopheno[2,3-d]pyrimidine-6-carboxamide

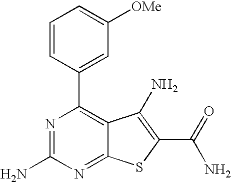

Step a: 2-amino-4-hydroxy-6-(3-methoxyphenyl)pyrimidine-5-carbonitrile. 3-methoxybenzaldehyde (5.0 g, 36.72 mmol), ethyl cyanoacetate (4.15 g, 36.72 mmol), guanidine hydrochloride (3.51 g, 36.72 mmol), and potassium carbonate (5.07 g, 36.72 mmol) were refluxed in ethanol (100 mL) overnight. The stirbar was removed and water was added. The volatiles were removed and the aqueous layer was acidified to pH 4-5 with 1N HCl. The precipitate was filtered and washed with water. The filter cake was dried under vacuum to give 1.58 g (18%) of the title compound as a white solid. MS m/lz calculated for (M+H)⁺ 243, found 243.

Step b: 2-amino-4-chloro-6-(3-methoxyphenyl)pyrimidine-5-carbonitrile. To 2-amino-4-hydroxy-6-(3-methoxyphenyl)pyrimidine-5-carbonitrile (1.48 g, 6.12 mmol) was added POCl₃ (10 mL). The resulting reaction mixture was heated at 90° C. under a reflux condenser for 1 h and then cooled to room temperature. The reaction mixture was added to ice and neutralized with saturated NaHCO₃. The aqueous layer was extracted with ethyl acetate, dried with sodium sulfate, and concentrated to give 1.84 g (100%) of the title compound as a yellow oil. MS m/z calculated for (M+H)⁺ 261, found 261.

Step c: 2,5-diamino-4-(3-methoxyphenyl)thiopheno[2,3-d]pyrimidine-6-carboxamide. 2-amino-4-chloro-6-(3-methoxyphenyl)pyrimidine-5-carbonitrile (1.59 g, 6.09 mmol), 2-mercaptoacetamide (554 mg, 6.09 mmol), Na₂CO₃ (646 mg, 6.09 mmol), and ethanol (35 mL) were combined in a flask, stirred vigorously, and heated at 70° C. under a reflux condenser for 6 hours. The reaction mixture was cooled to room temperature and water was added. EtOH was removed on a rotary evaporator and the aqueous layer was extracted with ethyl acetate, dried with sodium sulfate, and concentrated. The resulting oil was used in the next step without further purification. 2-[2-amino-5-cyano-6-(3-methoxyphenyl)pyrimidin-4-ylthio]acetamide was dissolved in ethanol (15 mL). Freshly prepared NaOEt (30 mmol) in ethanol (10 mL) was added and the resulting mixture stirred at 70° C. for 2 hours. The reaction mixture was cooled to room temperature and quenched with saturated NaHCO₃. The aqueous layer was extracted with ethyl acetate, dried with sodium sulfate, and concentrated. The residue was purified using reverse phase preparatory HPLC (30-100% CH₃CN/H₂O). CH₃CN was removed on a rotary evaporator. The resulting aqueous mixture was extracted with EtOAc and the organic layer was washed 5 times with saturated NaHCO₃ to remove TFA. The organic layer was dried with sodium sulfate and concentrated on a rotary evaporator. The resulting residue was triturated with EtOAc/hexane and dried under high vacuum to give 30 mg (2%) of the title compound as a yellow solid. ¹H NMR (300 MHz, DMSO-D6) δ ppm 3.81 (s, 3H) 6.01 (s, 2H) 6.97 (s, 2H) 7.05-7.16 (m, 4H) 7.19 (s, 1H) 7.47 (t, J=8.10 Hz, 1H). MS m/z calculated for (M+H)⁺ 316, found 316.

### 5.2.61 2,5-diamino-4-(3-hydroxyphenyl)thiopheno[2,3-d]pyrimidine-6-carboxamide

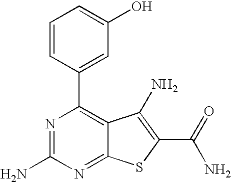

Step a: 2-amino-4-hydroxy-6-[3-(phenylmethoxy)phenyl]pyrimidine-5-carbonitrile. 3-(phenylmethoxy)benzaldehyde (5.0 g, 23.56 mmol), ethyl cyanoacetate (2.67 g, 23.56 mmol), guanidine hydrochloride (2.25 g, 23.56 mmol), and potassium carbonate (3.26 g, 23.56 mmol) were refluxed in ethanol (100 mL) overnight. The stirbar was removed and the white solid was filtered and washed with ethanol. The filter cake was dried under vacuum to give 5.56 g (74%) of the title compound as a white solid. ¹H NMR (400 MHz, DMSO-D6) δ ppm 5.12 (s, 2H) 5.74 (s, 2H) 6.99-7.10 (m, 1H) 7.27-7.37 (m, 4H) 7.37-7.43 (m, 2H) 7.43-7.52 (m, 2H). MS m/z calculated for (M+H)⁺ 319, found 319.

Step b: 2-amino-4-chloro-6-[3-(phenylmethoxy)phenyl]pyrimidine-5-carbonitrile. To 2-amino-4-hydroxy-6-[3-(phenylmethoxy)phenyl]pyrimidine-5-carbonitrile (5.54 g, 17.42 mmol) was added POCl₃ (10 mL). The resulting reaction mixture was heated at 90° C. under a reflux condenser for 2 h and then cooled to room temperature. The reaction mixture was added to ice and neutralized with saturated NaHCO₃. The aqueous layer was extracted with ethyl acetate, dried with sodium sulfate, and concentrated to give 1.2 g (20%) of the title compound and debenzylated material as a mixture. MS m/z calculated for (M+H)⁺ 337, found 337.

Step c: 2,5-diamino-4-(3-hydroxyphenyl)thiopheno[2,3-d]pyrimidine-6-carboxamide. 2-amino-4-chloro-6-[3-(phenylmethoxy)phenyl]pyrimidine-5-carbonitrile (1.2 g, 3.56 mmol), 2-mercaptoacetamide (324 mg, 3.56 mmol), Na₂CO₃ (377 mg, 3.56 mmol), and ethanol (35 mL) were combined in a flask, stirred vigorously, and heated at 70° C. under a reflux condenser for 16 hours. The reaction mixture was cooled to room temperature and water was added. EtOH was removed on a rotary evaporator and the aqueous layer was extracted with ethyl acetate, dried with sodium sulfate, and concentrated. The resulting oil was used in the next step without further purification. 2-{2-amino-5-cyano-6-[3-(phenylmethoxy)phenyl]pyrimidin-4-ylthio}acetamide was dissolved in ethanol (15 mL). Freshly prepared NaOEt (17.8 mmol) in ethanol (10 mL) was added and the resulting mixture stirred at 70° C. for 2 hours. The reaction mixture was cooled to room temperature and quenched with saturated NaHCO₃. The aqueous layer was extracted with ethyl acetate, dried with sodium sulfate, and concentrated. The residue was purified using reverse phase preparatory HPLC (20-100% CH₃CN/H₂O). CH₃CN was removed on a rotary evaporator. The resulting aqueous mixture was extracted with EtOAc and the organic layer was washed 5 times with saturated NaHCO₃ to remove TFA. The organic layer was dried with sodium sulfate and concentrated on a rotary evaporator. The resulting residue was triturated with EtOAc/hexane and dried under high vacuum to give 10 mg (1%) of the title compound as a yellow solid. 1H NMR (300 MHz, DMSO-D6) δ ppm 6.03 (s, 2H) 6.74-7.05 (m, 3H) 7.17 (s, 2H) 7.23-7.46 (m, 1H) 9.82 (s, 1H). MS m/z calculated for (M+H)⁺ 302, found 302.

### 5.2.62 5-amino-2-(cyclopropylamino)-4-(2-fluorophenyl)thiopheno[2,3-d]pyrimidine-6-carboxamide

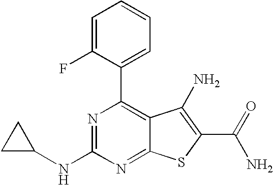

Step a: 2-(cyclopropylamino)-6-(2-fluorophenyl)-4-hydroxypyrimidine-5-carbonitrile. 2-fluorobenzaldehyde (2.5 g, 20.14 mmol), ethyl cyanoacetate (2.28 g, 20.14 mmol), cyclopropanecarboxamidine hydrochloride (2.72 g, 20.14 mmol), and potassium carbonate (2.78 g, 20.14 mmol) were refluxed in ethanol (60 mL) overnight. The stirbar was removed and water was added. The volatiles were removed and the aqueous layer was extracted with ethyl acetate, dried with sodium sulfate, and concentrated to give 4.96 g (91%) of the title compound as a yellow oil. MS m/z calculated for (M+H)⁺ 271, found 271.

Step b: 4-chloro-2-(cyclopropylamino)-6-(2-fluorophenyl)pyrimidine-5-carbonitrile. 2-(cyclopropylamino)-6-(2-fluorophenyl)-4-hydroxypyrimidine-5-carbonitrile (3.76 g, 13.93 mmol) was dissolved in dioxane (50 mL) and POCl₃ (10 mL) was added. The resulting reaction mixture was heated at 90° C. under a reflux condenser for 2 h and then cooled to room temperature. The reaction mixture was added to ice and neutralized with saturated NaHCO₃. The aqueous layer was extracted with ethyl acetate, dried with sodium sulfate, and concentrated to give 4.02 g (100%) of the title compound as a yellow oil. MS m/z calculated for (M+H)⁺ 289, found 289.

Step c: 5-amino-2-(cyclopropylamino)-4-(2-fluorophenyl)thiopheno[2,3-d]pyrimidine-6-carboxamide. 4-chloro-2-(cyclopropylamino)-6-(2-fluorophenyl)pyrimidine-5-carbonitrile (4.01 g, 13.93 mmol), 2-mercaptoacetamide (1.27 mg, 13.93 mmol), Na₂CO₃ (1.48 mg, 13.93 mmol), and ethanol (40 mL) were combined in a flask, stirred vigorously, and heated at 70° C. under a reflux condenser for 16 hours. The reaction mixture was cooled to room temperature and water was added. EtOH was removed on a rotary evaporator and the aqueous layer was extracted with ethyl acetate, dried with sodium sulfate, and concentrated. The resulting oil was used in the next step without further purification. 2-[5-cyano-2-(cyclopropylamino)-6-(2-fluorophenyl)pyrimidin-4-ylthio]acetamide was dissolved in ethanol (15 mL). Freshly prepared NaOEt (17.8 mmol) in ethanol (10 mL) was added and the resulting mixture stirred at 70° C. for 2 hours. The reaction mixture was cooled to room temperature and quenched with saturated NaHCO₃. The aqueous layer was extracted with ethyl acetate, dried with sodium sulfate, and concentrated. The residue was purified using reverse phase preparatory HPLC (30-100% CH₃CN/H₂O). CH₃CN was removed on a rotary evaporator. The resulting aqueous mixture was extracted with EtOAc and the organic layer was washed 5 times with saturated NaHCO₃ to remove TFA. The organic layer was dried with sodium sulfate and concentrated on a rotary evaporator. The resulting residue was triturated with EtOAc/hexane and dried under high vacuum to give 20 mg (1%) of the title compound as a yellow solid. ¹H NMR (400 MHz, DMSO-D6) δ ppm 0.47-0.55 (m, 2H) 0.64-0.76 (m, 2H) 2.73-2.85 (m, 1H) 5.80 (s, 2H) 7.03 (s, 2H) 7.31-7.46 (m, 2H) 7.54 (t, J=8.20 Hz, 1H) 7.57-7.67 (m, 1H). MS m/z calculated for (M+H)⁺ 344, found 344.

### 5.2.63 5-amino-4-(2,6-difluorophenyl)-2-(cyclopropylamino)thiopheno[2,3-d]pyrimidine-6-carboxamide

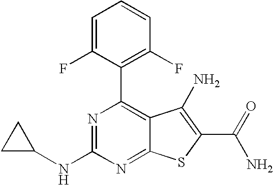

Step a: 6-(2,6-difluorophenyl)-2-(cyclopropylamino)-4-hydroxypyrimidine-5-carbonitrile. 2,6-difluorobenzaldehyde (1.5 g, 10.48 mmol), ethyl cyanoacetate (1.18 g, 10.48 mmol), cyclopropanecarboxamidine hydrochloride (1.41 g, 10.48 mmol), and potassium carbonate (1.45 g, 10.48 mmol) were refluxed in ethanol (40 mL) overnight. The stirbar was removed and water was added. The volatiles were removed and the aqueous layer was extracted with ethyl acetate, dried with sodium sulfate, and concentrated to give 2.0 g (66%) of the title compound as a yellow oil. MS m/z calculated for (M+H)⁺ 289, found 289.

Step b: 6-(2,6-difluorophenyl)-4-chloro-2-(cyclopropylamino)pyrimidine-5-carbonitrile. 6-(2,6-difluorophenyl)-2-(cyclopropylamino)-4-hydroxypyrimidine-5-carbonitrile (2.0 g, 6.94 mmol) was dissolved in dioxane (30 mL) and POCl₃ (10 mL) was added. The resulting reaction mixture was heated at 90° C. under a reflux condenser for 2 h and then cooled to room temperature. The reaction mixture was added to ice and neutralized with saturated NaHCO₃. The aqueous layer was extracted with ethyl acetate, dried with sodium sulfate, and concentrated to give 2.48 g (100%) of the title compound as a yellow oil. MS m/z calculated for (M+H)⁺ 307, found 307.

Step c: 5-amino-4-(2,6-difluorophenyl)-2-(cyclopropylamino)thiopheno[2,3-d]pyrimidine-6-carboxamide. 6-(2,6-difluorophenyl)-4-chloro-2-(cyclopropylamino)pyrimidine-5-carbonitrile (2.0 g, 6.54 mmol), 2-mercaptoacetamide (594 mg, 6.54 mmol), Na₂CO₃ (693 mg, 6.54 mmol), and ethanol (40 mL) were combined in a flask, stirred vigorously, and heated at 70° C. under a reflux condenser for 2 hours. The reaction mixture was cooled to room temperature and water was added. EtOH was removed on a rotary evaporator and the aqueous layer was extracted with ethyl acetate, dried with sodium sulfate, and concentrated. The resulting oil was used in the next step without further purification. 2-[5-cyano-2-(cyclopropylamino)-6-(2,6-difluorophenyl)pyrimidin-4-ylthio]acetamide was dissolved in ethanol (15 mL). Freshly prepared NaOEt (17.8 mmol) in ethanol (10 mL) was added and the resulting mixture stirred at 70° C. for 2 hours. The reaction mixture was cooled to room temperature and quenched with saturated NaHCO₃. The aqueous layer was extracted with ethyl acetate, dried with sodium sulfate, and concentrated. The residue was purified using reverse phase preparatory HPLC (25-75% CH₃CN/H₂O). CH₃CN was removed on a rotary evaporator. The resulting aqueous mixture was extracted with EtOAc and the organic layer was washed 5 times with saturated NaHCO₃ to remove TFA. The organic layer was dried with sodium sulfate and concentrated on a rotary evaporator. The resulting residue was triturated with CH₃CN/H₂O and the yellow powder filtered and dried under high vacuum to give 36 mg (2%) of the title compound as a yellow solid. ¹H NMR (400 MHz, DMSO-D6) δ ppm 0.47-0.57 (m, 2H) 0.66-0.76 (m, 2H) 2.72-2.87 (m, 1H) 5.90 (s, 2H) 7.08 (s, 2H) 7.25-7.36 (m, 1H) 7.60-7.73 (m, 1H) 8.14 (s, 1H). MS m/z calculated for (M+H)⁺ 362, found 362.

### 5.2.64 5-amino-4-(3,4-dimethylphenyl)-2-(cyclopropylamino)thiopheno[2,3-d]pyrimidine-6-carboxamide

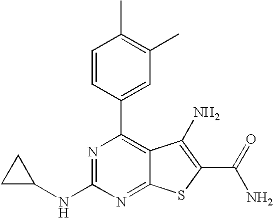

Step a: 6-(3,4-dimethylphenyl)-2-(cyclopropylamino)-4-hydroxypyrimidine-5-carbonitrile. 3,4-dimethylbenzaldehyde (1.5 g, 11.18 mmol), ethyl cyanoacetate (1.26 g, 11.18 mmol), cyclopropanecarboxamidine hydrochloride (1.51 g, 11.18 mmol), and potassium carbonate (1.54 g, 11.18 mmol) were refluxed in ethanol (40 mL) overnight. The stirbar was removed and water was added. The volatiles were removed and the aqueous layer was extracted with ethyl acetate, dried with sodium sulfate, and concentrated to give 1.8 g (58%) of the title compound as a yellow oil. MS m/z calculated for (M+H)⁺ 281, found 281.

Step b: 6-(3,4-dimethylphenyl)-4-chloro-2-(cyclopropylamino)pyrimidine-5-carbonitrile. 6-(3,4-dimethylphenyl)-2-(cyclopropylamino)-4-hydroxypyrimidine-5-carbonitrile (1.8 g, 6.42 mmol) was dissolved in dioxane (30 mL) and POCl₃ (10 mL) was added. The resulting reaction mixture was heated at 90° C. under a reflux condenser for 2 h and then cooled to room temperature. The reaction mixture was added to ice and neutralized with saturated NaHCO₃. The aqueous layer was extracted with ethyl acetate, dried with sodium sulfate, and concentrated to give 1.65 g (86%) of the title compound as a yellow oil. MS m/z calculated for (M+H)⁺ 299, found 299.

Step c: 5-amino-4-(3,4-dimethylphenyl)-2-(cyclopropylamino)thiopheno[2,3-d]pyrimidine-6-carboxamide. 6-(3,4-dimethylphenyl)-4-chloro-2-(cyclopropylamino)pyrimidine-5-carbonitrile (1.65 g, 5.54 mmol), 2-mercaptoacetamide (504 mg, 5.54 mmol), Na₂CO₃ (587 mg, 5.54 mmol), and ethanol (30 mL) were combined in a flask, stirred vigorously, and heated at 70° C. under a reflux condenser for 2 hours. The reaction mixture was cooled to room temperature and water was added. EtOH was removed on a rotary evaporator and the aqueous layer was extracted with ethyl acetate, dried with sodium sulfate, and concentrated. The resulting oil was used in the next step without further purification. 2-[5-cyano-2-(cyclopropylamino)-6-(3,4-dimethylphenyl)pyrimidin-4-ylthio]acetamide was dissolved in ethanol (15 mL). Freshly prepared NaOEt (17.8 mmol) in ethanol (10 mL) was added and the resulting mixture stirred at 70° C. for 2 hours. The reaction mixture was cooled to room temperature and quenched with saturated NaHCO₃. The aqueous layer was extracted with ethyl acetate, dried with sodium sulfate, and concentrated. The resulting residue was triturated with CH₃CN and filtered. The solid was then suspended in EtOAc/EtOH and washed with EtOH to give 75 mg (4%) of the title compound as a yellow solid. ¹H NMR (300 MHz, DMSO-D6) δ ppm 0.43-0.57 (m, 2H) 0.62-0.78 (m, 2H) 2.31 (d, J=4.40 Hz, 6H) 2.71-2.90 (m, 1H) 6.02 (s, 2H) 6.98 (s, 2H) 7.20-7.43 (m, 3H) 7.90 (s, 1H) MS m/z calculated for (M+H)⁺ 354, found 354.

### 5.2.65 5-amino-4-(3,4-dichlorophenyl)-2-cyclopropylthiopheno[2,3-d]pyrimidine-6-carboxamide

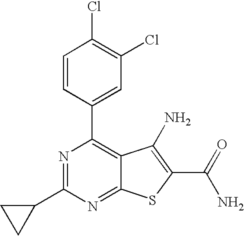

Step a: 6-(3,4-dichlorophenyl)-2-cyclopropyl-4-hydroxypyrimidine-5-carbonitrile. 3,4-dichlorobenzaldehyde (2.0 g, 11.43 mmol), ethyl cyanoacetate (1.29 g, 11.43 mmol), cyclopropanecarboxamidine hydrochloride (1.38 g, 11.43 mmol), and potassium carbonate (1.58 g, 11.43 mmol) were refluxed in ethanol (40 mL) overnight. The stirbar was removed and water was added. The precipitate was filtered and washed with water to give 2.17 g (62%) of the title compound as a white powder. MS m/z calculated for (M+H)⁺ 306, found 306.

Step b: 6-(3,4-dichlorophenyl)-4-chloro-2-cyclopropylpyrimidine-5-carbonitrile. 6-(3,4-dichlorophenyl)-2-cyclopropyl-4-hydroxypyrimidine-5-carbonitrile (2.10 g, 6.88 mmol) was dissolved in dioxane (30 mL) and POCl₃ (10 mL) was added. The resulting reaction mixture was heated at 90° C. under a reflux condenser for 2 h and then cooled to room temperature. The reaction mixture was added to ice and neutralized with saturated NaHCO₃. The aqueous layer was extracted with ethyl acetate, dried with sodium sulfate, and concentrated to give 1.0 g (45%) of the title compound as a white solid. MS m/z calculated for (M+H)⁺ 324, found 324.

Step c: 5-amino-4-(3,4-dichlorophenyl)-2-cyclopropylthiopheno[2,3-d]pyrimidine-6-carboxamide. 6-(3,4-dichlorophenyl)-4-chloro-2-cyclopropylpyrimidine-5-carbonitrile (1.0 g, 3.10 mmol), 2-mercaptoacetamide (282 mg, 3.10 mmol), Na₂CO₃ (329 mg, 3.10 mmol), and ethanol (30 mL) were combined in a flask, stirred vigorously, and heated at 70° C. under a reflux condenser for 16 hours. The reaction mixture was cooled to room temperature and water was added. The resulting precipitate was filtered and washed with water to give a white solid that was used without purification in the next step. 2-[6-(3,4-dichlorophenyl)-5-cyano-2-cyclopropylpyrimidin-4-ylthio]acetamide was dissolved in ethanol (15 mL). Freshly prepared NaOEt (17.8 mmol) in ethanol (10 mL) was added and the resulting mixture stirred at 70° C. for 2 hours. The reaction mixture was cooled to room temperature and quenched with saturated NaHCO₃. The resulting yellow precipitate was filtered and recrystallized from EtOAc/Hexane to give 600 mg (51%) of the title compound as a yellow crystalline solid. ¹H NMR (300 MHz, DMSO-D6) δ ppm 1.09-1.15 (m, 4H) 2.27-2.36 (m, 1H) 6.28 (s, 2H) 7.34 (s, 2H) 7.64 (dd, J=8.24, 1.92 Hz, 1H) 7.82 (d, J=8.24 Hz, 1H) 7.92 (d, J=1.92 Hz, 1H). MS m/z calculated for (M+H)⁺ 379, found 379.

### 5.2.66 5-amino-4-(5-chloro-3-hydroxyphenyl)-2-[(2-hydroxy-tert-butyl)amino]thiopheno[2,3-d]pyrimidine-6-carboxamide

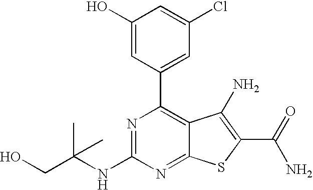

Step a: 6-(3-chloro-5-methoxyphenyl)-4-hydroxy-2-methylthiopyrimidine-5-carbonitrile. 3-chloro-5-methoxybenzaldehyde (6.52 g, 38.13 mmol), ethyl cyanoacetate (4.31 g, 38.13 mmol), methylthiocarboxamidine (10.61 g, 38.13 mmol), and potassium carbonate (5.26 g, 38.13 mmol) were refluxed in ethanol (100 mL) overnight. The stirbar was removed and water was added. The volatiles were removed and the aqueous layer was extracted with ethyl acetate, dried with sodium sulfate, and concentrated to give 12 g (100%) of the title compound as a brown oil. MS m/z calculated for (M+H)⁺ 308, found 308.

Step b: 4-chloro-6-(3-chloro-5-methoxyphenyl)-2-methylthiopyrimidine-5-carbonitrile. 6-(3-chloro-5-methoxyphenyl)-4-hydroxy-2-methylthiopyrimidine-5-carbonitrile (11.71 g, 38.14 mmol) was dissolved in dioxane (200 mL) and POCl₃ (40 mL) was added. After the addition of DMF (3 mL), the resulting reaction mixture was heated at 90° C. under a reflux condenser for 4 h and then cooled to room temperature. The volatiles were removed and the remaining reaction mixture was added to ice and neutralized with saturated NaHCO₃. The aqueous layer was extracted with ethyl acetate, dried with sodium sulfate, and concentrated. The residue was purified using flash chromatography (1:1 EtOAc/hexane) to give 3.5 g (28%) of the title compound as an off-white solid. 1H NMR (300 MHz, DMSO-D6) δ ppm 2.65 (s, 3H) 3.87 (s, 3H) 7.37 (t, J=2.20 Hz, 1H) 7.44-7.50 (m, 1H) 7.55 (t, J=1.65 Hz, 1H). MS m/z calculated for (M+H)⁺ 326, found 326.

Step c: 5-amino-4-(3-chloro-5-methoxyphenyl)-2-methylthiothiopheno[2,3-d]pyrimidine-6-carboxamide. 4-chloro-6-(3-chloro-5-methoxyphenyl)-2-methylthiopyrimidine-5-carbonitrile (3.5 g, 10.77 mmol), 2-mercaptoacetamide (980 mg, 10.77 mmol), Na₂CO₃ (1140 mg, 10.77 mmol), and ethanol (60 mL) were combined in a flask, stirred vigorously, and heated at 70° C. under a reflux condenser for 5 hours. The reaction mixture was cooled to room temperature and water was added. The resulting precipitate was filtered and washed with water to give a white solid that was used without purification in the next step. 2-[6-(3-chloro-5-methoxyphenyl)-5-cyano-2-methylthiopyrimidin-4-ylthio]acetamide was dissolved in ethanol (15 mL). Freshly prepared NaOEt (17.8 mmol) in ethanol (10 mL) was added and the resulting mixture stirred at 70° C. for 1 hour. The reaction mixture was cooled to room temperature and quenched with saturated NaHCO₃. The resulting yellow precipitate was filtered to give 3.48 g (85%) of the title compound as a yellow solid. 1H NMR (300 MHz, DMSO-D6) δ ppm 2.60 (s, 3H) 3.84 (s, 3H) 6.21 (s, 2H) 7.12-7.22 (m, 1H) 7.23-7.31 (m, 2H) 7.34 (s, 2H). MS m/z calculated for (M+H)⁺ 381, found 381.

Step d: 5-amino-4-(3-chloro-5-methoxyphenyl)-2-(methylsulfinyl)thiopheno[2,3-d]pyrimidine-6-carboxamide. 5-amino-4-(3-chloro-5-methoxyphenyl)-2-methylthiothiopheno[2,3-d]pyrimidine-6-carboxamide (3.48 g, 9.16 mmol) was dissolved in CHCl₃ (15 mL) and the reaction was cooled to 0° C. mCPBA (5.0 g, 22.89 mmol) was added and the reaction stirred at 0° C. for 1 hour. NaHSO₃ (10% solution) was added and the reaction stirred at room temperature for 1 hour. The organic layer was extracted and washed with saturated NaHCO₃, dried with sodium sulfate, and concentrated to give 2.46 g (68%) of the title compound as a yellow powder. MS m/z calculated for (M+H)⁺ 397, found 397.

Step e: 5-amino-4-(3-chloro-5-methoxyphenyl)-2-[(2-hydroxy-tert-butyl)amino]thiopheno[2,3-d]pyrimidine-6-carboxamide. 5-amino-4-(3-chloro-5-methoxyphenyl)-2-(methylsulfinyl)thiopheno[2,3-d]pyrimidine-6-carboxamide (500 mg, 1.26 mmol) was dissolved in DMF (4 mL) and 2-amino-2-methylpropan-1-ol (450 mg, 5.05 mmol) was added. The reaction proceeded under microwave irradiation at 170° C. for 15 minutes. The residue was purified using reverse phase preparatory HPLC (20-100% CH₃CN/H₂O). CH₃CN was removed on a rotary evaporator. The resulting aqueous mixture was extracted with EtOAc and the organic layer was washed 5 times with saturated NaHCO₃ to remove TFA. The organic layer was dried with sodium sulfate and concentrated on a rotary evaporator to give 120 mg (23%) of the title compound as a yellow solid. MS m/z calculated for (M+H)⁺ 422, found 422.

Step f: 5-amino-4-(5-chloro-3-hydroxyphenyl)-2-[(2-hydroxy-tert-butyl)amino]thiopheno[2,3-d]pyrimidine-6-carboxamide. 5-amino-4-(3-chloro-5-methoxyphenyl)-2-[(2-hydroxy-tert-butyl)amino]thiopheno[2,3-d]pyrimidine-6-carboxamide (120 mg, 285 mmol) was dissolved in CH₂Cl₂ (10 mL) and BBr₃ (1.43 mL) was added. The reaction was stirred at room temperature for 18 h when an additional 2 mL of BBr₃ was added. After an additional 5 h, the mixture was added to ice and neutralized with saturated NaHCO₃. The aqueous layer was extracted with ethyl acetate, dried with sodium sulfate, and concentrated. The residue was purified using reverse phase preparatory HPLC (10-65% CH₃CN/H₂O), then re-purified using reverse phase semipreperative HPLC (10-70% CH₃CN/H₂O). CH₃CN was removed on a rotary evaporator. The resulting aqueous mixture was extracted with EtOAc and the organic layer was washed 5 times with saturated NaHCO₃ to remove TFA. The organic layer was dried with sodium sulfate and concentrated on a rotary evaporator. The resulting residue was triturated with EtOAc/Hexane and the yellow powder filtered and dried under high vacuum to give 10 mg (8%) of the title compound as a yellow solid. 1H NMR (400 MHz, DMSO-D6) δ ppm 1.33 (s, 6H) 3.54 (d, J=6.25 Hz, 1H) 6.10 (s, 2H) 6.89 (s, 2H) 6.97 (t, J=2.15 Hz, 1H) 6.99 (s, 1H) 7.01-7.06 (m, 1H) 7.09 (s, 1H). MS m/z calculated for (M+H)⁺ 408, found 408.

### 5.2.67 2-[((1S)-1-ethyl-2-hydroxyethyl)amino]-5-amino-4-(5-chloro-3-hydroxyphenyl)thiopheno[2,3-d]pyrimidine-6-carboxamide

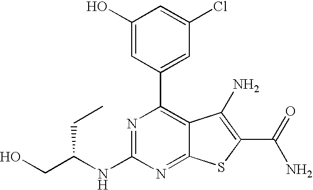

Step a: 2-[((1S)-1-ethyl-2-hydroxyethyl)amino]-5-amino-4-(3-chloro-5-methoxyphenyl)thiopheno[2,3-d]pyrimidine-6-carboxamide. 5-amino-4-(3-chloro-5-methoxyphenyl)-2-(methylsulfinyl)thiopheno[2,3-d]pyrimidine-6-carboxamide (500 mg, 1.26 mmol) was dissolved in DMF (4 mL) (2R)-2-aminobutan-1-ol (450 mg, 5.05 mmol) was added. The reaction proceeded under microwave irradiation at 170° C. for 7 minutes. The residue was extracted with EtOAc and washed with 1N HCl. The organic layer was dried with sodium sulfate and concentrated on a rotary evaporator to give 300 mg (56%) of the title compound as a yellow oil. MS m/z calculated for (M+H)⁺ 422, found 422.

Step b: 2-[((1S)-1-ethyl-2-hydroxyethyl)amino]-5-amino-4-(5-chloro-3-hydroxyphenyl)thiopheno[2,3-d]pyrimidine-6-carboxamide. 2-[((1S)-1-ethyl-2-hydroxyethyl)amino]-5-amino-4-(3-chloro-5-methoxyphenyl)thiopheno[2,3-d]pyrimidine-6-carboxamide (300 mg, 713 mmol) was dissolved in CH₂Cl₂ (20 mL) and BBr₃ (3.56 mL) was added. The reaction was stirred at room temperature for 18 h when an additional 5 mL of BBr₃ was added. After an additional 5 h, the mixture was added to ice and neutralized with saturated NaHCO₃. The aqueous layer was extracted with ethyl acetate, dried with sodium sulfate, and concentrated. The residue was purified using reverse phase preparatory HPLC (10-65% CH₃CN/H₂O), then re-purified using reverse phase semipreperative HPLC (5-55% CH₃CN/H₂O). CH₃CN was removed on a rotary evaporator. The resulting aqueous mixture was extracted with EtOAc and the organic layer was washed 5 times with saturated NaHCO₃ to remove TFA. The organic layer was dried with sodium sulfate and concentrated on a rotary evaporator. The resulting residue was triturated with EtOAc/Hexane and the yellow powder filtered and dried under high vacuum to give 10 mg (3%) of the title compound as a yellow solid. 1H NMR (400 MHz, DMSO-D6) δ ppm 0.89 (t, J=8.20 Hz, 3H) 1.15-1.31 (m, 2H) 3.47 (s, 1H) 3.90 (s, 1H) 4.66 (s, 1H) 6.07 (s, 2H) 6.88 (s, 2H) 6.98 (s, 1H) 7.03 (s, 1H) 7.50 (d, J=9.37 Hz, 2H). MS m/z calculated for (M+H)⁺ 408, found 408.

### 5.2.68 2-[((1R)-1-ethyl-2-hydroxyethyl)amino]-5-amino-4-(5-chloro-3-hydroxyphenyl)thiopheno[2,3-d]pyrimidine-6-carboxamide

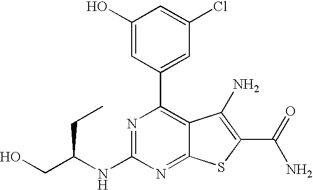

Step a: 2-[((1R)-1-ethyl-2-hydroxyethyl)amino]-5-amino-4-(3-chloro-5-methoxyphenyl)thiopheno[2,3-d]pyrimidine-6-carboxamide. 5-amino-4-(3-chloro-5-methoxyphenyl)-2-(methylsulfinyl)thiopheno[2,3-d]pyrimidine-6-carboxamide (250 mg, 631 mmol) was dissolved in DMF (4 mL) (2S)-2-aminobutan-1-ol (225 mg, 2.52 mmol) was added. The reaction proceeded under microwave irradiation at 170° C. for 7 minutes. The residue was extracted with EtOAc and washed with 1N HCl. The organic layer was dried with sodium sulfate and concentrated on a rotary evaporator to give 200 mg (70%) of the title compound as a yellow oil. MS m/z calculated for (M+H)⁺ 422, found 422.

Step b: 2-[((1R)-1-ethyl-2-hydroxyethyl)amino]-5-amino-4-(5-chloro-3-hydroxyphenyl)thiopheno[2,3-d]pyrimidine-6-carboxamide. 2-[((1R)-1-ethyl-2-hydroxyethyl)amino]-5-amino-4-(3-chloro-5-methoxyphenyl)thiopheno[2,3-d]pyrimidine-6-carboxamide (200 mg, 475 mmol) was dissolved in CH₂Cl₂ (20 mL) and BBr₃ (3.56 mL) was added. The reaction was stirred at room temperature for 18 h when an additional 5 mL of BBr₃ was added. After an additional 5 h, the mixture was added to ice and neutralized with saturated NaHCO₃. The aqueous layer was extracted with ethyl acetate, dried with sodium sulfate, and concentrated. The residue was purified using reverse phase preparatory HPLC (5-60% CH₃CN/H₂O), then re-purified using reverse phase semipreperative HPLC (5-55% CH₃CN/H₂O). CH₃CN was removed on a rotary evaporator. The resulting aqueous mixture was extracted with EtOAc and the organic layer was washed 5 times with saturated NaHCO₃ to remove TFA. The organic layer was dried with sodium sulfate and concentrated on a rotary evaporator. The resulting residue was triturated with EtOAc/Hexane and the yellow powder filtered and dried under high vacuum to give 5 mg (3%) of the title compound as a yellow solid. ¹H NMR (400 MHz, DMSO-D6) δ ppm 0.89 (t, J=8.20 Hz, 3H) 1.15-1.31 (m, 2H) 3.47 (s, 1H) 3.90 (s, 1H) 4.66 (s, 1H) 6.07 (s, 2H) 6.88 (s, 2H) 6.98 (s, 1H) 7.03 (s, 1H) 7.50 (d, J=9.37 Hz, 2H). MS m/z calculated for (M+H)⁺ 408, found 408.

### 5.2.69 5-amino-4-(3,4-dichlorophenyl)thiopheno[2,3-d]pyrimidine-2,6-dicarboxamide

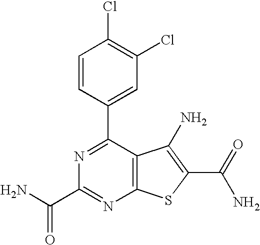

5-amino-4-(3,4-dichlorophenyl)thiopheno[2,3-d]pyrimidine-2,6-dicarboxamide. Suspended 5-amino-4-(3,4-dichlorophenyl)-2-cyanothiopheno[2,3-d]pyrimidine-6-carboxamide (850 mg, 2.33 mmol) in ethanol (50 mL), added 2.0 N NaOH (2.38 mL) and H₂O₂ (0.85 mL) and allowed to stir at room temp for 1.5 hours. Filtered precipitate and washed with ethanol. Purified via prep HPLC (20-100% MeCN/H₂O 30 minutes) to give 15 mg as an orange solid. ¹H NMR (400 MHz, D6-DMSO) δ ppm 6.41 (s, 2H) 7.53 (s, 2H) 7.74 (d, J=8.1 Hz, 1H) 7.84 (d, J=8.1 Hz, 1H) 7.92 (s, 1H) 8.10 (s, 1H) 8.40 (s, 1H). MS m/z calculated for (M+H)⁺ 383, found 383. Analytical HPLC retention time: 4.58 minutes (method I).

### 5.2.70 5-amino-4-(3,4-dichlorophenyl)thiopheno[2,3-d]pyrimidine-6-carboxamide

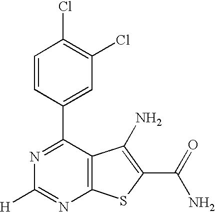

Step a: 6-(3,4-dichlorophenyl)-4-hydroxypyrimidine-5-carbonitrile. Placed 3,4-dichlorobenzaldehyde (8.0 g, 45.7 mmol), ethylcyanoacetate (5.4 mL, 50.3 mmol), formamidine (6.2 g, 59.4 mmol), potassium carbonate (18.9 g, 137.1 mmol), and ethanol (160 mL) in a round-bottomed flask and heated to 80° C. overnight. Filtered precipitate and washed with ethanol. Suspended the precipitate in water and filtered. Dried via toluene azeotrope to give 6.25 g as a white solid. ¹H NMR (400 MHz, D6-DMSO) δ ppm 7.45 (d, J=8.4 Hz, 1H) 7.79 (dd, J=2.0, 8.4 Hz, 1H) 7.99 (d, J=2.0 Hz, 1H) 8.24 (s, 1H). MS m/z calculated for (M+H)⁺ 266, found 266.

Step b: 6-(3,4-dichlorophenyl)-4-chloropyrimidine-5-carbonitrile. Dissolved 6-(3,4-dichlorophenyl)-4-hydroxypyrimidine-5-carbonitrile (6.25 g, 23.4 mmol) in anhydrous 1,4-dioxane. Added POCl₃ (25 mL) and heated to 100° C. overnight. Poured reaction into ice, neutralized with potassium carbonate, extracted with ethyl acetate (2×250 mL), dried with MgSO₄, and concentrated in vacuo to give 4.71 g as a white solid. ¹H NMR (400 MHz, D6-DMSO) δ ppm 7.95 (d, J=8.4 Hz, 1H) 7.99 (dd, J=2.0, 8.4 Hz, 1H) 8.22 (d, J=2.0 Hz, 1H) 9.36 (s, 1H). MS m/z calculated for (M+H)⁺ 285, found 285.

Step c: 2-[6-(3,4-dichlorophenyl)-5-cyanopyrimidin-4-ylthio]acetamide. Dissolved 6-(3,4-dichlorophenyl)-4-chloropyrimidine-5-carbonitrile (4.51 g, 15.8 mmol), 2-sulfanylacetamide (1.44 g, 15.8 mmol), and diisopropylethyl amine (4.1 mL, 23.7 mmol) in dichloromethane (40 mL) and ethanol (40 mL) and allowed to stir at room temperature for 1.5 hours. Filtered precipitate and washed with cold ethanol to give 2.72 g as a white solid. ¹H NMR (400 MHz, D6-DMSO) δ ppm 4.11 (s, 2H) 7.29 (s, 1H) 7.71 (s, 1H) 7.91 (d, J=8.4 Hz, 1H) 7.95 (dd, J=2.0, 8.4 Hz, 1H) 8.20 (d, J=2.0 Hz, 1H) 9.19 (s, 1H). MS m/z calculated for (M+H)⁺ 339, found 339.

Step d: 5-amino-4-(3,4-dichlorophenyl)thiopheno[2,3-d]pyrimidine-6-carboxamide. Prepared a sodium ethoxide solution (203 mg Na, 10 mL EtOH) added 2-[6-(3,4-dichlorophenyl)-5-cyanopyrimidin-4-ylthio]acetamide (1.0 g, 2.95 mmol) and allowed to stir at room temperature for 2.5 hours. Filtered precipitate and washed with cold ethanol. Purified 295 mg via prep HPLC (20-100% MeCN/H₂O 30 minutes) to give 63 mg as a yellow solid. ¹H NMR (400 MHz, D6-DMSO) δ ppm 6.34 (s, 2H) 7.44 (s, 2H) 7.68 (dd, J=2.4, 8.4 Hz, 1H) 7.84 (d, J=8.4 Hz, 1H), 7.95 (d, J=2.4 Hz, 1H) 9.15 (s, 1H). MS m/z calculated for (M+H)⁺ 340, found 340. Analytical HPLC retention time: 5.28 minutes (method I).

### 5.2.71 5-amino-4-(3,4-dichlorophenyl)-2-methylthiopheno[2,3-d]pyrimidine-6-carboxamide

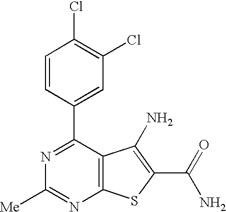

Step a: 6-(3,4-dichlorophenyl)-4-hydroxy-2-methylpyrimidine-5-carbonitrile. Placed 3,4-dichlorobenzaldehyde (8.0 g, 45.7 mmol), ethylcyanoacetate (5.4 mL, 50.3 mmol), acetamidine (5.6 g, 59.4 mmol), potassium carbonate (18.9 g, 137.1 mmol), and ethanol (160 mL) in a round-bottomed flask and heated to 80° C. overnight. Filtered precipitate and washed with ethanol. Suspended the precipitate in water and filtered. Dried via toluene azeotrope to give 10.2 g as a white solid. ¹H NMR (400 MHz, D6-DMSO) δ ppm 2.21 (s, 3H) 7.73 (d, J=8.4 Hz, 1H) 7.78 (dd, J=2.0, 8.4 Hz, 1H) 7.95 (d, J=2.0 Hz, 1H). MS m/z calculated for (M+H)⁺ 280, found 280.

Step b: 6-(3,4-dichlorophenyl)-4-chloro-2-methylpyrimidine-5-carbonitrile. Dissolved 6-(3,4-dichlorophenyl)-4-hydroxy-2-methylpyrimidine-5-carbonitrile (6.15 g, 22.0 mmol) in anhydrous 1,4-dioxane. Added POCl₃ (25 mL) and heated to 100° C. overnight. Poured reaction into ice, neutralized with potassium carbonate, extracted with ethyl acetate (2×250 mL), dried with MgSO₄, and concentrated in vacuo to give 4.71 g as a white solid. ¹H NMR (400 MHz, D6-DMSO) δ ppm 2.78 (s, 3H) 7.93 (d, J=8.2 Hz, 1H) 7.96 (dd, J=2.2, 8.2 Hz, 1H) 8.20 (d, J=2.2 Hz, 1H). MS m/z calculated for (M+H)⁺ 298, found 298.

Step c: 2-[6-(3,4-dichlorophenyl)-5-cyano-2-methylpyrimidin-4-ylthio]acetamide. Dissolved 6-(3,4-dichlorophenyl)-4-chloro-2-methylpyrimidine-5-carbonitrile (3.98 g, 13.3 mmol), 2-sulfanylacetamide (1.21 g, 13.3 mmol), and diisopropylethyl amine (3.5 mL, 20.0 mmol) in dichloromethane (40 mL) and ethanol (40 mL) and allowed to stir at room temperature for 1.5 hours. Filtered precipitate and washed with cold ethanol to give 2.57 g as a white solid. ¹H NMR (400 MHz, D6-DMSO) δ ppm 2.70 (s, 3H) 4.08 (s, 2H) 7.27 (s, 2H) 7.71 (s, 2H) 7.89 (d, J=8.4 Hz, 1H) 7.92 (dd, J=2.0, 8.4 Hz, 1H) 8.16 (d, J=2.0 Hz, 1H). MS m/z calculated for (M+H)⁺ 353, found 353.

Step d: 5-amino-4-(3,4-dichlorophenyl)-2-methylthiopheno[2,3-d]pyrimidine-6-carboxamide. Prepared a sodium ethoxide solution (195 mg Na, 10 mL EtOH) added 2-[6-(3,4-dichlorophenyl)-5-cyano-2-methylpyrimidin-4-ylthio]acetamide (1.0 g, 2.83 mmol) and allowed to stir at room temperature for 2.5 hours. Filtered precipitate and washed with cold ethanol. Purified 500 mg via prep HPLC (20-100% MeCN/H₂O 30 minutes) to give 134 mg as a yellow solid. ¹H NMR (400 MHz, D6-DMSO) δ ppm 2.73 (s, 3H) 6.30 (s, 2H) 7.36 (s, 2H) 7.64 (dd, J=2.4, 8.4 Hz, 1H) 7.82 (d, J=8.4 Hz, 1H) 7.92 (d, J=2.4 Hz, 1H). MS m/z calculated for (M+H)⁺ 353, found 353. Analytical HPLC retention time: 5.44 minutes (method I).

### 5.2.72 5-amino-4-(3,4-dichlorophenyl)-2-(2-methylpropyl)thiopheno[2,3-d]pyrimidine-6-carboxamide

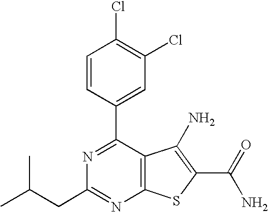

Step a: ethyl (2E)-3-(3,4-dichlorophenyl)-2-cyanoprop-2-enoate. Dissolved 3,4-dichlorobenzaldehyde (4.5 g, 25.7 mmol), ethylcyanoacetate (2.75 mL, 25.7 mmol), and piperidine (2 drops) in toluene. Heated to 135° C. in a Dean-Stark trap to drive off water. Removed solvent in vacuo to give 5.4 g as a yellow solid. ¹H NMR (400 MHz, D6-DMSO) δ ppm 1.31 (t, 3H) 4.33 (q, 2H) 7.90 (d, J=8.4 Hz, 1H) 8.06 (dd, J=2.4, 8.4 Hz, 1H) 8.30 (d, J=2.4 Hz, 1H) 8.43 (s, 1H).

Step b: 6-(3,4-dichlorophenyl)-4-hydroxy-2-(2-methylpropyl)pyrimidine-5-carbonitrile. Placed ethyl (2E)-3-(3,4-dichlorophenyl)-2-cyanoprop-2-enoate (2.0 g, 7.40 mmol), 3-methylbutanamidine (1.48 g, 14.8 mmol), potassium carbonate (3.06 g, 22.2 mmol), and ethanol (50 mL) in a round-bottomed flask and heated to 80° C. overnight. Filtered precipitate and washed with ethanol. Suspended the precipitate in water and filtered. Dried via toluene azeotrope to give 1.50 g as a white solid. MS m/z calculated for (M+H)⁺ 322, found 322.

Step c: 6-(3,4-dichlorophenyl)-4-chloro-2-(2-methylpropyl)pyrimidine-5-carbonitrile. Dissolved 6-(3,4-dichlorophenyl)-4-hydroxy-2-(2-methylpropyl)pyrimidine-5-carbonitrile (1.50 g, 7.40 mmol) in anhydrous 1,4-dioxane. Added POCl₃ (8 mL) and heated to 100° C. overnight. Poured reaction into ice, neutralized with potassium carbonate, extracted with ethyl acetate (2×150 mL), dried with MgSO₄, and concentrated in vacuo to give 733 mg as a yellow solid. MS m/z calculated for (M+H)⁺ 340, found 340.

Step d: 2-[6-(3,4-dichlorophenyl)-5-cyano-2-(2-methylpropyl)pyrimidin-4-ylthio]acetamide. Dissolved 6-(3,4-dichlorophenyl)-4-chloro-2-(2-methylpropyl)pyrimidine-5-carbonitrile (0.73 g, 2.16 mmol), 2-sulfanylacetamide (196 mg, 2.16 mmol), and diisopropylethyl amine (0.6 mL, 3.24 mmol) in dichloromethane (8 mL) and ethanol (8 mL) and allowed to stir at room temperature for 1.5 hours. Filtered precipitate and washed with cold ethanol to give 570 mg as a white solid. MS m/z calculated for (M+H)⁺ 395, found 395.

Step e: 5-amino-4-(3,4-dichlorophenyl)-2-(2-methylpropyl)thiopheno[2,3-d]pyrimidine-6-carboxamide. Prepared a sodium ethoxide solution (100 mg Na, 10 mL EtOH) added 2-[6-(3,4-dichlorophenyl)-5-cyano-2-(2-methylpropyl)pyrimidin-4-ylthio]acetamide (0.57 g, 1.44 mmol) and allowed to stir at room temperature for 2.5 hours. Filtered precipitate and washed with cold ethanol. Purified via prep HPLC (20-100% MeCN/H₂O 30 minutes) to give 220 mg as a yellow solid. ¹H NMR (400 MHz, D6-DMSO) δ ppm MS m/z calculated for (M+H)⁺ 395, found 395. Analytical HPLC retention time: 6.56 minutes (method I).

### 5.2.73 2-{[(1S)-2-hydroxy-1-(methylethyl)ethyl]amino}-5-amino-4-(3,4-dichlorophenyl)thiopheno[2,3-d]pyrimidine-6-carboxamide

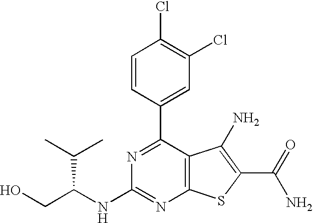

2-{[(1S)-2-hydroxy-1-(methylethyl)ethyl]amino}-5-amino-4-(3,4-dichlorophenyl)thiopheno[2,3-d]pyrimidine-6-carboxamide. 5-amino-4-(3,4-dichlorophenyl)-2-(methylsulfinyl)thiopheno[2,3-d]pyrimidine-6-carboxamide (360 mg, 0.90 mmol), (2S)-2-amino-3-methylbutan-1-ol (464 mg, 4.50 mmol), and anhydrous THF (15 mL) were placed in a round-bottomed flask and heated to 75° C. overnight. The volatiles were removed in vacuo, the residue suspended in ethanol, and the precipitate collected via vacuum filtration to give 170 mg as a yellow solid. Purified via prep HPLC (20-100% MeCN/H₂O 30 minutes) to give 60 mg as a yellow solid. ¹H NMR (400 MHz, D6-DMSO) δ ppm 0.91 (s, 6H) 1.96 (m, 1H) 1.99 (s, 3H) 3.50 (m, 2H) 4.55 (m, 2H) 6.14 (s, 2H) 7.00 (s, 2H) 7.50 (m, 2H) 7.80 (m, 2H). MS m/z calculated for (M+H)⁺ 440, found 440. Analytical HPLC retention time: 5.60 minutes (method I).

### 5.2.74 2-{[(1R)-2-hydroxy-1-(methylethyl)ethyl]amino}-5-amino-4-(3,4-dichlorophenyl)thiopheno[2,3-d]pyrimidine-6-carboxamide

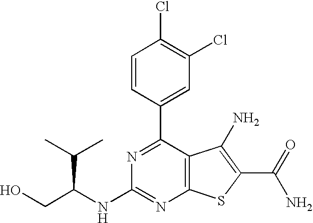

2-{[(1R)-2-hydroxy-1-(methylethyl)ethyl]amino}-5-amino-4-(3,4-dichlorophenyl)thiopheno[2,3-d]pyrimidine-6-carboxamide. 5-amino-4-(3,4-dichlorophenyl)-2-(methylsulfinyl)thiopheno[2,3-d]pyrimidine-6-carboxamide (360 mg, 0.90 mmol), (2S)-2-amino-3-methylbutan-1-ol (464 mg, 4.50 mmol), and anhydrous THF (15 mL) were placed in a round-bottomed flask and heated to 75° C. overnight. The volatiles were removed in vacuo, the residue suspended in ethanol, and the precipitate collected via vacuum filtration to give 150 mg as a yellow solid. Purified via prep HPLC (20-100% MeCN/H₂O 30 minutes) to give 100 mg as a yellow solid. ¹H NMR (400 MHz, D6-DMSO) δ ppm 0.91 (s, 6H) 1.96 (m, 1H) 1.99 (s, 3H) 3.50 (m, 2H) 4.55 (m, 2H) 6.14 (s, 2H) 7.00 (s, 2H) 7.50 (m, 2H) 7.80 (m, 2H). MS m/z calculated for (M+H)⁺ 440, found 440. Analytical HPLC retention time: 5.60 minutes (method I).

### 5.2.75 5-amino-2-[(2-hydroxy-tert-butyl)amino]-4-(3-hydroxy-4-methylphenyl)thiopheno[2,3-d]pyrimidine-6-carboxamide

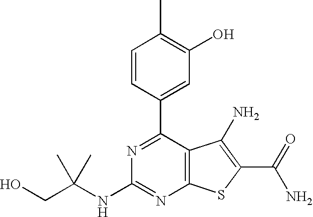

5-amino-2-[(2-hydroxy-tert-butyl)amino]-4-(3-hydroxy-4-methylphenyl)thiopheno[2,3-d]pyrimidine-6-carboxamide. Dissolved 5-amino-2-[(2-hydroxy-tert-butyl)amino]-4-(3-methoxy-4-methylphenyl)thiopheno[2,3-d]pyrimidine-6-carboxamide (110 mg, 0.27 mmol) in dichloromethane (4 mL) and added 1.0 M BBr₃ (1.08 mL, 1.08 mmol) and allowed to stir at room temp for 4 hours. Poured into water and separated layers. Adjusted pH=6, and extracted with ethyl acetate (3×50 mL), washed with brine (50 mL), dried with sodium sulfate. Purified via prep HPLC (20-80% MeCN/H₂O 30 minutes) to give 30 mg as a yellow solid. ¹H NMR (400 MHz, D6-DMSO) δ ppm 1.33 (s, 6H) 2.20 (s, 3H) 3.54 (d, J=6.0 Hz, 2H) 4.86 (t, J=6.0 Hz, 1H) 6.10 (s, 2H) 6.95 (m, 5H) 7.23 (d, J=8.0 Hz, 1H) 9.75 (s, 1H). MS m/z calculated for (M+H)⁺ 388, found 388. Analytical HPLC retention time: 4.62 minutes (method I).

### 5.2.76 2-[((1S)-2-hydroxy-isopropyl)amino]-5-amino-4-(3,4-dichlorophenyl)thiopheno[2,3-d]pyrimidine-6-carboxamide

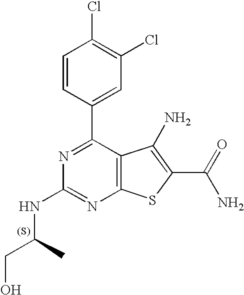

5-amino-4-(3,4-dichlorophenyl)-2-(methylsulfinyl)thiopheno[2,3-d]pyrimidine-6-carboxamide (200 mg, 0.5 mmol) and (S)-(+)-2-amino-1-propanol (113 mg, 1.5 mmol) were combined and heated at 90° C. for 18 h without solvent. The crude product was dissolved in DMSO (2 mL) and purified using reverse-phase preparative HPLC (30-80% acetonitrile, 0.1% TFA, 30 minutes). The appropriate fractions were combined and concentrated on a rotary evaporator. The residue was dissolved in ethyl acetate (100 mL), washed with aqueous K₂CO₃, and dried over Na₂SO₄. The solvent was removed in vacuo to give 35 mg (17%) of product as a light yellow solid. ¹H NMR (400 MHz, D6-DMSO) δ ppm 7.85 (d, 1H, J=2 Hz), 7.79 (d, 1H, J=8), 7.58 (d, 1H, J=8 Hz), 7.01 (s, 2H), 6.15 (bs, 2H), 4.71 (m, 1H), 4.05 (m, 1H), 3.47 (m, 1H), 3.32 (m, 1H), 1.14 (d, 3H, J=7 Hz). MS m/z calculated for (M+H)⁺ 412, found 412.

### 5.2.77 2-[((1R)-2-hydroxy-isopropyl)amino]-5-amino-4-(3,4-dichlorophenyl)thiopheno[2,3-d]pyrimidine-6-carboxamide

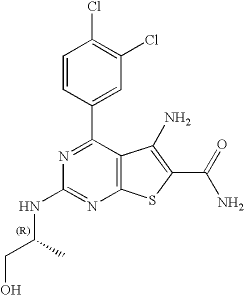

5-amino-4-(3,4-dichlorophenyl)-2-(methylsulfinyl)thiopheno[2,3-d]pyrimidine-6-carboxamide (200 mg, 0.5 mmol) and (R)-(−)-2-amino-1-propanol (113 mg, 1.5 mmol) were combined and heated at 90° C. for 18 h without solvent. The crude product was dissolved in DMSO (2 mL) and purified using reverse-phase preparative HPLC (30-80% acetonitrile, 0.1% TFA, 30 minutes). The appropriate fractions were combined and concentrated on a rotary evaporator. The residue was dissolved in ethyl acetate (100 mL), washed with aqueous K₂CO₃, and dried over Na₂SO₄. The solvent was removed in vacuo to give 28 mg (13.6%) of product as a light yellow solid. ¹H NMR (400 MHz, D6-DMSO) δ ppm 7.85 (d, 1H, J=2 Hz), 7.79 (d, 1H, J=8 Hz), 7.58 (d, 1H, J=8 Hz), 7.00 (s, 2H), 6.14 (bs, 2H), 4.70 (m, 1H), 4.05 (m, 1H), 3.48 (m, 1H), 3.32 (m, 1H), 1.15 (d, 3H, J=7 Hz). MS m/z calculated for (M+H)⁺ 412, found 412.

### 5.2.78 5-amino-4-(3,4-dichlorophenyl)-2-[(2-carbamoylethyl)amino]thiopheno[2,3-d]pyrimidine-6-carboxamide

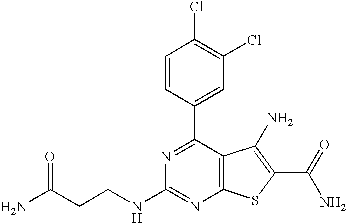

5-amino-4-(3,4-dichlorophenyl)-2-(methylsulfinyl)thiopheno[2,3-d]pyrimidine-6-carboxamide (160 mg, 0.4 mmol), 3-aminopropanamide hydrochloride (232 mg, 1.8 mmol), DIEA (0.5 mL) and DMF (1.5 mL) were combined in a glass vial and heated at 90° C. for 18 hours. The reaction mixture was diluted with DMSO (2 mL) and the product was purified using reverse-phase preparative HPLC (30-80% acetonitrile, 0.1% TFA, 30 min). The appropriate fractions were combined and concentrated on a rotary evaporator. The residue was dissolved in ethyl acetate (100 mL), washed with aqueous K₂CO₃, and dried over Na₂SO₄. The solvent was removed in vacuo to give 30 mg (17%) of product as a light yellow solid. ¹H NMR (400 MHz, D6-DMSO) δ ppm 7.85 (m, 1H), 7.79 (d, 1H, J=8 Hz), 7.57 (m, 1H), 7.03 (s, 2H), 6.85 (m, 1H), 6.15 (bs, 2H), 3.52 (m, 2H), 2.38 (m, 2H). MS m/z calculated for (M+H)⁺ 439, found 439.

### 5.2.79 5-amino-4-(3,4-dichlorophenyl)-2-[(2-hydroxy-tert-butyl)amino]thiopheno[2,3-d]pyrimidine-6-carboxamide

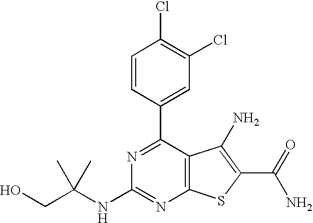

5-amino-4-(3,4-dichlorophenyl)-2-(methylsulfinyl)thiopheno[2,3-d]pyrimidine-6-carboxamide (150 mg, 0.375 mmol), 2-amino-2-methyl-1-propanol (450 mg, 5.05 mmol), and DMF (1.0 mL) were combined in a glass vial and heated at 90° C. for 18 hours. The reaction mixture was diluted with DMSO (1 mL) and the product was purified using reverse-phase preparative HPLC (30-80% acetonitrile, 0.1% TFA, 30 minutes). The appropriate fractions were combined and concentrated on a rotary evaporator. The residue was dissolved in ethyl acetate (100 mL), washed with aqueous K₂CO₃, and dried over Na₂SO₄. The solvent was removed in vacuo to give 33.5 mg (21%) of product as a light yellow solid. ¹H NMR (400 MHz, D6-DMSO) δ ppm 7.85 (d, 1H, J=2.4 Hz), 7.79 (d, 1H, J=8 Hz), 7.59 (d, 1H, J=8 Hz), 7.12 (s, 1H), 7.03 (s, 2H), 6.18 (bs, 2H), 4.84 (t, 1H, J=8), 3.50 (d, 2H, J=8 Hz), 1.34 (s, 6H). MS m/z calculated for (M+H)⁺ 426, found 426.

### 5.2.80 5-amino-4-(3,4-dichlorophenyl)-2-{[2-hydroxy-1-(hydroxymethyl)ethyl]amino}thiopheno[2,3-d]pyrimidine-6-carboxamide

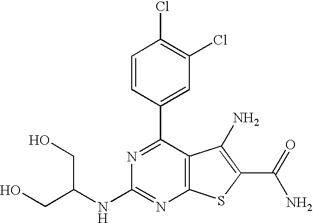

5-amino-4-(3,4-dichlorophenyl)-2-(methylsulfinyl)thiopheno[2,3-d]pyrimidine-6-carboxamide (300 mg, 0.75 mmol), 2-aminopropane-1,3-diol (273 mg, 3.0 mmol), and DMSO (1.0 mL) were combined in a glass vial and heated at 90° C. for 18 hours. The reaction mixture was diluted with DMSO (1 mL) and the product was purified using reverse-phase preparative HPLC (30-80% acetonitrile, 0.1% TFA, 30 minutes). The appropriate fractions were combined and concentrated on a rotary evaporator. The residue was dissolved in ethyl acetate (100 mL), washed with aqueous K₂CO₃, and dried over Na₂SO₄. The solvent was removed in vacuo to give 100 mg (31%) of product as a light yellow solid. ¹H NMR (400 MHz, D6-DMSO) δ ppm 7.86 (d, 1H, J=2.4 Hz), 7.79 (d, 1H, J=10 Hz), 7.59 (m, 1H,), 7.03 (s, 2H), 6.16 (bs, 2H), 4.66 (bs, 2H), 3.51 (m, 4H), 3.31 (m, 1H). MS m/z calculated for (M+H)⁺ 428, found 428.

### 5.2.81 2-[((2S)-2-hydroxypropyl)amino]-5-amino-4-(3,4-dichlorophenyl)thiopheno[2,3-d]pyrimidine-6-carboxamide

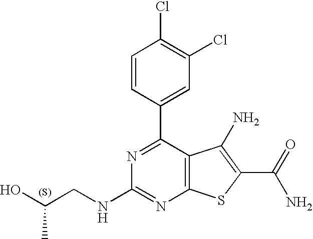

5-amino-4-(3,4-dichlorophenyl)-2-(methylsulfinyl)thiopheno[2,3-d]pyrimidine-6-carboxamide (250 mg, 0.625 mmol), (S)-(+)-1-amino-2-propanol (188 mg, 2.5 mmol), and DMSO (1.0 mL) were combined in a glass vial and heated at 90° C. for 18 hours. The reaction mixture was diluted with DMSO (1 mL) and the product was purified using reverse-phase preparative HPLC (30-80% acetonitrile, 0.1% TFA, 30 min). The appropriate fractions were combined and concentrated on a rotary evaporator. The residue was dissolved in ethyl acetate (100 mL), washed with aqueous K₂CO₃, and dried over Na₂SO₄. The solvent was removed in vacuo to give 41 mg (16%) of product as a light yellow solid. ¹H NMR (400 MHz, D6-DMSO) δ ppm 7.85 (d, 1H, J=1.6 Hz), 7.79 (d, 1H, J=8.4 Hz), 7.58 (m, 1H), 7.02 (s, 2H), 6.14 (bs, 2H), 4.71 (m, 1H), 3.82 (m, 1H), 3.25 (m, 1H), 1.08 (d, 3H, J=4.8 Hz). MS m/z/z calculated for (M+H)⁺ 412, found 412.

### 5.2.82 2-[((2R)-2-hydroxypropyl)amino]-5-amino-4-(3,4-dichlorophenyl)thiopheno[2,3-d]pyrimidine-6-carboxamide

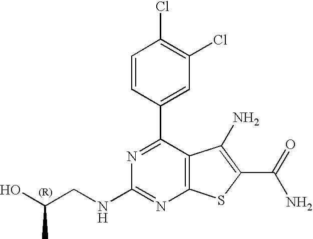

5-amino-4-(3,4-dichlorophenyl)-2-(methylsulfinyl)thiopheno[2,3-d]pyrimidine-6-carboxamide (250 mg, 0.625 mmol), (R)-(−)-1-amino-2-propanol (188 mg, 2.5 mmol), and DMSO (1.0 mL) were combined in a glass vial and heated at 90° C. for 18 hours. The reaction mixture was diluted with DMSO (1 mL) and the product was purified using reverse-phase preparative HPLC (30-80% acetonitrile, 0.1% TFA, 30 min). The appropriate fractions were combined and concentrated on a rotary evaporator. The residue was dissolved in ethyl acetate (100 mL), washed with aqueous K₂CO₃, and dried over Na₂SO₄. The solvent was removed in vacuo to give 43 mg (16.7%) of product as a light yellow solid. ¹H NMR (400 MHz, D6-DMSO) δ ppm 7.85 (d, 1H, J=1.6 Hz), 7.79 (d, 1H, J=8.4 Hz), 7.58 (m, 1H), 7.02 (s, 2H), 6.14 (bs, 2H), 4.71 (m, 1H), 3.82 (m, 1H), 3.25 (m, 1H), 1.08 (d, 3H, J=4.8 Hz). MS m/z calculated for (M+H)⁺ 412, found 412.

### 5.2.83 2-[((1S)-1-ethyl-2-hydroxyethyl)amino]-5-amino-4-(3,4-dichlorophenyl)thiopheno[2,3-d]pyrimidine-6-carboxamide

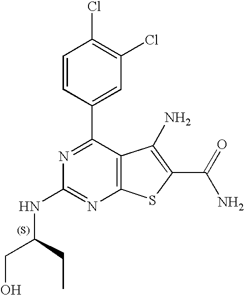

5-amino-4-(3,4-dichlorophenyl)-2-(methylsulfinyl)thiopheno[2,3-d]pyrimidine-6-carboxamide (250 mg, 0.625 mmol), (S)-(+)-2-amino-1-butanol (222 mg, 2.5 mmol), and DMSO (1.0 mL) were combined in a glass vial and heated at 90° C. for 18 hours. The reaction mixture was diluted with DMSO (1 mL) and the product was purified using reverse-phase preparative HPLC (30-80% acetonitrile, 0.1% TFA, 30 minutes). The appropriate fractions were combined and concentrated on a rotary evaporator. The residue was dissolved in ethyl acetate (100 mL), washed with aqueous K₂CO₃, and dried over Na₂SO₄. The solvent was removed in vacuo to give 23.2 mg (8.7%) of product as a light yellow solid. ¹H NMR (400 MHz, D6-DMSO) δ ppm 7.86 (m, 1H), 7.79 (d, 1H, J=8.4 Hz), 7.58 (m, 1H), 7.01 (s, 2H), 6.16 (bs, 2H), 4.66 (m, 1H), 3.91 (m, 1H), 3.47 (m, 1H), 1.68 (m, 1H), 1.44 (m, 1H), 0.88 (t, 3H, J=7.2 Hz). MS m/z calculated for (M+H)⁺ 426, found 426.

### 5.2.84 2-[((1R)-1-ethyl-2-hydroxyethyl)amino]-5-amino-4-(3,4-dichlorophenyl)thiopheno[2,3-d]pyrimidine-6-carboxamide

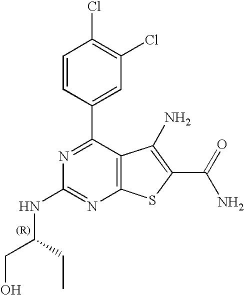

5-amino-4-(3,4-dichlorophenyl)-2-(methylsulfinyl)thiopheno[2,3-d]pyrimidine-6-carboxamide (250 mg, 0.625 mmol), (R)-(−)-2-amino-1-butanol (222 mg, 2.5 mmol), and DMSO (1.0 mL) were combined in a glass vial and heated at 90° C. for 18 hours. The reaction mixture was diluted with DMSO (1 mL) and the product was purified using reverse-phase preparative HPLC (30-80% acetonitrile, 0.1% TFA, 30 min). The appropriate fractions were combined and concentrated on a rotary evaporator. The residue was dissolved in ethyl acetate (100 mL), washed with aqueous K₂CO₃, and dried over Na₂SO₄. The solvent was removed in vacuo to give 19.1 mg (7.2%) of product as a light yellow solid. ¹H NMR (400 MHz, D6-DMSO) δ ppm 7.86 (m, 1H), 7.79 (d, 1H, J=8.4 Hz), 7.58 (m, 1H), 7.01 (s, 2H), 6.16 (bs, 2H), 4.66 (m, 1H), 3.91 (m, 1H), 3.47 (m, 1H), 1.68 (m, 1H), 1.44 (m, 1H), 0.88 (t, 3H, J=7.2 Hz). MS m/z calculated for (M+H)⁺ 426, found 426.

### 5.2.85 5-amino-4-(3,4-dichlorophenyl)-2-[(tert-butyl)amino]thiopheno[2,3-d]pyrimidine-6-carboxamide

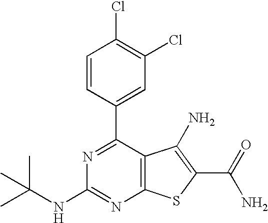

5-amino-4-(3,4-dichlorophenyl)-2-(methylsulfinyl)thiopheno[2,3-d]pyrimidine-6-carboxamide (250 mg, 0.625 mmol), t-butyl amine (457 mg, 6.25 mmol), and DMF (10 mL) were heated at reflux for 16 hours. The solvents were removed in vacuo and the product was purified by silica-gel chromatography (40% hexane in EtOAc) to give 20 mg (8%) of product. ¹H NMR (400 MHz, D6-DMSO) δ ppm 7.86 (d, 1H, J=1.6 Hz), 7.79 (d, 1H, J=8.4 Hz), 7.59 (d, 1H, J=7.6), 7.48 (s, 1H), 7.02 (s, 2H), 6.16 (bs, 2H), 1.40 (s, 9H). MS m/z calculated for (M+H)⁺ 410, found 410.

### 5.2.86 5-amino-4-(3,4-dichlorophenyl)-2-{[(hydroxymethyl)cyclopropyl]amino}thiopheno[2,3-d]pyrimidine-6-carboxamide

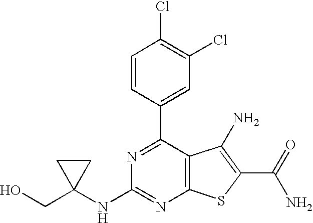

5-amino-4-(3,4-dichlorophenyl)-2-(methylsulfinyl)thiopheno[2,3-d]pyrimidine-6-carboxamide (30 mg, 0.75 mmol), (aminocyclopropyl)methan-1-ol hydrochloride (500 mg, 4.0 mmol, prepared according to Kiely et al. J. Med. Chem. (1988), 31(10), 2004-8.), DIEA (3.0 mL, 18 mmol) and DMF (10 mL) were combined and heated at 90° C. for 18 hours. The solvents were evaporated, the residue was dissolved in DMSO (4 mL), and the product was purified using reverse-phase preparative HPLC (25-60% acetonitrile, 0.1% TFA, 30 min). The appropriate fractions were combined and concentrated on a rotary evaporator. The residue was dissolved in ethyl acetate (100 mL), washed with aqueous NaHCO₃, and dried over Na₂SO₄. The solvent was removed in vacuo to give 35 mg (11%) of product as a light yellow solid. ¹H NMR (400 MHz, D6-DMSO) δ ppm 8.04 (bs, 1H), 7.85 (s, 1H), 7.79 (d, 1H, J=7.6 Hz), 7.58 (m, 1H), 7.04 (s, 2H), 6.14 (bs, 2H), 4.72 (t, 1H, J=6 Hz), 3.58 (m, 2H), 0.80 (m, 2H), 0.67 (m, 2H). MS m/z calculated for (M+H)⁺ 424, found 424.

### 5.2.87 2-((1S)-2-hydroxy-isopropoxy)-5-amino-4-(3,4-dichlorophenyl)thiopheno[2,3-d]pyrimidine-6-carboxamide

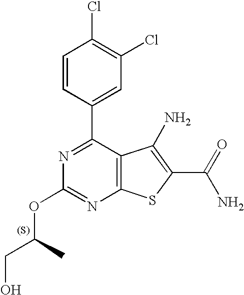

NaH (480 mg, 12 mmol, 60% dispersion in oil) was added at room temperature to a flask containing (S)-(+)-1-Benzyloxy-2-propanol (2.0 g, 12 mmol) in THF (15 mL). After stirring for 30 minutes, 5-amino-4-(3,4-dichlorophenyl)-2-(methylsulfinyl)thiopheno[2,3-d]pyrimidine-6-carboxamide (400 mg, 1 mmol) was added and the mixture was stirred at room temperature for 15 minutes The reaction was quenched with water (10 mL) and the solvent was evaporated. The crude product was passed through a plug of silica gel (eluting with 1:1 hexane/EtOAc), dried over Na₂SO₄, and evaporated to dryness. The crude material (300 mg, 0.6 mmol) was dissolved in DCM (20 mL) and cooled to −45° C. BBr₃ (1.2 mmol) was added and the mixture was stirred for 5 minutes then quenched with MeOH (5 mL). The solvent was removed in vacuo and the residue was dissolved in EtOAc. The organic layer was washed with aqueous NaHCO₃ and dried over Na₂SO₄. The product was purified using reverse-phase preparative HPLC (20-60% acetonitrile, 0.1% TFA, 30 minutes). The appropriate fractions were combined and concentrated on a rotary evaporator. The residue was dissolved in ethyl acetate (100 mL), washed with aqueous NaHCO₃, and dried over Na₂SO₄. The solvent was removed in vacuo to give 10 mg (2.4%) of product as a light yellow solid. ¹H NMR (400 MHz, D6-DMSO) δ ppm 7.93 (d, 1H, J=1.6 Hz), 7.82 (d, 1H, J=8 Hz), 7.65 (dd, 1H, J=2, 8.4 Hz), 7.27 (s, 2H), 6.32 (bs, 2H), 5.23 (m, 1H), 4.93 (t, 1H, J=5.6 Hz), 3.59 (m, 2H), 1.29 (d, 3H, J=6 Hz). MS m/z calculated for (M+H)⁺ 413, found 413.

### 5.2.88 2-((1R)-2-hydroxy-isopropoxy)-5-amino-4-(3,4-dichlorophenyl)thiopheno[2,3-d]pyrimidine-6-carboxamide

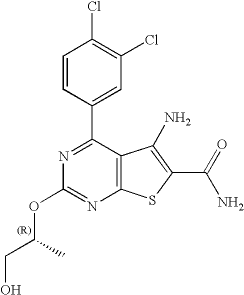

NaH (480 mg, 12 mmol, 60% dispersion in oil) was added at room temperature to a flask containing (R)-(−)-1-Benzyloxy-2-propanol (2.0 g, 12 mmol) in THF (15 mL). After stirring for 30 minutes, 5-amino-4-(3,4-dichlorophenyl)-2-(methylsulfinyl)thiopheno[2,3-d]pyrimidine-6-carboxamide (400 mg, 1 mmol) was added and the mixture was stirred at room temperature for 15 minutes The reaction was quenched with water (10 mL) and the solvent was evaporated. The crude product was passed through a plug of silica-gel (eluting with 1:1 hexane/EtOAc), dried over Na₂SO₄, and evaporated to dryness. The crude material (150 mg, 0.3 mmol) was dissolved in CHCl₃ (10 mL) and cooled to 0° C. TMS-Iodide (0.2 mL, 1 mmol) was added dropwise and the mixture was stirred at 0° C. for 10 minutes followed by room temperature for an additional 10 minutes The reaction was quenched with aqueous NaHCO₃ (3 mL) and the solvents were removed in vacuo. The residue was dissolved in EtOAc and washed with aqueous NaHCO₃ and evaporated. The product was purified by silica-gel chromatography (40% hexane in EtOAc) to give 12 mg (2.9%) of product as a light yellow solid. ¹H NMR (400 MHz, D6-DMSO) δ ppm 7.93 (d, 1H, J=1.6 Hz), 7.82 (d, 1H, J=8 Hz), 7.65 (dd, 1H, J=2, 8.4 Hz), 7.27 (s, 2H), 6.32 (bs, 2H), 5.23 (m, 1H), 4.93 (t, 1H, J=5.6 Hz), 3.59 (m, 2H), 1.29 (d, 3H, J=6 Hz). MS m/z calculated for (M+H)⁺ 413, found 413.

### 5.2.89 2-((1S,2S)-2-hydroxy-1-methylpropoxy)-5-amino-4-(3,4-dichlorophenyl)thiopheno[2,3-d]pyrimidine-6-carboxamide

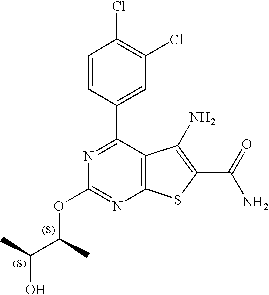

NaH (400 mg, 10 mmol, 60% dispersion in oil) was added at room temperature to a flask containing (2S,3S)-(+)-2,3-butanediol (900 mg, 10 mmol) in THF (40 mL). After stirring for 20 minutes, 5-amino-4-(3,4-dichlorophenyl)-2-(methylsulfinyl)thiopheno[2,3-d]pyrimidine-6-carboxamide (400 mg, 1 mmol) was added and the mixture was stirred at room temperature for 15 minutes The reaction was quenched with water (10 mL) and the solvent was evaporated. The product was purified using silica-gel chromatography (20% hexane in EtOAc) to give 40 mg (9.4%) of the desired product as a light yellow solid. ¹H NMR (400 MHz, D6-DMSO) δ ppm 7.93 (s, 1H), 7.83 (d, 1H, J=8.4 Hz), 7.65 (d, 1H, J=7.6 Hz), 7.27 (s, 2H), 6.31 (bs, 2H), 5.12 (m, 1H), 4.89 (d, 1H, J=4.8 Hz), 3.82 (m, 2H), 1.26 (d, 3H, J=6.4), 1.10 (d, 3H, J=6.4 Hz). MS m/z calculated for (M+H)⁺ 427, found 427.

### 5.2.90 2-((1R,2R)-2-hydroxy-1-methylpropoxy)-5-amino-4-(3,4-dichlorophenyl)thiopheno[2,3-d]pyrimidine-6-carboxamide

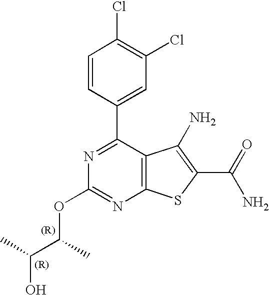

NaH (400 mg, 10 mmol, 60% dispersion in oil) was added at room temperature to a flask containing (2R,3R)-(+)-2,3-butanediol (900 mg, 10 mmol) in THF (40 mL). After stirring for 20 minutes, 5-amino-4-(3,4-dichlorophenyl)-2-(methylsulfinyl)thiopheno[2,3-d]pyrimidine-6-carboxamide (400 mg, 1 mmol) was added and the mixture was stirred at room temperature for 15 minutes The reaction was quenched with water (10 mL) and the solvent was evaporated. The product was purified using silica-gel chromatography (20% hexane in EtOAc) to give 125 mg (29%) of the desired product as a light yellow solid. ¹H NMR (400 MHz, D6-DMSO) δ ppm 7.93 (d, 1H, J=2 Hz), 7.83 (d, 1H, J=8.4 Hz), 7.65 (dd, 1H, J=2, 8 Hz), 7.27 (s, 2H), 6.31 (bs, 2H), 5.12 (m, 1H), 4.89 (d, 1H, J=4.8 Hz), 3.82 (m, 2H), 1.26 (d, 3H, J=6.4), 1.10 (d, 3H, J=6.4 Hz). MS m/z calculated for (M+H)⁺ 427, found 427.

### 5.2.91 5-amino-4-(3,4-dichlorophenyl)-2-[(2-hydroxy-2-methylpropyl)amino]thiopheno[2,3-d]pyrimidine-6-carboxamide

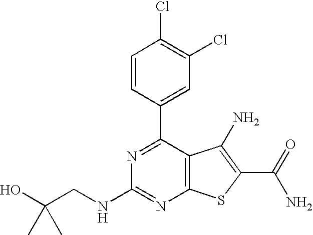

5-amino-4-(3,4-dichlorophenyl)-2-(methylsulfinyl)thiopheno[2,3-d]pyrimidine-6-carboxamide (350 mg, 0.875 mmol), 1-amino-2-methylpropan-2-ol (1.0 g, 11.25 mmol) and ethanol were combined and heated at 65° C. for 18 hours. The crude product was filtered and washed with methanol (10 mL). The crude product (150 mg) was dissolved in DMSO (2 mL) and purified using reverse-phase preparative HPLC (20-80% acetonitrile, 0.1% TFA, 30 minutes). The appropriate fractions were combined and concentrated on a rotary evaporator. The residue was dissolved in ethyl acetate (100 mL), washed with aqueous K₂CO₃, and dried over Na₂SO₄. The solvent was removed in vacuo to give 40 mg (11%) of product as a light yellow solid. ¹H NMR (400 MHz, D6-DMSO) δ ppm 7.86 (s, 1H), 7.79 (d, 1H, J=8 Hz), 7.58 (dd, 1H, J=1.6, 8.4 Hz), 7.02 (s, 2H), 6.15 (bs, 2H), 4.53 (s, 1H), 3.30 (m, 2H), 1.12 (s, 6H). MS m/z calculated for (M+H)⁺ 426, found 426.

### 5.2.92 5-amino-4-(3,4-dichlorophenyl)-2-[(1,1-dimethyl-2-pyrrolidinylethyl)amino]thiopheno[2,3-d]pyrimidine-6-carboxamide

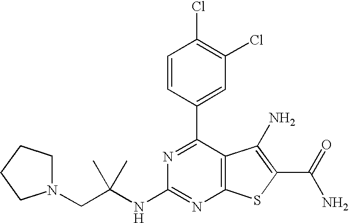

5-amino-4-(3,4-dichlorophenyl)-2-(methylsulfinyl)thiopheno[2,3-d]pyrimidine-6-carboxamide (350 mg, 0.875 mmol 2-methyl-1-pyrrolidinylprop-2-ylamine (1.0 g, 7.0 mmol), and DMF (0.5 mL) were combined in a glass vial and heated at 90° C. for 18 hours. The product was purified using silica-gel chromatography (1% TEA, 10% MeOH, 89% EtOAc) to give 140 mg of semi-pure product as a light yellow solid. Followed by dissolving in EtOAc (5 mL) and precipitation with hexane (100 mL). The product was filtered and dried to give 65 mg (15.5%) as a light yellow solid. ¹H NMR (400 MHz, D6-DMSO) δ ppm 7.86 (d, 1H, J=2 Hz), 7.79 (d, 1H, J=8.4 Hz), 7.59 (d, 1H, J=7.6 Hz), 7.15 (s, 1H), 7.02 (s, 2H), 6.18 (bs, 2H), 2.81 (s, 2H), 2.54 (m, 4H), 1.63 (m, 4H), 1.39 (s, 6H). MS m/z calculated for (M+H)⁺ 479, found 479.

### 5.2.93 5-amino-4-(3,4-dichlorophenyl)-2-(propylamino)thiopheno[2,3-d]pyrimidine-6-carboxamide

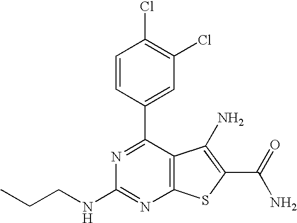

5-amino-4-(3,4-dichlorophenyl)-2-(methylsulfinyl)thiopheno[2,3-d]pyrimidine-6-carboxamide (100 mg, 0.25 mmol), 1-aminopropane (74 mg, 1.25 mmol) and anhydrous THF (1 mL) were placed in a microwave tube and heated to 130° C. for 10 minutes. The volatiles were removed in vacuo, the residue suspended in Ethanol, and the precipitate collected via vacuum filtration to give 28 mg (28%) product as a light yellow solid. ¹H NMR (400 MHz, D6-DMSO) δ ppm 7.85 (m, 1H), 7.78 (m, 1H), 7.58 (m, 1H), 6.99 (s, 2H), 6.13 (bs, 2H), 3.28 (m, 2H), 1.56 (m, 2H), 0.91 (m, 3H). MS m/z calculated for (M+H)⁺ 396, found 396.

### 5.2.94 5-amino-4-(3,4-dichlorophenyl)-2-{[2-(methylamino)ethyl]amino}thiopheno[2,3-d]pyrimidine-6-carboxamide

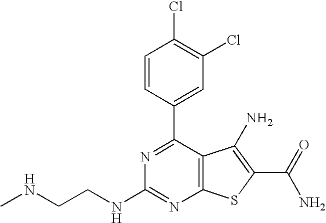

5-amino-4-(3,4-dichlorophenyl)-2-(methylsulfinyl)thiopheno[2,3-d]pyrimidine-6-carboxamide (200 mg, 0.5 mmol) and N-(2-aminoethyl)(tert-butoxy)-N-methylcarboxamide (261 mg, 1.5 mmol) were combined and heated at 90° C. for 18 h without solvent. HCl (4N in dioxane, 8 mL, 32 mmol) was added and the solution was stirred at room temperature for 2 h then evaporated to dryness. K2CO3 (aqueous, 100 mL, 1M) was added followed by EtOAc (250 mL). The mixture was stirred for 30 minutes followed by removal of the organic layer. The solvent was removed in vacuo, the residue was suspended in Ethanol, and the precipitate collected via vacuum filtration to give 25 mg (12%) product as a light yellow solid. 1H NMR (400 MHz, D6-DMSO) δ ppm 7.85 (m, 1H), 7.79 (d, 1H, J=8 Hz), 7.70 (m, 1H), 7.58 (m, 1H), 7.00 (s, 2H), 6.14 (bs, 2H), 3.42 (m, 2H), 2.65 (t, 2H, J=6 Hz), 2.28 (s, 3H). MS m/z calculated for (M+H)⁺ 411, found 411.

### 5.2.95 5-amino-4-(3,4-dichlorophenyl)-2-[(2-methoxyethyl)amino]thiopheno[2,3-d]pyrimidine-6-carboxamide

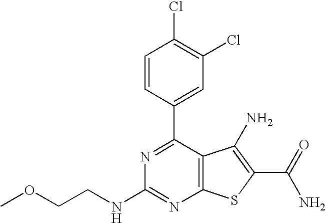

5-amino-4-(3,4-dichlorophenyl)-2-(methylsulfinyl)thiopheno[2,3-d]pyrimidine-6-carboxamide (100 mg, 0.25 mmol), 2-methoxyethylamine (94 mg, 1.25 mmol) and anhydrous THF (1 mL) were placed in a microwave tube and heated to 130° C. for 10 minutes. The volatiles were removed in vacuo, the residue suspended in Ethanol, and the precipitate collected via vacuum filtration to give 31 mg (30%) product as a light yellow solid. ¹H NMR (400 MHz, D6-DMSO) δ ppm 7.86 (s, 1H), 7.79 (d, 1H, J=8 Hz), 7.59 (m, 1H), 7.01 (s, 2H), 6.15 (bs, 2H), 3.49 (m, 4H), 3.26 (s, 2H). MS m/z calculated for (M+H)⁺ 412, found 412.

### 5.2.96 5-amino-4-(3,4-dichlorophenyl)-2-[(2,3-dihydroxypropyl)amino]thiopheno[2,3-d]pyrimidine-6-carboxamide

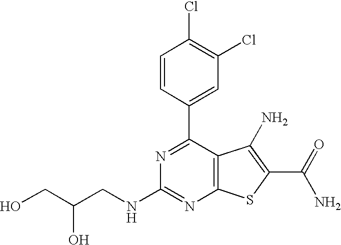

5-amino-4-(3,4-dichlorophenyl)-2-(methylsulfinyl)thiopheno[2,3-d]pyrimidine-6-carboxamide (125 mg, 0.31 mmol) and 3-amino-1,2-propanediol (99.3 mg, 1.09 mmol) were combined and heated at 90° C. for 18 h without solvent. The residue was suspended in Ethanol, and the precipitate collected via vacuum filtration to give 16 mg (12%) product as a light yellow solid. MS m/z calculated for (M+H)⁺ 428, found 428.

### 5.2.97 5-amino-4-(3,4-dichlorophenyl)-2-[(3-hydroxybutyl)amino]thiopheno[2,3-d]pyrimidine-6-carboxamide

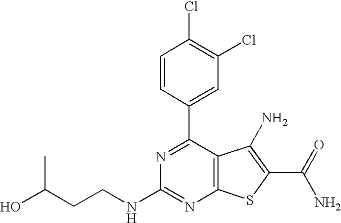

5-amino-4-(3,4-dichlorophenyl)-2-(methylsulfinyl)thiopheno[2,3-d]pyrimidine-6-carboxamide (125 mg, 0.31 mmol) and 4-amino-2-butanol (97 mg, 1.09 mmol) were combined and heated at 90° C. for 18 h without solvent. The crude product was dissolved in DMSO (4 mL) and purified using reverse-phase preparative HPLC (30-80% acetonitrile, 0.1% TFA, 30 minutes). The appropriate fractions were combined and concentrated on a rotary evaporator. The residue was dissolved in ethyl acetate (100 mL), washed with aqueous K₂CO₃, and dried over Na₂SO₄. The solvent was removed in vacuo to give 26 mg (20%) of product as a light yellow solid. ¹H NMR (300 MHz, D6-DMSO) δ ppm 7.85 (s, 1H), 7.79 (d, 1H, J=8.4 Hz), 7.58 (m, 1H), 7.00 (s, 2H), 6.13 (bs, 2H), 4.49 (m, 1H), 3.70 (m, 1H), 3.39 (m, 2H), 1.61 (m, 2H), 1.08 (d, 3H, J=8.4 Hz). MS m/z calculated for (M+H)⁺ 426, found 426.

### 5.2.98 5-amino-4-(3,4-dichlorophenyl)-2-[(3-methoxypropyl)amino]thiopheno[2,3-d]pyrimidine-6-carboxamide

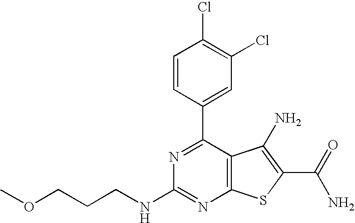

5-amino-4-(3,4-dichlorophenyl)-2-(methylsulfinyl)thiopheno[2,3-d]pyrimidine-6-carboxamide (125 mg, 0.31 mmol) and 3-methoxypropylamine (97 mg, 1.09 mmol) were combined and heated at 90° C. for 18 h without solvent. The crude product was dissolved in DMSO (4 mL) and purified using reverse-phase preparative HPLC (30-80% acetonitrile, 0.1% TFA, 30 minutes). The appropriate fractions were combined and concentrated on a rotary evaporator. The residue was dissolved in ethyl acetate (100 mL), washed with aqueous K₂CO₃, and dried over Na₂SO₄. The solvent was removed in vacuo to give 30 mg (23%) of product as a light yellow solid. ¹H NMR (300 MHz, D6-DMSO) δ ppm 7.86 (s, 1H), 7.79 (d, 1H, J=8.4 Hz), 7.59 (m, 1H), 7.01 (s, 2H), 6.14 (bs, 2H), 3.39 (m, 4H), 3.23 (s, 3H), 1.78 (q, 2H, J=6.9 Hz). MS m/z calculated for (M+H)⁺ 426, found 426.

### 5.2.99 5-amino-4-(3, 4-dichlorophenyl)-2-(butylamino)thiopheno[2,3-d]pyrimidine-6-carboxamide

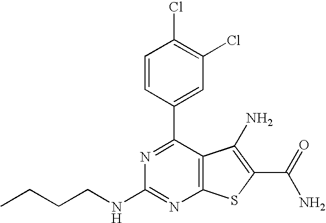

5-amino-4-(3,4-dichlorophenyl)-2-(methylsulfinyl)thiopheno[2,3-d]pyrimidine-6-carboxamide (125 mg, 0.31 mmol) and 1-aminobutane (88 mg, 1.2 mmol) were combined and heated at 90° C. for 18 h without solvent. The residue was suspended in Ethanol, and the precipitate collected via vacuum filtration to give 27 mg (21%) product as a light yellow solid. ¹H NMR (400 MHz, D6-DMSO) δ ppm 7.86 (s, 1H), 7.79 (d, 1H, J=8.4 Hz), 7.58 (m, 1H), 7.01 (s, 2H), 6.13 (bs, 2H), 3.32 (m, 2H), 1.53 (m, 2H), 1.35 (m, 2H), 0.90 (t, 3H, J=8 Hz). MS m/z calculated for (M+H)⁺ 410, found 410.

### 5.2.100 2-[((3S)pyrrolidin-3-yl)amino]-5-amino-4-(3,4-dichlorophenyl)thiopheno[2,3-d]pyrimidine-6-carboxamide

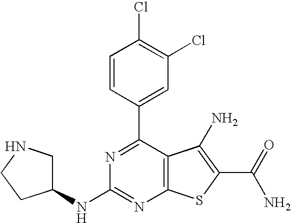

Step a: tert-butyl (3S)-3-{[5-amino-4-(3,4-dichlorophenyl)-6-carbamoylthiopheno[2,3-d]pyrimidin-2-yl]amino}pyrrolidinecarboxylate. 5-amino-4-(3,4-dichlorophenyl)-2-(methylsulfinyl)thiopheno[2,3-d]pyrimidine-6-carboxamide (300 mg, 0.75 mmol) and tert-butyl (3S)-3-aminopyrrolidinecarboxylate (419 mg, 2.25 mmol) were dissolved in THF (10.0 mL) with stirring. The resulting mixture was heated at 70° C. under a reflux condenser for 40 h and then cooled to room temperature. The resulting mixture was concentrated and purified using flash chromatography (0-20% MeOH/CH₂Cl₂) and then dissolved in MeOH and purified using reverse phase preparatory HPLC (20-70% CH₃CN/H₂O) to give 206 mg (52%) of tert-butyl (3S)-3-{[5-amino-4-(3,4-dichlorophenyl)-6-carbamoylthiopheno[2,3-d]pyrimidin-2-yl]amino}pyrrolidinecarboxylate as a light orange solid. MS m/z calculated for (M+2, 4)⁺ 525, 527, found 525, 527.

Step b: 2-[((3S)pyrrolidin-3-yl)amino]-5-amino-4-(3,4-dichlorophenyl)thiopheno[2,3-d]pyrimidine-6-carboxamide. Tert-butyl (3S)-3-{[5-amino-4-(3,4-dichlorophenyl)-6-carbamoylthiopheno[2,3-d]pyrimidin-2-yl]amino}pyrrolidinecarboxylate (206 mg, 0.394 mmol) was dissolved in CH₂Cl₂ (10 mL). Trifluoroacetic acid (1.0 mL) was added dropwise via syringe. The reaction mixture was stirred 4 h at 25° C. The reaction mixture was concentrated and diluted with methanol. Triethylamine (800 uL) was added followed by MP carbonate resin (10 equiv). The resulting mixture was allowed to stir at 25° C. for 18 hours. The resulting mixture was filtered and the MP carbonate resin washed twice with methanol. The filtrate was concentrated on a rotary evaporator to give 27.6 mg (17%) of 2-[((3S)pyrrolidin-3-yl)amino]-5-amino-4-(3,4-dichlorophenyl)thiopheno[2,3-d]pyrimidine-6-carboxamide. ¹H NMR (400 MHz, CD₃OD) δ ppm 1.68-1.76 (m, 1H) 2.06-2.15 (m, 1H) 2.76 (dd, J=12.00, 4.40 Hz, 1H) 2.82-2.88 (m, 1H) 2.96-3.03 (m, 1H) 3.07-3.13 (m, 1H) 4.54 (m, br, 1H) 7.46 (dd, J=2.00, 8.40 Hz, 1H) 7.56 (d, J=8.40, Hz, 1H) 7.72 (d, J=1.60, Hz, 1H). MS m/z calculated for (M+2, 4)⁺ 425, 427 found 425, 427.

### 5.2.101 2-[((3R)pyrrolidin-3-yl)amino]-5-amino-4-(3,4-dichlorophenyl)thiopheno[2,3-d]pyrimidine-6-carboxamide

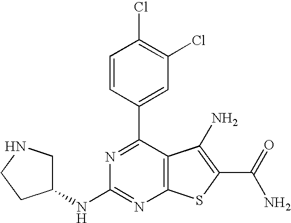

Step a: tert-butyl (3R)-3-{[5-amino-4-(3,4-dichlorophenyl)-6-carbamoylthiopheno[2,3-d]pyrimidin-2-yl]amino}pyrrolidinecarboxylate. 5-amino-4-(3,4-dichlorophenyl)-2-(methylsulfinyl)thiopheno[2,3-d]pyrimidine-6-carboxamide (300 mg, 0.750 mmol) and tert-butyl (3R)-3-aminopyrrolidinecarboxylate (419 mg, 2.25 mmol) were dissolved in THF (10 mL) with stirring. The resulting mixture was heated at 70° C. under a reflux condenser for 18 h and then cooled to room temperature. The resulting mixture was concentrated and purified using flash chromatography (0-20% MeOH/CH₂Cl₂) to give 206 mg (52%) of tert-butyl (3R)-3-{[5-amino-4-(3,4-dichlorophenyl)-6-carbamoylthiopheno[2,3-d]pyrimidin-2-yl]amino}pyrrolidinecarboxylate as a light orange solid. MS m/z calculated for (M+2, 4)⁺ 525, 527 found 525, 527.

Step b: 2-[((3R)pyrrolidin-3-yl)amino]-5-amino-4-(3,4-dichlorophenyl)thiopheno[2,3-d]pyrimidine-6-carboxamide. Tert-butyl (3R)-3-{[5-amino-4-(3,4-dichlorophenyl)-6-carbamoylthiopheno[2,3-d]pyrimidin-2-yl]amino}pyrrolidinecarboxylate (281 mg, 0.537 mmol) was dissolved in CH₂Cl₂ (10 mL). Trifluoroacetic acid (1.0 mL) was added dropwise via syringe. The reaction mixture was stirred 18 h at 25° C. The resulting crude material was dissolved in MeOH and purified using reverse phase preparatory HPLC (20-70% CH₃CN/H₂O) to give 126 mg of 2-[((3R)pyrrolidin-3-yl)amino]-5-amino-4-(3,4-dichlorophenyl)thiopheno[2,3-d]pyrimidine-6-carboxamide as a light orange solid. The clean fractions were concentrated and diluted with methanol. Triethylamine (800 uL) was added followed by MP carbonate resin (10 equiv). The resulting mixture was allowed to stir at 25° C. for 18 hours. The resulting mixture was filtered and the MP carbonate resin washed twice with methanol. The filtrate was concentrated on a rotary evaporator to give 72.0 mg (32%) of 2-[((3R)pyrrolidin-3-yl)amino]-5-amino-4-(3,4-dichlorophenyl)thiopheno[2,3-d]pyrimidine-6-carboxamide. ¹H NMR (400 MHz, CD₃OD)₆ ppm 1.77-1.82 (m, 1H) 2.16-2.21 (m, 1H) 2.79 (dd, J=11.60, 4.40 Hz, 1H) 2.89-2.95 (m, 1H) 3.03-3.09 (m, 1H) 3.14-3.19 (m, 1H) 4.54 (m, br, 1H) 7.54 (dd, J=2.00, 8.40 Hz, 1H) 7.68 (d, J=8.00, Hz, 1H) 7.81 (d, J=1.60, Hz, 1H). MS m/z calculated for (M+2, 4)⁺ 425, 427 found 425, 427.

### 5.2.102 5-amino-4-(3,4-dichlorophenyl)-2-(4-piperidylamino)thiopheno[2,3-d]pyrimidine-6-carboxamide

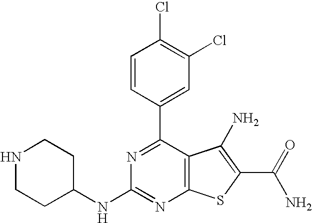

Step a: tert-butyl 4-{[5-amino-4-(3,4-dichlorophenyl)-6-carbamoylthiopheno[2,3-d]pyrimidin-2-yl]amino}piperidinecarboxylate. 5-amino-4-(3,4-dichlorophenyl)-2-(methylsulfinyl)thiopheno[2,3-d]pyrimidine-6-carboxamide (300 mg, 0.750 mmol) and tert-butyl 4-aminopiperidinecarboxylate (457 mg, 2.25 mmol) were dissolved in THF (10 mL) with stirring. The resulting mixture was heated at 70° C. under a reflux condenser for 18 h and then cooled to room temperature. The resulting mixture was concentrated and purified using flash chromatography (0-20% MeOH/CH₂Cl₂) to give 179 mg (44%) of tert-butyl 4-{[5-amino-4-(3,4-dichlorophenyl)-6-carbamoylthiopheno[2,3-d]pyrimidin-2-yl]amino}piperidinecarboxylate. MS m/z calculated for (M+1)⁺ 538, found 538.

Step b: 5-amino-4-(3,4-dichlorophenyl)-2-(4-piperidylamino)thiopheno[2,3-d]pyrimidine-6-carboxamide. Tert-butyl 4-{[5-amino-4-(3,4-dichlorophenyl)-6-carbamoylthiopheno[2,3-d]pyrimidin-2-yl]amino}piperidinecarboxylate (179 mg, 0.333 mmol) was dissolved in CH₂Cl₂ (10 mL). Trifluoroacetic acid (1.0 mL) was added dropwise via syringe. The reaction mixture was stirred 18 h at 25° C. The resulting crude material was dissolved in MeOH and purified using reverse phase preparatory HPLC (20-70% CH₃CN/H₂O) to give 186 mg of a yellow solid which was diluted with methanol. Triethylamine (800 uL) was added followed by MP carbonate resin (10 equiv). The resulting mixture was allowed to stir at 25° C. for 18 hours. The resulting mixture was filtered and the MP carbonate resin washed twice with methanol. The filtrate was concentrated on a rotary evaporator to give 45.6 mg (31%) of 5-amino-4-(3,4-dichlorophenyl)-2-(4-piperidylamino)thiopheno[2,3-d]pyrimidine-6-carboxamide. ¹H NMR (400 MHz, CD₃OD) δ ppm 1.47-1.54 (m, 2H) 2.01-2.03 (m, 2H) 2.67 (t, J=11.60, Hz, 2H) 3.06-3.09 (m, 2H) 3.99 (m, br, 1H) 7.53 (dd, J=2.00, 8.00 Hz, 1H) 7.68 (d, J=8.00, Hz, 1H) 7.80 (d, J=2.00, Hz, 1H). MS m/z calculated for (M+2, 4)⁺ 437.32, 439.32 found 437, 439.

### 5.2.103 5-amino-2-(azetidin-3-ylamino)-4-(3,4-dichlorophenyl)thiopheno[2,3-d]pyrimidine-6-carboxamide

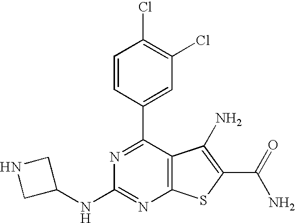

Step a: tert-butyl 3-{[5-amino-4-(3,4-dichlorophenyl)-6-carbamoylthiopheno[2,3-d]pyrimidin-2-yl]amino}azetidinecarboxylate. 5-amino-4-(3,4-dichlorophenyl)-2-(methylsulfinyl)thiopheno[2,3-d]pyrimidine-6-carboxamide (300 mg, 0.750 mmol) and tert-butyl 3-aminoazetidinecarboxylate (387.5 mg, 2.25 mmol) were dissolved in THF (10 mL) with stirring. The resulting mixture was heated at 70° C. under a reflux condenser for 18 h and then cooled to room temperature. The resulting mixture was concentrated and purified using flash chromatography (0-20% MeOH/CH₂Cl₂) to give 211 mg (55%) of tert-butyl 3-{[5-amino-4-(3,4-dichlorophenyl)-6-carbamoylthiopheno[2,3-d]pyrimidin-2-yl]amino}azetidinecarboxylate. MS m/z calculated for (M+1)⁺ 510, found 510.

Step b: 5-amino-2-(azetidin-3-ylamino)-4-(3,4-dichlorophenyl)thiopheno[2,3-d]pyrimidine-6-carboxamide. Tert-butyl 3-{[5-amino-4-(3,4-dichlorophenyl)-6-carbamoylthiopheno[2,3-d]pyrimidin-2-yl]amino}azetidinecarboxylate (211 mg, 0.414 mmol) was dissolved in CH₂Cl₂ (10 mL). Trifluoroacetic acid (1.0 mL) was added dropwise via syringe. The reaction mixture was stirred 18 hours at 25° C. The resulting crude material was dissolved in MeOH and purified using reverse phase preparatory HPLC (20-70% CH₃CN/H₂O) to give 185 mg of a yellow solid which was diluted with methanol. Triethylamine (800 uL) was added followed by MP carbonate resin (10 equiv). The resulting mixture was allowed to stir at 25° C. for 18 hours. The resulting mixture was filtered and the MP carbonate resin washed twice with methanol. The filtrate was concentrated on a rotary evaporator to give 63.6 mg (38%) of 5-amino-2-(azetidin-3-ylamino)-4-(3,4-dichlorophenyl)thiopheno[2,3-d]pyrimidine-6-carboxamide. ¹H NMR (400 MHz, CD₃OD) δ ppm 3.14-3.17 (m, 1H) 3.64-3.68 (m, 1H) 3.64-3.68 (m, 1H) 7.57 (dd, J=2.00, 8.40 Hz, 1H) 7.68 (d, J=8.40, Hz, 1H) 7.80 (d, J=2.00, Hz, 1H). MS m/z calculated for (M+2, 4)⁺ 426.32, 428.32 found 426, 428.

### 5.2.104 2-[((3S)oxolan-3-yl)amino]-5-amino-4-(3,4-dichlorophenyl)thiopheno[2,3-d]pyrimidine-6-carboxamide

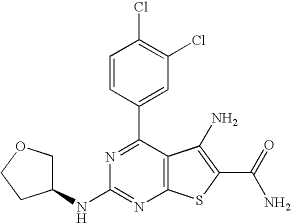

5-amino-4-(3,4-dichlorophenyl)-2-(methylsulfinyl)thiopheno[2,3-d]pyrimidine-6-carboxamide (300 mg, 0.750 mmol) and (3S)oxolane-3-ylamine (280 mg, 2.25 mmol) were dissolved in THF (10 mL) with stirring. The resulting mixture was heated at 70° C. under a reflux condenser for 18 hours and then cooled to room temperature. The resulting mixture was concentrated, dissolved in MeOH, and purified using reverse phase preparatory HPLC (20-70% CH₃CN/H₂O) to give 146 mg of a yellow solid which was diluted with methanol. Triethylamine (800 uL) was added followed by MP carbonate resin (10 equiv). The resulting mixture was allowed to stir at 25° C. for 18 hours. The resulting mixture was filtered and the MP carbonate resin washed twice with methanol. The filtrate was concentrated on a rotary evaporator to give 34.0 mg (43%) of 2-[((3S)oxolan-3-yl)amino]-5-amino-4-(3,4-dichlorophenyl)thiopheno[2,3-d]pyrimidine-6-carboxamide. ¹H NMR (400 MHz, DMSO d6) δ ppm 1.87-1.95 (m, 1H) 2.14-2.19 (m, 1H) 3.56 (dd, J=4.00, 8.80 Hz, 1H) 3.71-3.75 (m, 1H) 3.80-3.91 (m, 2H) 4.46 (m, br, 1H) 6.16 (s, br, 2H) 6.16 (s, 1H) 7.56 (s, br, 1H) 7.78 (d, J=8.40, Hz, 2H) 7.87 (d, J=1.60, Hz, 1H) 8.11 (s, br, 1H). MS m/z calculated for (M+2, 4)⁺ 426, 428 found 426, 428.

### 5.2.105 2-[((3R)oxolan-3-yl)amino]-5-amino-4-(3,4-dichlorophenyl)thiopheno[2,3-d]pyrimidine-6-carboxamide

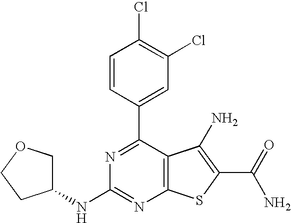

5-amino-4-(3,4-dichlorophenyl)-2-(methylsulfinyl)thiopheno[2,3-d]pyrimidine-6-carboxamide (300 mg, 0.750 mmol) and (3R)oxolane-3-ylamine (280.1 mg, 2.25 mmol) were dissolved in DMF (4.0 mL) with stirring. The resulting mixture was heated at 90° C. under a reflux condenser for 18 hours and then cooled to room temperature. The resulting mixture was concentrated, dissolved in MeOH, and purified using reverse phase preparatory HPLC (20-70% CH₃CN/H₂O) to give 157 mg of a yellow solid which was diluted with methanol. Triethylamine (800 uL) was added followed by MP carbonate resin (10 equiv). The resulting mixture was allowed to stir at 25° C. for 18 hours. The resulting mixture was filtered and the MP carbonate resin washed twice with methanol. The filtrate was concentrated on a rotary evaporator to give 63.3 mg (20%) of 2-[((3R)oxolan-3-yl)amino]-5-amino-4-(3,4-dichlorophenyl)thiopheno[2,3-d]pyrimidine-6-carboxamide. ¹H NMR (400 MHz, DMSO d6) δ ppm 1.88-1.95 (m, 1H) 2.14-2.19 (m, 1H) 3.56 (dd, J=4.40, 8.80 Hz, 1H) 3.70-3.75 (m, 1H) 3.81-3.91 (m, 2H) 4.45 (m, br, 1H) 6.16 (s, br, 2H) 7.04 (s, 2H) 7.59 (s, br, 1H) 7.78 (d, J=8.40, Hz, 1H) 7.87 (s, 1H) 8.10 (s, br, 1H). MS m/z calculated for (M+2, 4)⁺ 426, 428 found 426, 428.

### 5.2.106 5-amino-2-(cyclopropylamino)-4-(2-fluoro-5-methoxyphenyl)thieno[2,3-d]pyrimidine-6-carboxamide

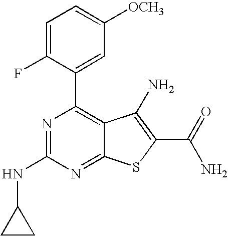

Step a: 4-(2-fluoro-5-methoxyphenyl)-6-hydroxy-2-(methylthio)pyrimidine-5-carbonitrile. 2-fluoro-5-methoxybenzaldehyde (2.00 g, 13 mmol), 1-cyclopropylguanidine hydrochloride (1.76 g, 13 mmol), ethyl 2-cyanoacetate (1.62 g, 14.3 mmol), and potassium carbonate (1.97 g, 14.3 mmol) were mixed in 40 mL dry EtOH under N₂ and heated at 65° C. for two hours. The solvent was removed and the residue partitioned between water and ethyl acetate (75 mL each). The organic layer was dried and concentrated to yield the desired product as a yellow solid (3.5 g, 89.7% yield). MS m/z calculated for (M+H)⁺ 301, found 301.

Step b: 4-chloro-6-(2-fluoro-5-methoxyphenyl)-2-(methylthio)pyrimidine-5-carbonitrile. 4-(2-fluoro-5-methoxyphenyl)-6-hydroxy-2-(methylthio)pyrimidine-5-carbonitrile (3.5 g, 11.7 mmol) was dissolved in 40 mL dry 1,4-dioxane under N₂ and phosphorus oxychloride (8 mL, 86 mmol) and a few drops of dry DMF were added. The reaction mixture was heated to reflux for two hours and quenched by adding ice/water. The resulting mixture was extracted with EtOAc to yield the desired product (3.2 g, 86% yield). MS m/z calculated for (M+H)⁺ 319, found 319.

Step c: 2-(5-cyano-6-(2-fluoro-5-methoxyphenyl)-2-(methylthio)pyrimidin-4-ylthio)acetamide. 4-chloro-6-(2-fluoro-5-methoxyphenyl)-2-(methylthio)pyrimidine-5-carbonitrile (3.2 g, 10 mmol), 2-mercaptoacetamide (1.0 g, 111 mmol) and sodium carbonate (1.16 g, 11 mmol) were suspended in 60 mL dry EtOH under N₂ and heated at 60° C. for two hours. The solvent was removed and the residue partitioned between water and ethyl acetate (80 mL each). The organic layer was separated, dried over MgSO₄, and concentrated. Purification using flash chromatography (silica gel, 25-100% EtOAc/hexane) gave the desired product (1.5 g, 40% yield). MS m/z calculated for (M+H)⁺ 374, found 374.

Step d: 5-amino-2-(cyclopropylamino)-4-(2-fluoro-5-methoxyphenyl)thieno[2,3-d]pyrimidine-6-carboxamide. 2-(5-cyano-6-(2-fluoro-5-methoxyphenyl)-2-(methylthio)pyrimidin-4-ylthio)acetamide (1.5 g, 4 mmol) was placed in anhydrous ethanol (40 mL) and sodium metal was added in small portions (0.55 g, 24 mmol) at room temperature. After addition was complete the mixture was heated to reflux for two hours, cooled, and the solvent removed. The solid obtained was treated with water and acidified to pH 6 with dilute acetic acid then extracted with ethyl acetate (100 mL). Upon concentration and sonication with hexane:ethyl acetate (3:1) a solid was isolated by filtration to yield the desired product (1.0 g, 67% yield). ¹H NMR (400 MHz, DMSO-d6) δ ppm 8.03 (bs, 1H) 7.32 (t, 1H) 7.15 (m, 1H) 7.09 (m, 1H) 5.87 (bs, 2H) 3.77 (s, 3H) 2.82 (m, 1H) 0.74 (bs, 2H) 0.52 (bs, 2H). MS m/z calculated for (M+H)⁺ 374, found 374.

### 5.2.107 5-amino-2-(cyclopropylamino)-4-(2-fluoro-5-hydroxyphenyl)thieno[2,3-d]pyrimidine-6-carboxamide

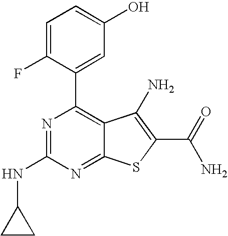

5-amino-2-(cyclopropylamino)-4-(2-fluoro-5-methoxyphenyl)thieno[2,3-d]pyrimidine-6-carboxamide (0.373 g, 1 mmol) was dissolved in 20 mL of CH₂Cl₂ and BBr₃ (1M in CH₂Cl₂, 3 mL) was added. The mixture was stirred at room temperature overnight. The reaction was quenched with ice water. Na₂CO₃ was added to make the pH 6. The organic layer was concentrated and purified using reverse phase preparative HPLC (Method 10-100%, 30 minutes, 20 mL/min) to yield the desired product (0.17 g, 47% yield). ¹H NMR (300 MHz, DMSO-d6) δ ppm 9.86 (s, 1H) 8.05 (bs, 1H) 7.28 (t, 1H) 7.09 (bs, 2H) 7.03 (m, 1H) 6.88 (m, 1H) 5.98 (bs, 2H) 2.88 (m 1H) 0.78 (m, 2H) 0.59 (m, 2H). MS m/z calculated for (M+H)⁺ 360, found 360.

### 5.2.108 5-amino-2-(cyclopropylamino)-4-(2-fluoro-3-hydroxyphenyl)thieno[2,3-d]pyrimidine-6-carboxamide

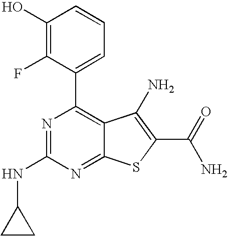

5-amino-2-(cyclopropylamino)-4-(2-fluoro-3-methoxyphenyl)thieno[2,3-d]pyrimidine-6-carboxamide (0.4 g, 1.1 mmol) was placed in 20 mL of dry CH₂Cl₂ and BBr₃ (1M in CH₂Cl₂, 3.2 mL) was added. The mixture was stirred at room temperature overnight and quenched with water. The CH₂Cl₂ was removed and the residue made basic with NH₄OH, extracted with ethyl acetate, and purified using reverse phase preparative HPLC (Method 10-100%, 30 minutes, 20 mL/min) to yield the desired product (0.087 g, 23% yield). ¹H NMR (400 MHz, DMSO-D6) δ ppm 0.48-0.55 (m, 2H) 0.66-0.75 (m, J=3.12 Hz, 2H) 2.75-2.84 (m, 1H) 5.84 (s, 2H) 6.82-6.91 (m, 1H) 7.01 (s, 2H) 7.08-7.19 (m, 2H) 10.23 (s, 1H). MS m/z calculated for (M+H)⁺ 360, found 360.

### 5.2.109 5-amino-2-(cyclopropylamino)-4-[3-methoxy-4-(trifluoromethyl)phenyl]thiopheno[2,3-d]pyrimidine-6-carboxamide

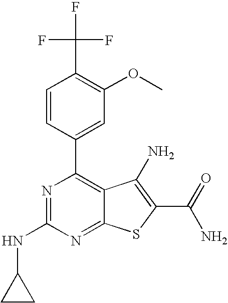

Step a: 3-Methoxy-4-(trifluoromethyl)benzaldehyde. 3-Fluoro-4(trifluoromethyl)benzoic acid (8 g, 38.5 mmol) was placed in 70 mL of DMSO and treated with NaOMe (5.2 g, 96 mmol) in 20 mL of dry MeOH. The mixture was heated to reflux for 3 hours then poured into 300 mL of ice/water and acidified with dilute HCl. The precipitate obtained was filtered and dried under high vacuum. The resulting solids were placed in 60 mL of dry THF under N₂ and cooled to 0° C. LAH (1 M in THF, 40 mL) was added via syringe. The ice bath was removed and the reaction mixture stirred at room temperature for 2 hours. The reaction was recooled to 0° C. and the reaction quenched by addition of 4 mL of 2 N NaOH under an N₂ atmosphere. The precipitate was filtered and washed with more THF. The filtrate was concentrated and dried under high vacuum. The residue was dissolved in 100 mL of CH₂Cl₂ and Dess-martin reagent (0.3 M in CH₂Cl₂, 75 mL) was added via syringe. The solution was stirred for 30 minutes and then treated with ethyl ether (50 mL) and an aqueous NaOH solution (8.8 g in 100 mL of water) for 10 minutes. The organic layer was separated, washed with water and dried to yield the desired product as a yellow oil (3 g, 38% yield). ¹H NMR (400 MHz, CDCl₃) δ ppm 3.99 (s, 3H) 7.51 (m, 2H) 7.77 (d, 1H) 10.05 (s, 1H). MS m/z calculated for (M+H)⁺ 205, found 205.

Step b: 2-(cyclopropylamino)-4-hydroxy-6-(3-methoxy-4-(trifluoromethyl)phenyl)pyrimidine-5-carbonitrile. 3-Methoxy-4-(trifluoromethyl)benzaldehyde (3 g, 14.7 mmol), 1-cyclopropylguanidine hydrochloride (1.99 g, 14.7 mmol), ethyl 2-cyanoacetate (1.66 g, 14.7 mmol), and potassium carbonate (2.23 g, 16.1 mmol) were mixed in 40 mL dry EtOH under N₂ and heated at 65° C. for two hours. The solvent was removed and the residue treated with water and ethyl acetate (75 mL each). The organic layer was dried and concentrated under high vacuum to yield the desired product as a yellow solid (4.8 g. 93.3% yield). MS m/z calculated for (M+H)⁺ 351, found 351.

Step c: 4-chloro-2-(cyclopropylamino)-6-(3-methoxy-4-(trifluoromethyl)phenyl)pyrimidine-5-carbonitrile. 2-(cyclopropylamino)-4-hydroxy-6-(3-methoxy-4-(trifluoromethyl)phenyl)pyrimidine-5-carbonitrile (4.8 g, 13.7 mmol) was dissolved in 40 mL dry 1,4-dioxane under N₂ and phosphorus oxychloride (13 mL, 137 mmol) and a few drops of dry DMF were added. The reaction mixture was heated to reflux for two hours and quenched by adding ice/water. The reaction mixture was extracted with EtOAc to yield the desired product (3.62 g, 72% yield). MS 7/z calculated for (M+H)⁺ 369, found 369.

Step d: 2-(5-cyano-2-(cyclopropylamino)-6-(3-methoxy-4-(trifluoromethyl)phenyl)pyrimidin-4-ylthio)acetamide. 4-chloro-2-(cyclopropylamino)-6-(3-methoxy-4-(trifluoromethyl)phenyl)pyrimidine-5-carbonitrile (3.62 g, 9.8 mmol), 2-mercaptoacetamide (0.89 g, 9.8 mmol), and sodium carbonate (1.04 g, 9.8 mmol) were suspended in 60 mL dry EtOH under N₂ and stirred at room temperature for 48 hours. The solvent was removed and the residue treated with water and ethyl acetate (80 mL each). The organic layer was separated, dried (anhydrous MgSO₄) and concentrated. The crude product was used without further purification for the next step. MS m/z calculated for (M+H)⁺ 424, found 424.

Step e: 5-amino-2-(cyclopropylamino)-4-(3-methoxy-4-(trifluoromethyl)phenyl)thieno[2,3-d]pyrimidine-6-carboxamide. 2-(5-cyano-2-(cyclopropylamino)-6-(3-methoxy-4-(trifluoromethyl)phenyl)pyrimidin-4-ylthio)acetamide (4.1 g, 9.8 mmol), was placed in anhydrous ethanol (40 mL) and sodium metal (2.2 g, 98 mmol) was added in small portions at room temperature. After addition was complete the mixture was heated to reflux for two hours, cooled and the solvent removed. The solid obtained was treated with water and acidified to pH 6 with dilute acetic acid and extracted with ethyl acetate (100 mL). Purification using flash chromatography (silica gel, hexane:ethyl acetate 2:3) yielded the desired product (0.8 g, 19.3% yield). ¹H NMR (400 MHz, DMSO-D6) δ ppm 0.47-0.58 (m, 2H) 0.65-0.76 (m, 2H) 2.74-2.91 (m, 1H) 3.86-4.00 (m, 3H) 6.06 (s, 2H) 6.94-7.07 (m, 2H) 7.21-7.34 (m, 1H) 7.42-7.50 (m, 1H) 7.76 (d, 1H) 7.94-8.05 (m, 1H). MS m/z calculated for (M+H)⁺ 424, found 424.

### 5.2.110 5-amino-2-(cyclopropylamino)-4-[3-hydroxy-4-(trifluoromethyl)phenyl]thiopheno[2,3-d]pyrimidine-6-carboxamide

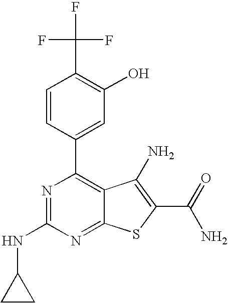

5-amino-2-(cyclopropylamino)-4-(3-methoxy-4-(trifluoromethyl)phenyl)thieno[2,3-d]pyrimidine-6-carboxamide (0.4 g, 0.94 mmol) was placed in 20 mL of dry CH₂Cl₂ and BBr₃ (1M in CH₂Cl₂, 6 mL) was added. The mixture was stirred at room temperature overnight and quenched with water. The CH₂Cl₂ was removed and the residue was treated with NH₄OH to pH 9, extracted into EtOAc and purified by Prep HPLC to yield the desired product (0.075 g, 19% yield). ¹H NMR (400 MHz, DMSO-D6) δ ppm 0.47-0.56 (m, J=4.10, 4.10 Hz, 2H) 0.66-0.78 (m, 2H) 2.76-2.88 (m, 1H) 6.07 (s, 2H) 7.02 (s, 2H) 7.08-7.24 (m, 2H) 7.67 (d, J=8.20 Hz, 1H) 8.01 (s, 1H) 10.97 (s, 1H). MS m/z calculated for (M+H)⁺ 410, found 410.

### 5.2.111 5-amino-2-(cyclopropylamino)-4-(4-(trifluoromethyl)phenyl)thieno[23-d]pyrimidine-6-carboxamide

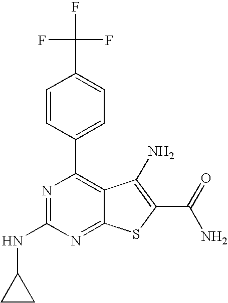

Step a: 2-(cyclopropylamino)-4-hydroxy-6-(4-(trifluoromethyl)phenyl)pyrimidine-5-carbonitrile. 4-trifluoromethylbenzaldehyde (2.6 g, 15 mmol), 1-cyclopropylguanidine hydrochloride (2.03 g, 15 mmol), ethyl 2-cyanoacetate (1.69 g, 15 mmol), and potassium carbonate (2.28 g, 16.5 mmol) were mixed in 60 mL dry EtOH under N₂ and heated at 50° C. overnight. The reaction was then concentrated and treated with water and EtOAc (100 mL each). The organic layer was dried and concentrated to yield an oil (3 g, 62% yield). MS m/z calculated for (M+H)⁺ 321, found 321.

Step b: 4-chloro-2-(cyclopropylamino)-6-(4-(trifluoromethyl)phenyl)pyrimidine-5-carbonitrile. 2-(cyclopropylamino)-4-hydroxy-6-(4-(trifluoromethyl)phenyl)pyrimidine-5-carbonitrile (3 g, 9.3 mmol) was dissolved in 60 mL dry 1,4-dioxane under N₂ and phosphorus oxychloride (8 mL, 84 mmol) and a few drops of dry DMF were added. The reaction mixture was heated at 70° C. for 3 hours and quenched by adding ice/water. The reaction mixture was treated with dilute NaOH to pH 6 and extracted with EtOAc to yield the desired product (1.78 g, 56% yield). MS m/z calculated for (M+H)⁺ 339, found 339.

Step c: 2-(5-cyano-2-(cyclopropylamino)-6-(4-(trifluoromethyl)phenyl)pyrimidin-4-ylthio)acetamide. 4-chloro-2-(cyclopropylamino)-6-(4-(trifluoromethyl)phenyl)pyrimidine-5-carbonitrile (1.78 g, 5.2 mmol), 2-mercaptoacetamide (0.52 g, 5.8 mmol), and sodium carbonate (0.61 g, 5.8 mmol) were suspended in 60 mL dry EtOH under N₂ and stirred at room temperature for 48 hours. The solvent was removed and the residue treated with EtOAc and water (100 mL each). The organic layer was dried and concentrated to yield crude desired product (2 g, 95% yield). MS m/z calculated for (M+H)⁺ 394, found 394.

Step d: 5-amino-2-(cyclopropylamino)-4-(4-(trifluoromethyl)phenyl)thieno[2,3-d]pyrimidine-6-carboxamide. 2-(5-cyano-2-(cyclopropylamino)-6-(4-(trifluoromethyl)phenyl)pyrimidin-4-ylthio)acetamide (2 g, 5 mmol), was placed in anhydrous ethanol (40 mL) and sodium metal was added in small portions (1 g, 43 mmol) at room temperature. After addition was complete the mixture was heated to reflux for two hours, cooled, and the solvent removed. The solid obtained was treated with water and acidified to pH 6 with dilute acetic acid and extracted with ethyl acetate (100 mL). Purification using flash chromatography (silica gel, hexane:ethyl acetate 2:3) and prep HPLC yielded the desired product (0.1 g, 4.6% yield). ¹H NMR (400 MHz, DMSO-D6) δ ppm 0.46-0.57 (m, 2H) 0.66-0.79 (m, 2H) 2.75-2.88 (m, 1H) 6.00 (s, 2H) 7.02 (s, 2H) 7.81 (s, 2H) 7.87-7.94 (m, 2H) 7.99 (s, 1H). MS m/z calculated for (M+H)⁺ 394, found 394.

### 5.2.112 5-Amino-4-(2-chloropyridin-4-yl)-2-(cyclopropylamino)thieno[2,3-d]pyrimidine-6-carboxamide

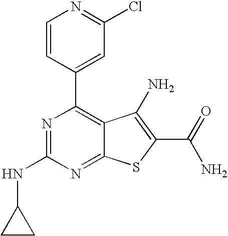

Step a: 2-(Benzyloxy)isonicotinaldehyde. The title compound was prepared from 2-chloroisonicotinic acid by standard procedures. ¹H NMR (300 MHz, CDCl₃) δ ppm 5.43 (s, 2H) 7.5-7.2 (m, 7H) 8.4 (d, 1H) 10 (s, 1H).

Step b: 4-(2-(Benzyloxy)pyridin-4-yl)-2-(cyclopropylamino)-6-hydroxypyrimidine-5-carbonitrile. 2-(Benzyloxy)isonicotinaldehyde (1.38 g, 6.5 mmol), 1-cyclopropylguanidine hydrochloride (0.88 g, 6.5 mmol), ethyl 2-cyanoacetate (0.73 g, 6.5 mmol), and potassium carbonate (0.99 g, 7.15 mmol) were mixed in 30 mL dry EtOH under N₂ and heated at 70° C. for 2 hrs. The solvent was removed and the residue treated with water and EtOAc. The organic layer was concentrated and dried under high vacuum to yield the desired product (1.5 g, 64% yield). MS m/z calculated for (M+H)⁺ 360, found 360.

Step c: 4-Chloro-6-(2-chloropyridin-4-yl)-2-(cyclopropylamino)pyrimidine-5-carbonitrile. 4-(2-(Benzyloxy)pyridin-4-yl)-2-(cyclopropylamino)-6-hydroxypyrimidine-5-carbonitrile (1.5 g, 4.18 mmol) was treated with POCl₃ (1.92 g, 12.5 mmol) in 1,4-dioxane at 70° C. for 2 hours. Removal of the solvent and extraction with EtOAc followed using flash chromatography (silica, hexane:EtOAc (2:1 to 1:1) yielded the desired product (0.2 g, 13% yield). ¹H NMR (300 MHz, CDCl3) δ ppm 0.66 (m, 2H) 0.96 (m, 2H) 2.98 (m, 1H) 6.0 (d, 1H) 7.7-7.9 (m, 2H) 8.6 (m, 1H). MS m/z calculated for (M+H)⁺ 306/308, found 306/308 (di-chloro pattern).

Step d: 5-Amino-4-(2-chloropyridin-4-yl)-2-(cyclopropylamino)thieno[2,3-d]pyrimidine-6-carboxamide. 4-Chloro-6-(2-chloropyridin-4-yl)-2-(cyclopropylamino)pyrimidine-5-carbonitrile (0.2 g, 0.65 mmol), 2-mercaptoacetamide (0.06 g, 0.65 mmol), and sodium carbonate (0.07 g, 0.65 mmol) were suspended in 20 mL dry EtOH under N₂ and stirred at room temperature for 48 hours. The solvent was removed and the residue was treated with water and the resulting solid was isolated by filtration. The solid was placed in 1:1 water:ethanol (30 mL each) and 1 N NaOH (2 mmol) was added. The resulting mixture was heated to 60° C. for 30 minutes and treated with 60 mL of EtOAc. The organic layer was separated and purified by Prep HPLC to yield the desired product (0.088 g, 52% yield). ¹H NMR (300 MHz, DMSO-D6) δ ppm 0.47-0.57 (m, 2H) 0.66-0.77 (m, 2H) 2.76-2.89 (m, 1H) 6.18 (s, 2H) 7.07 (s, 2H) 7.57-7.65 (m, 1H) 7.72 (s, 1H) 8.09 (s, 1H) 8.57 (d, J=4.94 Hz, 1H). MS m/z calculated for (M+H)⁺ 361, found 361.

### 5.2.113 5-Amino-2(cyclopropylamino)-4-(2-hydroxypiridin-4-yl)thieno[2,3-d]pyrimidine-6-carboxamide

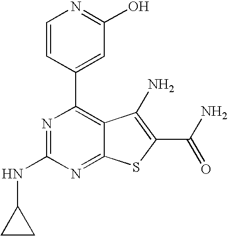

Step a: 4-(2-Benzyloxy)pyridin-4-yl)-6-chloro-2-(cyclopropylamino)pyrimidine-5-carbonitrile. 4-(2-(Benzyloxy)pyridin-4-yl)-2-(cyclopropylamino)-6-hydroxypyrimidine-5-carbonitrile (0.13 g, 0.36 mmol) and POCl₃ (0.035 mL) were placed in 1,4-dioxane (10 mL) and a catalytic amount of DMF added. The mixture was heated at 80° C. for 2 hours. The mixture was cooled to room temperature. The solvent was removed and treated with water, extracted into EtOAc to yield crude product (0.08 g, 59% yield). MS m/z calculated for (M+H)⁺ 378, found 378.

Step b: 5-Amino-2(cyclopropylamino)-4-(2-hydroxypiridin-4-yl)thieno[2,3-d]pyrimidine-6-carboxamide. 4-(2-Benzyloxy)pyridin-4-yl)-6-chloro-2-(cyclopropylamino)pyrimidine-5-carbonitrile (0.08 g, 0.2 mmol), 2-mercaptoacetamide (0.02 g, 0.2 mmol), and sodium carbonate (0.02 g, 0.2 mmol) were heated to reflux in 10 mL of EtOH for 2 hours. To this mixture was then added Na metal (0.015 g, 0.6 mmol) and refluxing continued for 1 hour. The flask was cooled to room temperature and the solvent removed. Treatment with water and EtOAc (10 mL each) and separation of the organic layer yielded a solid that was dissolved in 10 mL of EtOH. A few drops of HOAc was added and the mixture hydrogenated in the presence of 10% Pd/C (1 atm, overnight). The reaction was purified by Prep. HPLC to yield the desired product (0.004 g, 6.6% yield). ¹H NMR (400 MHz, DMSO-D6) δ ppm 0.39-0.55 (m, 2H) 0.62-0.88 (m, 2H) 2.72-2.94 (m, 1H) 6.18-6.37 (m, 3H) 6.34-6.50 (m, 2H) 7.01 (s, 2H) 7.42-7.60 (m, 1H) 7.91-8.14 (m, 1H). MS m/z calculated for (M+H)⁺ 343, found 343.

### 5.2.114 5-Amino-4-(3,4-dichlorophenyl)-2-isopropylthieno[2,3-d]pyrimidine-6-carboxamide

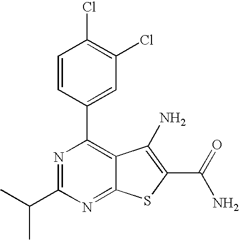

Step a: 4-(3,4-dichlorophenyl)-6-hydroxy-2-isopropylpyrimidine-5-carbonitrile. 3,4-dichlorobenzaldehyde (4.2 g, 24 mmol), isopropylcarbamidine hydrochloride (2.94 g, 24 mmol), ethylcyanoacetate (2.7 g, 24 mmol), and K₂CO₃ (3.65 g, 26.4 mmol) were mixed in 100 mL dry EtOH under N₂ and heated at 50° C. for 2 hours. The solvent was removed and the residue treated with water and EtOAc. The organic layer was concentrated and dried under high vacuum to yield the desired product (4.1 g, 55% yield). MS m/z calculated for (M+H)⁺ 308/310, found 308/310 (di-chloro pattern).

Step b: 4-chloro-6-(3,4-dichlorophenyl)-2-isopropylpyrimidine-5-carbonitrile. 4-(3,4-dichlorophenyl)-6-hydroxy-2-isopropylpyrimidine-5-carbonitrile (4.1 g, 13.3 mmol) was dissolved in 100 mL of 1,4-dioxane and few drops of DMF added. POCl₃ (6.5 mL, 66.5 mmol) was added and the solution heated to 70° C. for 2 hours then allowed to stir at room temperature overnight. The solvent was removed and ice was added to quench excess POCl₃. The resulting mixture was extracted with EtOAc, concentrated to dryness, and dried under high vacuum to yield the desired product (3.75 g, 86% yield). MS m/z calculated for (M+H)⁺ 325/327, found 325/327 (di-chloro pattern).

Step c: 2-(5-cyano-6-(3,4-dichlorophenyl)-2-isopropylpyrimidin-4-ylthio)acetamide. 4-chloro-6-(3,4-dichlorophenyl)-2-isopropylpyrimidine-5-carbonitrile (3.75 g, 11.5 mmol), 2-mercaptoacetamide (1.15 g, 12.6 mmol), and sodium carbonate (1.33 g, 12.6 mmol) were mixed in 100 mL of EtOH at room temperature overnight. The solvent was removed and the residue treated with water and EtOAc (50 mL each). The organic layer was separated and dried under high vacuum to yield the desired product (4.3 g, 100% yield). MS m/z calculated for (M+H)⁺ 381/383, found 381/383 (di-chloro pattern).

Step d: 5-Amino-4-(3,4-dichlorophenyl)-2-isopropylthieno[2,3-d]pyrimidine-6-carboxamide. 2-(5-cyano-6-(3,4-dichlorophenyl)-2-isopropylpyrimidin-4-ylthio)acetamide (4.38 g, 11.5 mmol) was placed in 100 mL of EtOH and Na metal (1.32 g, 57.5 mmol) was added in small portions while cooling the flask with an ice/water bath. The flask was placed under N₂ atmosphere and heated to reflux for 2 hours, cooled to room temperature, then solvent was removed and the residue was treated with water and EtOAc (100 mL each). The organic layer was washed with water, concentrated and dried under high vacuum. The solid obtained was sonicated in a small volume of EtOAc, filtered, and the filter cake washed with a hexane:EtOAc (2:1) mixture to yield the desired product (1.96 g, 45% yield). ¹H NMR (400 MHz, CDCl₃) δ ppm 1.41 (d, J=7.03 Hz, 6H) 3.21-3.44 (m, 1H) 5.42 (s, 2H) 6.08 (s, 2H) 7.48-7.56 (m, J=8.20 Hz, 1H) 7.65 (d, J=8.20 Hz, 1H) 7.81 (s, 1H). MS m/z calculated for (M+H)⁺ 381/383, found 381/383 (di-chloro pattern).

### 5.2.115 5-amino-4-(2-fluoro-3-hydroxyphenyl)-2-(1-hydroxy-2-methylpropan-2-ylamino)thieno[2,3-d]pyrimidine-6-carboxamide

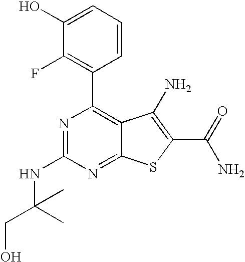

Step a: 4-(2-fluoro-3-methoxyphenyl)-6-hydroxy-2-(methylthio)pyrimidine-5-carbonitrile. 2-fluoro-3-methoxy benzaldehyde (25 g, 157 mmol), methyl carbamimidothioate hemisulfate (22.3 g, 78.5 mmol), ethyl 2-cyanoacetate (17.8 g, 157 mmol), and potassium carbonate (23.8 g, 173 mmol) were mixed in 600 mL dry EtOH under N₂ and heated at 75° C. overnight. The flask was cooled and the solvent removed. The residue was treated with water (300 mL) and EtOAc (2×500 mL). The organic layer was separated and concentrated to yield the desired product as a solid (22 g, 48% yield). ¹H NMR (300 MHz, DMSO-D6) δ ppm 2.31 (s, 3H) 3.87 (s, 3H) 6.98 (m, 1H) 7.3-7.2 (m, 2H).

Step b: 4-chloro-6-(2-fluoro-3-methoxyphenyl)-2-(methylthio)pyrimidine-5-carbonitrile. 4-(2-fluoro-3-methoxyphenyl)-6-hydroxy-2-(methylthio)pyrimidine-5-carbonitrile (22 g, 76 mmol) was placed in 1,4-dioxane (150 mL) and DMF was added (1.2 mL). POCl₃ (58 g, 378 mmol) was added and the resulting solution heated to reflux under N₂ for 4 hours. The reaction was cooled to room temperature and the solvent and excess POCl₃ removed. The residual POCl₃ was quenched with ice and a white solid was obtained and isolated by filtration and dried under high vacuum to yield the desired product (12.2 g, 52% yield). MS m/z calculated for (M+H)⁺ 310, found 310.

Step c: 2-(5-cyano-6-(2-fluoro-3-methoxyphenyl)-2-(methylthio)pyrimidin-4-ylthio)acetamide. 4-chloro-6-(2-fluoro-3-methoxyphenyl)-2-(methylthio)pyrimidine-5-carbonitrile (12 g, 38 mmol), 2-mercaptoacetamide (3.53 g, 38 mmol), and sodium carbonate (4.11 g, 38 mmol) were mixed in 250 mL of EtOH, placed under N₂ atm, and heated to reflux for 4 hours. The flask was cooled to room temperature and the excess solvent removed, 50 mL of water were added and, after sonication, the solid obtained was isolated by filtration and dried under high vacuum to yield the desired product (12.9 g, 91% yield). MS m/z calculated for (M+H)⁺ 365, found 365.

Step d: 5-amino-4-(2-fluoro-3-methoxyphenyl)-2-(methylsulfinyl)thieno[2,3-d]pyrimidine-6-carboxamide. 2-(5-cyano-6-(2-fluoro-3-methoxyphenyl)-2-(methylthio)pyrimidin-4-ylthio)acetamide (12.5 g, 34.3 mmol) was placed in 150 mL of anhydrous EtOH and Na metal (4.73, 206 mmol) added in small portions while cooling the flask with an ice/water bath. The flask was placed under N₂ atmosphere and heated to reflux for 3 hours, cooled to room temperature, and concentrated. The solid that formed upon concentration was isolated by filtration and dried under high vacuum. The solid was placed in 300 mL of CHCl₃, under N₂ and the mixture was stirred in a water/ice bath while mCPBA was added in small portions (6.8 g, 30 mmol). The reaction was warmed to room temperature and stirred for 3 hours. The reaction was quenched with 10% aq. NaHSO₃ (50 mL), the organic phase was washed with 10% aq. NaHCO₃, and the solvent evaporated to give the product as a yellow solid (5 g, 37% yield). MS m/z calculated for (M+H)⁺ 381, found 381.

Step e: 5-amino-4-(2-fluoro-3-methoxyphenyl)-2-(1-hydroxy-2-methylpropan-2-ylamino)thieno[2,3-d]pyrimidine-6-carboxamide. 5-amino-4-(2-fluoro-3-methoxyphenyl)-2-(methylsulfinyl)thieno[2,3-d]pyrimidine-6-carboxamide (0.4 g, 1 mmol) and 2-amino-2-methyl-1-propanol (0.5 g, 5.6 mmol) were dissolved in 3 mL of DMF and heated at 170° C. in the microwave for 45 minutes The sample was poured into water and extracted into EtOAc. MS m/z calculated for (M+H)⁺ 406, found 406.

Step f: 5-Amino-4-(2-fluoro-3-hydroxyphenyl)-2(1-hydroxy-2-methyl-2-ylamino)thieno[2,3-d]pyrimidine-6-carboxamide. 5-amino-4-(2-fluoro-3-methoxyphenyl)-2-(1-hydroxy-2-methylpropan-2-ylamino)thieno[2,3-d]pyrimidine-6-carboxamide (0.1 g, 0.25 mmol) was placed in CH₂Cl₂ and 1.5 mL of BBr₃ (1M in CH₂Cl₂) was added. The mixture was stirred at room temperature overnight then quenched by addition of 3 mL of water and the organic layer separated. The aqueous layer was neutralized with saturated NaHCO₃ solution and extracted with EtOAc (15 mL). The organic layer was purified by prep HPLC (Method 20-100%, 30 minutes, 20 mL/min) to yield the desired product (0.048 g, 51% yield). ¹H NMR (400 MHz, DMSO-D6) δ ppm 1.33 (s, 6H) 3.54 (d, Hz, 2H) 4.76-4.91 (m, 1H) 5.85 (s, 2H) 6.83-6.91 (m, 1H) 6.99 (s, 2H) 7.07-7.23 (m, 3H).

### 5.2.116 5-Amino-4-(2-fluoro-3-hydroxyphenyl)-2-((S)-1-hydroxypropan-2-ylamino)thieno[2,3-d]pyrimidine-6-carboxamide

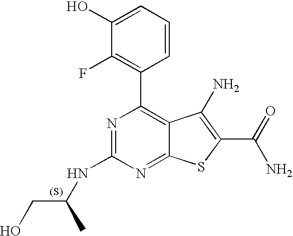

5-Amino-4-(2-fluoro-3-methoxyphenyl)-2-(methylsulfinyl)thieno[2,3-d]pyrimidine-6-carboxamide (0.5 g, 1.3 mmol) and (S)-2-aminopropan-1ol (0.14 g, 1.9 mmol) were dissolved in 3 mL of DMF and heated at 170° C. in the microwave for 45 minutes The sample was poured into water, extracted into EtOAc, and dried under high vacuum. The residue was placed in CH₂Cl₂ (50 mL) and BBr₃ (1M in CH₂Cl₂, 8 mL) was added. The mixture was stirred at room temperature overnight. The resulting reaction mixture was quenched by the addition of 10 mL of water and the aqueous layer separated. The aqueous layer was neutralized with saturated NaHCO₃ solution and extracted with EtOAc (15 mL). The organic layer was purified by prep HPLC (Method 20-100%, 30 minutes, 20 mL/min) to yield the desired product (0.02 g, 5.3% yield). ¹H NMR (400 MHz, DMSO-D6) δ ppm 1.13 (s, 3H) 3.48 (s, 1H) 3.96-4.07 (m, 1H) 4.72 (s, 1H) 5.67-5.94 (m, 6.25 Hz, 2H) 6.81-6.90 (m, 5.27 Hz, 1H) 6.98 (s, 2H) 7.08-7.21 (m, 2H) 7.60 (s, 1H). MS m/z calculated for (M+H)⁺ 378 found 378. >95% ee by chiral HPLC.

### 5.2.117 5-Amino-4-(2-fluoro-3-hydroxyphenyl)-2-((R)-1-hydroxypropan-2-ylamino)thieno[2,3-d]pyrimidine-6-carboxamide

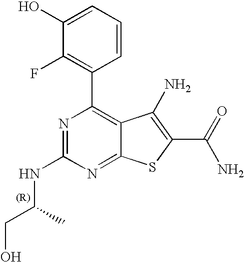

5-Amino-4-(2-fluoro-3-methoxyphenyl)-2-(methylsulfinyl)thieno[2,3-d]pyrimidine-6-carboxamide (1.0 g, 2.6 mmol) and (R)-2-aminopropan-1ol (0.29 g, 3.9 mmol) were dissolved in 3 mL of DMF and heated at 170° C. in the microwave for 45 minutes The sample was poured into water, extracted into EtOAc, and dried under high vacuum. The residue was placed in CH₂Cl₂ (50 mL) and BBr₃ (1 M in CH₂Cl₂, 16 mL) was added. The mixture was stirred at room temperature overnight. The resulting reaction mixture was quenched by the addition of 10 mL of water and the aqueous layer separated. The aqueous layer was neutralized with saturated NaHCO₃ solution and extracted with EtOAc (15 mL). The organic layer was purified by prep HPLC (Method 20-100%, 30 minutes, 20 mL/min) to yield the desired product (0.05 g, 13% yield). ¹H NMR (400 MHz, DMSO-D6) δ ppm 1.06-1.23 (m, 3H) 3.42-3.55 (m, 1H) 3.93-4.10 (m, 2H) 4.71 (s, 1H) 5.83 (s, 2H) 6.79-6.87 (m, 1H) 6.94-7.03 (m, 2H) 7.06-7.22 (m, 2H) 7.62 (s, 2H). MS m/z calculated for (M+H)⁺ 378, found 378. >95% ee by chiral analysis.

### 5.2.118 5-Amino-4-(2-fluoro-3-hydroxyphenyl)-2-((S)-1-hydroxybutan-2-ylamino)thieno[2,3-d]pyrimidine-6-carboxamide

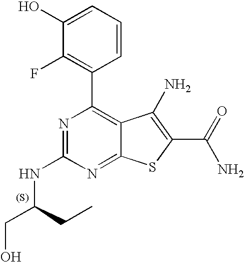

5-Amino-4-(2-fluoro-3-methoxyphenyl)-2-(methylsulfinyl)thieno[2,3-d]pyrimidine-6-carboxamide (0.5 g, 1.3 mmol) and (S)-2-aminobutan-1-ol (0.176 g, 1.97 mmol) were dissolved in 3 mL of DMF and heated at 170° C. in the microwave for 45 minutes The sample was poured into water, extracted into EtOAc, and dried under high vacuum. The residue was placed in CH₂Cl₂ (10 mL) and BBr₃ (1M in CH₂Cl₂, 5 mL) was added. The mixture was stirred at room temperature overnight then quenched by addition of 10 mL of water and the aqueous layer separated. The aqueous layer was neutralized with saturated NaHCO₃ solution and extracted with EtOAc (15 mL). The organic layer was purified using flash chromatography (silica gel, hexane:EtOAC 1:2-1:7) and prep HPLC (Method 20-100%, 30 minutes, 20 mL/min) to yield the desired product (0.011 g, 2% yield). ¹H NMR (400 MHz, CDCl₃) δ ppm 0.93-1.13 (m, J=14.45 Hz, 3H) 3.64-3.77 (m, 1H) 3.79-3.90 (m, 1H) 4.10 (s, 1H) 5.18 (s, 2H) 5.51 (s, 1H) 5.91 (s, 2H) 6.89-6.99 (m, 1H) 7.13-7.24 (m, J=5.08 Hz, 2H). MS m/z calculated for (M+H)⁺ 392, found 392. >95% e.e. by chiral analysis.

### 5.2.119 5-Amino-4-(2-fluoro-3-hydroxyphenyl)-2-((R)-1-hydroxybutan-2-ylamino)thieno[2,3-d]pyrimidine-6-carboxamide

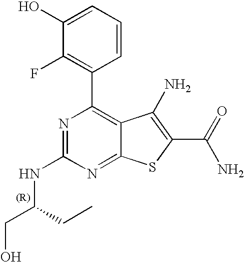

5-Amino-4-(2-fluoro-3-methoxyphenyl)-2-(methylsulfinyl)thieno[2,3-d]pyrimidine-6-carboxamide (1.0 g, 2.63 mmol) and (R)-2-aminobutan-1-ol (0.35 g, 3.95 mmol) were dissolved in 3 mL of DMF and heated at 170° C. in the microwave for 45 minutes The sample was poured into water, extracted into EtOAc, and dried under high vacuum. The residue was placed in CH₂Cl₂ (20 mL) and BBr₃ (1M in CH₂Cl₂, 7 mL) was added. The mixture was stirred at room temperature overnight. The resulting reaction mixture was quenched by addition of 10 mL of water and the aqueous layer separated. The aqueous layer was neutralized with saturated NaHCO₃ solution and extracted with EtOAc (20 mL). The organic layer was purified using flash chromatography (silica gel, hexane:EtOAC 1:2-1:7) and prep HPLC (Method 20-100%, 30 minutes, 20 mL/min) to yield the desired product (0.017 g, 1.6% yield). ¹H NMR (400 MHz, CDCl₃) δ ppm 0.96-1.13 (m, 3H) 1.67-1.81 (m, 1H) 3.66-3.74 (m, 2H) 3.79-3.90 (m, 2H) 5.19 (s, 2H) 5.52 (s, 1H) 5.91 (s, 2H) 6.87-7.00 (m, 1H) 7.13-7.22 (m, 2H). MS m/z calculated for (M+H)⁺ 392, found 392. >95% e.e. by chiral analysis.

### 5.2.120 5-amino-2-(cyclopropylamino)-4-(3-nitrophenyl)thiopheno[2,3-d]pyrimidine-6-carboxamide

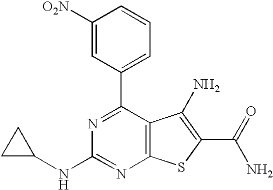

Step a: 2-(cyclopropylamino)-4-hydroxy-6-(3-nitrophenyl)pyrimidine-5-carbonitrile. 3-Nitrobenzaldehyde (5.0 g, 33 mmol), ethyl cyanoacetate (3.74 g, 33 mmol), and piperidine (2 drops), were refluxed in toluene (15 mL) with azeotropic removal of H₂O under N₂. After 1.5 hours the stirbar was removed and the volatiles removed on a rotary evaporator. To the resulting material was added sequentially: a stirbar, 1-cyclopropylguanidine hydrochloride (4.4 g, 33 mmol), K₂CO₃ (9.0 g, 66 mmol), and ethanol (50 mL). The resulting mixture was stirred vigorously and heated at 75° C. under a reflux condenser under N₂ for 1.5 hours. The reaction mixture was cooled in an ice bath and the resulting precipitate collected by filtration. The solids were washed with absolute EtOH and dissolved in hot water. The resulting solution was cooled to 0° C., acidified with 2 N HCl to pH 2. The resulting precipitate was collected by filtration, washed with ice water, and dried to give 7.5 g (76.5%) of 2-(cyclopropylamino)-4-hydroxy-6-(3-nitrophenyl)pyrimidine-5-carbonitrile as a light yellow solid. MS m/z calculated for (M+H)⁺ 298, found 298.

Step b: 4-chloro-2-(cyclopropylamino)-6-(3-nitrophenyl)pyrimidine-5-carbonitrile. To 2-(cyclopropylamino)-4-hydroxy-6-(3-nitrophenyl)pyrimidine-5-carbonitrile (5.0 g, 16.7 mmol) in dioxane (60 mL) was added POCl₃ (30 mL). The resulting reaction mixture was heated at 85° C. under a reflux condenser under N₂ for 4 hours and then cooled to room temperature. The volatiles were removed on a rotary evaporator. The resulting residue was dissolved in 1,4-dioxane (20 ml) and ice water was added with stirring. The resulting precipitate was collected by filtration, washed with H₂O, and dried. The solid was dissolved in EtOAc, silica gel was added, and the volatiles removed on a rotary evaporator. Flash chromatography (20% EtOAc/hexane) gave 3.5 g (66%) of 4-chloro-2-(cyclopropylamino)-6-(3-nitrophenyl)pyrimidine-5-carbonitrile as a yellow solid. MS m/z calculated for (M+H)⁺ 316, found 316.

Step c: 2-[5-cyano-2-(cyclopropylamino)-6-(3-nitrophenyl)pyrimidin-4-ylthio]acetamide. 4-chloro-2-(cyclopropylamino)-6-(3-nitrophenyl)pyrimidine-5-carbonitrile (4.0 g, 12.6 mmol), 2-mercaptoacetamide (1.3 g, 13.9 mmol), K₂CO₃ (3.5 g, 25.2 mmol), and ethanol (20 mL) were combined in a flask, stirred vigorously, and heated at 50° C. under a reflux condenser under N₂ for 16 hours. The reaction mixture was cooled to room temperature and 200 mL of ice water was added. The resulting solids were collected by vacuum filtration, washed once with H₂O, twice with 30% Et₂O/hexane, and dried under high vacuum at 50° C. to give 4.0 g (85%) of 2-[5-cyano-2-(cyclopropylamino)-6-(3-nitrophenyl)pyrimidin-4-ylthio]acetamide as a yellow solid. MS m/z calculated for (M+H)⁺ 370, found 370.

Step d: 5-amino-2-(cyclopropylamino)-4-(3-nitrophenyl)thiopheno[2,3-d]pyrimidine-6-carboxamide. A stirred suspension of 2-[5-cyano-2-(cyclopropylamino)-6-(3-nitrophenyl)pyrimidin-4-ylthio]acetamide (4.0 g, 10.8 mmol) in ethanol (60 mL) was cooled to 0° C. under N₂. Freshly prepared NaOEt (40 mmol) in ethanol (20 mL) was added and the resulting mixture stirred 2 hours while being slowly warmed to room temperature. The mixture was then heated at 45° C. for 2 hours. HCl (45 mmol) in 200 mL of ice water was added with vigorous stirring. The resulting solids were collected by vacuum filtration, washed once with H₂O, once with 20% Et₂O/hexane, and then dried under high vacuum. The solid was dissolved in EtOAc, silica gel was added, and the volatiles removed on a rotary evaporator. Flash chromatography (80-20% EtOAc/hexane) gave 2.3 g (57.5%) of 5-amino-2-(cyclopropylamino)-4-(3-nitrophenyl)thiopheno[2,3-d]pyrimidine-6-carboxamide as a yellow solid. ¹H NMR (300 MHz, DMSO-d₆): δ ppm 0.50-0.54 (m, 2H) 0.70-0.72 (m, 2H) 2.80-2.83 (m, 1H) 6.10 (s, 2H) 7.06 (s, 2H) 7.77-7.82 (m, 1H) 8.02-8.04 ((d, J=6 Hz, 2H) 8.38 (s, 1H) 8.4 (s, 1H). MS m/z calculated for (M+H)⁺ 371, found 371. Analytical HPLC retention time: 5.04 minutes (method I).

### 5.2.121 5-amino-4-(3-aminophenyl)-2-(cyclopropylamino)thiopheno[2,3-d]pyrimidine-6-carboxamide

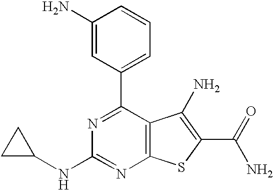

5-amino-2-(cyclopropylamino)-4-(3-nitrophenyl)thiopheno[2,3-d]pyrimidine-6-carboxamide (0.500 g, 1.34 mmol) was stirred in EtOH (15 mL) and EtOAc (15 mL) with Pd/C under a balloon of hydrogen for 16 hours. The reaction mixture was filtered through a pad of celite. The resulting solution was concentrated on a rotary evaporator and the crude product dissolved in DMSO (2 mL) and purified using reverse-phase preparative HPLC (20-80% acetonitrile, 0.1% TFA, 30 minutes). The appropriate fractions were combined and concentrated on a rotary evaporator. The residue was dissolved in ethyl acetate (100 mL), washed with aqueous NaHCO₃, and dried over Na₂SO₄. The solvent was removed in vacuo to give 5-amino-4-(3-aminophenyl)-2-(cyclopropylamino)thiopheno[2,3-d]pyrimidine-6-carboxamide (0.400 g, 87%) as a yellow solid. ¹H NMR (300 MHz, DMSO-d6): δ ppm 0.50-0.54 (m, 2H) 0.70-0.72 (m, 2H) 2.80-2.83 (m, 1H) 5.40 (s, 2H) 6.10 (s, 2H) 6.60-6.62 (d, J=6 Hz, 1H) 6.67 (s, 1H) 6.70-6.73 (d, J=9 Hz, 1H) 6.96 (s, 2H) 7.15-7.20 (t, 1H) 7.91 (s, 1H). MS m/z calculated for (M+H)⁺ 341, found 341. Analytical HPLC retention time: 3.74 minutes (method I).

### 5.2.122 5-amino-2-(cyclopropylamino)-4-[3-(2,2,2-trifluoroacetylamino)phenyl]thiopheno[2,3-d]pyrimidine-6-carboxamide

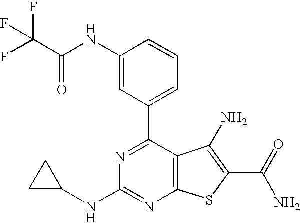

A solution of 5-amino-4-(3-aminophenyl)-2-(cyclopropylamino)thiopheno[2,3-d]pyrimidine-6-carboxamide (0.300 g, 0.87 mmol) in CH₂Cl₂ (10 mL) and pyridine (1 mL) was cooled to 0° C. and trifluoroacetic anhydride (0.200 g, 0.95 mmol) was added dropwise. The reaction mixture was stirred at room temperature for 2 hours then diluted with EtOAc (50 mL). The resulting mixture was washed with saturated NaHCO₃, brine, and dried over Na₂SO₄. Volatiles were removed on a rotary evaporator and the crude product was dissolved in DMSO (1 mL) and purified using reverse-phase preparative HPLC (10-80% acetonitrile, 0.1% TFA, 30 minutes). The appropriate fractions were combined and concentrated on a rotary evaporator. The residues were dissolved in ethyl acetate (20 mL), washed with aqueous NaHCO₃, and dried over Na₂SO₄. The solvent was removed in vacuo to give 0.160 g (48%) of 5-amino-2-(cyclopropylamino)-4-[3-(2,2,2-trifluoroacetylamino)phenyl]thiopheno[2,3-d]pyrimidine-6-carboxamide as a yellow solid. ¹H NMR (300 MHz, DMSO-d6) δ ppm 0.50-0.54 (m, 2H) 0.70-0.72 (m, 2H) 2.80-2.83 (m, 1H) 6.10 (s, 2H) 7.06 (s, 2H) 7.28-7.30 (d, J=6 Hz, 1H) 7.52-7.58 (t, 1H) 7.79-7.81 (d, J=6 Hz, 1H) 7.89 (s, 1H) 8.00 (s, 1H) 10.26 (s, 1H). MS m/z calculated for (M+H)⁺ 437, found 437. Analytical HPLC retention time: 5.14 minutes (method I).

### 5.2.123 5-amino-2-(cyclopropylamino)-4-{3-[(2-phenylethyl)amino]phenyl}thiopheno[2,3-d]pyrimidine-6-carboxamide

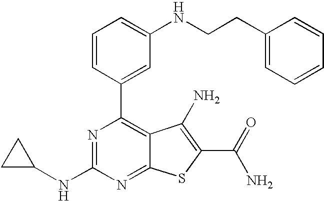

To a solution of 5-amino-4-(3-aminophenyl)-2-(cyclopropylamino)thiopheno[2,3-d]pyrimidine-6-carboxamide (0.200 g, 0.58 mmol), phenylacetaldehyde (0.072 g, 0.58 mmol) and HOAc (2 drops) in DMF (9 mL) was added sodium triacetoxyborohydride (0.373 g, 1.76 mmol). The reaction mixture was stirred under N₂ for 16 hours. Saturated, aqueous NaHCO₃ was added and the mixture extracted with EtOAc. The organics were washed once with brine, dried over MgSO₄, filtered, and concentrated on a rotary evaporator. The crude product was dissolved in DMSO (2 mL) and purified using reverse-phase preparative HPLC (30-80% acetonitrile, 0.1% TFA, 30 minutes). The appropriate fractions were combined and concentrated on a rotary evaporator. The residue was dissolved in ethyl acetate (100 mL), washed with aqueous NaHCO₃, and dried over Na₂SO₄. The solvent was removed in vacuo to give 140 mg (38%) of 5-amino-2-(cyclopropylamino)-4-{3-[(2-phenylethyl)amino]phenyl}thiopheno[2,3-d]pyrimidine-6-carboxamide. ¹H NMR (400 MHz, METHANOL-D4) δ ppm 0.55-0.61 (m, 2H) 0.77-0.84 (m, 2H) 2.77-2.84 (m, 1H) 2.89 (t, J=7.42 Hz, 2H) 3.35-3.41 (m, 2H) 6.71-6.75 (m, 2H) 6.78-6.82 (m, 1H) 7.13-7.18 (m, 1H) 7.22-7.31 (m, 5H). MS m/z calculated for (M+H)⁺ 445, found 445. Analytical HPLC retention time: 5.88 minutes (method I).

### 5.2.124 5-amino-2-(cyclopropylamino)-4-{3-[(phenylamino)carbonylamino]phenyl}thiopheno[2,3-d]pyrimidine-6-carboxamide

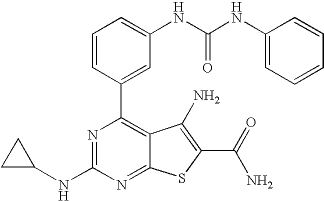

5-amino-4-(3-aminophenyl)-2-(cyclopropylamino)thiopheno[2,3-d]pyrimidine-6-carboxamide (0.150 g, 0.44 mmol) and phenyl isocyanate (0.053 g, 0.44 mmol) were combined in THF (9 mL) with stirring under N₂ for 16 hours. The resulting mixture was concentrated on a rotary evaporator and the crude product was dissolved in DMSO (2 mL) and purified using reverse-phase preparative HPLC (30-80% acetonitrile, 0.1% TFA, 30 minutes). The appropriate fractions were combined and concentrated on a rotary evaporator. The residue was dissolved in ethyl acetate (100 mL), washed with aqueous NaHCO₃, and dried over Na₂SO₄. The solvent was removed in vacuo to give 80 mg (40%) of 5-amino-2-(cyclopropylamino)-4-{3-[(phenylamino)carbonylamino]phenyl}thiopheno[2,3-d]pyrimidine-6-carboxamide. ¹H NMR (400 MHz, DMSO-D6) δ ppm 0.49-0.58 (m, 2H) 0.67-0.77 (m, 2H) 2.77-2.88 (m, 1H) 6.09 (s, 2H) 6.94-7.02 (m, 3H) 7.16 (d, J=7.42 Hz, 1H) 7.24-7.32 (m, 2H) 7.42-7.50 (m, 3H) 7.59 (d, J=7.03 Hz, 1H) 7.70 (t, J=1.76 Hz, 1H) 7.94 (s, 1H) 8.73 (s, 1H) 8.91 (s, 1H). MS m/z calculated for (M+H)⁺ 460, found 460. Analytical HPLC retention time: 5.22 minutes (method I).

### 5.2.125 5-amino-4-[3-({[4-chloro-3-(trifluoromethyl)phenyl]amino}carbonylamino)phenyl]-2-(cyclopropylamino)thiopheno[2,3-d]pyrimidine-6-carboxamide

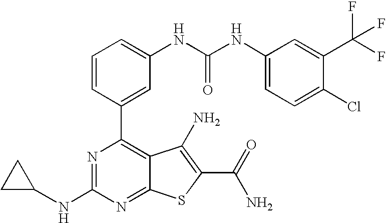

5-amino-4-(3-aminophenyl)-2-(cyclopropylamino)thiopheno[2,3-d]pyrimidine-6-carboxamide (0.150 g, 0.44 mmol) and 4-chloro-3-(trifluoromethyl)phenyl isocyanate (0.100 g, 0.44 mmol) were combined in THF (9 mL) with stirring under N₂ for 16 hours. Volatiles were removed on a rotary evaporator and the crude product was dissolved in DMSO (2 mL) and purified using reverse-phase preparative HPLC (30-80% acetonitrile, 0.1% TFA, 30 minutes). The appropriate fractions were combined and concentrated on a rotary evaporator. The residue was dissolved in ethyl acetate (100 mL), washed with aqueous NaHCO₃, and dried over Na₂SO₄. The solvent was removed in vacuo to give 95 mg (38.5%) of 5-amino-4-[3-({[4-chloro-3-(trifluoromethyl)phenyl]amino}carbonylamino)phenyl]-2-(cyclopropylamino)thiopheno[2,3-d]pyrimidine-6-carboxamide. ¹H NMR (400 MHz, DMSO-D6) δ ppm 0.54 (m, 2H) 0.71 (d, J=5.08 Hz, 2H) 2.74-2.92 (m, 1H) 6.07 (s, 2H) 6.98 (s, 2H) 7.19 (d, J=7.03 Hz, 1H) 7.48 (t, J=7.81 Hz, 1H) 7.56-7.69 (m, 3H) 7.70 (s, 1H) 7.94 (s, 1H) 8.10 (s, 1H) 9.09 (s, 1H) 9.23 (s, 1H). MS m/z calculated for (M+H)⁺ 562, found 562. Analytical HPLC retention time: 6.11 minutes (method I).

### 5.2.126 5-amino-2-(cyclopropylamino)-4-[3-({[4-(trifluoromethyl)phenyl]amino}carbonylamino)phenyl]thiopheno[2,3-d]pyrimidine-6-carboxamide

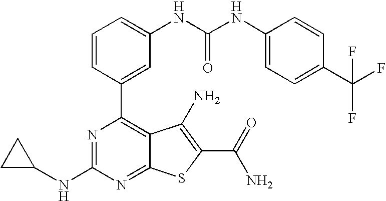

5-amino-4-(3-aminophenyl)-2-(cyclopropylamino)thiopheno[2,3-d]pyrimidine-6-carboxamide (0.100 g, 0.29 mmol) and 4-(trifluoromethyl)phenyl isocyanate (0.055 g, 0.29 mmol) were dissolved in THF (9 mL) with stirring under N₂ for 3 hours. Volatiles were removed on a rotary evaporator and the crude product was dissolved in DMSO (2 mL) and purified using reverse-phase preparative HPLC (30-80% acetonitrile, 0.1% TFA, 30 minutes). The appropriate fractions were combined and concentrated on a rotary evaporator. The residue was dissolved in ethyl acetate (100 mL), washed with aqueous NaHCO₃, and dried over Na₂SO₄. The solvent was removed in vacuo to give 85 mg (55%) of 5-amino-2-(cyclopropylamino)-4-[3-({[4-(trifluoromethyl)phenyl]amino}carbonylamino)phenyl]thiopheno[2,3-d]pyrimidine-6-carboxamide. ¹H NMR (400 MHz, DMSO-D6) δ ppm 0.56 (m, 2H) 0.71 (d, J=5.08 Hz, 2 H) 2.74-2.92 (m, 1H) 6.12 (s, 2H) 7.02 (s, 2H) 7.19 (d, J=7.03 Hz, 1H) 7.48 (t, J=7.81 Hz, 1H) 7.62-7.74 (m, 6H) 8.10 (s, 1H) 9.16 (s, 1H) 9.29 (s, 1H). MS m/z calculated for (M+H)⁺ 628, found 628. Analytical HPLC retention time: 5.83 minutes (method I).

### 5.2.127 5-amino-2-(cyclopropylamino)-4-(3-{[(4-methylphenyl)amino]carbonylamino}phenyl)thiopheno[2,3-d]pyrimidine-6-carboxamide

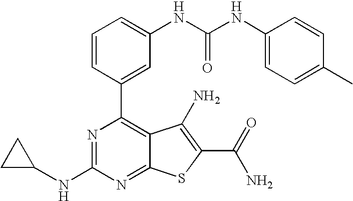

5-amino-4-(3-aminophenyl)-2-(cyclopropylamino)thiopheno[2,3-d]pyrimidine-6-carboxamide (0.070 g, 0.20 mmol) and p-tolylisocyanate (0.025 g, 0.20 mmol) were dissolved in THF (9 mL) with stirring under N₂ for 3 hours. Volatiles were removed on a rotary evaporator and the crude product was dissolved in DMSO (2 mL) and purified using reverse-phase preparative HPLC (30-80% acetonitrile, 0.1% TFA, 30 minutes). The appropriate fractions were combined and concentrated on a rotary evaporator. The residue was dissolved in ethyl acetate (100 mL), washed with aqueous NaHCO₃, and dried over Na₂SO₄. The solvent was removed in vacuo to give 55 mg (57%) of 5-amino-2-(cyclopropylamino)-4-(3-{[(4-methylphenyl)amino]carbonylamino}phenyl)thiopheno[2,3-d]pyrimidine-6-carboxamide. ¹H NMR (300 MHz, DMSO-D6) δ ppm 0.44-0.60 (m, 2H) 0.64-0.79 (m, 2H) 2.24 (s, 3H) 2.74-2.92 (m, 1H) 6.09 (s, 2H) 7.00 (s, 2H) 7.07 (s, 1H) 7.10 (s, 1H) 7.14 (d, J=7.14 Hz, 1H) 7.32 (s, 1H) 7.35 (s, 1H) 7.45 (t, J=7.97 Hz, 1H) 7.58 (d, J=7.42 Hz, 1H) 7.69 (s, 1H) 7.97 (s, 1H) 8.68 (s, 1H) 8.92 (s, 1H). MS m/z calculated for (M+H)⁺ 474, found 474. Analytical HPLC retention time: 5.44 minutes (method I).

### 5.2.128 5-amino-2-(cyclopropylamino)-4-(3-{[(4-fluorophenyl)amino]carbonylamino}phenyl)thiopheno[2,3-d]pyrimidine-6-carboxamide

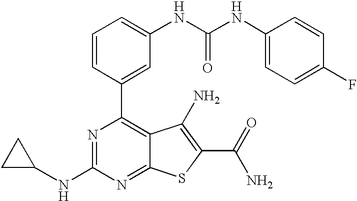

5-amino-4-(3-aminophenyl)-2-(cyclopropylamino)thiopheno[2,3-d]pyrimidine-6-carboxamide (0.110 g, 0.320 mmol) and 4-fluorophenyl isocyanate (0.044 g, 0.320 mmol) were dissolved in THF (9 mL) with stirring under N₂ for 3 hours. Volatiles were removed on a rotary evaporator and the crude product was dissolved in DMSO (2 mL) and purified using reverse-phase preparative HPLC (30-80% acetonitrile, 0.1% TFA, 30 minutes). The appropriate fractions were combined and concentrated on a rotary evaporator. The residue was dissolved in ethyl acetate (100 mL), washed with aqueous NaHCO₃, and dried over Na₂SO₄. The solvent was removed in vacuo to give 100 mg (71%) of 5-amino-2-(cyclopropylamino)-4-(3-{[(4-fluorophenyl)amino]carbonylamino}phenyl)thiopheno[2,3-d]pyrimidine-6-carboxamide. ¹H NMR (400 MHz, DMSO-D6) δ ppm 0.54 (s, 2H) 0.72 (d, J=5.47 Hz, 2H) 2.82 (s, 1H) 6.10 (s, 2H) 7.01 (s, 2H) 7.14 (q, J=8.46 Hz, 3H) 7.37-7.54 (m, 3H) 7.59 (d, J=7.03 Hz, 1H) 7.70 (s, 1H) 7.97 (s, 1H) 8.81 (s, 1H) 8.94 (s, 1H). MS m/z calculated for (M+H)⁺ 478, found 478. Analytical HPLC retention time: 5.28 minutes (method I).

### 5.2.129 5-amino-2-[(3-amino-2,2-dimethylpropyl)amino]-4-(3, 4-dichlorophenyl)thiopheno[2,3-d]pyrimidine-6-carboxamide

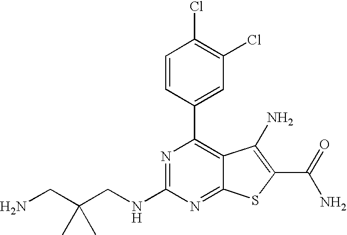

Step a: 5-amino-4-(3,4-dichlorophenyl)-2-({3-[(tert-butoxy)carbonylamino]-2,2-dimethylpropyl}amino)thiopheno[2,3-d]pyrimidine-6-carboxamide. 5-amino-4-(3,4-dichlorophenyl)-2-(methylsulfinyl)thiopheno[2,3-d]pyrimidine-6-carboxamide (0.250 g, 0.62 mmol) and 1-Boc-amino-2,3-dimethyl-1,3-propyldiamine (0.383 g, 1.9 mmol) were dissolved in THF (20 mL) and heated at reflux for 18 hours. Volatiles were removed on a rotary evaporator and flash chromatography (40% EtOAc/hexane) gave 100 mg (42%) of 5-amino-4-(3,4-dichlorophenyl)-2-({3-[(tert-butoxy)carbonylamino]-2,2-dimethylpropyl}amino)thiopheno[2,3-d]pyrimidine-6-carboxamide as a light yellow solid. MS m/z calculated for (M+H)⁺ 539, found 539.

Step b: 5-amino-2-[(3-amino-2,2-dimethylpropyl)amino]-4-(3,4-dichlorophenyl)thiopheno[2,3-d]pyrimidine-6-carboxamide. A solution of 5-amino-4-(3,4-dichlorophenyl)-2-({3-[(tert-butoxy)carbonylamino]-2,2-dimethylpropyl}amino) thiopheno[2,3-d]pyrimidine-6-carboxamide (0.100 g, 0.18 mmol) dissolved in CH₂Cl₂ (10 mL) was cooled to 0° C. Excess 4 M HCl in dioxane (2 mL) was added and the reaction was allowed to warm to room temperature and stir for 2 hours. Volatiles were removed on a rotary evaporator and the crude product was dissolved in DMSO (2 mL) and purified using reverse-phase preparative HPLC (10-80% acetonitrile, 0.1% TFA, 30 minutes). The appropriate fractions were combined and concentrated on a rotary evaporator. The residue was dissolved in ethyl acetate (100 mL), washed with aqueous NaHCO₃, and dried over Na₂SO₄. The solvent was removed in vacuo to give 50 mg (41%) of 5-amino-2-[(3-amino-2,2-dimethylpropyl)amino]-4-(3,4-dichlorophenyl)thiopheno[2,3-d]pyrimidine-6-carboxamide. ¹H NMR (300 MHz, DMSO-D6) δ ppm 0.92 (s, 6H) 2.58 (s, 2H) 3.17 (s, 2H) 6.16 (s, 4H) 7.04 (s, 2H) 7.58 (d, J=9.34 Hz, 1H) 7.80 (d, J=8.52 Hz, 1H) 7.86 (s, 1H) 7.96 (s, 1H). MS m/z calculated for (M+H)⁺ 440, found 440. Analytical HPLC retention time: 3.72 minutes (method I).

### 5.2.130 5-amino-4-(3,4-dichlorophenyl)-2-(carbamoylmethyl)thiopheno[2,3-d]pyrimidine-6-carboxamide and 2-[5-amino-4-(3, 4-dichlorophenyl)-6-carbamoylthiopheno[2,3-d]pyrimidin-2-yl]acetic acid

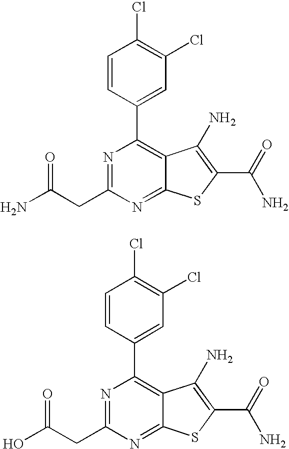

Step a: ethyl 2-[5-amino-4-(3,4-dichlorophenyl)-6-carbamoylthiopheno[2,3-d]pyrimidin-2-yl]acetate. To a suspension of ethylmalonate potassium salt (5 g, 29 mmol) in water (15 mL) was added 6 N HCl to pH 2. The mixture was extracted with EtOAc. The combined organic layer was washed with brine, dried, filtered and concentrated. The residue was dissolved in EtOH (15 mL) and magnesium ethoxide (1.5 g, 13.2 mmol) was added over 5 minutes The reaction mixture was stirred at room temperature for 16 hours. Volatiles were removed on a rotary evaporator. The residuals were triturated with isopropanol and dried under high vacuum. The residue (1.4 g, 5 mmol) was dissolved in dry THF (20 mL) with stirring at room temperature under N₂. NaH (0.200 g, 5 mmol) was added in portions, after 10 minutes 5-amino-4-(3,4-dichlorophenyl)-2-(methylsulfinyl)thiopheno[2,3-d]pyrimidine-6-carboxamide (0.400 g, 1.0 mmol) in dry THF (10 mL) was added and the mixture was refluxed for 18 hours. Volatiles were removed on a rotary evaporator and flash chromatography (40-50% EtOAc/hexane) gave 250 mg (55%) of ethyl 2-[5-amino-4-(3,4-dichlorophenyl)-6-carbamoylthiopheno[2,3-d]pyrimidin-2-yl]acetate as a light yellow solid. MS m/z calculated for (M+H)⁺ 425, found 425.

Step b: 5-amino-4-(3,4-dichlorophenyl)-2-(carbamoylmethyl)thiopheno[2,3-d]pyrimidine-6-carboxamide. A solution of ethyl 2-[5-amino-4-(3,4-dichlorophenyl)-6-carbamoylthiopheno[2,3-d]pyrimidin-2-yl]acetate (0.250 g, 0.59 mmol)) in EtOH (14 mL) in a 200 ml high pressure flask was cooled to −75° C. under N₂. NH₃ gas (2 mL) was bubbled in for two minutes then the high pressure flask was tightly closed and warmed to room temperature with stirring overnight. Volatiles were removed on a rotary evaporator and the crude product was dissolved in DMSO (2 mL) and purified using reverse-phase preparative HPLC (10-80% acetonitrile, 0.1% TFA, 30 minutes). The appropriate fractions were combined and concentrated on a rotary evaporator. The residues were dissolved in ethyl acetate (100 mL), washed with aqueous NaHCO₃, and dried over Na₂SO₄. The solvent was removed in vacuo to give two products.

5-amino-4-(3,4-dichlorophenyl)-2-(carbamoylmethyl)thiopheno[2,3-d]pyrimidine-6-carboxamide (50 mg, 25%) as a yellow solid. ¹H NMR (400 MHz, DMSO-D6) δ ppm 3.85 (s, 2H) 6.36 (s, 2H) 7.10 (s, 1H) 7.42 (s, 2H) 7.56 (s, 1H) 7.65 (dd, J=8.20, 1.95 Hz, 1H) 7.84 (d, J=8.20 Hz, 1H) 7.92 (d, J=1.95 Hz, 1H). MS m/z calculated for (M+H)⁺ 397, found 397. Analytical HPLC retention time: 4.41 minutes (method I).

2-[5-amino-4-(3,4-dichlorophenyl)-6-carbamoylthiopheno[2,3-d]pyrimidin-2-yl]acetic acid (50 mg, 25%) as a yellow solid. ¹H NMR (400 MHz, DMSO-D6) δ ppm 4.02 (s, 2H) 6.37 (s, 2H) 7.44 (s, 2H) 7.66 (dd, J=8.20, 1.95 Hz, 1H) 7.84 (d, J=8.20 Hz, 1H) 7.93 (d, J=1.95 Hz, 1H) 12.69 (s, 1H). MS m/z calculated for (M+H)⁺ 398, found 398. Analytical HPLC retention time: 4.84 minutes (method I).

### 5.2.131 5-amino-2-[(2-amino-tert-butyl)amino]-4-(3,4-dichlorophenyl)thiopheno[2,3-d]pyrimidine-6-carboxamide

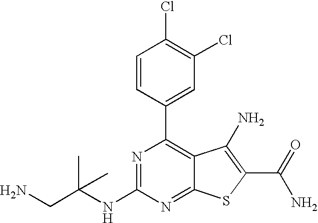

Step a: 5-amino-4-(3,4-dichlorophenyl)-2-({2-[(tert-butoxy)carbonylamino]-tert-butyl}amino)thiopheno[2,3-d]pyrimidine-6-carboxamide. 5-amino-4-(3,4-dichlorophenyl)-2-(methylsulfinyl)thiopheno[2,3-d]pyrimidine-6-carboxamide (0.400 g, 1.0 mmol), N-(2-amino-2-methylpropyl)(tert-butoxy)carboxamide (0.225 g, 1.2 mmol) and triethylamine (0.5 mL, 3 mmol) were dissolved in THF (20 mL) and heated at reflux for 18 hours. Volatiles were removed on a rotary evaporator and flash chromatography (40% EtOAc/hexane) gave 150 mg (30%) of 5-amino-4-(3,4-dichlorophenyl)-2-({2-[(tert-butoxy)carbonylamino]-tert-butyl}amino)thiopheno[2,3-d]pyrimidine-6-carboxamide as a light yellow solid. MS m/z calculated for (M+H)⁺ 525, found 525.

Step b: 5-amino-2-[(2-amino-tert-butyl)amino]-4-(3,4-dichlorophenyl)thiopheno[2,3-d]pyrimidine-6-carboxamide. 5-amino-4-(3,4-dichlorophenyl)-2-({2-[(tert-butoxy)carbonylamino]-tert-butyl}amino)thiopheno[2,3-d]pyrimidine-6-carboxamide (0.100 g, 0.18 mmol) dissolved in CH₂Cl₂ (10 mL) was cooled to 0° C. Excess 4 M HCl in dioxane (2 mL) was added and the reaction was allowed to warm to room temperature and stirred for 2 hours. Volatiles were removed on a rotary evaporator and the crude product was dissolved in DMSO (2 mL) and purified using reverse-phase preparative HPLC (10-80% acetonitrile, 0.1% TFA, 30 minutes). The appropriate fractions were combined and concentrated on a rotary evaporator. The residue was dissolved in ethyl acetate (100 mL), washed with aqueous NaHCO₃, and dried over Na₂SO₄. The solvent was removed in vacuo to give 30 mg (25%) of 5-amino-2-[(2-amino-tert-butyl)amino]-4-(3,4-dichlorophenyl)thiopheno[2,3-d]pyrimidine-6-carboxamide as a yellow solid. ¹H NMR (400 MHz, DMSO-D6) δ ppm 1.35 (s, 6H) 2.90 (s, 2H) 6.18 (s, 2H) 7.02 (s, 2H) 7.27 (s, 1H) 7.59 (d, J=8.20 Hz, 1H) 7.80 (d, J=8.20 Hz, 1H) 7.85 (d, J=1.95 Hz, 1H). MS m/z calculated for (M+H)⁺ 426, found 426. Analytical HPLC retention time: 3.53 minutes (method I).

### 5.2.132 5-amino-2-[(2-hydroxy-tert-butyl)amino]-4-[3-({[4-(trifluoromethyl)phenyl]amino}carbonylamino)phenyl]thiopheno[2,3-d]pyrimidine-6-carboxamide

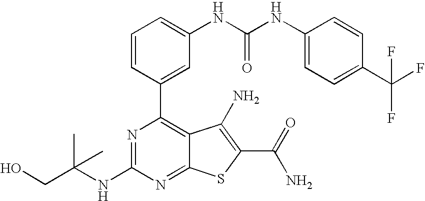

Step a: 4-hydroxy-2-methylthio-6-(3-nitrophenyl)pyrimidine-5-carbonitrile. 3-Nitrobenzaldehyde (5.0 g, 33 mmol), ethyl cyanoacetate (3.74 g, 33 mmol), and piperidine (2 drop) were refluxed in toluene (15 mL) with azeotropic removal of H₂O under N₂. After 1.5 hours the stirbar was removed and the volatiles removed on a rotary evaporator. To the resulting material was added sequentially: a stirbar, s-methylisothiouronium sulfate (4.58 g, 16.5 mmol), K₂CO₃ (9.0 g, 66 mmol), and ethanol (50 mL). The resulting mixture was stirred vigorously and heated at 75° C. under a reflux condenser under N₂ for 4 hours. The reaction mixture was cooled in ice bath and resulting precipitate was collected by filtration, washed with absolute EtOH and dissolved in hot water. The resulting solution was cooled to 0° C. and acidified with 2 N HCl to pH 2. The resulting precipitate was filtered, washed with ice water and dried to give 5.5 g (57.5%) of 4-hydroxy-2-methylthio-6-(3-nitrophenyl)pyrimidine-5-carbonitrile as a light yellow solid. MS m/z calculated for (M+H)⁺ 289, found 289.

Step b: 4-chloro-2-methylthio-6-(3-nitrophenyl)pyrimidine-5-carbonitrile. To 4-hydroxy-2-methylthio-6-(3-nitrophenyl)pyrimidine-5-carbonitrile (5.0 g, 17.3 mmol) in dioxane (60 mL) was added POCl₃ (30 mL). The resulting reaction mixture was heated at 85° C. under a reflux condenser under N₂ for 4 hours and then cooled to room temperature. The volatiles were removed on a rotary evaporator. The resulting residue was dissolved in 1,4-dioxane (20 ml) and ice water was added with stirring. The resulting precipitate was collected by filtration, washed with H₂O and dried. The solid was dissolved in EtOAc, silica gel was added, and the volatiles removed on a rotary evaporator. Flash chromatography (20% EtOAc/hexane) gave 3.7 g (69%) of 4-chloro-2-methylthio-6-(3-nitrophenyl)pyrimidine-5-carbonitrile as a yellow solid. ¹H NMR (400 MHz, CHLOROFORM-D) δ ppm 2.69 (s, 3H) 7.78 (t, J=8.00 Hz, 1H) 8.41 (dd, J=7.81, 1.56 Hz, 1H) 8.44-8.51 (m, 1H) 8.90 (s, 1H).

Step c: 2-[5-cyano-2-methylthio-6-(3-nitrophenyl)pyrimidin-4-ylthio]acetamide. 4-chloro-2-methylthio-6-(3-nitrophenyl)pyrimidine-5-carbonitrile (3.7 g, 12.5 mmol), 2-mercaptoacetamide (1.3 g, 13.9 mmol), K₂CO₃ (3.5 g, 25.2 mmol), and ethanol (20 mL) were combined in a flask, stirred vigorously, and heated at 50° C. under a reflux condenser under N₂ for 16 hours. The reaction mixture was cooled to room temperature and 200 mL of ice water was added. The resulting solids were collected by vacuum filtration, washed once with H₂O, twice with 30% Et₂O/hexane, and dried under high vacuum at 50° C. to give 4.0 g (85%) of 2-[5-cyano-2-methylthio-6-(3-nitrophenyl)pyrimidin-4-ylthio]acetamide as a very light yellow solid. MS m/z calculated for (M+H)⁺ 362, found 362.

Step d: 5-amino-2-methylthio-4-(3-nitrophenyl)thiopheno[2,3-d]pyrimidine-6-carboxamide. A stirred suspension of 2-[5-cyano-2-methylthio-6-(3-nitrophenyl)pyrimidin-4-ylthio]acetamide (4.0 g, 11.0 mmol) in ethanol (60 mL) was cooled to 0° C. under N₂. Freshly prepared NaOEt (40 mmol) in ethanol (20 mL) was added and the resulting mixture stirred 2 hours while being slowly warmed to room temperature. The mixture was then heated at 45° C. for 2 hours. HCl (45 mmol) in 200 mL of ice water was added with vigorous stirring. The resulting solids were collected by vacuum filtration, washed once with H₂O, once with 20% Et₂O/hexane, and then dried under high vacuum. The solid was dissolved in EtAOc and silica gel was added and the volatiles removed on a rotary evaporator. Flash chromatography (80-20% EtOAc/hexane) gave 2.4 g (60%) of 5-amino-2-methylthio-4-(3-nitrophenyl)thiopheno[2,3-d]pyrimidine-6-carboxamide as a yellow solid. ¹H NMR (300 MHz, DMSO-D6) δ ppm 2.64 (s, 3H) 6.28 (s, 2H) 7.41 (s, 2H) 7.76-7.99 (m, 1H) 8.15 (d, J=7.69 Hz, 1H) 8.38-8.61 (m, 2H). MS m/z calculated for (M+H)⁺ 362, found 362.

Step e: 5-amino-4-(3-aminophenyl)-2-methylthiothiopheno[2,3-d]pyrimidine-6-carboxamide. A mixture of 5-amino-2-methylthio-4-(3-nitrophenyl)thiopheno[2,3-d]pyrimidine-6-carboxamide (2.5 g, 6.9 mmol), EtOH (50 mL), water (50 mL) and iron powder (1.1 g, 20.8 mmol) was stirred and brought to reflux during which time a flow of N₂ was passed through the mixture to degas it of oxygen for 20 minutes The refluxing mixture was treated with HCl (0.2 mL) and stirred overnight. The reaction mixture was cooled and celite (2.0 g) was added, stirred for 10 minutes and filtered through a pad of celite. Volatiles were removed on a rotary evaporator and extracted with EtOAc (2×100 mL). Volatiles were removed on a rotary evaporator and the crude product was dissolved in DMSO (2 mL) and purified using reverse-phase preparative HPLC (20-80% acetonitrile, 0.1% TFA, 30 minutes). The appropriate fractions were combined and concentrated on a rotary evaporator. The residue was dissolved in ethyl acetate (100 mL), washed with aqueous NaHCO₃, and dried over Na₂SO₄. The solvent was removed in vacuo to give 5-amino-4-(3-aminophenyl)-2-methylthiothiopheno[2,3-d]pyrimidine-6-carboxamide (1.2 g, 55%) as a yellow solid. MS m/z calculated for (M+H)⁺ 332, found 332.

Step f: 5-amino-2-methylthio-4-[3-({[4-(trifluoromethyl)phenyl]amino}carbonylamino)phenyl]thiopheno[2,3-d]pyrimidine-6-carboxamide. 5-amino-4-(3-aminophenyl)-2-methylthiothiopheno[2,3-d]pyrimidine-6-carboxamide (0.700 g, 2.15 mmol) and 4-(trifluoromethyl)phenyl isocyanate (0.504 g, 2.28 mmol) were dissolved in THF (15 mL) and heated at reflux with stirring under N₂ for 3 hours. The reaction mixture was cooled and the precipitate was collected by filtration to give 5-amino-2-methylthio-4-[3-({[4-(trifluoromethyl)phenyl]amino}carbonylamino)phenyl]thiopheno[2,3-d]pyrimidine-6-carboxamide (0.800 g, 80%) as a yellow solid. MS m/z calculated for (M+H)⁺ 519, found 519.

Step g: 5-amino-2-(methylsulfinyl)-4-[3-({[4 (trifluoromethyl)phenyl]amino}carbonylamino)phenyl]thiopheno[2,3-d]pyrimidine-6-carboxamide. To a solution of 5-amino-2-methylthio-4-[3-({[4-(trifluoromethyl)phenyl]amino}carbonylamino)phenyl]thiopheno[2,3-d]pyrimidine-6-carboxamide (0.650 g, 1.55 mmol) in THF (20 mL) was added 70-75% 3-chloroperoxybenzoic acid (0.348 g, 1.55 mmol). The reaction mixture was stirred at room temperature for 1 hour then diluted with EtOAc (200 mL) and saturated sodium bisulfite (50 mL). The organic layer was washed with saturated NaHCO₃ and brine, dried over Na₂SO₄, and concentrated to give 5-amino-2-(methylsulfinyl)-4-[3-({[4-(trifluoromethyl)phenyl]amino}carbonylamino)phenyl]thiopheno[2,3-d]pyrimidine-6-carboxamide (0.600 g, 89%) as a yellow solid. MS m/z calculated for (M+H)⁺ 535, found 535.

Step h: 5-amino-2-[(2-hydroxy-tert-butyl)amino]-4-[3-({[4-(trifluoromethyl)phenyl]amino}carbonylamino)phenyl]thiopheno[2,3-d]pyrimidine-6-carboxamide. 5-amino-2-(methylsulfinyl)-4-[3-({[4-(trifluoromethyl)phenyl]amino}carbonylamino)phenyl]thiopheno[2,3-d]pyrimidine-6-carboxamide (0.159 g, 0.28 mmol) and 2-amino-2-methyl-1-propanol (0.200 g, 2.2 mmol) were dissolved in DMF (5 mL) and heated to 90° C. in a microwave for 30 minutes The resulting mixture was diluted with EtOAc (50 mL), washed with brine, and dried over Na₂SO₄. Volatiles were removed on a rotary evaporator and the crude product was dissolved in DMSO (1 mL) and purified using reverse-phase preparative HPLC (10-80% acetonitrile, 0.1% TFA, 30 minutes). The appropriate fractions were combined and concentrated on a rotary evaporator. The residues were dissolved in ethyl acetate (50 mL), washed with aqueous NaHCO₃, and dried over Na₂SO₄. The solvent was removed in vacuo to give 5-amino-2-[(2-hydroxy-tert-butyl)amino]-4-[3-({[4-(trifluoromethyl)phenyl]amino}carbonylamino)phenyl]thiopheno[2,3-d]pyrimidine-6-carboxamide (65 mg, 40%) as a yellow solid. ¹H NMR (400 MHz, DMSO-D6) δ ppm 1.32 (s, 6H) 3.53 (d, J=5.08 Hz, 2H) 4.84 (t, J=5.86 Hz, 1H) 6.08 (s, 2H) 6.97 (s, 2H) 7.03 (s, 1H) 7.17 (d, J=7.03 Hz, 1H) 7.46 (t, J=8.00 Hz, 1H) 7.56-7.68 (m, 5H) 7.69 (s, 1H) 9.16 (s, 1H) 9.29 (s, 1H). MS m/z calculated for (M+H)⁺ 560, found 560. Analytical HPLC retention time: 5.72 minutes (method I).

### 5.2.133 2-[((1R)-1-ethyl-2-hydroxyethyl)amino]-5-amino-4-[3-({[4-(trifluoromethyl)phenyl]amino}carbonylamino)phenyl]thiopheno[2,3-d]pyrimidine-6-carboxamide

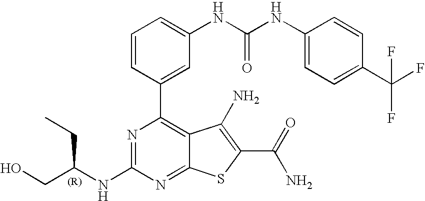

5-amino-2-(methylsulfinyl)-4-[3-({[4-(trifluoromethyl)phenyl]amino}carbonylamino)phenyl]thiopheno[2,3-d]pyrimidine-6-carboxamide (0.250 g, 0.28 mmol) and (R)-(−)-2amino-1-butanol (0.200 g, 2.2 mmol) were dissolved in DMF (5 mL) and heated to 90° C. in a microwave for 30 minutes The resulting mixture was diluted with EtOAc (50 mL), washed with brine, and dried over Na₂SO₄. Volatiles were removed on a rotary evaporator and the crude product was dissolved in DMSO (1 mL) and purified using reverse-phase preparative HPLC (10-80% acetonitrile, 0.1% TFA, 30 minutes). The appropriate fractions were combined and concentrated on a rotary evaporator. The residues were dissolved in ethyl acetate (50 mL), washed with aqueous NaHCO₃, and dried over Na₂SO₄. The solvent was removed in vacuo to give 2-[((1R)-1-ethyl-2-hydroxyethyl)amino]-5-amino-4-[3-({[4-(trifluoromethyl)phenyl]amino}carbonylamino)phenyl]thiopheno[2,3-d]pyrimidine-6-carboxamide (110 mg, 42%) as a yellow solid. ¹H NMR (400 MHz, DMSO-D6) δ ppm 0.82-0.98 (m, 3H) 1.38-1.53 (m, 1H) 1.62-1.77 (m, 1H) 3.36-3.43 (m, 1H) 3.49 (d, J=4.30 Hz, 1H) 3.92 (s, 1H) 4.67 (s, 1H) 5.99-6.20 (m, 2H) 6.98 (s, 2H) 7.20 (s, 1H) 7.48 (t, J=7.22 Hz, 2H) 7.55-7.70 (m, 5H) 7.74 (s, 1H) 9.07 (s, 1H) 9.19 (s, 1H). MS m/z calculated for (M+H)⁺ 560, found 560. Analytical HPLC retention time: 5.56 minutes (method I).

### 5.2.134 2-[((1S)-1-ethyl-2-hydroxyethyl)amino]-5-amino-4-[3-({[4-(trifluoromethyl)phenyl]amino}carbonylamino)phenyl]thiopheno[2,3-d]pyrimidine-6-carboxamide

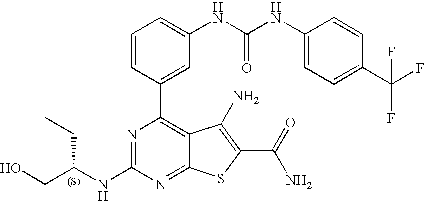

5-amino-2-(methylsulfinyl)-4-[3-({[4-(trifluoromethyl)phenyl]amino}carbonylamino)phenyl]thiopheno[2,3-d]pyrimidine-6-carboxamide (0.100 g, 0.28 mmol) and (S)-(−)-2-amino-1-butanol (0.100 g, 1.2 mmol) were dissolved in DMF (5 mL) and heated to 90° C. in a microwave for 30 minutes The resulting mixture was diluted with EtOAc (50 mL), washed with brine, and dried over Na₂SO₄. Volatiles were removed on a rotary evaporator and the crude product was dissolved in DMSO (1 mL) and purified using reverse-phase preparative HPLC (10-80% acetonitrile, 0.1% TFA, 30 minutes). The appropriate fractions were combined and concentrated on a rotary evaporator. The residues were dissolved in ethyl acetate (50 mL), washed with aqueous NaHCO₃, and dried over Na₂SO₄. The solvent was removed in vacuo to give 2-[((1S)-1-ethyl-2-hydroxyethyl)amino]-5-amino-4-[3-({[4-(trifluoromethyl)phenyl]amino}carbonylamino)phenyl]thiopheno[2,3-d]pyrimidine-6-carboxamide (50 mg, 42%) as a yellow solid. ¹H NMR (400 MHz, DMSO-D6) δ ppm 0.82-0.98 (m, 3H) 1.38-1.53 (m, 1H) 1.62-1.77 (m, 1H) 3.36-3.43 (m, 1H) 3.49 (d, J=4.30 Hz, 1H) 3.92 (s, 1H) 4.67 (s, 1H) 5.99-6.20 (m, 2H) 6.98 (s, 2H) 7.20 (s, 1H) 7.48 (t, J=7.22 Hz, 2H) 7.55-7.70 (m, 5H) 7.74 (s, 1H) 9.07 (s, 1H) 9.19 (s, 1H). MS m/z calculated for (M+H)⁺ 560, found 560. Analytical HPLC retention time: 5.56 minutes (method I).

### 5.2.135 2-[((1R)-2-hydroxy-isopropyl)amino]-5-amino-4-[3-({[4-(trifluoromethyl)phenyl]amino}carbonylamino)phenyl]thiopheno[2,3-d]pyrimidine-6-carboxamide

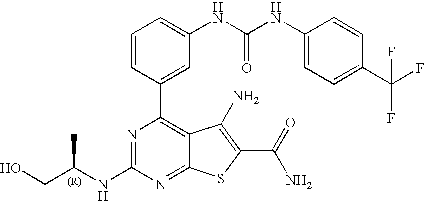

5-amino-2-(methylsulfinyl)-4-[3-({[4-(trifluoromethyl)phenyl]amino}carbonylamino)phenyl]thiopheno[2,3-d]pyrimidine-6-carboxamide (0.100 g, 0.18 mmol) and (R)-(−)-2amino-1-propanol (0.200 g, 2.2 mmol) were dissolved in DMF (5 mL) and heated to 90° C. in a microwave for 30 minutes The resulting mixture was diluted with EtOAc (50 mL), washed with brine, and dried over Na₂SO₄. Volatiles were removed on a rotary evaporator and the crude product was dissolved in DMSO (1 mL) and purified using reverse-phase preparative HPLC (10-80% acetonitrile, 0.1% TFA, 30 minutes). The appropriate fractions were combined and concentrated on a rotary evaporator. The residues were dissolved in ethyl acetate (50 mL), washed with aqueous NaHCO₃, and dried over Na₂SO₄. The solvent was removed in vacuo to give 50 mg (50%) as a yellow solid. ¹H NMR (400 MHz, DMSO-D6) δ ppm 1.15 (d, J=6.25 Hz, 3H) 3.31 (s, 2H) 3.41-3.57 (m, 1H) 4.64-4.78 (m, 1H) 6.07 (s, 2H) 6.98 (s, 2H) 7.17 (d, J=5.86 Hz, 1H) 7.47 (t, J=7.81 Hz, 2H) 7.58-7.72 (m, 5H) 7.75 (s, 1H) 9.55 (s, 1H) 9.67 (s, 1H). MS m/z calculated for (M+H)⁺ 546, found 546. Analytical HPLC retention time: 5.38 minutes (method I).

### 5.2.136 2-[((1S)-2-hydroxy-isopropyl)amino]-5-amino-4-[3-({[4-(trifluoromethyl)phenyl]amino}carbonylamino)phenyl]thiopheno[2,3-d]pyrimidine-6-carboxamide

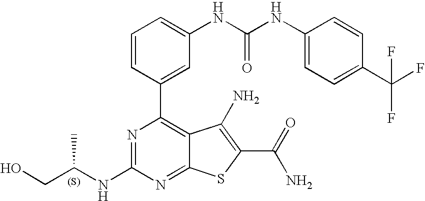

5-amino-2-(methylsulfinyl)-4-[3-({[4-(trifluoromethyl)phenyl]amino}carbonylamino) phenyl]thiopheno[2,3-d]pyrimidine-6-carboxamide (0.50 g, 0.09 mmol) and (R)-(−)-2-amino-1-propanol (0.100 g, 1.2 mmol) were dissolved in DMF (5 mL) and heated to 90° C. in a microwave for 30 minutes The resulting mixture was diluted with EtOAc (50 mL), washed with brine, and dried over Na₂SO₄. Volatiles were removed on a rotary evaporator and the crude product was dissolved in DMSO (1 mL) and purified using reverse-phase preparative HPLC (10-80% acetonitrile, 0.1% TFA, 30 minutes). The appropriate fractions were combined and concentrated on a rotary evaporator. The residues were dissolved in ethyl acetate (50 mL), washed with aqueous NaHCO₃, and dried over Na₂SO₄. The solvent was removed in vacuo to give 20 mg (48%) as a yellow solid. ¹H NMR (400 MHz, DMSO-D6) δ ppm 1.15 (d, J=6.25 Hz, 3H) 3.31 (s, 2H) 3.41-3.57 (m, 1H) 4.64-4.78 (m, 1H) 6.07 (s, 2H) 6.98 (s, 2H) 7.17 (d, J=5.86 Hz, 1H) 7.47 (t, J=7.81 Hz, 2H) 7.58-7.72 (m, 5H) 7.75 (s, 1H) 9.55 (s, 1H) 9.67 (s, 1H). MS m/z calculated for (M+H)⁺ 546, found 546. Analytical HPLC retention time: 5.37 minutes (method I).

### 5.2.137 5-amino-4-[3-({[4-(trifluoromethyl)phenyl]amino}carbonylamino)phenyl]thiopheno[2,3-d]pyrimidine-6-carboxamide

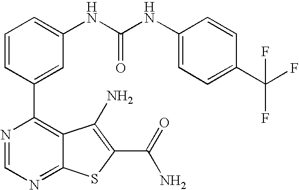

Step a: N-(3-(1,3-dioxolan-2-yl)phenyl){[4-(trifluoromethyl)phenyl]amino}carboxamide. 3-aminobenzaldehyde ethylene acetal (3.0 g, 18.5 mmol) and 4-(trifluoromethyl)phenyl isocyanate (4.0 g, 22. mmol) were dissolved in THF (20 mL) and heated at reflux with stirring under N₂ for 3 hours. The reaction mixture was cooled and diluted with hexane (100 mL) and the precipitate collected by filtration to give 4 g (60%) of N-(3-(1,3-dioxolan-2-yl)phenyl){[4-(trifluoromethyl)phenyl]amino}carboxamide as a white solid. MS m/z calculated for (M+H)⁺ 353, found 353.

Step b: N-(3-formylphenyl)([4-(trifluoromethyl)phenyl]amino)carboxamide. N-(3-(1,3-dioxolan-2-yl)phenyl) {[4-(trifluoromethyl)phenyl]amino}carboxamide (3.5 g, 9.9 mmol) was suspended in 80% aqueous HOAc and heated to 50° C. for 1 hour. The reaction mixture was cooled and diluted with ice water (200 mL) and the precipitate was collected by filtration and dried to give 2.5 g (83%) of N-(3-formylphenyl){[4-(trifluoromethyl)phenyl]amino}carboxamide as a white solid. ¹H NMR (400 MHz, DMSO-D6) δ ppm 7.47-7.60 (m, 2H) 7.60-7.77 (m, 6H) 8.11 (s, 1H) 8.31 (s, 1H) 9.99 (s, 1H). MS m/z calculated for (M+H)⁺ 309, found 309.

Step c: N-[3-(5-cyano-6-hydroxypyrimidin-4-yl)phenyl]{[4-(trifluoromethyl)phenyl]amino}carboxamide. N-(3-formylphenyl) {[4-(trifluoromethyl)phenyl]amino}carboxamide (2.5 g, 8.1 mmol), ethylcyanoacetate (0.90 g, 8.2 mmol) and piperidine (2 drops) were suspended in ethanol (40 mL) and heated to 75° C. for 1.5 hours. The reaction mixture was cooled to room temperature and formamidine (1.0 g, 10 mmol) and potassium carbonate (2.5 g, 32.5 mmol) were added. The resulting mixture was stirred vigorously and heated to 60° C. overnight. The reaction mixture was cooled in an ice bath and the resulting precipitate was collected by filtration, and dissolved in hot water. The resulting solution was cooled to 0° C. and acidified with 2 N HCl to pH 2. The resulting precipitate was collected by filtration, washed with ice water, and dried to give 2.0 g (62%) of N-[3-(5-cyano-6-hydroxypyrimidin-4-yl)phenyl]{[4-(trifluoromethyl)phenyl]amino}carboxamide as a white solid. ¹H NMR (400 MHz, DMSO-D6) δ ppm 7.50 (t, J=7.81 Hz, 1H) 7.53-7.60 (m, 1H) 7.60-7.73 (m, 5H) 8.10 (s, 1H) 8.58 (s, 1H) 9.11 (s, 1H) 9.18 (s, 1H). MS m/z calculated for (M+H)⁺ 400, found 400.

Step d: N-[3-(6-chloro-5-cyanopyrimidin-4-yl)phenyl]{[4-(trifluoromethyl)phenyl]amino}carboxamide. To N-[3-(5-cyano-6-hydroxypyrimidin-4-yl)phenyl]{[4-(trifluoromethyl)phenyl]amino}carboxamide (2.0 g, 5 mmol) in dioxane (30 mL) was added POCl₃ (15 mL). The resulting reaction mixture was heated at 80° C. under a reflux condenser under N₂ for 4 hours and then cooled to room temperature. The volatiles were removed on a rotary evaporator. The resulting residue was dissolved in 1,4-dioxane (10 ml) and ice water was added with stirring. The resulting precipitate was collected by filtration, washed with H₂O and dried. The solid was dissolved in EtAOc and silica gel was added and the volatiles removed on a rotary evaporator. Flash chromatography (30-50% EtOAc/hexane) gave 1.0 g (50%) of N-[3-(6-chloro-5-cyanopyrimidin-4-yl)phenyl]{[4-(trifluoromethyl)phenyl]amino}carboxamide as a white solid. MS m/z calculated for (M+H)⁺ 418, found 418.

Step e: N-{3-[6-(carbamoylmethylthio)-5-cyanopyrimidin-4-yl]phenyl}{[4-(trifluoromethyl)phenyl]amino}carboxamide. N-[3-(6-chloro-5-cyanopyrimidin-4-yl)phenyl]{[4-(trifluoromethyl)phenyl]amino}carboxamide (0.500 g, 1.2 mmol), 2-mercaptoacetamide (0.150 g, 1.4 mmol), K₂CO₃ (0.700 g, 2.4 mmol), and ethanol (20 mL) were combined in a flask, stirred vigorously, and heated at 50° C. under a reflux condenser under N₂ for 16 hours. The reaction mixture was cooled to room temperature and 100 mL of ice water was added. The resulting solids were collected by vacuum filtration, washed once with H₂O, twice with 30% Et₂O/hexane, and dried under high vacuum at 50° C. to give 481 mg (85%) of N-{3-[6-(carbamoylmethylthio)-5-cyanopyrimidin-4-yl]phenyl}{[4-(trifluoromethyl)phenyl]amino}carboxamide as a light yellow solid. MS m/z calculated for (M+H)⁺ 473, found 473.

Step f: 5-amino-4-[3-({[4-(trifluoromethyl)phenyl]amino}carbonylamino)phenyl]thiopheno[2,3-d]pyrimidine-6-carboxamide. A stirred suspension of N-{3-[6-(carbamoylmethylthio)-5-cyanopyrimidin-4-yl]phenyl}{[4-(trifluoromethyl)phenyl]amino}carboxamide (0.480 g, 1.0 mmol) in ethanol (20 mL) was cooled to 0° C. under N₂. Freshly prepared NaOEt (4 mmol) in ethanol (10 mL) was added and the resulting mixture stirred 2 hours while being slowly warmed to room temperature. The mixture was then heated at 45° C. for 2 hours. HCl (5 mmol) in 50 mL of ice water was added with vigorous stirring. The resulting solids were collected by vacuum filtration, washed once with H₂O, once with 20% Et₂O/hexane, and then dried under high vacuum. The solid was dissolved in DMSO (1 mL) and purified using reverse-phase preparative HPLC (10-80% acetonitrile, 0.1% TFA, 30 minutes). The appropriate fractions were combined and concentrated on a rotary evaporator. The residues were dissolved in ethyl acetate (50 mL), washed with aqueous NaHCO₃, and dried over Na₂SO₄. The solvent was removed in vacuo to give 100 mg (21%) of 5-amino-4-[3-({[4-(trifluoromethyl)phenyl]amino}carbonylamino)phenyl]thiopheno[2,3-d]pyrimidine-6-carboxamide. ¹H NMR (400 MHz, DMSO-D6) δ ppm 6.28 (s, 2H) 7.22-7.34 (m, 1H) 7.43 (s, 2H) 7.53 (t, J=7.81 Hz, 1H) 7.58-7.73 (m, 5H) 7.83 (t, J=1.76 Hz, 1H) 9.12 (s, 1H) 9.16 (s, 1H) 9.22 (s, 1H). MS m/z calculated for (M+H)⁺ 473, found 573. Analytical HPLC retention time: 5.48 minutes (method I).

### 5.2.138 5-amino-4-(3,4-dichloro-5-methoxyphenyl)-2-(1-hydroxy-2-methylpropan-2-ylamino)thieno[2,3-d]pyrimidine-6-carboxamide

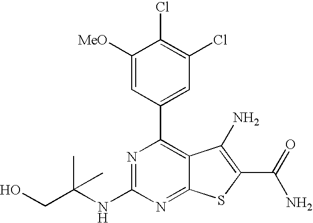

Step a: methyl 4-hydroxy-3-methoxy-5-nitrobenzoate: Methylvanilate (25.0 g, 137.3 mmol) was dissolved in CH₂Cl₂ (200 mL) with stirring under N₂. The resulting solution was cooled to −50° C. and fuming nitric acid (60 mL, 1373 mmol) was added dropwise. The resulting mixture was stirred for 2 hours at −50° C. then added to stirring ice water slowly. The resulting yellow precipitate was collected by filtration, washed with H₂O, and dried to give 14.0 g (47%) methyl 4-hydroxy-3-methoxy-5-nitrobenzoate. ¹H NMR (400 MHz, CHLOROFORM-D) δ ppm 3.97 (s, 3H) 4.04 (s, 3H) 7.90 (s, 1H) 8.043 (s, 1H) 11.14 (s, 1H).

Step b: methyl 4-chloro-3-methoxy-5-nitrobenzoate. Methyl 4-hydroxy-3-methoxy-5-nitrobenzoate (13.3 g, 58.6 mmol) was dissolved in DMF (100 mL) with stirring under N₂. The resulting solution was cooled to −20° C. and oxalyl chloride (16 mL, 175.7 mmol) was added dropwise. A heavy precipitate formed during addition. The ice/salt bath was removed and the temperature slowly raised to 80° C. and kept at 80° C. for 3 hours. The resulting mixture was cooled to room temperature, poured into ice water, and stirred for 15 minutes The resulting yellow precipitate was collected by filtration, washed with H₂O, and dried to give 12.0 g (85%) methyl 4-chloro-3-methoxy-5-nitrobenzoate. ¹H NMR (400 MHz, CHLOROFORM-D) δ ppm 3.97 (s, 3H) 4.04 (s, 3H) 7.77 (s, 1H) 8.03 (s, 1H).

Step c: methyl 5-amino-4-chloro-3-methoxybenzoate. A mixture of methyl 4-chloro-3-methoxy-5-nitrobenzoate (12.0 g, 48.97 mmol), EtOH (200 mL), water (200 mL) and iron powder (8.2 g, 146.93 mmol) was brought to reflux during which time a flow of N₂ was passed through the mixture to degas it from oxygen for 20 minutes The refluxing mixture was treated with HCl (0.5 mL) and stirred overnight. The reaction mixture was cooled and celite (2.0 g) was added, stirred for 10 minutes and filtered through a pad of Celite. The resulting mixture was concentrated on a rotary evaporator, extracted with EtOAc (200 mL), washed with brine, and dried over Na₂SO₄. The solvent was removed in vacuo to give 9.0 g (86%) of methyl 5-amino-4-chloro-3-methoxybenzoate as a yellow solid. ¹H NMR (300 MHz, CHLOROFORM-D) δ ppm 3.90 (s, 3H) 3.93 (s, 3H) 4.22 (s, 2H) 7.01 (s, 1H) 7.13 (s, 1H).

Step d: methyl 4,5-dichloro-3-methoxybenzoate. Anhydrous copper II chloride (6.8 g, 50.4 mmol), butylnitrite (6.5 g, 62.8 mmol) and anhydrous acetonitrile were added to a three-necked round-bottom flask that was equipped with a reflux condenser, addition funnel and a gas outlet tube. The resulting reaction mixture was warmed to 65° C. and methyl 5-amino-4-chloro-3-methoxybenzoate (9.0 g, 41.86 mmol) in acetonitrile was added dropwise. After the reaction mixture was cooled to room temperature, it was poured into 20% aqueous HCl (100 mL) and neutralized by addition of saturated sodium carbonate. The resulting mixture was extracted with CH₂Cl₂ (200 mL), washed with brine, and dried over Na₂SO₄. The solvent was removed in vacuo to give 7.2 g (97%) of methyl 4,5-dichloro-3-methoxybenzoate as a yellow solid.

Step e: 4,5-dichloro-3-methoxybenzoicacid. Methyl 4,5-dichloro-3-methoxybenzoate (7.0 g, 29.9 mmol) was suspended in NaOH (120 mL, 0.6 N) and heated at 100° C. under a reflux condenser till all ester was dissolved and one phase solution obtained. The resulting solution was cooled to 0° C. and acidified with 2 N HCl to pH 2. The resulting precipitate was collected by filtration, washed with water, crystallized from EtOH, and dried to give 6.0 g (92%) of 4,5-dichloro-3-methoxybenzoic acid as a yellow solid. ¹H NMR (400 MHz, CHLOROFORM-D) δ ppm 3.99 (s, 3H) 7.54 (s, 1H) 7.85 (s, 1H).

Step f: (4,5-dichloro-3-methoxyphenyl)methan-1-ol. 4,5-dichloro-3-methoxybenzoic acid (6.0 g, 27.3 mmol) was dissolved in dry THF (50 mL) with stirring under N₂. BH₃/THF (86 mL, 86 mmol) was added and the resulting mixture heated at 75° C. under reflux condenser under N₂ for 16 hours. The mixture was cooled to room temperature, diluted with EtOAc, shaken, and the layers separated. The organics were washed with H₂O, dried over MgSO₄, filtered, and concentrated on a rotary evaporator to give 5.6 g (100%) of (4,5-dichloro-3-methoxyphenyl)methan-1-ol as a yellow solid. ¹H NMR (400 MHz, CHLOROFORM-D) δ ppm 3.92 (s, 3H) 4.67 (s, 2H) 6.88 (s, 1H) 7.07 (s, 1H).

Step g: 4,5-dichloro-3-methoxybenzaldehyde. (4,5-dichloro-3-methoxyphenyl)methan-1-ol (5.6 g, 27.1 mmol) was dissolved in dry benzene (100 mL) with stirring under N₂. Activated MnO₂ (8.0 g, 89.2 mmol) was added and the resulting mixture heated at 85° C. under reflux condenser under N₂ for 16 hours. The reaction mixture was cooled and Celite (2.0 g) was added, stirred for 10 minutes, and filtered through a pad of Celite. The resulting mixture was concentrated on a rotary evaporator, diluted with EtOAc (200 mL), washed with brine, and dried over Na₂SO₄. Flash chromatography (50% EtOAc/hexane) gave 5.2 g (94.5% yield) of 4,5-dichloro-3-methoxybenzaldehyde as a yellow solid. ¹H NMR (400 MHz, CHLOROFORM-D) δ ppm 4.00 (s, 3H) 7.35 (s, 1H) 7.58 (s, 1H) 9.91 (s, 1H).

Step h: 6-(4,5-dichloro-3-methoxyphenyl)-4-hydroxy-2-methylthiopyrimidine-5-carbonitrile. 4,5-dichloro-3-methoxybenzaldehyde (5.2 g, 25.3 mmol), ethyl cyanoacetate (2.86 g, 25.3 mmol), and piperidine (2 drops) were refluxed in toluene (15 mL) with azeotropic removal of H₂O under N₂. After 1.5 hours the stirbar was removed and the volatiles removed on a rotary evaporator. To the resulting material was added sequentially: a stirbar, s-methylisothiouronium sulfate (3.5 g, 12.65 mmol), K₂CO₃ (9.0 g, 66 mmol), and ethanol (50 mL). The resulting mixture was stirred vigorously and heated at 75° C. under a reflux condenser under N₂ for 4 hours. The reaction mixture was cooled in an ice bath and the resulting precipitate collected by filtration, washed with absolute EtOH, and dissolved in hot water. The resulting solution was cooled to 0° C. and acidified with 2 N HCl to pH 2. The resulting precipitate was filtered, washed with ice water, and dried to give 3.0 g (35%) of 6-(4,5-dichloro-3-methoxyphenyl)-4-hydroxy-2-methylthiopyrimidine-5-carbonitrile as a white solid. MS m/z calculated for (M+H)⁺ 342, found 342.

Step i: 6-(4,5-dichloro-3-methoxyphenyl)-4-chloro-2-methylthiopyrimidine-5-carbonitrile. To 6-(4,5-dichloro-3-methoxyphenyl)-4-hydroxy-2-methylthiopyrimidine-5-carbonitrile (3.0 g, 8.8 mmol) in dioxane (60 mL) was added POCl₃ (30 mL). The resulting reaction mixture was heated at 85° C. under a reflux condenser under N₂ for 4 hours and then cooled to room temperature. The volatiles were removed on a rotary evaporator. The resulting residue was dissolved in 1,4-dioxane (20 ml) and ice water was added with stirring. The resulting precipitate was collected by filtration, washed with H₂O and dried. The solid was dissolved in EtOAc, silica gel was added, and the volatiles removed on a rotary evaporator. Flash chromatography (20% EtOAc/hexane) gave 2.0 g (64%) of 6-(4,5-dichloro-3-methoxyphenyl)-4-chloro-2-methylthiopyrimidine-5-carbonitrile as a white solid. MS m/z calculated for (M+H)⁺ 361, found 361.

Step j: 2-[6-(4,5-dichloro-3-methoxyphenyl)-5-cyano-2-methylthiopyrimidin-4-ylthio]acetamide. 6-(4,5-dichloro-3-methoxyphenyl)-4-chloro-2-methylthiopyrimidine-5-carbonitrile (2.0 g, 5.5 mmol), 2-mercaptoacetamide (0.7 g, 8.3 mmol), K₂CO₃ (1.5 g, 11.2 mmol), and ethanol (20 mL) were combined in a flask, stirred vigorously, and heated at 50° C. under a reflux condenser under N₂ for 16 hours. The reaction mixture was cooled to room temperature and 200 mL of ice water was added. The resulting solids were collected by vacuum filtration, washed once with H₂O, twice with 30% Et₂O/hexane, and dried under high vacuum at 50° C. to give 2.0 g (85%) of 2-[6-(4,5-dichloro-3-methoxyphenyl)-5-cyano-2-methylthiopyrimidin-4-ylthio]acetamide as a very light yellow solid. ¹H NMR (400 MHz, DMSO-D6) δ ppm 2.64 (s, 3H) 3.96 (s, 3H) 4.10 (s, 2H) 7.31 (s, 2H) 7.67 (s, 1H) 7.72 (s, 1H). MS m/z calculated for (M+H)⁺ 415, found 415.

Step k: 5-amino-4-(4,5-dichloro-3-methoxyphenyl)-2-methylthiothiopheno[2,3-d]pyrimidine-6-carboxamide. 2-[6-(4,5-dichloro-3-methoxyphenyl)-5-cyano-2-methylthiopyrimidin-4-ylthio]acetamide (2.0, 4.8 mmol) in ethanol (20 mL) was cooled to 0° C. under N₂. Freshly prepared NaOEt (4 mmol) in ethanol (10 mL) was added and the resulting mixture stirred 2 hours while being slowly warmed to room temperature. The mixture was then heated at 45° C. for 2 hours. HCl (5 mmol) in 50 mL of ice water was added with vigorous stirring. The resulting solids were collected by vacuum filtration, washed once with H₂O and then dried under high vacuum. The solid was dissolved in EtOAc, silica gel was added, and the volatiles removed on a rotary evaporator. Flash chromatography (50% EtOAc/hexane) gave 0.80 g (40%) of 5-amino-4-(4,5-dichloro-3-methoxyphenyl)-2-methylthiothiopheno[2,3-d]pyrimidine-6-carboxamide as a yellow solid. ¹H NMR (400 MHz, DMSO-D6) δ ppm 2.64 (s, 3H) 3.96 (s, 3H) 6.34 (s, 2H) 7.31 (s, 2H) 7.67 (s, 1H) 7.72 (s, 1H). MS m/z calculated for (M+H)⁺ 415, found 415.

Step 1: 5-amino-4-(4,5-dichloro-3-methoxyphenyl)-2-(methylsulfinyl)thiopheno[2,3-d]pyrimidine-6-carboxamide. To a solution of 5-amino-4-(4,5-dichloro-3-methoxyphenyl)-2-methylthiothiopheno[2,3-d]pyrimidine-6-carboxamide (0.800 g, 1.9 mmol) in THF (20 mL) was added 70-75% 3-chloroperoxybenzoic acid (0.692 g, 1.9 mmol). The reaction mixture was stirred at room temperature for 1 hour then diluted with EtOAc (200 mL) and saturated sodium bisulfite (50 mL). The organic layer was washed with saturated NaHCO₃ and brine, dried over Na₂SO₄, and concentrated to give 0.700 g (85%) of 5-amino-4-(4,5-dichloro-3-methoxyphenyl)-2-(methylsulfinyl)thiopheno[2,3-d]pyrimidine-6-carboxamide as a yellow solid. MS m/z calculated for (M+H)⁺ 431, found 431.

Step m: 5-amino-4-(4,5-dichloro-3-methoxyphenyl)-2-[(2-hydroxy-tert-butyl)amino]thiopheno[2,3-d]pyrimidine-6-carboxamide. 5-amino-4-(4,5-dichloro-3-methoxyphenyl)-2-(methylsulfinyl)thiopheno[2,3-d]pyrimidine-6-carboxamide (0.170 g, 0.39 mmol) and excess 2-amino-2-methyl-1-propanol (0.400 g, 4.4 mmol) were dissolved in DMF (3 mL) and heated to 90° C. in a microwave for 30 minutes The resulting mixture was diluted with EtOAc (50 mL), washed with brine, and dried over Na₂SO₄. Volatiles were removed on a rotary evaporator and the crude product was dissolved in DMSO (1 mL) and purified using reverse-phase preparative HPLC (10-80% acetonitrile, 0.1% TFA, 30 minutes). The appropriate fractions were combined and concentrated on a rotary evaporator. The residues were dissolved in ethyl acetate (50 mL), washed with aqueous NaHCO₃, and dried over Na₂SO₄. The solvent was removed in vacuo to give 0.085 g (48%) of 5-amino-4-(4,5-dichloro-3-methoxyphenyl)-2-[(2-hydroxy-tert-butyl)amino]thiopheno[2,3-d]pyrimidine-6-carboxamide as a yellow solid. MS m/z calculated for (M+H)⁺ 456, found 456.

### 5.2.139 5-amino-4-(4,5-dichloro-3-hydroxyphenyl)-2-[(2-hydroxy-tert-butyl)amino]thiopheno[2,3-d]pyrimidine-6-carboxamide

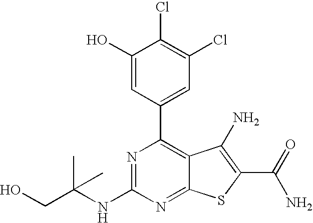

A solution of 5-amino-4-(4,5-dichloro-3-methoxyphenyl)-2-[(2-hydroxy-tert-butyl)amino]thiopheno[2,3-d]pyrimidine-6-carboxamide (0.080 g, 0.18 mmol) in CH₂Cl₂ (2 mL) was cooled to 0° C. and BBr₃ (1 M in CH₂Cl₂, 1 mL) was added dropwise. The reaction mixture was stirred at room temperature for 2 hours then diluted with EtOAc (50 mL) and washed with saturated NaHCO₃, brine, and dried over Na₂SO₄. Volatiles were removed on a rotary evaporator and the crude product was dissolved in DMSO (1 mL) and purified using reverse-phase preparative HPLC (10-80% acetonitrile, 0.1% TFA, 30 minutes). The appropriate fractions were combined and concentrated on a rotary evaporator. The residues were dissolved in ethyl acetate (20 mL), washed with aqueous NaHCO₃, and dried over Na₂SO₄. The solvent was removed in vacuo to give 0.035 g (46%) of 5-amino-4-(4,5-dichloro-3-hydroxyphenyl)-2-[(2-hydroxy-tert-butyl)amino]thiopheno[2,3-d]pyridine-6-carboxamide as a yellow solid. ¹H NMR (400 MHz, DMSO-D6) δ ppm. 1.33 (s, 6H) 3.48 (s, 1H) 3.54 (s, 2H) 6.24 (s, 2H) 7.01 (s, 2H) 7.12 (d, J=4.69 Hz, 2H) 7.26 (s, 1H) 11.09 (s, 1H). MS m/z calculated for (M+H)⁺ 442, found 442. Analytical HPLC retention time: 5.126 minutes (method I).

### 5.2.140 5-amino-4-(4-(benzyloxy)-2,3-dihydrobenzofuran-6-yl)thieno[2,3-d]pyrimidine-6-carboxamide

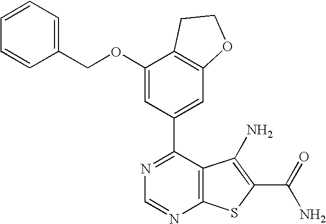

Step a: 2-(triphenylphosphoranylidene)succinic anhydride. A solution of maleic anhydride (20.0 g, 204 mmol) in acetone (100 mL) was added to a stirred solution of triphenylphosphine (53.5 g, 204 mmol) in acetone (200 mL) under N₂ in a flask cooled with a room temperature water bath. The resulting reaction mixture immediately became dark red in color. After about 10 minutes the reaction mixture became a solid mass. After 30 minutes the solids were collected by vacuum filtration and washed with acetone to give 50.54 g (68%) of 2-(triphenylphosphoranylidene)succinic anhydride as a light orange solid. ¹H NMR (300 MHz, DMSO-D6) δ ppm 3.27 (s, 2H) 7.57-7.80 (m, 15H). MS m/z calculated for (M+H)⁺ 361, found 361.

Step b: 2-(triphenylphosphoranylidene)butanedioic acid 1-ethyl ester. 2-(Triphenylphosphoranylidene)succinic anhydride (50.02 g, 138.8 mmol) was suspended in EtOH (460 mL) with stirring at room temperature under N₂. After stirring overnight, LCMS analysis of the reaction mixture indicated about 10% conversion to the desired product. The reaction mixture was heated at 40° C. under a reflux condenser under N₂ overnight. LCMS analysis of the resulting clear orange solution indicated complete conversion to the desired product. After cooling to room temperature the stirbar was removed and the volatiles removed on a rotary evaporator. The resulting solids were triturated with ethyl acetate and collected using vacuum filtration. The solids were washed with Et₂O and dried under high vacuum to give 43.51 g (77%) of 2-(triphenylphosphoranylidene)butanedioic acid 1-ethyl ester as a light orange solid. ¹H NMR (300 MHz, DMSO-D6) δ ppm 0.66 (t, J=7.00 Hz, 3H) 2.67 (d, J=17.31 Hz, 2H) 3.65 (q, J=6.96 Hz, 2H) 7.50-7.71 (m, 15H) 11.36 (s, 1H). MS m/z calculated for (M+H)⁺ 407, found 407.

Step c: 4,5-dihydrofuran-2-carbaldehyde. 2,3-dihydrofuran (27.30 g, 389 mmol) was dissolved with stirring in THF (600 mL) in a dry 2 L round bottom flask under N₂. The resulting solution was cooled with an acetone/dry ice bath and t-BuLi (250 mL of a 1.7 M solution in pentane, 425 mmol) was added slowly via addition funnel. The resulting mixture was stirred 20 minutes at about −70° C., 40 minutes at 0° C., and then recooled to about −70° C. (clear, green-yellow solution). DMF (50 mL, 646 mmol) was added dropwise via addition funnel and the resulting mixture stirred 1 hour while warming from −70° C. to 0° C. Water and saturated NH₄Cl were added and the resulting mixture extracted with Et₂O. The aqueous layer was acidified with 6 N HCl to about pH=6 and extracted 6 times with Et₂O. The aqueous layer was saturated with NaCl and extracted with Et₂O. The combined organics were dried over MgSO₄, filtered, and concentrated on a rotary evaporator. Flash chromatography over neutral alumina (50-80% Et₂O/hexane) gave 14.76 g (39%) of 4,5-dihydrofuran-2-carbaldehyde as a yellow oil. R_{f}=0.37 (50% EtOAc/hexane). ¹H NMR (300 MHz, DMSO-D6) δ ppm 2.83 (dt, J=9.82, 3.16 Hz, 2H) 4.41 (t, J=9.75 Hz, 2H) 6.38 (t, J=3.16 Hz, 1H) 9.42 (s, 1H). LCMS doesn't ionize.

Step d: (E)-4-(4,5-dihydrofuran-2-yl)-3-(ethoxycarbonyl)but-3-enoic acid. 2-(Triphenylphosphoranylidene)butanedioic acid 1-ethyl ester (43.45 g, 106.9 mmol) was added to a stirred solution of 4,5-dihydrofuran-2-carbaldehyde (11.01 g, 112.3 mmol) in benzene (275 mL) at room temperature under N₂. The resulting cloudy mixture was stirred 2 days at room temperature (slowly became clear). The stirbar was removed and the mixture concentrated on a rotary evaporator. The resulting residue was filtered through Celite and the filter cake washed with ethyl acetate. The filtrate was concentrated on a rotary evaporator. Flash chromatography (30-50-75% EtOAc/hexane) gave 28.45 g which was about 47% pure (˜55% yield) of (E)-4-(4,5-dihydrofuran-2-yl)-3-(ethoxycarbonyl)but-3-enoic acid as an orange oil. R_{f}=0.27 (50% EtOAc/hexane). ¹H NMR (300 MHz, DMSO-D6) δ ppm 1.21 (t, J=7.14 Hz, 3H) 2.67 (dt, J=9.48, 3.30 Hz, 2H) 3.62 (s, 2H) 4.13 (q, J=7.05 Hz, 2H) 4.38 (t, J=9.48 Hz, 2H) 5.66 (t, J=3.16 Hz, 1H) 7.05 (s, 1H) 12.25 (s, 1H). MS m/z calculated for (M+H)⁺ 227, found 227.

Step e: ethyl 4-hydroxy-2,3-dihydrobenzofuran-6-carboxylate. Oxalyl chloride (5.74 mL, 65.8 mmol) was added to a stirred solution of (E)-4-(4,5-dihydrofuran-2-yl)-3-(ethoxycarbonyl)but-3-enoic acid (10.6 g, 47.0 mmol) in CH₂Cl₂ (500 mL) at room temperature under N₂. The resulting mixture was heated at 41° C. under a reflux condenser under N₂ for 90 minutes and then poured onto water. Saturated NaHCO₃ was added and the resulting mixture shaken in a separatory funnel. The layers were separated and the organics washed with saturated NaHCO₃ and brine. The organics were dried over MgSO₄, filtered, and concentrated on a rotary evaporator. Flash chromatography (20-30-50% EtOAc/hexane) gave 5.76 g (59%) of ethyl 4-hydroxy-2,3-dihydrobenzofuran-6-carboxylate as a light yellow solid. R_{f}=0.42 (50% EtOAc/hexane). ¹H NMR (300 MHz, DMSO-D6) δ ppm 1.29 (t, J=7.14 Hz, 3H) 3.09 (t, J=8.79 Hz, 2H) 4.25 (q, J=7.14 Hz, 2H) 4.56 (t, J=8.79 Hz, 2H) 6.76 (d, J=1.37 Hz, 1H) 6.98 (d, J=1.37 Hz, 1H) 9.90 (s, 1H). MS m/z calculated for (M+H)⁺ 209, found 209.

Step f: ethyl 4-(benzyloxy)-2,3-dihydrobenzofuran-6-carboxylate. Ethyl 4-hydroxy-2,3-dihydrobenzofuran-6-carboxylate (5.76 g, 27.7 mmol) and tetrabutylammonium iodide (0.51 g, 1.38 mmol) were dissolved in THF (90 mL) under N₂ with stirring. Benzyl bromide (3.94 mL, 33.2 mmol) then NaH (0.80 g, 33.2 mmol) were added and the resulting mixture stirred at room temperature for 10 min. NMP (30 mL) was added and the resulting mixture heated at 40° C. under a reflux condenser under N₂ for 30 minutes. The reaction mixture was cooled to room temperature, quenched with saturated aqueous NH₄Cl, and extracted with EtOAc. The organics were washed with brine, concentrated on a rotary evaporator, and loaded onto a silica gel column with CH₂Cl₂. Flash chromatography (10-20% EtOAc/hexane) gave 6.64 g (80%) of ethyl 4-(benzyloxy)-2,3-dihydrobenzofuran-6-carboxylate as a white solid. R_{f}=0.44 (30% EtOAc/hexane). ¹H NMR (300 MHz, DMSO-D6) δ ppm 1.30 (t, J=7.00 Hz, 3H) 3.17 (t, J=8.79 Hz, 2H) 4.27 (q, J=6.96 Hz, 2H) 4.61 (t, J=8.79 Hz, 2H) 5.20 (s, 2H) 6.95 (d, J=1.10 Hz, 1H) 7.16 (d, J=1.10 Hz, 1H) 7.30-7.50 (m, 5H). MS m/z calculated for (M+H)⁺ 299, found 299.

Step g: (4-(benzyloxy)-2,3-dihydrobenzofuran-6-yl)methanol. LAH (0.80 g, 21.1 mmol) was weighed out in a dry 250 mL round-bottom flask with a stirbar. THF (20 mL) was added and the resulting mixture stirred at 0° C. under N₂. Ethyl 4-(benzyloxy)-2,3-dihydrobenzofuran-6-carboxylate (3.00 g, 10.1 mmol) in THF (23 mL) was added slowly via pipette. The resulting reaction mixture was stirred 2 hours while being slowly warmed from 0° C. to room temperature. Saturated aqueous Na₂SO₄ was added slowly until the reaction was quenched and the aluminum salts were white. The resulting mixture was filtered through Celite and the filter cake washed with EtOAc. The filtrate was concentrated on a rotary evaporator and dried under high vacuum to give 2.67 g (100%) of (4-(benzyloxy)-2,3-dihydrobenzofuran-6-yl)methanol as a nearly colorless oil which solidified upon standing. ¹H NMR (300 MHz, DMSO-D6) δ ppm 3.07 (t, J=8.65 Hz, 2H) 4.39 (d, J=5.77 Hz, 2H) 4.52 (t, J=8.79 Hz, 2H) 5.11 (s, 2H) 5.14 (t, J=5.77 Hz, 1H) 6.37 (s, 1H) 6.54 (s, 1H) 7.28-7.48 (m, 5H). MS m/z calculated for (M+H)⁺ 257, found 257.

Step h: 4-(benzyloxy)-2,3-dihydrobenzofuran-6-carbaldehyde. (4-(benzyloxy)-2,3-dihydrobenzofuran-6-yl)methanol (2.66 g, 10.4 mmol) was dissolved in CH₂Cl₂ (wet) (30 mL) with stirring. Dess-Martin periodinane (4.84 g, 11.4 mmol) was added and the resulting clear orange solution capped and stirred at room temperature for 3 hours. Saturated aqueous NaHCO₃ (20 mL) and saturated aqueous Na₂S₂O₃ (20 mL) were added and the resulting mixture shaken in a separatory funnel. The organics were washed with brine and concentrated on a rotary evaporator with silica gel. Flash chromatography (10-25% EtOAc/hexane) gave 2.38 g (90%) of 4-(benzyloxy)-2,3-dihydrobenzofuran-6-carbaldehyde as a nearly colorless oil. R_{f}=0.39 (30% EtOAc/hexane). ¹H NMR (300 MHz, DMSO-D6) δ ppm 3.20 (t, J=8.79 Hz, 2H) 4.63 (t, J=8.79 Hz, 2H) 5.24 (s, 2H) 6.95 (d, J=1.10 Hz, 1H) 7.18 (d, J=1.10 Hz, 1H) 7.30-7.51 (m, 5H) 9.86 (s, 1H). MS m/z calculated for (M+H)⁺ 255, found 255.

Step i: ethyl 3-(4-(benzyloxy)-2,3-dihydrobenzofuran-6-yl)-2-cyanoacrylate. 4-(benzyloxy)-2,3-dihydrobenzofuran-6-carbaldehyde (2.37 g, 9.32 mmol), ethyl cyanoacetate (1.05 g, 9.32 mmol), piperidine (2 drops), and toluene (15 mL) were combined in a 100 mL round-bottom flask, stirred vigorously, and heated at 130° C. with azeotropic removal of water for 1.5 hours. The stirbar was removed, all volatiles were removed on a rotary evaporator, and the residue dried under high vacuum to give 3.14 g (96%) of ethyl 3-(4-(benzyloxy)-2,3-dihydrobenzofuran-6-yl)-2-cyanoacrylate as a bright yellow solid. ¹H NMR (300 MHz, DMSO-D6) δ ppm 1.30 (t, J=7.14 Hz, 3H) 3.20 (t, J=8.79 Hz, 2H) 4.31 (q, J=7.05 Hz, 2H) 4.63 (t, J=8.79 Hz, 2H) 5.19 (s, 2H) 7.23 (s, 1H) 7.30-7.52 (m, 6H) 8.30 (s, 1H). MS m/z calculated for (M+H)⁺ 350, found 350.

Step j: 4-(4-(benzyloxy)-2,3-dihydrobenzofuran-6-yl)-6-hydroxypyrimidine-5-carbonitrile. Ethyl 3-(4-(benzyloxy)-2,3-dihydrobenzofuran-6-yl)-2-cyanoacrylate (3.14 g, 8.99 mmol), formamidine acetate (1.46 g, 14.0 mmol), K₂CO₃ (3.86 g, 28.0 mmol), and EtOH (15 mL) were combined in a 100 mL round-bottom flask, stirred vigorously, and heated at 80° C. under a reflux condenser under air for 15 hours. The resulting mixture was cooled to room temperature and water (20 mL) was added. The resulting mixture was extracted twice with EtOAc. The combined organics were washed with brine and concentrated on a rotary evaporator. The resulting residue was triturated with Et₂O and the solids collected using vacuum filtration. The solids were washed with Et₂O and dried under high vacuum to give 3.12 g of impure 4-(4-(benzyloxy)-2,3-dihydrobenzofuran-6-yl)-6-hydroxypyrimidine-5-carbonitrile which was used in the next reaction without further purification. ¹H NMR (300 MHz, DMSO-D6) δ ppm 3.15 (t, J=8.65 Hz, 2H) 4.59 (t, J=8.79 Hz, 2H) 5.15 (s, 2H) 6.81 (d, J=1.10 Hz, 1H) 7.02 (d, J=1.10 Hz, 1H) 7.30-7.50 (m, 5H) 8.19 (s, 1H). MS m/z calculated for (M+H)⁺ 346, found 346.

Step k: 4-(4-(benzyloxy)-2,3-dihydrobenzofuran-6-yl)-6-chloropyrimidine-5-carbonitrile. To a stirred solution of 4-(4-(benzyloxy)-2,3-dihydrobenzofuran-6-yl)-6-hydroxypyrimidine-5-carbonitrile (3.11 g, 9.01 mmol) in 1,4-dioxane (25 mL) was added POCl₃ (1.65 mL, 18.0 mmol). The resulting reaction mixture was heated at 90° C. with stirring under a reflux condenser under N₂ for 4 hours. The resulting reaction mixture was cooled to room temperature and ice was added to quench. The mixture was poured onto saturated aqueous NaHCO₃ and extracted with EtOAc. The organics were washed with brine and concentrated on a rotary evaporator with silica gel. Flash chromatography (30-50% EtOAc) gave a solid which was triturated with Et₂O and dried under high vacuum to give 0.627 g, (19% over two steps) of 4-(4-(benzyloxy)-2,3-dihydrobenzofuran-6-yl)-6-chloropyrimidine-5-carbonitrile as a light yellow solid. R_{f}=0.33 (30% EtOAc/hexane). ¹H NMR (300 MHz, DMSO-D6) δ ppm 3.23 (t, J=8.79 Hz, 2H) 4.66 (t, J=8.65 Hz, 2H) 5.20 (s, 2H) 7.06 (d, J=1.37 Hz, 1H) 7.24 (d, J=1.10 Hz, 1H) 7.30-7.55 (m, 5H) 9.29 (s, 1H). MS m/z calculated for (M+H)⁺ 364, found 364.

Step 1: 5-amino-4-(4-(benzyloxy)-2,3-dihydrobenzofuran-6-yl)thieno[2,3-d]pyrimidine-6-carboxamide. 4-(4-(benzyloxy)-2,3-dihydrobenzofuran-6-yl)-6-chloropyrimidine-5-carbonitrile (623 mg, 1.71 mmol), 2-mercaptoacetamide (164 mg, 1.80 mmol), K₂CO₃ (249 mg, 1.80 mmol), and EtOH (16 mL) were combined in a 100 mL round-bottom flask, stirred vigorously, and heated at 50° C. under a reflux condenser under N₂ for 2 hours. 2-mercaptoacetamide (20 mg, 0.22 mmol) was added and heating continued overnight. Ice water (35 mL) was added and the resulting mixture extracted with EtOAc. The organics were washed with brine and concentrated on a rotary evaporator. The residue was transferred to a 100 mL round-bottom flask with 20% MeOH/CH₂Cl₂, concentrated on a rotary evaporator, and dried under high vacuum to give impure 2-(6-(4-(benzyloxy)-2,3-dihydrobenzofuran-6-yl)-5-cyanopyrimidin-4-ylthio)acetamide as a brown solid. MS m/z calculated for (M+H)⁺ 419, found 419. Sodium (197 mg, 8.56 mmol) was added to dry EtOH (5 mL) and the resulting mixture stirred until all of the solid had dissolved. The resulting NaOEt solution was added to a stirred mixture of the crude 2-(6-(4-(benzyloxy)-2,3-dihydrobenzofuran-6-yl)-5-cyanopyrimidin-4-ylthio)acetamide in EtOH (20 mL) at 0° C. The resulting mixture was allowed to slowly warm to room temperature and stirred for 6 hours. The stirbar was removed and most of the solvent removed on a rotary evaporator. DMSO was added to dissolve the solids and the resulting solution filtered through a syringe filter. Trifluoroacetic acid (0.7 mL, 8.56 mmol) was added and the resulting solution purified using reverse phase preparatory HPLC (20-100% CH₃CN/H₂O). CH₃CN was removed on a rotary evaporator. The resulting aqueous mixture was extracted with EtOAc and the organics concentrated on a rotary evaporator nearly to dryness. DMSO and MeOH were added followed by EtOAc. The resulting solids were collected using vacuum filtration, washed with EtOAc and Et₂O, and dried under high vacuum to give 136 mg (19%) of 5-amino-4-(4-(benzyloxy)-2,3-dihydrobenzofuran-6-yl)thieno[2,3-d]pyrimidine-6-carboxamide as a bright yellow solid. MP=239° C. ¹H NMR (400 MHz, DMSO-D6) δ ppm 3.23 (t, J=8.79 Hz, 2H) 4.66 (t, J=8.59 Hz, 2H) 5.19 (s, 2H) 6.21 (s, 2H) 6.69 (s, 1H) 6.86 (s, 1H) 7.27-7.51 (m, 7H) 9.11 (s, 1H). MS m/z calculated for (M+H)⁺ 419, found 419.

### 5.2.141 5-amino-4-(4-hydroxy-2,3-dihydrobenzofuran-6-yl)thieno[2,3-d]pyrimidine-6-carboxamide

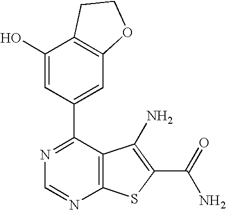

To a stirred solution of 5-amino-4-(4-(benzyloxy)-2,3-dihydrobenzofuran-6-yl)thieno[2,3-d]pyrimidine-6-carboxamide (85 mg, 0.20 mmol) in trifluoroacetic acid (3 mL) was added thioanisole (50 μL). The resulting red solution was stirred at room temperature overnight. More thioanisole (50 μL) was added and the resulting mixture stirred and heated at 45° C. for 6 hours and then at 55° C. for 12 hours. The stirbar was removed and the volatiles removed on a rotary evaporator. The resulting residue was dissolved in EtOAc (250 mL) and washed with saturated aqueous NaHCO₃ and brine. The organics were dried over MgSO₄, filtered, and concentrated on a rotary evaporator. The resulting residue was dissolved in DMSO/MeOH and purified using reverse phase preparatory HPLC (20-70% CH₃CN/H₂O). CH₃CN was removed on a rotary evaporator. The resulting aqueous mixture was extracted with EtOAc and the organics washed with saturated aqueous NaHCO₃ and brine, dried over MgSO₄, filtered, and concentrated on a rotary evaporator. A small amount of 20% MeOH/CH₂Cl₂ was added and the solids collected using vacuum filtration. The solids were washed with Et₂O and dried under high vacuum to give 32 mg (52%) of 5-amino-4-(4-hydroxy-2,3-dihydrobenzofuran-6-yl)thieno[2,3-d]pyrimidine-6-carboxamide as a bright yellow solid. MP 285° C. ¹H NMR (300 MHz, DMSO-D6) δ ppm 3.15 (t, J=8.65 Hz, 2H) 4.61 (t, J=8.65 Hz, 2H) 6.32 (s, 2H) 6.49 (d, J=1.37 Hz, 1H) 6.53 (d, J=1.37 Hz, 1H) 7.38 (s, 2H) 9.09 (s, 1H) 9.99 (s, 1H). MS m/z calculated for (M+H)⁺ 329, found 329.

### 5.2.142 5-amino-4-(3, 4-dichlorophenyl)thiopheno[2,3-d]pyrimidine-2,6-dicarboxamide

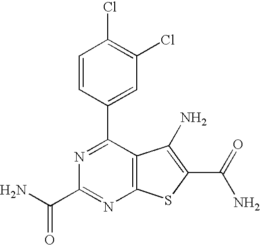

5-amino-4-(3,4-dichlorophenyl)thiopheno[2,3-d]pyrimidine-2,6-dicarboxamide. Suspended 5-amino-4-(3,4-dichlorophenyl)-2-cyanothiopheno[2,3-d]pyrimidine-6-carboxamide (850 mg, 2.33 mmol) in ethanol (50 mL), added 2.0 N NaOH (2.38 mL) and H₂O₂ (0.85 mL) and allowed to stir at room temp for 1.5 hours. Filtered precipitate and washed with ethanol. Purified using prep HPLC (20-100% MeCN/H₂O 30 min.) to give 15 mg as an orange solid. ¹H NMR (400 MHz, D6-DMSO) δ ppm 6.41 (s, 2H) 7.53 (s, 2H) 7.74 (d, J=8.1 Hz, 1H) 7.84 (d, J=8.1 Hz, 1H) 7.92 (s, 1H) 8.10 (s, 1H) 8.40 (s, 1H). MS m/z calculated for (M+H)⁺ 383, found 383. Analytical HPLC retention time: 4.58 min (method I).

### 5.2.143 5-amino-4-(3,4-dichlorophenyl)thiopheno[2,3-d]pyrimidine-6-carboxamide

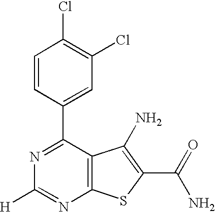

Step a: 6-(3,4-dichlorophenyl)-4-hydroxypyrimidine-5-carbonitrile. Placed 3,4-dichlorobenzaldehyde (8.0 g, 45.7 mmol), ethylcyanoacetate (5.4 mL, 50.3 mmol), formamidine (6.2 g, 59.4 mmol), potassium carbonate (18.9 g, 137.1 mmol), and ethanol (160 mL) in a round-bottomed flask and heated to 80° C. overnight. Filtered precipitate and washed with ethanol. Suspended the precipitate in water and filtered. Dried via toluene azeotrope to give 6.25 g as a white solid. ¹H NMR (400 MHz, D6-DMSO) δ ppm 7.45 (d, J=8.4 Hz, 1H) 7.79 (dd, J=2.0, 8.4 Hz, 1H) 7.99 (d, J=2.0 Hz, 1H) 8.24 (s, 1H). MS m/z calculated for (M+H)⁺ 266, found 266.

Step b: 6-(3,4-dichlorophenyl)-4-chloropyrimidine-5-carbonitrile. Dissolved 6-(3,4-dichlorophenyl)-4-hydroxypyrimidine-5-carbonitrile (6.25 g, 23.4 mmol) in anhydrous 1,4-dioxane. Added POCl₃ (25 mL) and heated to 100° C. overnight. Poured reaction into ice, neutralized with potassium carbonate, extracted with ethyl acetate (2×250 mL), dried with MgSO₄, and concentrated in vacuo to give 4.71 g as a white solid. ¹H NMR (400 MHz, D6-DMSO) δ ppm 7.95 (d, J=8.4 Hz, 1H) 7.99 (dd, J=2.0, 8.4 Hz, 1H) 8.22 (d, J=2.0 Hz, 1H) 9.36 (s, 1H). MS m/z calculated for (M+H)⁺ 285, found 285.

Step c: 2-[6-(3,4-dichlorophenyl)-5-cyanopyrimidin-4-ylthio]acetamide. Dissolved 6-(3,4-dichlorophenyl)-4-chloropyrimidine-5-carbonitrile (4.51 g, 15.8 mmol), 2-sulfanylacetamide (1.44 g, 15.8 mmol), and diisopropylethyl amine (4.1 mL, 23.7 mmol) in dichloromethane (40 mL) and ethanol (40 mL) and allowed to stir at room temperature for 1.5 hours. Filtered precipitate and washed with cold ethanol to give 2.72 g as a white solid. ¹H NMR (400 MHz, D6-DMSO) δ ppm 4.11 (s, 2H) 7.29 (s, 1H) 7.71 (s, 1H) 7.91 (d, J=8.4 Hz, 1H) 7.95 (dd, J=2.0, 8.4 Hz, 1H) 8.20 (d, J=2.0 Hz, 1H) 9.19 (s, 1H). MS m/z calculated for (M+H)⁺ 339, found 339.

Step d: 5-amino-4-(3,4-dichlorophenyl)thiopheno[2,3-d]pyrimidine-6-carboxamide. Prepared a sodium ethoxide solution (203 mg Na, 10 mL EtOH) added 2-[6-(3,4-dichlorophenyl)-5-cyanopyrimidin-4-ylthio]acetamide (1.0 g, 2.95 mmol) and allowed to stir at room temperature for 2.5 hours. Filtered precipitate and washed with cold ethanol. Purified 295 mg using prep HPLC (20-100% MeCN/H₂O 30 min.) to give 63 mg as a yellow solid. ¹H NMR (400 MHz, D6-DMSO) δ ppm 6.34 (s, 2H) 7.44 (s, 2H) 7.68 (dd, J=2.4, 8.4 Hz, 1H) 7.84 (d, J=8.4 Hz, 1H), 7.95 (d, J=2.4 Hz, 1H) 9.15 (s, 1H). MS m/z calculated for (M+H)⁺ 340, found 340. Analytical HPLC retention time: 5.28 min (method I).

### 5.2.144 5-amino-4-(3,4-dichlorophenyl)-2-methylthiopheno[2,3-d]pyrimidine-6-carboxamide

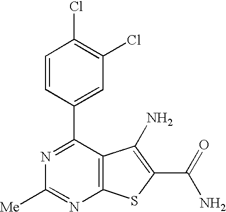

Step a: 6-(3,4-dichlorophenyl)-4-hydroxy-2-methylpyrimidine-5-carbonitrile. Placed 3,4-dichlorobenzaldehyde (8.0 g, 45.7 mmol), ethylcyanoacetate (5.4 mL, 50.3 mmol), acetamidine (5.6 g, 59.4 mmol), potassium carbonate (18.9 g, 137.1 mmol), and ethanol (160 mL) in a round-bottomed flask and heated to 80° C. overnight. Filtered precipitate and washed with ethanol. Suspended the precipitate in water and filtered. Dried via toluene azeotrope to give 10.2 g as a white solid. ¹H NMR (400 MHz, D6-DMSO) δ ppm 2.21 (s, 3H) 7.73 (d, J=8.4 Hz, 1H) 7.78 (dd, J=2.0, 8.4 Hz, 1H) 7.95 (d, J=2.0 Hz, 1H). MS m/z calculated for (M+H)⁺ 280, found 280.

Step b: 6-(3,4-dichlorophenyl)-4-chloro-2-methylpyrimidine-5-carbonitrile. Dissolved 6-(3,4-dichlorophenyl)-4-hydroxy-2-methylpyrimidine-5-carbonitrile (6.15 g, 22.0 mmol) in anhydrous 1,4-dioxane. Added POCl₃ (25 mL) and heated to 100° C. overnight. Poured reaction into ice, neutralized with potassium carbonate, extracted with ethyl acetate (2×250 mL), dried with MgSO₄, and concentrated in vacuo to give 4.71 g as a white solid. ¹H NMR (400 MHz, D6-DMSO) δ ppm 2.78 (s, 3H) 7.93 (d, J=8.2 Hz, 1H) 7.96 (dd, J=2.2, 8.2 Hz, 1H) 8.20 (d, J=2.2 Hz, 1H). MS m/z calculated for (M+H)⁺ 298, found 298.

Step c: 2-[6-(3,4-dichlorophenyl)-5-cyano-2-methylpyrimidin-4-ylthio]acetamide. Dissolved 6-(3,4-dichlorophenyl)-4-chloro-2-methylpyrimidine-5-carbonitrile (3.98 g, 13.3 mmol), 2-sulfanylacetamide (1.21 g, 13.3 mmol), and diisopropylethyl amine (3.5 mL, 20.0 mmol) in dichloromethane (40 mL) and ethanol (40 mL) and allowed to stir at room temperature for 1.5 hours. Filtered precipitate and washed with cold ethanol to give 2.57 g as a white solid. ¹H NMR (400 MHz, D6-DMSO) δ ppm 2.70 (s, 3H) 4.08 (s, 2H) 7.27 (s, 2H) 7.71 (s, 2H) 7.89 (d, J=8.4 Hz, 1H) 7.92 (dd, J=2.0, 8.4 Hz, 1H) 8.16 (d, J=2.0 Hz, 1H). MS m/z calculated for (M+H)⁺ 353, found 353.

Step d: 5-amino-4-(3,4-dichlorophenyl)-2-methylthiopheno[2,3-d]pyrimidine-6-carboxamide. Prepared a sodium ethoxide solution (195 mg Na, 10 mL EtOH) added 2-[6-(3,4-dichlorophenyl)-5-cyano-2-methylpyrimidin-4-ylthio]acetamide (1.0 g, 2.83 mmol) and allowed to stir at room temperature for 2.5 hours. Filtered precipitate and washed with cold ethanol. Purified 500 mg using prep HPLC (20-100% MeCN/H₂O 30 min.) to give 134 mg as a yellow solid. ¹H NMR (400 MHz, D6-DMSO) δ ppm 2.73 (s, 3H) 6.30 (s, 2H) 7.36 (s, 2H) 7.64 (dd, J=2.4, 8.4 Hz, 1H) 7.82 (d, J=8.4 Hz, 1H) 7.92 (d, J=2.4 Hz, 1H). MS m/z calculated for (M+H)⁺ 353, found 353. Analytical HPLC retention time: 5.44 minutes (method I).

### 5.2.145 5-amino-4-(3,4-dichlorophenyl)-2-(2-methylpropyl)thiopheno[2,3-d]pyrimidine-6-carboxamide

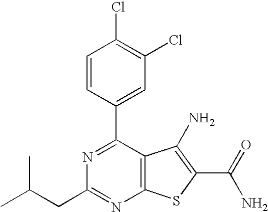

Step a: ethyl (2E)-3-(3,4-dichlorophenyl)-2-cyanoprop-2-enoate. Dissolved 3,4-dichlorobenzaldehyde (4.5 g, 25.7 mmol), ethylcyanoacetate (2.75 mL, 25.7 mmol), and piperidine (2 drops) in toluene. Heated to 135° C. in a Dean-Stark trap to drive off water. Removed solvent in vacuo to give 5.4 g as a yellow solid. ¹H NMR (400 MHz, D6-DMSO) δ ppm 1.31 (t, 3H) 4.33 (q, 2H) 7.90 (d, J=8.4 Hz, 1H) 8.06 (dd, J=2.4, 8.4 Hz, 1H) 8.30 (d, J=2.4 Hz, 1H) 8.43 (s, 1H).

Step b: 6-(3,4-dichlorophenyl)-4-hydroxy-2-(2-methylpropyl)pyrimidine-5-carbonitrile. Placed ethyl (2E)-3-(3,4-dichlorophenyl)-2-cyanoprop-2-enoate (2.0 g, 7.40 mmol), 3-methylbutanamidine (1.48 g, 14.8 mmol), potassium carbonate (3.06 g, 22.2 mmol), and ethanol (50 mL) in a round-bottomed flask and heated to 80° C. overnight. Filtered precipitate and washed with ethanol. Suspended the precipitate in water and filtered. Dried via toluene azeotrope to give 1.50 g as a white solid. MS m/z calculated for (M+H)⁺ 322, found 322.

Step c: 6-(3,4-dichlorophenyl)-4-chloro-2-(2-methylpropyl)pyrimidine-5-carbonitrile. Dissolved 6-(3,4-dichlorophenyl)-4-hydroxy-2-(2-methylpropyl)pyrimidine-5-carbonitrile (1.50 g, 7.40 mmol) in anhydrous 1,4-dioxane. Added POCl₃ (8 mL) and heated to 100° C. overnight. Poured reaction into ice, neutralized with potassium carbonate, extracted with ethyl acetate (2×150 mL), dried with MgSO₄, and concentrated in vacuo to give 733 mg as a yellow solid. MS m/z calculated for (M+H)⁺ 340, found 340.

Step d: 2-[6-(3,4-dichlorophenyl)-5-cyano-2-(2-methylpropyl)pyrimidin-4-ylthio]acetamide. Dissolved 6-(3,4-dichlorophenyl)-4-chloro-2-(2-methylpropyl)pyrimidine-5-carbonitrile (0.73 g, 2.16 mmol), 2-sulfanylacetamide (196 mg, 2.16 mmol), and diisopropylethyl amine (0.6 mL, 3.24 mmol) in dichloromethane (8 mL) and ethanol (8 mL) and allowed to stir at room temperature for 1.5 hours. Filtered precipitate and washed with cold ethanol to give 570 mg as a white solid. MS m/z calculated for (M+H)⁺ 395, found 395.

Step e: 5-amino-4-(3,4-dichlorophenyl)-2-(2-methylpropyl)thiopheno[2,3-d]pyrimidine-6-carboxamide. Prepared a sodium ethoxide solution (100 mg Na, 10 mL EtOH) added 2-[6-(3,4-dichlorophenyl)-5-cyano-2-(2-methylpropyl)pyrimidin-4-ylthio]acetamide (0.57 g, 1.44 mmol) and allowed to stir at room temperature for 2.5 hours. Filtered precipitate and washed with cold ethanol. Purified using prep HPLC (20-100% MeCN/H₂O 30 min.) to give 220 mg as a yellow solid. ¹H NMR (400 MHz, D6-DMSO) δ ppm MS m/z calculated for (M+H)⁺ 395, found 395. Analytical HPLC retention time: 6.56 minutes (method I).

### 5.2.146 2-{[(1S)-2-hydroxy-1-(methylethyl)ethyl]amino}-5-amino-4-(3,4-dichlorophenyl)thiopheno[2,3-d]pyrimidine-6-carboxamide

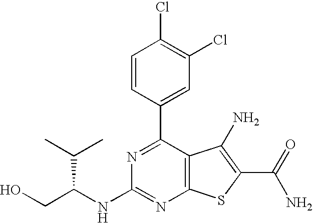

2-{[(1S)-2-hydroxy-1-(methylethyl)ethyl]amino}-5-amino-4-(3,4-dichlorophenyl)thiopheno[2,3-d]pyrimidine-6-carboxamide. 5-amino-4-(3,4-dichlorophenyl)-2-(methylsulfinyl)thiopheno[2,3-d]pyrimidine-6-carboxamide (360 mg, 0.90 mmol), (2S)-2-amino-3-methylbutan-1-ol (464 mg, 4.50 mmol), and anhydrous THF (15 mL) were placed in a round-bottomed flask and heated to 75° C. overnight. The volatiles were removed in vacuo, the residue suspended in ethanol, and the precipitate collected using vacuum filtration to give 170 mg as a yellow solid. Purified using prep HPLC (20-100% MeCN/H₂O 30 min.) to give 60 mg as a yellow solid. ¹H NMR (400 MHz, D6-DMSO) δ ppm 0.91 (s, 6H) 1.96 (m, 1H) 1.99 (s, 3H) 3.50 (m, 2H) 4.55 (m, 2H) 6.14 (s, 2H) 7.00 (s, 2H) 7.50 (m, 2H) 7.80 (m, 2H). MS m/z calculated for (M+H)⁺ 440, found 440. Analytical HPLC retention time: 5.60 min (method I).

### 5.2.147 2-{[(1R)-2-hydroxy-1-(methylethyl)ethyl]amino}-5-amino-4-(3,4-dichlorophenyl)thiopheno[2,3-d]pyrimidine-6-carboxamide

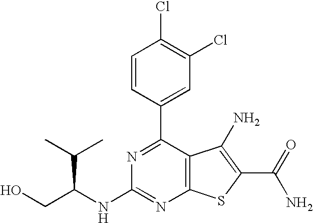

2-{[(1R)-2-hydroxy-1-(methylethyl)ethyl]amino}-5-amino-4-(3,4-dichlorophenyl)thiopheno[2,3-d]pyrimidine-6-carboxamide. 5-amino-4-(3,4-dichlorophenyl)-2-(methylsulfinyl)thiopheno[2,3-d]pyrimidine-6-carboxamide (360 mg, 0.90 mmol), (2S)-2-amino-3-methylbutan-1-ol (464 mg, 4.50 mmol), and anhydrous THF (15 mL) were placed in a round-bottomed flask and heated to 75° C. overnight. The volatiles were removed in vacuo, the residue suspended in ethanol, and the precipitate collected via vacuum filtration to give 150 mg as a yellow solid. Purified using prep HPLC (20-100% MeCN/H₂O 30 min.) to give 100 mg as a yellow solid. ¹H NMR (400 MHz, D6-DMSO) δ ppm 0.91 (s, 6H) 1.96 (m, 1H) 1.99 (s, 3H) 3.50 (m, 2H) 4.55 (m, 2H) 6.14 (s, 2H) 7.00 (s, 2H) 7.50 (m, 2H) 7.80 (m, 2H). MS m/z calculated for (M+H)⁺ 440, found 440. Analytical HPLC retention time: 5.60 min (method I).

### 5.2.148 5-amino-2-[(2-hydroxy-tert-butyl)amino]-4-(3-hydroxy-4-methylphenyl)thiopheno[2,3-d]pyrimidine-6-carboxamide

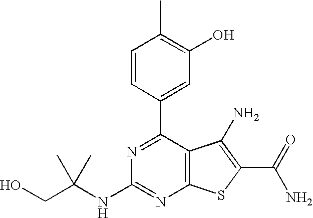

5-amino-2-[(2-hydroxy-tert-butyl)amino]-4-(3-hydroxy-4-methylphenyl)thiopheno[2,3-d]pyrimidine-6-carboxamide. Dissolved 5-amino-2-[(2-hydroxy-tert-butyl)amino]-4-(3-methoxy-4-methylphenyl)thiopheno[2,3-d]pyrimidine-6-carboxamide (110 mg, 0.27 mmol) in dichloromethane (4 mL) and added 1.0 M BBr₃ (1.08 mL, 1.08 mmol) and allowed to stir at room temp for 4 hours. Poured into water and separated layers. Adjusted pH=6, and extracted with ethyl acetate (3×50 mL), washed with brine (50 mL), dried with sodium sulfate. Purified using prep HPLC (20-80% MeCN/H₂O 30 min.) to give 30 mg as a yellow solid. ¹H NMR (400 MHz, D6-DMSO) δ ppm 1.33 (s, 6H) 2.20 (s, 3H) 3.54 (d, J=6.0 Hz, 2H) 4.86 (t, J=6.0 Hz, 1H) 6.10 (s, 2H) 6.95 (m, 5H) 7.23 (d, J=8.0 Hz, 1H) 9.75 (s, 1H). MS m/z calculated for (M+H)⁺ 388, found 388. Analytical HPLC retention time: 4.62 minutes (method I).

### 5.2.149 5-amino-4-(5-methoxy(3-pyridyl))thiopheno[2,3-d]pyrimidine-6-carboxamide

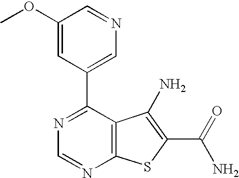

Step a: 4-hydroxy-6-(5-methoxy(3-pyridyl))pyrimidine-5-carbonitrile. 3-Methoxy-5-formylpyridine (2.00 g, 14.6 mmol), ethylcyanoacetate (1.67 g, 14.8 mmol), formamidine acetate (1.65 g, 14.6 mmol), and potassium carbonate (4.04 g, 29.2 mmol) were stirred in ethanol (80 mL) at 78° C. for 3 days. The solvent was removed in vacuo and 2 N HCl was added until the solution was pH 4. The product was collected by filtration, washed with water and ethanol and dried under vacuum to give 4-hydroxy-6-(5-methoxy(3-pyridyl))pyrimidine-5-carbonitrile (825 mg, 25%). MS m/z calculated for (M+H)⁺ 229, found 229.

Step b: 4-chloro-6-(5-methoxy(3-pyridyl))pyrimidine-5-carbonitrile. 4-hydroxy-6-(5-methoxy(3-pyridyl))pyrimidine-5-carbonitrile (820 mg, 3.6 mmol) was stirred in POCl₃ (20 mL) and the mixture was heated to 90° C. for 4 hours. The volatiles were removed under reduced pressure and the residue was partitioned between ethyl acetate and saturated NaHCO₃. The organic layer was separated and concentrated to give a residue that was purified using flash chromatography (100% EtOAc) to give 4-chloro-6-(5-methoxy(3-pyridyl))pyrimidine-5-carbonitrile (825 mg, 93%). MS m/z calculated for (M+H)⁺ 247, found 247.

Step c: 5-amino-4-(5-methoxy(3-pyridyl))thiopheno[2,3-d]pyrimidine-6-carboxamide. 4-chloro-6-(5-methoxy(3-pyridyl))pyrimidine-5-carbonitrile (0.88 g, 3.6 mmol), 2-mercaptoacetamide (0.341 g, 3.75 mmol), and sodium carbonate (0.453 g, 4.27 mmol) were stirred in ethanol (15 mL) at 78° C. for 16 hours. The reaction mixture was partitioned between ethyl acetate and water. The organic layer was separated and concentrated to give a residue. Excess sodium methoxide in ethanol was added and the mixture was heated at 75° C. for 3 hours. Water (10 mL) was added to the reaction mixture and the product was filtered and washed with water followed by ethyl acetate to give the title compound 5-amino-4-(5-methoxy(3-pyridyl))thiopheno[2,3-d]pyrimidine-6-carboxamide (0.07 g, 6.5%) as a yellow solid. ¹H NMR (400 MHz, DMSO-D6): 3.90 (s, 3H), 6.30 (s, 2H), 7.46 (s, 2H), 7.70 (s, 1H), 8.47 (m, 2H), 9.18 (s, 1H). MS m/z calculated for (M+H)⁺ 302, found 302.

### 5.2.150 5-amino-4-(5-methoxy(3-pyridyl))-2-methylthiopheno[2,3-d]pyrimidine-6-carboxamide

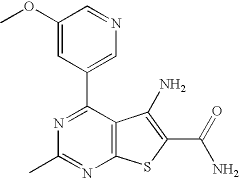

Step a: 4-hydroxy-6-(5-methoxy(3-pyridyl))-2-methylpyrimidine-5-carbonitrile. 3-Methoxy-5-formylpyridine (1.60 g, 11.7 mmol), ethylcyanoacetate (1.33 g, 11.7 mmol), acetamidine HCl (1.32 g, 14.0 mmol), and potassium carbonate (4.85 g, 35.1 mmol) were stirred in ethanol (80 mL) at 78° C. for 3 days. The solvent was removed in vacuo and 2 N HCl was added until the pH of the solution was about 4. The product was collected by filtration, washed with water and ethanol, and dried under vacuum to give 4-hydroxy-6-(5-methoxy(3-pyridyl))-2-methylpyrimidine-5-carbonitrile (1.0 g, 35%). MS m/z calculated for (M+H)⁺ 243, found 243.

Step b: 4-chloro-6-(5-methoxy(3-pyridyl))-2-methylpyrimidine-5-carbonitrile. 4-hydroxy-6-(5-methoxy(3-pyridyl))-2-methylpyrimidine-5-carbonitrile (1.0 g, 4.1 mmol) was stirred in POCl₃ (25 mL) and dioxane (25 mL) and the mixture was heated to 90° C. for 16 hours. The volatiles were removed under reduced pressure and the residue was partitioned between ethyl acetate and saturated NaHCO₃. The organic layer was separated and concentrated to give 4-chloro-6-(5-methoxy(3-pyridyl))-2-methylpyrimidine-5-carbonitrile (825 mg, 93%). MS m/z calculated for (M+H)⁺ 261, found 261.

Step c: 5-amino-4-(5-methoxy(3-pyridyl))-2-methylthiopheno[2,3-d]pyrimidine-6-carboxamide. 4-chloro-6-(5-methoxy(3-pyridyl))pyrimidine-5-carbonitrile (0.48 g, 1.83 mmol), 2-mercaptoacetamide (0.175 g, 1.92 mmol), and potassium carbonate (0.530 g, 3.8 mmol) were stirred in ethanol (15 mL) at 78° C. for 16 hours. The solvent was evaporated and water (10 mL) was added. The crude product was filtered and purified using reverse-phase preparative HPLC to give the title compound 5-amino-4-(5-methoxy(3-pyridyl))-2-methylthiopheno[2,3-d]pyrimidine-6-carboxamide (0.01 g, 1.7%) as a yellow solid. ¹H NMR (400 MHz, DMSO-D6): δ 2.75 (s, 3H), 3.89 (s, 3H), 6.25 (s, 2H), 7.38 (s, 2H), 7.65 (m, 1H), 8.40 (d, J=1.6 Hz, 1H), 8.48 (d, J=3.2, 1H). MS m/z calculated for (M+H)⁺ 316, found 316.

### 5.2.151 2,5-diamino-4-(5-methoxy(3-pyridyl))thiopheno[2,3-d]pyrimidine-6-carboxamide

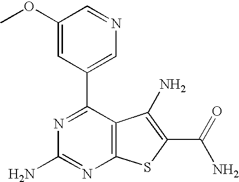

Step a: 2-amino-4-hydroxy-6-(5-methoxy(3-pyridyl))pyrimidine-5-carbonitrile. 3-Methoxy-5-formylpyridine (5.00 g, 36.4 mmol), ethylcyanoacetate (4.12 g, 36.4 mmol), guanidine hydrochloride (3.53 g, 37 mmol), and potassium carbonate (6.02 g, 43.7 mmol) were stirred in ethanol (100 mL) at 78° C. for 3 days. The solid was filtered and washed with ethanol and dried to give the title compound 2-amino-4-hydroxy-6-(5-methoxy(3-pyridyl))pyrimidine-5-carbonitrile (7.1 g, 80.2%). MS m/z calculated for (M+H)⁺ 244, found 244.

Step b: 2-amino-4-chloro-6-(5-methoxy(3-pyridyl))pyrimidine-5-carbonitrile. 2-amino-4-hydroxy-6-(5-methoxy(3-pyridyl))pyrimidine-5-carbonitrile (7.10 g, 29.2 mmol) was stirred in POCl₃ (50 mL) and the mixture was heated to 90° C. for 3 hours. The volatiles were removed under reduced pressure and the residue was partitioned between ethyl acetate and saturated NaHCO₃. The organic layer was separated, dried over Na₂SO₄, and concentrated to give 2-amino-4-chloro-6-(5-methoxy(3-pyridyl))pyrimidine-5-carbonitrile (2.1 g, 27.5%). MS m/z calculated for (M+H)⁺ 262, found 262.

Step c: 2,5-diamino-4-(5-methoxy(3-pyridyl))thiopheno[2,3-d]pyrimidine-6-carboxamide. 2-amino-4-chloro-6-(5-methoxy(3-pyridyl))pyrimidine-5-carbonitrile (1.5 g, 5.75 mmol), 2-mercaptoacetamide (0.524 g, 5.75 mmol), and diisopropylethylamine (2 mL) were stirred in ethanol (20 mL) at 70° C. for 18 hours. The reaction mixture was partitioned between ethyl acetate and water. The organic layer was separated and concentrated to give a residue. Excess sodium methoxide in ethanol was added and the mixture was heated at 78° C. for 3 hours. Water (10 mL) was added to the reaction mixture and the product was filtered and washed with water followed by ethyl acetate and ethanol to give the title compound 2,5-diamino-4-(5-methoxy(3-pyridyl))thiopheno[2,3-d]pyrimidine-6-carboxamide (0.475 g, 26.1%) as a yellow solid. ¹H NMR (300 MHz, DMSO-D6): 2.59 (s, 3H), 6.17 (s, 2H), 7.11 (s, 2H), 7.34 (s, 2H), 7.67 (m, 1H), 8.42 (d, J=1.2 Hz, 1H), 8.53 (d, J=2.7, 1H). MS m/z calculated for (M+H)⁺ 317, found 317.

### 5.2.152 5-amino-2-(cyclopropylamino)-4-(phenylamino)thiopheno[2,3-d]pyrimidine-6-carboxamide

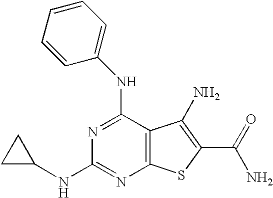

Step a: 4,6-dichloro-2-(cyclopropylamino)pyrimidine-5-carbaldehyde. Cyclopropyl amine (10.0 g, 175 mmol), 4,6-dihydroxy-2-methylmercaptopyrimidine (10.0 g, 63.2 mmol) and MeOH (60 ml) were combined and stirred at 40° C. for 4 days. The solid was filtered and washed with MeOH (15 mL) and dried under reduced pressure to give crude 2-(cyclopropylamino)pyrimidine-4,6-diol (˜7 g). In a separate flask, POCl₃ (60 mL) was cooled to 0° C. and DMF (18 mL) was added. The solution was stirred for 1 hour at 0° C. then 30 minutes at room temperature. 2-(cyclopropylamino)pyrimidine-4,6-diol (about 7 g, crude, 41.9 mmol) was added to the solution and the resulting reaction mixture stirred at room temperature for 45 minutes followed by stirring at 100° C. for 16 hours. The solution was concentrated under reduced pressure and the remaining oil was poured onto ice. The solution was neutralized with 5% NaHCO₃ solution and the crude product was extracted into ethyl acetate and dried over sodium sulfate. The solvent was remove under vacuum to give about 80% pure 4,6-dichloro-2-(cyclopropylamino)pyrimidine-5-carbaldehyde (7 g, 47.8%).

Step b: [4,6-dichloro-5-((hydroxyimino)methyl)pyrimidin-2-yl]cyclopropylamine. 4,6-dichloro-2-(cyclopropylamino)pyrimidine-5-carbaldehyde (7.00 g, 30.2 mmol) was added to glacial acetic acid (60 mL) and stirred at room temperature for 10 minutes. Hydroxylamine hydrochloride (2.10 g, 30.17 mmol) was added and the mixture was stirred at 40° C. for 3 hours and 50° C. for 1 hour. The solution was poured over ice and extracted with ethyl acetate. The organic layer was washed with 5% NaHCO₃ and dried over sodium sulfate. The solvent was removed under reduced pressure to give [4,6-dichloro-5-((hydroxyimino)methyl)pyrimidin-2-yl]cyclopropylamine (6 g, 80.5%) as a semi-pure product. MS m/z calculated for (M+H)⁺ 247, found 247.

Step c: 4,6-dichloro-2-(cyclopropylamino)pyrimidine-5-carbonitrile. [4,6-dichloro-5-((hydroxyimino)methyl)pyrimidin-2-yl]cyclopropylamine (6 g, 24.3 mmol) was added to POCl₃ (45 mL) and the mixture was heated at 105° C. for 3.5 hours. The solvent was concentrated under reduced pressure and the remaining oil was poured onto ice and neutralized with 5% NaHCO₃ solution. The product was extracted into ethyl acetate and dried over sodium sulfate. The product was purified using silica-gel chromatography (25% hexane in ethyl acetate) to give 4,6-dichloro-2-(cyclopropylamino)pyrimidine-5-carbonitrile (3.0 g, 54%) as a tan solid. ¹H NMR (400 MHz, DMSO-D6): 0.69 (m, 2H), 1.03 (m, 2H), 2.67 (m, 1H). MS m/z calculated for (M+H)⁺ 229, found 229.

Step d: 2-[6-chloro-5-cyano-2-(cyclopropylamino)pyrimidin-4-ylthio]acetamide. 4,6-dichloro-2-(cyclopropylamino)pyrimidine-5-carbonitrile (1 g, 4.39 mmol) was dissolved in THF (15 mL) followed by addition of diisopropylethylamine (764 ul, 4.40 mmol) and mercaptoacetamide (0.4 g, 4.39 mmol). The reaction mixture was stirred at room temperature for 18 hours and the solvent was removed under reduced pressure. The remaining solid was washed with water (25 mL) and dried to give 2-[6-chloro-5-cyano-2-(cyclopropylamino)pyrimidin-4-ylthio]acetamide (1.1 g, 88.4%).

Step e: 5-amino-2-(cyclopropylamino)-4-(phenylamino)thiopheno[2,3-d]pyrimidine-6-carboxamide. 2-[6-chloro-5-cyano-2-(cyclopropylamino)pyrimidin-4-ylthio]acetamide (0.3 g, 1.06 mmol) was added to a glass vial along with aniline (0.35 g, 3.7 mmol). The reaction was heated at 90° C. for 16 hours. DMF (2 mL) was added and the product was precipitated with the addition of water (20 mL). The solid was filtered, washed with water and ethanol (2 mL), and dried to give about 0.3 grams of crude uncyclized material. The residue was dissolved in ethanol (25 mL) and sodium methoxide (0.15 g, 2.78 mmol) was added. The mixture was heated at reflux for 4 hours then quenched with water (40 mL). The crude material was extracted into ethyl acetate and dried over sodium sulfate. The solvent was removed under reduced pressure and the residue was dissolved in DMSO (4 mL). The product was purified using reverse-phase preparative HPLC (20-60% ACN, 0.1% TFA) to give 5-amino-2-(cyclopropylamino)-4-(phenylamino)thiopheno[2,3-d]pyrimidine-6-carboxamide (0.03 g, 8.8%) as a light yellow solid. ¹H NMR (400 MHz, DMSO-D6): 0.50 (m, 2H), 0.67 (m, 2H), 2.70 (m, 1H), 6.94-7.08 (m, 5H), 7.34 (m, 3H), 7.83 (bm, 2H), 8.38 (bs, 1H). MS t/z calculated for (M+H)⁺ 341, found 341.

### 5.2.153 5-amino-4-[(3,4-dichlorophenyl)amino]-2-(cyclopropylamino)thiopheno[2,3-d]pyrimidine-6-carboxamide

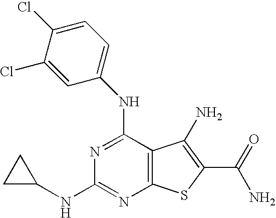

Step a: 2-{6-[(3,4-dichlorophenyl)amino]-5-cyano-2-(cyclopropylamino)pyrimidin-4-ylthio}acetamide. 4,6-dichloro-2-(cyclopropylamino)pyrimidine-5-carbonitrile (0.35 g, 1.53 mmol), 3,4-dichloroaniline (0.25 g, 1.53 mmol) and diisopropylethylamine (0.5 mL) were combined in THF (3 mL) and heated at 100° C. for 30 minutes in a microwave reactor. Without workup or purification, the mixture was transferred to a flask and THF (10 mL), DMF (2 mL), and diisopropylethylamine (0.25 mL) were added. Mercaptoacetamide (0.11 g, 1.2 mmol) was added and the mixture was stirred at 50° C. for 6 hours. The volatiles were removed in vacuo and water (20 mL) was added. The crude product was collected using filtration and washed with ethanol (10 mL). The product was dried under reduced pressure to give crude 2-{6-[(3,4-dichlorophenyl)amino]-5-cyano-2-(cyclopropylamino)pyrimidin-4-ylthio}acetamide (0.4 g, 63.8%). MS m/z calculated for (M+H)⁺ 409, found 409.

Step b: 5-amino-4-[(3,4-dichlorophenyl)amino]-2-(cyclopropylamino)thiopheno[2,3-d]pyrimidine-6-carboxamide. 2-{6-[(3,4-dichlorophenyl)amino]-5-cyano-2-(cyclopropylamino)pyrimidin-4-ylthio}acetamide (0.4 g, 0.98 mmol) was added to ethanol (20 mL) and sodium methoxide (0.2 g, 3.7 mmol) and stirred at reflux for 4 hours. The reaction was quenched with water (100 mL) and the crude product was extracted into ethyl acetate and dried over sodium sulfate. The solvent was evaporated under reduced pressure and the residue was dissolved in DMSO (10 mL). The product was purified using reverse-phase preparative HPLC (30-80% ACN, 0.1% TFA) to give 5-amino-4-[(3,4-dichlorophenyl)amino]-2-(cyclopropylamino)thiopheno[2,3-d]pyrimidine-6-carboxamide (0.055 g, 13.4%) as a light yellow solid. ¹H NMR (400 MHz, DMSO-D6): 0.52 (m, 2H), 0.74 (m, 2H), 2.68 (m, 1H), 6.95 (bs, 2H), 7.07 (bs, 2H), 7.58 (m, 2H), 7.74 (m, 1H), 8.59 (bs, 2H). MS m/z calculated for (M+H)⁺ 409, found 409.

### 5.2.154 5-amino-4-(5-chloro-3-hydroxyphenyl)thiopheno[2,3-d]pyrimidine-6-carboxamide

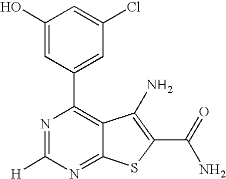

Step a: 6-(3-chloro-5-methoxyphenyl)-4-hydroxy-2-methylthiopyrimidine-5-carbonitrile. 3-chloro-5-methoxybenzaldehyde (6.52 g, 38.13 mmol), ethyl cyanoacetate (4.31 g, 38.13 mmol), methylthiocarboxamidine (10.61 g, 38.13 mmol), and potassium carbonate (5.26 g, 38.13 mmol) were refluxed in ethanol (100 mL) overnight. The stirbar was removed and water was added. The volatiles were removed and the aqueous layer was extracted with ethyl acetate, dried with sodium sulfate, and concentrated to give 12 g (100%) of the title compound as a brown oil. MS m/z calculated for (M+H)⁺ 308, found 308.

Step b: 4-chloro-6-(3-chloro-5-methoxyphenyl)-2-methylthiopyrimidine-5-carbonitrile. 6-(3-chloro-5-methoxyphenyl)-4-hydroxy-2-methylthiopyrimidine-5-carbonitrile (11.71 g, 38.14 mmol) was dissolved in dioxane (200 mL) and POCl₃ (40 mL) was added. After the addition of DMF (3 mL), the resulting reaction mixture was heated at 90° C. under a reflux condenser for 4 hours and then cooled to room temperature. The volatiles were removed and the remaining reaction mixture was added to ice and neutralized with saturated NaHCO₃. The aqueous layer was extracted with ethyl acetate, dried with sodium sulfate, and concentrated. The residue was purified using flash chromatography (1:1 EtOAc/hexane) to give 3.5 g (28%) of the title compound as an off-white solid. ¹H NMR (300 MHz, DMSO-D6) δ ppm 2.65 (s, 3H) 3.87 (s, 3H) 7.37 (t, J=2.20 Hz, 1H) 7.44-7.50 (m, 1H) 7.55 (t, J=1.65 Hz, 1H). MS m/z calculated for (M+H)⁺ 326, found 326.

Step c: 5-amino-4-(3-chloro-5-methoxyphenyl)-2-methylthiothiopheno[2,3-d]pyrimidine-6-carboxamide. 4-chloro-6-(3-chloro-5-methoxyphenyl)-2-methylthiopyrimidine-5-carbonitrile (3.5 g, 10.77 mmol), 2-mercaptoacetamide (980 mg, 10.77 mmol), Na₂CO₃ (1140 mg, 10.77 mmol), and ethanol (60 mL) were combined in a flask, stirred vigorously, and heated at 70° C. under a reflux condenser for 5 hours. The reaction mixture was cooled to room temperature and water was added. The resulting precipitate was filtered and washed with water to give a white solid that was used without purification in the next step. 2-[6-(3-chloro-5-methoxyphenyl)-5-cyano-2-methylthiopyrimidin-4-ylthio]acetamide was dissolved in ethanol (15 mL). Freshly prepared NaOEt (17.8 mmol) in ethanol (10 mL) was added and the resulting mixture stirred at 70° C. for 1 hour. The reaction mixture was cooled to room temperature and quenched with saturated NaHCO₃. The resulting yellow precipitate was collected by filtration to give 3.48 g (85%) of the title compound as a yellow solid. ¹H NMR (300 MHz, DMSO-D6) δ ppm 2.60 (s, 3H) 3.84 (s, 3H) 6.21 (s, 2H) 7.12-7.22 (m, 1H) 7.23-7.31 (m, 2H) 7.34 (s, 2H). MS m/z calculated for (M+H)⁺ 381, found 381.

Step d: 5-amino-4-(3-chloro-5-methoxyphenyl)-2-(methylsulfinyl)thiopheno[2,3-d]pyrimidine-6-carboxamide. 5-amino-4-(3-chloro-5-methoxyphenyl)-2-methylthiothiopheno[2,3-d]pyrimidine-6-carboxamide (3.48 g, 9.16 mmol) was dissolved in CHCl₃ (15 mL) and the solution cooled to 0° C. mCPBA (5.0 g, 22.89 mmol) was added and the reaction mixture stirred at 0° C. for 1 hour. NaHSO₃ (10% solution) was added and the reaction mixture stirred at room temperature for 1 hour. The layers were separated and the organics washed with saturated NaHCO₃, dried with sodium sulfate, and concentrated to give 2.46 g (68%) of the title compound as a yellow powder. MS m/z calculated for (M+H)⁺ 397, found 397.

Step e: 5-amino-4-(3-chloro-5-methoxyphenyl)thiopheno[2,3-d]pyrimidine-6-carboxamidenyl)thiopheno[2,3-d]pyrimidine-6-carboxamide. 5-amino-4-(3-chloro-5-methoxyphenyl)-2-(methylsulfinyl)thiopheno[2,3-d]pyrimidine-6-carboxamide (11.0 g, 2.53 mmol) was dissolved in EtOH/CHCl₃ (1:1, 30 mL total volume). NaBH₄ (0.191 g, 5.05 mmol) was added in small portions and the reaction was allowed to stir at room temperature for 15 minutes. Water was added to the reaction and the organics were concentrated. The resulting yellow precipitate was collected by vacuum filtration and dried to provide 310 mg (37%) of the title compound as a yellow solid. MS m/z calculated for (M+H)⁺ 335, found 335.

Step f: 5-amino-4-(5-chloro-3-hydroxyphenyl)thiopheno[2,3-d]pyrimidine-6-carboxamide. 5-amino-4-(3-chloro-5-methoxyphenyl)thiopheno[2,3-d]pyrimidine-6-carboxamidenyl)thiopheno[2,3-d]pyrimidine-6-carboxamide (310 mg, 0.925 mmol) was dissolved in CH₂Cl₂ (20 mL) and BBr₃ (4.6 mL) was added. The reaction mixture was stirred at room temperature for 18 hours when an additional 2 mL of BBr₃ was added. After an additional 5 hours, the mixture was added to ice and neutralized with saturated NaHCO₃. The aqueous layer was extracted with ethyl acetate, dried with sodium sulfate, and concentrated. The residue was purified using reverse phase preparatory HPLC (10-70% CH₃CN/H₂O) and CH₃CN was removed on a rotary evaporator. The resulting aqueous mixture was extracted with EtOAc and the organic layer was washed 5 times with saturated NaHCO₃ to remove TFA. The organic layer was dried with sodium sulfate and concentrated on a rotary evaporator. The resulting residue was triturated with EtOAc/hexane and the yellow powder filtered and dried under high vacuum to give 18 mg (6%) of the title compound as a yellow solid. ¹H NMR (400 MHz, DMSO-D6) δ ppm 6.23 (s, 2H) 6.92 (s, 1H) 6.97 (s, 1H) 7.06 (s, 1H) 7.43 (s, 2H) 9.15 (s, 1H) 10.4 (s, 1H). MS m/z calculated for (M+H)⁺ 321, found 321.

### 5.2.155 5-amino-4-(3,4-dichlorophenyl)-2-{[(hydroxymethyl)cyclobutyl]amino}thiopheno[2,3-d]pyrimidine-6-carboxamide

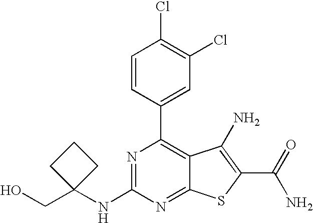

5-amino-4-(3,4-dichlorophenyl)-2-(methylsulfinyl)thiopheno[2,3-d]pyrimidine-6-carboxamide (289 mg, 0.723 mmol) and [(aminomethyl)cyclobutyl]methan-1-ol (219.5 mg, 2.17 mmol) were dissolved in DMF (5.0 mL) with stirring. The resulting mixture was heated at 90° C. under a reflux condenser for 18 hours and then cooled to room temperature. The resulting mixture was concentrated, dissolved in MeOH, and purified using reverse phase preparatory HPLC (20-70% CH₃CN/H₂O) to give 30 mg of a yellow solid. The solids were dissolved in methanol and triethylamine (800 uL) was added followed by the addition of MP carbonate resin (10 equiv). The resulting mixture was allowed to stir at 25° C. for 18 hours. The resulting mixture was filtered and the MP resin washed twice with methanol. The filtrate was concentrated on a rotary evaporator to give 19.0 mg (6.0%) of 5-amino-4-(3,4-dichlorophenyl)-2-{[(hydroxymethyl)cyclobutyl]amino}thiopheno[2,3-d]pyrimidine-6-carboxamide. ¹H NMR (400 MHz, DMSO d6) δ ppm 1.75 (m, 2H) 2.14-2.24 (m, 5H) 3.16 (d, J=5.2 Hz, 1H) 3.66-3.68 (m, 2H) 4.05 (t, J=0.8 Hz, 1H) (t, J=5.6 Hz, 1H) 7.02 (s, br, 2H) 7.57-7.59 (m, 1H) 7.75-7.81 (m, 1H) 7.85 (d, J=2.0, Hz, 1H). MS m/z calculated for (M+2, 4)⁺ 440, 442 found 440, 442.

### 5.2.156 5-amino-2-[(2-amino-tert-butyl)amino]-4-[3-({[4-(trifluoromethyl)phenyl]amino}carbonylamino)phenyl]thiopheno[2,3-d]pyrimidine-6-carboxamide

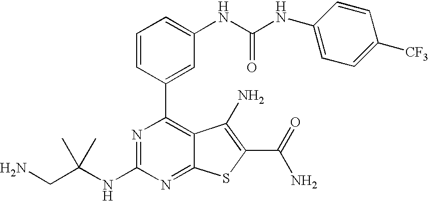

Step a: 5-amino-2-({2-[(tert-butoxy)carbonylamino]-tert-butyl}-amino)-4-(3-nitrophenyl)thiopheno[2,3-d]pyrimidine-6-carboxamide. 5-amino-2-(methylsulfinyl)-4-(3-nitrophenyl)thiopheno[2,3-d]pyrimidine-6-carboxamide (2.03 g, 5.38 mmol) and N-(2-amino-2-methylpropyl)(tert-butoxy)carboxamide (1.52 g, 8.07 mmol) were dissolved in DMF (15.0 mL) with stirring The resulting mixture was heated at 85° C. under a reflux condenser for 18 hours and then cooled to room temperature. The resulting mixture was concentrated. The resulting residue was dissolved in MeOH and purified using reverse phase preparatory HPLC (20-70% CH₃CN/H₂O) to give 230 mg (9.0%) of 5-amino-2-({2-[(tert-butoxy)carbonylamino]-tert-butyl}amino)-4-(3-nitrophenyl)thiopheno[2,3-d]pyrimidine-6-carboxamide. MS m/z calculated for (M+1)⁺ 502, found 502.

Step b: 5-amino-4-(3-aminophenyl)-2-({2-[(tert-butoxy)carbonylamino]-tert-butyl}amino)thiopheno[2,3-d]pyrimidine-6-carboxamide. 5-amino-2-({2-[(tert-butoxy)carbonylamino]-tert-butyl}amino)-4-(3-nitrophenyl)thiopheno[2,3-d]pyrimidine-6-carboxamide (184 mg, 0.367 mmol) was added to ethanol (20 mL) followed by the addition of tin chloride dihydrate (415.2 mg, 1.84 mmol). The resulting mixture was stirred at room temperature for 18 hours. The mixture was concentrated then diluted with 15 mL of water and filtered. The resulting filtrate was adjusted to pH 9 with sodium bicarbonate and extracted with ethyl acetate. The organic layers were combined, dried over MgSO₄, concentrated, and dried under high vacuum to give 127 mg (73%) of 5-amino-4-(3-aminophenyl)-2-({2-[(tert-butoxy)carbonylamino]-tert-butyl}amino)thiopheno[2,3-d]pyrimidine-6-carboxamide. MS m/z calculated for (M+1)⁺ 472, found 472.

Step c: 5-amino-2-({2-[(tert-butoxy)carbonylamino]-tert-butyl}amino)-4-[3-({[4-(trifluoromethyl)phenyl]amino}carbonylamino)phenyl]thiopheno[2,3-d]pyrimidine-6-carboxamide. 5-amino-4-(3-aminophenyl)-2-({2-[(tert-butoxy)carbonylamino]-tert-butyl}amino)thiopheno[2,3-d]pyrimidine-6-carboxamide (127 mg, 0.269 mmol) was added to dichloromethane (5 mL) followed by the addition of 4-(trifluoromethyl)benzenisocyanate (50.3 mg, 2.69 mmol). The resulting mixture was stirred at room temperature for 1.5 hours. The desired product precipitated out of solution and was collected using vacuum filtration and dried under high vacuum to give 105 mg (59%) of 5-amino-2-({2-[(tert-butoxy)carbonylamino]-tert-butyl}amino)-4-[3-({[4(trifluoromethyl)phenyl]amino}carbonylamino)phenyl]thiopheno[2,3-d]pyrimidine-6-carboxamide. MS m/z calculated for (M+1)⁺ 659, found 659.

Step d: 5-amino-2-[(2-amino-tert-butyl)amino]-4-[3-({[4-(trifluoromethyl)phenyl]amino}carbonylamino)phenyl]thiopheno[2,3-d]pyrimidine-6-carboxamide. 5-amino-2-({2-[(tert-butoxy)carbonylamino]-tert-butyl}amino)-4-[3-({[4-(trifluoromethyl)phenyl]amino}carbonylamino)phenyl]thiopheno[2,3-d]pyrimidine-6-carboxamide (105 mg, 0.159 mmol) was dissolved in CH₂Cl₂ (10 mL). Trifluoroacetic acid (1.0 mL) was added dropwise via syringe. The reaction mixture was stirred 18 hours at 25° C. The resulting crude material was dissolved in MeOH and purified using reverse phase preparatory HPLC (20-70% CH₃CN/H₂O) to give 185 mg of a yellow solid. This material was dissolved in methanol and triethylamine (800 uL) was added followed by the addition of MP carbonate resin (10 equiv). The resulting mixture was allowed to stir at 25° C. for 18 hours. The resulting mixture was filtered and the MP resin washed twice with methanol. The filtrate was concentrated on a rotary evaporator to give 36.0 mg (41%) of 5-amino-2-[(2-amino-tert-butyl)amino]-4-[3-({[4(trifluoromethyl)phenyl]amino}carbonylamino)phenyl]thiopheno[2,3-d]pyrimidine-6-carboxamide. ¹H NMR (400 MHz, CD₃OD) δ ppm 1.53 (s, 6H) 3.56 (s, 2H) 7.28 (m, 1H) 7.28-7.3 (m, 1H) 7.49-7.65 (m, 6H) 7.93 (s, 1H). MS m/z calculated for (M+1)⁺ 559, found 559.

### 5.2.157 5-amino-4-(3-{[(4-methoxyphenyl)amino]carbonylamino}phenyl)thiopheno[2,3-d]pyrimidine-6-carboxamide

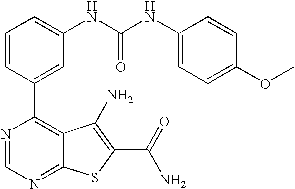

5-amino-4-(3-aminophenyl)thiopheno[2,3-d]pyrimidine-6-carboxamide (230 mg, 0.806 mmol) was dissolved in THF (10 mL) followed by the addition of 4-methoxybenzenisocyanate (120 mg, 0.806 mmol). The resulting mixture was stirred at room temperature for 2 hours. The resulting crude material was dissolved in MeOH and purified using reverse phase preparatory HPLC (20-70% CH₃CN/H₂O) to give 65 mg of a yellow solid. This material was dissolved in methanol and triethylamine (800 uL) was added followed by the addition of MP carbonate resin (10 equiv). The resulting mixture was allowed to stir at 25° C. for 18 hours. The resulting mixture was filtered and the MP resin washed twice with methanol. The filtrate was concentrated on a rotary evaporator to give 45 mg (13%) of 5-amino-4-(3-{[(4-methoxyphenyl)amino]carbonylamino}phenyl)thiopheno[2,3-d]pyrimidine-6-carboxamide. ¹H NMR (300 MHz, CD₃OD) δ ppm 3.87 (s, 3H) 6.96-6.98 (m, 2H) 7.38-7.43 (m, 3H) 7.62-7.67 (m, 1H) 7.76-7.79 (m, 1H) 7.86-7.87 (m, 1H) 9.16 (s, 1H). MS m/z calculated for (M+1)⁺ 435 found 435.

### 5.2.158 5-Amino-2-(1-hydroxy-2-methylpropan-2-ylamino)-4-(4-methoxypyridin-2-yl)thieno[2,3-d]pyrimidine-6-carboxamide

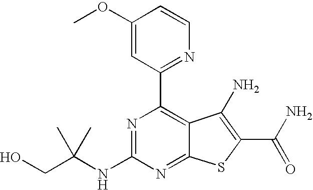

Step a: 4-Hydroxy-6-(4-methoxypyridin-2-yl)-2-(methylthio)pyrimidine-5-carbonitrile. 4-Methoxypicolinaldehyde (1.9 g, 13.85 mmol), methyl carbamimidothioate hemisulfate (1.93 g, 6.93 mmol), ethyl 2-cyanoacetate (1.57 g, 13.8 mmol) and potassium carbonate (2.30 g, 16.6 mmol) were mixed in 95 ml dry EtOH under N₂ and heated at 75° C. for two hours. The suspension was cooled to room temperature and the solids were collected by filtration. The solids were suspended in 50 ml H₂O, stirred for 30 minutes, collected by filtration, and dried overnight to give the desired product as a red solid (1.04 g, 27%). ¹H NMR (400 MHz, DMSO-D6) δ ppm 2.37 (s, 3H), 3.88 (s, 3H), 7.05 (m, 1H), 7.43 (d, J=2.54 Hz, 1H), 8.45 (d, J=5.66 Hz, 1H). MS m/z calculated for (M+H)⁺ 275.31, found 275.

Step b: 4-Chloro-6-(4-methoxypyridin-2-yl)-2-(methylthio)pyrimidine-5-carbonitrile. 4-Hydroxy-6-(4-methoxypyridin-2-yl)-2-(methylthio)pyrimidine-5-carbonitrile (0.992 g, 3.62 mmol) was dissolved in 60 ml dry dioxane under N₂ and phosphorus oxychloride (15.7 ml, 169 mmol) and a few drops of dry DMF were added. The reaction mixture was stirred overnight and the solvent evaporated. The reaction mixture was quenched by adding ice/water and the aqueous layer was extracted with EtOAc. The organic phase was dried over MgSO₄ and evaporated to give the product as a red solid (0.976 g, 92%). ¹H NMR (400 MHz, DMSO-D6) δ ppm 2.50 (s, 3H), 3.95 (s, 3H), 7.31 (d, J=2.54 Hz, 1H), 7.82 (s, 1H), 8.63 (d, J=5.66 Hz, 1H). MS m/z calculated for (M+H)⁺ 293.75, found 293.

Step c: 5-Amino-4-(4-methoxypyridin-2-yl)-2-(methylthio)thieno[2,3-d]pyrimidine-6-carboxamide. 4-Chloro-6-(4-methoxypyridin-2-yl)-2-(methylthio)pyrimidine-5-carbonitrile (0.976 g, 3.33 mmol), 2-mercaptoacetamide (0.334, 3.67 mmol) and sodium carbonate (0.389 g, 3.67 mmol) were suspended in 30 ml dry EtOH under N₂ and stirred overnight. The solids were collected using filtration and washed with a small amount of water. The solids were added to a solution of sodium (383 mg 16.65 mmol) in 10 ml dry EtOH under N₂ and heated at 70° C. for two hours. The reaction mixture was poured into water and extracted with EtOAc. The combined organic phases were dried over MgSO₄ and the solvent evaporated to give the product as a red solid (0.266 g, 23%). ¹H NMR (400 MHz, DMSO-D6) δ ppm 2.51 (s, 3H), 3.97 (s, 3H), 7.18 (m, 1H), 7.28 (m, 2H), 7.88 (m, 1H), 8.35 (m, 2H), 8.65 (m, 1H). MS m/z calculated for (M+H)⁺ 348.42, found 348.

Step d: 5-Amino-4-(4-methoxypyridin-2-yl)-2-(methylsulfinyl)thieno[2,3-d]pyrimidine-6-carboxamide. 5-Amino-4-(4-methoxypyridin-2-yl)-2-(methylthio)thieno[2,3-d]pyrimidine-6-carboxamide (236 mg, 0.679 mmol) was dissolved in 80 ml dry CHCl₃ under N₂ and cooled to 0° C. 3-Chloroperoxybenzoic acid (212 mg, 1.227 mmol) was added and the mixture was stirred at 0° C. for 75 minutes. The reaction was quenched with 10% aqueous NaHSO₃. The phases were separated and the organic phase was washed with 10% aqueous NaHCO₃. The organic phase was dried over MgSO₄ and the solvent evaporated to give the product as a red solid (0.198 g, 80%). MS m/z calculated for (M+H)⁺ 364.42, found 364. The compound was used directly for the next step.

Step e: 5-Amino-2-(1-hydroxy-2-methylpropan-2-ylamino)-4-(4-methoxypyridin-2-yl)thieno[2,3-d]pyrimidine-6-carboxamide. 5-Amino-4-(4-methoxypyridin-2-yl)-2-(methylsulfinyl)thieno[2,3-d]pyrimidine-6-carboxamide (198 mg, 0.545 mmol) and 2-amino-2-methylpropan-1-ol (486 mg, 5.45 mmol) were heated in 10 ml dry DMF under N₂ at 90° C. for 18 hours. The solvent was evaporated and the residue was suspended in DCM. The solid was collected using filtration and purified using HPLC (Method 10-100%, 30 min, 20 ml/min) to give the product as an orange solid (28 mg, 13%). ¹H NMR (400 MHz, DMSO-D6) 5 ppm 1.23 (m, 2H), 1.37 (s, 3H), 3.58 (d, J=6.05 Hz, 2H), 3.95 (s, 3H), 4.86 (m, 1H), 6.92 (s, 2H), 7.07 (s, 1H), 7.24 (m, 1H), 7.82 (s, 1H), 8.59 (d, J=5.86 Hz, 1H). MS m/z calculated for (M+H)⁺ 389.45, found 389.

### 5.2.159 5-Amino-4-[3-(difluoromethyl)phenyl]-2-ethoxythiopheno[2,3-d]pyrimidine-6-carboxamide

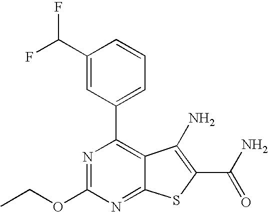

Step a: 3-(Difluoromethyl)benzaldehyde. 1-Bromo-3-(difluoromethyl)benzene (3.8 g, 18.36 mmol) was dissolved in dry THF under N₂ and cooled to −78° C. Butyllithium (11.93 ml, 19.09 mmol) was added slowly and the reaction mixture was stirred at −78° C. for one hour. N,N-dimethylformamide (1.570 ml, 20.19 mmol) was added dropwise and the reaction mixture was stirred for 30 minutes. The cold solution was poured into 320 ml of 5% aqueous NaHCO₃ and extracted with 2×160 mL EtOAc. The organic phases were dried over MgSO₄ and the solvent evaporated. The residue was purified using column chromatography (SiO₂, hexanes/EtOAc 9/1, R_{f}=0.18) to give the product as a yellow oil (2.37 g, 83%). ¹H NMR (400 MHz, DMSO-D6) δ ppm 7.18 (t, J=5.44, 1H), 7.77 (m, 1H), 7.92 (d, J=7.61, 1H), 8.09 (m, 2H), 10.09 (s, 1H). MS m/z calculated for (M+H)⁺ 156.13, found 156.

Step b: 6-[3-(Difluoromethyl)phenyl]-4-hydroxy-2-methylthiopyrimidine-5-carbonitrile. 3-(Difluoromethyl)benzaldehyde (2.57 g, 16.46 mmol), methyl carbamimidothioate hemisulfate (2.30 g, 8.23 mmol), ethyl 2-cyanoacetate (1.86 g, 16.46 mmol) and potassium carbonate (2.73 g, 19.75 mmol) were mixed in 105 ml dry EtOH under N₂ and heated at 75° C. for 16 hours. The suspension was cooled to room temperature and solids were collected by filtration. The solids were suspended in 50 ml H₂O and stirred for 30 minutes, collected by filtration. and dried overnight to give the desired product as a white solid (1.21 g, 25%). ¹H NMR (400 MHz, DMSO-D6) δ ppm 2.56 (s, 3H), 7.16 (t, J=55.6, 1H), 7.73 (m, 2H), 8.08 (br s, 2H). MS m/z calculated for (M+H)⁺ 293.29, found 293.

Step c: 6-[3-(Difluoromethyl)phenyl]-4-chloro-2-methylthiopyrimidine-5-carbonitrile. 6-[3-(Difluoromethyl)phenyl]-4-hydroxy-2-methylthiopyrimidine-5-carbonitrile (1.11 g, 3.78 mmol) was dissolved in 35 ml dry dioxane under N₂ and POCl₃ (3.52 ml, 37.8 mmol) and a few drops of dry DMF were added. The reaction mixture was heated at 90° C. for three hours. The solvent was removed on a rotary evaporator and the residue was quenched with ice and water and extracted with EtOAc. The combined organic phases were dried over MgSO₄ and the solvent was evaporated to give the product as a white solid (1.12 g, 95%). ¹H NMR (400 MHz, DMSO-D6) δ ppm 2.66 (s, 3H), 7.20 (t, J=55.44, 1H), 7.80 (m, 1H), 7.88 (m, 1H), 8.15 (br s, 2H). MS m/z calculated for (M+H)⁺ 311.74, found 311.

Step d: 5-Amino-4-[3-(difluoromethyl)phenyl]-2-ethoxythiopheno[2,3-d]pyrimidine-6-carboxamide. 6-[3-(Difluoromethyl)phenyl]-4-chloro-2-methylthiopyrimidine-5-carbonitrile (1.12 g, 3.59 mmol), 2-mercaptoacetamide (0.360, 3.95 mmol) and sodium carbonate (0.419 g, 3.95 mmol) were suspended in 35 ml dry EtOH under N₂ and the reaction mixture was heated at 70° C. for two hours. The solids were collected by filtration and washed with a small amount of water. The solids were purified using HPLC (Method 10-100%, 30 min, 20 ml/min) to give the product as a yellow solid (37 mg, 3%). ¹H NMR (400 MHz, DMSO-D6) δ ppm 1.37 (t, J=7.03, 3H), 4.45 (q, J=7.03, 2H), 6.14 (s, 2H), 7.16 (m, 3H), 7.73 (m, 1H), 7.84 (m, 3H). MS m/z calculated for (M+H)⁺ 364.37, found 364.

### 5.2.160 5-amino-4-[3-(difluoromethyl)phenyl]-2-[(2-hydroxy-tert-butyl)amino]thiopheno-[2,3-d]pyrimidine-6-carboxamide

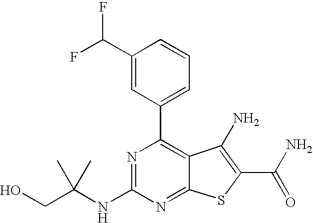

Step a: 5-Amino-4-[3-(difluoromethyl)phenyl]-2-methylthiothiopheno[2,3-d]pyrimidine-6-carboxamide. 6-[3-(Difluoromethyl)phenyl]-4-chloro-2-methylthiopyrimidine-5-carbonitrile (1.33 g, 4.27 mmol), 2-mercaptoacetamide (0.428, 4.69 mmol) and sodium carbonate (0.497 g, 4.69 mmol) were suspended in 40 ml dry EtOH under N₂ and stirred overnight. The solids were collected by filtration and washed with a small amount of water. The solids were added to a solution of sodium (900 mg 39.13 mmol) in 10 ml dry EtOH under N₂ and stirred for one hour. The reaction mixture was poured into water and extracted with EtOAc. The combined organic phases were dried over MgSO₄ and the solvent evaporated to give the product as a yellow solid (0.830 g, 53%). ¹H NMR (400 MHz, DMSO-D6) δ ppm 2.64 (s, 3H), 4.09 (s, 3H), 7.21 (m, 2H), 7.84 (m, 2H), 8.12 (m, 2H). MS m/z calculated for (M+H)⁺ 366.41, found 366.

Step b: 5-Amino-4-[3-(difluoromethyl)phenyl]-2-(methylsulfinyl)thiopheno[2,3-d]pyrimidine-6-carboxamide. 5-Amino-4-[3-(difluoromethyl)phenyl]-2-methylthiothiopheno[2,3-d]pyrimidine-6-carboxamide (780 mg, 2.129 mmol) was dissolved in 125 ml CHCl₃ under N₂ and cooled to 0° C. 3-Chloroperoxybenzoic acid (664 mg, 3.85 mmol) was added and the mixture was stirred at 0° C. for 75 minutes. The reaction was quenched with 10% aqueous NaHSO₃. The phases were separated and the organic phase was washed with 10% aqueous NaHCO₃. The organic phase was dried over MgSO₄ and the solvent evaporated to give the product as a yellow solid (0.720 g, 88%). MS m/z calculated for (M+H)⁺ 382.41, found 382. The compound was used directly for the next step.

Step c: 5-Amino-4-[3-(difluoromethyl)phenyl]-2-[(2-hydroxy-tert-butyl)amino]-thiopheno-[2,3-d]pyrimidine-6-carboxamide. 5-Amino-4-[3-(difluoromethyl)phenyl]-2-(methylsulfinyl)thiopheno[2,3-d]pyrimidine-6-carboxamide (720 mg, 1.883 mmol) and 2-amino-2-methylpropan-1-ol (1.678 g, 18.83 mmol) were heated in 30 ml dry DMF under N₂ at 90° C. for 18 hours. The solvent was evaporated and the residue was purified using HPLC (Method 10-100%, 30 min, 20 ml/min) to give the product as a yellow solid (81 mg, 11%). ¹H NMR (400 MHz, DMSO-D6) δ ppm 1.34 (s, 6H), 3.54 (m, 2H), 4.84 (m, 1H), 6.00 (br s, 2H), 7.05 (m, 4H), 7.73 (m, 4H). MS m/z calculated for (M+H)⁺ 407.44, found 407.

### 5.2.161 5-amino-4-(3-(5-(trifluoromethyl)-1H-benzo[d]imidazol-2-ylamino)phenyl)thieno[2,3-d]pyrimidine-6-carboxamide

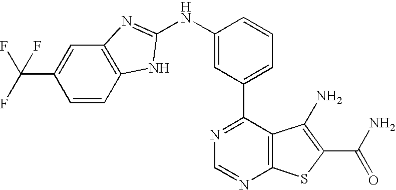

To a solution of di(1H-imidazol-1-yl)methanethione (0.150 g, 0.841 mmol) in DMF (5 mL) at 0° C., was added 5-amino-4-(3-aminophenyl)thieno[2,3-d]pyrimidine-6-carboxamide (0.240 g, 0.841 mmol). The reaction progression was followed by LC-MS. Conversion was complete after 3 hours. The reaction mixture was then warmed to room temperature and 4-(trifluoromethyl)benzene-1,2-diamine (0.148 g, 0.841 mmol) was added as a solution in DMF (2 mL). The reaction mixture was stirred at room temperature overnight. The desired product was the major product but was contaminated based on LC-MS analysis. This solution was used in the subsequent cyclization step without purification. Dicyclohexylcarbodiimide (DCC, 0.521 g, 2.52 mmol) was added as a solid and the reaction mixture was heated to 65° C. After 2 hours, all starting material was consumed. Heating was maintained for 2 additional hours, and stopped. The solvent was removed under reduced pressure. The crude residue was partitioned between water and ethyl acetate. The desired product was obtained pure after 2 consecutive semi-prep HPLC purifications using a gradient of 20-80% acetonitrile-H₂O with 0.1% TFA over 39 min. The desired fractions were combined and neutralized with 1.75 M K₂CO₃. Evaporation of the acetonitrile resulted in the formation of a bright yellow precipitate that was washed with water until the filtrate was neutral pH. The solid was dried in a vacuum oven overnight with heating to give 33 mg (6.5%) of 5-amino-4-(3-(5-(trifluoromethyl)-1H-benzo[d]imidazol-2-ylamino)phenyl)thieno[2,3-d]pyrimidine-6-carboxamide as a yellow solid. ¹H NMR (300 MHz, DMSO-d₆) δ ppm 6.37 (s, 2H) 7.2-7.65 (m, 7H) 8.05 (s, 2H) 9.17 (s, 1H) 10.01, 10.05 (s, 1H) 11.33, 11.47 (d, J=42 Hz, 1H). ¹⁹F NMR (300 MHz, DMSO-d₆) δ ppm 58.9. MS m/z calculated for (M+H)⁺ 470, found 470.

### 5.2.162 5-amino-4-(3-(5-(trifluoromethyl)benzo[d]oxazol-2-ylamino)phenyl)thieno[2,3-d]pyrimidine-6-carboxamide

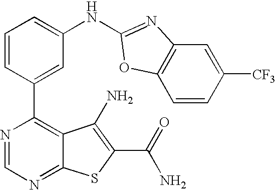

1,1′-thiocarbonyldiimidazole (0.15 g, 0.84 mmol) was dissolved in DMF (3 mL) and stirred at 0° C. under N₂ before 5-amino-4-(3-aminophenyl)thieno[2,3-d]pyrimidine-6-carboxamide (0.240 g, 0.84 mmol) was added in 5 mL of DMF and warmed to room temperature overnight. 2-amino-4-(trifluoromethyl)phenol (0.149 g, 0.84 mmol) was added in 2 mL DMF and the resulting reaction mixture stirred overnight at room temperature. 1,3-Dicyclohexylcarbodiimide (0.42 mL, 2.5 mmol) was added neat at room temperature and the reaction mixture heated at 60° C. for 3 hours. The stir bar was removed and the volatiles removed on a rotary evaporator. H₂O (10 mL) was added and the resulting precipitate was collected using vacuum filtration, washed with H₂O and dried overnight. The solid was purified using preparatory HPLC to give the title compound 5-amino-4-(3-(5-(trifluoromethyl)benzo[d]oxazol-2-ylamino)phenyl)thieno[2,3-d]pyrimidine-6-carboxamide (0.019 g, 48%) as a yellow solid. ¹H NMR (DMSO-d₆): δ 11.20 (s, 1H) 9.18 (s, 1H) 8.05 (s, 1H) 7.97 (d, J=8.79 Hz, 1H) 7.79 (s, 1H) 7.71 (d, J=8.52 Hz, 1H) 7.62 (t, J=7.96 Hz, 1H) 7.60-7.43 (m, 3H) 7.32 (d, J=7.42 Hz, 1H) 6.23 (s, 1H). MS m/z calculated for (M+H)⁺ 470, found 470.

### 5.2.163 5-amino-4-(3-nitrophenyl)-7-hydrocyclopenta[1,2-d]pyrimidine-6-carboxamide

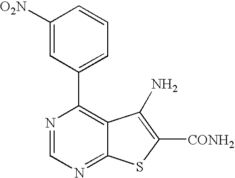

Step a: 4-hydroxy-6-(3-nitrophenyl)pyrimidine-5-carbonitrile. A suspension of m-anisaldehyde (8.0 g, 58.8 mmole), ethyl cyanoacetate (7.31 g, 64.7 mmole), methylisothiourea sulfate (8.2 g, 29.4 mmole) and potassium carbonate (8.95 g, 64.7 mmole) in ethanol (200 mL) was stirred at 80° C. for 15 hours. The resulting white slurry was diluted with ethanol (100 mL), and the precipitate was collected using vacuum filtration, rinsed with ethanol (80 mL), triturated with H₂O, and collected using vacuum filtration again. The solid was dried under high vacuum to give 8.7 g of 4-hydroxy-6-(3-nitrophenyl)pyrimidine-5-carbonitrile. MS m/z calculated for (M+H)⁺ 243, found 243.

Step b: 4-chloro-6-(3-nitrophenyl)pyrimidine-5-carbonitrile. To a stirred solution of 4-hydroxy-6-(3-nitrophenyl)pyrimidine-5-carbonitrile (3.17 g, 13.9 mmol) in 1,4-dioxane (25 mL) was added excess POCl₃ (15 mL). The resulting reaction mixture was heated at 90° C. with stirring under a reflux condenser under N₂ for 17 hours. The resulting reaction mixture was cooled to room temperature and ice was added to quench. The precipitate was collected using vacuum filtration, rinsed with a mixture DCM/Et₂O (1:1, 100 mL), and dried under high vacuum to give 3.0 g of 4-chloro-6-(3-nitrophenyl)pyrimidine-5-carbonitrile as a brown powder. MS m/z calculated for (M+H)⁺ 261, found 261.

Step c: 5-amino-4-(3-nitrophenyl)thiopheno[2,3-d]pyrimidine-6-carboxamide. 4-Chloro-6-(3-nitrophenyl)pyrimidine-5-carbonitrile (3.0 g, 11.5 mmol), 2-mercaptoacetamide (1.05 g, 11.5 mmol), diisopropylethylamine (3.01 mL, 17.3 mmol), EtOH (30 mL) and DCM (30 mL) were combined in a 200 mL round-bottom flask, stirred vigorously, and heated at room temperature under N₂ for 2.5 hours. The volatiles were removed on a rotary evaporator. The residue was triturated with 20% MeOH/CH₂Cl₂, solids were collected using vacuum filtration, washed with MeOH until colorless, and dried under high vacuum to give impure 2-[5-cyano-6-(3-nitrophenyl)pyrimidin-4-ylthio]acetamide as a white solid. MS m/z calculated for (M+H)⁺ 316, found 316. Sodium (460 mg, 19.0 mmol) was added to dry EtOH (20 mL) and the resulting mixture stirred until all of the solid had dissolved. The resulting NaOEt solution was added to a stirred mixture of the crude 2-[5-cyano-6-(3-nitrophenyl)pyrimidin-4-ylthio]acetamide in EtOH (20 mL) at room temperature. The resulting mixture was allowed to stir for 1.5 hours. The stirbar was removed and most of the solvent removed on a rotary evaporator. The residue was taken up in Et₂O and sonicated. The solids were collected using filtration and washed with more Et₂O to provide 1.5 g of the desired 5-amino-4-(3-nitrophenyl)thiopheno[2,3-d]pyrimidine-6-carboxamide. ¹H NMR (300 MHz, DMSO-D6) δ ppm 6.39 (s, 2H) 7.56 (s, 2H) 7.95 (t, J=7.8 Hz, 1H) 8.24 (d, J=7.8 Hz, 1H) 8.54 (d, J=8.1 Hz, 1H) 8.16 (s, 1H) 9.28 (s, 1H). MS m/z calculated for (M+H)⁺ 316, found 316.

### 5.2.164 5-amino-4-(3-aminophenyl)thiopheno[2,3-d]pyrimidine-6-carboxamide

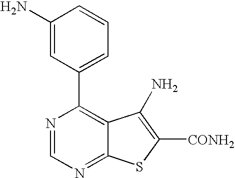

Tin chloride (3.6 g, 15.8 mmol) was added to a solution of 5-amino-4-(3-nitrophenyl)thiopheno[2,3-d]pyrimidine-6-carboxamide (1.0 g, 3.17 mmol) in EtOH/DMF (3:1, 20 mL). The resulting brown suspension was heated at 40° C. for 3 hours. The solvents were removed under reduced pressure. H₂O (15 mL) was added and the mixture filtered through Celite. The filtrate was neutralized with saturated NaHCO₃, extracted 3 times with EtOAc, dried over MgSO₄, filtered, and concentrated under reduced pressure to provide 0.7 g of the desired 5-amino-4-(3-aminophenyl)thiopheno[2,3-d]pyrimidine-6-carboxamide as a yellow powder. MS m/z calculated for (M+H)⁺ 286, found 286.

### 5.2.165 5-amino-4-[3-(2-phenylacetylamino)phenyl]thiopheno[2,3-d]pyrimidine-6-carboxamide

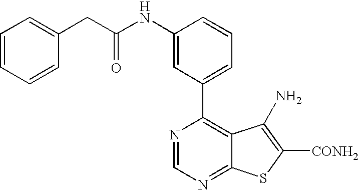

A mixture of 5-amino-4-(3-aminophenyl)thiopheno[2,3-d]pyrimidine-6-carboxamide (250 mg, 0.87 mmol), phenylacetyl chloride (122 μL, 0.92 mmol), diisopropylethylamine (160 μL, 0.92 mmol) in anhydrous THF (5 mL) was allowed to stir at room temperature for 22 hours. The crude solution was diluted with DMSO (5 mL) and directly purified using reverse phase preparatory HPLC (10-100% CH₃CN/H₂O, 0.1% TFA). CH₃CN was removed on a rotary evaporator, and the resulting TFA solution was neutralized with 1.75 M K₂CO₃. The desired product crashed out of solution and was collected using vacuum filtration, washed with H₂O, and dried under high vacuum to provide 120 mg of 5-amino-4-[3-(2-phenyl-acetylamino)phenyl]thiopheno[2,3-d]pyrimidine-6-carboxamide as a yellow powder. MP=255-258° C. ¹H NMR (300 MHz, DMSO-D6) δ ppm 3.67 (m, 2H) 6.24 (s, 2H) 7.34 (m, 9H), 7.81 (s, 1H) 7.93 (s, 1H) 9.13 (s, 1H). MS m/z calculated for (M+H)⁺ 404, found 404.

### 5.2.166 5-amino-4-{3-[(4-pyridylamino)carbonylamino]phenyl}-thiopheno[2,3-d]pyrimidine-6-carboxamide

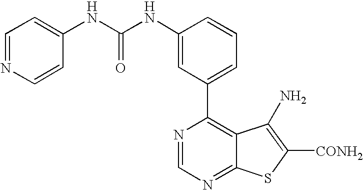

A solution of 5-amino-4-(3-aminophenyl)thiopheno[2,3-d]pyrimidine-6-carboxamide (200 mg, 0.70 mmol), 4-nitrophenyl chloroformate (142 mg, 0.70 mmol) and triethylamine (98 μL, 0.70 mmol) in anhydrous THF (5 mL) was allowed to stir at room temperature for 1 hour before a solution of 4-aminopyridine (66 mg, 0.70 mmol) and triethylamine (98 μL, 0.70 mmol) was added to it. The resulting reaction mixture was allowed to stir at room temperature for 16 hours. The volatiles were removed under reduced pressure, and the crude product was dissolved in DMSO and purified using reverse phase preparatory HPLC (10-100% CH₃CN/H₂O, 0.1% TFA). CH₃CN was removed on a rotary evaporator, and the resulting TFA solution was neutralized with 1.75 M K₂CO₃. The desired product crashed out of solution and was collected using vacuum filtration, washed with H₂O, and dried under high vacuum to provide 43 mg of 5-amino-4-{3-[(4-pyridylamino)carbonylamino]phenyl}-thiopheno[2,3-d]pyrimidine-6-carboxamide as a yellow powder. MP=177-179° C. ¹H NMR (300 MHz, DMSO-D6) δ ppm 6.27 (s, 2H) 7.31 (d, J=6.8 Hz, 1H) 7.43-7.45 (m, 2H) 7.53 (t, J=7.6 Hz, 1H) 7.66 (d, J=8.0 Hz, 1H) 7.81 (s, 1H) 8.36 (d, J=4.8 Hz, 2H) 9.16 (d, J=6.0 Hz, 2H) 9.22 (s, 1H). MS m/z calculated for (M+H)⁺ 406, found 406.

### 5.2.167 5-amino-4-{3-[(3-pyridylamino)carbonylamino]phenyl}-thiopheno[2,3-d]pyrimidine-6-carboxamide

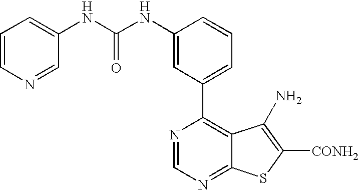

A solution of 5-amino-4-(3-aminophenyl)thiopheno[2,3-d]pyrimidine-6-carboxamide (200 mg, 0.70 mmol), 4-nitrophenyl chloroformate (142 mg, 0.70 mmol) and triethylamine (98 μL, 0.70 mmol) in anhydrous THF (5 mL) was allowed to stir at room temperature for 1 hour before a solution of 3-aminopyridine (66 mg, 0.70 mmol) and triethylamine (98 μL, 0.70 mmol) was added to it. The resulting reaction mixture was allowed to stir at room temperature for 16 hours. The volatiles were removed under reduced pressure, and the crude product was dissolved in DMSO and purified using reverse phase preparatory HPLC (10-100% CH₃CN/H₂O, 0.1% TFA). CH₃CN was removed on a rotary evaporator, and the resulting TFA solution was neutralized with 1.75 M K₂CO₃. The desired product crashed out of solution and was collected using vacuum filtration, washed with H₂O, and dried under high vacuum to provide 25 mg of 5-amino-4-{3-[(3-pyridylamino)carbonylamino]phenyl}-thiopheno[2,3-d]pyrimidine-6-carboxamide as a yellow powder. MP=199-200° C. ¹H NMR (300 MHz, DMSO-D6) δ ppm 6.27 (s, 2H) 7.28 (m, 1H) 7.42 (m, 1H) 7.52 (s, 1H) 7.64 (m, 1H) 7.81 (s, 1H) 7.95 (m, 1H) 8.20 (s, 1H) 8.60 (s, 1H) 8.95 (s, 1H) 9.11 (m, 2H). MS m/z calculated for (M+H)⁺ 406, found 406.

### 5.2.168 5-amino-4-{3-[(2-pyridylamino)carbonylamino]phenyl}-thiopheno[2,3-d]pyrimidine-6-carboxamide

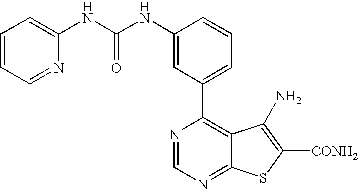

A solution of 5-amino-4-(3-aminophenyl)thiopheno[2,3-d]pyrimidine-6-carboxamide (200 mg, 0.70 mmol), 4-nitrophenyl chloroformate (142 mg, 0.70 mmol) and triethylamine (98 μL, 0.70 mmol) in anhydrous THF (5 mL) was allowed to stir at room temperature for 1 hour before a solution of 2-aminopyridine (66 mg, 0.70 mmol) and triethylamine (98 L, 0.70 mmol) was added to it. The resulting reaction mixture was allowed to stir at room temperature for 16 hours. The volatiles were removed under reduced pressure, and the crude product was dissolved in DMSO and purified using reverse phase preparatory HPLC (110-100% CH₃CN/H₂O, 0.1% TFA). CH₃CN was removed on a rotary evaporator, and the resulting TFA solution was neutralized with 1.75 M K₂CO₃. The desired product crashed out of solution and was collected using vacuum filtration, washed with H₂O, and dried under high vacuum to provide 21 mg of 5-amino-4-{3-[(2-pyridylamino)carbonylamino]phenyl}-thiopheno[2,3-d]pyrimidine-6-carboxamide as a yellow powder. MP=° C. ¹H NMR (300 MHz, DMSO-D6) δ ppm 7.15 (t, J=7.6 Hz, 1H) 7.28-7.36 (m, 3H) 7.48-7.57 (m, 4H) 8.03 (s, 1H) 8.17 (d, J=8.0 Hz, 1H) 8.41 (s, 2H). MS m/z calculated for (M+H)⁺ 406, found 406.

### 5.2.169 5-amino-4-[3-({[3-(trifluoromethyl)phenyl]amino}-carbonylamino)phenyl]thiopheno[2,3-d]pyrimidine-6-carboxamide

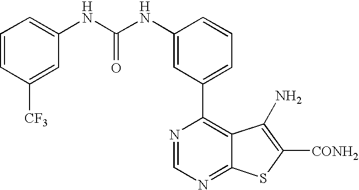

A mixture of 5-amino-4-(3-aminophenyl)thiopheno[2,3-d]pyrimidine-6-carboxamide (225 mg, 0.79 mmol), α,α,α,-trifluoro-m-tolylisocyanate (110 μL, 0.79 mmol) in anhydrous THF (5 mL) was allowed to stir at room temperature for 22 hours. The crude solution was diluted with DMSO (5 mL) and directly purified using reverse phase preparatory HPLC (10-100% CH₃CN/H₂O, 0.1% TFA). CH₃CN was removed on a rotary evaporator, and the resulting TFA solution was neutralized with 1.75 M K₂CO₃. The desired product crashed out of solution and was collected using vacuum filtration, washed with H₂O, and dried under high vacuum to provide 37 mg of 5-amino-4-[3-({[3-(trifluoromethyl)phenyl]amino}-carbonylamino)phenyl]thiopheno[2,3-d]pyrimidine-6-carboxamide as a yellow powder. MP=201-203° C. ¹H NMR (400 MHz, DMSO-D6) δ ppm 6.27 (s, 2H) 7.27-7.32 (m, 2H) 7.42 (bs, 2H) 7.52-7.54 (m, 3H) 7.64-7.66 (m, 1H) 7.83 (d, J=4.0 Hz, 1H) 8.01 (s, 1H) 9.15 (s, 1H). MS m/z calculated for (M+H)⁺ 473, found 473.

### 5.2.170 5-amino-4-[3-({[2-(trifluoromethyl)phenyl]amino}-carbonylamino)phenyl]thiopheno[2,3-d]pyrimidine-6-carboxamide

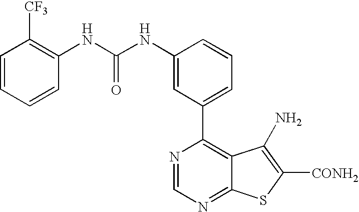

A mixture of 5-amino-4-(3-aminophenyl)thiopheno[2,3-d]pyrimidine-6-carboxamide (210 mg, 0.73 mmol), α,α,α,-trifluoro-o-tolylisocyanate (104 μL, 0.73 mmol) in anhydrous THF (5 mL) was allowed to stir at room temperature for 22 hours. The crude solution was diluted with DMSO (5 mL) and directly purified using reverse phase preparatory HPLC (10-100% CH₃CN/H₂O, 0.1% TFA). CH₃CN was removed on a rotary evaporator, and the resulting TFA solution was neutralized with 1.75 M K₂CO₃. The desired product crashed out of solution and was collected using vacuum filtration, washed with H₂O, and dried under high vacuum to provide 120 mg of 5-amino-4-[3-({[2-(trifluoromethyl)phenyl]amino}-carbonylamino)phenyl]thiopheno[2,3-d]pyrimidine-6-carboxamide as a yellow powder. MP=172-175° C. ¹H NMR (400 MHz, DMSO-D6) δ ppm 6.27 (s, 2H) 7.29 (t, J=6.8 Hz, 2H) 7.41 (s, 1H) 7.53 (t, J=8.0 Hz, 1H) 7.62-7.70 (m, 3H) 7.81 (s, 1H) 7.93 (d, J=8.4 Hz, 1H) 8.16 (s, 1H) 9.14 (s, 1H). MS m/z calculated for (M+H)⁺ 473, found 473.

### 5.2.171 Additional Representative Compounds

The following additional representative Compounds were prepared using methods described herein.

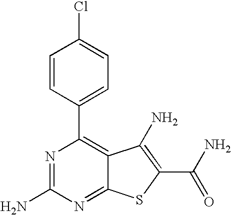

2,5-Diamino-4-(4-chloro-phenyl)-thieno[2,3-d]pyrimidine-6-carboxylic acid amide (DMSO-d6) δ 7.60 (s, 4H), 7.19 (br s, 2H), 6.98 (br s, 2H), 6.03 (br s, 2H); MH+ 320; m.p. 270; Calcd for C₁₃H₁₀N₅OSCl.0.5H₂O: C, 47.49; H, 3.37; N, 21.30 Found C, 47.30; H, 3.02; N, 21.09.

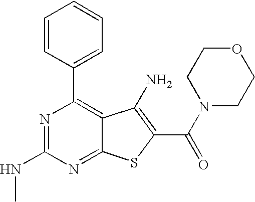

(5-Amino-2-methylamino-4-phenyl-thieno[2,3d]pyrimidin-6-yl)-morpholin-4-yl-methanone (CD₃CN) δ 7.56 (br. s, 4H), 6.04 (br. s, 1H), 5.35 (br. s, 2H), 6.03 (br. s, 2H,) 3.63 (m, 8H), 2.97 (d, 3H, J=4.8 Hz); MH+ 370.

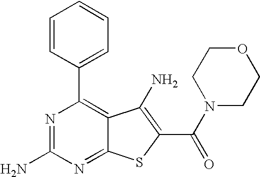

(2,5-Diamino-4-phenyl-thieno[2,3d]pyrimidin-6-yl)-morpholin-4-yl-methanone (CD3CN) δ 7.57 (m, 5H), 5.60 (br. s, 2H), 5.25 (br. s, 2H), 3.74 (m, 8H); MH+ 356.

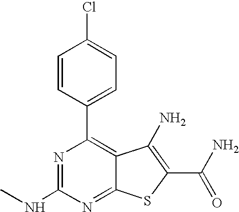

2-Aminomethyl-5-amino-4-(4-chloro-phenyl)-thieno[2,3-d]pyrimidine-6-carboxylic acid amide (DMSO) δ 7.72 (br s, 1H), 7.60 (m, 4H), 6.98 (s, 2H), 6.02 (br s, 2H), 2.87 (d, 3H, J=4.8 Hz); MH+ 334; m.p>200.

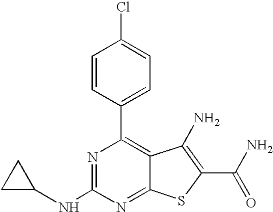

5-amino-2-cyclopropylamino-4-(4-chloro-phenyl)-thieno[2,3-d]pyrimidine-6-carboxylic acid amide (DMSO) δ 7.96 (br s, 1H), 7.61 (s, 4H), 7.00 (s, 2H), 6.03 (br s, 2H), 2.81 (sextet, 1H, J=3.6 Hz), 0.70 (q, 2H, J=5.2 Hz), 0.52 (m, 2H); MH+ 360; m.p.>200.

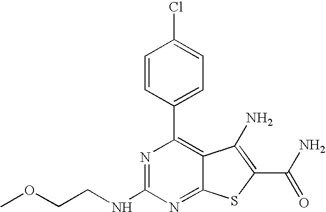

5-Amino-4-(4-chlorophenyl)-2-(2-methoxyethylamino)-thieno[2,3-d]pyrimidine-6-carboxylic acid amide (DMSO) δ 7.77 (br s, 1H), 7.61 (s, 4H), 6.99 (s, 2H), 6.02 (br s, 2H), 3.50 (m, 4H), 3.26 (s, 3H); MH+ 378; m.p.>200.

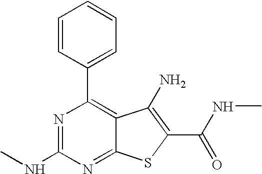

5-Amino-2-methylamino-4-phenyl-thieno[2,3d]pyrimidine-6-carboxylic acid methylamide (DMSO) δ 7.69 (m, 1H), 7.56 (br. s, 4H), 7.45 (d, 1H, J=4.8 Hz), 5.88 (br.s, 2H), 2.87 (d, 3H, J=4.8 Hz), 2.68 (d, 3H, J=4.5 Hz); MH+ 314; m.p. 266-267.

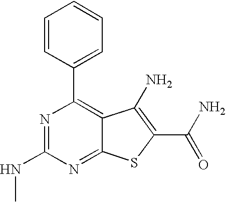

5-Amino-2-methylamino-4-phenyl-thieno[2,3d]pyrimidine-6-carboxylic acid amide (CDCl3) δ 7.65 (s, 5H), 6.10 (br. s, 2H); 5.56 (br. s, 1H); 5.12 (br. s, 2H), 3.19 (d, 3H, J=5.1 Hz); MH+ 300.

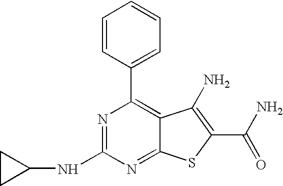

5-Amino-2-cyclopropylamino-4-phenyl-thieno[2,3d]pyrimidine-6-carboxylic acid amide (CDCl3) δ 7.65 (m, 5H), 6.05 (br. s, 2H), 5.51 (br. s, 1H), 5.15 (br. s, 2H), 2.91 (m, 1H), 0.87 (m, 2H), 0.60 (m, 2H); MH+ 326.

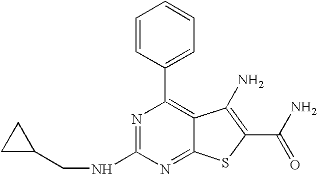

5-Amino-2-(cyclopropylmethyl-amino)-4-phenyl-thieno[2,3d]pyrimidine-6-carboxylic acid amide (DMSO) δ 7.87 (br. s, 1H), 7.57 (br. s, 5H), 6.96 (br. s, 2H), 5.97 (br. s, 2H), 3.20 (m, 2H), 1.05 (m, 1H), 0.42 (m, 2H), 0.22 (m, 2H); MH+ 340.

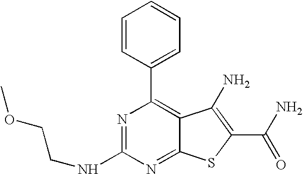

5-Amino-2-(2-methoxy-ethylamino)-4-phenyl-thieno[2,3d]pyrimidine-6-carboxylic acid amide (CDCl3) δ 7.66 (m, 5H); 6.13 (br. s, 2H); 5.90 (br. s, 1H), 5.27 (br. s, 2H), 3.82 (m, 2H), 3.70 (m, 2H), 3.49 (s, 3H); MH+ 344.

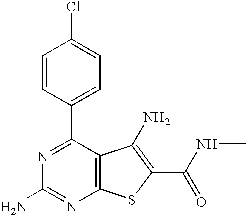

2,5-diamino-4-(4-chloro-phenyl)-thieno[2,3d]pyrimidine-6-carboxylic acid methylamide (CDCl3) δ 7.53 (m, 4H), 5.91 (br s, 2H), 5.30 (br s, 1H), 5.29 (br s, 2H), 2.94 (d, 3H, 4.8 Hz); MH+ 334; m.p.>200.

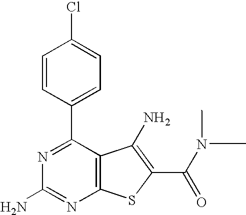

2,5-Diamino-4-(4-chlorophenyl)-thieno[2,3-d]pyrimidine-6-carboxylic acid dimethylamide (CDCl3) δ 7.53 (dd, 4H, J1=8.8 Hz, J2=2.4 Hz), 5.65 (br s, 2H), 5.29 (br s, 2H), 5.29 (br s, 2H), 3.17 (s, 3H); MH+ 348; m.p.>200.

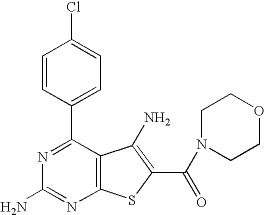

[2,5-Diamino-4-(4-chlorophenyl)-thieno[2,3-d]pyrimidin-6-yl]-morpholin-4-yl-methanone (CDCl3) δ 7.53 (dd, 4H, J1=9.2 Hz, J2=1.2 Hz), 5.56 (br s, 2H), 5.26 (br s, 2H), 3.74 (m, 8H); MH+ 390; m.p.>200.

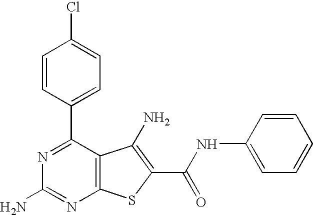

2,5-Diamino-4-(4-chlorophenyl)-thieno[2,3-d]pyrimidine-6-carboxylic acid phenyl amide (CDCl3) δ 7.55 (m, 6H), 7.35 (t, 2H, J=7.6 Hz), 7.13 (t, 1H, J=7.6 Hz), 6.98 (br s, 1H), 6.06 (br s, 2H), 5.29 (br s, 2H), 5.33 (br s, 2H); MH+ 396; m.p.>200.

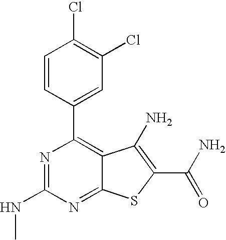

5-Amino-4-(3,4-dichloro-phenyl)-2-methylamino-thieno[2,3d]pyrimidine-6-carboxylic acid amide (CDCl3) δ 7.83 (m, 1H), 7.72 (m, 1H), 7.57 (m, 1H), 6.08 (br. s, 2H), 5.50 (br. s, 1H), 5.30 (br. s, 2H), 3.20 (d, 3H, J=4.8 Hz); MH+ 368; m.p 269-270; Calcd. for C₁₄H₁₁Cl₂N₅OS: C, 45.66; H, 3.01; N, 19.02. Found: C, 45.54; H, 2.70; N, 19.03.

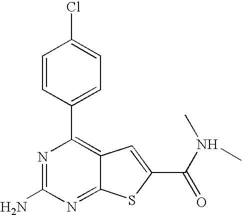

2-Amino-4-(4-chlorophenyl)-thieno[2,3-d]pyrimidine-6-carboxylic acid dimethyl amide (CDCl3) δ 7.80 (d, 2H, J=8.4 Hz), 7.50 (s, 1H), 7.50 (d, 2H, J=8.4 Hz), 5.31 (br s, 2H), 3.19 (br s, 6H); MH+ 333.

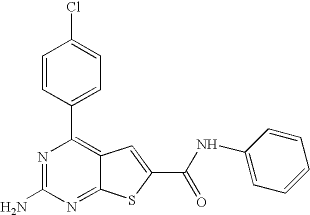

2-Amino-4-(4-chlorophenyl)-thieno[2,3-d]pyrimidine-6-carboxylic acid phenyl amide (CDCl₃/CD₃OD) δ 8.14 (s, 1H), 7.89 (d, 2H, J=8.8 Hz), 7.65 (d, 2H, J=8.8 Hz), 7.56 (d, 2H, J=8.4 Hz), 7.45 (s, 1H), 7.35 (t, 2H, J=8.4 Hz), 7.15 (t, 2H, J=8.4 Hz), 2.04 (s, 2H); MH+ 382.

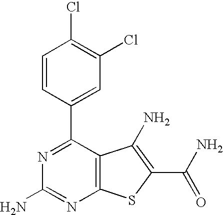

2,5-Diamino-4-(3,4-dichloro-phenyl)-thieno[2,3d]pyrimidine-6-carboxylic acid amide (DMSO-d6) δ 7.84 (m, 1H), 7.79 (m, 1H), 7.57 (m, 1H), 7.35 (br. s, 2H), 7.03 (br. s, 2H), 6.17 (br. s, 2H); MH+ 354.

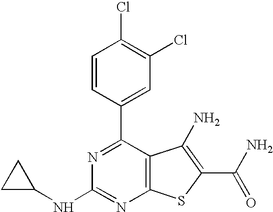

5-Amino-2-cyclopropylamino-4-(3,4-dichloro-phenyl)-thieno[2,3d]pyrimidine-6-carboxylic acid amide (DMSO-d6) δ 8.03 (br. s, 2H) 7.85 (br. s, 1H), 7.79 (app. d, 1H, J=8.1 Hz), 7.57 (m, 1H), 7.04 (br. s, 2H), 6.16 (br. s, 2H), 2.83 (m, 1H), 0.71 (m, 2H), 0.52 (m, 2H); MH+ 394.

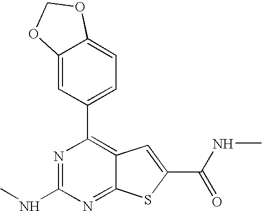

[4-(2H-benzo[3,4-d]1,3-dioxolan-5-yl)-2-(methylamino)thiopheno[2,3-d]pyrimidin-6-yl]-N-methylcarboxamide (CDCl3) δ 7.84 (s, 1H), 7.34-7.42 (m, 2H), 6.96 (dd, 2H, J1=8.0 Hz, J2=0.8 Hz), 6.07 (s, 2H), 3.10 (d, 3H, J=4.8 Hz), 2.96 (d, 3H, J=4.4 Hz); MH+ 343.

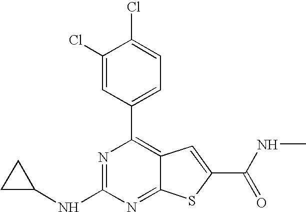

[4-(3,4-dichlorophenyl)-2-(cyclopropylamino)thiopheno[2,3-d]pyrimidin-6-yl]-N-methylcarboxamide (CDCl₃) δ 8.00 (s, 1H), 7.83 (s, 1H), 7.73 (d, 1H, J=6.4 Hz), 7.63 (d, 1H, J=8.4 Hz), 3.41 (s, 3H), 3.37 (m, 1H), 0.90 (d, 2H, J=5.2 Hz), 0.62 (s, 2H, J=2.8 Hz); MH+ 394.

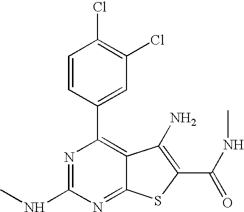

5-Amino-4-(3,4-dichloro-phenyl)-2-methylamino-thieno[2,3d]pyrimidine-6-carboxylic acid methylamide (DMSO-d6) δ 7.85 (br. s, 1H), 7.79 (m, 1H), 7.57 (m, 1H), 7.49 (m, 1H), 6.11 (br. s, 2H), 2.87 (d, 3H, J=3 Hz), 2.69 (d, 3H, J=4.5 Hz); MH+ 382; m.p. 230-230.5.

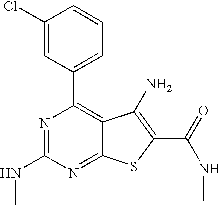

5-Amino-4-(3-chlorophenyl)-2-methylamino-thieno[2,3d]pyrimidine-6-carboxylic acid methylamide (DMSO-d6) δ 7.72 (bs, 1H), 7.63 (d, 2H), 7.57 (m, 2H), 7.45 (d, 1H), 5.96 (bs, 2H), 2.87 (d, 3H), 2.68 (d, 3H); MH+ 340; m.p.>240.

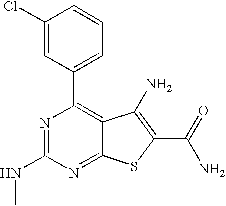

5-Amino-4-(3-chlorophenyl)-2-methylamino-thieno[2,3d]pyrimidine-6-carboxylic acid amide (DMSO-d6) δ 7.70 (bs, 1H), 7.63 (dd, 2H), 7.58 (m, 2H), 6.99 (s, 2H), 6.00 (bs, 2H), 2.87 (d, 3H); MH+ 334.

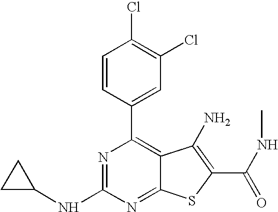

5-Amino-2-cyclopropyl-4-(3,4-dichloro-phenyl)-2-methylamino-thieno[2,3d]pyrimidine-6-carboxylic acid methylamide (DMSO-d6) δ 8.02 (br. s, 1H), 7.85 (m, 1H), 7.79 (m, 1H), 7.57 (m, 1H), 7.50 (m, 1H), 6.11 (br. s, 2H), 2.81 (m, 1H), 2.69 (d, 3H, J=4.5 Hz), 0.71 (m, 2H), 0.52 (m, 2H); MH+ 408; m.p. 233-233.5.

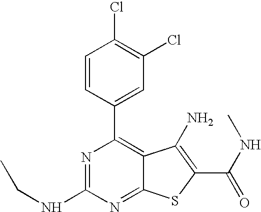

5-Amino-4-(3,4-dichloro-phenyl)-2-ethylamino-thieno[2,3d]pyrimidine-6-carboxylic acid methylamide (DMSO-d6) δ 7.84 (m, 1H), 7.79 (m, 1H), 7.56 (m, 1H), 7.48 (m, 1H), 6.10 (br. s, 2H), 3.33 (par. obs. q, 2H), 2.68 (d, 2H, J=4.5 Hz); 1.14 (t, 3H, J=7.1 Hz); MH+ 396; m.p. 220-221.

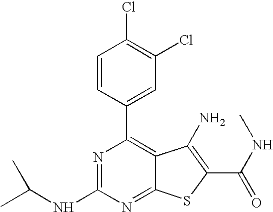

5-Amino-4-(3,4-dichloro-phenyl)-2-isopropylamino-thieno[2,3d]pyrimidine-6-carboxylic acid methylamide (DMSO-d6) δ 7.86 (m, 1H), 7.79 (m, 1H), 7.57 (m, 1H), 7.47 (m, 1H), 6.09 (br. s, 2H), 4.11 (m, 1H), 2.69 (d, 3H, J=4.5 Hz), 1.17 (d, 6H, J=6.3 Hz); MH+ 410; m.p. 198-199.

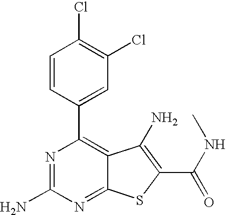

2,5-Diamino-4-(3,4-dichloro-phenyl)-thieno[2,3d]pyrimidine-6-carboxylic acid methylamide (DMSO-d6) δ 7.84 (d, 1H, J=2.1 Hz), 7.79 (d, 1H, J=8.4 Hz), 7.56 (dd, 1H, J=2.0, 8.3 Hz), 7.50 (dd, 1H, J=3.0, 9.0 Hz), 7.25 (br. s, 2H), 6.10 (br. s, 2H), 2.68 (d, 3H, J=4.2 Hz); MH+ 368; m.p. 298.

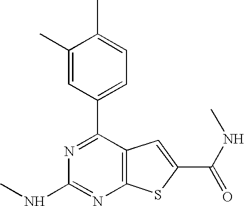

[4-(3,4-dimethylphenyl)-2-(methylamino)thiopheno[2,3-d]pyrimidin-6-yl]-N-methylcarboxamide (DMSO, 400 MHz) δ 8.63 (d, 2H, J=3.6 Hz), 8.00 (s, 1H), 7.63 (m, 3H), 2.90 (d, 3H, J=4.0 Hz), 2.76 (d, 3H, J=4.4 Hz), 2.35 (s, 3H), 2.34 (s, 3H); MH+ 327; Calcd for C₁₇H₁₈N₄OS: C, 62.55; H, 5.56; N, 17.16. Found: C, 62.38; H, 5.25; N, 16.82.

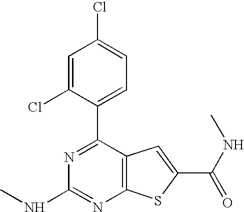

[4-(2,4-dichlorophenyl)-2-(methylamino)thiopheno[2,3-d]pyrimidin-6-yl]-N-methylcarboxamide (DMSO) δ 8.50 (m, 1H), 7.88 (d, 1H, J=2.4 Hz), 7.78 (br s, 1H), 7.64 (q, 2H, J=8.4 Hz), 7.52 (s, 1H), 2.88 (d, 3H, J=4.8 Hz), 2.72 (d, 3H, J=4.8 Hz), 2.35 (s, 3H), 2.34 (s, 3H); MH+ 367; Calcd. for C₁₅H₁₂Cl₂N₄OS: C, 49.06; H, 3.29; N, 15.26. Found: C, 49.02; H, 3.90; N, 15.19.

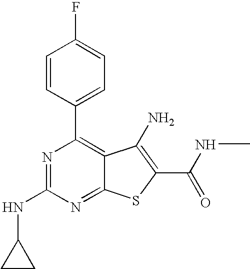

5-Amino-2-cyclopropylamino 4-(4-fluoro-phenyl)-thieno[2,3d]pyrimidine-6-carboxylic acid methylamide (DMSO-d6) δ 7.96 (br.s, 1H), 7.64 (m, 2H), 7.49 (m, 1H), 7.34 (m, 2H), 5.97 (br. s, 2H), 2.83 (m, 1H), 2.69 (d, 3H, J=3.6 Hz), 0.71 (m, 2H), 0.52 (m, 2H); MH+ 358; m.p. 162-163.

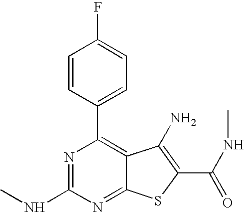

5-Amino-4-(4-fluoro-phenyl) 2-methylamino-thieno[2,3d]pyrimidine-6-carboxylic acid methylamide (DMSO-d6) δ 7.62 (m, 3H), 7.46 (m, 1H), 7.38 (m, 2H), 5.96 (br. s, 2H), 2.87 (d, 3H, J=5.1 Hz), 2.68 (d, 3H, J=4.2 Hz); MH+ 332; m.p. 269.

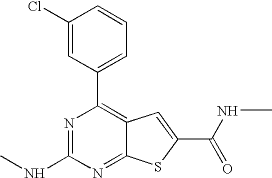

[4-(3-chlorophenyl)-2-(methylamino)thiopheno[2,3-d]pyrimidin-6-yl]-N-methylcarboxamide (DMSO) δ 8.69 (d, 1H, J=4.0 Hz), 8.01 (s, 1H), 7.85 (m, 2H), 7.75 (br s, 1H), 7.64 (m, 2H), 2.90 (d, 3H, J=5.6 Hz), 2.77 (d, 3H, J=6.0 Hz); MH+ 333; Calcd. for C₁₅H₁₃ClN₄OS: C, 54.13; H, 3.94; N, 16.83. Found: C, 54.40; H, 3.77; N, 16.60.

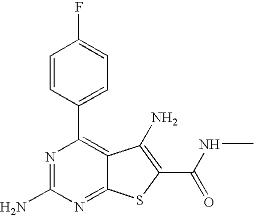

2,5-Diamino-4-(4-fluoro-phenyl)-thieno[2,3d]pyrimidine-6-carboxylic acid methyl amide (DMSO-d6) δ 7.62 (m, 2H), 7.47 (m, 1H), 7.38 (m, 2H), 7.20 (br. s, 2H), 5.97 (br. s, 2H), 2.67 (d, 3H, J=4.5 Hz); MH+ 318; m.p. 282.

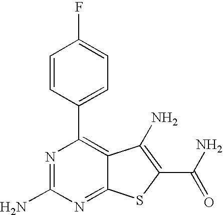

2,5-Diamino-4-(4-fluoro-phenyl)-thieno[2,3d]pyrimidine-6-carboxylic acid amide (DMSO-d6) δ 7.63 (m, 2H), 7.38 (m, 2H), 7.20 (br. s, 2H), 6.99 (br. s 2H), 6.03 (br. s, 2H); MH+ 304; m.p. 267-268.

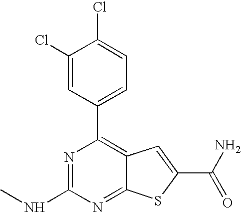

[4-(3,4-dichlorophenyl)-2-(methylamino)thiopheno[2,3-d]pyrimidin-6-yl]-carboxylic acid amide (DMSO) δ 8.21 (br s, 1H), 8.09 (br s, 1H), 8.07 (s, 1H), 7.87 (s, 2H), 7.84 (br s, 1H), 7.56 (s, 1H), 2.90 (m, 3H); MH+ 353.

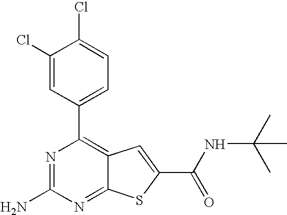

2-Amino-4-(3,4-dichlorophenyl)-thieno[2,3-d]pyrimidine-6-carboxylic acid tert-butylamide (CD₂Cl₂) δ 7.97 (d, 1H, J=2.0 Hz), 7.70 (dd, 1H, J1=8.4 Hz, J2=2.0 Hz), 7.62 (d, 1H, J=8.4 Hz), 7.54 (s, 1H), 5.86 (br s, 1H), 5.32 (br s, 2H), 1.42 (s, 9H); MH+ 395; Calcd. for C₁₇H₁₆Cl₂N₄OS: C, 51.65; H, 4.08; N, 14.07. Found: C, 51.81; H, 3.64; N, 13.77.

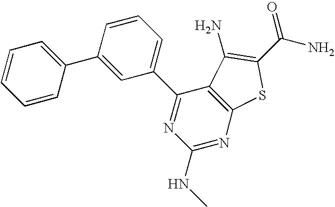

5-Amino-4(-3-phenylphenyl)-2-methylamino-thieno[2,3-d]pyrimidine-6-carboxylic acid amide (DMSO-d6) δ 7.87 (m, 2H), 7.74 (d, 2H), 7.68 (t, 2H), 7.55 (bs, 1H), 7.5 (t, 2H), 7.41 (t, 1H), 6.99 (bs, 2H), 6.05 (bs, 2H), 2.88 (d, 3H); MH+ 376; m.p. 230-231.

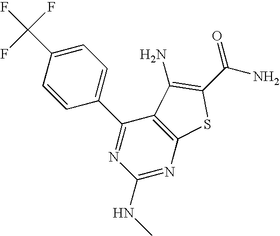

5-Amino-4(4-trifluoromethyl-phenyl)-2-methylamino-thieno[2,3-d]pyrimidine-6-carboxylic acid amide (DMSO-d6) δ 7.89 (d, 2H), 7.81 (bd, 3H), 7.0 (s, 2H), 6.0 (bs, 2H), 2.87 (d, 3H); MH+ 368; m.p. 233-234.

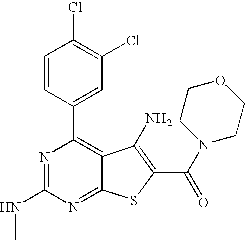

[5-Amino-4-(3,4-dichloro-phenyl)-2-methylamino-thieno[2,3-d]pyrimidin-6-yl]-morpholin-4-yl-methanone (DMSO-d6) δ 7.87 (br. s, 1H) 7.80 (m, 1H), 7.60 (m, 2H), 5.50 (br. s, 2H), 3.58 (M, 8H), 2.86 (d, 3H, J=4.5 Hz); MH+ 434; m.p. 205.

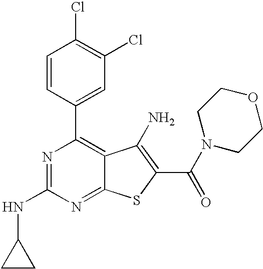

[5-Amino-2-cyclopropylamino-4-(3,4-dichloro-phenyl)-thieno[2,3-d]pyrimidin-6-yl]-morpholin-4-yl-methanone (DMSO-d6) δ 8.05 (br. s, 1H), 7.88 (br. s, 1H), 7.80 (app. d, 1H, J=8.1 Hz), 7.60 (m, 1H), 5.49 (br. s, 2H), 3.59 (m, 8H), 2.8 (m, 1H), 0.71 (m, 2H), 0.51 (m, 2H); MH+ 464; m.p. 168-168.5.

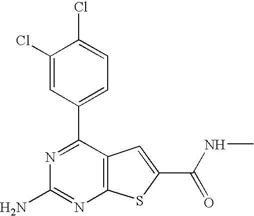

4-(3,4-Dichlorophenyl)-2-amino-thieno[2,3-d]pyrimidine-6-carboxylic acid methyl amide (DMSO) δ 8.20 (br s, 1H), 8.67 (d, 1H, J=3.9 Hz), 8.09 (s, 1H), 8.00 (s, 1H), 7.86 (s, 2H), 7.25 (s, 2H), 2.77 (d, 3H, J=3.6 Hz); MH+ 353; Calcd. for C₁₄H₁₀Cl₂N₄OS.H2O: C, 45.29; H, 3.26; N, 15.09. Found: C, 45.63; H, 3.21; N, 14.60.

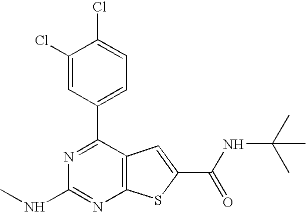

4-(3,4-Dichlorophenyl)-2-methylamino-thieno[2,3-d]pyrimidine-6-carboxylic acid tert-butylamide (CDCl3) δ 7.97 (br s, 1H), 7.69 (d, 1H, J=10.2 Hz), 7.62 (s, 1H), 7.59 (m, 2H), 5.73 (br s, 1H), 5.37 (br s, 1H), 3.12 (d, 3H, J=4.8 Hz), 1.48 (s, 9H); MH+ 409.

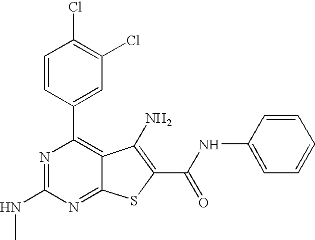

5-Amino-4-(3,4-dichloro-phenyl)-2-methylamino-thieno[2,3-d]pyrimidine-6-carboxylic acid phenylamide (DMSO-d6) δ 9.29 (br. s, 1H), 7.88 (br. s, 1H), 7.82 (m, 2H), 7.62 (m, 3H), 7.30 (m, 2), 7.05 (m, 1H), 6.31 (br, s, 2H), 2.89 (d, 3H, J=4.8 Hz); MH+ 446; m.p. 245-246.

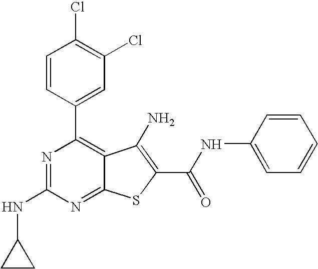

5-Amino-2-cyclopropylamino-4-(3,4-dichloro-phenyl)-thieno[2,3-d]pyrimidine-6-carboxylic acid phenylamide (DMSO-d6) δ 9.29 (br. s, 1H), 8.11 (br. s, 1H), 7.89 (br. s, 1H), 7.81 (m, 1H), 7.62 (m, 3H), 7.31 (m, 2H), 7.10 (m, 1H), 6.32 (br. s, 2H), 2.85 (m, 1H), 0.73 (m, 2H), 0.54 (m, 2H); MH+ 472; m.p. 216-217.

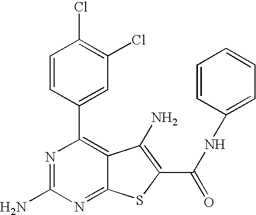

2,5-Diamino-4-(3,4-dichloro-phenyl)-thieno[2,3-d]pyrimidine-6-carboxylic acid phenylamide (DMSO-d6) δ 9.30 (br. s, 1H), 7.87 (br. s, 1H), 7.81 (m, 1H), 7.62 (m, 3H), 7.36 (par. obs. br. s, 2H), 7.30 (m, 2H), 7.05 (m, 1H), 6.34 (br. s, 2H); MH+ 430; m.p. 252-253.

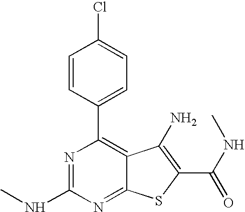

5-Amino-4-(4-dichloro-phenyl)-2-methylamino-thieno[2,3-d]pyrimidine-6-carboxylic acid methylamide (DMSO-d6) δ 7.73 (br. s, 1H), 7.61 (br. s, 4H), 7.46 (m, 1H), 5.98 (br. s, 2H), 2.87 (d, 3H, J=4.8 Hz), 2.68 (d, 3H, J=3.9 Hz); MH+ 348; m.p. 261-262.

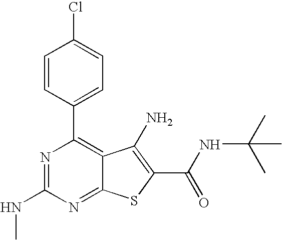

5-Amino-4-(4-dichloro-phenyl)-2-methylamino-thieno[2,3-d]pyrimidine-6-carboxylic acid tert-butylamide (DMSO-d6) δ 7.70 (br. s, 1H), 7.61 (br. s, 4H), 6.53 (br. s, 1H), 5.92 (br. s, 2H), 2.87 (d, 3H, J=4.5 Hz), 1.34 (s, 9H); MH+ 390; m.p. 280-281.

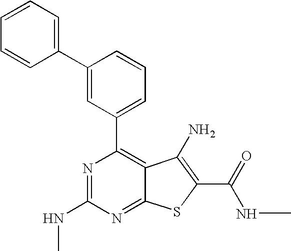

5-Amino-4(3-phenylphenyl)-2-methylamino-thieno[2,3-d]pyrimidine-6-carboxylic acid methylamide (DMSO-d6) δ 7.87 (dd, 1H), 7.84 (bs, 1H), 7.74 (d, 2H), 7.65 (t, 2H), 7.58 (bs, 1H), 7.49 (t, 2H), 7.46 (d, 1H), 7.42 (t, 1H), 5.99 (bs, 2H), 2.88 (d, 3H), 2.68 (d, 3H); MH+ 390; m.p. 99-100; Calcd. for C₂₁H₁₉N₅OS: C, 64.46; H, 4.95; N, 17.90. Found: C, 64.28; H, 5.01; N, 17.64.

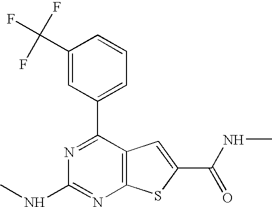

N-methyl{2-(methylamino)-4-[3-(trifluoromethyl)phenyl]thiopheno[2,3-d]pyrimidin-6-yl}carboxamide (DMSO) δ 8.65 (d, 1H, J=4.8 Hz), 8.19 (m, 2H), 7.98 (d, 2H, J=5.2 Hz), 7.86 (t, 1H, J=7.6 Hz), 7.79 (br s, 1H), 2.91 (d, 3H, J=4.4 Hz), 2.77 (d, 3H, J=4.4 Hz); MH+ 367; Calcd. for C₁₆H₁₃F₃N₄OS. 0.5H₂O: C, 50.59; H, 3.85; N, 14.75. Found: C, 50.41; H, 3.18; N, 14.45.

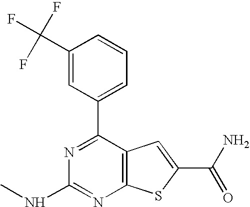

2-Methylamino-4-(3-trifluoromethylphenyl)-thieno[2,3-d]pyrimidine carboxylic acid amide (DMSO) δ 8.19 (m, 3H), 8.05 (s, 1H), 7.98 (d, 1H, J=7.6 Hz), 7.85 (t, 1H, J=8.0 Hz), 7.79 (br s, 1H), 7.53 (br s, 1H), 2.91 (d, 3H, J=3.6 Hz); MH+ 353.

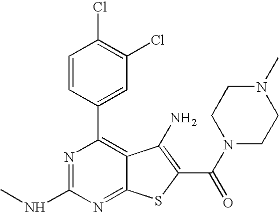

[5-Amino-4-(3,4-dichloro-phenyl)-2-methylamino-thieno[2,3-d]pyrimidin-6-yl]-(4-methyl-piperazine-1-yl)-methanone (DMSO-d6) δ 7.86 (br. s, 1H), 7.80 (app. d, 2H, J=8.7 Hz), 7.60 (m, 1H), 5.46 (br. s, 2H), 3.55 (m, 4H), 2.86 (d, 3H, J=4.8 Hz), 2.33 (m, 4H), 2.19 (s, 3H); MH+ 451.

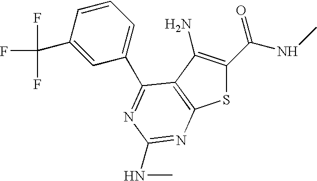

5-Amino-4-(3-trifluoromethylphenyl)-2-methylamino-thieno[2,3-d]pyrimidine-6-carboxylic acid methylamide (DMSO-d6) δ 8.02 (bs, 1H), 8.0 (bs, 1H), 7.95 (s, 1H), 7.85 (m, 2H), 7.58 (m, 1H), 6.05 (bs, 2H), 2.96 (d, 3H), 2.77 (d, 3H); MH+ 382; m.p. 215-216.

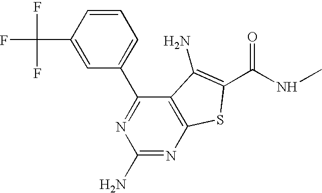

5-Amino-4-(3-trifluoromethylphenyl)-2-amino-thieno[2,3-d]pyrimidine-6-carboxylic acid methylamide (DMSO-d6) δ 8.02 (br s, 1H), 7.97 (m, 2H), 7.85 (t, 1H), 7.60 (m, 1H), 7.34 (bs, 1H), 6.06 (br s, 2H), 2.67 (d, 3H); MH+ 368; m.p.>240.

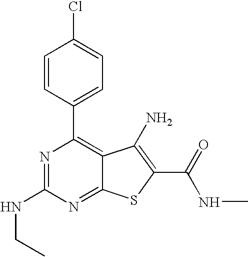

5-Amino-4-(4-chloro-phenyl)-2-ethylamino-thieno[2,3-d]pyrimidine-6-carboxylic acid methylamide (DMSO-d6) δ 7.80 (br. s, 1H), 7.61 (br. s, 4H), 7.46 (m, 1H), 5.97 (br. s, 2H), 3.40 (par. obs. q, 2H), 2.68 (d, 3H, J=4.2 Hz), 1.14 (t, 3H, J=7.1 Hz); MH+ 362.

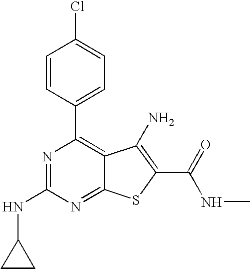

5-Amino-4-(4-chloro-phenyl)-2-ethylamino-thieno[2,3-d]pyrimidine-6-carboxylic acid methylamide (DMSO-d6) δ 7.97 (br. s, 1H), 7.61 (br. s, 4H), 7.48 (m, 1H), 5.98 (br. s, 2H), 2.81 (m, 1H), 2.69 (d, 3H, J=4.5 Hz), 0.70 (m, 2H), 0.52 (m, 2H); MH+ 374.

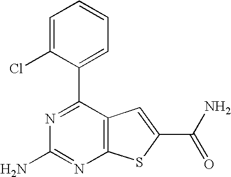

4-(2-chlorophenyl)-2-amino-thieno[2,3-d]pyrimidine-6-carboxylic acid amide (DMSO) δ 8.08 (br s, 1H), 7.66 (dt, 1H, J1=8.0 Hz, J2=0.8 Hz), 7.57 (m, 4H), 7.44 (br s, 1H), 7.21 (br s, 2H); MH+ 305.

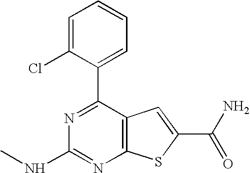

[4-(3,4-dichlorophenyl)-2-(methylamino)thiopheno[2,3-d]pyrimidin-6-yl]-carboxylic acid amide (DMSO) δ 8.08 (br s, 1H), 7.73 (br s, 1H), 7.66 (d, 1H, J=7.6 Hz), 7.56 (m, 4H), 7.44 (br s, 1H), 2.89 (d, 3H, J=4.8 Hz); MH+ 319.

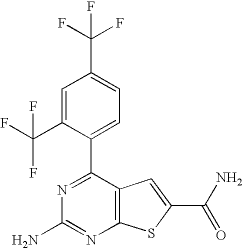

2-Amino-{4-[2,4-Bis(trifluoromethyl)phenyl]-thieno[2,3-d]pyrimidine-6-carboxylic acid amide (DMSO) δ 8.31 (s, 1H), 8.27 (d, 1H, J=8.4 Hz), 7.96 (br s, 1H), 7.93 (d, 1H, J=8.4 Hz), 7.51 (s, 1H), 7.46 (br s, 1H), 7.27 (br s, 2H); MH+ 407; Calcd for C₁₆H₁₀F₆N₄OS: C, 45.72; H, 2.40; N, 13.33. Found: C, 45.82; H, 2.40; N, 13.21.

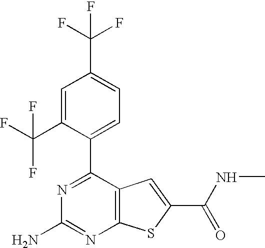

2-Amino-{4-[2,4-Bis(trifluoromethyl)phenyl]-thieno[2,3-d]pyrimidine-6-carboxylic acid methylamide (DMSO) δ 8.46 (d, 1H, J=4.4 Hz), 8.32 (s, 1H), 8.28 (d, 1H, J=8.4 Hz), 7.93 (d, 1H, J=8.4 Hz), 7.43 (s, 1H), 7.29 (br s, 2H), 2.70 (d, 3H, J=4.8 Hz); MH+ 421.

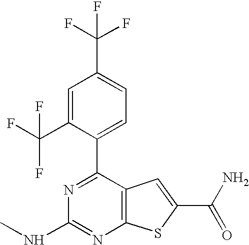

{4-[2,4-Bis(trifluoromethyl)phenyl]-2-(methylamino)thiopheno[2,3-d]pyrimidin-6-yl]-carboxamide (DMSO) δ 8.31 (s, 1H), 8.29 (d, 1H, J=8.4 Hz), 7.91 (m, 2H), 7.79 (m, 1H), 7.52 (m, 1H), 7.48 (br s, 1H), 2.88 (br s, 3H); MH+ 421.

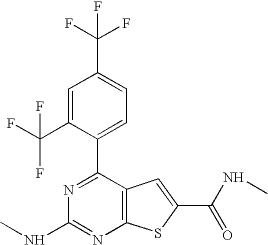

{4-[2,4-Bis(trifluoromethyl)phenyl]-2-(methylamino)thiopheno[2,3-d]pyrimidin-6-yl]-N-methylcarboxamide (DMSO, 400 MHz) δ 8.47 (br s, 1H), 8.32 (s, 1H), 8.29 (d, 2H, J=8.4 Hz), 7.95 (m, 1H), 7.79 (br s, 1H), 7.45 (br s, 1H), 2.89 (br s, 3H), 2.71 (d, 3H, J=4.4 Hz); MH+ 435.

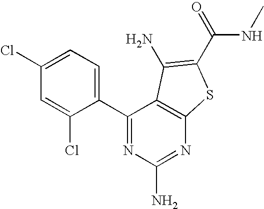

5-Amino-4-(2,4-dichlorophenyl)-2-amino-thieno[2,3-d]pyrimidine-6-carboxylic acid methylamide (DMSO) δ 7.82 (d, 1H), 7.59 (m, 2H), 7.48 (q, 1H), 7.27 (bs, 2H), 5.71 (bs, 2H), 2.66 (d, 3H); MH+ 368/370; m.p. 153-155.

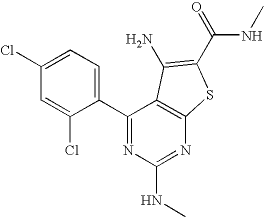

5-Amino-4-(2,4-dichlorophenyl)-2-methylamino-thieno[2,3-d]pyrimidine-6-carboxylic acid methylamide (DMSO) δ 7.83 (d, 1H), 7.8 (bs, 1H), 7.6 (m, 1H), 7.58 (d, 1H), 7.46 (q, 1H), 5.71 (bs, 2H), 2.86 (bs, 3H), 2.50 (m, 3H); MH+ 382/384; m.p.>240.

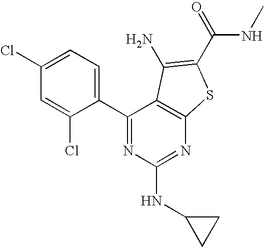

5-Amino-4-(2,4-dichlorophenyl)-2-cyclopropylamino-thieno[2,3-d]pyrimidine-6-carboxylic acid methylamide (DMSO) δ 8.05 (bs, 1H), 7.82 (d, if H), 7.58 (m, 2H), 7.48 (q, 1H), 5.72 (bs, 2H), 2.81 (m, 1H), 2.68 (d, 3H), 0.72 (m, 2H), 0.5 (m, 2H); MH+ 408/410; m.p. 213-215.

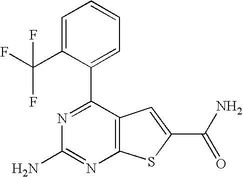

2-(Amino)-4-[2-(trifluoromethyl)phenyl]thiopheno[2,3-d]pyrimidine-6-carboxylic acid amide (DMSO) δ 8.03 (br s, 1H), 7.95 (d, 1H, J=7.2 Hz), 7.85 (t, 1H, J=6.8 Hz), 7.78 (t, if H, J=7.2 Hz), 7.62 (d, 1H, J=7.6 Hz), 7.49 (s, 1H), 7.44 (br s, 1H), 7.20 (br s, 2H); MH+ 339.

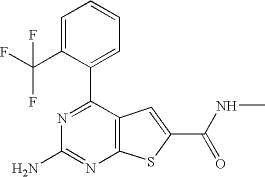

2-(Amino)-4-[2-(trifluoromethyl)phenyl]thiopheno[2,3-d]pyrimidine-6-carboxylic acid methylamide (DMSO) δ 8.02 (br s, 1H), 7.95 (d, 1H, J=7.6 Hz), 7.84 (t, 1H, J=7.6 Hz), 7.79 (d, 1H, J=7.6 Hz), 7.68 (br s, 1H), 7.64 (br s, 1H), 7.51 (br s, 1H), 7.44 (br s, 1H), 2.88 (br s, 3H); MH+ 353.

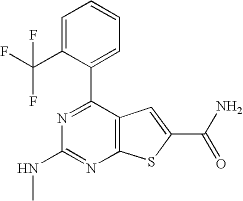

2-(Methylamino)-4-[2-(trifluoromethyl)phenyl]thiopheno[2,3-d]pyrimidine-6-carboxylic acid amide (DMSO) δ 8.03 (br s, 1H), 7.95 (d, 1H, J=8.0 Hz), 7.84 (d, 1H, J=8.0 Hz), 7.79 (d, 1H, J=7.2 Hz), 7.70 (br s, 1H), 7.64 (br s, 1H), 7.50 (br s, 1H), 7.44 (br s, 1H), 2.87 (br s, 3H); MH+ 353.

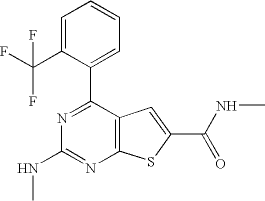

N-methyl{2-(methylamino)-4-[2-(trifluoromethyl)phenyl]thiopheno[2,3-d]pyrimidin-6-yl}carboxamide (DMSO) δ 8.47 (br s, 1H), 8.32 (s, 1H), 8.29 (d, 2H, J=8.4 Hz), 7.95 (m, 1H), 7.79 (br s, 1H), 7.45 (br s, 1H), 2.89 (br s, 3H), 2.71 (d, 3H, J=4.4 Hz); MH+ 367.

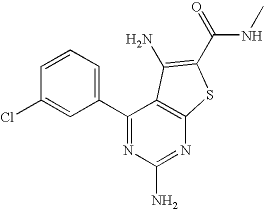

5-Amino-4-(3-chlorophenyl)-2-amino-thieno[2,3-d]pyrimidine-6-carboxylic acid methylamide (DMSO) δ 7.72 (m, 2H), 7.64 (m, 2H), 7.58 (m, 1H), 7.31 (bs, 2H), 6.05 (bs, 2H), 2.76 (d, 3H); MH+ 334; m.p.>250.

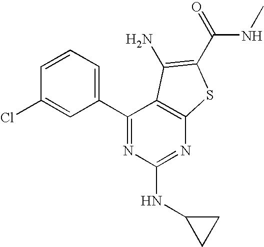

5-Amino-4-(3-chlorophenyl)-2-cyclopropylamino-thieno[2,3-d]pyrimidine-6-carboxylic acid methylamide (DMSO) d 7.99 (bs, 1H), 7.65 (bs, 1H), 7.55 (m, 2H), 7.50 (m, 2H), 5.98 (bs, 2H), 2.81 (m, 1H), 2.68 (d, 3H), 0.71 (d, 2H), 0.52 (m, 2H); MH+ 374; m.p. 225-227.

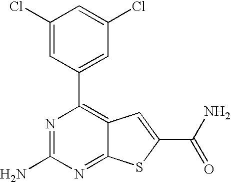

2-Amino-4-(3,5-dichlorophenyl)-thieno[2,3-d]pyrimidine-6-carboxylic acid amide (CDCl3/CD3OD) δ 7.93 (s, 1H), 7.78 (s, 1H), 7.77 (s, 1H), 7.57 (t, 1H, J=2.0 Hz), 6.98 (s, 1H); MH+ 339.

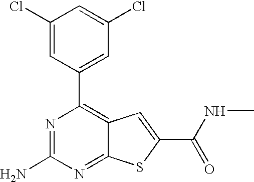

2-Amino-4-(3,5-dichlorophenyl)-thieno[2,3-d]pyrimidine-6-carboxylic acid methylamide (DMSO) δ 8.73 (q, 1H, J=4.8 Hz), 7.99 (s, 1H), 7.89 (t, 1H, J=2.0 Hz), 7.86 (t, 1H, J=1.2 Hz), 7.26 (br s, 2H), 2.79 (d, 3H, J=4.8 Hz); MH+ 353.

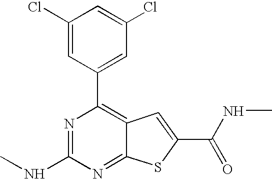

[4-(3,5-dichlorophenyl)-2-(methylamino)thiopheno[2,3-d]pyrimidin-6-yl]-N-methylcarboxamide (DMSO) δ 8.72 (m, 1H), 7.99 (s, 1H), 7.89 (m, 2H), 2.89 (br s, 3H), 2.78 (d, 3H, J=4.8 Hz); MH+ 367.

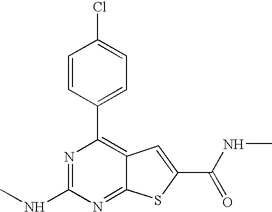

[4-(4-chlorophenyl)-2-(methylamino)thiopheno[2,3-d]pyrimidin-6-yl]-N-methylcarboxamide (CDCl₃) δ 7.80 (d, 2H, J=8.4 Hz), 7.71 (s, 1H), 7.52 (d, 2H, J=8.8 Hz), 5.92 (br s, 1H), 5.37 (br s, 1H), 3.12 (d, 3H, J=4.8 Hz), 3.02 (d, 3H, J=4.8 Hz); MH+ 333; Calcd. for C₁₅H₁₃ClN₄OS.0.75H₂O: C, 52.02; H, 4.22; N, 16.24. Found: C, 51.84; H, 4.00; N, 15.98.

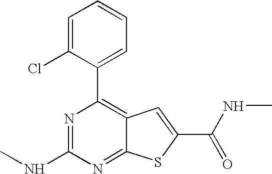

[4-(2-chlorophenyl)-2-(methylamino)thiopheno[2,3-d]pyrimidin-6-yl]-N-methylcarboxamide (DMSO) δ 8.56 (m, 1H), 7.76 (br s, 1H), 7.67 (d, 2H, J=8.4 Hz), 7.55 (m, 4H), 2.90 (d, 3H, J=4.8 Hz), 2.71 (d, 3H, J=4.4 Hz); MH+ 333.

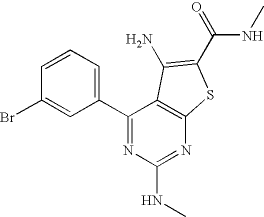

5-Amino-4-(3-bromophenyl)-2-methylamino-thieno[2,3-d]pyrimidine-6-carboxylic acid methylamide (DMSO) δ 7.77 (d, 2H), 7.72 (bs, 1H), 7.57 (m, 1H), 7.47 (m, 2H), 5.96 (bs, 2H), 2.87 (d, 3H), 2.68 (d, 3H); MH+ 392/394; m.p. 245.

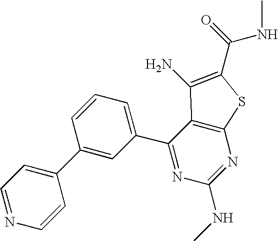

5-Amino-4-(3-(4-pyridyl))-2-methylamino-thieno[2,3-d]pyrimidine-6-carboxylic acid methylamide (DMSO) δ 8.67 (d, 2H), 8.00 (m, 2H), 7.78 (d, 2H), 7.70 (m, 3H), 7.46 (q, 1H), 5.97 (bs, 2H), 2.88 (d, 3H), 2.68 (d, 3H); MH+ 391; m.p. 132-133.

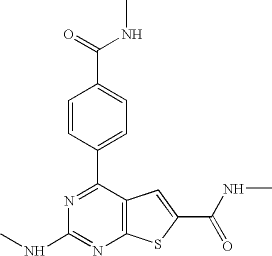

2-Methylamino-4-(4-methylcarbamoylphenyl)-thieno[2,3-d]pyrimidine-6-carboxylic acid methylamide (DMSO) δ 8.17 (m, 1H), 8.09 (s, 1H), 8.07 (m, 1H), 8.00 (m, 2H), 2.91 (m, 3H), 2.83 (d, 3H, J=4.4 Hz), 2.76 (d, 3H, J=4.8 Hz); MH+ 356.

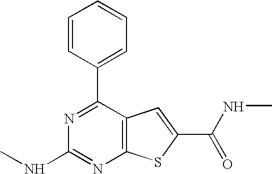

2-Methylamino-4-phenyl-thieno[2,3-d]pyrimidine-6-carboxylic acid methylamide (DMSO) δ 8.61 (d, 1H, J=4.4 Hz), 7.96 (s, 1H), 7.82 (br s, 2H), 7.55 (m, 4H), 2.84 (br s, 3H), 2.71 (d, 3H, J=4.4 Hz); MH+ 299.

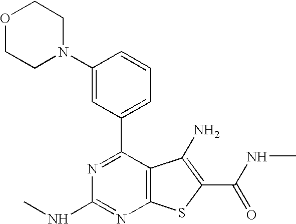

5-Amino-2-methylamino-4-(3-morpholin-4-yl-phenyl)-thieno[2,3-d]pyrimidine-6-carboxylic acid methyl amide (CDCl3) δ 7.42 (t, 1H, J=8.0 Hz), 6.98-7.06 (m, 3H), 6.01 (br s, 2H), 5.37 (br s, 1H), 5.26 (m, 1H), 3.86 (t, 4H, J=5.2 Hz), 3.22 (t, 4H, J=4.8 Hz), 3.09 (d, 3H, J=5.2 Hz), 2.93 (d, 3H, J=5.2 Hz); MH+ 399.

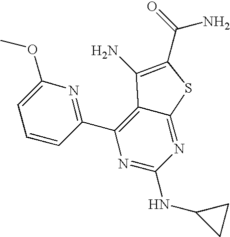

5-amino-2-(cyclopropylamino)-4-(6-methoxy(2-pyridyl))thiopheno[2,3-d]pyrimidine-6-carboxamide ¹H-NMR (400 MHz, DMSO-d6) δ ppm 7.99-7.96 (m, 2H) 7.65 (bs, 1H) 7.32 (bs, 2H) 7.07 (d, 1H) 6.99 (bs, 2H) 3.92 (s, 3H) 2.82 (m, 1H) 0.74-0.70 (m, 2H) 0.55-0.53 (m, 2H). MS m/z calculated for (M+H)⁺ 357, found 357.

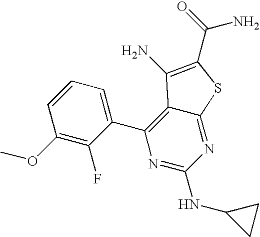

5-amino-2-(cyclopropylamino)-4-(2-fluoro-3-methoxyphenyl)thiopheno[2,3-d]pyrimidine-6-carboxamide ¹H-NMR (300 MHz, DMSO-d6) δ ppm 8.03 (bs, 1H) 7.39-7.28 (m, 2H) 7.04 (s, 1H) 7.02 (bs, 2H) 5.83 (bs, 2H) 3.9 (s, 3H) 2.81-2.77 (m, 1H) 0.719 (bs, 2H) 0.52 (bs, 2H). MS m/z calculated for (M+H)⁺ 374, found 374.

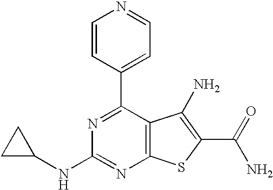

5-amino-2-(cyclopropylamino)-4-(4-pyridyl)thiopheno[2,3-d]pyrimidine-6-carboxamide ¹H NMR (400 MHz, DMSO-d6): δ ppm 0.51-0.54 (m, 2H) 0.70-0.72 (m, 2H) 2.80-2.83 (m, 1H) 6.00 (s, 2H) 7.05 (s, 2H) 7.58 (s, 2H) 8.06 (s, 1H) 8.76 (s, 2H). MS m/z calculated for (M+H)⁺ 327, found 327. Analytical HPLC retention time: 3.46 minutes

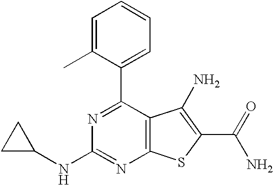

5-amino-2-(cyclopropylamino)-4-(2-methylphenyl)thiopheno[2,3-d]pyrimidine-6-carboxamide ¹H NMR (400 MHz, DMSO-d₆): δ ppm 0.50-0.54 (m, 2H) 0.70-0.72 (m, 2H) 2.11 (s, 3H) 2.80-2.83 (m, 1H) 5.64 (s, 2H) 6.97 (s, 2H) 7.27-7.47 (m, 4H) 7.98 (s, 1H). MS m/z calculated for (M+H)⁺ 340, found 340. Analytical HPLC retention time: 4.97 minutes

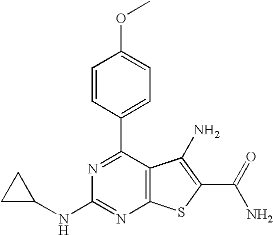

5-amino-2-(cyclopropylamino)-4-(4-methoxyphenyl)thiopheno[2,3-d]pyrimidine-6-carboxamide ¹H NMR (400 MHz, DMSO-d6): δ ppm 0.50-0.54 (m, 2H) 0.70-0.72 (m, 2H) 2.80-2.82 (m, 1H) 3.8 (s, 3H) 6.00 (s, 2H) 6.99 (s, 2H) 7.09-7.11 (d, J=8 Hz, 2H) 7.53-7.55 (d, J=8 Hz, 2H) 7.87 (s, 1H). MS m/z calculated for (M+H)⁺ 356, found 356. Analytical HPLC retention time: 4.91 minutes

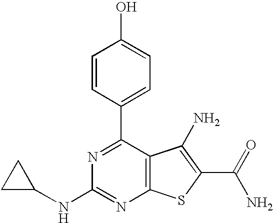

5-amino-2-(cyclopropylamino)-4-(4-hydroxyphenyl)thiopheno[2,3-d]pyrimidine-6-carboxamide ¹H NMR (400 MHz, DMSO-d6): δ ppm 0.49-0.53 (m, 2H) 0.69-0.72 (m, 2H) 2.80-2.82 (m, 1H) 6.09 (s, 2H) 6.8-6.94 (m, 4H) 7.41-7.43 (d, J=8 Hz, 2H) 7.84 (s, 1H) 9.95 (s, 1H). MS m/z calculated for (M+H)⁺ 342, found 341.9. Analytical HPLC retention time: 4.3 minutes

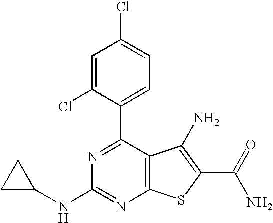

5-amino-4-(2,4-dichlorophenyl)-2-(cyclopropylamino)thiopheno[2,3-d]pyrimidine-6-carboxamide ¹H NMR (400 MHz, DMSO-d₆): δ ppm 0.50-0.52 (m, 2H) 0.69-0.72 (m, 2H) 2.79-2.80 (m, 1H) 5.77 (s, 2H) 7.0 (s, 2H) 7.55-7.59 (m, 2H) 7.84 (s, 1H) 8.09 (s, 1H). MS m/z calculated for (M+H)⁺ 395, found 395. Analytical HPLC retention time: 5.68 minutes

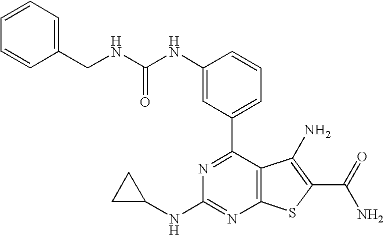

5-amino-2-(cyclopropylamino)-4-(3-{[benzylamino]carbonylamino}phenyl)thiopheno[2,3-d]pyrimidine-6-carboxamide ¹H NMR (400 MHz, DMSO-d6): δ ppm 0.50-0.54 (m, 2H) 0.70-0.72 (m, 2H) 2.80-2.83 (m, 1H) 4.29-4.31 (d, J=8 Hz, 2H) 6.07 (s, 2H) 6.69-6.72 (t, 1H) 6.97 (s, 2H) 7.06-7.08 (d, J=8 Hz, 1H) 7.22-7.42 (m, 8H) 7.52-7.54 (d, J=8 Hz, 1H) 7.65 (s, 1H) 7.92 (s, 1H) 8.8 (s, 1H). MS m/z calculated for (M+H)⁺ 474, found 474. Analytical HPLC retention time: 5.09 minutes

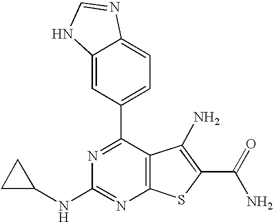

5-amino-4-benzimidazol-6-yl-2-(cyclopropylamino)thiopheno[2,3-d]pyrimidine-6-carboxamide ¹H NMR (400 MHz, DMSO-d₆): δ ppm 0.50-0.54 (m, 2H) 0.70-0.72 (m, 2H) 2.80-2.83 (m, 1H) 6.05 (s, 2H) 6.96 (s, 2H) 7.34-7.36 (d, J=8 Hz, 1H) 7.73-7.75 (d, J=8 Hz, 1H) 7.80 (s, 1H) 7.91 (s, 1H) 8.18 (s, 1H). MS m/z calculated for (M+H)⁺ 366, found 366. Analytical HPLC retention time: 3.02 minutes

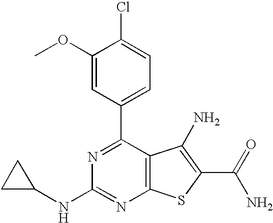

5-amino-4-(4-chloro-3-methoxyphenyl)-2-(cyclopropylamino)thiopheno[2,3-d]pyrimidine-6-carboxamide ¹H NMR (400 MHz, DMSO-d₆): δ ppm 0.50-0.54 (m, 2H) 0.70-0.72 (m, 2H) 2.80-2.83 (m, 1H) 3.89 (s, 3H) 6.09 (s, 2H) 7.00 (s, 2H) 7.16 (s, 3H) 7.34 (s, 1H) 7.58-7.60 (d, J=8 Hz, 1H) 8.00 (s, 1H). MS m/z calculated for (M+H)⁺ 391, found 391. Analytical HPLC retention time: 5.36 minutes

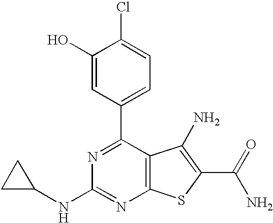

5-amino-4-(4-chloro-3-hydroxyphenyl)-2-(cyclopropylamino)thiopheno[2,3-d]pyrimidine-6-carboxamide ¹H NMR (300 MHz, DMSO-d₆): δ ppm 0.50-0.54 (m, 2H) 0.70-0.72 (m, 2H) 2.80-2.83 (m, 1H) 6.12 (s, 2H) 6.18 (s, 1H) 6.71 (s, 1H) 6.93 (s, 2H) 7.22-7.24 (d, J=6 Hz, 1H) 8.00 (s, 1H). MS ma/z calculated for (M+H)⁺ 377, found 377. Analytical HPLC retention time: 4.77 minutes

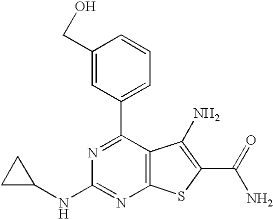

5-amino-2-(cyclopropylamino)-4-[3-(hydroxymethyl)phenyl]thiopheno[2,3-d]pyrimidine-6-carboxamide ¹H NMR (300 MHz, DMSO-d₆): δ ppm 0.50-0.54 (m, 2H) 0.70-0.72 (m, 2H) 2.80-2.83 (m, 1H) 4.58-4.60 (d, J=6 Hz, 2H) 5.30-5.34 (m, 1H) 5.99 (s, 2H) 7.00 (s, 2H) 7.43-7.52 (m, 4H) 7.96 (s, 1H). MS m/z calculated for (M+H)⁺ 356, found 356. Analytical HPLC retention time: 4.17 minutes

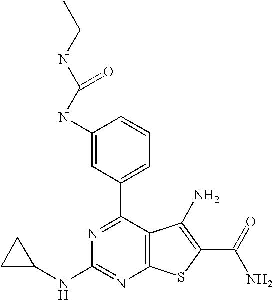

5-amino-2-(cyclopropylamino)-4-{3-[(ethylamino)carbonylamino]phenyl}thiopheno[2,3-d]pyrimidine-6-carboxamide ¹H NMR (300 MHz, DMSO-d₆): δ ppm 0.50-0.54 (m, 2H) 0.70-0.72 (m, 2H) 1.10-1.15 (m, 3H) 2.80-2.83 (m, 1H) 3.14-3.24 (m, 2H) 6.16 (s, 2H) 6.28 (s, 1H) 7.07 (s, 2H) 7.13-7.15 (d, J=6 Hz, 1H) 7.45-7.50 (m, 1H) 7.58-7.60 (d, J=6 Hz, 1H) 7.7 (s, 1H) 8.02 (s, 1H) 8.77 (s, 1H). MS m/z calculated for (M+H)⁺ 412, found 412. Analytical HPLC retention time: 4.37 minutes

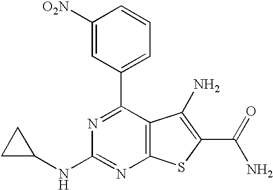

5-amino-2-(cyclopropylamino)-4-(3-nitrophenyl)thiopheno[2,3-d]pyrimidine-6-carboxamide ¹H NMR (300 MHz, DMSO-d₆): δ ppm 0.50-0.54 (m, 2H) 0.70-0.72 (m, 2H) 2.80-2.83 (m, 1H) 6.10 (s, 2H) 7.06 (s, 2H) 7.77-7.82 (m, 1H) 8.02-8.04 ((d, J=6 Hz, 2H) 8.38 (s, 1H) 8.4 (s, 1H). MS m/z calculated for (M+H)⁺ 371, found 371. Analytical HPLC retention time: 5.04 minutes

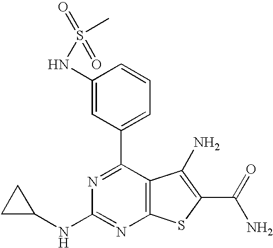

5-amino-2-(cyclopropylamino)-4-{3-[(methylsulfonyl)amino]phenyl}thiopheno[2,3-d]pyrimidine-6-carboxamide ¹H NMR (300 MHz, DMSO-d₆): δ ppm 0.50-0.54 (m, 2H) 0.70-0.72 (m, 2H) 2.80-2.83 (m, 1H) 3.05 (s, 3H) 6.10 (s, 2H) 7.06 (s, 2H) 7.27-7.38 (m, 3H) 7.39-7.54 (m, 1H) 8.00 (s, 1H) 10.00 (s, 1H). MS m/z calculated for (M+H)⁺ 419, found 419. Analytical HPLC retention time: 4.04 minutes

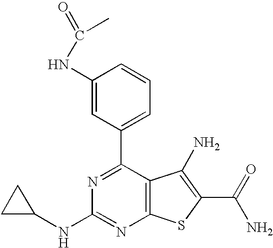

4-[3-(acetylamino)phenyl]-5-amino-2-(cyclopropylamino)thiopheno[2,3-d]pyrimidine-6-carboxamide ¹H NMR (300 MHz, DMSO-d₆): δ ppm 0.50-0.54 (m, 2H) 0.70-0.72 (m, 2H) 2.14 (s, 3H) 2.80-2.83 (m, 1H) 6.10 (s, 2H) 7.06 (s, 2H) 7.28-7.30 (d, J=6 Hz, 1H) 7.52-7.58 (t, 1H) 7.79-7.81 (d, J=6 Hz, 1H) 7.89 (s, 1H) 8.00 (s, 1H) 10.26 (s, 1H). MS m/z calculated for (M+H)⁺ 383, found 383. Analytical HPLC retention time: 4.24 minutes

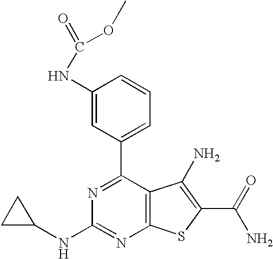

5-amino-2-(cyclopropylamino)-4-[3-(methoxycarbonylamino)phenyl]thiopheno[2,3-d]pyrimidine-6-carboxamide ¹H NMR (300 MHz, DMSO-d₆): δ ppm 0.50-0.54 (m, 2H) 0.70-0.72 (m, 2H) 2.80-2.83 (m, 1H) 3.68 (s, 3H) 6.02 (s, 2H) 7.00 (s, 2H) 7.16-7.18 (d, J=6 Hz, 1H) 7.47-7.49 (t, 1H) 7.63-7.65 (d, J=6 Hz, 2H) 7.97 (s, 1H) 9.91 (s, 1H). MS m/z calculated for (M+H)⁺ 399, found 399. Analytical HPLC retention time: 4.62 minutes

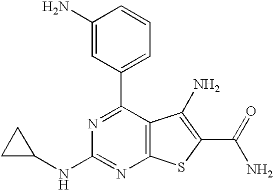

5-amino-4-(3-aminophenyl)-2-(cyclopropylamino)thieno[2,3-d]pyrimidine-6-carboxamide ¹H NMR (300 MHz, DMSO-d6): δ ppm 0.50-0.54 (m, 2H) 0.70-0.72 (m, 2H) 2.80-2.83 (m, 1H) 5.40 (s, 2H) 6.10 (s, 2H) 6.60-6.62 (d, J=6 Hz, 1H) 6.67 (s, 1H) 6.70-6.73 (d, J=9 Hz, 1H) 6.96 (s, 2H) 7.15-7.20 (t, 1H) 7.91 (s, 1H). MS m/z calculated for (M+H)⁺ 341, found 341. Analytical HPLC retention time: 3.74 minutes

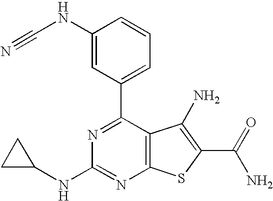

5-amino-4-[3-(cyanoamino)phenyl]-2-(cyclopropylamino)thiopheno[2,3-d]pyrimidine-6-carboxamide ¹H NMR (400 MHz, DMSO-d6): δ ppm 0.50-0.54 (m, 2H) 0.70-0.72 (m, 2H) 2.79-2.83 (m, 1H) 6.10 (s, 2H) 6.75-6.77 (d, J=8 Hz, 1H) 6.86-6.89 (m, 2H) 6.89 (s, 1H) 9.94 (s, 2H) 7.24-7.28 (t, 1H), 7.91 (s, 1H). MS m/z calculated for (M+H)⁺ 366, found 366. Analytical HPLC retention time: 4.46 minutes

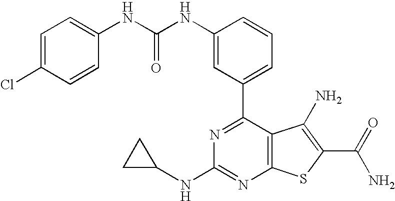

5-amino-4-(3-{[(4-chlorophenyl)amino]carbonylamino}phenyl)-2-(cyclopropylamino)thiopheno[2,3-d]pyrimidine-6-carboxamide ¹H NMR (400 MHz, DMSO-d6): δ ppm 0.51-0.54 (m, 2H) 0.70-0.72 (m, 2H) 2.80-2.84 (m, 1H) 6.10 (s, 2H) 7.00 (s, 2H) 7.16-7.18 (d, J=8 Hz, 1H) 7.31-7.35 (m, 2H) 7.45-7.49 (m, 3H) 7.58-7.60 (d, J=8 Hz, 1H) 7.70 (s, 1H) 7.97 (s, 1H) 8.90 (s, 1H) 8.96 (s, 1H). MS m/z calculated for (M+H)⁺ 494, found 494. Analytical HPLC retention time: 5.66 minutes

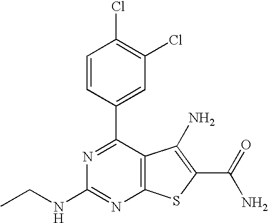

5-Amino-4-(3,4-dichloro-phenyl)-2-ethylamino-thieno[2,3-d]pyrimidine-6-carboxylic acid amide ¹H NMR (400 MHz, D6-DMSO) δ ppm 7.85 (d, 1H, J=2), 7.79 (d, 1H, J=8), 7.58 (m, 1H), 7.00 (s, 2H), 6.14 (bs, 2H), 3.37 (m, 2H), 1.55 (t, 3H, J=7). MS m/z calculated for (M+H)⁺ 382, found 382.

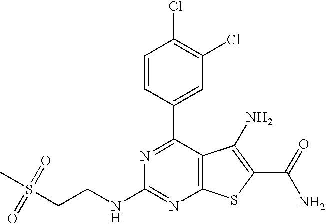

5-Amino-4-(3,4-dichloro-phenyl)-2-(2-methanesulfonyl-ethylamino)-thieno[2,3-d]pyrimidine-6-carboxylic acid amide ¹H NMR (400 MHz, D6-DMSO) δ ppm 7.90 (m, 2H), 7.79 (d, 1H, J=8), 7.59 (m, 1H), 7.06 (s, 2H), 6.19 (bs, 2H), 3.76 (m, 2H), 3.41 (t, 2H, J=7), 3.01 (s, 3H). MS m/z calculated for (M+H)⁺ 460, found 460.

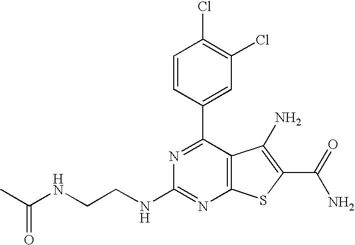

2-(2-Acetylamino-ethylamino)-5-amino-4-(3,4-dichloro-phenyl)-thieno[2,3-d]pyrimidine-6-carboxylic acid amide ¹H NMR (400 MHz, D6-DMSO) δ ppm 7.95 (bs, 1H), 7.85 (m, 1H), 7.79 (d, 1H, J=8), 7.58 (m, 1H), 7.02 (s, 2H), 6.15 (bs, 2H), 3.39 (m, 2H), 3.23 (m, 2H), 1.79 (s, 3H). MS m/z calculated for (M+H)⁺ 439, found 439.

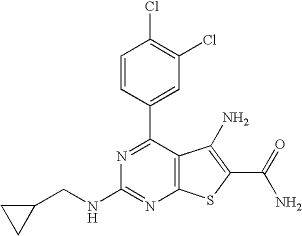

5-Amino-2-(cyclopropylmethyl-amino)-4-(3,4-dichloro-phenyl)-thieno[2,3-d]pyrimidine-6-carboxylic acid amide ¹H NMR (400 MHz, D6-DMSO) δ ppm 7.86 (d, 2H, J=2), 7.79 (d, 1H, J=8), 7.58 (m, 1H), 7.01 (s, 2H), 6.14 (bs, 2H), 3.21 (t, 2H, J=6), 1.08 (m, 1H), 0.42 (m, 2H), 0.23 (m, 2H).). MS m/z calculated for (M+H)⁺ 408, found 408.

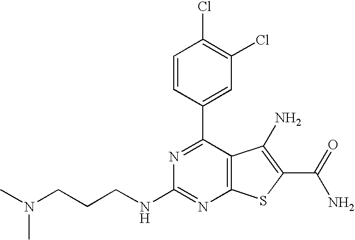

5-Amino-4-(3,4-dichloro-phenyl)-2-(3-dimethylamino-propylamino)-thieno[2,3-d]pyrimidine-6-carboxylic acid amide. ¹H NMR (400 MHz, D6-DMSO) δ ppm 7.85 (bs, 1H), 7.79 (d, 1H, J=8), 7.58 (m, 1H), 7.01 (s, 2H), 6.13 (bs, 2H), 3.34 (m, 2H), 2.26 (m, 2H), 2.12 (s, 6H), 1.66 (m, 2H). MS m/z calculated for (M+H)⁺ 439, found 439.

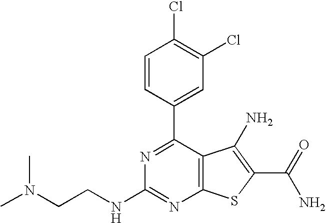

5-Amino-4-(3,4-dichloro-phenyl)-2-(2-dimethylamino-ethylamino)-thieno[2,3-d]pyrimidine-6-carboxylic acid amide ¹H NMR (400 MHz, D6-DMSO) δ ppm 7.86 (bs, 1H), 7.79 (d, 1H, J=8), 7.62 (m, 2H), 7.02 (s, 2H), 6.15 (bs, 2H), 3.41 (m, 2H), 2.42 (t, 2H, J=7), 2.07 (s, 6H). MS m/z calculated for (M+H)⁺ 425, found 425.

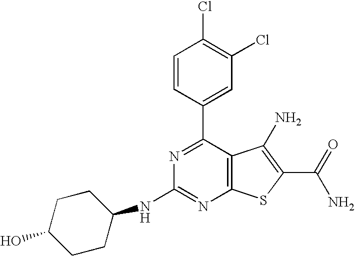

5-Amino-4-(3,4-dichloro-phenyl)-2-(4-hydroxy-cyclohexylamino)-thieno[2,3-d]pyrimidine-6-carboxylic acid amide ¹H NMR (400 MHz, D6-DMSO) δ ppm 7.85 (d, 1H, J=2), 7.77 (d, 1H, J=7), 7.71 (m, 1H), 7.57 (m, 1H), 6.99 (s, 2H), 6.12 (bs, 2H), 3.71 (m, 1H), 3.39 (m, 1H), 1.86 (m, 4H), 1.31 (m, 4H).). MS m/z calculated for (M+H)⁺ 452, found 452.

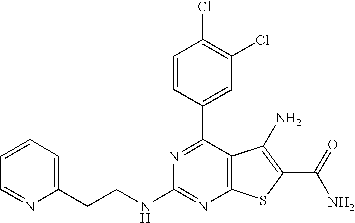

5-Amino-4-(3,4-dichloro-phenyl)-2-(2-pyridin-2-yl-ethylamino)-thieno[2,3-d]pyrimidine-6-carboxylic acid amide ¹H NMR (400 MHz, D6-DMSO) δ ppm 8.50 (m, 1H), 7.91 (m, 1H), 7.83 (d, 1H, J=8), 7.70 (m, 1H), 7.58 (m, 1H), 7.29 (m, 1H), 7.22 (m, 1H), 7.03 (s, 2H), 6.15 (bs, 2H), 3.70 (m, 2H), 3.03 (t, 2H, J=7). MS m/z calculated for (M+H)⁺ 459, found 459.

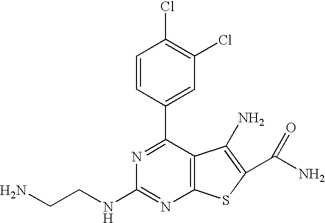

5-Amino-2-(2-amino-ethylamino)-4-(3,4-dichloro-phenyl)-thieno[2,3-d]pyrimidine-6-carboxylic acid amide ¹H NMR (400 MHz, D6-DMSO) δ ppm 7.85 (d, 1H, J=2), 7.79 (d, 1H, J=8), 7.57 (m, 1H), 7.00 (s, 2H), 6.14 (bs, 2H), 3.33 (m, 2H), 2.70 (m, 2H). MS m/z calculated for (M+H)⁺ 397, found 397.

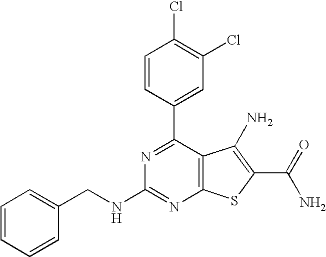

5-Amino-2-benzylamino-4-(3,4-dichloro-phenyl)-thieno[2,3-d]pyrimidine-6-carboxylic acid amide ¹H NMR (400 MHz, D6-DMSO) δ ppm 7.87 (bs, 1H), 7.78 (d, 1H, J=8), 7.57 (d, 1H, J=7), 7.30 (m, 4H), 7.23 (m, 1H), 7.03 (s, 2H), 6.15 (bs, 2H), 4.56 (d, 2H, J=6). MS m/z calculated for (M+H)⁺ 444, found 444.

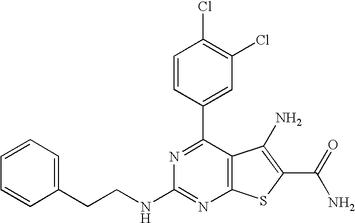

5-Amino-4-(3,4-dichloro-phenyl)-2-phenethylamino-thieno[2,3-d]pyrimidine-6-carboxylic acid amide. ¹H NMR (400 MHz, D6-DMSO) δ ppm 7.85 (m, 1H), 7.79 (d, 1H, J=8), 7.58 (m, 1H), 7.28 (m, 4H), 7.20 (m, 1H), 7.03 (s, 2H), 6.18 (bs, 2H), 3.56 (m, 2H), 2.50 (t, 2H, J=8). MS m/z calculated for (M+H)⁺ 458, found 458.

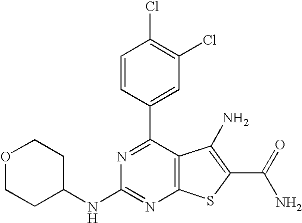

5-Amino-4-(3,4-dichloro-phenyl)-2-(tetrahydro-pyran-4-ylamino)-thieno[2,3-d]pyrimidine-6-carboxylic acid amide. ¹H NMR (400 MHz, D6-DMSO) δ ppm 7.86 (d, 1H, J=2), 7.79 (d, 1H, J=8), 7.58 (d, 1H, J=7), 7.01 (s, 2H), 6.14 (bs, 2H), 4.02 (m, 1H), 3.86 (m, 2H), 3.39 (m, 2H), 1.84 (m, 2H), 1.54 (m, 2H). MS 7/z calculated for (M+H)⁺ 438, found 438.

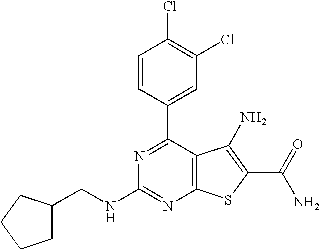

5-Amino-2-(cyclopentylmethyl-amino)-4-(3,4-dichloro-phenyl)-thieno[2,3-d]pyrimidine-6-carboxylic acid amide. ¹H NMR (400 MHz, D6-DMSO) δ ppm 7.85 (d, 1H, J=2), 7.79 (d, 1H, J=8), 7.57 (m, 1H), 7.01 (s, 2H), 6.13 (bs, 2H), 3.26 (m, 2H), 2.17 (m, 1H), 1.69 (m, 2H), 1.55 (m, 2H), 1.50 (m, 2H). MS m/z calculated for (M+H)⁺ 436, found 436.

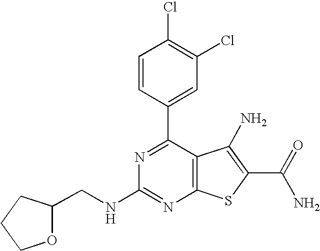

5-Amino-4-(3,4-dichloro-phenyl)-2-[(tetrahydro-furan-2-ylmethyl)-amino]-thieno[2,3-d]pyrimidine-6-carboxylic acid amide. ¹H NMR (400 MHz, D6-DMSO) δ ppm 7.86 (bs, 1H), 7.79 (d, 1H, J=8), 7.59 (m, 1H), 7.03 (s, 2H), 6.15 (bs, 2H), 4.02 (m, 2H), 3.76 (m, 1H), 3.61 (m, 1H), 3.45 (m, 1H) 1.82 (m, 3H), 1.58 (m, 1H). MS m/z calculated for (M+H)⁺ 438, found 438.

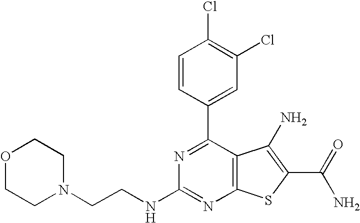

5-Amino-4-(3,4-dichloro-phenyl)-2-(2-morpholin-4-yl-ethylamino)-thieno[2,3-d]pyrimidine-6-carboxylic acid amide. ¹H NMR (400 MHz, D6-DMSO) δ ppm 7.86 (bs, 1H), 7.79 (d, 1H, J=8), 7.67 (m, 1H), 7.59 (m, 1H), 7.01 (s, 2H), 6.15 (bs, 2H), 3.57 (m, 4H), 3.47 (m, 2H), 2.53 (m, 2H), 2.46 (m, 4H). MS m/z calculated for (M+H)⁺ 467, found 467.

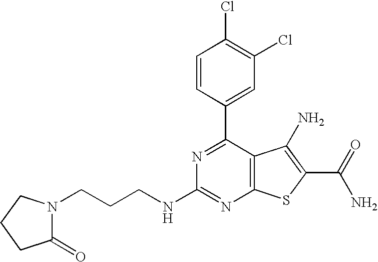

5-Amino-4-(3,4-dichloro-phenyl)-2-[3-(2-oxo-pyrrolidin-1-yl)-propylamino]-thieno[2,3-d]pyrimidine-6-carboxylic acid amide. ¹H NMR (400 MHz, D6-DMSO) δ ppm 7.86 (bs, 1H), 7.79 (d, 1H, J=8), 7.59 (m, 1H), 7.01 (s, 2H), 6.14 (bs, 2H), 3.27 (m, 6H), 2.20 (m, 2H), 1.92 (m, 2H), 1.73 (m, 2H). MS m/z calculated for (M+H)⁺ 479, found 479.

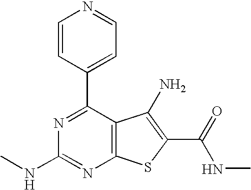

5-Amino-4-(4-pyridyl)-2-methylamino-thieno[2,3-d]pyrimidine-6-carboxylic acid methylamide. (DMSO-d6) δ 8.76 (bs. 2H), 7.77 (bs, 1H), 7.57 (bs, 2H), 7.49 (d, 1H), 5.95 (bs, 2H), 2.87 (d, 3H), 2.68 (d, 3H); MH+ 315.

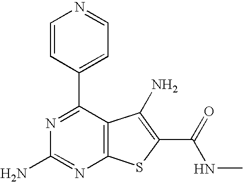

5-Amino-4-(4-pyridyl)-2-amino-thieno[2,3-d]pyrimidine-6-carboxylic acid methylamide. (DMSO-d6) δ 8.75 (d, 2H), 7.57 (d, 2H), 7.51 (m, 1H), 7.28 (bs, 1H), 5.96 (bs, 2H), 2.67 (d, 3H); MH+ 301; m.p.>250.

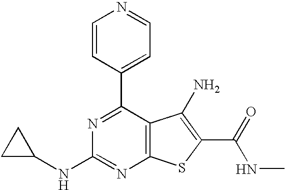

5-Amino-4-(4-pyridyl)-2-cyclopropylamino-thieno[2,3-d]pyrimidine-6-carboxylic acid methylamide. (DMSO-d6) δ 8.75 (d, 2H), 8.03 (bs, 1H), 7.57 (d, 2H), 7.50 (m, 1H), 5.96 (bs, 2H), 2.81 (m, 1H), 2.68 (d, 3H), 0.71 (m, 2H), 0.52 (m, 2H); MH+ 341; m.p.>250.

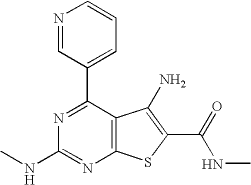

5-Amino-4-(3-pyridyl)-2-methylamino-thieno[2,3-d]pyrimidine-6-carboxylic acid methylamide. (DMSO) δ 8.76 (bs, 1H), 8.73 (d, 1H), 7.99 (m, 1H), 7.75 (bs, 1H), 7.57 (m, 1H), 7.5 (m, 1H), 5.97 (bs, 2H), 2.88 (d, 3H), 2.68 (d, 3H); MH+ 315; m.p.>240.

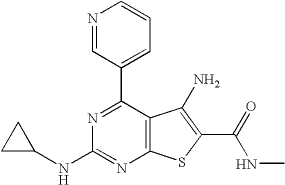

5-Amino-4-(3-pyridyl)-2-cyclopropylamino-thieno[2,3-d]pyrimidine-6-carboxylic acid methylamide. (DMSO) δ 8.78 (bs, if H), 8.73 (d, 1H), 7.99 (m, 2H), 7.57 (m, 1H), 7.52 (m, 1H), 5.98 (bs, 2H), 2.82 (m, 1H), 0.71 (m, 2H), 0.53 (m, 2H); MH+ 341; m.p. 225-226.

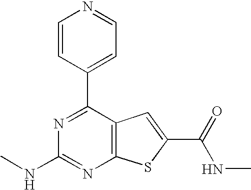

N-methyl[2-(methylamino)-4-(4-pyridyl)thiopheno[2,3-d]pyrimidin-6-yl}carboxamide. (DMSO) δ 8.84 (m, 1H), 8.69 (m, 1H), 8.03 (m, 1H), 7.83 (m, 2H), 2.92 (m, 3H), 2.78 (m, 3H); MH+ 300.

### EQUIVALENTS

The present disclosure is not to be limited in scope by the specific embodiments described herein. Indeed, various modifications of the embodiments provided herein in addition to those described will become apparent to those skilled in the art from the foregoing description. Such modifications are intended to fall within the scope of the appended claims.

Various publications are cited herein, the disclosures of which are incorporated by reference in their entireties.

What is claimed is:

1. A compound of the formula: 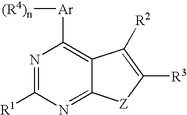
or a pharmaceutically acceptable salt thereof, wherein:
Ar is pyridine, napthyl or dihydrobenzofuran;
R¹ is C(O)NR⁵R⁶, NR⁵R⁶, O—C₁₋₈alkyl, CN, or substituted or unsubstituted C₁₋₈alkyl;
R² is H, NH₂, substituted or unsubstituted C₁₋₈alkyl or substituted or unsubstituted aryl;
R³ is C₁₋₈hydroxyalkyl or C(O)NR⁵R⁶;
R⁴ is at each occurrence independently halo, OH, NH₂, CN, NHCN, NO₂, substituted or unsubstituted C₁₋₈alkyl, substituted or unsubstituted O—C₁₋₈alkyl, substituted or unsubstituted O—C₁₋₈alkylene-aryl, substituted or unsubstituted aryl, substituted or unsubstituted O-aryl, substituted or unsubstituted heterocyclyl, NHS(O)₂—C₁₋₈alkyl, NHC(O)—C₁₋₈alkyl, NHC(O)O—C₁₋₈alkyl, NHC(O)NHC₁₋₈alkyl, NHC(O)NHC₀₋₈alkylene-substituted or unsubstituted phenyl, NR⁵R⁶, NHC(O)NHR⁵, NHC(O)R⁵, C(O)OC₁₋₈alkyl, or C(O)NR⁵R⁶; or two adjacent R₄ groups taken together represent —OCH₂O—, —OCH₂CH₂O—, or —NH [symbol] N—; or two adjacent R₄ groups taken together with Ar form naphthalene;
R⁵ and R⁶ are at each occurrence independently H, substituted or unsubstituted C₁₋₈alkyl, substituted or unsubstituted aryl, substituted or unsubstituted heterocyclyl, substituted or unsubstituted C₁₋₈alkylene-O—C₁₋₈alkyl, C₁₋₈alkylene-C(O)NH₂, C₁₋₈alkylene-C(O)OH, substituted or unsubstituted C₃₋₈cycloalkyl; or R⁵ and R⁶ together with the nitrogen atom to which they are attached form a 3-7 membered heterocyclyl;
Z is S, NH or O; and
n is an integer ranging from 0 to 5;
wherein groups that are substituted are substituted with one or more halogen; C₁₋₈ alkyl; C₂₋₈ alkenyl; C₂₋₈ alkynyl; hydroxyl; C₁₋₈ alkoxyl; amino; nitro; thiol; thioether; imine; cyano; amido; phosphonato; phosphine; carboxyl; thiocarbonyl; sulfonyl; sulfonamide; ketone; aldehyde; ester; carbonyl; haloalkyl; B(OH)₂; carbocyclic cycloalkyl, heterocycloalkyl, monocyclic or fused or non-fused polycyclic aryl or heteroaryl; amino; O-lower alkyl; O-aryl, aryl; aryl-lower alkyl; CO₂CH₃; CONH₂; OCH₂CONH₂; NH₂; SO₂NH₂; OCHF₂; CF₃; or OCF₃ groups, wherein each of these groups is optionally substituted.

2. A compound of claim 1, wherein R¹ is NR⁵R⁶.

3. A compound of claim 1, wherein R² is NH₂.

4. A compound of claim 1, wherein R³ is C(O)NH₂.

5. A compound of the formula: 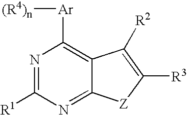
or a pharmaceutically acceptable salt thereof, wherein:
Ar is phenyl, napthyl or dihydrobenzofuran;
R¹ is C(O)NR⁵R⁶, NR⁵R⁶, O—C₁₋₈alkyl, CN, or substituted or unsubstituted C₁₋₈alkyl;
R² is H, NH₂, substituted or unsubstituted C₁₋₈alkyl or substituted or unsubstituted aryl;
R³ is C₁₋₈hydroxyalkyl or C(O)NR⁵R⁶;
R⁴ is at each occurrence independently halo, OH, NH₂, CN, NHCN, NO₂, substituted or unsubstituted C₁₋₈alkyl, substituted or unsubstituted O—C₁₋₈alkylene-aryl, substituted or unsubstituted aryl, substituted or unsubstituted O-aryl, substituted or unsubstituted heterocyclyl, NHS(O)₂—C₁₋₈alkyl, NHC(O)—C₁₋₈alkyl, NHC(O)O—C₁₋₈alkyl, NHC(O)NHC₁₋₈alkyl, NHC(O)NHC₀₋₈alkylene-substituted or unsubstituted phenyl, NR⁵R⁶, NHC(O)NHR⁵, NHC(O)R⁵, C(O)OC₁₋₈alkyl, or C(O)NR⁵R⁶; or two adjacent R₄ groups taken together represent —OCH₂O—, —OCH₂CH₂O—, or —NH [symbol] N—; or two adjacent R₄ groups taken together with Ar form naphthalene;
R⁵ and R⁶ are at each occurrence independently substituted or unsubstituted C₁₋₈alkyl, substituted or unsubstituted aryl, substituted or unsubstituted heterocyclyl, substituted or unsubstituted C₁₋₈alkylene-O—C₁₋₈alkyl, C₁₋₈alkylene-C(O)NH₂, C₁₋₈alkylene-C(O)OH, substituted or unsubstituted C₃₋₈cycloalkyl; or R⁵ and R⁶ together with the nitrogen atom to which they are attached form a 3-7 membered heterocyclyl;
Z is S, NH or O; and
n is an integer ranging from 1 to 5;
wherein groups that are substituted are substituted with one or more halogen; C₁₋₈ alkyl; C₂₋₈ alkenyl; C₂₋₈ alkynyl; hydroxyl; C₁₋₈ alkoxyl; amino; nitro; thiol; thioether; imine; cyano; amido; phosphonato; phosphine; carboxyl; thiocarbonyl; sulfonyl; sulfonamide; ketone; aldehyde; ester; carbonyl; haloalkyl; B(OH)₂; carbocyclic cycloalkyl, heterocycloalkyl, monocyclic or fused or non-fused polycyclic aryl or heteroaryl; amino; O-lower alkyl; O-aryl, aryl; aryl-lower alkyl; CO₂CH₃; CONH₂; OCH₂CONH₂; NH₂; SO₂NH₂; OCHF₂; CF₃; or OCF₃ groups, wherein each of these groups is optionally substituted.

6. A compound of the formula: 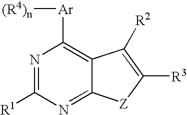
or a pharmaceutically acceptable salt thereof, wherein:
Ar is phenyl, napthyl or dihydrobenzofuran;
R¹ is C(O)NR⁵R⁶, NR⁵R⁶, O—C₁₋₈alkyl, CN, or substituted or unsubstituted C₁₋₈alkyl;
R² is NH₂;
R³ is C₁₋₈hydroxyalkyl or C(O)NR⁵R⁶;
R⁴ is at each occurrence independently halo, OH, NH₂, CN, NHCN, NO₂, substituted or unsubstituted C₁₋₈alkyl, substituted or unsubstituted aryl, substituted or unsubstituted O-aryl, substituted or unsubstituted heterocyclyl, NHS(O)₂—C₁₋₈alkyl, NHC(O)—C₁₋₈alkyl, NHC(O)O—C₁₋₈alkyl, NHC(O)NHC₁₋₈alkyl, NHC(O)NHC₀₋₈alkylene-substituted or unsubstituted phenyl, NR⁵R⁶, NHC(O)NHR⁵, NHC(O)R⁵, C(O)OC₁₋₈alkyl, or C(O)NR⁵R⁶; or two adjacent R₄ groups taken together represent —OCH₂O—, —OCH₂CH₂O—, or —NH [symbol] N—; or two adjacent R₄ groups taken together with Ar form naphthalene;
R⁵ and R⁶ are at each occurrence independently H, substituted or unsubstituted C₁₋₈alkyl, substituted or unsubstituted aryl, substituted or unsubstituted heterocyclyl, substituted or unsubstituted C₁₋₈alkylene-O—C₁₋₈alkyl, C₁₋₈alkylene-C(O)NH₂, C₁₋₈alkylene-C(O)OH, substituted or unsubstituted C₃₋₈cycloalkyl; or R⁵ and R⁶ together with the nitrogen atom to which they are attached form a 3-7 membered heterocyclyl;
Z is S, NH or O; and
n is an integer ranging from 1 to 5;
wherein groups that are substituted are substituted with one or more halogen; C₁₋₈ alkyl; C₂₋₈ alkenyl; C₂₋₈ alkynyl; hydroxyl; C₁₋₈ alkoxyl; amino; nitro; thiol; thioether; imine; cyano; amido; phosphonato; phosphine; carboxyl; thiocarbonyl; sulfonyl; sulfonamide; ketone; aldehyde; ester; carbonyl; haloalkyl; B(OH)₂; carbocyclic cycloalkyl, heterocycloalkyl, monocyclic or fused or non-fused polycyclic aryl or heteroaryl; amino; O-lower alkyl; O-aryl, aryl; aryl-lower alkyl; CO₂CH₃; CONH₂; OCH₂CONH₂; NH₂; SO₂NH₂; OCHF₂; CF₃; or OCF₃ groups, wherein each of these groups is optionally substituted.

7. A compound of the formula: 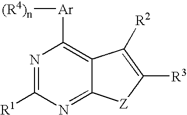
or a pharmaceutically acceptable salt thereof, wherein:
Ar is phenyl, napthyl or dihydrobenzofuran;
R¹ is C(O)NR⁵R⁶, NR⁵R⁶, O—C₁₋₈alkyl, CN, or substituted or unsubstituted C₁₋₈alkyl;
R² is H, NH₂, substituted or unsubstituted C₁₋₈alkyl or substituted or unsubstituted aryl;
R³ is C₁₋₈hydroxyalkyl or C(O)(3-7 membered heterocyclyl);
R⁴ is at each occurrence independently halo, OH, NH₂, CN, NHCN, NO₂, substituted or unsubstituted C₁₋₈alkyl, substituted or unsubstituted O—C₁₋₈alkylene-aryl, substituted or unsubstituted O—C₁₋₈alkyl, substituted or unsubstituted aryl, substituted or unsubstituted O-aryl, substituted or unsubstituted heterocyclyl, NHS(O)₂—C₁₋₈alkyl, NHC(O)—C₁₋₈alkyl, NHC(O)O—C₁₋₈alkyl, NHC(O)NHC₁₋₈alkyl, NHC(O)NHC₀₋₈alkylene-substituted or unsubstituted phenyl, NR⁵R⁶, NHC(O)NHR⁵, NHC(O)R⁵, C(O)OC₁₋₈alkyl, or C(O)NR⁵R⁶; or two adjacent R₄ groups taken together represent —OCH₂O—, —OCH₂CH₂O—, or —NH [symbol] N—; or two adjacent R₄ groups taken together with Ar form naphthalene;
R⁵ and R⁶ are at each occurrence independently H, substituted or unsubstituted C₁₋₈alkyl, substituted or unsubstituted aryl, substituted or unsubstituted heterocyclyl, substituted or unsubstituted C₁₋₈alkylene-O—C₁₋₈alkyl, C₁₋₈alkylene-C(O)NH₂, C₁₋₈alkylene-C(O)OH, substituted or unsubstituted C₃₋₈cycloalkyl; or R⁵ and R⁶ together with the nitrogen atom to which they are attached form a 3-7 membered heterocyclyl;
Z is S, NH or O; and
n is an integer ranging from 0 to 5;
wherein groups that are substituted are substituted with one or more halogen; C₁₋₈ alkyl; C₂₋₈ alkenyl; C₂₋₈ alkynyl; hydroxyl; C₁₋₈ alkoxyl; amino; nitro; thiol; thioether; imine; cyano; amido; phosphonato; phosphine; carboxyl; thiocarbonyl; sulfonyl; sulfonamide; ketone; aldehyde; ester; carbonyl; haloalkyl; B(OH)₂; carbocyclic cycloalkyl, heterocycloalkyl, monocyclic or fused or non-fused polycyclic aryl or heteroaryl; amino; O-lower alkyl; O-aryl, aryl; aryl-lower alkyl; CO₂CH₃; CONH₂; OCH₂CONH₂; NH₂; SO₂NH₂; OCHF₂; CF₃; or OCF₃ groups, wherein each of these groups is optionally substituted.

8. A compound of the formula: 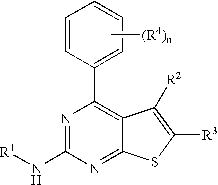
or a pharmaceutically acceptable salt thereof, wherein:
R¹ is substituted or unsubstituted C₁₋₈alkyl, substituted or unsubstituted C₁₋₈alkylene-O—C₁₋₈alkyl or substituted or unsubstituted C₃₋₈cycloalkyl;
R² is H, NH₂, substituted or unsubstituted C₁₋₈alkyl or substituted or unsubstituted aryl;
R³ is C₁₋₈hydroxyalkyl or C(O)NR⁵R⁶;
R⁴ is at each occurrence independently halo, substituted or unsubstituted C₁₋₈alkyl, substituted or unsubstituted O—C₁₋₈alkyl, substituted or unsubstituted O—C₁₋₈alkylene-aryl, substituted or unsubstituted aryl, substituted or unsubstituted O-aryl, substituted or unsubstituted heterocyclyl or C(O)NR⁵R⁶; or two adjacent R₄ groups taken together represent —OCH₂O— or —OCH₂CH₂O—;
R⁵ and R⁶ are at each occurrence independently H, substituted or unsubstituted C₁₋₈alkyl, C₁₋₈alkylene-C(O)NH₂, C₁₋₈alkylene-C(O)OH, substituted or unsubstituted aryl or substituted or unsubstituted heterocyclyl; or R⁵ and R⁶ together with the nitrogen atom to which they are attached form a 3-7 membered heterocyclyl; and
n is an integer ranging from 0 to 5;
wherein groups that are substituted are substituted with one or more halogen; C₁₋₈ alkyl; C₂₋₈ alkenyl; C₂₋₈ alkynyl; hydroxyl; C₁₋₈ alkoxyl; amino; nitro; thiol; thioether; imine; cyano; amido; phosphonato; phosphine; carboxyl; thiocarbonyl; sulfonyl; sulfonamide; ketone; aldehyde; ester; carbonyl; haloalkyl; B(OH)₂; carbocyclic cycloalkyl, heterocycloalkyl, monocyclic or fused or non-fused polycyclic aryl or heteroaryl; amino; O-lower alkyl; O-aryl, aryl; aryl-lower alkyl; CO₂CH₃; CONH₂; OCH₂CONH₂; NH₂; SO₂NH₂; OCHF₂; CF₃; or OCF₃ groups, wherein each of these groups is optionally substituted.

9. A compound of claim 8, wherein R² is NH₂.

10. A compound of claim 8, wherein R⁵ and R⁶ together with the nitrogen atom to which they are attached form a benzofuranyl, benzothienyl, indolyl, benzopyrazolyl, coumarinyl, isoquinolinyl, pyrrolyl, thienyl, furanyl, thiazolyl, imidazolyl, pyrazolyl, triazolyl, quinolinyl, pyrimidinyl, pyridinyl, piperizinyl, pyridonyl, pyrazinyl, pyridazinyl, isothiazolyl, isoxazolyl, (1,4)-dioxane, (1,3)-dioxolane, 4,5-dihydro-1H-imidazolyl or tetrazolyl ring.

11. A compound of the formula: 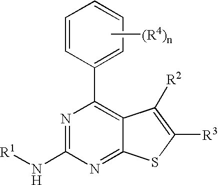
or a pharmaceutically acceptable salt thereof, wherein:
R¹ is H, substituted or unsubstituted C₁₋₈alkyl, substituted or unsubstituted C₁₋₈alkylene-O—C₁₋₈alkyl or substituted or unsubstituted C₃₋₈cycloalkyl;
R² is NH₂;
R³ is C₁₋₈hydroxyalkyl or C(O)NR⁵R⁶;
R⁴ is at each occurrence independently halo, substituted or unsubstituted C₁₋₈alkyl, substituted or unsubstituted O—C₁₋₈alkyl, substituted or unsubstituted O—C₁₋₈alkylene-aryl, substituted or unsubstituted aryl, substituted or unsubstituted O-aryl, substituted or unsubstituted heterocyclyl or C(O)NR⁵R⁶; or two adjacent R₄ groups taken together represent —OCH₂O— or —OCH₂CH₂O—;
R⁵ and R⁶ are at each occurrence independently H, substituted or unsubstituted C₁₋₈alkyl, C₁₋₈alkylene-C(O)NH₂, C₁₋₈alkylene-C(O)OH, substituted or unsubstituted aryl or substituted or unsubstituted heterocyclyl; or R⁵ and R⁶ together with the nitrogen atom to which they are attached form a 3-7 membered heterocyclyl; and
n is an integer ranging from 0 to 5;
wherein groups that are substituted are substituted with one or more halogen; C₁₋₈ alkyl; C₂₋₈ alkenyl; C₂₋₈ alkynyl; hydroxyl; C₁₋₈ alkoxyl; amino; nitro; thiol; thioether; imine; cyano; amido; phosphonato; phosphine; carboxyl; thiocarbonyl; sulfonyl; sulfonamide; ketone; aldehyde; ester; carbonyl; haloalkyl; B(OH)₂; carbocyclic cycloalkyl, heterocycloalkyl, monocyclic or fused or non-fused polycyclic aryl or heteroaryl; amino; O-lower alkyl; O-aryl, aryl; aryl-lower alkyl; CO₂CH₃; CONH₂; OCH₂CONH₂; NH₂; SO₂NH₂; OCHF₂; CF₃; or OCF₃ groups, wherein each of these groups is optionally substituted.

12. A compound of the formula: 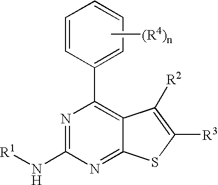
or a pharmaceutically acceptable salt thereof, wherein:
R¹ is H, substituted or unsubstituted C₁₋₈alkyl, substituted or unsubstituted C₁₋₈alkylene-O—C₁₋₈alkyl or substituted or unsubstituted C₃₋₈cycloalkyl;
R² is H, NH₂, substituted or unsubstituted C₁₋₈alkyl or substituted or unsubstituted aryl;
R³ is C₁₋₈hydroxyalkyl or C(O)NR⁵R⁶;
R⁴ is at each occurrence independently halo, substituted or unsubstituted C₁₋₈alkyl, substituted or unsubstituted O—C₁₋₈alkyl, substituted or unsubstituted O—C₁₋₈alkylene-aryl, substituted or unsubstituted aryl, substituted or unsubstituted O-aryl, substituted or unsubstituted heterocyclyl or C(O)NR⁵R⁶; or two adjacent R₄ groups taken together represent —OCH₂O— or —OCH₂CH₂O—;
R⁵ and R⁶ together with the nitrogen atom to which they are attached form a benzofuranyl, benzothienyl, indolyl, benzopyrazolyl, coumarinyl, isoquinolinyl, pyrrolyl, thienyl, furanyl, thiazolyl, imidazolyl, pyrazolyl, triazolyl, quinolinyl, pyrimidinyl, pyridinyl, piperizinyl, pyridonyl, pyrazinyl, pyridazinyl, isothiazolyl, isoxazolyl, (1,4)-dioxane, (1,3)-dioxolane, 4,5-dihydro-1H-imidazolyl or tetrazolyl ring; and
n is an integer ranging from 0 to 5;
wherein groups that are substituted are substituted with one or more halogen; C₁ g alkyl; C₂₋₈ alkenyl; C₂₋₈ alkynyl; hydroxyl; C₁₋₈ alkoxyl; amino; nitro; thiol; thioether; imine; cyano; amido; phosphonato; phosphine; carboxyl; thiocarbonyl; sulfonyl; sulfonamide; ketone; aldehyde; ester; carbonyl; haloalkyl; B(OH)₂; carbocyclic cycloalkyl, heterocycloalkyl, monocyclic or fused or non-fused polycyclic aryl or heteroaryl; amino; O-lower alkyl; O-aryl, aryl; aryl-lower alkyl; CO₂CH₃; CONH₂; OCH₂CONH₂; NH₂; SO₂NH₂; OCHF₂; CF₃; or OCF₃ groups, wherein each of these groups is optionally substituted.

13. A compound of claim 5, 11 or 12, wherein R³ is C(O)NR⁵R⁶.

14. A compound of the formula: 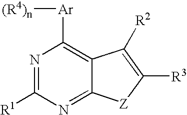
or a pharmaceutically acceptable salt thereof, wherein:
R¹ is H, substituted or unsubstituted C₁₋₈alkyl, substituted or unsubstituted C₁₋₈alkylene-O—C₁₋₈alkyl or substituted or unsubstituted C₃₋₈cycloalkyl;
R² is H, NH₂, substituted or unsubstituted C₁₋₈alkyl or substituted or unsubstituted aryl;
R³ is C₁₋₈hydroxyalkyl or C(O)NR⁵R⁶;
R⁴ is at each occurrence independently halo, substituted or unsubstituted C₁₋₈alkyl, substituted or unsubstituted O—C₁₋₈alkyl, substituted or unsubstituted O—C₁₋₈alkylene-aryl, substituted or unsubstituted aryl, substituted or unsubstituted O-aryl, substituted or unsubstituted heterocyclyl or C(O)NR⁵R⁶; or two adjacent R₄ groups taken together represent —OCH₂O— or —OCH₂CH₂O—;
R⁵ and R¹ are at each occurrence independently H, substituted or unsubstituted C₁₋₈alkyl, C₁₋₈alkylene-C(O)NH₂, C₁₋₈alkylene-C(O)OH, substituted or unsubstituted aryl or substituted or unsubstituted heterocyclyl; or R⁵ and R⁶ together with the nitrogen atom to which they are attached form a 3-7 membered heterocyclyl;
Z is NH or O; and
n is an integer ranging from 0 to 5;
wherein groups that are substituted are substituted with one or more halogen; C₁₋₈ alkyl; C₂₋₈ alkenyl; C₂₋₈ alkynyl; hydroxyl; C₁₋₈ alkoxyl; amino; nitro; thiol; thioether; imine; cyano; amido; phosphonato; phosphine; carboxyl; thiocarbonyl; sulfonyl; sulfonamide; ketone; aldehyde; ester; carbonyl; haloalkyl; B(OH)₂; carbocyclic cycloalkyl, heterocycloalkyl, monocyclic or fused or non-fused polycyclic aryl or heteroaryl; amino; O-lower alkyl; O-aryl, aryl; aryl-lower alkyl; CO₂CH₃; CONH₂; OCH₂CONH₂; NH₂; SO₂NH₂; OCHF₂; CF₃; or OCF₃ groups, wherein each of these groups is optionally substituted.

15. A compound of the formula: 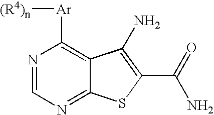
or a pharmaceutically acceptable salt thereof, wherein:
Ar is phenyl, pyridine or dihydrobenzofuran;
R⁴ is at each occurrence independently halo, OH, NO₂, substituted or unsubstituted O—C₁₋₈alkyl, substituted or unsubstituted O—C₁₋₈alkylene-aryl, NHC(O)NHC₀₋₈alkylene-substituted or unsubstituted phenyl, NR⁵R⁶, NHC(O)NHR⁵, or NHC(O)R⁵;
R⁵ and R⁶ are at each occurrence independently H, substituted or unsubstituted C₁₋₈alkyl, substituted or unsubstituted aryl, substituted or unsubstituted heterocyclyl, substituted or unsubstituted C₁₋₈alkylene-O—C₁₋₈alkyl, C₁₋₈alkylene-C(O)NH₂, C₁₋₈alkylene-C(O)OH, substituted or unsubstituted C₃₋₈cycloalkyl; or R⁵ and R⁶ together with the nitrogen atom to which they are attached form a 3-7 membered heterocyclyl; and
n is an integer ranging from 0 to 5.

16. A compound or a pharmaceutically acceptable salt thereof having the structure:

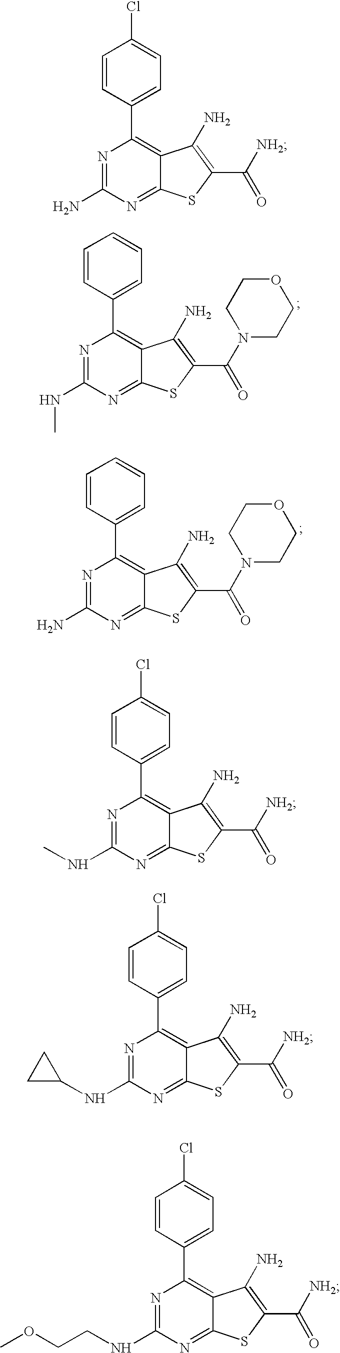

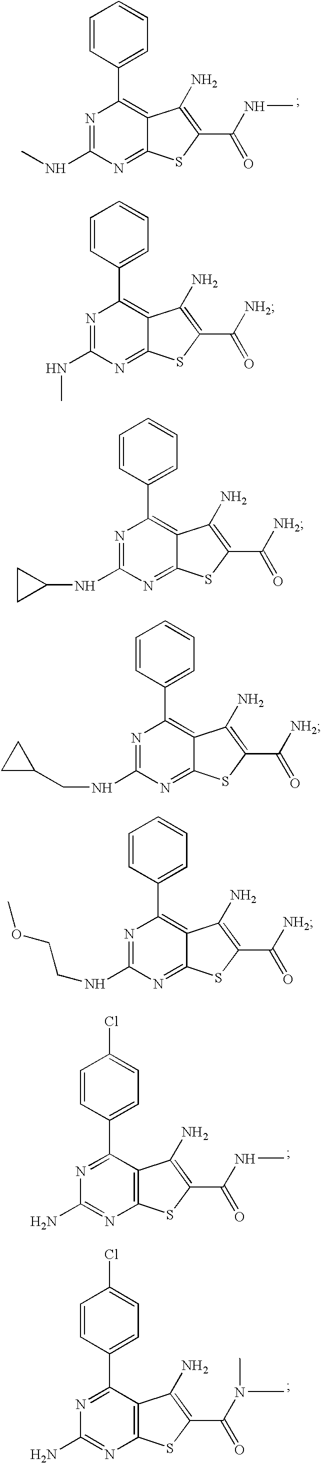

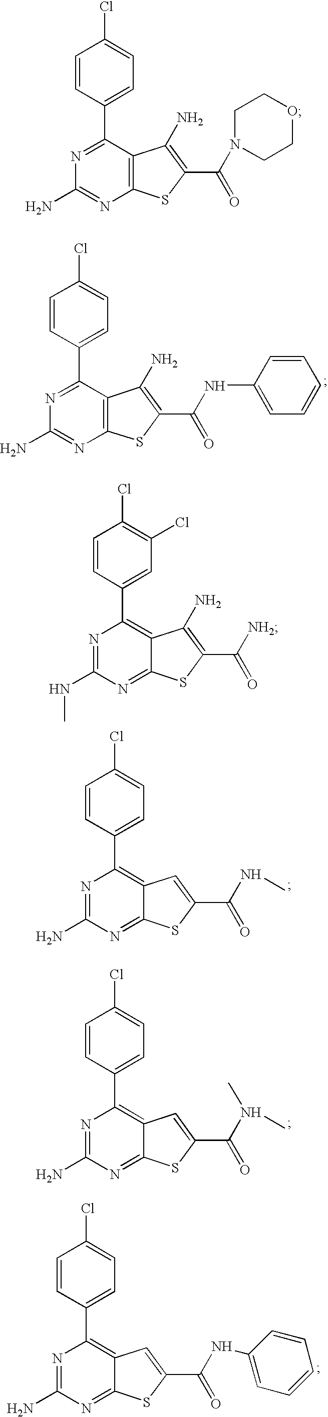

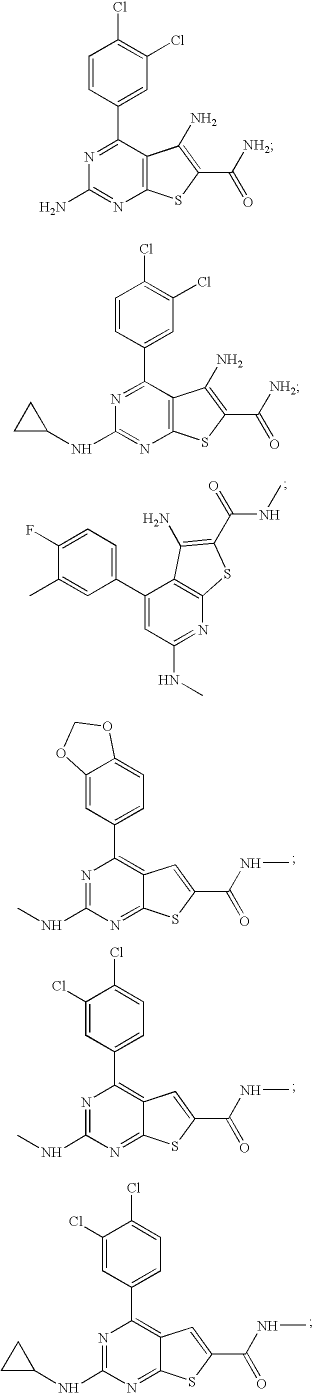

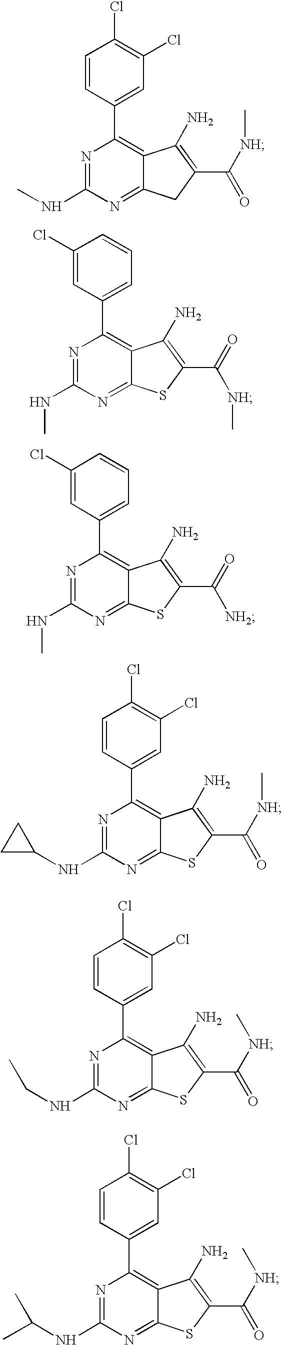

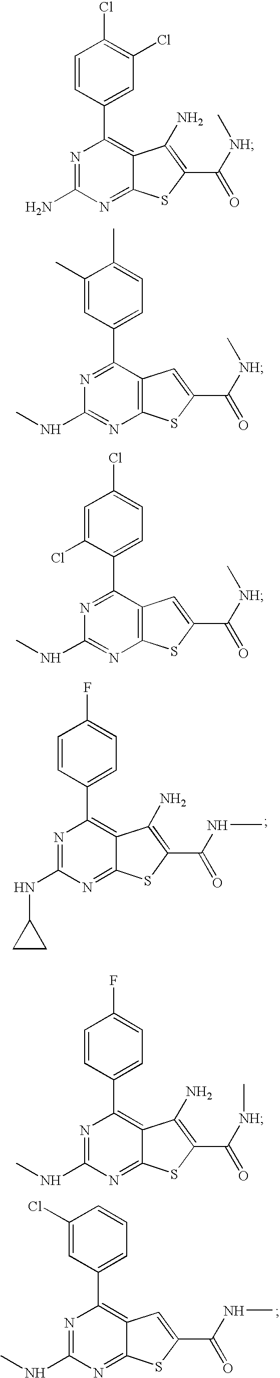

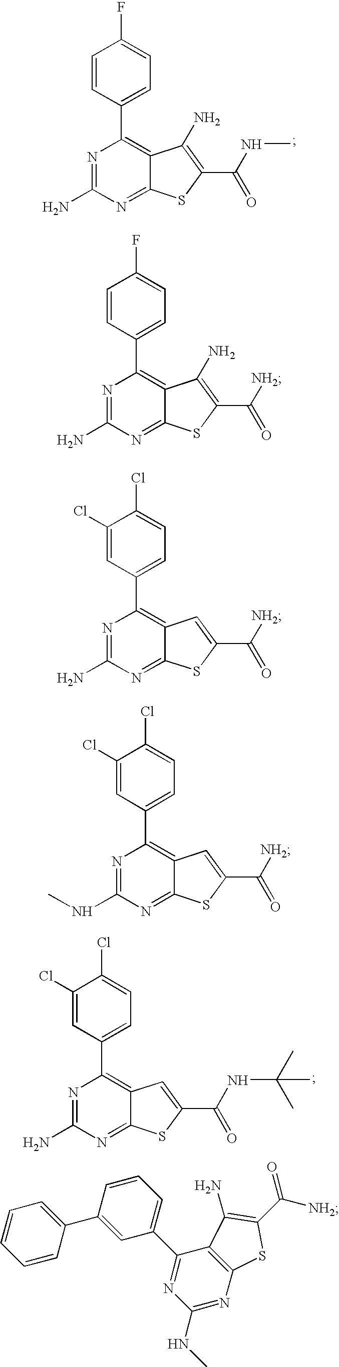

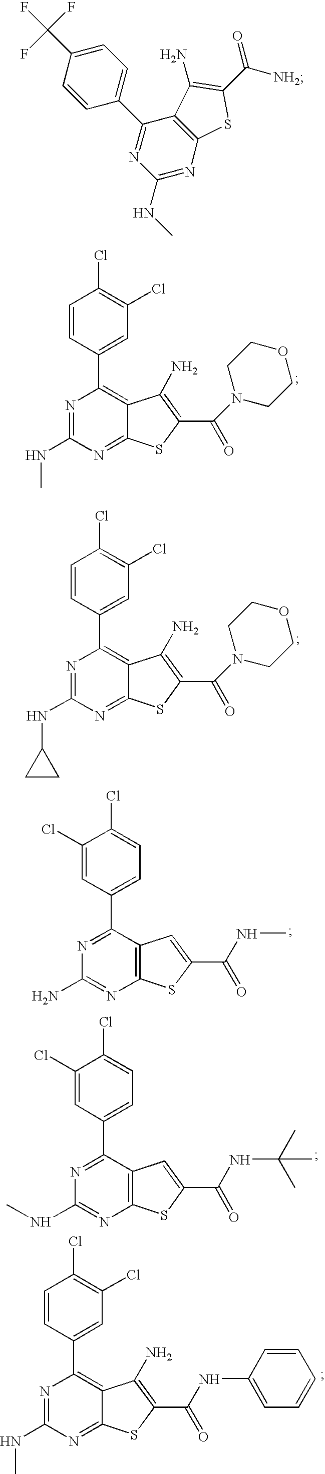

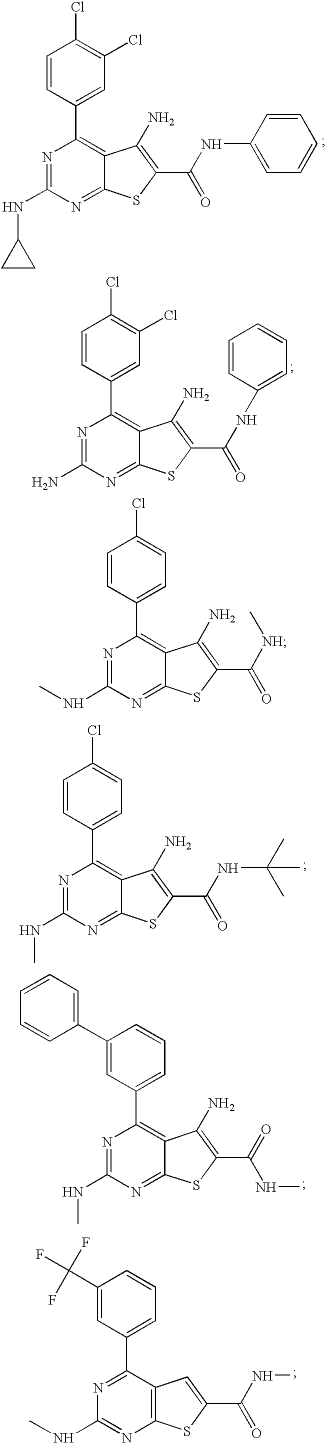

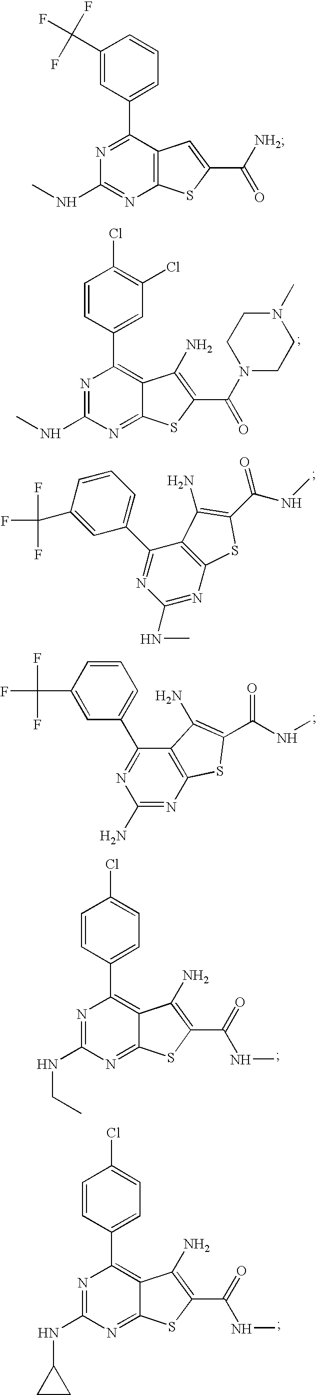

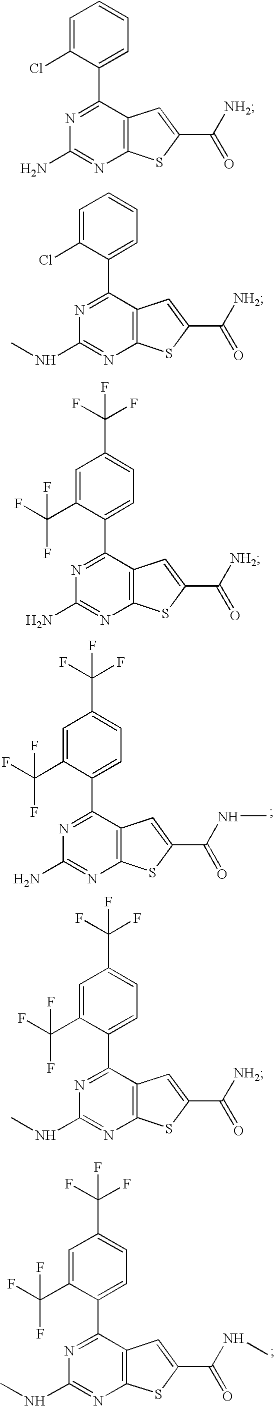

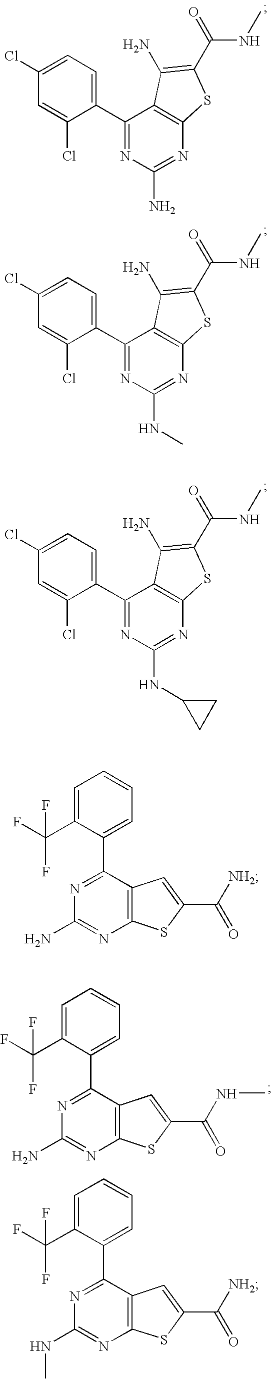

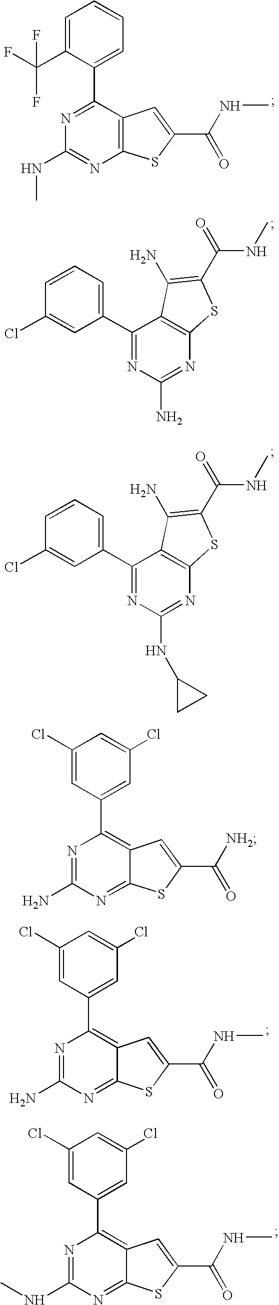

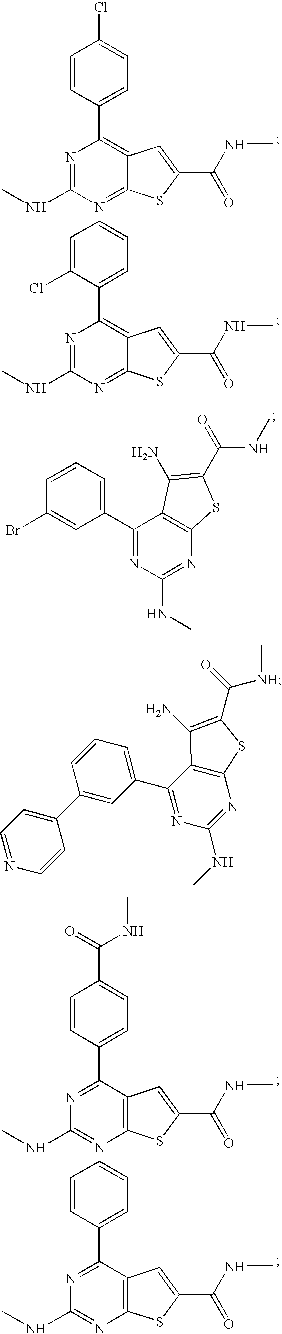

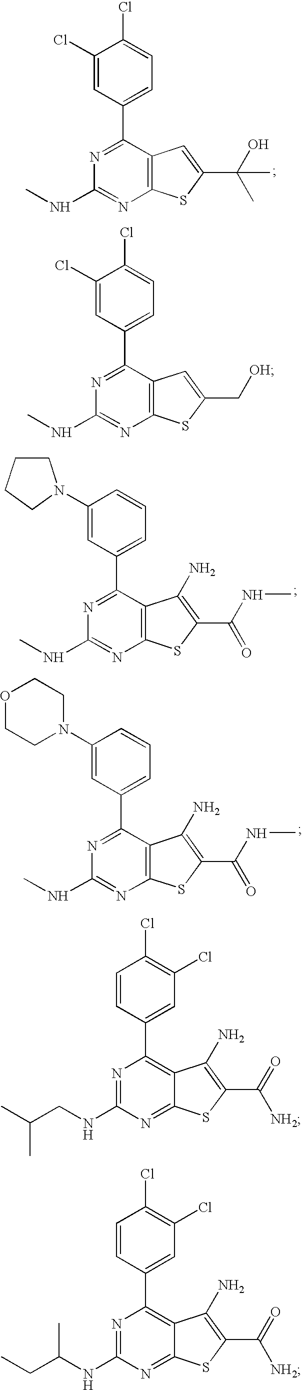

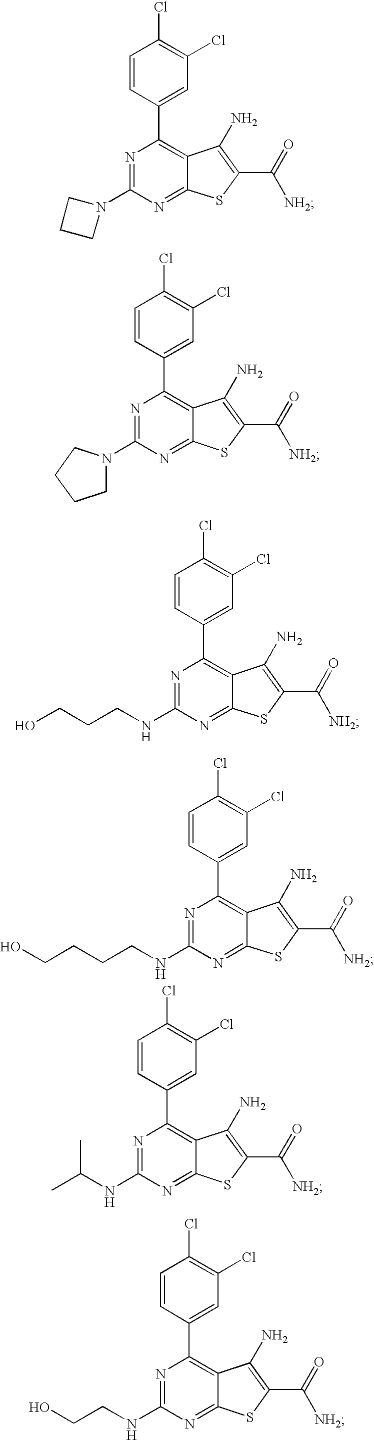

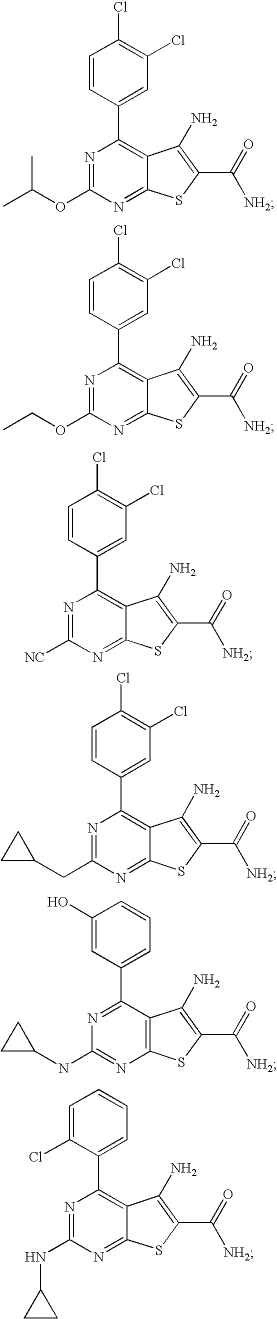

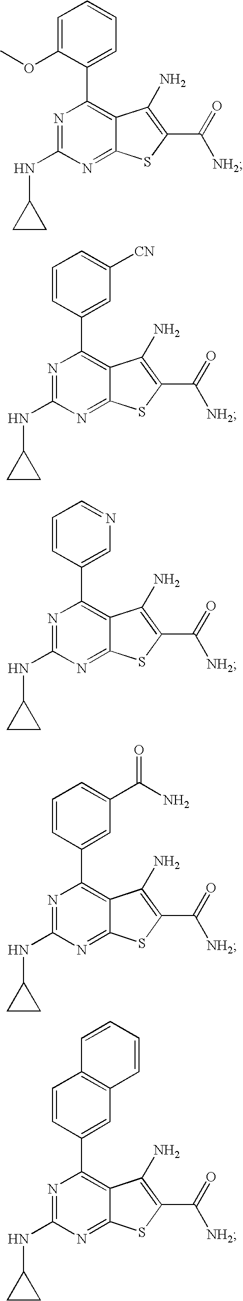

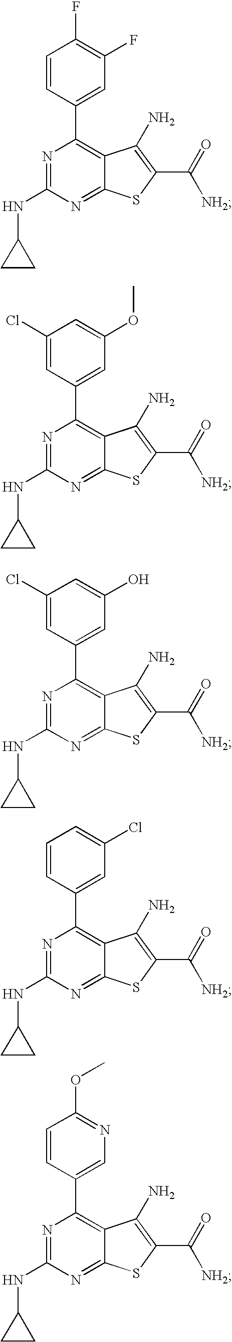

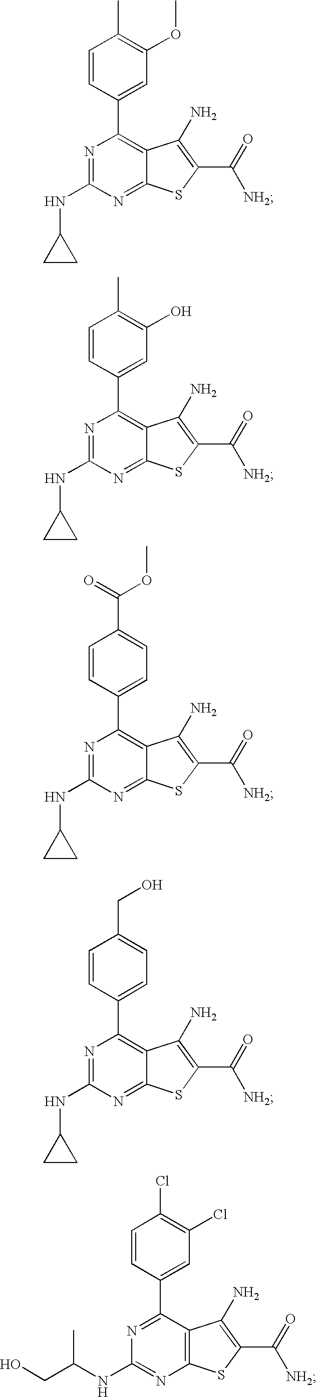

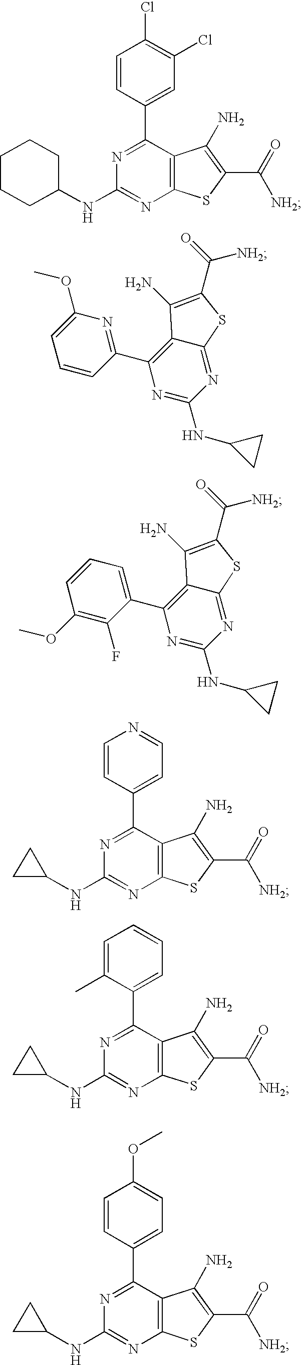

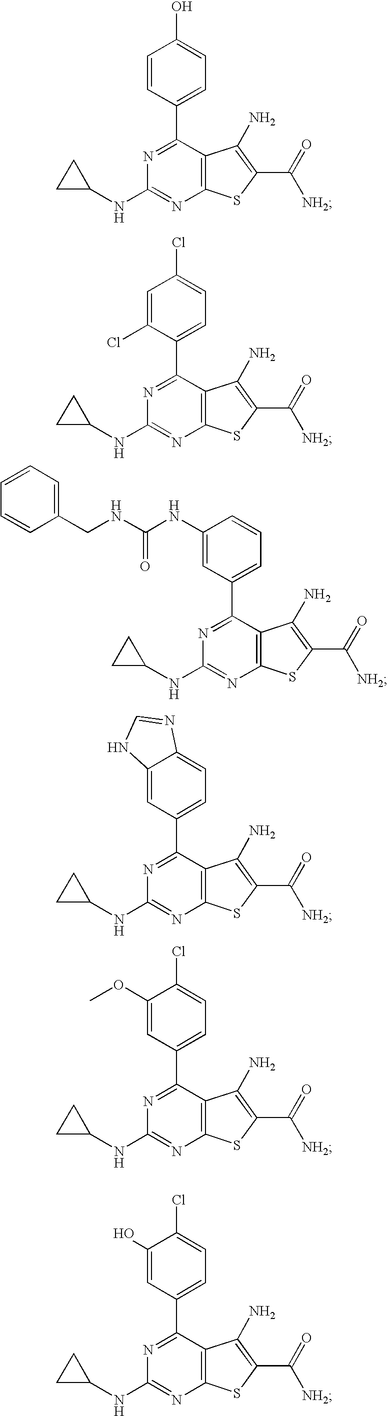

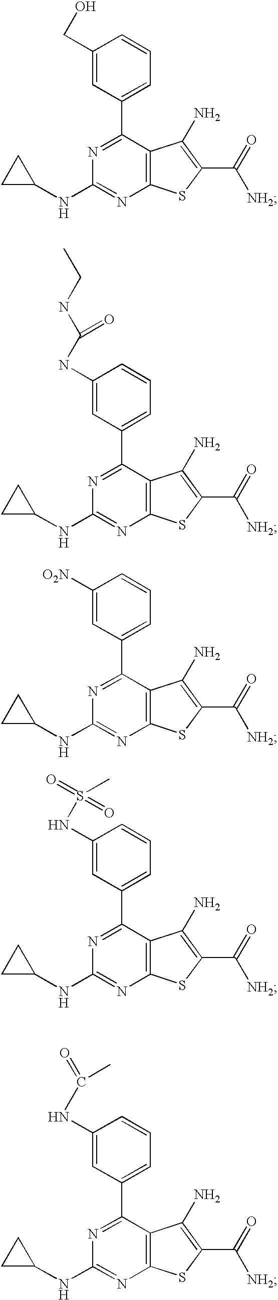

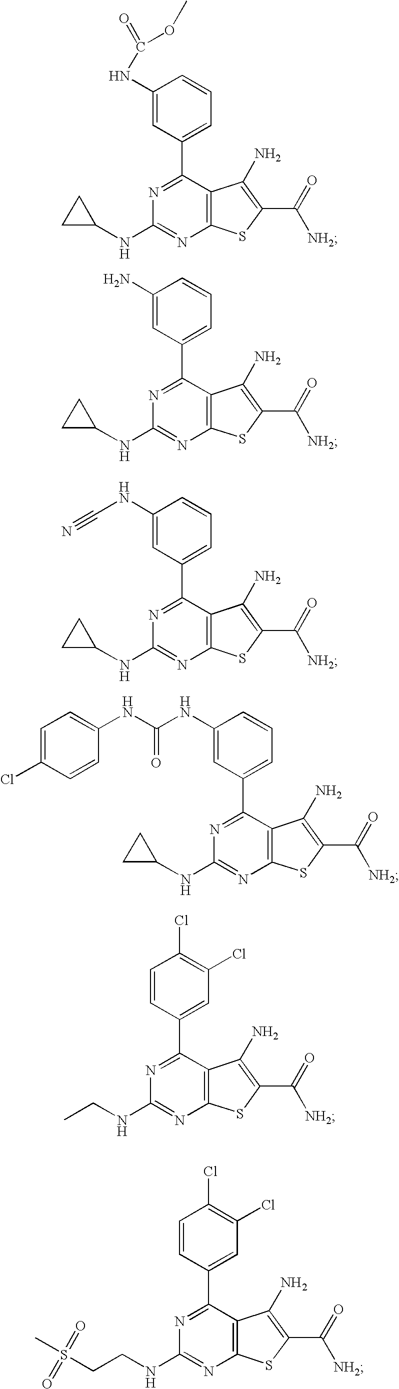

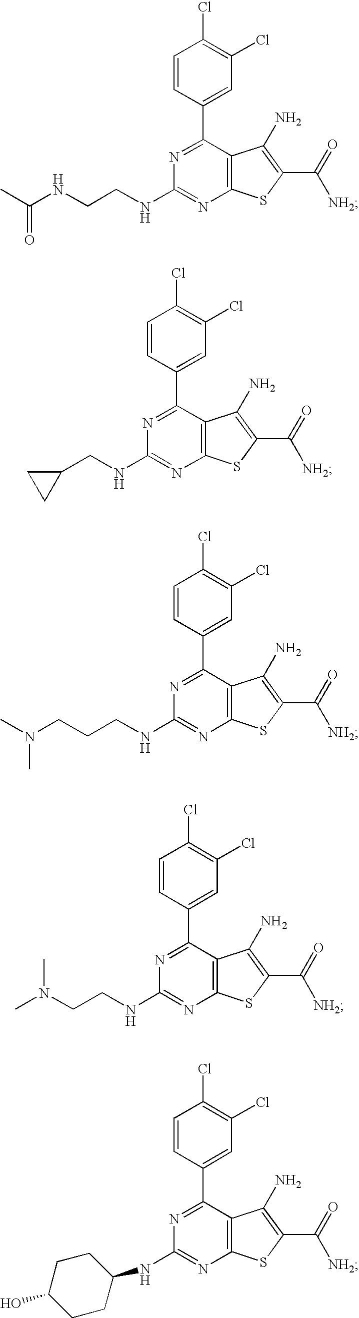

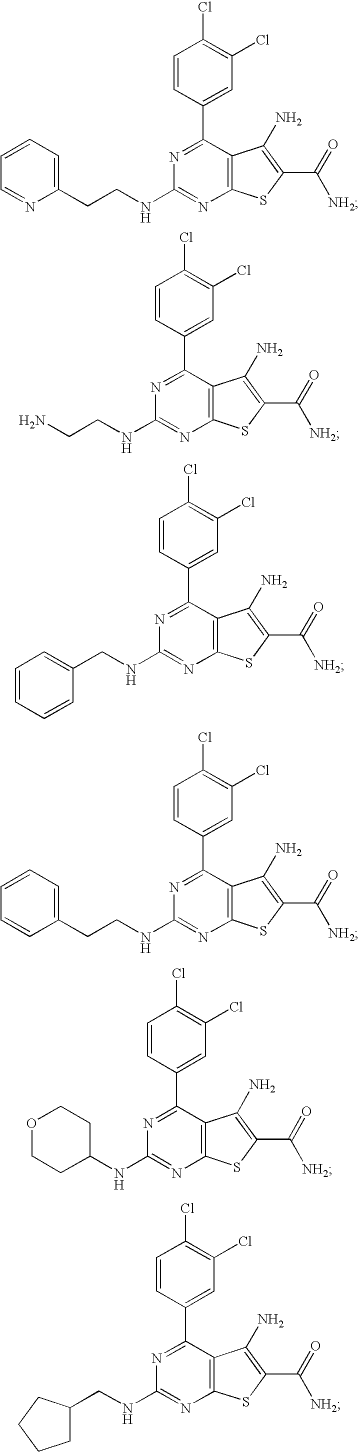

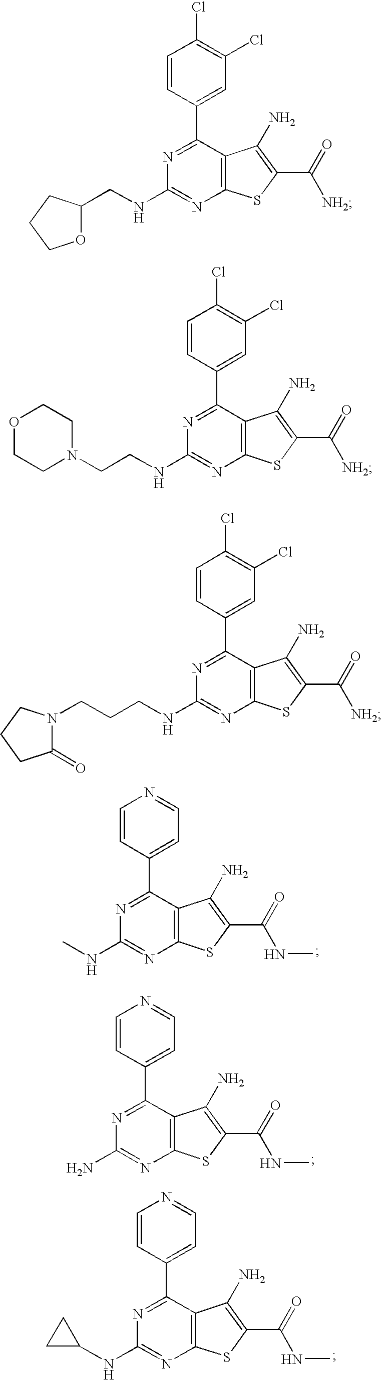

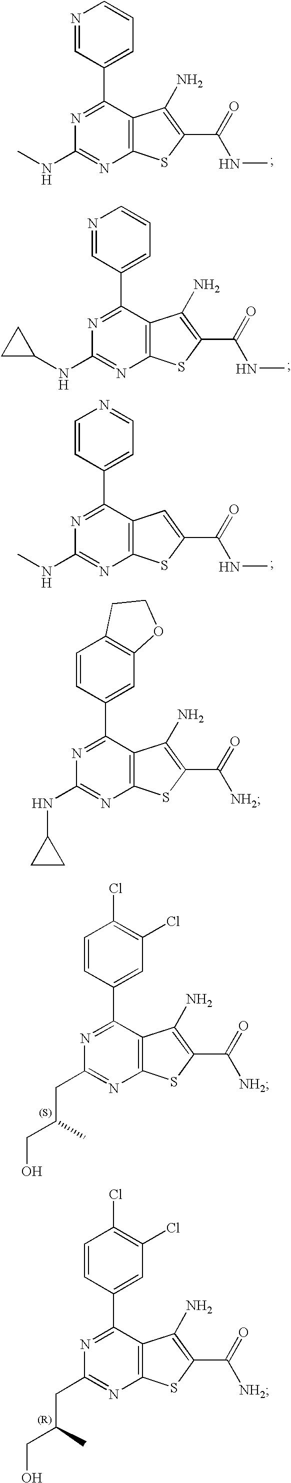

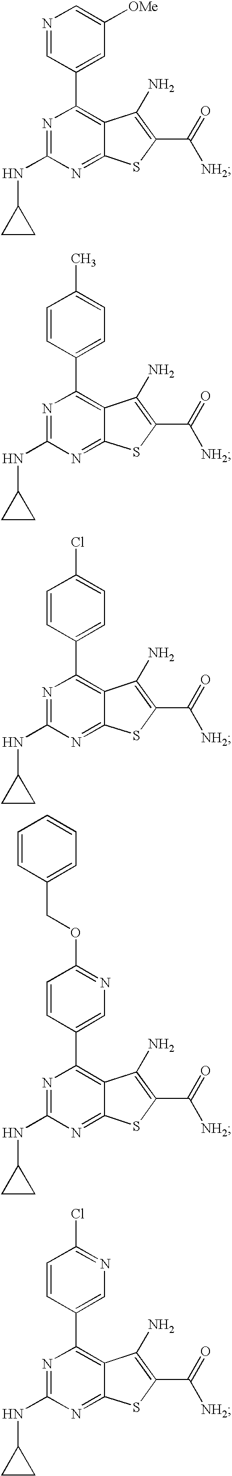

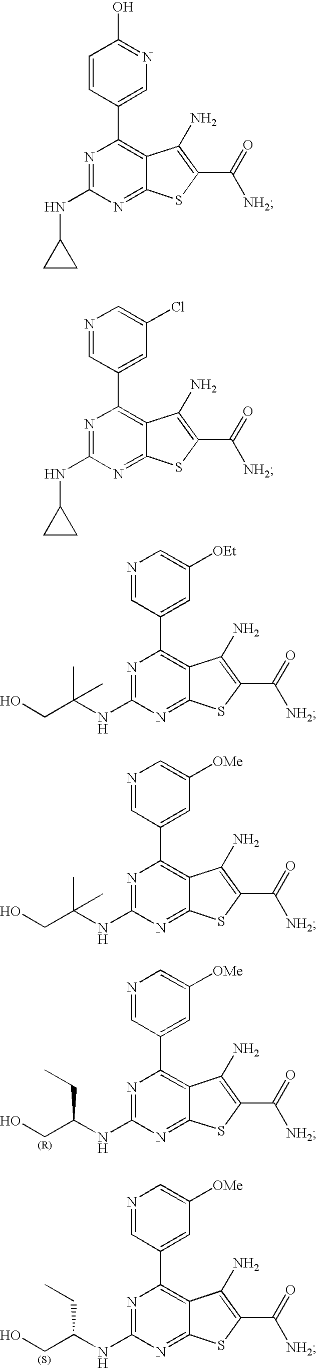

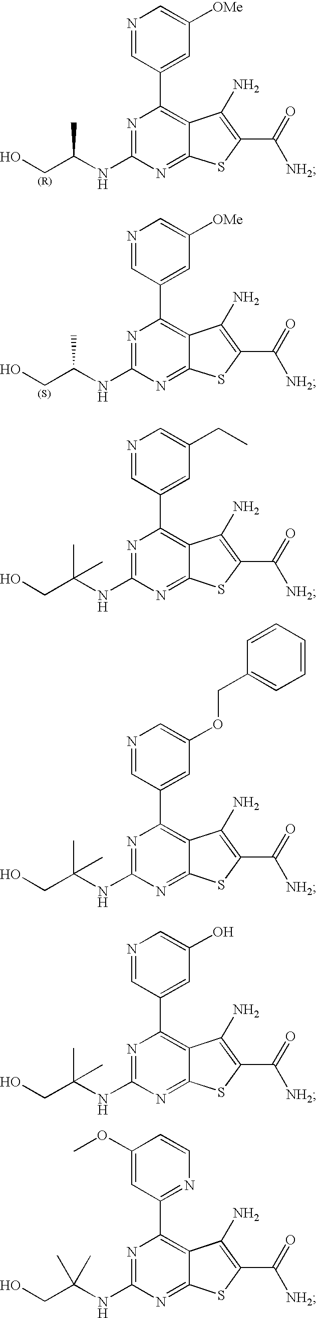

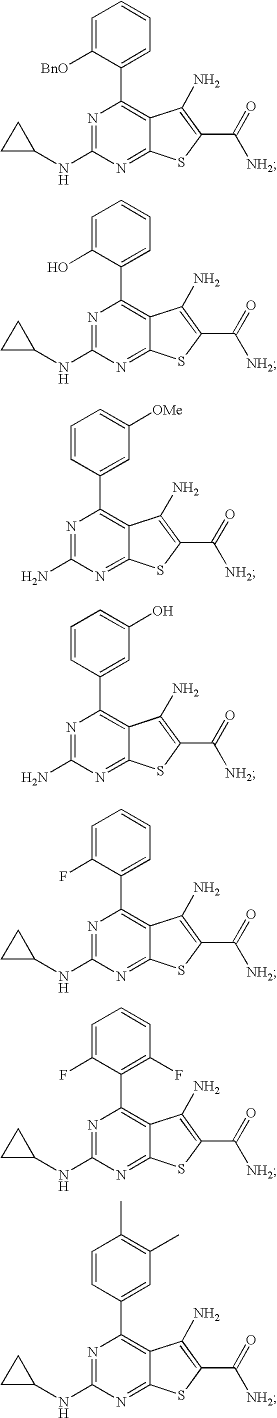

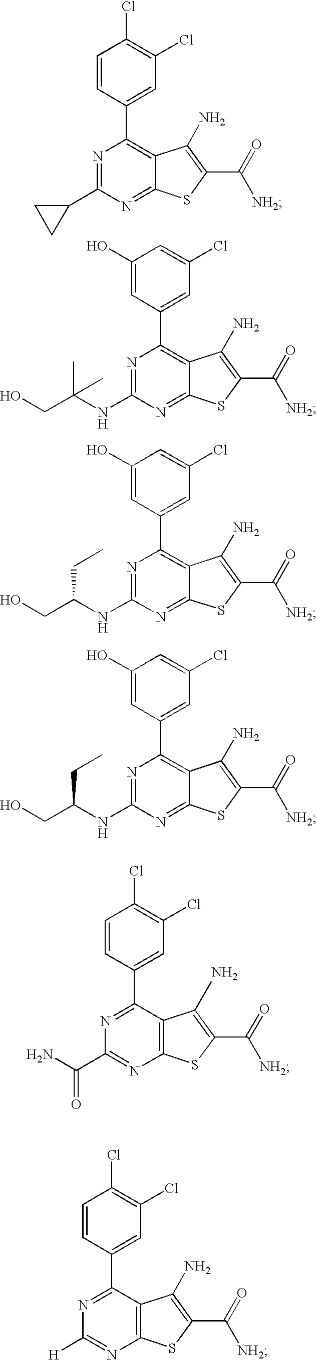

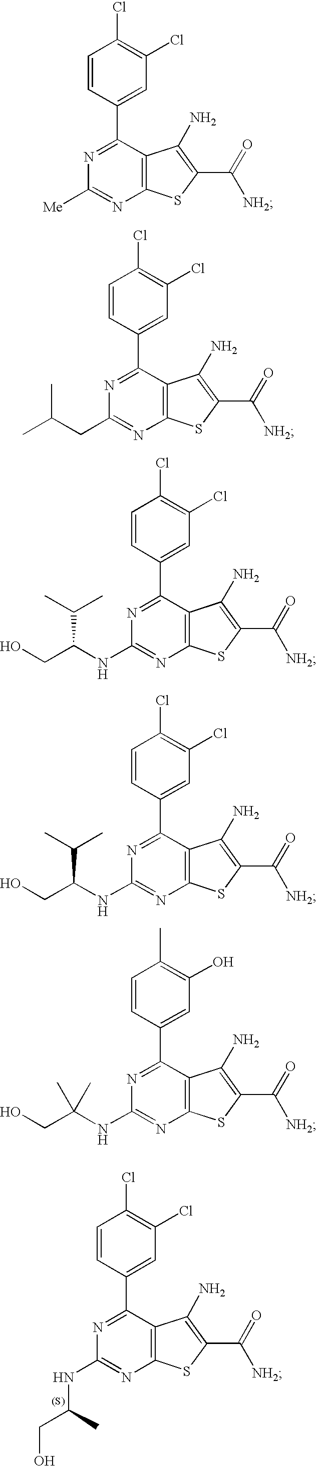

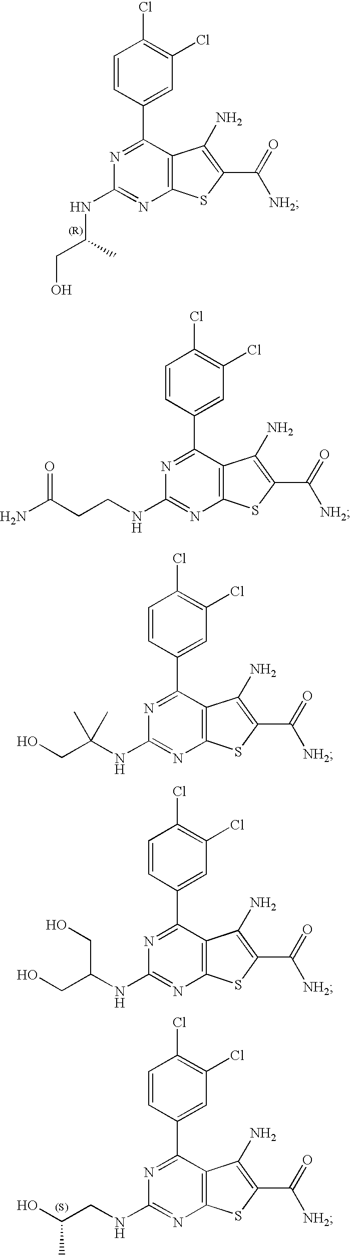

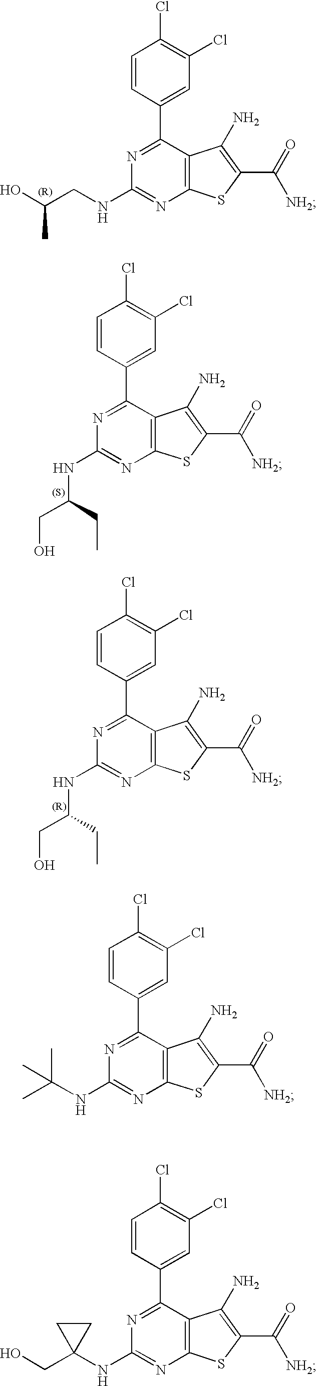

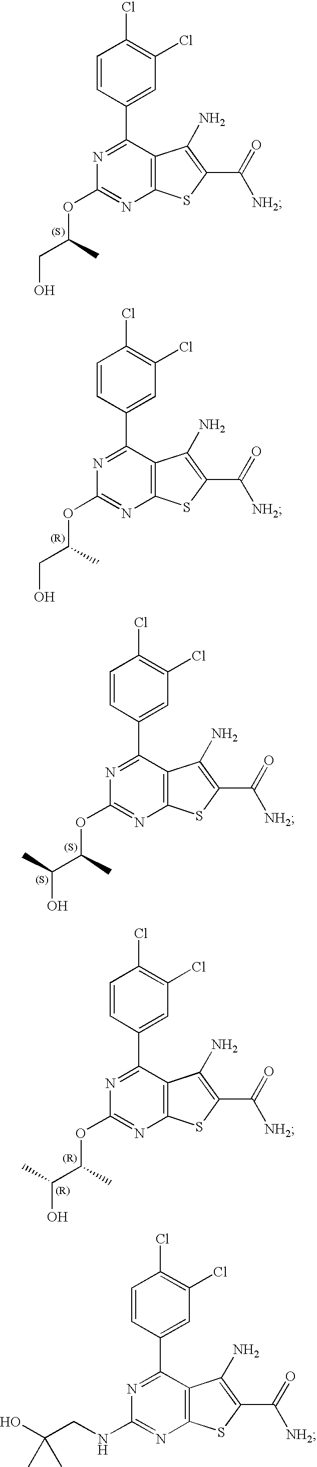

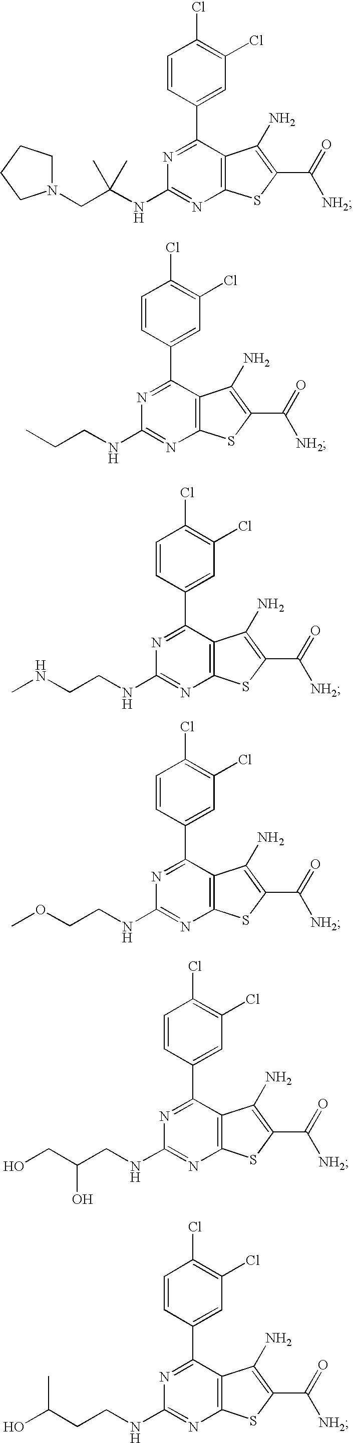

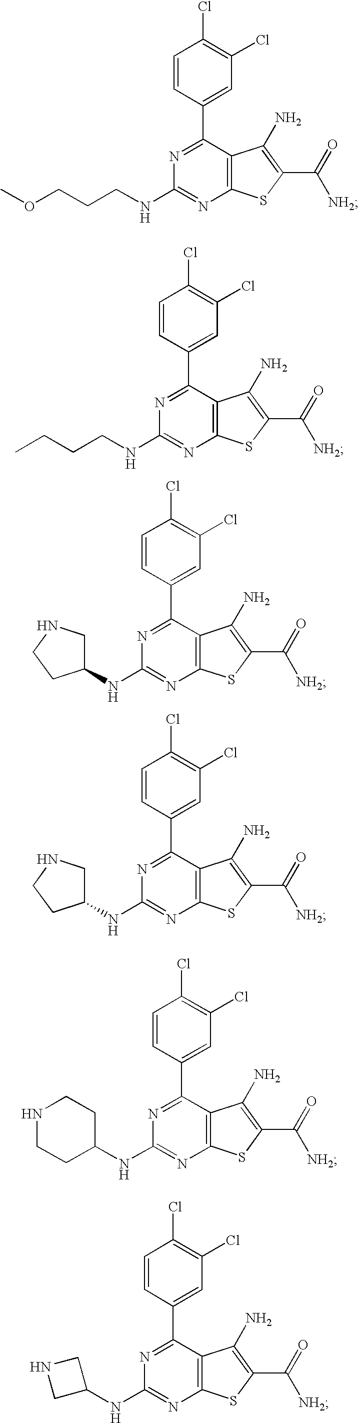

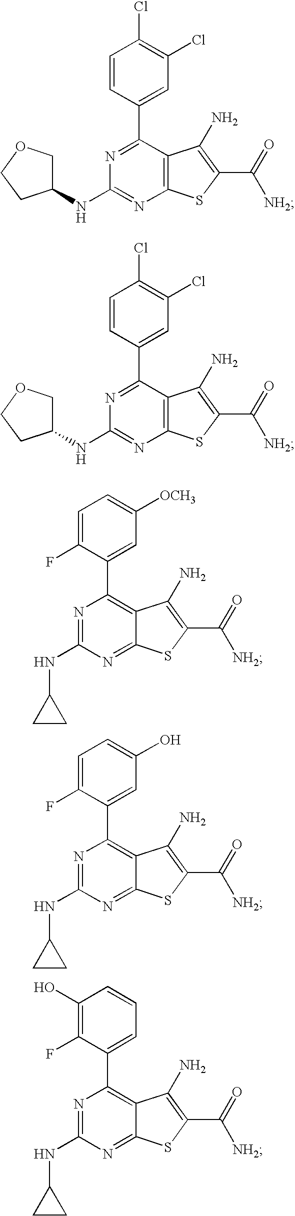

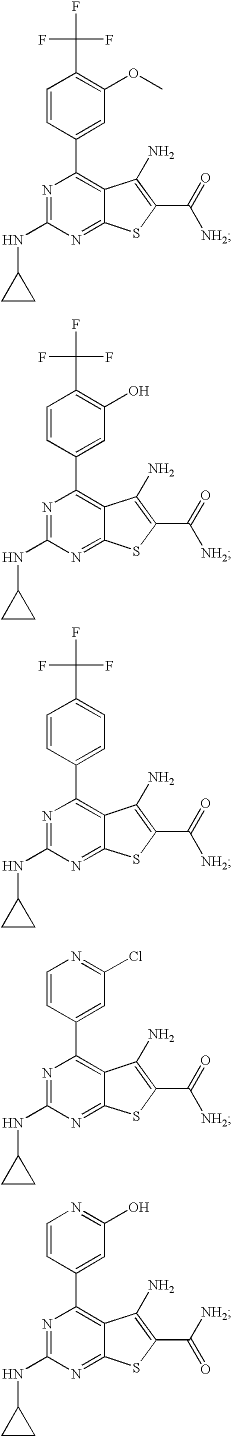

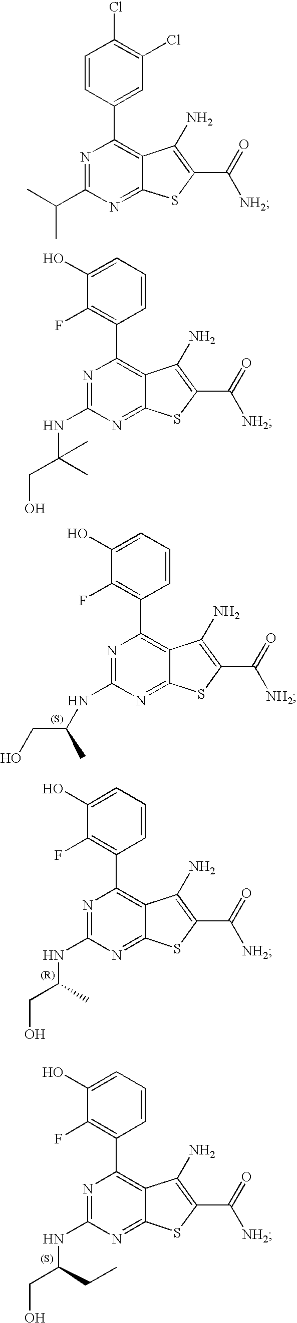

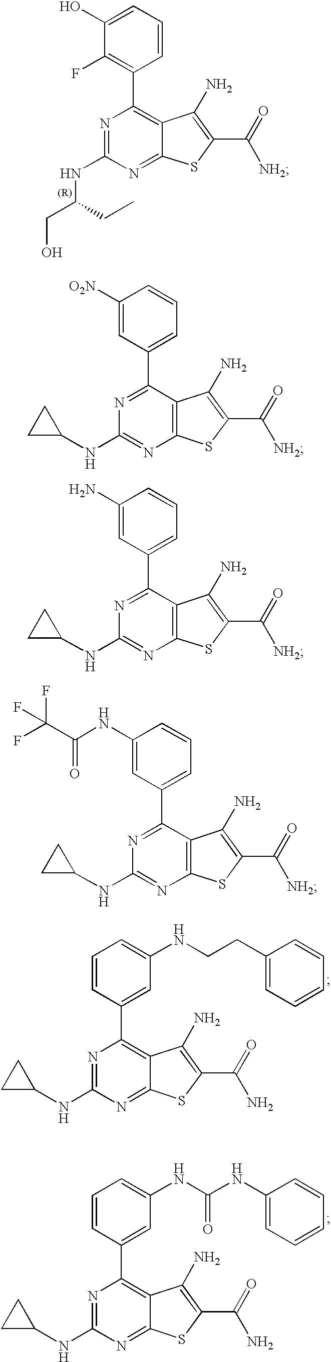

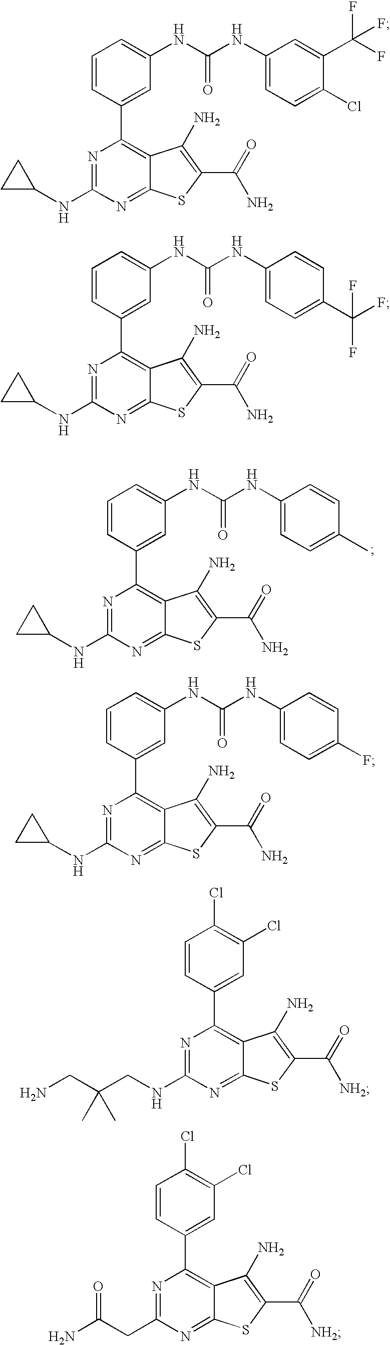

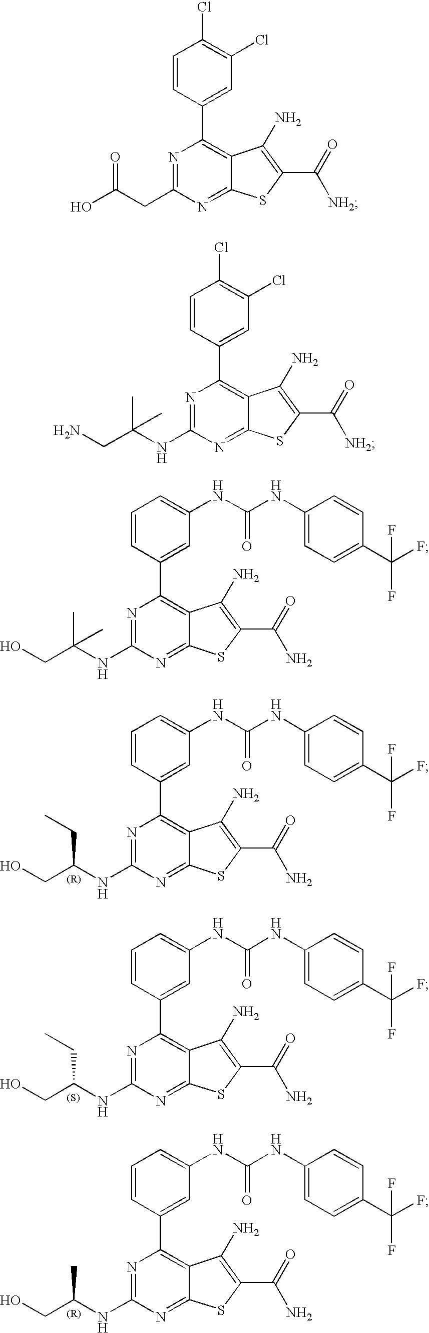

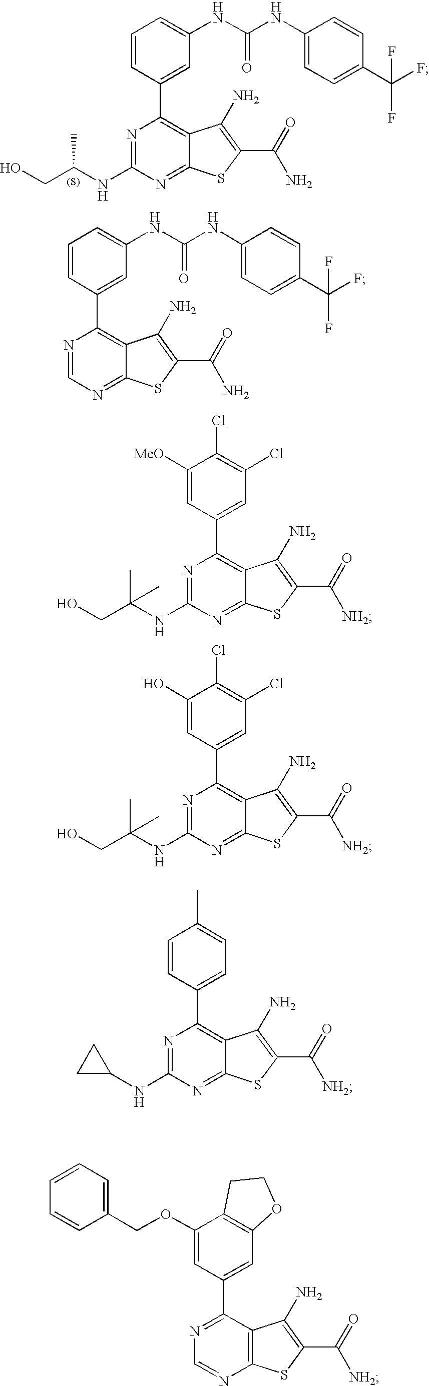

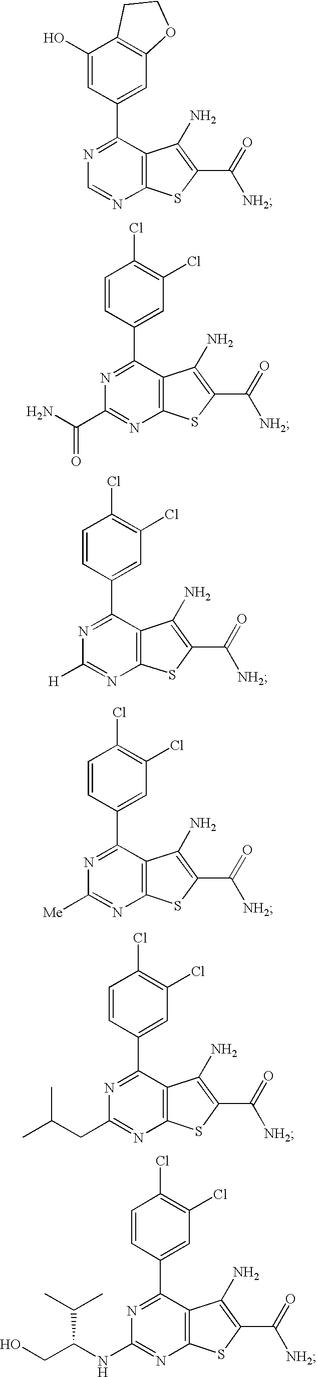

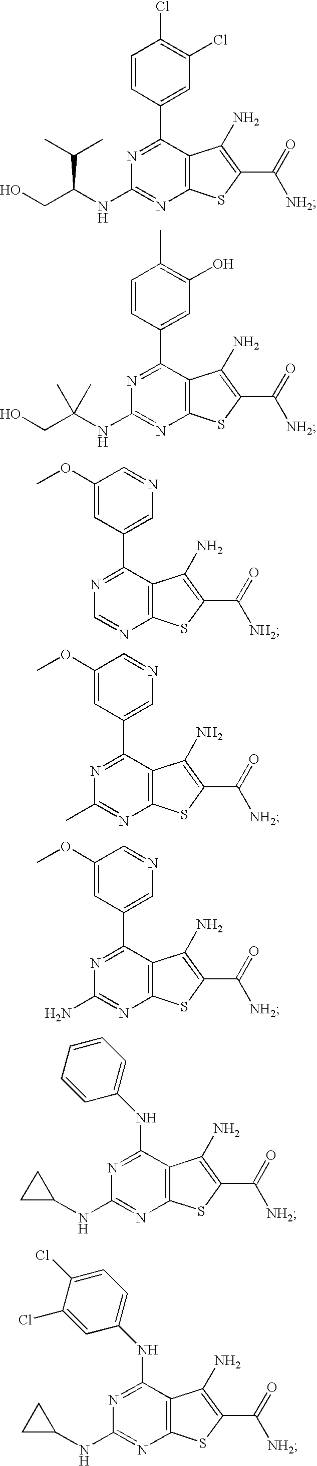

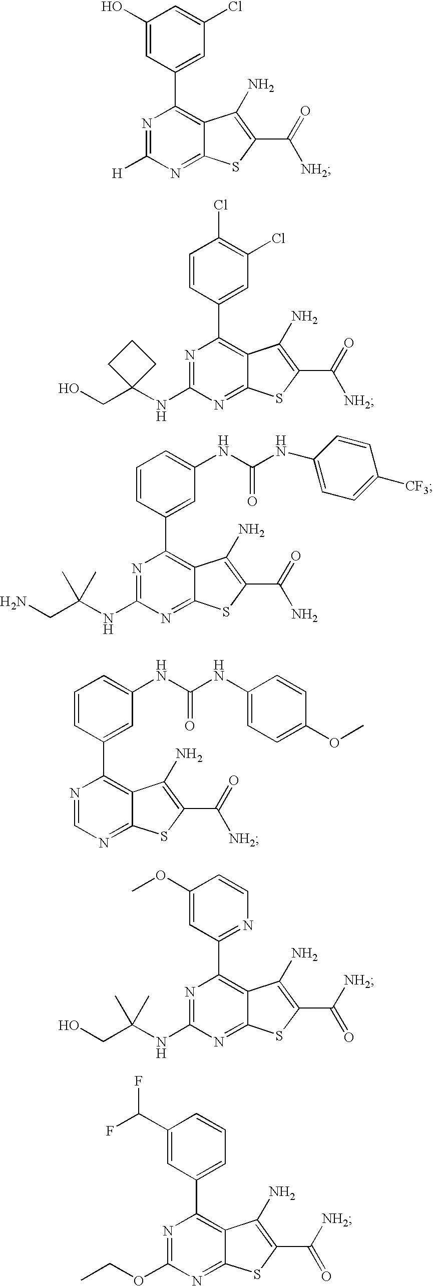

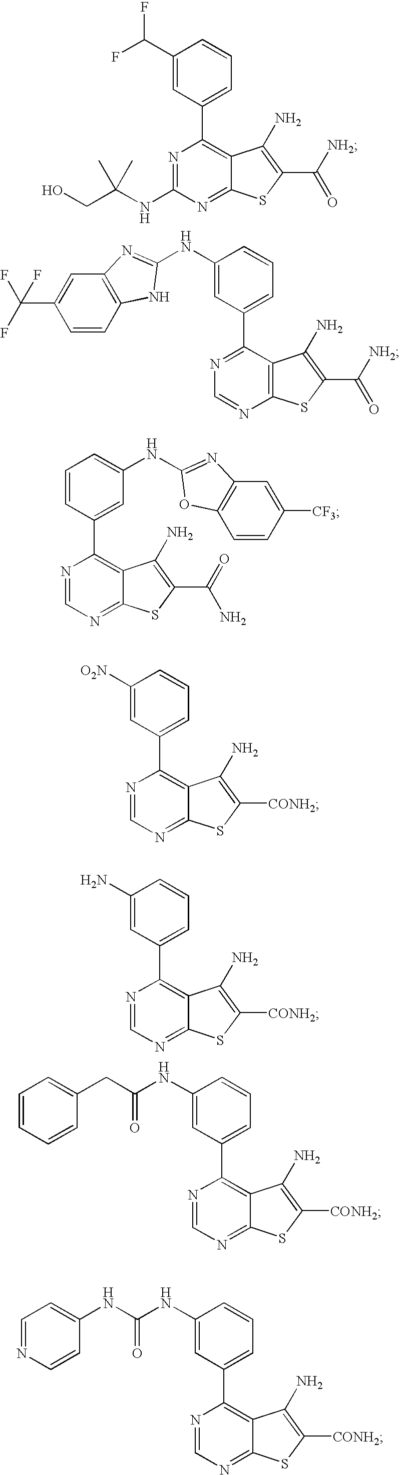

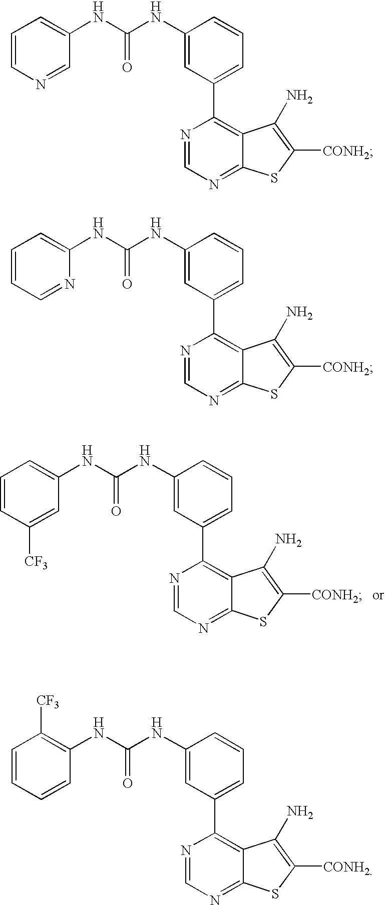

17. A pharmaceutical composition comprising an effective amount of a compound of claim 1, 5, 6, 7, 11, 12, 14, 15 or 16 and a pharmaceutically acceptable carrier.

18. A pharmaceutical composition of claim 17, wherein said composition is suitable for parenteral, transdermal, mucosal, nasal, buccal, rectal, sublingual, or oral administration to a patient.

19. A unit dosage form comprising an effective amount of a compound of claim 1, 5, 6, 7, 11, 12, 14, 15 or 16.

20. A method for treating an inflammatory disorder, a cognition and memory disorder or an autoimmune disorder comprising administering to a patient in need thereof an effective amount of a compound of the formula: 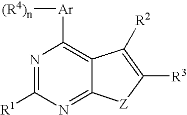
or a pharmaceutically acceptable salt thereof, wherein:
Ar is phenyl, naphthyl, pyridine or dihydrobenzofuran;
R¹ is H, C(O)NR⁵R⁶, NR⁵R⁶, O—C₁₋₈alkyl, CN, or substituted or unsubstituted C₁₋₈alkyl;
R² is H, NH₂, substituted or unsubstituted C₁₋₈alkyl or substituted or unsubstituted aryl;
R³ is C₁₋₈hydroxyalkyl or C(O)NR⁵R⁶;
R⁴ is at each occurrence independently halo, OH, NH₂, CN, NHCN, NO₂, substituted or unsubstituted C₁₋₈alkyl, substituted or unsubstituted O—C₁₋₈alkyl, substituted or unsubstituted O—C₁₋₈alkylene-aryl, substituted or unsubstituted aryl, substituted or unsubstituted O-aryl, substituted or unsubstituted heterocyclyl, NHS(O)₂—C₁₋₈alkyl, NHC(O)—C₁₋₈alkyl, NHC(O)O—C₁₋₈alkyl, NHC(O)NHC₁₋₈alkyl, NHC(O)NHC₀₋₈alkylene-substituted or unsubstituted phenyl, NR⁵R⁶, NHC(O)NHR⁵, NHC(O)R⁵, C(O)OC₁₋₈alkyl, or C(O)NR⁵R⁶; or two adjacent R₄ groups taken together represent —OCH₂O—, —OCH₂CH₂O—, or —NH [symbol] N—; or two adjacent R₄ groups taken together with Ar form naphthalene;
R⁵ and R⁶ are at each occurrence independently H, substituted or unsubstituted C₁₋₈alkyl, substituted or unsubstituted aryl, substituted or unsubstituted heterocyclyl, substituted or unsubstituted C₁₋₈alkylene-O—C₁₋₈alkyl, C₁₋₈alkylene-C(O)NH₂, C₁₋₈alkylene-C(O)OH, substituted or unsubstituted C₃₋₈cycloalkyl; or R⁵ and R⁶ together with the nitrogen atom to which they are attached form a 3-7 membered heterocyclyl;
Z is S, NH or O; and
n is an integer ranging from 0 to 5;
wherein groups that are substituted are substituted with one or more halogen; C₁₋₈ alkyl; C₂₋₈ alkenyl; C₂₋₈ alkynyl; hydroxyl; C₁₋₈ alkoxyl; amino; nitro; thiol; thioether; imine; cyano; amido; phosphonato; phosphine; carboxyl; thiocarbonyl; sulfonyl; sulfonamide; ketone; aldehyde; ester; carbonyl; haloalkyl; B(OH)₂; carbocyclic cycloalkyl, heterocycloalkyl, monocyclic or fused or non-fused polycyclic aryl or heteroaryl; amino; O-lower alkyl; O-aryl, aryl; aryl-lower alkyl; CO₂CH₃; CONH₂; OCH₂CONH₂; NH₂; SO₂NH₂; OCHF₂; CF₃; or OCF₃ groups, wherein each of these groups is optionally substituted.

21. A method for treating, preventing or ameliorating one or more symptoms of cancer comprising administering to a patient in need thereof an effective amount of a compound of claim 1, 5, 6, 7, 11, 12, 14, 15 or 16.

22. A method of claim 21, wherein the cancer is of the head, neck, eye, skin, mouth, throat, esophagus, chest, bone, lung, colon, sigmoid, rectum, stomach, prostate, breast, ovary, testicle, kidney, liver, pancreas, brain, intestine, heart, thyroid or adrenals.

23. A method for inhibiting TNFα in a cell comprising contacting a cell expressing TNFα with an effective amount of a compound of claim 1, 5, 6, 7, 11, 12, 14, 15 or 16.

24. A method for inhibiting PDE4 in a cell comprising contacting a cell expressing PDE4 with an effective amount of a compound of the formula: 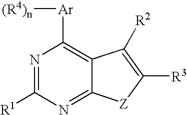
or a pharmaceutically acceptable salt thereof, wherein:
Ar is phenyl, naphthyl, pyridine or dihydrobenzofuran;
R¹ is H, C(O)NR⁵R⁶, NR⁵R⁶, O—C₁₋₈alkyl, CN, or substituted or unsubstituted C₁₋₈alkyl;
R² is H, NH₂, substituted or unsubstituted C₁₋₈alkyl or substituted or unsubstituted aryl;
R³ is C₁₋₈hydroxyalkyl or C(O)NR⁵R⁶;
R⁴ is at each occurrence independently halo, OH, NH₂, CN, NHCN, NO₂, substituted or unsubstituted C₁₋₈alkyl, substituted or unsubstituted O—C₁₋₈alkyl, substituted or unsubstituted O—C₁₋₈alkylene-aryl, substituted or unsubstituted aryl, substituted or unsubstituted O-aryl, substituted or unsubstituted heterocyclyl, NHS(O)₂—C₁₋₈alkyl, NHC(O)—C₁₋₈alkyl, NHC(O)O—C₁₋₈alkyl, NHC(O)NHC₁₋₈alkyl, NHC(O)NHC₀₋₈alkylene-substituted or unsubstituted phenyl, NR⁵R⁶, NHC(O)NHR⁵, NHC(O)R⁵, C(O)OC₁₋₈alkyl, or C(O)NR⁵R⁶; or two adjacent R₄ groups taken together represent —OCH₂O—, —OCH₂CH₂O—, or —NH [symbol] N—; or two adjacent R₄ groups taken together with Ar form naphthalene;
R⁵ and R⁶ are at each occurrence independently H, substituted or unsubstituted C₁₋₈alkyl, substituted or unsubstituted aryl, substituted or unsubstituted heterocyclyl, substituted or unsubstituted C₁₋₈alkylene-O—C₁₋₈alkyl, C₁₋₈alkylene-C(O)NH₂, C₁₋₈alkylene-C(O)OH, substituted or unsubstituted C₃₋₈cycloalkyl; or R⁵ and R⁶ together with the nitrogen atom to which they are attached form a 3-7 membered heterocyclyl;
Z is S, NH or O; and
n is an integer ranging from 0 to 5;
wherein groups that are substituted are substituted with one or more halogen; C₁₋₈ alkyl; C₂₋₈ alkenyl; C₂₋₈ alkynyl; hydroxyl; C₁₋₈ alkoxyl; amino; nitro; thiol; thioether; imine; cyano; amido; phosphonato; phosphine; carboxyl; thiocarbonyl; sulfonyl; sulfonamide; ketone; aldehyde; ester; carbonyl; haloalkyl; B(OH)₂; carbocyclic cycloalkyl, heterocycloalkyl, monocyclic or fused or non-fused polycyclic aryl or heteroaryl; amino; O-lower alkyl; O-aryl, aryl; aryl-lower alkyl; CO₂CH₃; CONH₂; OCH₂CONH₂; NH₂; SO₂NH₂; OCHF₂; CF₃; or OCF₃ groups, wherein each of these groups is optionally substituted.

25. A method for inhibiting B-RAF in a cell comprising contacting a cell expressing B-RAF with an effective amount of a compound of the formula: 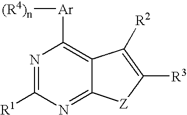
or a pharmaceutically acceptable salt thereof, wherein:
Ar is phenyl, naphthyl, pyridine or dihydrobenzofuran;
R¹ is H, C(O)NR⁵R⁶, NR⁵R⁶, O—C₁₋₈alkyl, CN, or substituted or unsubstituted C₁₋₈alkyl;
R² is H, NH₂, substituted or unsubstituted C₁₋₈alkyl or substituted or unsubstituted aryl;
R³ is C₁₋₈hydroxyalkyl or C(O)NR⁵R⁶;
R⁴ is at each occurrence independently halo, OH, NH₂, CN, NHCN, NO₂, substituted or unsubstituted C₁₋₈alkyl, substituted or unsubstituted O—C₁₋₈alkyl, substituted or unsubstituted O—C₁₋₈alkylene-aryl, substituted or unsubstituted aryl, substituted or unsubstituted O-aryl, substituted or unsubstituted heterocyclyl, NHS(O)₂—C₁₋₈alkyl, NHC(O)—C₁₋₈alkyl, NHC(O)O—C₁₋₈alkyl, NHC(O)NHC₁₋₈alkyl, NHC(O)NHC₀₋₈alkylene-substituted or unsubstituted phenyl, NR⁵R⁶, NHC(O)NHR⁵, NHC(O)R⁵, C(O)OC₁₋₈alkyl, or C(O)NR⁵R⁶; or two adjacent R₄ groups taken together represent —OCH₂O—, —OCH₂CH₂O—, or —NH [symbol] N—; or two adjacent R₄ groups taken together with Ar form naphthalene;
R⁵ and R¹ are at each occurrence independently H, substituted or unsubstituted C₁₋₈alkyl, substituted or unsubstituted aryl, substituted or unsubstituted heterocyclyl, substituted or unsubstituted C₁₋₈alkylene-O—C₁₋₈alkyl, C₁₋₈alkylene-C(O)NH₂, C₁₋₈alkylene-C(O)OH, substituted or unsubstituted C₃₋₈cycloalkyl; or R⁵ and R⁶ together with the nitrogen atom to which they are attached form a 3-7 membered heterocyclyl;
Z is S, NH or O; and
n is an integer ranging from 0 to 5;
wherein groups that are substituted are substituted with one or more halogen; C₁₋₈ alkyl; C₂₋₈ alkenyl; C₂₋₈ alkynyl; hydroxyl; C₁₋₈ alkoxyl; amino; nitro; thiol; thioether; imine; cyano; amido; phosphonato; phosphine; carboxyl; thiocarbonyl; sulfonyl; sulfonamide; ketone; aldehyde; ester; carbonyl; haloalkyl; B(OH)₂; carbocyclic cycloalkyl, heterocycloalkyl, monocyclic or fused or non-fused polycyclic aryl or heteroaryl; amino; O-lower alkyl; O-aryl, aryl; aryl-lower alkyl; CO₂CH₃; CONH₂; OCH₂CONH₂; NH₂; SO₂NH₂; OCHF₂; CF₃; or OCF₃ groups, wherein each of these groups is optionally substituted.
